SEQUENCE LISTING (589 sequences)

SEQ ID NO: 1 - DNA, 1606 bp, Homo sapiens
gaggaaagaaagggaaggcaaccgggcagcccaggccccgccccgccgctcccccacccgtgcgcttataaagcacaggaaccagagctggccactcagtggtttcttggtgacactggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctcccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaaccaataaactgtctcaatgctgattcataaaaaaaaaaaaaaaa

SEQ ID NO: 2 - DNA, 2238 bp, Homo sapiens
aaaggacttgtttcctgctgaaaaagcagaaagagattaccagccacagacgggtcatgagcgcggtattactgctggccctcctggggttcatcctcccactgccaggagtgcaggcgctgctctgccagtttgggacagttcagcatgtgtggaaggtgtccgacctgccccggcaatggacccctaagaacaccagctgcgacagcggcttggggtgccaggacacgttgatgctcattgagagcggaccccaagtgagcctggtgctctccaagggctgcacggaggccaaggaccaggagccccgcgtcactgagcaccggatgggccccggcctctccctgatctcctacaccttcgtgtgccgccaggaggacttctgcaacaacctcgttaactccctcccgctttgggccccacagcccccagcagacccaggatccttgaggtgcccagtctgcttgtctatggaaggctgtctggaggggacaacagaagagatctgccccaaggggaccacacactgttatgatggcctcctcaggctcaggggaggaggcatcttctccaatctgagagtccagggatgcatgccccagccagtttgcaacctgctcaatgggacacaggaaattgggcccgtgggtatgactgagaactgcgatatgaaagattttctgacctgtcatcgggggaccaccattatgacacacggaaacttggctcaagaacccactgattggaccacatcgaataccgagatgtgcgaggtggggcaggtgtgtcaggagacgctgctgctcctagatgtaggactcacatcaaccctggtggggacaaaaggctgcagcactgttggggctcaaaattcccagaagaccaccatccactcagcccctcctggggtgcttgtggcctcctatacccacttctgctcctcggacctgtgcaatagtgccagcagcagcagcgttctgctgaactccctccctcctcaagctgcccctgtcccaggagaccggcagtgtcctacctgtgtgcagccccttggaacctgttcaagtggctccccccgaatgacctgccccaggggcgccactcattgttatgatgggtacattcatctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaactctattacccccacgattcttcaccgctgctgaccacccacactcaacctccctctgacctcataacctaatggccttggacaccagattctttcccattctgtccatgaatcatcttccccacacacaatcattcatatctactcacctaacagcaacactggggagagcctggagcatccggacttgccctatgggagaggggacgctggaggagtggctgcatgtatctgataatacagaccctgtcctttctcccagtgctgggatttctccatgtgagggggcagcaggacacccagggatctagcgtgggggaggagaggagcctaatgagaaaatgaccatctaaagcctgcccttcattggtctggttcacgtctccaaaccagcttggatggtagcagagacttcagggtgctccagccaaacgtatttgggcatcaccatgacctgggaggggaagatgcactgagacgtatgaggcttccagcctagcagccagggccctagcacaaacaggaggctcgccccatctgagcaactgcaggagaggttagtacagtcatgcattgcttaacgacagggacgtgtcgttagaaatgtgtcgttaggtgattttatgaccataggaacattgtagcgtgcacttacaccaacccagatggtacagcccaatacacacccaggatggacgctagagtcgactgctcctaggctacaagcctgcagtgcatgttatggtgtgaatactgcaggcaatcttaacaccacggcaagtatttgtgcatctacacacatctaaacatagaaaaggtacagcataaatacactattgtcatctcagcagaaaaaaaaaaaaaaaaa

SEQ ID NO: 3 - DNA, 4285 bp, Homo sapiens
aaaggcgcaagaagcgggcaccccgggaaccccattccctcggctcactcggcgcggagaagcgacgcccgctgactccgagagccccggtgctccgtgcacctggtccccaagttgaggagcgacacccctccacaggggactagcccgcgcggggagcattccggtctcactgaccccggcccacccgcgggactccaggcacctcttctgcccgcaccccgcgacccctcccgggaccccggagacagccggcctgcccccggcgtcccccttggccagcacgccatgccggggctgcgccgggaccgcctactgactctgctgctgctgggcgcgctgctctccgccgacctctacttccacctctggccccaagtacagcgccagctgcggcctcgggagcgcccgcgggggtgcccgtgcaccggccgcgcctcctccctggcgcgggactcggccgcagctgcctcggaccccggcacgatcgtgcacaacttttcccgaaccgagccccggactgaaccggctggcggcagccacagcgggtcgagctccaagttgcaggccctcttcgcccacccgctgtacaacgtcccggaggagccgcctctcctgggagccgaggactcgctcctggccagccaggaggcgctgcggtattaccggaggaaggtggcccgctggaacaggcgacacaagatgtacagagagcagatgaaccttacctccctggaccccccactgcagctccgactcgaggccagctgggtccagttccacctgggtattaaccgccatgggctctactcccggtccagccctgttgtcagcaaacttctgcaagacatgaggcactttcccaccatcagtgctgattacagtcaagatgagaaagccttgctgggggcatgtgactgcacccagattgtgaaacccagtggggtccacctcaagctggtgctgaggttctcggatttcgggaaggccatgttcaaacccatgagacagcagcgagatgaggagacaccagtggacttcttctacttcattgactttcagagacacaatgctgagatcgcagctttccatctggacaggattctggacttccgacgggtgccgccaacagtggggaggatagtaaatgtcaccaaggaaatcctagaggtcaccaagaatgaaatcctgcagagtgttttctttgtctctccagcgagcaacgtgtgcttcttcgccaagtgtccatacatgtgcaagacggagtatgctgtctgtggcaacccacacctgctggagggttccctctctgccttcctgccgtccctcaacctggcccccaggctgtctgtgcccaacccctggatccgctcctacacactggcaggaaaagaggagtgggaggtcaatcccctttactgtgacacagtgaaacagatctacccgtacaacaacagccagcggctcctcaatgtcatcgacatggccatcttcgacttcttgatagggaatatggaccggcaccattatgagatgttcaccaagttcggggatgatgggttccttattcaccttgacaacgccagagggttcggacgacactcccatgatgaaatctccatcctctcgcctctctcccagtgctgcatgataaaaaagaaaacacttttgcacctgcagctgctggcccaagctgactacagactcagcgatgtgatgcgagaatcactgctggaagaccagctcagccctgtcctcactgaaccccacctccttgccctggatcgaaggctccaaaccatcctaaggacagtggaggggtgcatagtggcccatggacagcagagtgtcatagtcgacggcccagtggaacagttggccccagactctggccaggctaacttgacaagctaagggctggcagagtccagtttcagaaaatacgcctggagccagagcagtcgactcgagtgccgaccctgcgtcctcactcccacctgttactgctgggagtcaagtcagctaggaaggaagcaggacattttctcaaacagcaagtggggcccatggaactgaatctttactccttggtgcaccgcttctgtcgtgcgttgccttgctccgtttttcccaaaaagcactggcttcatcaaggccaccgacgatctcctgagtgcactgggaaatctgggtataggtcaggcttggcagccttgatcccaggagagtactaatggtaacaagtcaaataaaaggacatcaagtggatacctgacttctcaggatccttattctagctacaagtcaaagataactcctggtccagacaaaacacctggcctatcacaagctgactaaaaatctgcactttgggccagcgcaggcaacagtaactctgacaggttcaaattagacctcacactttctactcatattctagtcactggacccatctgaatcagtaatccctactgcccggtcctggagtaacttcttagagatattataacaagtggcaaaaataaaagagggatttgctaagaatatcagaaaaggagtgttccaatttgaagagtattacaattgaaataacatcaaatatgtcacactaagcagccagtaacagaataaataattacaacgaaggaaaaaaaaaggaaagtcctccaaggtcaggatggcatgggaacaggcctagcagggacacaagcctggagtaaggcaggaaaagagccaaggctgactacagcccaccaaccacaatcttcttccctaagaccccaggattgtccccggcccatccccaaaagaagaaaggggctatgtggaaaggtgaggccctctagatgcccctccctgtgacggctggctcaaaaaagactaagtcaggttttttattgaagcctcccttagaagaaagtgtagtagggattccttgtctcctctaccttctccctgacttgttgatatctgaaactccttttaataggaggctttgtttcttattccataattaatgaccgaaaacacaaaggacagtaaggtcttcattccatggagtgactagtcagtcaacaaaagtaaaaatgcctcatttcatcaaatcaacttttccagtaaaggccagagttcaaatactgtaagcatgagaatagaatggagctgtcactaaagagagtacaaggtcaagaaccagaaatgtctccaagctttctgcagtgtgagcaaagatagcctcgactggacaaataacacttctgaccccgtggtacccccacatgactggtatgctgcccctggactttgtgtctctctaaaatcactaccattcactgagatctgaccaggtgaggcactcaccaacgctacagaagcaacacacaggtttccaatcctctcaaacacagatacaacccccatcctccaatgtgcatggaagaactttaaatctcagagagagtaagcaaaatcacacaatttagtggtttagacctattccaaagtcttcttttcactgtaacacaaaacccaacagtatcccacccaatttatgaagtcaaatttgtttgcttaaaaaaaaaatcagctgggcatgggctcacacctgtaatcccagcactttgggcctaaacaggcagatcgcttggtctcaggagctcgagaccagcctgggtaacatgacaaaaccctgtctctacaaaaaaatacaaaaaaaaaaaaatttttttttaaatcaactctttaacagagaaaccttaatcccaaagatttccttaactgttgtggccctactgcagcatagggctccccacttcattcactgtctctcctgcttcaagatgtttcttccctgtggatgaagaaccaggagatcacacacagaaatgggaaaatatctgggtaaaaacattcaaagaatagcacaaaatggtttgggacttccaaataagactctcaggaaagcaggctacagctctacctcaaaacccgccacctcctataacgaaggactggttttccaatggcctatatacacattttgctgcactctacaaacagaaagcaataggcttgaatttcaccatgatatgtggtacacctaccttattattcacttatctttcaaccttccattactttttgattagtaacaaaattaagactcatttctcaaaaatatcagaaaaccaaccgtttcattcttactcgagttcttccagcatgttcattttgaactgtttacctcccatgaaataaaaagtctcttgactctgaaaaaaaaaaa

SEQ ID NO: 4 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 5 - DNA, 1531 bp, Homo sapiens
aaaagtcattgaagagcttgtggggctgtgggacctgcgccctctggaggaattccatacacccactcaactctggcaaataggaaattgtcaagtaggagacaaggagcaaagtcctatcacagcgggaggggacgccagcgcctgcagaggctgagcagggaaaaagccagtgccccagcggaagcacagctcagagctggtctgccatggacatcctggtcccactcctgcagctgctggtgctgcttcttaccctgcccctgcacctcatggctctgctgggctgctggcagcccctgtgcaaaagctacttcccctacctgatggccgtgctgactcccaagagcaaccgcaagatggagagcaagaaacgggagctcttcagccagataaaggggcttacaggagcctccgggaaagtggccctactggagctgggctgcggaaccggagccaactttcagttctacccaccgggctgcagggtcacctgcctagacccaaatccccactttgagaagttcctgacaaagagcatggctgagaacaggcacctccaatatgagcggtttgtggtggctcctggagaggacatgagacagctggctgatggctccatggatgtggtggtctgcactctggtgctgtgctctgtgcagagcccaaggaaggtcctgcaggaggtccggagagtactgagaccgggaggtgtgctctttttctgggagcatgtggcagaaccatatggaagctgggccttcatgtggcagcaagttttcgagcccacctggaaacacattggggatggctgctgcctcaccagagagacctggaaggatcttgagaacgcccagttctccgaaatccaaatggaacgacagccccctcccttgaagtggctacctgttgggccccacatcatgggaaaggctgtcaaataatctttcccaagctccaaggcactcatttgctccttccccagcctccaattagaacaagccacccaccagcctatctatcttccactgagagggacctagcagaatgagagaagacattcatgtaccacctactagtccctctctccccaacctctgccagggcaatctctaacttcaatcccgccttcgacagtgaaaaagctctacttctacgctgacccagggaggaaacactaggaccctgttgtatcctcaactgcaagtttctggactagtctcccaacgtttgcctcccaatgttgtccctttccttcgttcccatggtaaagctcctctcgctttcctcctgaggctacacccatgcgtctctaggaactggtcacaaaagtcatggtgcctgcatccctgccaagcccccctgaccctctctccccactaccaccttcttcctgagctgggggcaccagggagaatcagagatgctggggatgccagagcaagactcaaagaggcagaggttttgttctcaaatattttttaataaatagacgaaaccacgaaaaaaaaaaaaaaaaaaaaaaaaaaaa

SEQ ID NO: 6 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 7 - DNA, 478 bp, Homo sapiens
gtgtgccggatttggttagctgagcccaccgagaggcgcctgcaggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatg

SEQ ID NO: 8 - DNA, 4782 bp, Homo sapiens
ttgggggatgccgacgcctgggccttgaggatgctgcggaagctcaccatcgagcagatcaacgactggttcaccatcggcaagacggtgaccaatgtggagctgctgggcgcgccgcccgccttcccggcaggggcggccagggaggaggtgcagcgccaggacgtggcccccggcgctggtcccgcggcccaggctccggctctggcccaagctccggcccggccggccgctgcgttcgaaaggtcactcagccaaaggagctcagaagtagagtatattaacaaatacagacagctcgaagcacaagagcttgatgtgtgtcgcagtgtccaaccaaccagtgggccaggtccaaggccatctttggctaaattaagcagtgtgacgtgcatcccagggacaacttataaatatcctgatttgcctataagtcgatacaaggaagaggttgtgtctttgatagaaagtaattccgtggtgattatccatggggccacgggaagcggtaaaagcactcagctcccgcagtatatcttggaccactacgttcagcgctccgcctactgcagcattgtggtcacccagccccggaagataggggcaagcagcatcgccaggtggatcagtaaagagcgtgcctggaccctgggaggtgtggtgggctaccaggtagggctagagaaaatagcaacagaggacaccaggctaatttatatgacaactggagtcctgcttcagaaaatagttagtgccaagagtttgatggaattcacacatatcatcattgatgaagtacacgaacgaacagaagaaatggatttcctgctattggtagtccgcaaactcttaagaacaaattcacgttttgtgaaggtggtcctgatgtcggctaccatcagctgtaaagagtttgcagactactttgctgttcctgttcaaaacaagatgaatcctgcatatatttttgaagtggaaggcaagccccattcagttgaagagtattatcttaatgatttggagcacattcatcatagcaagctctctcctcatctcctggaggaaccggtgataactaaggatatatatgaagttgctgtctctctcattcagatgtttgatgacttggatatgaaggagagtgggaacaaggcttggtcgggggcccagtttgtgttggagcgaagcagtgtgttggtgtttttgccaggtctgggtgaaataaattatatgcatgaacttctcacaagcctggttcataaaaggttgcaggtctatccactccattcaagtgtggctttagaagaacagaataatgtctttttaagtccagtccctgggtacagaaagattattctgtccaccaatattgcagagagttctgtcacagttccagatgtcaaatatgttatagatttttgtttgactagaactttggtctgtgatgaagatacaaattatcagagtctgcgattgagttgggcttctaaaaccagctgtaatcagagaaaaggccgtgctggacgagtgtctagagggtactgttaccggctggtacacaaggatttctgggacaactccatccctgatcatgttgttcctgagatgttgcgttgtccattaggaagcacgatcttgaaagtgaaattacttgacatgggtgagccgagagctctgctggccactgccctttccccgcctggtctgagtgacattgagcgcaccatccttctactaaaggaggttggagcacttgcagtgagtgggcagagagaagatgaaaacccccatgatggtgaattgaccttcttaggaagagttttagcccaacttcctgtaaatcagcaacttggtaaactcatagtccttggacatgtatttggatgtctagatgaatgtcttattatagcggcagctctttctttgaagaatttttttgcaatgcctttccggcagcatctcgatggatataggaacaaagtgaatttctctggcagtagcaagagtgactgtattgcacttgttgaggcatttaaaacatggaaggcttgcagacagacaggggagctgcggtacccgaaggatgaacttaattggggacggttaaattacattcaaatcaagagaattagagaggtggctgaattatatgaagaattgaagactagaatctcacagttcaacatgcatgttgattctcggcgacctgtcatggaccaagagtatatatataagcagcgattcatcctacaggttgtattggcaggtgctttctatccaaattactttacttttggacagccggatgaggagatggcggtgagggagctggctggcaaggaccccaagacaactgtcgtgttgaaacacattcctccctatggatttctttactataaacaactacagtctctctttagacagtgtggtcaagtcaaatccattgtatttgatggtgcaaaagcctttgtggaattctcacgaaatccaacagagagatttaaaacccttcctgcagtatatatggcaattaagatgtctcaactaaaagtttcacttgaactcagcgttcattctgcagaggaaattgaagggaaggtgcaaggcatgaacgtctcaaagctcaggaacacaagggtgaatgtggacttccagaagcagacggtagatcctatgcaagtctcctttaacacatcagacaggtcccagacagttacagatctccttctaactattgatgtcacagaggtggttgaagtgggacacttttggggatacaggattgatgaaaacaactcagagattctgaaaaagcttactgctgaaatcaaccaactgacgctggtgcccttgcccactcacccacatccagacttggtctgtctggcaccttttgctgattttgataaacaacgctactttagagctcaagtcctttatgtttctggaaattctgctgaggtattctttgtagattatggcaataagtctcatgtagatctacatcttttgatggagattccctgtcaatttcttgaacttcctttccaggctttggaatttaagatttgcaaaatgagaccatcagcaaagtctcttgtttgtggcaagcactggagtgacggggccagccagtggttcgcctctctggtgagcggctgcaccctccttgtgaaggtcttctctgtggtgcacagcgtcctgcacgtggatgtgtaccagtactcaggggtccaggatgccatcaacataagagacgtcctcatccagcagggctatgccgagctcacggaggagtcctacgagtccaagcaaagccatgaagttctcaagggcctcttttccaagtcagtagaaaacatgacagatggctctgtgccctttcccatgaaagacgacgagaaatatctcatccggattttgttagagagcttttctaccaataaactgggtactccaaactgtaaggcagaacttcacgggccttttaacccttatgaactaaagtgccatagtttgaccagaatatccaaattcaggtgtgtttggattgagaaggagagcatcaactctgtcattatcagtgacgcccctgaagaccttcaccagagaatgctggttgcagcttccctttccatcaatgcgactggatctacgatgctgctgagagaaacctctctgatgcctcatatccctggcctcccggctctcctcagcatgttattcgcaccggtgatagagttaaggattgatcagaatggcaagtactatactggagtcctttgtggtttggggtggaatccagctacaggggcttccatactgcccgagcacgacatggagcttgcgtttgacgttcaattcagcgtggaggatgtcgtcgaggttaatattctcagggctgctattaacaagctagtctgtgatggaccaaatggatgcaagtgtcttgggccagagagagttgcgcagcttcaagacattgcccgtcagaagcttttaggtttgttctgtcagtcaaaaccaagggagaagattgttcccaagtggcatgaaaagccctacgagtggaatcaggttgatccaaagctggtcatggagcaggccgaccgtgagagcagcagagggaagaacacctttctctaccagctccacaaactggttgtgctcggcacctgagcatgtccacaggtggcctccagcacacccctcaggaagctgtggaggctggattccaggctccctccgcagactgactttcctctgtgtctgggtgttacagtctgtgcccactgcatcctaaaggccttttctttcttcttttctctttgggtgatagtcagagagtggtgtttttgttcaggtgggaaggattggaaactctagtcttttctagaaacagaaaatcactgtattaaatattttggaaagattgttctgaaagaagtctgtttggataaagagctgtattttgctttaaatttattaaggtaaatataagtagttaatcttagatgtaaggttccagaatgtgcttacatattctgttctgttacagtgatttaaaccagtagtataggaaaaaacttaaaaaacaaaaaaaccatgtagtattttctgatttttttttccatgagggaaaatatctaatttttataagactaagttgagttatacttcttggttcacattttggaaatcagagattacagattacatggccatagcttatctgtgttaaaacaataaaagcattaaatgaaaaaaaaaaaaaaaaaaaaa

SEQ ID NO: 9 - DNA, 4138 bp, Homo sapiens
gggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggctgctggtgggtgctccccaggccctggctcttcctgggcagcaggcgaatcgcactggaggcctcttcgcttgcccgttgagcctggaggagactgactgctacagagtggacatcgaccagggagctgatatgcaaaaggaaagcaaggagaaccagtggttgggagtcagtgttcggagccaggggcctgggggcaagattgttacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaaggggttgctttttgtgaccaacattgatagctcagaccccgaccagctggtgtataaaactttggaccctgctgaccggctcccaggaccagccggagacttggccctcaatagctacttaggcttctctattgactcggggaaaggtctggtgcgtgcagaagagctgagctttgtggctggagccccccgcgccaaccacaagggtgctgtggtcatcctgcgcaaggacagcgccagtcgcctggtgcccgaggttatgctgtctggggagcgcctgacctccggctttggctactcactggctgtggctgacctcaacagtgatggctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccagatattgcagtgggtgccccctttgatggtgatgggaaagtcttcatctaccatgggagcagcctgggggttgtcgccaaaccttcacaggtgctggagggcgaggctgtgggcatcaagagcttcggctactccctgtcaggcagcttggatatggatgggaaccaataccctgacctgctggtgggctccctggctgacaccgcagtgctcttcagggccagacccatcctccatgtctcccatgaggtctctattgctccacgaagcatcgacctggagcagcccaactgtgctggcggccactcggtctgtgtggacctaagggtctgtttcagctacattgcagtccccagcagctatagccctactgtggccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagagatccacttcctgaagcaaggctgtggtgaagacaagatctgccagagcaatctgcagctggtccgcgcccgcttctgtacccgggtcagcgacacggaattccaacctctgcccatggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggagaagccactctgcctgtccaatgagaatgcctcccatgttgagtgtgagctggggaaccccatgaagagaggtgcccaggtcaccttctacctcatccttagcacctccgggatcagcattgagaccacggaactggaggtagagctgctgttggccacgatcagtgagcaggagctgcatccagtctctgcacgagcccgtgtcttcattgagctgccactgtccattgcaggaatggccattccccagcaactcttcttctctggtgtggtgaggggcgagagagccatgcagtctgagcgggatgtgggcagcaaggtcaagtatgaggtcacggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggatgtggacagtagggataggaggcggcgggagctggagccacctgagcagcaggagcctggtgagcggcaggagcccagcatgtcctggtggccagtgtcctctgctgagaagaagaaaaacatcaccctggactgcgcccggggcacggccaactgtgtggtgttcagctgcccactctacagctttgaccgcgcggctgtgctgcatgtctggggccgtctctggaacagcacctttctggaggagtactcagctgtgaagtccctggaagtgattgtccgggccaacatcacagtgaagtcctccataaagaacttgatgctccgagatgcctccacagtgatcccagtgatggtatacttggaccccatggctgtggtggcagaaggagtgccctggtgggtcatcctcctggctgtactggctgggctgctggtgctagcactgctggtgctgctcctgtggaagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggctgctccttctctagtttcccctctcatctgaccttagtttgctgccatcagtctagtggtttcgtggtttcgtctatttattaaaaaatatttgagaacaaaaaaaaaaaaaaaaaa

SEQ ID NO: 10 - DNA, 2530 bp, Homo sapiens
gggaatatgagtgatggtgcctcaaagcagtaactttttgcttagagcttgagagtcaaagttaaggacccacatgtatacttcggctctagcgagtctaaggatgataatatggatacaaaatctattctagaagaacttcttctcaaaagatcacagcaaaagaagaaaatgtcaccaaataattacaaagaacggctttttgttttgaccaaaacaaacctttcctactatgaatatgacaaaatgaaaaggggcagcagaaaaggatccattgaaattaagaaaatcagatgtgtggagaaagtaaatctcgaggagcagacgcctgtagagagacagtacccatttcagattgtctataaagatgggcttctctatgtctatgcatcaaatgaagagagccgaagtcagtggttgaaagcattacaaaaagagataaggggtaacccccacctgctggtcaagtaccatagtgggttcttcgtggacgggaagttcctgtgttgccagcagagctgtaaagcagccccaggatgtaccctctgggaagcatatgctaatctgcatactgcagtcaatgaagagaaacacagagttcccaccttcccagacagagtgctgaagatacctcgggcagttcctgttctcaaaatggatgcaccatcttcaagtaccactctagcccaatatgacaacgaatcaaagaaaaactatggctcccagccaccatcttcaagtaccagtctagcgcaatatgacagcaactcaaagaaaatctatggctcccagccaaacttcaacatgcagtatattccaagggaagacttccctgactggtggcaagtaagaaaactgaaaagtagcagcagcagtgaagatgttgcaagcagtaaccaaaaagaaagaaatgtgaatcacaccacctcaaagatttcatgggaattccctgagtcaagttcatctgaagaagaggaaaacctggatgattatgactggtttgctggtaacatctccagatcacaatctgaacagttactcagacaaaagggaaaagaaggagcatttatggttagaaattcgagccaagtgggaatgtacacagtgtccttatttagtaaggctgtgaatgataaaaaaggaactgtcaaacattaccacgtgcatacaaatgctgagaacaaattatacctggcagaaaactactgttttgattccattccaaagcttattcattatcatcaacacaattcagcaggcatgatcacacggctccgccaccctgtgtcaacaaaggccaacaaggtccccgactctgtgtccctgggaaatggaatctgggaactgaaaagagaagagattaccttgttgaaggagctgggaagtggccagtttggagtggtccagctgggcaagtggaaggggcagtatgatgttgctgttaagatgatcaaggagggctccatgtcagaagatgaattctttcaggaggcccagactatgatgaaactcagccatcccaagctggttaaattctatggagtgtgttcaaaggaataccccatatacatagtgactgaatatataagcaatggctgcttgctgaattacctgaggagtcacggaaaaggacttgaaccttcccagctcttagaaatgtgctacgatgtctgtgaaggcatggccttcttggagagtcaccaattcatacaccgggacttggctgctcgtaactgcttggtggacagagatctctgtgtgaaagtatctgactttggaatgacaaggtatgttcttgatgaccagtatgtcagttcagtcggaacaaagtttccagtcaagtggtcagctccagaggtgtttcattacttcaaatacagcagcaagtcagacgtatgggcatttgggatcctgatgtgggaggtgttcagcctggggaagcagccctatgacttgtatgacaactcccaggtggttctgaaggtctcccagggccacaggctttaccggccccacctggcatcggacaccatctaccagatcatgtacagctgctggcacgagcttccagaaaagcgtcccacatttcagcaactcctgtcttccattgaaccacttcgggaaaaagacaagcattgaagaagaaattaggagtgctgataagaatgaatatagatgctggccagcattttcattcattttaaggaaagtagcaaggcataatgtaatttagctagtttttaatagtgttctctgtattgtctattatttagaaatgaacaaggcaggaaacaaaagattcccttgaaatttagatcaaattagtaattttgtttatgctgctcctgatataacactttccagcctatagcagaagcacattttcagactgcaatatagagactgtgttcatgtgtaaagactgagcagaactgaaaaattacttattggatattcattcttttctttatattgtcattgtcacaacaattaaatatactaccaagtacagaaatgtggaaaaaaaaaa

SEQ ID NO: 11 - DNA, 1478 bp, Homo sapiens
agcataaaaagaccagcagatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccataaaaaaaaaaaaaaaaaaaaaaaaaaaaaa

SEQ ID NO: 12 - DNA, 1439 bp, Homo sapiens
tgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 13 - DNA, 2613 bp, Homo sapiens
gggctgcccgggcctcactcgggccccgcggccgcctttataaggcggcgggggtggtggcccgggccgcgttgcgctcccgccactccgcgcccgctatcctggctccgtgctcccacgcgcttgtgcctggacggaccctcgccagtgctctgcgcaggattggaacatcagttaacatctgaccactgccagcccaccccctcccacccacgtcgattgcatctctgggctccagggataaagcaggtcttggggtgcaccatgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgcgctggccctctaccccctgagcgtcctgttctgagggcccagggcccgggcacccacaagcccgtgacagatgccaacttcccacacggcagcccccccctcaaggggcagggaggtgggggcctcctcagcctctgcaactgcaagaaaggggacccaagaaaccaaagtctgccgcccacctcgctcccctctggaatcttccccaagggccaaacccacttctggcctccagcctttgctccctccccgctgccctttggccaacagggtagatttctcttgggcaggcagagagtacagactgcagacattctcaaagcctcttatttttctagcgaacgtatttctccagacccagaggccctgaagcctccgtggaacattctggatctgaccctcccagtctcatctcctgaccctcccactcccatctccttacctctggaaccccccaggccctacaatgctcatgtccctgtccccaggcccagccctccttcaggggagttgaggtctttctcctcaggacaaggccttgctcactcactcactccaagaccaccagggtcccaggaagccggtgcctgggtggccatcctacccagcgtggcccaggccgggaagagccacctggcagggctcacactcctgggctctgaacacacacgccagctcctctctgaagcgactctcctgtttggaacggcaaaaaaaaatttttttttctctttttggtggtggttaaaagggaacacaaaacatttaaataaaactttccaaatatttccgaggacaaaaaaaaaaa

SEQ ID NO: 14 - DNA, 5240 bp, Homo sapiens
atctttgatcaatgtacttgccagggagagcccaagtccttcaaacctcctccttttcaccttcatccttaactttgtgctagagcgagacccacacaacaacagccgaccctccccgccccacccccacccccaaaccagccctcgatcccagcccccggagaggactcgcatttcgacttgcgggacacttttgtgcgttcctctccagagcgcctctcgtgctcgcccctcttgcgctcgctctttattaccttcacctccttttctcccccttctctccctttctccttctcgttctctcccggagttgttgttgcccccctcgctccttctccccccttttttccccttcccctcccgggggtgtgtggcaacttttcctctcgcttctcctccgtctgtttccccttatatgtgaccatggcagtgccggcggctttgatccctccgacccagctggtcccccctcaacccccaatctccacgtctgcttcctcctctggcaccaccacctccacctcttcggcgacttcgtctccggctccttccatcggacccccggcgtcctctgggccaactctgttccgcccggagcccatcgcttcggcggcggcggcggcggccacagtcacctctaccggcggcggcggcggcggcggcggcagcggaggcggcggcggcagcagcggcaacggaggcggcggtggcggcggcggcggtggcagcaactgcaaccccaacctggcggccgcgagcaacggcagcggcggcggcggcggcggcatcagcgctggcggcggcgtcgcttccagcacccccatcaacgccagcaccggcagcagcagcagcagcagtagcagcagcagcagcagcagcagtagtagcagcagcagcagtagcagcagcagctgcggccccctccccgggaaacccgtgtactcaaccccgtccccagtggaaaacacccctcagaataatgagtgcaaaatggtggatctgaggggggccaaagtggcttccttcacggtggagggctgcgagctgatctgcctgccccaggctttcgacctgttcctgaagcacttggtggggggcttgcatacggtctacaccaagctgaagcggctggagatcacgccggtggtgtgcaatgtggaacaagttcgcatcctgaggggactgggcgccatccagccaggagtgaaccgctgcaaactcatctccaggaaggacttcgagaccctctacaatgactgcaccaacgcaagttctagacctggaaggcctcctaagaggactcaaagtgtcacctccccagagaactctcacatcatgccgcattctgtccctggtctcatgtctcctgggataattccaccaacaggtctgacagcagccgctgcagcagctgctgctgctaccaatgcagctattgctgaagcaatgaaggtgaaaaaaatcaaattagaagccatgagcaactatcatgccagtaataaccaacatggagcagactctgaaaacggggacatgaattcaagtgtcggactggaacttccttttatgatgatgccccaccctctaattcctgtcagcctacctccagcatctgtcaccatggcaatgagccagatgaaccacctcagcaccattgcaaatatggcagcagcagcacaagttcagagtcccccatccagagttgagacatcagttattaaggagcgtgttcctgatagcccctcacctgccccctctctggaggaggggagaaggcctggcagtcacccatcatcacatcgcagcagcagcgtgtccagctcccctgctcggactgagagctcttctgacagaatcccggtccatcagaatgggttgtccatgaaccagatgctgatgggcttatcaccaaatgtacttcctgggcccaaagagggagatttggccggtcatgacatgggacatgagtcaaaaaggatgcatattgaaaaagatgagaccccgctttctacaccaaccgcaagagacagccttgacaaactctctctaactgggcatggacaaccactgcctccaggttttccatctccttttctgtttcctgatggactgtcttccatcgagactcttctgactaacatacaggggctgttgaaagttgccatagataatgccagagctcaagagaaacaggtccaactggaaaaaactgagctgaagatggattttttaagggaaagagaactaagggaaacacttgagaagcagttggctatggaacaaaagaatagagccatagttcaaaagaggctaaagaaggagaagaaggcaaagagaaaattgcaggaagcacttgagtttgagacgaaacggcgtgaacaagcagaacagacgctaaaacaggcagcttcaacagatagtctcagggtcttaaatgactctctgaccccagagatagaggctgaccgcagtggcggcagaacagatgctgaaaggacaatacaagatggaagactgtatttgaaaactactgtcatgtactgaatctttcctgttgaagaaatccatgttatagaaaagaactttgcagtcagacattcgtcatgggaaagttcagaaaaaaataaagtccttttaagggaacttcctgaattttgtgtattaatgttctttaaaagtttaagtattctacaaaaaaaaaaaaagttttctccattgattttcacctgtggttcataccagagacctgagaatgtttgtaaatgtacaagtatcaaagttcttacagttaattactgcaacttgctgctggacaattgtatacagagttaaaggcaggtctgaataagacctagctttgtttttttctaatggaatgaaccattttcctcttctgaaaattctgtatctgagcacatcaagagactcttgtagcagtggttacccagacttacagaattatgtcctccagaaaccagcaagaacacttggaatgaacgaatgaacttgtagggggcatagaggattcttgaaaaaaaaaaatgcaagagtgattttctgttacattcaatttcaaactctctaattgtgggttttctcctgaagaattttttttcacatactttccaaaagaccaacaaatggatgttgacaacaacccaatgaaataacattttgcatatctgaaaagaagcattgaatataagccaaaagctttcactgaaggtttttttttcttaaaaataaaaaaaaatatataagtgtaacatgttttcattccaaactggtagtggtatatagaattaaagataataatgttgcttcttattcaaactgttggtcatatgtacagtatataaacataaaacacacaaggaaggtattatgtatgcagtagtatactagagtttaggaaaatgaaaattttagaaaatatgttttgtcaccctgttggtcagaaagatgtctttctggttttaacgcatgcaggcatgtaaatatttgtctggagtcacagtattaatgaatgagatcttaagcatctggtgacatcagaactctgtgtcagccacttttatttgtatattgaaccctagctagtgccccaagctgcactattgggaatggattgtggctgaacagcaaatcaaaacaccagaaatatttttatatgttaacgtcatattatgttaatgttgctgaaaacaaaacctaacaaaccttgatgtaccagtccaataccatgtagcgctgagtgataaagttaaaatgtgctgtgcttcccacccttgtcagagggaagggtggctatgtgttattttcactgtctttttgaaagttacagtatgtgttttcactttcgtgcagataactggaagtaaagcggcaaacagtgcttattacatgctaaagttaccttctctttgttttttgcatatctggaattacacctttaaagactgatatgaatcagtacggtcactatacattttatgatttttctgtcatcttaaaattgtatgatcgtaacattatttattaccacaaaacagcaaaatcttcaatgtctaagaaaactagcttaaaatgtttaaatatagttctgattgggtattaattacttgattaagaaaaaattaacattatagatactctggcattacgcttctataccttttaggtcttccttgcaatactggaacataattcttttgtgtagctcactattagccagctaagttcatctttttaataccataaaaaggttatatgtacagttcctattttagcttgcttacaaagggagcattatttttatttaaagtattgctagtaaatgatttgtagaaacttggttttctaagcatagttcttccataaccaccttttgttgtttgagcacaagggattcttttcctagttctatgtgtttgtttccctatatgcagtctttaaaggattacaacacttaaaattgaatggacttgtgtcaagctttttgcatcatacattttttgaaagatttttaaaaaagcctacaacttacatatgtagtagaatcagccattgctctgctcctggcatagagtcacctgtttgttatgtggattaaatagttttaaaatacatatttgaagacctttgagaatgctttagtgtttgatttgaaataaaaggaaattttagcaaggattaaagaaaaaagctatcagctgtatgttaagagagactcttactaacatgttgtaaatattacaattcatgaaatgttattgtaagtctgtaacttaattttttccctgttttagttatacaggttggtttggaaatttgtgttttggcataaacaagtaaaatgtgcccattttatggtttccatgcttttgtaatcctaaaaatattaatgtctagttgttctatattataaccacatttgcgctctatgcaagcccttggaacagaacatactcatcttcatgtaggacctatgaaaattgtctatttttatctatatatttaaagttttctaaaaatgataaaaggttattacgaattttgttgtacaaaatctgtacaaaaatctgtttttacatcataatgcaagaattggaaatttttctatggtagcctagttatttgagcctggtttcaatgtgagaaccacgtttactgttattgtatttaattttcttttccttttcaacaatctcctaataaaactgtctgaaatctcaaaaaaa

SEQ ID NO: 15 - DNA, 5170 bp, Homo sapiens
gtggccggcggccggagccgactcggagcgcgcggcgccggccgggaggagccggagagcggccgggccgggcggtgggggcgccggcctgccccgcgcgccccagggagcggcaggaatgtgacaatcgcgcgcccgcgcaccgaagcactcctcgctcggctcctagggctctcgcccctctgagctgagccgggttccgcccggggctgggatcccatcaccctccacggccgtccgtccaggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaaaaaaaaaaaaaaaaa

SEQ ID NO: 16 - DNA, 1675 bp, Homo sapiens
acctcatttcctgtgtcctctctttctttggcaatccaaagaaagtcatctttcagattgtctgctcagagttcatctcaaagcctggcaaggattggagaggtcaataagagtcagcgcctttaaaaagaaatctactcactcttctgtgtgcataaggccgagcagaggttcttcgtctcaagaggaactgacttctgttgagcactcaacacgccacagagaccagccatcttgcaacctcacctcacagcatggagagaggagaccaacctaagagaaccaggaatgaaaacattttcaactgcttatacaaaaaccctgaggcaacttttaagctgatttgctttccctggatgggaggtggctccactcattttgccaaatggggccaagatactcatgatttgctggaagtgcactccttaaggcttcctggaagagaaagcagagttgaagaacctcttgaaaatgacatctcccagttagttgatgaagttgtttgtgctctgcagccagtcatccaggataaaccatttgcattttttggccacagtatgggatcctacattgcttttaggactgcactaggtctaaaagaaaacaatcaaccagaaccattgcatttatttttgtcaagtgcaactcctgtacattcaaaggcctggcatcgcattcccaaagatgatgaattgtcagaagaacaaataagtcattaccttatggaatttggaggcacccccaagcattttgctgaagccaaggaatttgtgaaacaatgtagtcccatcataagggcagatctgaacattgttagaagttgcacctctaacgtaccatctaaggctgttctttcctgtgacttgacatgttttgttggatctgaagacatagcaaaggacatggaagcctggaaagatgtaaccagtggaaatgctaaaatttaccagcttccagggggtcacttttatcttctggatcctgcgaacgagaaattaatcaagaactacataatcaagtgtctagaagtatcatcgatatccaatttttagatattttccctttcacttttaaaataatcaaagtaatatcatactcttctcagttattcagatatagctcagttttattcagattggaaattacacattttctactgtcagggagattcgttacataaatatatttacgtatctggggacaaaggtcaagccagtaaagaatacttctggcagcactttgggaggccaaggcgggcggatcacgaggtcaggagatcgagaccgtcctggctaacaccgtgaaaccccatctctactaaaaatacacaaaattagccgggcgtggtggtgggcacctgtagtcccagctactcgggaggctgaggcaggagaatggtgtgaacctgggaggtggagcttgcagtgaaccgagatcgctccactgcactccagcctgggtgacagatccagactctgtctcaaaaaaaaaaaaaaaaaaaatacttctggcagagtcttttatcttcctattaaaatctcacttgattctcctttatgggaagtttgtcgacaaaattcatgattagtaaattatccattttttccttcagttagtttaatggtgaagatgattaacaggggaaatgcttgaagtaaatgattgtttcaatggc

SEQ ID NO: 17 - DNA, 1484 bp, Homo sapiens
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 18 - DNA, 4531 bp, Homo sapiens
gggagtttctggagggctgaacacgtggaggcaaacaggaaggtgaagaagaacttatcctatcaggacggaaggtcctgtgctcgggatcttccagacgtcgcgcggtgtcagaaaccctgtggtgaaattcagcctgtggattccagaaatttggagtgttcttggggggaaaaatccgcacacacaaagcaacatttggaaatccctgtggactctaaattgccccctctgaggtcaaggaacacaagatggttttggaaatgctgaacccgatacattataacatcaccagcatcgtgcctgaagccatgcctgctgccaccatgccagtcctgctcctcactggcctttttctcttggtgtggaattatgagggcacatcctcaataccaggtcctggctactgcatgggaattggacccctcatctcccacggcagattcctgtggatggggatcggcagtgcctgcaactactacaaccgggtatatggagaattcatgcgagtctggatctctggagaggaaacactcattatcagcaagtcctcaagtatgttccacataatgaagcacaatcattacagctctcgattcggcagcaaacttgggctgcagtgcatcggtatgcatgagaaaggcatcatatttaacaacaatccagagctctggaaaacaactcgacccttctttatgaaagctctgtcaggccccggccttgttcgtatggtcacagtctgtgctgaatccctcaaaacacatctggacaggttggaggaggtgaccaatgaatcgggctatgtggacgtgttgacccttctgcgtcgtgtcatgctggacacctctaacacgctcttcttgaggatccctttggacgaaagtgctatcgtggttaaaatccaaggttattttgatgcatggcaagctctcctcatcaaaccagacatcttctttaagatttcttggctatacaaaaagtatgagaagtctgtcaaggatttgaaagatgccatagaagttctgatagcagaaaaaagacgcaggatttccacagaagagaaactggaagaatgtatggactttgccactgagttgattttagcagagaaacgtggtgacctgacaagagagaatgtgaaccagtgcatattggaaatgctgatcgcagctcctgacaccatgtctgtctctttgttcttcatgctatttctcattgcaaagcaccctaatgttgaagaggcaataataaaggaaatccagactgttattggtgagagagacataaagattgatgatatacaaaaattaaaagtgatggaaaacttcatttatgagagcatgcggtaccagcctgtcgtggacttggtcatgcgcaaagccttagaagatgatgtaatcgatggctacccagtgaaaaaggggacaaacattatcctgaatattggaaggatgcacagactcgagtttttccccaaacccaatgaatttactcttgaaaattttgcaaagaatgttccttataggtactttcagccatttggctttgggccccgtggctgtgcaggaaagtacatcgccatggtgatgatgaaagccatcctcgttacacttctgagacgattccacgtgaagacattgcaaggacagtgtgttgagagcatacagaagatacacgacttgtccttgcacccagatgagactaaaaacatgctggaaatgatctttaccccaagaaactcagacaggtgtctggaacactagagaaggctggtcagtacccactctggagcatttctcatcagtagttcacatacaaatcatccatccttgccaatagtgtcatcctcacagtgaacactcagtggcccatggcattttataggcatacctcctatgggttgtcaccaagctaggtgctatttgtcatctgctcctgttcacaccagagaaccaggctacaagagaaaaagcagaggccaagagtttgagggagaaatagtcggtgaagaaaccgtatccataaagacccgattccaccaaatgtgctttgagaaggataggccttcattaacaaaatgtatgtctggttccccagtagagctctactgcctcaacccaaggggatttttatgtctggggcagaaacactcaagttgattagaaagaccaggccaatgtcagggtacctggggccaaacccacctgctagtgtgaattaaagtactttaattttgttttctgtggaggtggaaaagcaacattcatagtctttggagaaatgcttagaaattcagcatttgacccttgctgtgaattaagcccaattaattcctgtttgtctacatatgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaactttccaaacaccactttacttgtgttatatagccaatataactatctctactgaatgtcattgaaaaactaaaaaattaaacttatttacaaataggtaaatatttgtcattgaatccattgccatcccatttgactgttcttttcatcctactgtctagtaataagctgagtataagatgacagtgtaatctccctgaaagcaggagctactttctttcttttgtaatctatttccatccccatttccctgtcctgtctccctgtattcactcccaagctcagttctgaatagacattcctgctcagagatactcccaactgatgcagaaaccaaataaagaggtaggtattccaagaattcaagaatggacattagtaaagaataaaacatttatttgagcttggaattatttggatcatctatatggcctaaaaatatatggactatgcctgtgtacctgaatacgtatgtagtcaggtcaagacaatcatccaaataacttagacccctaaaagcaaggccaggatttgcaatttaatgtgtcccaattaattcacttgaaaattagtaacactctgtttacgttgcctctggctggagctgcatggtggaagaagcccaactttggatccatgtacttcacccatccaatactcttgggacatttatgtgtattttatctgtatatatgaagccaatgtctatgtctacacagtcaaagtgaaatgcatgtttgatatagctgtacatagatatctattttgcaggtacaaaaatatcctgggggaaaactgggagtggaagggtggggggtgggagtgagggacatgggggagggacaggaagaggagaagtgttggtttgaacgatccaagcaaactctcccagaatcaaattacctgggtagttgttcaacttttcactctgcttagcctgtatagacaaaccccatatatttgtagaggcttggccttggaattctggaataccattggcttttcagtaggctgatgaacacattttgaaaattctattatcttcagaattttgccccattgttaagtgcttaaccgtcactcttgaatgtgcaatgtgctgtggattccattttcatcagttctgaaagaactgcaatgtgtaaattatcagtgaaatgcatgcatataagggctctatcattatcaaattgtaaggacaattgtacccttctatatctttgggcatgctagacacccccatgccttcattgagatcccattttccccctctcaagtggaaaataatcacatccagcaagctctctcattattgagaaataccatttggaaattgccactttttattcctaagcagcacctttcactgttcatgatgctaatgttccacaaaagcatgtgccattggcccactgaaggatagagggacccttttcaatctatatcagctgggctctgggactgaatctctcacctattcttgcagaaagacatactaattaaaccttgtcaaagtaaaaaaaaaaaaaaaaaa

SEQ ID NO: 19 - DNA, 3056 bp, Homo sapiens
gacacatgatgctgtgaacgtcagggtgctcgccagggaagggccctacccagagggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcaagcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagaggcatagtcacctaggggaggaggccgtatgaagacagaggcagagattggagtgacgcatctccaagccaaggaattccaaggactgtaagccaccagtagaagctttgaagaggcaaggaaggattccctccaatagccttcaagtgtgaccctgctgacacctgcagaattcggacttctatcctccaaaaccgtgagggaataaatttcctttgttttaagccaccaactttgcaatactttgttacagcaaccctagacatgaggtactagacacagtacatctacacatatgaaaatgaatcaacacagaatgcagaagtagaacccttgctaaggactactgggcatcttcccaggacagcagccaaaagagaaccaccacttcctctcctgcctcctccttgctctctcctagagtccaaacccaaatgggccagttggatctgatgttcgtcagttctttacttctatttcctggggtactcaggagggcacacactatagataacttgggttagctgcataaaattcaatgtctcattaagttgcattaaactgagcttagatgtgtaagtttgctaacggatgggtttttttgttaagaactataggatttatgggaccaagtctagcgagtccagatatcaaaatcattataatgttatatttgctgttattagaatataatatagcttattatacaataaatatgtagactgtaaaatatatttctcactagtacctcctattttctttctctgttgaagtttttaaatcccacagataattaaattggcacctttatgcttgttcaaaaattaaaataatctattaaataagttcaaattaaagatttttacttcaaatgac

SEQ ID NO: 20 - DNA, 4326 bp, Homo sapiens
gtcagctgtgccccggtcgccgagtggcgaggaggtgacggtagccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaatcaaaaaaaaaaaaaaaaaa

SEQ ID NO: 21 - DNA, 1034 bp, Homo sapiens
gtgagaaggaaactgcaagagtggggcagagaaccagagtgtcagagcaaaacctcctctatctgcacatcctggggacgaaccgggcagccggagagctgcggccggcccagtcccgctccgcctttgaagggtaaaacccaaggcggggccttggttctggcagaagggacgctatgaccgcagaattcctctccctgctttgcctcgggctgtgtctgggctacgaagatgagaaaaagaatgagaaaccgcccaagccctccctccacgcctggcccagctcggtggttgaagccgagagcaatgtgaccctgaagtgtcaggctcattcccagaatgtgacatttgtgctgcgcaaggtgaacgactctgggtacaagcaggaacagagctcggcagaaaacgaagctgaattccccttcacggacctgaagcctaaggatgctgggaggtacttttgtgcctacaagacaacagcctcccatgagtggtcagaaagcagtgaacacttgcagctggtggtcacagataaacacgatgaacttgaagctccctcaatgaaaacagacaccagaaccatctttgtcgccatcttcagctgcatctccatccttctcctcttcctctcagtcttcatcatctacagatgcagccagcacagttcatcatctgaggaatccaccaagagaaccagccattccaaacttccggagcaggaggctgccgaggcagatttatccaatatggaaagggtatctctctcgacggcagacccccaaggagtgacctatgctgagctaagcaccagcgccctgtctgaggcagcttcagacaccacccaggagcccccaggatctcatgaatatgcggcactgaaagtgtagcaagaagacagccctggccactaaaggaggggggatcgtgctggccaaggttatcggaaatctggagatgcagatactgtgtttccttgctcttcgtccatatcaataaaattaagtttctcgtcttaaaaagaaaaaaaaaaaaaa

SEQ ID NO: 22 - DNA, 1198 bp, Homo sapiens
tactaagagtctccagcatcctccacctgtctaccaccgagcatgggcctatatttgaagccttagatctctccagcacagtaagcaccaggagtccatgaagaagatggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgcaatatacatagaaacgtctgtgctcaaggatttatagaaatgcttcattaaactgagtgaaactggttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtaaaaaaaaaaaaaaaaaaaaaaaaaaa

SEQ ID NO: 23 - DNA, 1606 bp, Homo sapiens
actaagtatctccactttcaattctagatcaggaactgaggacatatctaaattttctagttttatagaaggcttttatccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtaaagatgatcaaccatctcaataaaagccaggaacagagaagagattacaccagcggtaacactgccaactgagactaaaggaaacaaacaaaaacaggacaaaatgaccaaagactgtcagatttcttagactccacaggaccaaaccatagaacaatttcactgcaaacatgcatgattctccaagacaaaagaagagagatcctaaaggcaattcagatatccccaaggctgcctctcccaccacaagcccagagtggatgggctgggggaggggtgctgttttaatttctaaaggtaggaccaacacccaggggatcagtgaaggaagagaaggccagcagatcactgagagtgcaaccccaccctccacaggaaattgcctcatgggcagggccacagcagagagacacagcatgggcagtgccttccctgcctgtgggggtcatgctgccacttttaatgggtcctccacccaacggggtcagggaggtggtgctgccccagtgggccatgattatcttaaaggcattattctccagccttaagtaagatcttaggacgtttcctttgctatgatttgtacttgcttgagtcccatgactgtttctcttcctctctttcttccttttggaatagtaatatccatcctatgtttgtcccactattgtattttggaagcacataacttgtttggtttcacaggttcacagttaagaaggaattttgcctctgaataaatagaatcttgagtctcatgcaaaaaaaaaaaaaaaaaa

SEQ ID NO: 24 - DNA, 740 bp, Homo sapiens
gcctcacagaattgagagtttgttcttacacacaagtttaatgccaccttcctctgtctgccatggaccaacaagcaatatatgctgagttaaacttacccacagactcaggcccagaaagttcttcaccttcatctcttcctcgggatgtctgtcagggttcaccttggcatcaatttgccctgaaacttagctgtgctgggattattctccttgtcttggttgttactgggttgagtgtttcagtgacatccttaatacagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtggataaacggctcttttttaaattctaatgacttagaaattagaggtgatgctaaagaaaacagctgtatttccatctcacagacatctgtgtattctgagtactgtagtacagaaatcagatggatctgccaaaaagaactaacacctgtgagaaataaagtgtatcctgactcttga

SEQ ID NO: 25 - DNA, 6252 bp, Homo sapiens
gggcgggggaaggagcatctcaggaaaggggtccccggactctggggctctcagcacctgcggtcgcaaaccaacctcatgccctgactttaccaggcgtcgggactctgacttaaccggggaatgagggacttggtctggcggcagatcacaatgaggacctagggcatctgtctgctgacgccccctggcctgcagtgaccatggccccccgcaagaggagccaccatggcctgggcttcctgtgctgcttcgggggcagtgacatccccgaaatcaacctccgggacaaccaccctctgcagttcatggagttctccagccccatcccgaacgcagaggagctcaacatccgctttgcagagctggtggatgaattggatctcactgacaaaaaccgagaggctatgtttgcactgccccctgagaagaaatggcagatctactgcagcaagaagaaggagcaggaggaccccaacaagctggcaaccagctggcctgactattacatcgaccgcatcaattccatggctgcgatgcagagtctgtacgcgtttgatgaggaggagacggagatgaggaaccaagtcgtggaagacctgaagacagccctccggacacagcctatgaggtttgtgacccgcttcattgagctggagggcttgacctgtctgctaaatttcctccggagcatggaccacgccacctgtgagagccgcatccacacctcactcattggctgcatcaaagcattgatgaacaactcccaggggcgggcacatgtgctggcacagcctgaggccattagtaccatagcccagagcctacgcacagagaacagcaagaccaaggtggctgtgctggagatcctgggtgctgtgtgcctcgtgcctggtggccacaagaaggtgctgcaggccatgctgcactaccaggtgtatgcagcagagcgaacccgcttccagaccctgctgaacgagctagaccgaagtctgggccggtaccgggatgaagtgaatctgaaaacagccatcatgtccttcatcaatgctgtcctcaatgctggagctggagaggataatctggagttccgcctacatctacggtatgaattcctgatgctgggtatacagcctgtgattgacaagctccggcaacatgaaaatgccatcctggacaaacatttagacttcttcgagatggtgcggaatgaggatgacctggagctagccaggaggtttgacatggtccacatcgacaccaagagtgcttcccagatgtttgagttgatccacaagaagctgaagtacacggaggcctacccctgcctgctctctgtgctgcaccactgcctgcagatgccctacaaacggaacggtggctacttccagcagtggcagctcctggaccgcatcctccagcagattgtcctccaggatgagcggggtgtggaccctgacctggctcccttggagaacttcaatgtcaagaacatcgtcaacatgctcatcaacgagaatgaagtgaaacagtggcgagaccaggcagagaagttccggaaagaacacatggagcttgtgagccgtctggagaggaaggagcgggaatgcgagacaaagacattggagaaggaagagatgatgcggacgctgaacaaaatgaaggacaagctggcccgggagtcccaggagctgcgccaggctcggggacaagtggcagagctggtagcccagctcagtgaactctcaacaggccctgtatcttccccaccaccccctgggggcccactcaccttgtcttcctcaatgacaaccaatgacctgcctccaccccctcctcctctgccctttgcctgttgtccccctcccccaccaccaccccttcctcccgggggacccccgactcccccaggtgccccaccttgcctcggcatgggcctgcccctccctcaggacccctaccccagcagtgacgtcccactcaggaaaaagcgtgtcccccagccttctcacccactgaagtccttcaactgggtgaagctgaatgaggagcgtgtccctggcaccgtatggaatgagattgatgacatgcaggtatttcggatcctggacctagaggattttgaaaagatgttttcagcctaccagaggcaccagaaagagctgggctccactgaagacatctacctggcttcccgcaaggtcaaagagctgtcggtcattgatggccggagggcccaaaactgcatcatccttctttccaagttgaagctttctaacgaggagatccggcaggccatcttgaagatggatgagcaggaggaccttgctaaggacatgctggagcagctcctcaagttcatcccagagaagagtgacattgacctcctggaggagcacaagcatgaaattgagcggatggcccgtgctgaccgcttcctctatgaaatgagcaggattgaccactaccagcagcgactgcaagccctcttcttcaagaagaaattccaggagcggctggctgaggcaaagcccaaagtggaagccatcctgttggcctcccgggagctggtccgcagcaagcgtcttagacagatgctagaggtcatcctagccataggcaacttcatgaacaaagggcagcgtgggggcgcctacgggttccgggtggccagcctcaacaagatcgctgacaccaagtccagcatcgacagaaacatctctctgctccattacctgatcatgatcctggagaagcattttcctgatattctaaacatgccttcagagctgcaacatcttccagaagctgccaaagtcaacctagcagaactggagaaggaggtgggcaacctcaggaggggcctgagagcggtggaggtgctggagtatcagaggcgccaggtacgggagcccagtgacaagtttgtccctgtcatgagcgacttcatcacggtgtccagcttcagcttctccgagctggaggaccagctaaatgaggccagggacaagttcgccaaggccttgatgcacttcggggagcatgacagcaagatgcagccagacgaattctttggcatctttgataccttcttgcaggccttctcagaggcccggcaggatctagaggccatgaggaggaggaaggaggaggaggagcggcgggcgcgcatggaagccatgctgaaggagcagagggaacgtgagcggtggcagcggcagcggaaggtcctggctgcaggcagctcgctggaggagggaggagagttcgatgacctggtgtcggccctgcgctctggggaggtcttcgacaaggacttatgcaagctcaagcgcagccgcaagcgatcagggagccaggccctggaagttacccgggagcgggcaataaaccggctaaattattgacctggggaactagccacacaggaggccgggagacagggactggtgagaatggggctgagtggaggaggtggtgatatttaaaccatttggtgcttggtttagagccttgggctgggtcctgggatggggggctgtgtgtggctggaccaggtgtctccccacgcttaccttaaggggctcctcttatctccccttcacatgattccttctgtgccctggccccaggtattattctgaggctgccttggatggcctcaggccaggtaaccccaggctgaaggggccctgctccccatcccctaccatgggcacccatgtgctggcacagaacagttccagatctagactggagaggtccacagccttgtccagagttcctgtgtagcacggggagcaatgatggagggagcccctgagagggaatctggtgagggaatccagactcccttctctcaaggggaggctcaacagaacattgacctgggggcaaactttcctcttgaatgggaacagaggaggcattatatattctagttagatcagctctggtaggttccagagaacagtcaatgttggaaggatgatgcagggaccaaagccatcaggacagagtagcagtgtctgtttcccatgtcacaagtcctctggcctctccctgcatgtcttaagtatctttcccttccttctctaccctcacctccatcctgtctactaatccacagtcctagaagactcaccttgggtttccacagctatggctcactaccaggtgcttgatgaatctggcgaggggctcaagacagacctcatgcatcaccacacctcatgccttttgggcatctcccatgtccccatctcctggacacctggccattgttgtgaagccagacagtgacctcaaatgttgccttggagtcccctacagcccctcagcagagggcagcacttgaatgcttagctccatcccatagttctctacttcatataaattgctcaggccctcccaccccttctctaacactagcttcaaggcagaagccacagcagcctctgtccagcctgcaggtggccacttggaaccatgtgtccactggcgttggggagttggttcctgagaggtctgagggccagagctgccctctacattaacatgctgtctctaagggtggcccctcctctcaggcgttcagatggtgcgaacagcagagcaggcaagggaaactggggagatggggatggaggaggaaggctgatatcctctggggagcacatcacctgaaggtgccaaggaggaaggctgagaggggggccaccccatttctggtacccaatttggttcttcagcccaacttgcaaggggttccttctggtcctcccatccactgccaccttccattttgtccatctcatgctggccttggtggatgggatggctgtatctagacaaaatttttctaaaactccatcaaggctcttattcaataccacgttccgagttggcctttcatcttctttgagactggccctgcctaacctctaccatcaatgagctcttggcccttctgcccttccctgtgtttctcactttccaacctaatccctggctcagggttattgccagtggagactggtgagctgggcctactctcagctgcctatcttctgcctttcacttgcatccaactcctggggctgggaccgtagtagctgcgggggggaagaaacacagggtcggtgagcccagcatgtgcgttggtttgagggggcgggcggtgtgtgtgtgttctggtgggagggatctgagcaagtgcaagcctggctgacacaggtgtgaagaggccatcctggaacccaggtgagggcaagatgaaggcttccaggcagaacagctgcagagagtttggctatatgcatctgcagccccaagagctcccactgcaagacaagtgttggggaagatgggaggttgtgggtgaggcctctaaaggtcctctcccaaactgaccaggctgatgtcaacctaaccccctcaggggcagggaacaggggagggctccacaagcgtgtctggcattcccacccaccatggaagactggatacgcacctggaaacaaaaggactatggaagctgttcaagatacatttgatcttcagaaaagcagaatttggttcaactgttgacagaggacacaaatacgttgttccagagctcagccttctcactctaaaagaaagatatttttctatttattttctacatctggccagtggctctggtgctagatgccactgtagccagatctccaacagtgccttggaccatggactcatactcaactgagtaagaaggggctggtgcccagtcggggtggctgagctggtccttaataggttgtttcttggtcttgctttcttcatgccctccccactgctcctgccacctttagataagtttctctagctaattttgtggccaatgtaaaattcgtcatcaacctaacaaacacaaccttctcagcagcatttctcccctgtgatggaaataaagtgtttagggcagtgggaggagaaaattctccaggtgaatggggaagggtctgttccagcctctccctactcccatcccatttccaccaactggggaactgtgactatctatctcccccgacttctaccagggatgccttcacgccaaggctgttctcaccagctgcctcagatgacaaatgaggctaatggacataatctacagtgtcctttttcacttgcaccttttttataagaatatattgtaatactaaaaaatattaaattcataccatccctacccagtctgcctaaaaa

SEQ ID NO: 26 - DNA, 1312 bp, Homo sapiens
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 27 - DNA, 8044 bp, Homo sapiens
tgcgctttccacctaccagaccctgaaagaaagtgtcaggagccggtgcaaaacccagtttaagttcaagaagacatttgcaagtgcaagaggccaagcagtttgaagaagtgtaagagattttttttccttcgaaagaatatatttttaaagaaaccagccagtccgcggaaagcaacagcagttttttttttttttgcctctttttcttattttagatcgagaggtttttcttgcttttcttcccttttttttctttttgcaaacaaaacaaaaaacagcatagaagaaagagcaaaataaagaagaagaagaggaggaagagagggaaagagaggaagggaaaaaaaacaccaacccgggcagaggaggaggtgcggcggcggcggcggcggcggcagcggcggcagcggcgcggcggcggctcggaccccctcccccggctccccccatcagtgcagctctccgggcgatgccagaatagatgccggggcaatgtcccgccgcaaacagggcaacccgcagcacttgtcccagagggagctcatcaccccagaggctgaccatgtggaggccgccatcctcgaagaagacgagggtctggagatagaggagccaagtggcctggggctgatggtgggtggccccgaccctgacctgctcacctgtggccagtgtcaaatgaacttccccttgggggacatcctggtttttatagagcacaaaaggaagcagtgtggcggcagcttgggtgcctgctatgacaaggccctggacaaggacagcccgccaccctcctcacgctccgagctcaggaaagtgtccgagccggtggagatcgggatccaagtcacccccgacgaagatgaccacctgctctcacccacgaaaggcatctgtcccaagcaggagaacattgcagggccgtgcaggcctgcccagctgccagcggtggcccccatagctgcctcctcccaccctcactcatccgtgatcacttcacctctgcgtgccctgggcgctctcccgccctgcctccccctgccgtgctgcagcgcgcgcccggtctcgggtgacgggactcagggtgagggtcagacggaggctccctttggatgccagtgtcagttgtcaggtaaagatgagccttccagctacatttgcacaacatgcaagcagcccttcaacagcgcgtggttcctgctgcagcacgcgcagaacacgcacggcttccgcatctacctggagcccgggccggccagcagctcgctcacgccgcggctcaccatcccgccgccgctcgggccggaggccgtggcgcagtccccgctcatgaatttcctgggcgacagcaaccccttcaacctgctgcgcatgacgggccccatcctgcgggaccacccgggcttcggcgagggccgcctgccgggcacgccgcctctcttcagtcccccgccgcgccaccacctggacccgcaccgcctcagtgccgaggagatggggctcgtcgcccagcaccccagtgccttcgaccgagtcatgcgcctgaaccccatggccatcgactcgcccgccatggacttctcgcggcggctccgcgagctggcgggcaacagctccacgccgccgcccgtgtccccgggccgcggcaaccctatgcaccggctcctgaaccccttccagcccagccccaagtccccgttcctgagcacgccgccgctgccgcccatgccccctggcggcacgccgcccccgcagccgccagccaagagcaagtcgtgcgagttctgcggcaagaccttcaagttccagagcaatctcatcgtgcaccggcgcagtcacacgggcgagaagccctacaagtgccagctgtgcgaccacgcgtgctcgcaggccagcaagctcaagcgccacatgaagacgcacatgcacaaggccggctcgctggccggccgctccgacgacgggctctcggccgccagctcccccgagcccggcaccagcgagctggcgggcgagggcctcaaggcggccgacggtgacttccgccaccacgagagcgacccgtcgctgggccacgagccggaggaggaggacgaggaggaggaggaggaggaggaggagctgctactggagaacgagagccggcccgagtcgagcttcagcatggactcggagctgagccgcaaccgcgagaacggcggtggtggggtgcccggggtcccgggcgcggggggcggcgcggccaaggcgctggctgacgagaaggcgctggtgctgggcaaggtcatggagaacgtgggcctaggcgcactgccgcagtacggcgagctcctggccgacaagcagaagcgcggcgccttcctgaagcgtgcggcgggcggcggggacgcgggcgacgacgacgacgcgggcggctgcggggacgcgggcgcgggcggcgcggtcaacgggcgcgggggcggcttcgcgccaggcaccgagcccttccccgggctcttcccgcgcaagcccgcgccgctgcccagccccgggctcaacagcgccgccaagcgcatcaaggtggagaaggacctggagctgccgcccgccgcgctcatcccgtccgagaacgtgtactcgcagtggctggtgggctacgcggcgtcgcggcacttcatgaaggaccccttcctgggcttcacggacgcacgacagtcgcccttcgccacgtcgtccgagcactcgtccgagaacggcagcctgcgcttctccacgccgcccggggacctgctggacggcggcctctcgggccgcagcggcacggccagcggaggcagcaccccgcacctgggcggcccgggccccgggcggcccagctccaaggagggccgccgcagcgacacgtgcgagtactgcggcaaggtgttcaagaactgcagcaacttgacggtgcaccggcggagccacaccggcgagcggccttacaagtgcgagctgtgcaactacgcgtgcgcgcagagcagcaagctcacgcgccacatgaagacgcacgggcagatcggcaaggaggtgtaccgctgcgacatctgccagatgcccttcagcgtctacagcaccctggagaaacacatgaaaaagtggcacggcgagcacttgctgactaacgacgtcaaaatcgagcaggccgagaggagctaagcgcgcgggccccggcgccccgcacctgtacagtggaaccgttgccaaccgagagaatgctgacctgacttgcctccgtgtcaccgccaccccgcaccccgcgtgtccccggggcccaggggaggcggcactccaacctaacctgtgtctgcgaagtcctatggaaacccgagggttgattaaggcagtacaaattgtggagccttttaactgtgcaataatttctgtatttattgggttttgtaatttttttggcatgtgcaggtactttttattattattttttctgtttgaattcctttaagagattttgttgggtatccatcccttctttgttttttttttaacccggtagtagcctgagcaatgactcgcaagcaatgttagaggggaagcatatcttttaaattataatttggggggaggggtggtgctgcttttttgaaatttaagctaagcatgtgtaatttcttgtgaagaagccaacactcaaatgacttttaaagttgtttactttttcattccttccttttttttgtcctgaaataaaaagtggcatgcagttttttttttaattattttttaattttttttttggtttttgtttttggggtggggggtgtggatgtacagcggataacaatctttcaagtcgtagcactttgtttcagaactggaatggagatgtagcactcatgtcgtcccgagtcaagcggccttttctgtgttgatttcggctttcatattacataagggaaaccttgagtggtggtgctgggggaggcaccccacagactcagcgccgccagagatagggtttttggagggctcctctgggaaatggcccgacagcattctgaggttgtgcatgaccagcagatactatcctgttggtgtgccctggggtgccatggctgctattcgctgtagattaggctacataaaatgggctgagggtacctttttggggagatggggtggcctgcagtgacacagaaaggaagaaactagcggtgttcttttaggcgttttctggcttgacggcttctctctttttttaaatcacccccaccacataaatctcaaatcctatgttgctacaaggggtcatccatcatttcccaagcagacgaatgccctaattaattgaagttagtgttctctcatttaatgcacactgatgatattgtagggatgggtggggtggggatcttgcaaatttctattctcttttactgaaaaagcaggggatgagttccatcagaaggtgcccagcgctacttcccaggtttttattttttttttcctatctcattaggttggaaggtactaaatattgaactgttaagattagacatttgaattctgttgacccgcactttaaagcttttgtttgcatttaaattaaatggcttctaaacaagaaattgcagcatattcttctctttggcccagaggtgggttaaactgtaagggacagctgagattgagtgtcagtattgctaagcgtggcattcacaatactggcactataaagaacaaaataaaataataatttataggacagtttttctactgccattcaatttgatgtgagtgccttgaaaactgatcttcctatttgagtctcttgagacaaatgcaaaactttttttttgaaatgaaaagactttttaaaaaagtaaaacaagaaaagtacattctttagaaactaacaaagccacatttactttaagtaaaaaaaaaaaaaattctggttgaagatagaggatatgaaatgccataagacccaatcaaatgaagaaataaacccagcacaaccttggacatccattagctgaattatcctcagccccttttgtttttgggacaacgctgcttagatatggagtggaggtgatttactgctgaattaaaactcaagtgacacaagttacaagttgatatcgttgaatgaaaagcaaaacaaaaacaattcaggaacaacggctaattttttctaaagttaaatttagtgcactctgtcttaaaaatacgtttacagtattgggtacatacaagggtaaaaaaaaaattgtgtgtatgtgtgttggagcgatctttttttttcaaagtttgcttaataggttatacaaaaatgccacagtggccgcgtgtatattgttttcttttggtgacggggttttagtatatattatatatattaaaatttcttgattactgtaaaagtggaccagtatttgtaataatcgagaatgcctgggcattttacaaaacaagaaaaaaaatacccttttcttttccttgaaaatgttgcagtaaaatttaaatggtgggtctataaatttgttcttgttacagtaactgtaaagtcggagttttagtaaatttttttctgccttgggtgttgaatttttatttcaaaaaaaatgtatagaaacttgtatttggggattcaaaggggattgctacaccatgtagaaaaagtatgtagaaaaaaagtgcttaatattgttattgctttgcagaaaaaaaaaaaatcacatttctgacctgtacttatttttctcttcccgcctccctctggaatggatatattggttggttcatatgatgtaggcacttgctgtatttttactggagctcgtaattttttaactgtaagcttgtccttttaaagggatttaatgtacctttttgttagtgaatttggaaataaaaagaaaaaaaaaacaaaaacaaacaggctgccataatatatttttttaatttggcaggataaaatattgcaaaaaaaacacatttgtatgttaagtcctattgtacaggagaaaaagggttgtttgacaacctttgagaaaaagaaacaaaaggaagtagttaaatgctttggttcacaaatcatttagttgtatatattttttgtcggaattggcctacacagagaaccgttcgtgttgggcttctctctgaacgccccgaaccttgcatcaaggctccttggtgtggccacagcagaccagatgggaaattatttgtgttgagtggaaaaaaatcagtttttgtaaagatgtcagtaacattccacatcgtcctccctttctctaagaggccatctctaagatgtcagatgtagaggagagagagcgagagaacatcttccttctctaccatcactcctgtggcggtcaccaccaccacctctcccgcccttaccagcagaaagcaatgcaaactgagctgctttagtccttgagaaattgtgaaacaaacacaaatatcataaaaggagctggtgattcagctgggtccaggtgaagtgacctgctgttgagaccggtacaaattggatttcaggaaggagactccatcacagccaggacctttcgtgccatggagagtgttggcctcttgtctttcttccctgctttgctgctttgctctctgaaacctacattccgtcagtttccgaatgcgagggcctgggatgaatttggtgcctttccatatctcgttctctctccttcccctgcgtttcctctccatccttcatcctccattggtcctttttttttctttcattttttatttaatttcttttcttcctgtctgttcctcccctaatcctctattttatttttattttttgtaaagccaagtagctttaagataaagtggtggtcttttggatgagggaataatgcatttttaaataaaataccaatatcaggaagccattttttatttcaggaaatgtaagaaaccattatttcaggttatgaaagtataaccaagcatccttttgggcaattccttaccaaatgcagaagcttttctgttcgatgcactctttcctccttgccacttacctttgcaaagttaaaaaaaaggggggagggaatgggagagaaagctgagatttcagtttcctactgcagtttcctacctgcagatccaggggctgctgttgcctttggatgccccactgaggtcctagagtgcctccagggtggtcttcctgtagtcataacagctagccagtgctcaccagcttaccagattgccaggactaagccatcccaaagcacaagcattgtgtgtctctgtgactgcagagaagagagaattttgcttctgttttgtgtttaaaaaaccaacacggaagcagatgatcccgagagagaggcctctagcatgggtgacccagccgacctcaggccggtttccgcactgccacaactttgttcaaagttgcccccaattggaacctgccacttggcattagagggtctttcatggggagagaaggagactgaattactctaagcaaaatgtgaaaagtaaggaaatcagcctttcatcccggtcctaagtaaccgtcagccgaaggtctcgtggaacacaggcaaacccgtgattttggtgctccttgtaactcagccctgcaaagcaaagtcccattgatttaagttgtttgcatttgtactggcaaggcaaaatatttttattaccttttctattacttattgtatgagcttttgttgtttacttggaggttttgtcttttactacaagtttggaactatttattattgcttggtatttgtgctctgtttaagaaacaggcacttttttttattatggataaaatgttgagatgacaggaggtcatttcaatatggcttagtaaaatatttattgttcctttattctctgtacaagattttgggcctctttttttccttaatgtcacaatgttgagttcagcatgtgtctgccatttcatttgtacgcttgttcaaaaccaagtttgttctggtttcaagttataaaaataaattggacatttaacttgatctccaaa

SEQ ID NO: 28 - DNA, 1851 bp, Homo sapiens
gtgcaggtgaggcacgtttagcctgagccggccacggactccacttcccctgctcttcccccagtggcaaatccgcgccacctcgcaaacccccaactcaggcacttgggccccttttgggccccctctcgctcctccctttaggcacctccctgggcccgcccacggtctccccccagtttgggactgcgtcataagtatcccagacctcggcttgcagtagtgttagactgaagataaagtaagtgctgtttgggctaacaggatctcctcttgcagtctgcagcccaggacgctgattccagcagcgccttaccgcgcagcccgaagattcactatggtgaaaatcgccttcaatacccctaccgccgtgcaaaaggaggaggcgcggcaagacgtggaggccctcctgagccgcacggtcagaactcagatactgaccggcaaggagctccgagttgccacccaggaaaaagagggctcctctgggagatgtatgcttactctcttaggcctttcattcatcttggcaggacttattgttggtggagcctgcatttacaagtacttcatgcccaagagcaccatttaccgtggagagatgtgcttttttgattctgaggatcctgcaaattcccttcgtggaggagagcctaacttcctgcctgtgactgaggaggctgacattcgtgaggatgacaacattgcaatcattgatgtgcctgtccccagtttctctgatagtgaccctgcagcaattattcatgactttgaaaagggaatgactgcttacctggacttgttgctggggaactgctatctgatgcccctcaatacttctattgttatgcctccaaaaaatctggtagagctctttggcaaactggcgagtggcagatatctgcctcaaacttatgtggttcgagaagacctagttgctgtggaggaaattcgtgatgttagtaaccttggcatctttatttaccaactttgcaataacagaaagtccttccgccttcgtcgcagagacctcttgctgggtttcaacaaacgtgccattgataaatgctggaagattagacacttccccaacgaatttattgttgagaccaagatctgtcaagagtaagaggcaacagatagagtgtccttggtaataagaagtcagagatttacaatatgactttaacattaaggtttatgggatactcaagatatttactcatgcatttactctattgcttatgctttaaaaaaaggaaaaaaaaaaactactaaccactgcaagctcttgtcaaattttagtttaattggcattgcttgttttttgaaactgaaattacatgagtttcattttttctttgaatttatagggtttagatttctgaaagcagcatgaatatatcacctaacatcctgacaataaattccatccgttgttttttttgtttgtttgttttttcttttcctttaagtaagctctttattcatcttatggtggagcaattttaaaatttgaaatattttaaattgtttttgaactttttgtgtaaaatatatcagatctcaacattgttggtttcttttgtttttcattttgtacaactttcttgaatttagaaattacatctttgcagttctgttaggtgctctgtaattaacctgacttatatgtgaacaattttcatgagacagtcatttttaactaatgcagtgattctttctcactactatctgtattgtggaatgcacaaaattgtgtaggtgctgaatgctgtaaggagtttaggttgtatgaattctacaaccctataataaattttactctatacaaaaaaaaaaaaaaaaaaa

SEQ ID NO: 29 - DNA, 2751 bp, Homo sapiens
agtttatgacagaagggcaaaaacattgactgcctcaaggtctcaagcaccagtcttcaccgcggaaagcatgttgtggctgttccaatcgctcctgtttgtcttctgctttggcccagggaatgtagtttcacaaagcagcttaaccccattgatggtgaacgggattctgggggagtcagtaactcttcccctggagtttcctgcaggagagaaggtcaacttcatcacttggcttttcaatgaaacatctcttgccttcatagtaccccatgaaaccaaaagtccagaaatccacgtgactaatccgaaacagggaaagcgactgaacttcacccagtcctactccctgcaactcagcaacctgaagatggaagacacaggctcttacagagcccagatatccacaaagacctctgcaaagctgtccagttacactctgaggatattaagacaactgaggaacatacaagttaccaatcacagtcagctatttcagaatatgacctgtgagctccatctgacttgctctgtggaggatgcagatgacaatgtctcattcagatgggaggccttgggaaacacactttcaagtcagccaaacctcactgtctcctgggaccccaggatttccagtgaacaggactacacctgcatagcagagaatgctgtcagtaatttatccttctctgtctctgcccagaagctttgcgaagatgttaaaattcaatatacagataccaaaatgattctgtttatggtttctgggatatgcatagtcttcggtttcatcatactgctgttacttgttttgaggaaaagaagagattccctatctttgtctactcagcgaacacagggccccgagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaagttgctgaaaggcctcagaggaattcgggaatgacacgtcttctgatcccatgagacagaacaaagaacaggaagcttggttcctgttgttcctggcaacagaatttgaatatctaggataggatgatcacctccagtccttcggacttaaacctgcctacctgagtcaaacacctaaggataacatcatttccagcatgtggttcaaataatattttccaatccacttcaggccaaaacatgctaaagataacacaccagcacattgactctctctttgataactaagcaaatggaattatggttgacagagagtttatgatccagaagacaaccacttctctccttttagaaagcagcaggattgacttattgagaaataatgcagtgtgttggttacatgtgtagtctctggagttggatgggcccatcctgatacaagttgagcatcccttgtctgaaatgcttgggattagaaatgtttcagatttcaattttttttcagattttggaatatttgcattatatttagcggttgagtatccaaatccaaaaatccaaaattcaaaatgctccaataagcatttcccttgagtttcattgatgtcgatgcagtgctcaaaatctcagattttggagcattttggatattggatttttggatttgggatgctcaacttgtacaatgtttattagacacatctcctgggacatactgcctaaccttttggagccttagtctcccagactgaaaaaggaagaggatggtattacatcagctccattgtttgagccaagaatctaagtcatccctgactccagtgtctttgtcaccaggccctttggactctacctcagaaatatttcttggaccttccacttctcctccaactccttgaccaccatcctgtatccaaccatcaccacctctaacctgaatcctaccttaagatcagaacagttgtcctcacttttgttcttgtccctctccaacccactctccacaagatggccagagtaatgtttttaatataaattggatccttcagtttcctgcttaaaaccctgcaggtttcccaatgcactcagaaagaaatccagtttccatggccctggatggtctggcccacctccagcctcagctagcattacccttctgacactctctatgtagcctccctgatcttctttcagctcctctattaaaggaaaagttctttatgttaattatttacatcttcctgcaggcccttcctctgcctgctggggtcctcctattctttaggtttaattttaaatatgtcacctcctaagagaaaccttcccagaccactctttctaaaatgaatcttctaggctgggcatggtggctcacacctgtaatccctgtactttgggaggccaaggggggagatcacttgaggtcaggagttcaagaccagcctggccaacttggtgaaaccccgtctttactaaaaatacaaaaaaattagccaggcgtggtggtgcacccctaaaatcccagctacttgagagactgaggcaggagaatcgcttgaacccaggaggtggaggttccagtgagccaaaatcatgccaatgtattccagtctgggtgacagagtgagactctgtctcaaaaaataaataaataaaataaaatgaaatagatcttataaaaaaaa

SEQ ID NO: 30 - DNA, 4055 bp, Homo sapiens
gtcacagaagactacttgggttcatggtctctaatatttcaaacaggagctccctttagcgagtccttcttttcctgactgcagctcttttcattttgccatccttttccagctccatgatggttctgcaggtttctgcggccccccggacagtggctctgacggcgttactgatggtgctgctcacatctgtggtccagggcagggccactccagagaattaccttttccagggacggcaggaatgctacgcgtttaatgggacacagcgcttcctggagagatacatctacaaccgggaggagttcgcgcgcttcgacagcgacgtgggggagttccgggcggtgacggagctggggcggcctgctgcggagtactggaacagccagaaggacatcctggaggagaagcgggcagtgccggacaggatgtgcagacacaactacgagctgggcgggcccatgaccctgcagcgccgagtccagcctagggtgaatgtttccccctccaagaaggggcccttgcagcaccacaacctgcttgtctgccacgtgacggatttctacccaggcagcattcaagtccgatggttcctgaatggacaggaggaaacagctggggtcgtgtccaccaacctgatccgtaatggagactggaccttccagatcctggtgatgctggaaatgaccccccagcagggagatgtctacacctgccaagtggagcacaccagcctggatagtcctgtcaccgtggagtggaaggcacagtctgattctgcccggagtaagacattgacgggagctgggggcttcgtgctggggctcatcatctgtggagtgggcatcttcatgcacaggaggagcaagaaagttcaacgaggatctgcataaacagggttcctgagctcactgaaaagactattgtgccttaggaaaagcatttgctgtgtttcgttagcatctggctccaggacagaccttcaacttccaaattggatactgctgccaagaagttgctctgaagtcagtttctatcattctgctctttgattcaaagcactgtttctctcactgggcctccaaccatgttcccttcttcttagcaccacaaataatcaaaacccaacatgactgtttgttttcctttaaaaatatgcaccaaatcatctctcatcacttttctctgagggttttagtagacagtaggagttaataaagaagttcattttggtttaaacataggaaagaagagaaccatgaaaatggggatatgttaactattgtataatggggcctgttacacatgacactcttctgaattgactgtatttcagtgagctgcccccaaatcaagtttagtgccctcatccatttatgtctcagaccactattcttaactattcaatggtgagcagactgcaaatctgcctgataggacccatattcccacagcactaattcaacatataccttactgagagcatgttttatcattaccattaagaagttaaatgaacatcagaatttaaaatcataaatataatctaatacactttaaccattttctttgtgtgccatcacaaatactccttaaccaaatacggcttggacttttgaatgcatccaatagacgtcatttgtcgtctaagtctgcattcatccaccagcctaggcctcctgtcttaattttcatacagacagaaatgactccccactggggaaagagcaaagcaatacatgtagcactctttttcaaacactggtctttttttttttcttaacaatccaacattgttatgtgttttgcgtctcatattgacaccttttggtcaaggtagaggacatgtttgttgtaagctttctttttcgtgtagaggatggattcttcactcctgatacacacaatcagtgcacagcagctctcttatacatccagttgatgccttcagtctccctggcttcttacaagcatcttctgggccttgtgtgtccctgggcacctgtccctggtcaattcccgaaagctactgtgctcctcttgcccatctccccttgcaaataatatcttccatcgggggaccggcttcctccaatttcaggagaggtggggctgaaggcacagacttgggcgtcactggcacagatataagtaaatacagctggagtctgcagagaggctggactgagtcagggagtcaggaaagagaagccacacacaaggacaaccaatcatgtttctcataatcttcttaacctagggaataggacacaatcattttttctttttaaaacatctttatccctgatcagcctcatttcctcaaaaactataaaggaaaatgctgctgacttgtttttgcgtagtaatttcagctgtcacataataagctaaggaagacagtatatagtaaataaggaccctttatctgtcttattttcccttttggcttcacaggaaacttgtgagaaacctatgcagcataaaattaatatgatttcaatccagggattcaacgatggaaggaggtcatgagaatagcagaaagtcttcaaatcgagatcattatgaaatcctcagacccagagcacataaatcctaccctcagagtcactgagcagttaacattacaaattacaaaccatatccagtcagagtcattctctttcctgcttgtctcctgtactcatgttacaggttagggcagtaccccgagtggagtgaacaatctctggactaacacttgtcaggatcagaagctgaggtatctgcacccacattacaggaacaggatatgtgctcctagggaactgagggtgtcaggagatgaggaatgtccctggagtcacagaaagaaggtatcagatgtgtctcactctgacatatgcaggtgtttatgaaactctgggatttctaaggaaggatgcagtgcagagacaggtcccagaggagacaagagctgagagaccatccaaactgggaccaccttgtcactagacttcaaattttcaatattgatagagtgttttctaagagtcaggccctttgctgagtgctatgtgcagcaggatcaaaggcagccaggaggtagaggagtcttgaggtacatcagtcattggagttgaagagcagagattcaaaggaaagttggaactggagctttaaaggagatgtgaagtgggtgactcaacctctgactcagaaaaattgatacctgcagaagaaaaaacccggcgggcttaggactcccagctgagtgttgtatcctccatccctttccacctggtcccttcattttctacccctcacagttccctaacgagaaggtggtccacccaacagacaacactgcctcagatggttatcaaggggtaccctaagaagaaatcatctcaccctctctttgtccccatttgtcaagtagcagtgaggccgagccaggggatggtgaaagtggaaggaggtgggagttgggcatcgggtgtgaagatgctcttgaaaggggttttaataaccacttgctaccaggccagtgaacacttaccatagttgatgccttttgagcatgttgcattgtaaactgtccctgaaattactgtgcacttggcttatgggatgaaacatcctcctagttcttttgtctctcagcttctctgaagtctcattgagcaccttctcttcaatttcttttacacagtaagaataggatcagctgtgctaaactaacaaatacccagatatccaggtttggctcatgttacacgtccaaagtaagtcatgcaggaagctctgctcatcatcgtactcaggaagtcaggctgacagtctttctcctgcacatctgctcccagaacctccccagcagaatgaagggaacctaagaatttattcactggcttttaatgatccctcctagaaagaacacacttctcgcatttcattttccaatgtaaatcatatggctgcaactaacttcaaataagtgggaatacttgaaggtggaaaacatttaagaagtacacactaaataaataataaaatacttctacaagaga

SEQ ID NO: 31 - DNA, 1065 bp, Homo sapiens
atcactcagtgcccctgagctcattcttttcagtaaattctctctctgcgtggtgagaaaacaggcctggagaggctctgcgacccgcttaggaccacagaactcgagaattaccttttccagggacggcaggaatgctacgcgtttaatgggacacagcgcttcctggagagatacatctacaaccgggaggagctcgtgcgcttcgacagcgacgtgggggagttccgggcggtgacggagctggggcggcctgaggcggagtactggaacagccagaaggacatcctggaggaggagcgggcagtgccggacaggatgtgcagacacaactacgagctgggcgggcccatgaccctgcagcgccgagtccagcctagggtgaatgtttccccctccaagaaggggcccttgcagcaccacaacctgcttgtctgccacgtgacggatttctacccaggcagcattcaagtccgatggttcctgaatggacaggaggaaacagctggggtcgtgtccaccaacctgatccgtaatggagactggaccttccagatcctggtgatgctggaaatgaccccccagcagggagatgtctacacctgccaagtggagcacaccagcctggatagtcctgtcaccgtggagtggaaggcacagtctgattctgcccggagtaagacattgacgggagctgggggcttcgtgctggggctcatcatctgtggagtgggcatcttcatgcacaggaggagcaagaaagttcaacgaggatctgcataaacagggttcctgagctcactgaaaagactattgtgccttaggaaaagcatttgctgtgtttcgttagcatctggctccaggacagaccttcaacttccaaattggatactgctgccaagaagttgctctgaagtcagtttctatcattctgctctttgattcaaagcactgtttctctcactgggcctccaaccatgttcccttcttcttagcaccacaaataatcaaaacccaacatgactgtttgttttcctttaaaaatatgcaccaaatcatctctca

SEQ ID NO: 32 - DNA, 1633 bp, Homo sapiens
gacgaaactggagagatagggttttaacaagatgcaaggacaatctgaggactgagagccatttcaacgtgagcccccagtctgagaacaagaaagaagaacttctgtctcgagggtctcactgtcaaccaggccagagtgcagtgaagatcatacctcactacatccgtgaactcccgggctcctcccacctaagtctcttgagtagctgggacttcaggagactgaagccaaggataccagcagagccaacatttgcttcaagttcctgggcctgctgacagcgtgcaggatgctgttggaacccggcagaggctgctgtgccctggccatcctgctggcaattgtggacatccagtctggtggatgcattaacatcaccagctcagcttcccaggaaggaacgcgactaaacttaatctgtactgtatggcataagaaagaagaggctgaggggtttgtagtgtttttgtgcaaggacaggtctggagactgttctcctgagaccagtttaaaacagctgagacttaaaagggatcctgggatagatggtgttggtgaaatatcatctcagttgatgttcaccataagccaagtcacaccgttgcacagtgggacctaccagtgttgtgccagaagccagaagtcaggtatccgccttcagggccattttttctccattctattcacagagacagggaactacacagtgacgggattgaaacaaagacaacaccttgagttcagccataatgaaggcactctcagttcaggcttcctacaagaaaaggtctgggtaatgctggtcaccagccttgtggcccttcaagctttgtaagccttgtgccaaaagaaacttttaaaacagctacagcaagatgagtctgactatggcttagtatctttctcattacaataggcacagagaagaatgcaacagggcacaggggaagagatgctaaatataccaagaatctgtggaaatataagctggggcaaatcagtgtaatccttgactttgctcctcaccatcagggcaaacttgccttcttccctcctaagctccagtaaataaacagaacagctttcaccaaagtgggtagtatagtcctcaaatatcggataaatatatgcgtttttgtaccccagaaaaacttttcctccctcttcatcaacatagtaaaataagtcaaacaaaatgagaacaccaaattttgggggaataaatttttatttaacactgcaaaggaaagagagagaaaacaagcaaagataggtaggacagaaaggaagacagccagatccagtgattgacttggcatgaaaatgagaaaatgcagacagacctcaacattcaacaacatccatacagcactgctggaggaagaggaagatttgtgcagaccaagagcaccacagactacaactgcccagcttcatctaaatacttgttaacctctttggtcatttctctttttaaataaatgcccatagcagtatttggagtcttttcttttctcctaaatccacaaactctcttctttctctttggacagatgacctcttgtcatagttaagcagagagtgggcaggatattcctgataggaggaactacatgaataaaggggtaag

SEQ ID NO: 33 - DNA, 1266 bp, Homo sapiens
atttcatgttatacttaataaaacaaaacatacctgtatacacacacattcactcacattgaagatgcaagatgaagaaagatacatgacattgaatgtacagtcaaagaaaaggagttctgcccaaacatctcaacttacatttaaagattattcagtgacgttgcactggtataaaatcttactgggaatatctggaaccgtgaatggtattctcactttgactttgatctccttgatcctgttggtttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattagagtttgacaaaattcacagtgaaataatcaatgatcactatttttggcctattagtttctaatattaatctccaggtgtaagattttaaagtgcaattaaatgccaaaatctcttctcccttctccctccatcatcgacactggtctagcctcagagtaacccctgttaacaaactaaaatgtacacttcaaaatttttacgtgatagtataaaccaatgtgacttcatgtgatcatatccaggatttttattcgtcgcttattttatgccaaatgtgatcaaattatgcctgtttttctgtatcttgcgttttaaattcttaataaggtcctaaacaaaatttcttatatttctaatggttgaattataatgtgggtttatacattttttacccttttgtcaaagagaattaactttgtttccaggcttttgctactcttcactcagctacaataaacatcctgaatgttttcttaaaaaaaaaaaaaaaaaaaaaaaaaaaaaa

SEQ ID NO: 34 - DNA, 1595 bp, Homo sapiens
agaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaaaaaaaaaaaaaaaaaaaaaaaaaaaaaaaaaaaaaaaaaaaaaaaaa

SEQ ID NO: 35 - DNA, 543 bp, Homo sapiens
gaccttgagggagttaatgtgtaatattctaggatataagcttgaccacgagttgagaccctgagcacaggcctccaggagccgctgggagctgccgccaggagctgtcaccatgacgggggaacttgaggttaagaacatggacatgaagccggggtcaaccctgaagatcacaggcagcatcgccgatggcactgatggctttgtaattaatctgggccaggggacagacaagctgaacctgcatttcaaccctcgcttcagcgaatccaccattgtctgcaactcattggacggcagcaactgggggcaagaacaacgggaagatcacctgtgcttcagcccagggtcagaggtcaagttcacagtgacctttgagagtgacaaattcaaggtgaagctgccagatgggcacgagctgacttttcccaacaggctgggtcacagccacctgagctacctgagcgtaaggggcgggttcaacatgtcctctttcaagttaaaagaataaaagacttccagccgagaaaaaaaaaaaaaaaa

SEQ ID NO: 36 - DNA, 777 bp, Homo sapiens
cgggctcagagcgcacccagccggcgccgcgcagcactgggaccctgctcgccctgcagcccagccagcctgctccgcatccccctgctggtctgcccgccgacctgcgcgccctcgctgccgcccgtgtgcgcccctcgaccccagcggcaccatgcatctctcccagctgctggcctgcgccctgctgctcacgctgctctccctccggccctccgaagccaagcccggggcgccgccgaaggtcccgcgaaccccgccggcagaggagctggccgagccgcaggctgcgggcggcggtcagaagaagggcgacaaggctcccgggggcgggggcgccaatctcaagggcgaccggtcgcgactgctccgggacctgcgcgtggacaccaagtcgcgggcagcgtgggctcgccttctgcaagagcaccccaacgcgcgcaaatacaaaggagccaacaagaagggcttgtccaagggctgcttcggcctcaagctggaccgaatcggctccatgagcggcctgggatgttagtgcggcgccccctggcggcggatcgggaactggctccgttgtgctgaggtcatctttggtcatcagcctccagcatctggaaacacctccaacgcaatgtggcttttacatttctttctttctttctttttttttcctggtactgggaatacacaacaccagctgttttattattatttggggagggggttgtgattttattatttgtttttttaaaatgaaaaataaaaagttatatatta

SEQ ID NO: 37 - DNA, 3624 bp, Homo sapiens
aatgcgcctgcagctcgcgctcccgcgccgatcccgagagcgtccgggccgccgtgcgcgagcgagggagggcgcgcgcgcggggggggcgcgcttgtgagtgcgggccgcgctctcggcggcgcgcatgtgcgtgtgtgctggctgccgggctgccccgagccggcggggagccggtccgctccaggtggcgggcggctggagcgaggtgaggctgcgggtggccagggcacgggcgcgggtcccgcggtgcgggctggctgcaggctgccttctgggcacggcgcgcccccgcccggccccgccgggccctgggagctgcgctccgggcggcgctggcaaagtttgctttgaactcgctgcccacagtcgggtccgcgcgctgcgattggcttcccctaccactctgacccggggcccggcttcccgggacgcgaggactgggcgcaggctgcaagctggtggggttggggaggaacgagagcccggcagccgactgtgccgagggacccggggacacctccttcgcccggccggcacccggtcagcacgtccccccttccctcccgcagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaactgaccaaaaagcctgacagttctgtcttacgaagacacaagtttattttttaacctccctctcccctttagtaatttgcacattttggttatggtgggacagtctggacagtagatcccagaatgcattgcagccggtgcacacacaataaaggcttgcattcttggaaaccttgaaacccagctctccctcttccctgactcatgggagtgctgtatgttctctggcgcctttggcttcccagcaggcagctgactgaggagccttggggtctgcctagctcactagctctgaagaaaaggctgacagatgctatgcaacaggtggtggatgttgtcaggggctccagcctgcatgaaatctcacactctgcatgagctttaggctaggaaaggatgctcccaactggtgtctctggggtgatgcaaggacagctgggcctggatgctctccctgaggctcctttttccagaagacacacgagctgtcttgggtgaagacaagcttgcagacttgatcaacattgaccattacctcactgtcagacactttacagtagccaaggagttggaaacctttatatattatgatgttagctgacccccttcctcccactcccaatgctgcgaccctgggaacacttaaaaagcttggcctctagattctttgtctcagagccctctgggctctctcctctgagggagggacctttctttcctcacaagggacttttttgttccattatgccttgttatgcaatgggctctacagcaccctttcccacaggtcagaaatatttccccaagacacagggaaatcggtcctagcctggggcctggggatagcttggagtcctggcccatgaacttgatccctgcccaggtgttttccgaggggcacttgaggcccagtcttttctcaaggcaggtgtaagacacctcagagggagaactgtactgctgcctctttcccacctgcctcatctcaatccttgagcggcaagtttgaagttcttctggaaccatgcaaatctgtcctcctcatgcaattccaaggagcttgctggctctgcagccacccttgggccccttccagcctgccatgaatcagatatctttcccagaatctgggcgtttctgaagttttggggagagctgttgggactcatccagtgctccagaaggtggacttgcttctggtgggttttaaaggagcctccaggagatatgcttagccaaccatgatggattttaccccagctggactcggcagctccaagtggaatccacgtgcagcttctagtctgggaaagtcacccaacctagcagttgtcatgtgggtaacctcaggcacctctaagcctgtcctggaagaaggaccagcagcccctccagaactctgcccaggacagcaggtgcctgctggctctgggtttggaagttggggtgggtagggggtggtaagtactatatatggctctggaaaaccagctgctacttccaaatctattgtccataatggtttctttctgaggttgcttcttggcctcagaggaccccaggggatgtttggaaatagcctctctacccttctggagcatggtttacaaaagccagctgacttctggaattgtctatggaggacagtttgggtgtaggttactgatgtctcaactgaatagcttgtgttttataagctgctgttggctattatgctgggggagtcttttttttttatattgtatttttgtatgccttttgcaaagtggtgttaactgtttttgtacaaggaaaaaaactcttggggcaatttcctgttgcaagggtctgatttattttgaaaggcaagttcacctgaaattttgtatttagttgtgattactgattgcctgattttaaaatgttgccttctgggacatcttctaataaaagatttctcaaacatgtc

SEQ ID NO: 38 - DNA, 2050 bp, Homo sapiens
aatgcgcctgcagctcgcgctcccgcgccgatcccgagagcgtccgggccgccgtgcgcgagcgagggagggcgcgcgcgcggggggggcgcgcttgtgagtgcgggccgcgctctcggcggcgcgcatgtgcgtgtgtgctggctgccgggctgccccgagccggcggggagccggtccgctccaggtggcgggcggctggagcgagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcgggtaaggacctccccgagccatccaagagggggccaccccatgggtggccaaagctctgcccctgcctgaggtcaggcattggctcttctcaagctcttgggccatctccgcctctctttggctgaagctgcccgtgtagtccccaaccgtgtctgtctggcacgtgggtgtgttggtaaacagtttggaaaagtggcgtgggagccagcctccctttgatgattattggagccccaggggacaagggatttgaggtgagggttggcgcttagagaggacaatactggggttggactgtaagggattgaagggggtaccttaagagacactccaaacctgaagtttttttgctgctgcctctttccctaggaaactcacactcccctagggggagaagaagccgagagccttttgtgcaaagccaaaaccttcgtccttttaaaaacctaggtctccagttggctttactttaaaatgccaataataaatgccctcttctcgtgcctccccaccaccacttaccactcgtgcatccctgagacagggagggaagaatgaacactccccattaacagatggaaaaactgaggcttagagatagacaatcactacaagtcagctccagctttctgccatctagccagcccctcttccccaatgctccatcccaaccaggcacctcttccttgatgtttggggtctttgtggtagcttatcttagaagcactacaccttgccttgctgtttgtcctgagatggaaaagtgtccttcttgctccccctcaatagatctccagcgtcagctgctccctggcattcaacaaatattcactggcccctactttgtggcaatctgtgggctacatgctggggtcaaggcagtagaactccaggccctcctctcccatccttgatgcaagtgcaacctcgctgagggcagactggggcatcctgtgccactaaactacattgttcttattctggcatcttagacctccacacccgtgagaaatcctggagagggtatttttgtagagtgtagactgtggctagtgacaaataaattaggaccaagaaagctcactgtagcttttaggaataacttttacacgaccatttgatagggaactggggaatggggtatggaagttttcctacacttgagagaaaaaataggataacaaaaattaaaagtcttttttttcctggtccactgtgttaaggtcatttttaaccagcttgctttctacaccaagagtttatgtttgtttaatggctggaaagagaatcttgagatcaaaaaaccaataaagatgtatctctacaaaaaaaaaaaa

SEQ ID NO: 39 - DNA, 3444 bp, Homo sapiens
cgagcagaaatgaaaccgaaactgaattgtccgggaaattcgcggtgggggcggagagcgcagggagaagtaagcccagtgcaggatcctgaggcccgtgtttgcaggaccagggccggccttccgattccccattcattccagaagcaccgaaccacgctgtgcccggatcccaagtgcagcggcacccagcgtgggcctggggttgccggttgacccggtcctcagcctggtagcagaggccaggccagtgccacaaggcacctaagtccacctgggcctggagcaggacaggttgcaaaagaaaatatctcgggacccccaaactccttatgctaagggaaacatcgagcctgggaactgagccatcaacgctgccattctttttcccaaacagaaccctgttgtcagaggtacacccagagcaactccacaccgggtgcatgccacagcaactccatcttaaataggagctggtaaaacgaggctgatacctactgggctgcattcccagacggcatagcgaggaggtgctgaagagcgcaggtttggagaatgatcacctggattggaaccatagctctaccaatatggaacccagctccttaggcctcggtcttctcatggagaacatggtgtgataatcctactcctctgggagggtggctgttaagccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaagttaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 40 - DNA, 1237 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 41 - DNA, 2805 bp, Homo sapiens
ctccttgcacgggccggcccagcttccccgcccctggcgtccgctccctcccgctcgcagcttacttaacctggcccgggcggcggaggcgctctcacttccctggagccgcccgcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctgctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 42 - DNA, 1880 bp, Homo sapiens
gggtcggggccacaaggccgcgctaggcggacccaggacacagcccgcgcgcagcccacccgcccgccgcctgccagagctgctcggcccgcagccagggggacagcggctggtcggaggctcgcagtgctgtcggcgagaagcagtcgggtttggagcgcttgggtcgcgttggtgcgcggtggaacgcgcccagggaccccagttcccgcgagcagctccgcgccgcgcctgagagactaagctgaaactgctgctcagctcccaagatggtgccacccaaattgcatgtgcttttctgcctctgcggctgcctggctgtggtttatccttttgactggcaatacataaatcctgttgcccatatgaaatcatcagcatgggtcaacaaaatacaagtactgatggctgctgcaagctttggccaaactaaaatcccccggggaaatgggccttattccgttggttgtacagacttaatgtttgatcacactaataagggcaccttcttgcgtttatattatccatcccaagataatgatcgccttgacaccctttggatcccaaataaagaatatttttggggtcttagcaaatttcttggaacacactggcttatgggcaacattttgaggttactctttggttcaatgacaactcctgcaaactggaattcccctctgaggcctggtgaaaaatatccacttgttgttttttctcatggtcttggggcattcaggacactttattctgctattggcattgacctggcatctcatgggtttatagttgctgctgtagaacacagagatagatctgcatctgcaacttactatttcaaggaccaatctgctgcagaaataggggacaagtcttggctctaccttagaaccctgaaacaagaggaggagacacatatacgaaatgagcaggtacggcaaagagcaaaagaatgttcccaagctctcagtctgattcttgacattgatcatggaaagccagtgaagaatgcattagatttaaagtttgatatggaacaactgaaggactctattgatagggaaaaaatagcagtaattggacattcttttggtggagcaacggttattcagactcttagtgaagatcagagattcagatgtggtattgccctggatgcatggatgtttccactgggtgatgaagtatattccagaattcctcagcccctcttttttatcaactctgaatatttccaatatcctgctaatatcataaaaatgaaaaaatgctactcacctgataaagaaagaaagatgattacaatcaggggttcagtccaccagaattttgctgacttcacttttgcaactggcaaaataattggacacatgctcaaattaaagggagacatagattcaaatgtagctattgatcttagcaacaaagcttcattagcattcttacaaaagcatttaggacttcataaagattttgatcagtgggactgcttgattgaaggagatgatgagaatcttattccagggaccaacattaacacaaccaatcaacacatcatgttacagaactcttcaggaatagagaaatacaattaggattaaaataggttttttaaaagtcttgtttcaaaactgtctaaaattatgtgtgtgtgtgtgtgtgtgtgtgtgtgtgtgagagagagagagagagagagagagagagagagagagaattttaatgtattttcccaaaggactcatattttaaaatgtaggctatactgtaatcgtgattgaagcttggactaagaattttttccctttagatgtaaagaaagaatacagtatacaatattcaaaaaaaaaaaaaaaaaaaaaaaaaaaaaaaaaaaaaaaaaa

SEQ ID NO: 43 - DNA, 4502 bp, Homo sapiens
gcgccgtcccggccatgggcgcccgcggcggcctgcggagctggaggcgcggcgccggccgccaggcgcctttgtgagcggcgcggacgacaaaggcgcgggcccgggcagccgaggtgtgtagctgggacggtgctggtctgagctggaccttgtctgatggcttcctccaaccctcctccacagcctgccataggagatcagctggttccaggagtcccaggcccctcctctgaggcagaggacgacccaggagaggcgtttgagtttgatgacagtgatgatgaagaggacaccagcgcagccctgggcgtccccagccttgctcctgagagggacacagaccccccactgatccacttggactccatccctgtcactgacccagacccagcagctgctccacccggcacaggggtgccagcctgggtgagcaatggggatgcagcggacgcagccttctccggggcccggcactccagctggaagcggaagagttcccgtcgcattgaccggttcactttccccgccctggaagaggatgtgatttatgacgacgtcccctgcgagagcccagatgcgcatcagcccggggcagagaggaacctgctctacgaggatgcgcaccgggctggggcccctcggcaggcggaggacctaggctggagctccagtgagttcgagagctacagcgaggactcgggggaggaggccaagccggaggtcgaggtcgagcccgccaagcaccgagtgtccttccagcccaagctttctccagacctgactaggctaaaggagagatacgccaggactaagagagacatcttggctttgagagttggggggagagacatgcaggagctgaagcacaagtacgattgtaagatgacccagctcatgaaggccgccaagagcgggaccaaggatgggctggagaagacacggatggccgtgatgcgcaaagtctccttcctgcacaggaaggacgtcctcggtgactcggaggaggaggacatggggctcctggaggtcagcgtttcggacatcaagcccccagccccagagctgggccccatgccagagggcctgagccctcagcaggtggtccggaggcatatcctgggctccatcgtgcagagcgaaggcagctacgtggagtctctgaagcggatactccaggactaccgcaaccccctgatggagatggagcccaaggcgctgagcgcccgcaagtgccaggtggtgttcttccgcgtgaaggagatcctgcactgccactccatgttccagatcgccctgtcctcccgcgtggctgagtgggattccaccgagaagatcggggacctcttcgtggcctcgttttccaagtccatggtgctagatgtgtacagtgactacgtgaacaacttcaccagtgccatgtccatcatcaagaaggcctgcctcaccaagcctgccttcctcgagttcctcaagcgacggcaggtgtgcagcccagaccgtgtcaccctctacgggctgatggtcaagcccatccagaggttcccacagttcatactcctgcttcaggacatgctgaagaacacccccaggggccatccggacaggctgtcgctgcagctggccctcacagagctggagacgctggctgagaagctgaacgagcagaagcggctggctgaccaggtggctgagatccagcagctgaccaagagcgtcagtgaccgcagcagcctcaacaagctgttgacctcaggccagcggcagctgctcctgtgtgagacgttgacggagaccgtgtacggtgaccgcgggcagctaattaagtccaaggagcgtcgggtcttcctgctcaacgacatgcttgtctgtgccaacatcaacttcaagcctgccaaccacaggggccagctggagatcagcagcctggtgcccctggggcccaagtatgtggtgaagtggaacacggcgctgccccaggtgcaggtggtggaggtgggccaggacggtggcacctatgacaaggacaatgtgctcatccagcactcaggcgccaagaaggcctctgcctcagggcaggctcagaataaggtgtacctcggccccccacgcctcttccaggagctgcaggacctgcagaaggacctggccgtggtggagcagatcacgcttctcatcagcacgctgcacggcacctaccagaacctgaacatgactgtggctcaagactggtgcctggccctgcagaggctgatgcgggtgaaggaggaagagatccactcggccaacaagtgccgtctcaggctcctgcttcctgggaaacccgacaagtccggccgccccattagcttcatggtggttttcatcacccccaaccccctgagcaagatttcctgggtcaacaggttacatttggccaaaatcggactccgggaggagaaccagccaggctggctatgcccggatgaggacaagaagagcaaagccccattctggtgcccgatcctggcctgctgcatccctgccttctcctcccgggcactcagcctgcagcttggggccctggtccacagtcctgtcaactgtcccctgctgggtttctcagcagtcagcacctcccttccacagggctacctctgggtcgggggcggacaggaaggcgcagggggccaggtggaaatcttttccttgaaccggccctcgccccgcaccgtcaagtccttcccactggcagcccctgtgctctgcatggagtatatcccggagctggaggaggaggcggagagcagagacgagagcccgacagttgctgacccctcggccacggtgcatccaaccatctgcctcgggctccaggatggcagcatcctcctctacagcagtgtggacactggcacccagtgcctggtgagctgcaggagcccaggtctgcagcctgtgctctgcctgcgacacagccccttccacctgctcgctggcctgcaggatgggacccttgctgcttaccctcggaccagcggaggtgtcctgtgggacctggagagccctcccgtgtgcctgactgtggggcccgggcctgtccgcaccctgttgagcctggaggatgccgtgtgggccagctgtgggccccgggtcactgtcctggaagccaccaccctgcagcctcagcaaagcttcgaggcgcaccaggacgaggcagtgagcgtgacacacatggtgaaggcgggcagcggcgtctggatggccttctcctccggcacctccatccgcctcttccacactgagaccctggagcatctgcaagagatcaacatcgccaccaggaccaccttcctcctgccaggccagaagcacttgtgtgtcaccagcctcctgatctgccagggtctgctctgggtgggcactgaccagggtgtcatcgtcctgctgcccgtgcctcggctggaaggcatccccaagatcacagggaaaggcatggtctcactcaacgggcactgtgggcctgtggccttcctggctgtggctaccagcatcctggcccctgacatcctgcggagtgaccaggaggaggctgaggggccccgggctgaggaggacaagccagacgggcaggcacacgagcccatgcccgatagccacgtgggccgagagctgacccgcaagaagggcatcctcttgcagtaccgcctgcgctccaccgcacacctcccgggcccgctgctctccatgcgggagccggcgcctgctgatggcgcagctttggagcacagcgaggaggacggctccatttacgagatggccgacgaccccgacatctgggtgcgcagccggccctgcgcccgcgacgcccaccgcaaggagatttgctctgtggccatcatctccggcgggcagggctaccgcaactttggcagcgctctgggcagcagtgggaggcaggccccgtgtggggagacggacagcaccctcctcatctggcaggtgcccttgatgctatagcgcctcccctctcccctcagagggcacagctgcaggcctgaccaaggccacgcccggctctcgtgctctaggacctgcacgggacttgtggatgggcctggactctccagaaactacttgggcagagcaaaggaaaacctcttgttttaaaaaaatttttttcagagtgttttggggaggagttttagggcttggggagagggaggacacatctggaggaaatggccttctttttaaaagcaaaaaacacaaaacctcacaactgcctggcaagcccagtatcacttgtttgggccctagcgggactccaaggcagccacacgcccctcctggaagggtgtgtgcgtgtgagtgtgtgcgagtgtgtgggctggtgtgtgaatatctataaataagtatatatggtgtatattatatgtgtataaataaagtctgtacatattggagctctgggagatgctggaataaaagacaagagttacatctggacttggaaaaaaaaa

SEQ ID NO: 44 - DNA, 6050 bp, Homo sapiens
atccatggcacggagcggcggcggcggcggcagcaggagcccggcgcgatccgctaggtcccagcccagcgcccagcgagcaggcgacgcggaggggccgggcctccagtgtcccgagggccgggcgctgagactccggccgcgcagctgggagctgcccgcgctgcgctgacagccgcgccgacgtcctccccgccggggcgctcgcaggacatgcccccggggcgcggcggcggggaccccggggctcgcctccgcccagggcccccctccacgccctcgggagccccgggcccccgctgagcactcctcccgcacgcctgggtccctccggccggcgcgcagcccggccccagcgctgtgggtccccgcggggcgatgggttgatgggcgccgggggacgcaggatgcggggggcgcccgcgcgcctgctgctgccgctgctgccgtggctcctgctgctcctggcgcccgaggctcggggcgcgcccggctgcccgctatccatccgcagctgcaagtgctcgggggagcggcccaaggggctgagcggcggcgtccctggcccggctcggcggagggtggtgtgcagcggcggggacctcccggagcctcccgagcccggccttctgcctaacggcaccgttaccctgctcttgagcaataacaagatcacggggctccgcaatggctccttcctgggactgtcactgctggagaagctggacctgaggaacaacatcatcagcacagtgcagccgggcgccttcctgggcctgggggagctgaagcgtttagatctctccaacaaccggattggctgtctcacctccgagaccttccagggcctccccaggcttctccgactaaacatatctggaaacatcttctccagtctgcaacctggggtctttgatgagctgccagcccttaaggttgtggacttgggcaccgagttcctgacctgtgactgccacctgcgctggctgctgccctgggcccagaatcgctccctgcagctgtcggaacacacgctctgtgcttaccccagtgccctgcatgctcaggccctgggcagcctccaggaggcccagctctgctgcgagggggccctggagctgcacacacaccacctcatcccgtccctacgccaagtggtgttccagggggatcggctgcccttccagtgctctgccagctacctgggcaacgacacccgcatccgctggtaccacaaccgagcccctgtggagggtgatgagcaggcgggcatcctcctggccgagagcctcatccacgactgcaccttcatcaccagtgagctgacgctgtctcacatcggcgtgtgggcctcaggcgagtgggagtgcaccgtgtccatggcccaaggcaacgccagcaagaaggtggagatcgtggtgctggagacctctgcctcctactgccccgccgagcgtgttgccaacaaccgcggggacttcaggtggccccgaactctggctggcatcacagcctaccagtcctgcctgcagtatcccttcacctcagtgcccctgggcgggggtgccccgggcacccgagcctcccgccggtgtgaccgtgccggccgctgggagccaggggactactcccactgtctctacaccaacgacatcaccagggtgctgtacaccttcgtgctgatgcccatcaatgcctccaatgcgctgaccctggctcaccagctgcgcgtgtacacagccgaggccgctagcttttcagacatgatggatgtagtctatgtggctcagatgatccagaaatttttgggttatgtcgaccagatcaaagagctggtagaggtgatggtggacatggccagcaacctgatgctggtggacgagcacctgctgtggctggcccagcgcgaggacaaggcctgcagccgcatcgtgggtgccctggagcgcattgggggggccgccctcagcccccatgcccagcacatctcagtgaatgcgaggaacgtggcattggaggcctacctcatcaagccgcacagctacgtgggcctgacctgcacagccttccagaggagggagggaggggtgccgggcacacggccaggaagccctggccagaaccccccacctgagcccgagcccccagctgaccagcagctccgcttccgctgcaccaccgggaggcccaatgtttctctgtcgtccttccacatcaagaacagcgtggccctggcctccatccagctgcccccgagtctattctcatcccttccggctgccctggctcccccggtgcccccagactgcaccctgcaactgctcgtcttccgaaatggccgcctcttccacagccacagcaacacctcccgccctggagctgctgggcctggcaagaggcgtggcgtggccacccccgtcatcttcgcaggaaccagtggctgtggcgtgggaaacctgacagagccagtggccgtttcgctgcggcactgggctgagggagccgaacctgtggccgcttggtggagccaggaggggcccggggaggctgggggctggacctcggagggctgccagctccgctccagccagcccaatgtcagcgccctgcactgccagcacttgggcaatgtggccgtgctcatggagctgagcgcctttcccagggaggtggggggcgccggggcagggctgcaccccgtggtatacccctgcacggccttgctgctgctctgcctcttcgccaccatcatcacctacatcctcaaccacagctccatccgtgtgtcccggaaaggctggcacatgctgctgaacttgtgcttccacatagccatgacctctgctgtctttgcggggggcatcacactcaccaactaccagatggtctgccaggcggtgggcatcaccctgcactactcctccctatccacgctgctctggatgggcgtgaaggcgcgagtgctccataaggagctcacctggagggcaccccctccgcaagaaggggaccccgctctgcctactcccagtcctatgctccggttctatttgatcgctggagggattccactcattatctgtggcatcacagctgcagtcaacatccacaactaccgggaccacagcccctactgctggctggtgtggcgtccaagccttggcgccttctacatccctgtggctttgattctgctcatcacctggatctatttcctgtgcgccgggctacgcttacggggtcctctggcacagaaccccaaggcgggcaacagcagggcctccctggaggcaggggaggagctgaggggttccaccaggctcaggggcagcggccccctcctgagtgactcaggttcccttcttgctactgggagcgcgcgagtggggacgcccgggcccccggaggatggtgacagcctctattctccgggagtccagctaggggcgctggtgaccacgcacttcctgtacttggccatgtgggcctgcggggctctggcagtgtcccagcgctggctgccccgggtggtgtgcagctgcttgtacggggtggcagcctccgccctgggcctcttcgtcttcactcaccactgtgccaggcggagggacgtgagagcctcgtggcgcgcctgctgcccccctgcctctcccgcggccccccatgccccgccccgggccctgcccgccgccgcagaggacggttccccggtgttcggggagggccccccctccctcaagtcctccccaagcggcagcagcggccatccgctggctctgggcccctgcaagctcaccaacctgcagctggcccagagtcaggtgtgcgaggcgggggcggcggccggcggggaaggagagccggagccggcgggcacccggggaaacctcgcccaccgccaccccaacaacgtgcaccacgggcgtcgggcgcacaagagccgggccaagggacaccgcgcgggggaggcctgcggcaagaaccggctcaaggccctgcgcgggggcgcggcgggggcgctggagctgctgtccagcgagagcggcagtctgcacaacagccccaccgacagctacctgggcagcagccgcaacagcccgggcgccggcctgcagctggaaggcgagcccatgctcacgccgtccgagggcagcgacaccagcgccgcgccgctttctgaggcgggccgggcaggccagcgccgcagcgccagccgcgacagtctcaagggcggcggcgcgctggagaaggagagccatcgccgctcgtacccgctcaacgccgccagcctaaacggcgcccccaaggggggcaagtacgacgacgtcaccctgatgggcgcggaggtagccagcggcggctgcatgaagaccggactctggaagagcgaaactaccgtctaaggtggggcgggcgacgcggtagacgggctggccacgcggctcgttcccccgctcctcggggccctccaaggtgtctccgtagtcagcaggttggaggcagaggagccgatggctggaggaagcccacaggcggatgttccccacttgcctagagggcatccctctggggtagcgacagacaatcccagaaacacgcataatacatttccgtccagcccggggcagtctgactgtcggtgccctcccaggaacggggaaggcctccgtctgtgtgaaagggcacagcacatcccaggtgcaccctccccaagtactcccaccccgcctactgtccatgcggcctcactgggggccatcagcctcaccagcaaagcagagatgagagcgtgggaactgtgttctttcctccctgccctctactgatttcagcccagcccctgcctagatcctaggtcccttttcctcccgagtttggctggcacgagagctagcccagcacatgaagcaggtgatgttaagtcacaaggtgctgcttttcagatccactatgcaagaggggagggtggggccacgtgaaaggcagctctagacatcaaccagtcctgggggaggggagtgggaaccgggcacaactaggaacaatgccaccattcccacaggagtggtacttaaaccagacagcagggttcagaggtggcacaccgggacaaagctgaggccctgcacctcaacagctgactgccaggtgcctgtgggtgaactgaggggagtagagggagagggcaggtggaactggggcagaatctagtcatgccctaaagctagtcctgtaaacaatggtgccccagaaagctgcaggtggtgtttggagaagcagttacttttcagttacaagacccatctccctagtctcagccttacaacaccacgggactaaggaagagcacttccttgcctccgtaaggccagaggaagaaccatcccaatcatttgatctccagctccacagtagagagaaacctacaaaatgtcaaaccagcttcccgactcccaggagctcaagccaagcccagaggcagtggctggggtccctgcaggtcatgaggggcctatgcctttactccttttaaacaccagcacccgtcttttccccaacctaaaaccaaccaccagcatttcactacaggaccaaatggaaaccgagggaaccctgggtcttgggaagaacaacaggaaaccaaggtctgacctagggttccctcccagtcttcacatcactctggcctcatcaccaaggtgacagaggacacaggggagggggaaaacccacacacactccttggaatgggtcctgttatttatgcttgctgcacagacatattagaagaaaaaaaaaagctttgtattattcttccacatatgctggctgctgtttacacaccctgccaatgccttagcactggagagctttttgcaatatgctggggaaaggggagggagggaatgaaagtgccaaagaaaacatgtttttaagaactcgggttttatacaatagaatgttttctagcagatgcctcttgttttaatatattaaaattttgcaaagccctttgagctactgccttagtctaaaaaaaaaaaaaaaaa

SEQ ID NO: 45 - DNA, 1973 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacaagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 46 - DNA, 4470 bp, Homo sapiens
gtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttttctcttctctgtctaactttctttttcctatcttttttcttctttgttctgtttacttttgctcatatcatcattcccgctatctttctattaactgaccataacacagaactagttgactatatattatgttgaaattttatggcagctatttatttatttaaattttttgtaacagttttgttttctaataagaaaaatccatgctttttgtagctggttgaaaattcaggaatatgtaaaactttttggtatttaattaaattgattccttttcttaattttaaaaaaaaaaaaaaa

SEQ ID NO: 47 - DNA, 1015 bp, Homo sapiens
ggcggccgctggcacgagtgcagggtaactgagccagggccgctggcgcatttggcctggccgaggccaccccgcgcggccgctccactgtgcccgaggctgtcctggaggtgaggccggcccacagggaccctgcccgtgcccgggctccgggcggattcctgggcaagatgaagtgggtgtgggcgctcttgctgttggcggcgctgggcagcggccgcgcggagcgcgactgccgagtgagcagcttccgagtcaaggagaacttcgacaaggctcgcttctctgggacctggtacgccatggccaagaaggaccccgagggcctctttctgcaggacaacatcgtcgcggagttctccgtggacgagaccggccagatgagcgccacagccaagggccgagtccgtcttttgaataactgggacgtgtgcgcagacatggtgggcaccttcacagacaccgaggaccctgccaagttcaagatgaagtactggggcgtagcctcctttctccagaaaggaaatgatgaccactggatcgtcgacacagactacgacacgtatgccgtgcagtactcctgccgcctcctgaacctcgatggcacctgtgctgacagctactccttcgtgttttcccgggaccccaacggcctgcccccagaagcgcagaagattgtaaggcagcggcaggaggagctgtgcctggccaggcagtacaggctgatcgtccacaacggttactgcgatggcagatcagaaagaaaccttttgtagcaatatcaagaatctagtttcatctgagaacttctgattagctctcagtcttcagctctatttatcttaggagtttaatttgcccttctctccccatcttccctcagttcccataaaaccttcattacacataaagatacacgtgggggtcagtgaatctgcttgcctttcctgaaagtttctggggcttaagattccagactctgattcattaaactatagtcacccgtgtcctgtga

SEQ ID NO: 48 - DNA, 2851 bp, Homo sapiens
aaggcggagccagcggcggcccaggctctgggcactcgcgcggctcccgttcgcggagcacacatcccggcgcgacgaggttccttcggagagcgagcgggagtcggtgctcggcctcctgcggggagcagcccgaggaatctgcagggagaggtcgggaggtgacaacgccagcatgccagtgctatcggaggactctggtttgcatgaaaccctggccctgctgacctcccagctcagacctgactccaaccacaaggaggagatgggcttcctgagggatgttttcagtgaaaaaagcctcagttacttaatgaagattcatgagaagcttcgctattatgaaaggcaaagtccaaccccagttctgcacagcgctgtggccctcgctgaggacgtgatggaggagttgcaggccgcctccgtgcacagtgatgagagggagctgctccagctgctgtccaccccgcacctgagggctgtgctcatggtacatgacacggttgcccagaagaattttgaccccgttctcccgcctctgcctgacaatatcgatgaggattttgatgaggaatcggtgaagatcgtccgcttggtgaagaacaaggaacccctgggtgccaccatccggcgggacgagcactcaggggctgttgtggtggccaggatcatgcgaggaggcgcagcagacaggagcggcctggtccacgttggagatgagctccgagaagtgaacgggatcgcagtcctgcacaagcggcccgacgagatcagccagattctggcccagtcccagggatccatcaccctaaaaatcatcccagccacccaggaggaagatcgcttaaaggagagcaaggtgttcatgcgcgccctcttccactacaaccctcgggaggaccgggccatcccttgccaggaggcgggcctgcccttccagcgcaggcaggtcctggaggtggtgagccaggacgaccccacgtggtggcaggccaagcgagtcggggacaccaaccttcgagccggcctcatcccctccaaggggttccaggagagacgactaagctaccggagagccgcgggcaccctgccgagcccccagagcctcaggaagcccccctatgatcagccttgtgacaaagagacctgtgactgtgagggctacctcaaagggcactatgtggctggtcttcggaggagcttccggctgggctgtagggagagactgggtggctcgcaggaaggaaagatgtcctccggagctgagtctccggagctgctgacttacgaagaggtggccaggtaccaacaccagcccggagagcggccccgcctggtggttctgatcgggtctctgggagcccgactgcacgagctgaagcaaaaggtggtggctgagaacccacagcactttggcgtcgctgttccacataccaccaggccccgaaagagccatgagaaggaaggagtggaatatcactttgtgtctaagcaagcatttgaggccgacttacatcacaacaagttcctggaacatggtgaatataaggaaaatctgtatggaaccagcctggaggccattcaggctgttatggccaaaaacaaagtttgtttggtggatgtggagccagaagcactgaaacaactgaggacctcagaatttaaaccctatattatatttgtaaagcctgcaattcaggaaaaaagaaaaacgccacctatgtccccagcttgtgaggacacagcagccccatttgatgagcagcagcaagagatggccgcttctgccgccttcatagaccggcattacgggcacctggtagacgccgtgctggtgaaggaggatctccagggtgcctacagccagctcaaagtggtcttagagaagctgagcaaggacactcactgggtacctgttagttgggtcaggtaactttatcccagaacatccaagctggacgggaccttgaagatcatctagtccagactccctcattttaccatcaaggaatctcaagcgcagagagggagagaattctccacaaattccatcatcgagaagagtataagtgggaagtcttgtttgttgttggtttttgtctgttgtttttcactgcacctctttggatcatgatttgaaaggggcatatcagaaaacaacacatttcatttattaaagtatcacaggcaagctgaccctgattctttgtaccaaagttaagtagccactgtcttttgtgggtggtagtggttaatttatacagtactgattcgcagaatgtttaagctttttaaacatagtgacgcttagtagtttttttggaagctaacttgttttatccaaggggattttacatgtaactgaagttcccctgtcttcaagcactaaaacgttgatcttaaccttttttttgaagtgcttgcctggtaatagaaaacgggttctctgcctattttaaaatagtgaatatacgtaaattttctctggaaggctgaggcacacttcaccatcaacatgaattactgtactatcctgtactgcagtggtgccttcagggactcgaggaatgtaaggttgcctttcccctttctaaataccctcagattcctaacatcgagcccatgctttgtttgatttgttctattccatccattgtcccttttgttactgacagttgccttggtcctagccagtccctgccatgagatcataggggttcccattgtgctagatcttgggaaaccagatgactctccctgtcaaaactatggctacgtcactgtaaaccatttctgtcaagaataaaagtatgtagacccagagtgtgggcctaaaaaaaaaaaaaaaaaa

SEQ ID NO: 49 - DNA, 2896 bp, Homo sapiens
acgcttgcgcgcgggatttaaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaaaaaaaaaaaaaaaaa

SEQ ID NO: 50 - DNA, 10275 bp, Homo sapiens
actcccaccctaagtgctgcagactcttccctgaagctgccggctgaggccggagctgccgcctccatgagaggcttcctcctacaccccagggccagaggaccctttgccaccagagtgagatcctagagaccatcatcctggtaaatcccagtgcagacagcatcagctctgaggttcatcatcttcttagcagctcatcagcttataaactactaatcttgagtgggcaaagtttagagcctgggggagacctcatcctacagagtggcacctactcatatgaaaactttgcccaggtccttcacaaccccgagatttcccaattgctcagcaatagagaccctgggatacaggccttccttaccgtgtcctgcttaggggaaggtgattggagccacctgggattatccagttcccaagagaccctgcacctccggctaaaccctgagcccactctgcccaccatggacggcgtggctgagttctccgagtatgtctctgagactgtggacgtgccatccccatttgacctactagagccccccacctcagggggcttcctcaagctctccaagccttgttgctacatcttcccaggtggtcgtggggactctgccctctttgctgtcaatggtttcaacatcctggtggatggtggctctgatcgcaagtcctgtttttggaagctggtacggcacttggaccgcattgactcggtgctactcacacacattggggcagacaacctgccaggcatcaatggactactgcagcgcaaagtggcagagctagaggaggagcagtcccagggctctagcagttacagcgactgggtgaagaaccttatctctcctgagcttggagttgtctttttcaacgtgcctgagaagctgcggcttcctgatgcctcccggaaagccaagcgtagcattgaggaggcctgcctcactctgcagcacttaaaccgcctgggcatccaggctgagcctctatatcgtgtggtcagcaataccattgagccactgaccctcttccacaaaatgggtgtgggccggctggacatgtatgtcctcaaccctgtcaaggacagcaaggagatgcagttcctcatgcaaaagtgggcaggcaatagtaaagccaagacaggcatcgtgctgcccaatgggaaggaggctgagatctccgtgccctaccttacctctatcactgctctggtggtctggctaccagccaatcccactgagaagattgtgcgtgtgctttttccaggaaatgctccccaaaacaagatcttggagggcctagaaaagcttcggcatctggacttcctgcgttaccctgtggccacgcagaaggacctggcttctggggctgtgcctaccaacctcaagcccagcaaaatcaaacagcgggctgatagcaaggagagcctcaaagccactaccaagacggccgtgagcaagttggccaaacgggaggaggtggtagaagagggagccaaggaggcacgttcagagctggccaaggagttagccaagacagagaagaaggcaaaagagtcatctgagaagcccccagagaagcctgccaagcctgagagggtgaagacagagtcaagtgaggcactgaaggcagagaagcgaaagctgatcaaagacaaggtagggaaaaagcaccttaaagaaaagatatcaaagctggaagaaaaaaaagacaaggagaaaaaagagatcaaaaaggagaggaaagagctcaagaaggatgaaggaaggaaggaggagaagaaggatgccaagaaggaggagaagaggaaagataccaaacctgagctcaagaagatttccaagccagacctaaagccctttactcctgaggtacgtaagaccctctataaagccaaggtccctggaagagtcaaaatagacaggagccgtgctatccgtggggagaaggagctgtcttctgagccccagacacccccagcccagaagggaactgtaccactcccaaccatcagtgggcacagggagctggtcctatcctcaccagaggacctcacacaggactttgaggagatgaagcgtgaggagagggctttgctggctgaacaaagggacacaggactaggagataagccattccctctagacactgcagaggagggacccccaagtacagctatccagggaacaccaccctctgttccagggctgggacaagaagaacatgtgatgaaggagaaagagcttgtcccagaggtccctgaggaacaaggcagcaaggacagaggcctagactctggggctgaaacagaggaagagaaagatacctgggaggaaaagaagcagagggaagcagagaggctcccagacagaacagaagccagagaggaaagtgaacctgaagtaaaggaggatgtgatagaaaaggctgagttagaagaaatggaggaggtacacccttcagatgaggaggaagaggacgcgacaaaagctgagggtttttaccaaaaacatatgcaggaacccttgaaggtaactccaaggagccgggaggcttttgggggtcgggaattgggactccagggcaaggcccctgagaaggagacctcgttattcctaagcagcctgaccacacctgcaggagccactgagcatgtctcttacatccaggatgagacaatccctggctactcagagactgagcagaccatctcagatgaggagatccatgatgagccggaggagcgcccagctccacccagatttcatacaagtacatatgacctgcccgggcctgaaggtgctggcccattcgaagccagccaacctgccgatagtgctgttcctgctacctctggcaaagtctatggaacgccagagactgaactcacctaccccactaacatagtggctgcccctttggctgaagaggaacatgtgtcctcggccacttcaatcactgagtgtgacaaactttcttcctttgccacatcagtggctgaggaccaatctgtggcctcacttacagctccccagacagaggagacaggcaagagctccctgctgcttgacacagtcacaagcatcccttcctcccgtactgaagctacgcagggcttggactatgtgccatcagctggtaccatctcacccacctcctcactggaagaagacaagggcttcaaatcaccaccctgtgaggacttctctgtgactggggagtcagagaagagaggagagatcatagggaaaggcttgtctggagagagagctgtggaagaggaagaggaggagacagcaaacgtagagatgtctgagaaactttgcagtcaatatggaactccagtgtttagtgcccctgggcatgccctacatccaggagaaccagcccttggagaagcagaggagcggtgccttagcccagatgacagcacagtgaagatggcttctcctccaccatctggcccacccagtgccacccacacaccctttcatcagtccccagtggaagaaaagtctgagccccaagactttcaggaggcagactcctggggagacactaagcgcacaccaggtgtgggcaaagaagatgctgctgaggagacagtcaagccagggcctgaagagggcacactagagaaggaagagaaagttcctcctcccaggagcccccaggcccaggaagcacctgtcaacattgatgaggggcttacaggctgtaccattcaactgttgccagcacaggataaagcaatagtctttgagattatggaggcaggagagcccacaggcccaattctgggagcagaagcccttcccggaggtttgaggactttaccccaagaacctggcaaacctcagaaagatgaggtgctcagatatcctgaccgaagcctctctcctgaagatgcagaatccctctctgtcctcagcgtgccctccccagacactgccaaccaagagcctacccccaagtctccctgtggcctgacagaacagtacctacacaaagaccgttggccagaggtatctccagaagacacccagtcactttctctgtcagaagagagtcccagcaaggagacctccctggatgtctcttctaagcagctctctccagaaagccttggcaccctccagtttggggaactaaaccttgggaaggaagaaatggggcatctgatgcaggccgaggatacctctcaccacacagctcccatgtctgttccagagccccatgcagccacagcgtcacctcccacagatgggacaactcgatactctgcacagacagacatcacagatgacagccttgacaggaagtcacctgccagctcattctctcactctacaccttcaggaaatgggaagtacttacctggggcgatcacaagccctgatgaacacattctgacacctgatagctccttctccaagagtcctgagtctttgccaggccctgccttggaggacattgccataaagtgggaagataaagttccagggttgaaagacagaacctcagaacagaagaaggaacctgagccaaaggatgaagttttacagcagaaagacaaaactctggagcacaaggaggtggtagagccgaaggatacagccatctatcagaaagatgaggctctgcatgtaaagaatgaggctgtgaaacagcaggataaggctttagaacaaaagggcagagacttagagcaaaaagacacagccctagaacagaaggacaaggccctggaaccaaaagacaaagacttagaagaaaaagacaaggccctggaacagaaggataagattccagaagagaaagacaaagccttagaacaaaaggatacagccctggaacagaaggacaaggccctggaaccaaaagataaagacttggaacaaaaggacagggtcctagaacagaaggagaagatcccagaagagaaagacaaagccttagatcaaaaagtcagaagtgttgaacataaggctccggaggacacggtcgctgaaatgaaggacagagacctagaacagacagacaaagcccctgaacagaaacaccaggcccaggaacaaaaggataaagtctcagaaaagaaggatcaggccttagaacaaaaatactgggctttgggacagaaggatgaagccctggaacaaaacattcaggctctggaagagaaccaccaaactcaggagcaggagagcctagtgcaggaggataaaaccaggaaaccaaagatgctagaggaaaaatccccagaaaaggtcaaggccatggaagagaagttagaagctcttctggagaagaccaaagctctgggcctggaagagagcctagtgcaggagggcagggccagagagcaggaagaaaagtactggagggggcaggatgtggtccaggagtggcaagaaacatctcctaccagagaggagccggctggagaacagaaagagcttgccccggcatgggaggacacatctcctgagcaggacaataggtattggaggggcagagaggatgtggccttggaacaggacacatactggagggagctaagctgtgagcggaaggtctggttccctcacgagctggatggccagggggcccgcccacactacactgaggaacgggaaagcactttcctagatgagggcccagatgatgagcaagaagtacccctgcgggaacacgcaacccggagcccctgggcctcagacttcaaggatttccaggaatcctcaccacagaaggggctagaggtggagcgctggcttgctgaatcaccagttgggttgccaccagaggaagaggacaaactgacccgctctccctttgagatcatctcccctccagcttccccacctgagatggttggacaaagggttccttcagccccaggacaagagagtcctatcccagaccctaagctcatgccacacatgaagaatgaacccactactccctcatggctggctgacatcccaccctgggtgcccaaggacagacccctcccccctgcacccctctccccagctcctggtccccccacacctgccccggaatcccatactcctgcacccttctcttggggcacagccgagtatgacagtgtggtggctgcagtgcaggagggggcagctgagttggaaggtgggccatactcccccctggggaaggactaccgcaaggctgaaggggaaagggaagaagaaggtagggctgaggctcctgacaaaagctcacacagctcaaaggtaccagaggccagcaaaagccatgccaccacggagcctgagcagactgagccggagcagagagagcccacaccctatcctgatgagagaagctttcagtatgcagacatctatgagcagatgatgcttactgggcttggccctgcatgccccactagagagcctccacttggagcagctggggattggcccccatgcctctcaaccaaggaggcagctgccggccgaaacacatctgcagagaaggagctttcatctcctatctcacccaagagcctccagtctgacactccaaccttcagctatgcagccctggcaggacccactgtacccccaaggccagagccagggccaagtatggagcccagcctcaccccacctgcagttcccccccgtgctcctatcctgagcaaaggcccaagcccccctcttaatggtaacatcctgagctgcagcccagataggaggtccccatcccccaaggaatcaggccggagtcactgggatgacagcactagtgactcagaactggagaagggggctcgggaacagccagaaaaagaggcccaatccccaagtcctcctcaccccattcctatggggtcccccacattatggccagaaactgaggcacatgttagccctcccttggactcacacctggggcctgcccgacccagtctggacttccctgcttcagcctttggcttctcctcattgcagccagctcccccacagctgccctctccagctgaaccccgctcggcaccctgtggctcccttgccttctctggggatcgagctctggctctggctccaggaccccccaccagaacccggcatgatgaatacctggaagtgaccaaggcccccagcctggattcctcactgccccagctcccatcacccagttctcctggggcccctctcctctccaatctgccacgacctgcctcaccagccctgtctgagggctcctcctctgaggctaccacgcctgtgatttcaagtgtggcggagcgcttctctccaagccttgaggctgcagaacaggagtctggagagctggacccaggaatggaaccagctgcccacagcctctgggacctcactcctctgagcccagcacccccagcttcactggacttggccctagctccagctccaagcctgcctggagacatgggtgatggcatcctgccgtgccacctggagtgctcagaggcagccacggagaagccaagccccttccaggttccctctgaggattgtgcagccaatggcccaactgaaaccagccctaaccccccaggccctgccccagccaaggctgaaaatgaagaggctgcggcttgccctgcctgggaacgtggggcctggcctgaaggagctgagaggagctcccggcctgacacattgctctcccctgagcagccagtgtgtcctgcagggggctccgggggcccacccagcagtgcctctcctgaggtcgaagctgggccccagggatgtgccactgagcctcggccccatcgtggggagctctccccatccttcctgaacccacctctgcccccatccatagatgatagggacctctcaactgaggaagttcggctagtaggaagaggggggcggcgccgggtaggggggccagggaccactgggggcccatgccctgtgactgatgagacaccccctacatcagccagtgactcaggctcctcacagtcagattctgatgtcccgccagaaactgaggagtgtccgtccatcacagctgaggcagccctcgactcagatgaagatggagacttcctacctgtggacaaagctgggggtgtcagtggtactcaccaccccaggcctggccatgacccacctcctctcccacagccagacccccgcccatcccctccccgccctgatgtgtgcatggctgaccccgaggggctcagctcagagtctgggagagtagagaggctacgggagaaggaaaaggttcaggggcgagtagggcgcagggccccaggcaaggccaagccagcgtcccctgcacggcgtctggatcttcggggaaaacgctcacccacccctggtaaagggcctgcagatcgagcatcccgggccccacctcgaccacgcagcaccacaagccaggtcaccccagcagaggaaaaggatggacacagccccatgtccaaaggcctagtcaatggactcaaggcaggaccaatggccttgagttccaagggcagctctggtgcccctgtatatgtggatctcgcctacatcccgaatcattgcagtggcaagactgctgaccttgacttcttccgtcgagtgcgtgcatcctactatgtggtcagtgggaatgaccctgccaatggcgagccaagccgggctgtgctggatgccctgctggagggcaaggcccagtggggggagaatcttcaggtgactctgatccctactcatgacacggaggtgactcgtgagtggtaccaacaaactcatgagcagcagcaacaactgaatgtcctggtcctggctagcagcagcaccgtggtgatgcaggatgagtccttccctgcctgcaagattgagttctgaaagagccgccctcccttccccaaggatccactcccccagctcctttagagaatggctactgctgagtcctttggggttgagggagatgggagctagggggaggggagggagatgtcttgttgtggggacttgggctgggctaaatgggaggggttgtccctccccatcatccattcctgtgaggtgtctcaaaccaaagttaacagggagaggatgggggaggggacaaattagaataggatagcatctgatgcctgagaaccctctcctagcactgtcaaatgctggtattgaatggggactgaggatgggtctcagagagcaacctcctccctcgtagagggagattatatccccaactccagggacctctttatctcaatctatttatttggcatcctgggagggatttccaatagtaatttatgtgacctggggcaggataccgtcagtgaggtgcccagagctgcaccctttcctccatttcccatcccccatctcctcaaccaccagggtctgagttctagcagggtcctgggggtatcccactgctatactgttctactgcttccctcagtatctgaatgtctcaatttaaaacttgaagctctttagaccaatagactggtgagaggagaaaggagcttatcccccagaccctgctttataccattcacatcccagggctgtgtccagacagcacaaaacggcaaggagagcccaagccccaatgccagaattcttccaaactccctgactctttgaagtttttactcaccccatttcaattatcctgatcccttctcatcccctgcttggcttctctgcatgtggtcatctgctgtggcttggtgtttaatgggttaaaaataagccactgcctgacatcccaacatttgacaccccagcaatgtgtgactcccccaacattccactatgccatcctgcagctgaaatgggaacactggctgcctctccaaacccgctcttggacagaggatctgggaggtggaagccaggccagaggacttggggaaaatgagatggaggaaggaaaaagggagaagctgagccacagcttaactcctacagagtgaaatgaaaacgggctgaaaataccaccccaggagaggacctcgccccaagcaagccagtgagcagccctgccagactactgccagactgagaaacccagaagctggtagtcatgtgggcttgccttctctgccaaacgactgggaaaccaaaatgagcccaccttgtgttcttcctagctccaccctccccgtgctgctgtgttctgctcctccccacgcttccctgctatagttcccagctgctgtaacggagccacctccaactctaacaataaaccaagttcattgcagatagtgta

SEQ ID NO: 51 - DNA, 3185 bp, Homo sapiens
ggcagtgagactgtaagcagtctgggttgggcagaaggcagaaaaccagcagagtcacagaggagatggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgacctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctgtgcagtgtcagcacctggaagcccccagtgaaggaaccatggactgtgttcatccgctcactgcttttgcctatggctccagctgtaaatttgagtgccagcccggctacagagtgaggggcttggacatgctccgctgcattgactctggacactggtctgcacccttgccaacctgtgaggctatttcgtgtgagccgctggagagtcctgtccacggaagcatggattgctctccatccttgagagcgtttcagtatgacaccaactgtagcttccgctgtgctgaaggtttcatgctgagaggagccgatatagttcggtgtgataacttgggacagtggacagcaccagccccagtctgtcaagctttgcagtgccaggatctcccagttccaaatgaggcccgggtgaactgctcccaccccttcggtgcctttaggtaccagtcagtctgcagcttcacctgcaatgaaggcttgctcctggtgggagcaagtgtgctacagtgcttggctactggaaactggaattctgttcctccagaatgccaagccattccctgcacacctttgctaagccctcagaatggaacaatgacctgtgttcaacctcttggaagttccagttataaatccacatgtcaattcatctgtgacgagggatattctttgtctggaccagaaagattggattgtactcgatcgggacgctggacagactccccaccaatgtgtgaagccatcaagtgcccagaactctttgccccagagcagggcagcctggattgttctgacactcgtggagaattcaatgttggctccacctgccatttctcttgtgacaacggctttaagctggaggggcccaataatgtggaatgcacaacttctggaagatggtcagctactccaccaacctgcaaaggcatagcatcacttcctactccaggggtgcaatgtccagccctcaccactcctgggcagggaaccatgtactgtaggcatcatccgggaacctttggttttaataccacttgttactttggctgcaacgctggattcacactcataggagacagcactctcagctgcagaccttcaggacaatggacagcagtaactccagcatgcagagctgtgaaatgctcagaactacatgttaataagccaatagcgatgaactgctccaacctctggggaaacttcagttatggatcaatctgctctttccattgtctagagggccagttacttaatggctctgcacaaacagcatgccaagagaatggccactggtcaactaccgtgccaacctgccaagcaggaccattgactatccaggaagccctgacttactttggtggagcggtggcttctacgataggtctgataatgggtgggacgctcctggctttgctaagaaagcgtttcagacaaaaagatgatgggaaatgccccttgaatcctcacagccacctaggaacatatggagtttttacaaacgctgcatttgacccgagtccttaaggtttccataaacacccatgaatcaaagacatggaattaccttagattagctctggaccagcctgttggacccgctctggaccaaccctgtttcctgagtttgggattgtggtacaatctcaaattctcaacctaccaccccttcctgtcccacctcttctcttcctgtaacacaagccacagaagccaggagcaaatgtttctgcagtagtctctgtgctttgactcacctgttacttgaaataccagtgaaccaaagagactggagcatctgactcacaagaagaccagactgtggagaaataaaaatacctctttattttttgattgaaggaaggttttctccactttgttggaaagcaggtggcatctctaattggaagaaattcctgtagcatcttctggagtctccagtggttgctgttgatgaggcctcttggacctctgctctgaggcttccagagagtcctctggatggcaccagaggctgcagaaggccaagaatcaagctagaaggccacatgtcaccgtggaccttcctgccaccagtcactgtccctcaaatgacccaaagaccaatattcaaatgcgtaattaaaagaattttccccaaaaaaaaaaaaaaaa

SEQ ID NO: 52 - DNA, 3405 bp, Homo sapiens
aacagctgcagccggcgctgggcccgcctggaatgcgggaacaggctgcacaccaggacttttatggaaacttgctgctggagacggcggcggcggcggcggcagcggcagccagaggactcccagcggctggagcagaagtgttagcggccagagctcccagacccctacccacagccaggcgggacgcgcacagtccctccacgcggaaagaagtaccttcgccggtcaccggctcctgcagggtgcaaatatatacagagcttcataatcagcccaagaccacatagagcaaacatgaatgatatttcccaaaaggctgagattctgctttcttcatctaaacctgtcccaaaaacctatgtaccaaaacttggcaagggtgatgtaaaggataagtttgaagccatgcagagagccagggaagaaagaaatcaaaggagatctagagacgaaaaacaaagaagaaaagaacaatatattagagagagagaatggaacaggagaaagcaggagattaaagaaatgcttgcttctgatgatgaggaagatgtatcttctaaagtagaaaaggcttatgttccaaaattaacaggaactgtgaagggtagatttgctgaaatggagaaacaaagacaagaggaacaaaggaagagaacggaggaggaacgaaaacgcagaattgagcaggatatgttagaaaagaggaaaatacagcgtgaattagcaaaaagggctgaacagattgaggacataaacaatacgggaactgaatcagcatcagaggaaggagatgattcactacttataactgtggtacctgtcaaatcatataaaacatctggaaaaatgaaaaagaattttgaggatctagaaaaagaacgtgaagagaaagaaaggatcaagtacgaggaagataaaagaataagatatgaagaacaacgaccatctctcaaggaagcaaagtgtctttcattagttatggatgatgaaatagaaagtgaagcaaaaaaagaatcactttctcccggaaaattgaaactaacttttgaagaactggagcgacaaagacaagaaaaccgaaagaagcaagctgaagaggaagcaagaaaacgtttagaagaagagaagcgtgcttttgaagaagcaaggcggcaaatggtaaatgaagatgaggaaaaccaagacacagcaaaaatttttaaagggtaccgccctggtaaactcaaactcagttttgaagaaatggaaaggcaaagaagagaagatgaaaaaaggaaagcagaagaagaagccagaaggagaatagaggaagaaaagaaggcgtttgctgaagcaaggagaaatatggtagtagatgatgactccccagagatgtataagacaatctctcaagaatttcttacaccgggaaaactggaaattaattttgaagaattattaaaacaaaaaatggaagaagaaaaacgacgaacagaggaggaacggaagcataagctagaaatggagaaacaagaatttgaacaactgagacaggaaatgggagaggaagaggaagaaaatgaaacctttggattgagcagagaatatgaagaactgatcaaattaaaaaggagtggctctattcaagctaaaaacctaaaaagcaagtttgaaaaaattggacagttgtctgaaaaagaaatacagaaaaaaatagaagaagagcgagcaagaaggagagcaattgaccttgaaattaaagagcgagaagctgaaaattttcatgaggaagatgatgttgatgttaggcctgcaagaaaaagcgaggctccatttactcacaaagtgaatatgaaagctagatttgaacaaatggctaaggcaagagaagaagaagaacaaagaagaattgaagaacaaaagttactacgcatgcagtttgaacaaagggaaattgatgcagcactacaaaagaaaagagaagaggaggaggaggaagaaggtagcatcatgaatggctccactgctgaagatgaagagcaaaccagatcaggagctccatggttcaagaagcctcttaaaaacacatcagttgtagacagtgagccagtcagatttacggttaaagtaacaggagaacccaaaccagaaattacatggtggtttgaaggagaaatactgcaggatggagaagactatcaatatattgaaaggggagaaacttactgcctttacttaccagaaactttcccagaagatggaggagagtatatgtgtaaagcagtcaacaataaaggatctgcagctagtacctgtattcttaccattgaaagtaagaattaatcactctttttatcttttattctattaatttttttttccttaaaatcacttttcttcttctcttttttagctgatgactactagctcccctcccctctccctggaactttctctttcactccaactttcttactacatccatcttttctgtggcggggccaaaaaaggaaaccaggagtgccactatgctgacttcttattccttttcataacagtcttcaaagcacagctcatctaaagaatgcctacttcttttccaaataagcatcagatttatcgcctattatgcagtaacagtcaataaaatgtacttatgggggggaattactcaattattctatcagaacctattataaagactgtatttcccatagacgtttacagcaactatgtttaaaaaacaaaaacaaaaaaaaaacacacaaacctaagtagaatacattattttgcatgaaggaatgtcatttctgagctttttacacctaaaattaggctgaaatagctgagataattaatttggaacctatcaatttgagtggactttttctttagtagtacaccattttggttgttgtagtttcaaagtctttctgaagcagatatattgggattggagcggggtggggaaaactgtcactcctttcagaggaaaaggggaggagcatggagaaaaacaaaaattaaaggacttaaagaatggctatacagtgttgagtgttgaggatattaaacatgttatttttcaaacgtatgtaatatatattaaatttataaagcaaatttatgttgtgatcttgcctgaacaaattatattttaatgaaaaaactttctattaatagttcacgcaagagaaaacactttcaacatagtcgaaggcttcaagatctaagtgtatcagacttagggaaaaagtggcacaaccttcgatttaaaattctagtctttaaaatgagtttgtaaataattagctattacgttctattaagttgttttatattttaattttctggaagacaattttattttacaacgtgaacccaaataaagtaacttctgtatttaaaaaaaaaaaaaaaaaa

SEQ ID NO: 53 - DNA, 3449 bp, Homo sapiens
gctctgcccgttgctcagcaagttacttggggttccagtttgataagaaaagacttcctgtggaggaatctgaagggaaggaggaggagctggcccattcctgcctgggaggttgtggaagaaggaagatggccagagctttgtgtccactgcaagccctctggcttctggagtgggtgctgctgctcttgggaccttgtgctgcccctccagcctgggccttgaacctggacccagtgcagctcaccttctatgcaggccccaatggcagccagtttggattttcactggacttccacaaggacagccatgggagagtggccatcgtggtgggcgccccgcggaccctgggccccagccaggaggagacgggcggcgtgttcctgtgcccctggagggccgagggcggccagtgcccctcgctgctctttgacctccgtgatgagacccgaaatgtaggctcccaaactttacaaaccttcaaggcccgccaaggactgggggcgtcggtcgtcagctggagcgacgtcattgtggcctgcgccccctggcagcactggaacgtcctagaaaagactgaggaggctgagaagacgcccgtaggtagctgctttttggctcagccagagagcggccgccgcgccgagtactccccctgtcgcgggaacaccctgagccgcatttacgtggaaaatgattttagctgggacaagcgttactgtgaagcgggcttcagctccgtggtcactcaggccggagagctggtgcttggggctcctggcggctattatttcttaggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcggagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcagaccaagacacccgtgagctgcttcaacatccagatgtgtgttggagccactgggcacaacattcctcagaagctatccctaaatgccgagctgcagctggaccggcagaagccccgccagggccggcgggtgctgctgctgggctctcaacaggcaggcaccaccctgaacctggatctgggcggaaagcacagccccatctgccacaccaccatggccttccttcgagatgaggcagacttccgggacaagctgagccccattgtgctcagcctcaatgtgtccctaccgcccacggaggctggaatggcccctgctgtcgtgctgcatggagacacccatgtgcaggagcagacacgaatcgtcctggactgtggggaagatgacgtatgtgtgccccagcttcagctcactgccagcgtgacgggctccccgctcctagttggggcagataatgtcctggagctgcagatggacgcagccaacgagggcgagggggcctatgaagcagagctggccgtgcacctgccccagggcgcccactacatgcgggccctaagcaatgtcgagggctttgagagactcatctgtaatcagaagaaggagaatgagaccagggtggtgctgtgtgagctgggcaaccccatgaagaagaacgcccagataggaatcgcgatgttggtgagcgtggggaatctggaagaggctggggagtctgtgtccttccagctgcagatacggagcaagaacagccagaatccaaacagcaagattgtgctgctggacgtgccggtccgggcagaggcccaagtggagctgcgagggaactcctttccagcctccctggtggtggcagcagaagaaggtgagagggagcagaacagcttggacagctggggacccaaagtggagcacacctatgagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtggactgggggctgcccatccccagcccctcccccattcacccggcccatcacaagcgggatcgcagacagatcttcctgccagagcccgagcagccctcgaggcttcaggatccagttctcgtaagctgcgactcggcgccctgtactgtggtgcagtgtgacctgcaggagatggcgcgcgggcagcgggccatggtcacggtgctggccttcctgtggctgcccagcctctaccagaggcctctggatcagtttgtgctgcagtcgcacgcatggttcaacgtgtcctccctcccctatgcggtgcccccgctcagcctgccccgaggggaagctcaggtgtggacacagctgctccgggccttggaggagagggccattccaatctggtgggtgctggtgggtgtgctgggtggcctgctgctgctcaccatcctggtcctggccatgtggaaggtcggcttcttcaagcggaaccggccacccctggaagaagatgatgaagagggggagtgatggtgcagcctacactattctagcaggagggttgggcgtgctacctgcaccgccccttctccaacaagttgcctccaagctttgggttggagctgttccattgggtcctcttggtgtcgtttccctcccaacagagctgggctaccccccctcctgctgcctaataaagagactgagccctgaaaaaaaaaaaaaaaaaaa

SEQ ID NO: 54 - DNA, 1054 bp, Homo sapiens
gcccccgcctggagtccagacccgacggccggcccagttccacgcacccagcgagcccaagcgccttctccgcaccagggaagccccacccaccagaagccaagatgtccagcaagcgggccaaagccaagaccaccaagaagcggccacagcgggccacatccaatgtcttcgcaatgtttgaccagtcccagatccaggagtttaaggaggctttcaacatgattgaccagaaccgtgatggcttcattgacaaggaggacctgcacgacatgctggcctcgctgggtttcatccatgaggaccacctccgggagctgctcaccaccatgggtgaccgcttcacagatgaggaagtggacgagatgtaccgggaggcacccattgataagaaaggcaacttcaactacgtggagttcacccgcatcctcaaacatggcgccaaggataaagacgactaggccaccccagccccctgacaccccagcccccgccagtcacccctccccgcacacacccgtccataccagctccctgcccatgaccctcgctcagggatccccctttgaggggttagggtcccagttcccagtggaagaaacaggccaggagaagtgcgtgccgagctgaggcagatgttcccacagtgaccccagagccctgggctatagtctctgacccctccaaggaaagaccaccttctggggacatgggctggagggcaggacctagaggcaccaagggaaggccccattccggggctgttccccgaggaggaagggaaggggctctgtgtgccccccaggaggaagaggccctgagtcctgggatcagacaccccttcacgtgtatccccacacaaatgcaagctcaccaaggtcccctctcagtccccttccctacaccctgaccggccactgccgcacacccacccagagcacgccacccgccatgggagtgtgctcaggagtcgcgggcagcgtggacatctgtcccagagggggcagaatctccaatagaggactgagcactgctaaaaaaaaaaaaaaaaaaa

SEQ ID NO: 55 - DNA, 4894 bp, Homo sapiens
cgccgcgggaggcggacgagatgcgagcgcggccgcggccccggccgctctgggcgactgtgctggcgctgggggcgctggcgggcgttggcgtaggagggcccaacatctgtaccacgcgaggtgtgagctcctgccagcagtgcctggctgtgagccccatgtgtgcctggtgctctgatgaggccctgcctctgggctcacctcgctgtgacctgaaggagaatctgctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccagatgattcgaagaatttctccatccaagtgcggcaggtggaggattaccctgtggacatctactacttgatggacctgtcttactccatgaaggatgatctgtggagcatccagaacctgggtaccaagctggccacccagatgcgaaagctcaccagtaacctgcggattggcttcggggcatttgtggacaagcctgtgtcaccatacatgtatatctccccaccagaggccctcgaaaacccctgctatgatatgaagaccacctgcttgcccatgtttggctacaaacacgtgctgacgctaactgaccaggtgacccgcttcaatgaggaagtgaagaagcagagtgtgtcacggaaccgagatgccccagagggtggctttgatgccatcatgcaggctacagtctgtgatgaaaagattggctggaggaatgatgcatcccacttgctggtgtttaccactgatgccaagactcatatagcattggacggaaggctggcaggcattgtccagcctaatgacgggcagtgtcatgttggtagtgacaatcattactctgcctccactaccatggattatccctctttggggctgatgactgagaagctatcccagaaaaacatcaatttgatctttgcagtgactgaaaatgtagtcaatctctatcagaactatagtgagctcatcccagggaccacagttggggttctgtccatggattccagcaatgtcctccagctcattgttgatgcttatgggaaaatccgttctaaagtagagctggaagtgcgtgacctccctgaagagttgtctctatccttcaatgccacctgcctcaacaatgaggtcatccctggcctcaagtcttgtatgggactcaagattggagacacggtgagcttcagcattgaggccaaggtgcgaggctgtccccaggagaaggagaagtcctttaccataaagcccgtgggcttcaaggacagcctgatcgtccaggtcacctttgattgtgactgtgcctgccaggcccaagctgaacctaatagccatcgctgcaacaatggcaatgggacctttgagtgtggggtatgccgttgtgggcctggctggctgggatcccagtgtgagtgctcagaggaggactatcgcccttcccagcaggacgaatgcagcccccgggagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcctggtggtcctgctctcagtgatgggggccattctgctcattggccttgccgccctgctcatctggaaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaatgataagcagtcatcctcagatcattatcagcctgtgccacgattgcaggagtccctgccatcatgtttacagaggacagtatttgtggggagggatttggggctcagagtggggtaggttgggagaatgtcagtatgtggaagtgtgggtctgtgtgtgtgtatgtgggggtctgtgtgtttatgtgtgtgtgttgtgtgtgggagtgtgtaatttaaaattgtgatgtgtcctgataagctgagctccttagcctttgtcccagaatgcctcctgcagggattcttcctgcttagcttgagggtgactatggagctgagcaggtgttcttcattacctcagtgagaagccagctttcctcatcaggccattgtccctgaagagaagggcagggctgaggcctctcattccagaggaagggacaccaagccttggctctaccctgagttcataaatttatggttctcaggcctgactctcagcagctatggtaggaactgctgggcttggcagcccgggtcatctgtacctctgcctcctttcccctccctcaggccgaaggaggagtcagggagagctgaactattagagctgcctgtgccttttgccatcccctcaacccagctatggttctctcgcaagggaagtccttgcaagctaattctttgacctgttgggagtgaggatgtctgggccactcaggggtcattcatggcctgggggatgtaccagcatctcccagttcataatcacaacccttcagatttgccttattggcagctctactctggaggtttgtttagaagaagtgtgtcacccttaggccagcaccatctctttacctcctaattccacaccctcactgctgtagacatttgctatgagctggggatgtctctcatgaccaaatgcttttcctcaaagggagagagtgctattgtagagccagaggtctggccctatgcttccggcctcctgtccctcatccatagcacctccacatacctggccctgtgccttggtgtgctgtatccatccatggggctgattgtatttaccttctacctcttggctgccttgtgaaggaattattcccatgagttggctgggaataagtgccaggatggaatgatgggtcagttgtatcagcacgtgtggcctgttcttctatgggttggacaacctcattttaactcagtctttaatctgagaggccacagtgcaattttattttatttttctcatgatgaggttttcttaacttaaaagaacatgtatataaacatgcttgcattatatttgtaaatttatgtgatggcaaagaaggagagcataggaaaccacacagacttgggcagggtacagacactcccacttggcatcattcacagcaagtcactggccagtggctggatctgtgaggggctctctcatgatagaaggctatggggatagatgtgtggacacattggacctttcctgaggaagagggactgttcttttgtcccagaaaagcagtggctccattggtgttgacatacatccaacattaaaagccacccccaaatgcccaagaaaaaaagaaagacttatcaacatttgttccatgagcagaaaactggagctctggcctcagtgttacagctaaataatctttaattaaggcaagtcactttcttcttcttaaagctgttttctagtttgagaaatgatgggattttagcagccagtcttgaaggtctctttcagtatcaacattctaagatgctgggacttactgtgtcatcaaatgtgcggttaagattctctgggatattgatactgtttgtgtttttagttgggagatctgagagacctggctttggcaagagcagatgtcattccatatcacctttctcaatgaaagtctcattctatcctctctccaaacccgttttccaacatttgttaatagttacgtctctcctgatgtagcacttaagcttcatttagttattatttctttcttcactttgcacacatttgcatccacatattagggaagaggaatccataagtagctgaaatatctattctgtattattgtgttaacattgagaataagccttggaattagatatggggcaatgactgagccctgtctcacccatggattactccttactgtagggaatggcagtatggtagagggataaatagggggcggggagggatagtcatggatccaagaagtccttagaaatagtggcagggaacaggtgtggaagctcatgcctgtaattataaccttcagctactaagacaggtgtggtggctcacgcctgtgattataatcttcagttactaagacagagtccatgagagtgttaatgggacattttctttagataagatgttttatatgaagaaactgtatcaaagggggaagaaaatgtatttaacaggtgaatcaaatcaggaatcttgtctgagctactggaatgaagttcacaggtcttgaagacca

SEQ ID NO: 56 - DNA, 1121 bp, Homo sapiens
gttagagaagggggacacaaatgtcttcaggcttaggtccctgggggctcccagtcctgcccctgttcctctactcacatcccagctcctcccagtttcttttcggacctcctcctctccccttccttctctattccaggctgggttgggctctaagcaaggggagggattagagcctccttcctctctgcccctccccatgggtctctagggggctggtgcaggcagcagcagaggcactctgggcagctgggtgagggcccatctgggcaaggcccccagcgcctgccttctctcccggggccctgtgggcaagcctcctgcttcactttcaggtttctcgaagtgccttcttgctcctgtctgtttccccatcctgccagatttctgtttctcttgctgggcttttggcagtagggggctgtgttggtgggccctacgaagatgctcagtgctcgagatcgccgggaccggcaccctgaggagggggtagttgcagagctccagggcttcgcggtggacaaggccttcctcacctcccacaagggcatcctgctggaaaccgagctggccctgaccctcatcatcttcatctgcttcacggcctccatctctgcctacatggccgcggcgctactggagttcttcatcacacttgccttcctcttcctctatgccacccagtactaccagcgcttcgaccgaattaactggccctgtctggtttttggcatcatcctggtttccatctttgcctatgatgccttcaagatctaccggactgagatggcacccggggccagccagggggaccagcagtgactctggggctacctggctcctaggcccagccagccagagaggacagtggagcccagacacgtctccttgggattcactagcccccagcccgccaaaccccaccccagccctacacagcagtctggcctgagacgtcactggggacttatctgtggagcctggtgctccaggatgtggcttctcatgaagctctggccagaggaggggaacttattgggggagggggggtggaggggaggaatctggacctctaagtcattcccaaattaaaatattcaaattctaaaaaaaaaaaaaaa

SEQ ID NO: 57 - DNA, 863 bp, Homo sapiens
agggccacccaggtgagcctctcactcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaaaaaaaaaaaaaaa

SEQ ID NO: 58 - DNA, 2595 bp, Homo sapiens
agaacaagaaggtatctggtctacaagaactcgaggcctcactgaaacggaaagcaaatacaaagaaactttattttaaaaacatgtcttggtctcccaagaagagggcaattggattgctcagccagaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtattgtggtctgactgacagcttgggtaacttgaagaaccttacaaagctcataatggataacataaagatgaatgaagaagatgctataa

SEQ ID NO: 59 - DNA, 1307 bp, Homo sapiens
acttatctgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaacattggactgtactttgcatttttttctttaaaaatttctattctaacacaacttggttgatttttcctggtctactttatggttattagacatactcatgggtattattagatttcataatggtcaatgataataggaattacatggagcccaacagagaatatttgctcaatacatttttgttaatatatttaggaacttaatggagtctctcagtgtcttagtcctaggatgtcttatttaaaatactccctgaaagtttattctgatgtttattttagccatcaaacactaaaataataaattggtgaatatgaatcttataaactgtggttagctggtttaaagtgaatatatttgccactagtagaacaaaaatagatgatgaaaatgaattaacatatctacatagttataattctatcattagaatgagccttataaataagtacaatataggacttcaaccttactagactcctaattctaaattctacttttttcatcaacagaactttcattcattttttaaaccctaaaacttatacccacactattcttacaaaaatattcacatgaaataaaaatttgctattga

SEQ ID NO: 60 - DNA, 1356 bp, Homo sapiens
tcaggcgaatgctgcatcagtgcccaggcaagcccaggagttgacatttctctgcccagccatgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagctaagctgctcatcacactttaaactcatgaggaccatccctaggggttctgtgcatccatccagccagctcatgccctaggatccttaggatatctgagcaaccagggactttaagatctaatccaatgtcctaactttactagggaaagtgacgctcagacatgactgagatgtcttggggaagacctccctgcacccaactcccccactggttcttctaccattacacactgggctaaataaaccctaataatgatgtgcaaactcttaatggctgaatgggaaaggaaactgcccaagtttgactaattgcttggcctgtgaatggaaaagactctggtctaaaaaaaaaaaaaaaaaa

SEQ ID NO: 61 - DNA, 878 bp, Homo sapiens
atcttagtttccgcaccgcagttcctcggtgtccacttcaggcttccggactggaaggacagccgggaataaaacgtgccggcgaggctcaggagtcattggccacagagacccagcccgagtttcccatcgcactgagcactgagatcctgctggaagctctgccgcagcatgagctccgcagccgggttctgcgcctcacgccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaacttctgatatttgtgttatccttatgattttaaataaacaaaataaatcaagttgtagtatagtcaaaatacttcttaataatagtgcaaaaattgtgttgacacataacaatttcatggaagaaaaaaattccggtattttaagcaaaaagtattttgaaggaaggtgtgaatactggttatgcttggtgttacatgttggctgatacatattcatgcatttacatgattgcagtactttatagctacatatttaccttgaccattattattacctttgccaataaatatcagtaacacagaaaaaaaaaaaaaaaaaaaaaaa

SEQ ID NO: 62 - DNA, 982 bp, Homo sapiens
gtccatgtatctctgagcagctatgaagaagcttcctggaaaacaataagcaaaggaaaacaaatgtgtcccatctcacatggttctaccctactaaagacaggaagatcataaactgacagatactgaaattgtaaagttggaaactacattttgcaaagtcattgaactctgagctcagttgcagtactcgggaagccatgcaggatgaagatggatacatcaccttaaatattaaaactcggaaaccagctctcatctccgttggctctgcatcctcctcctggtggcgtgtgatggctttgattctgctgatcctgtgcgtggggatggttgtcgggctggtggctctggggatttggtctgtcatgcagcgcaattacctacaaggtgagaatgaaaatcgcacaggaactctgcaacaattagcaaagcgcttctgtcaatatgtggtaaaacaatcagaactaaagggcactttcaaaggtcataaatgcagcccctgtgacacaaactggagatattatggagatagctgctatgggttcttcaggcacaacttaacatgggaagagagtaagcagtactgcactgacatgaatgctactctcctgaagattgacaaccggaacattgtggagtacatcaaagccaggactcatttaattcgttgggtcggattatctcgccagaagtcgaatgaggtctggaagtgggaggatggctcggttatctcagaaaatatgtttgagtttttggaagatggaaaaggaaatatgaattgtgcttattttcataatgggaaaatgcaccctaccttctgtgagaacaaacattatttaatgtgtgagaggaaggctggcatgaccaaggtggaccaactaccttaatgcaaagaggtggacaggataacacagataagggctttattgtacaataaaagatatgtatgaatgcatcagtagctgaaaaaaaaaaaaaa

SEQ ID NO: 63 - DNA, 762 bp, Homo sapiens
caggggctggagggcaggggaggggatgatgtcattcctgctcggcgcaatcctgaccctgctctgggcgcccacggctcaggctgaggttctgctgcagcctgacttcaatgctgaaaagttctcaggcctctggtacgtggtctccatggcatctgactgcagggtcttcctgggcaagaaggaccacctgtccatgtccaccagggccatcaggcccacagaggagggcggcctccacgtccacatggagttcccgggggcggacggctgtaaccaggtggatgccgagtacctgaaggtgggctccgagggacacttcagagtcccggccttgggctacctggacgtgcgcatcgtggacacagactacagctccttcgccgtcctttacatctacaaggagctggagggggccctcagcaccatggtgcagctctacagccggacccaggatgtgagtccccaggctctgaagtccttccaggacttctacccgaccctggggctccccaaggacatgatggtcatgctgccccagtcagatgcatgcaaccctgagagcaaggaggcgccctgacacctccggagccccacccccgcccttcccaggtggagccaaagcagcaggcgcctttgcccctggagtcaagacccacagccctcggggaccacctggagtctctccatcctccaccccccgcctgtgggatgccttgtgggacgtctctttctattcaataaacagatgctgcagcctca

SEQ ID NO: 64 - DNA, 1182 bp, Homo sapiens
gaccactcagacaccgtgtcctcttgcctgggagaggggaagcagatctgaggacatctctgtgccaggccagaaaccgcccacctgcagttccttctccgggatggacgtggggcccagctccctgccccaccttgggctgaagctgctgctgctcctgctgctgctgcccctcaggggccaagccaacacaggctgctacgggatcccagggatgcccggcctgcccggggcaccagggaaggatgggtacgacggactgccggggcccaagggggagccaggaatcccagccattcccgggatccgaggacccaaagggcagaagggagaacccggcttacccggccatcctgggaaaaatggccccatgggaccccctgggatgccaggggtgcccggccccatgggcatccctggagagccaggtgaggagggcagatacaagcagaaattccagtcagtgttcacggtcactcggcagacccaccagccccctgcacccaacagcctgatcagattcaacgcggtcctcaccaacccgcagggagattatgacacgagcactggcaagttcacctgcaaagtccccggcctctactactttgtctaccacgcgtcgcatacagccaacctgtgcgtgctgctgtaccgcagcggcgtcaaagtggtcaccttctgtggccacacgtccaaaaccaatcaggtcaactcgggcggtgtgctgctgaggttgcaggtgggcgaggaggtgtggctggctgtcaatgactactacgacatggtgggcatccagggctctgacagcgtcttctccggcttcctgctcttccccgactagggcgggcagatgcgctcgagccccacgggccttccacctccctcagcttcctgcatggacccaccttactggccagtctgcatccttgcctagaccattctccccaccagatggacttctcctccagggagcccaccctgacccacccccactgcaccccctccccatgggttctctccttcctctgaacttctttaggagtcactgcttgtgtggttcctgggacacttaaccaatgccttctggtactgccattcttttttttttttttttcaagtattggaaggggtggggagatatataaataaatcatgaaatcaatacataaaaaaaaaaaaaaa

SEQ ID NO: 65 - DNA, 1044 bp, Homo sapiens
gcccttcccgcctctggggaagggaacttccgcttcggaccgagggcagtaggctctcggctcctggtcccactgctgctcagcccagtggcctcacaggacaccagcttcccaggaggcgtctgacacagtatgatgatgaagatcccatggggcagcatcccagtactgatgttgctcctgctcctgggcctaatcgatatctcccaggcccagctcagctgcaccgggcccccagccatccctggcatcccgggtatccctgggacacctggccccgatggccaacctgggaccccagggataaaaggagagaaagggcttccagggctggctggagaccatggtgagttcggagagaagggagacccagggattcctgggaatccaggaaaagtcggccccaagggccccatgggccctaaaggtggcccaggggcccctggagccccaggccccaaaggtgaatcgggagactacaaggccacccagaaaatcgccttctctgccacaagaaccatcaacgtccccctgcgccgggaccagaccatccgcttcgaccacgtgatcaccaacatgaacaacaattatgagccccgcagtggcaagttcacctgcaaggtgcccggtctctactacttcacctaccacgccagctctcgagggaacctgtgcgtgaacctcatgcgtggccgggagcgtgcacagaaggtggtcaccttctgtgactatgcctacaacaccttccaggtcaccaccggtggcatggtcctcaagctggagcagggggagaacgtcttcctgcaggccaccgacaagaactcactactgggcatggagggtgccaacagcatcttttccgggttcctgctctttccagatatggaggcctgacctgtgggctgcttcacatccaccccggctccccctgccagcaacgctcactctacccccaacaccaccccttgcccaaccaatgcacacagtagggcttggtgaatgctgctgagtgaatgagtaaataaactcttcaaggccaaggga

SEQ ID NO: 66 - DNA, 2047 bp, Homo sapiens
gaacaaacgggatattcagcagtggcctgtggctgccagagcagctcctcaggggaaactaagcgtcgagtcagacggcaccataatcgcctttaaaagtgcctccgccctgccggccgcgtatcccccggctacctgggccgccccgcggcggtgcgcgcgtgagagggagcgcgcgggcagccgagcgccggtgtgagccagcgctgctgccagtgtgagcggcggtgtgagcgcggtgggtgcggaggggcgtgtgtgccggcgcgcgcgccgtggggtgcaaaccccgagcgtctacgctgccatgaggggcgcgaacgcctgggcgccactctgcctgctgctggctgccgccacccagctctcgcggcagcagtccccagagagacctgttttcacatgtggtggcattcttactggagagtctggatttattggcagtgaaggttttcctggagtgtaccctccaaatagcaaatgtacttggaaaatcacagttcccgaaggaaaagtagtcgttctcaatttccgattcatagacctcgagagtgacaacctgtgccgctatgactttgtggatgtgtacaatggccatgccaatggccagcgcattggccgcttctgtggcactttccggcctggagcccttgtgtccagtggcaacaagatgatggtgcagatgatttctgatgccaacacagctggcaatggcttcatggccatgttctccgctgctgaaccaaacgaaagaggggatcagtattgtggaggactccttgacagaccttccggctcttttaaaacccccaactggccagaccgggattaccctgcaggagtcacttgtgtgtggcacattgtagccccaaagaatcagcttatagaattaaagtttgagaagtttgatgtggagcgagataactactgccgatatgattatgtggctgtgtttaatggcggggaagtcaacgatgctagaagaattggaaagtattgtggtgatagtccacctgcgccaattgtgtctgagagaaatgaacttcttattcagtttttatcagacttaagtttaactgcagatgggtttattggtcactacatattcaggccaaaaaaactgcctacaactacagaacagcctgtcaccaccacattccctgtaaccacgggtttaaaacccaccgtggccttgtgtcaacaaaagtgtagacggacggggactctggagggcaattattgttcaagtgactttgtattagccggcactgttatcacaaccatcactcgcgatgggagtttgcacgccacagtctcgatcatcaacatctacaaagagggaaatttggcgattcagcaggcgggcaagaacatgagtgccaggctgactgtcgtctgcaagcagtgccctctcctcagaagaggtctaaattacattattatgggccaagtaggtgaagatgggcgaggcaaaatcatgccaaacagctttatcatgatgttcaagaccaagaatcagaagctcctggatgccttaaaaaataagcaatgttaacagtgaactgtgtccatttaagctgtattctgccattgcctttgaaagatctatgttctctcagtagaaaaaaaaatacttataaaattacatattctgaaagaggattccgaaagatgggactggttgactcttcacatgatggaggtatgaggcctccgagatagctgagggaagttctttgcctgctgtcagaggagcagctatctgattggaaacctgccgacttagtgcggtgataggaagctaaaagtgtcaagcgttgacagcttggaagcgtttatttatacatctctgtaaaaggatattttagaattgagttgtgtgaagatgtcaaaaaaagattttagaagtgcaatatttatagtgttatttgtttcaccttcaagcctttgccctgaggtgttacaatcttgtcttgcgttttctaaatcaatgcttaataaaatatttttaaaggattacagaacaaccaaaaaaaaaaaaaaaaaa

SEQ ID NO: 67 - DNA, 1098 bp, Homo sapiens
gccactcctgctgggcagcccacagggtccctgggcggagggcaggagcatccagttggagttgacaacaggaggcagaggcatcatggagggtccccggggatggctggtgctctgtgtgctggccatatcgctggcctctatggtgaccgaggacttgtgccgagcaccagacgggaagaaaggggaggcaggaagacctggcagacgggggcggccaggcctcaagggggagcaaggggagccgggggcccctggcatccggacaggcatccaaggccttaaaggagaccagggggaacctgggccctctggaaaccccggcaaggtgggctacccagggcccagcggccccctcggagcccgtggcatcccgggaattaaaggcaccaagggcagcccaggaaacatcaaggaccagccgaggccagccttctccgccattcggcggaaccccccaatggggggcaacgtggtcatcttcgacacggtcatcaccaaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctgagccagggaaggaccccctcccccacccacctctctggcttccatgctccgcctgtaaaatgggggcgctattgcttcagctgctgaagggagggggctggctctgagagccccaggactggctgccccgtgacacatgctctaagaagctcgtttcttagacctcttcctggaataaacatctgtgtctgtgtctgctgaacatgagcttcagttgctactcggagcattgagagggaggcctaagaataataacaatccagtgcttaagagtca

SEQ ID NO: 68 - DNA, 1687 bp, Homo sapiens
cgatcgaggctgcagcgcggccgccgggcgcagcatgactgccgtcggcgtgcaggcccagaggcctttgggccaaaggcagccccgccggtccttctttgaatccttcatccggaccctcatcatcacgtgtgtggccctggctgtggtcctgtcctcggtctccatttgtgatgggcactggctcctggctgaggaccgcctcttcgggctctggcacttctgcaccaccaccaaccagacgatctgcttcagagacctgggccaggcccatgtgcccgggctggccgtgggcatgggcctggtacgcagcgtgggcgccttggccgtggtggccgccatttttggcctggagttcctcatggtgtcccagttgtgcgaggacaaacactcacagtgcaagtgggtcatgggttccatcctcctcctggtgtctttcgtcctctcctccggcgggctcctgggttttgtgatcctcctcaggaaccaagtcacactcatcggcttcaccctaatgttttggtgcgaattcactgcctccttcctcctcttcctgaacgccatcagcggccttcacatcaacagcatcacccatccctgggaatgaccgtggaaattttaggccccctccagggacatcagattccacaagaaaatatggtcaaaatgggacttttccagcatgtggcctctggtggggctgggttggacaagggccttgaaacggctgcctgtttgccgataacttgtgggtggtcagccagaaatggcccgggggcctctgcacctggtctgcagggccagaggccaggagggtgcctcagtgccaccaactgcacaggcttagccagatgttgattttagaggaagaaaaaaacattttaaaactccttcttgaattttcttccctggactggaatacagttggaagcacaggggtaactggtacctgagctagctgcacagccaaggatagttcatgcctgtttcattgacacgtgctgggataggggctgcagaatccctggggctcccagggttgttaagaatggatcattcttccagctaagggtccaatcagtgcctaggactttcttccaccagctcaaagggccttcgtatgtatgtccctggcttcagctttggtcatgccaaagaggcagagttcaggattccctcagaatgccctgcacacagtaggtttccaaaccatttgactcggtttgcctccctgcccgttgtttaaaccttacaaaccctggataaccccatcttctagcagctggctgtgcctctgggagctctgcctatcagaaccctaccttaaggtgggtttccttccgagaagagttcttgagcaagctctcccaggagggcccacctgactgctaatacacagccctccccaaggcccgtgtgtgcatgtgtctgtcttttgtgagggttagacagcctcagggcaccatttttaatcccagaacacatttcaaagagcacgtatctagacctgctggactctgcagggggtgagggggaacagcgagagcttgggtaatgattaacacccatgctggggatgcatggaggtgaagggggccaggaaccagtggagatttccatccttgccagcacgtctgtacttctgttcattaaagtgctccctttctagtcctttttctgcccagaa

SEQ ID NO: 69 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 70 - DNA, 3309 bp, Homo sapiens
ggcggagagaataaagcagaaagacaggaagaggaggtggagttctacagttagtgtggttttagtttttcctaagaagtggcgtggtttggggctttatatccgggaggagcatatgtacgcaaatcctggggcgtttgcaaacccggatccggggcgtctggccccatgcccggccgggcgtttgagggctactgccacgcagcgtttctggagcctgccggctggtgccctggtggcctttatctctgtccccctttgtcctctttatctcaggctctccaggaggccggggggcccactccgcctatcgctcccctcggctacgctgccacttcaatgccccgcaggtcgcgagctgctgttctttcgaaggcgtcggagaaccaggggcgtcccgcgccacctctgactcggagcagcgccgagcactgacgctcccgcccttgggcaaggacgccagtgcgcccgcgcgcgtccctctgcgcggcagcccgtcgcgggccctcaaggggaagcccaggccaggatggccccgggtcgcgcggtggccgggctcctgttgctggcggccgccggcctcggaggagtggcggaggggccagggctagccttcagcgaggatgtgctgagcgtgttcggcgcgaatctgagcctgtcggcggcgcagctccagcacttgctggagcagatgggagccgcctcccgcgtgggcgtcccggagcctggccagctgcacttcaaccagtgtttaactgctgaagagatcttttcccttcatggcttttcaaatgctacccaaataaccagctccaaattctctgtcatctgtccagcagtcttacagcaattgaactttcacccatgtgaggatcggcccaagcacaaaacaagaccaagtcattcagaagtttggggatatggattcctgtcagtgacgattattaatctggcatctctcctcggattgattttgactccactgataaagaaatcttatttcccaaagattttgaccttttttgtggggctggctattgggactcttttttcaaatgcaattttccaacttattccagaggcatttggatttgatcccaaagtcgacagttatgttgagaaggcagttgctgtgtttggtggattttacctacttttcttttttgaaagaatgctaaagatgttattaaagacatatggtcagaatggtcatacccactttggaaatgataactttggtcctcaagaaaaaactcatcaacctaaagcattacctgccatcaatggtgtgacatgctatgcaaatcctgctgtcacagaagctaatggacatatccattttgataatgtcagtgtggtatctctacaggatggaaaaaaagagccaagttcatgtacctgtttgaaggggcccaaactgtcagaaatagggacgattgcctggatgataacgctctgcgatgccctccacaatttcatcgatggcctggcgattggggcttcctgcaccttgtctctccttcagggactcagtacttccatagcaatcctatgtgaggagtttccccacgagttaggagactttgtgatcctactcaatgcagggatgagcactcgacaagccttgctattcaacttcctttctgcatgttcctgctatgttgggctagcttttggcattttggtgggcaacaatttcgctccaaatattatatttgcacttgctggaggcatgttcctctatatttctctggcagatatgtttccagagatgaatgatatgctgagagaaaaggtaactggaagaaaaaccgatttcaccttcttcatgattcagaatgctggaatgttaactggattcacagccattctactcattaccttgtatgcaggagaaatcgaattggagtaatagaaaatggaagatggtgttgttaataaaggcatttaatagataaaaacatctccaaaaaggattttgaagctgatcctatttagttaaaaagataattttgctttcaactgtaggtccagaaaactaattattggcatcagtctgtgaaatagtccattatttgttgttaaaaatgcttcaaaaggttttcagtgtcagtctgagatgcctggtatataggagcctttgggaaatacctatttttcagtattccatgcatattagatatcaccatgaagcaagagacatgcattctataatcatgtagacactcagactcaggggaaaatacaagttatatcctgaaagcctttaaaactctatggtaggatcaaagattcaaatggtttcagagaggttttatttcaattaatttgttctagtgctttcaagagcaagtacatcaaaatgtagaaggtaaaatgtatgcaacactaatataaattattccaagtctttaaggagccaaagaaaaaaaagatttctcacagctttttgttctgttttgtatttcaattaggaacttgcagtattattttgaaaaccattctaaaataataggagttaggaaataaataaagttttgctagccctgctaagttcaggcttagaggcttatcgctaagtataaacttcaccagattccacgaaaagctggatagctttttttctgacttatgttgtggttgcacccctcacaaatggcagaacagtatgtaaagctggtaacacctcggtttcagtgcaccatgtgtttgctttgtgaaggtgaagaatatgttggtttagagaaagaaattggatgtaattttatgcaatttacttttaaagacaaacataactatttagcagagaatattttaataaatgcaaaacaacagctggactgctgtacatcaaggacagattaactggaaaacatatgttccttatgtgtgatcgagagccattcagaaaagacttcctttgtgttcagcctatacttttccatatggtataccttgaaaaaaattagcacaccatggttatttttctaccttttataaaagacagagcctgtttactcatttagaagatagagaaaattggtctaaaattgaacatcctagattcacactcccaagtcacttaaggtgatttgatggtgaggaaaatgattgacaaagcccaacaatgatctcaggaattacattttccaacagaccaaaaaatgttttcatgtagcagcaatgcagatttggtgaatatttaatatatattttagtatgtatttcactttatgactgacaattaaaaaatattgtttggccaaatagtaaacacccttttgaaaccatgaaaaaaaaaaaaaaaaa

SEQ ID NO: 71 - DNA, 4098 bp, Homo sapiens
gactccggaagagcccttttctgcagctccttggggactgcacgtctcacttctaagtttgcggcggagagaataaagcagaaagacaggaagaggaggtggagttctacagctctccaggaggccggggggcccactccgcctatcgctcccctcggctacgctgccacttcaatgccccgcaggtcgcgagctgctgttctttcgaaggcgtcggagaaccaggggcgtcccgcgccacctctgactcggagcagcgccgagcactgacgctcccgcccttgggcaaggacgccagtgcgcccgcgcgcgtccctctgcgcggcagcccgtcgcgggccctcaaggggaagcccaggccaggatggccccgggtcgcgcggtggccgggctcctgttgctggcggccgccggcctcggaggagtggcggaggggccagggctagccttcagcgaggatgtgctgagcgtgttcggcgcgaatctgagcctgtcggcggcgcagctccagcacttgctggagcagatgggagccgcctcccgcgtgggcgtcccggagcctggccagctgcacttcaaccagtgtttaactgctgaagagatcttttcccttcatggcttttcaaatgctacccaaataaccagctccaaattctctgtcatctgtccagcagtcttacagcaattgaactttcacccatgtgaggatcggcccaagcacaaaacaagaccaagtcattcagaagtttggggatatggattcctgtcagtgacgattattaatctggcatctctcctcggattgattttgactccactgataaagaaatcttatttcccaaagattttgaccttttttgtggggctggctattgggactcttttttcaaatgcaattttccaacttattccagaggcatttggatttgatcccaaagtcgacagttatgttgagaaggcagttgctgtgtttggtggattttacctacttttcttttttgaaagaatgctaaagatgttattaaagacatatggtcagaatggtcatacccactttggaaatgataactttggtcctcaagaaaaaactcatcaacctaaagcattacctgccatcaatggtgtgacatgctatgcaaatcctgctgtcacagaagctaatggacatatccattttgataatgtcagtgtggtatctctacaggatggaaaaaaagagccaagttcatgtacctgtttgaaggggcccaaactgtcagaaatagggacgattgcctggatgataacgctctgcgatgccctccacaatttcatcgatggcctggcgattggggcttcctgcaccttgtctctccttcagggactcagtacttccatagcaatcctatgtgaggagtttccccacgagttaggagactttgtgatcctactcaatgcagggatgagcactcgacaagccttgctattcaacttcctttctgcatgttcctgctatgttgggctagcttttggcattttggtgggcaacaatttcgctccaaatattatatttgcacttgctggaggcatgttcctctatatttctctggcagatatgtttccagagatgaatgatatgctgagagaaaagataatcaaatgggctactgatgacatcaaatctcaacttcatttactttggatatatacggcaaggtagtaatttcattatctggagtatttcacctccacatctctgcaataccagcaccttcaacagtgattggttagaggttggcatatgacatagttatgaccattgagacgtgatgctgtgtgtgctgtgaagtttctcaaaaggagtaaggaagatacagcctcttctttctagggactttgtttgtaggtgtgacacctggagcagccattttgctactgttttgtttctaattgtgttccccgccgccctccccgctcgccccgaaaaacgacatgtttgagttctaatccccagtatttcagaatgtgaccttatttggaaatagagtcattgcaaatgtaatttgttaagatgagctcatactggagtagagtgggttcctgatctagtatgactggtattcttattaaaaagagaaatttgaatactagctacaccatgtaaagatgaaggcagagatcgtggtgatgcatctacaatgtcaacaattgccagcaaatcatcagaatctgggagagaggtatggaatgaattctctctcgcagccctaagaaggaaccaatcctgtgcacacctgaatcttggacttccagcctccacaactgtgagacaatacatttttgttatttaagctacccagttgtggcactttattactgcagttctagccgactaatacagctaccttgagaggaaatagcatggggatgaaggtgatgtgctgaagaagcacagcagaaagtggaaagatggtccatgaggacatccttagccactgaattgacaagcctcttccctggagtccctcttccttgggattttttttggtgtgtgtaataatcacttttcctaatggtgcaaaccattgtgagttgctctttgactgctagtaattaaagcaaactaaccaagtcagccctccccagtgttcctttttgcaatctaaattctgatgtccaactcctccctgggccactccctgtccccagatgtctctttgtatatctaaagaagaaaaagaaggataaatcctttttctcccaagagatctccctgcttttgtgaaaagcacaaagaagggcattatctaacttttctcatcattagaagtcaagcctggccgtgactcccgttatgacattttgatggccacattttatgagaaaagcccaaacagacagacctgtctgtaaattctttcatcctttgcattctttctttgactgtaccttagagagtgtttgattattgcaggggagatggaaaagaagcaggttctggcctatgctctagttcctcactttctggagttatcttttgggcctggagtctcttggagtgaggaaaggaagccatttttatactttacttatggtagagttaaacttcgtactcttaggaggctacatgcaatctccaagaggaagagagtgaagaggttaagaattatgccatcaaaatgtgctgctctggcatattgactatttgaattacagatattttaaaaacatcagatatgaagaggtcactctgatcttaattctgtgtcttaacagcaggagatgaaattcccacatgggagatggcctccctgtcacagaaggaataccattctccctatcaatgatgagaaattggaactgagagaattctgtatagaccttgttcaagtaattcttctattttaaacctccccacataatttagctgccttttcaaaactactgtcctttgtgcaaccagtatagaagcgattgactctcactgcatccttgggtcttcttttccctatatgggctcttttcccatatgggctcttatgtcctgtaaaacatgtattaaataaatttgtatgcttttctcccgttaacctgtcttacatcaatttaattcttgggcccaggtaggaccctaagaggacagaggtcgagttttgccacctctgcaagagaaaagagagatgcccagaccaccaggcctgggaaatctttgatttcccaggctttgaagtgggcactgtgtctgaacctggccctctgaaacttcctgttcctggacatcccagctgctaatgtgagttgcccttgcactggctcagggctgggacattaaaggaatttcccttttgttgacataacagtaggatggctatagctaacaataatatattatttagtttcaagtagctagaaggaacatatgaatgttcccaacacaaagaaatgacaactatttgagatgatggatatgctaataaccttaatctgattcctatacattatatgcatcacatatacatacattatatgtataacatcactatgtaccccataaatatgtgcaattattgtcaattaaacaaataaatttaaacaaacattaaaaaaaaaaaaaaaaaa

SEQ ID NO: 72 - DNA, 4609 bp, Homo sapiens
agtagcgcaatctcggctcactacaacctctgcctcccgggttcacgtgattctcctgcctcagcctcccgagtagatgagatcacaggcacgtgccgccatgccgggttgacttttgtatttttagtagagacggggtttcaccatgttgcctaggctggtcttgaactcctgacctcaggcgattcacccgcctcggcctcccaaagtgttaggattacaggcgtgagccaccgcgcccggcttgaattgtacacttcaaaaggtggaattttatggtgttgaattatatctttatttttttaacggggggaaaatgacgccgctggagaggagttagcggaactgaaacaatgaaatggtgcgcgagtgtcgcctgtccccgtcgcatccatcccaacgaagtttgggccctggaacggtgcacccagaaggcctgcggggagagacgctggggcatgatctggaagaaagacgtctcaggattcgaagggaatgcagctaaggtggcggcggaggttcgcctaggactggggaggcgtccctaggctcagaagttggcccggccggagcggagatttaaaggttggagcgcagaggctcttaaagaggccgagtcgaattcccactcggcgtccaccttaaagccagctccccggcaccacggatctgacccgggtctgacctacgagaaacatggcaaccagcgccgtccccagtgacaacctccccacatacaagctggtggtggtgggggatgggggtgtgggcaaaagtgccctcaccatccagtttttccagaagatctttgtgcctgactatgaccccaccattgaagactcctacctgaaacatacggagattgacaatcaatgggccatcttggacgttctggacacagctgggcaggaggaattcagcgccatgcgggagcaatacatgcgcacgggggatggcttcctcatcgtctactccgtcactgacaaggccagctttgagcacgtggaccgcttccaccagcttatcctgcgcgtcaaagacagggagtcattcccgatgatcctcgtggccaacaaggtcgatttgatgcacttgaggaagatcaccagggagcaaggaaaagaaatggcgaccaaacacaatattccgtacatagaaaccagtgccaaggacccacctctcaatgtcgacaaagccttccatgacctcgttagagtaattaggcaacagattccggaaaaaagccagaagaagaagaagaaaaccaaatggcggggagaccgggccacaggcacccacaaactgcaatgtgtgatcttgtgacaggcctgaggccctgggcacagtgacggtggcctggccagccctcgggacccctccccacctaactgcactgaaaccatttctaaccacaacccttggcccaaggacttggtacaggaagggagaagggcaggtgggcagggagcagacagggtctggctttgcccagagggcacgggctttcccacctctcaaagagacaaggaagccacctgtaagcagaagcagcatccaagtgcccctggcccccccatgtgttgattcaacccggttcctccccctctctcggtgggtgtgttgtttattgtaactacatagtgttggtttgatgtggaagtgtttatccacatacaaagtacaaaacaagccatgaacaagcttctttcccttaccccccatccacaatgtctgagcttggatgtcttttatagatttttaaattattttagtgattattattttattaaaggggtctgggctcactgcctggtgaagtttcaagtgttcagcagacctctctggtaacatatctggaatattgttgttgttttttaaccgagttttcccatcagtgccaaaactcaactcaatctgaaagtagagtgtctgagaggacagaaggtaatgggaactgtagctggaggcctcaggccatgggtcaaacctgggagggaaagagaccctacacatggcctagaaatgagagaagagagaggtatttacccagaggattttcctatggttggggatgcaaatattagaaaacagattgtattttgctgaggggagtggctgtcatgagcatgtcagttctaaaaggggttttcattatcctggaaatgtataaactaaagtaagctgattggctttgcaaacatgttcatttgtttttcagacagtatgggttaagttctctgccctccccaggggtctgaggaggctctgggtttctcagatctgtctcttgctgcgttttcacatcagctgtgctgcttggtgcctctctgatacgaatacactgacacgtcaaagtaacctaatgtggacaccatccagaaaactccagttcatgctggatcttaaccaaaaatgattcaatactgttatcactaaaacagcaccaagacctgaagccatcttcccttggagtcaactgactaccacctctataagcctagtcaatgagcagaccccttccagtatttgtaaaagtagtactaggttgcctttttggcaatttttattgacctgttgaatcttgactataaaatgatctgagaagtaaggaaggctgggctgatgtgtggctctcatataccttctgcaagggggcagtctccccagctccctgatgatgctcacccccgcccccccacctcaggtgctgctggtgtgagccaaagactggagtttttccagctggggtgggagtggagagacaacaggaacaacgctgcaccaaagaaaaggtcagaataaaaggcagcacagctggtgaccttattttctagatgttacaaatcaggtcactatgcaaactagaatatcctcagcaggtggcctggccactctggagaaagaaacccaaggaaagtgagcacccaactggatgccaagacacccgggttctgaaaatgtgctgtgttcctacctcggcaagatcaccagcactgaggggcccagctggagaatgattctgctacaaaaggagacagttgagacttttgcttgttggaaatcaaacttcttatttgtctaaattgcccctttttctgttcctaaaaggaaggataagagagaacattccaggtgaggcacttcaaagtttccttagaccctatagtgttaagaggtattttaaacactaaaaggacaaagctcttcccaatccttatgcttccctaagtggtatctgcagcagtttgttgtgtgcagtttgatggcagctgcaaactggaggtgaggcggaggaaaggcaggtaggaaggagtaaggatggagatgctcagaatcaagagcatggcggagtaggagaagaagccctgcacacagggcagtgtccacagccagaaaactcctgctgggcaccaaccactacgagcataccccatgcccaccgtggagctgcaactcctcgacagcactgagtttgatagtctcactggaagcagatcagctgatgtagaacagagacctcggccataaaggtgagaagacatagggatttcaaccacacagttgggacagaagggacagtgcatctgttcatccatcctgcacttggcccacgttgaactccatggtgcctgagagagactagttaagggttggtcttctgtatcctctgctgttgagcctctggtaagctttcatctcccatgaactcatttccccataaatgaaatgggtaaataatgccccatttgtagaagtgggccctcatgactgaggtagcttccagataggccagagtagagtgtagagtgtgccccgtgacatccctccatcttctcctccattatcatctagcagggtcagactgggaaacctggttggccacgccacaccatgaccgaggagccaactgggacttctggctgtttgacatcctcatgttcccgttggtcttccggagaatagtgctaccctcacatcccctggagcacagccttcctgaaatgccctcaccccatgcctttgccattgtgtgctctcagatttcttccactgtttgacaccctccttagagggctgctcttttttttccagagataatcctagccatcctctccactcccacggctggggacaatggccacttactacctgtgcactttgccactcgggacacctggatggtttctcttaggactttgcccacctccttctcatggcacttgctgtggaaaatgcctggctggcctcgtggggcctgtctcacttttccaggagacatgacccactaacgtggcaactttaacccaaaggcccctcagacatgttacagcaaatctggagccacagacaggttccctccattggcagcccattgtgtttgaaattccatgtcgggtttacttggaatgaaagatacttgaattattgtgcgcctgtgagcgcccagcttctgtttcatagtcttaacaggtggccattgtcgtgaaacgagtgatgcctgaagatctcagtgatgtttgaaccttctgtgtaactttttattaagtctttgtatctctcgactgattaataaagaagagaaacacgtaaaaaaaaaaaaaaaaaaaaa

SEQ ID NO: 73 - DNA, 4396 bp, Homo sapiens
gcaaggacacacccacagcttacaccattggctgctgtttagctcccttatataacactgtcttggggtttaaacgtaactgaaaatccacaagacagaatagccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatattagttgtgttcaacaattagtgaaacagaatgtgtgtatgcatgtaagaaagagaaatcatttgtatgagtgctatgtagtagagaaaaaatgttagttaactttgtaggaaataaaacattggacttacactaaatgtttaattcattcattttattgtgaaataaaaataaaatccttagctcctccaccaactgaacagaccctcttggccaaggagaccccagaaaccttaaaaactaagtttcccaaccatgacaagatgagagatcattcacacctcattatattccctcccttgctaactgccattggactttttccactgagttaaacagaaacccatggaaaacaaagaacagaagactcactccttggctgacttcacctagctcactccacgtagcgccacagccagactcccctcccctcttgcggtttccacatgacaactgatcagccttccctcctgataagtgaccactgcccacagactggttctggccagtccatggaggctgcacacagggtgcctctatgtcctttgtttcaccttttgatatagaaaggctaattttgctgtattttaatgttaagtctccaccacagagtgaacacagaatgcatgtgacatacatgtttacataccactattgtgtgactgcccctcatgaatattcatagccccccataacctgttaactatgtgtgtctagccaatccaccaaccataaaacttctgtaataccctcccttcctccaagagcctgcttttggttgctgtggtaggctctgcttcccaggctgcaggttgcaggagaggaggctgcagtggctcacgcctgtaatctcagcacttcgatgggacgaggcaggcagatcacctgaacccaggagttcgagagcagccttggcaatggcaaaaccaaccgtctctacaaaaaatgcaaaaacttagctgggtgtggtggcatgcacctgtagcttcagttccagctactcaggaggctgaggtgagtggactgctggagccagggagttcgaggctgcagtgtcgagatcttgccactgcactccattctggatgatagaacgagaccccatctcaaaaaaaaaaaaagttctctccaattgtatatagcttgtgattttatgtcaacactatcaataaatagctttcagtgcaagaaaccaaaaatactgtaataaacaggcacatattcttcccaaacctcatgcagtttacaatctagtgagagacacagatagcagtacagagtcaattaaaggttagttttcttcatgaagatgttttaattttaattcaatgtgaaagggttccaaggagtttatcttgttttatgccattttatttgaagcactacttactaagtcatttgctgatattaatctagttaaatcaagaaatattacatgaaaatgttgctaaatcagagatcatgggtaacaatcacctttgattatgaataatcatattttattgaaaggcaaggcacaacaaataataagaaggaaaaaataaataagcaatgttattgatctttcattctgtatatgttttggggggaatatactagtttcttttagtggctgtaacaaattaccacaaacttggtgacttaaaatttcacagatttactctttcttacagttctggaggtcagaagtctgaaatgggtttcaatgagccaaagtcaaggtattgatgacgctacactcctccggaggctctaggcagatagccttttccagcttccagaggctgcctgaattctttcatccatcttaaaaaccaacagtgtagtagcctcaaatctctctctctgcttccttcttcacatctccttctctcctctgactcttttgcctctttcttctaaggacgcaccaggtccacctgcataatccagaataattgccccatccgcaaatccttaatttaataacatctgcaaagtcccttttgctatgtaaagtagcatgttcacaggttctggagacttggccatggatacgattgcggggggggcattattcttaccacagagcaccccaagaaaatctccaaattttgggcttccaatccattttgcttcaattatttaatatttttactccttccagtagatactgatttcatccattgcccttaagaaggtaggacagagattatggcacatctcacattaaatgctatattttcgttggaaatacattttttgcttcaacttttattttaaattcaagggtacatgtgcaggatgttcaggtttgttacacaggtaaacgtgtgccatggcggtttgctgaacagatcatcccatcaccaacagatcatcccattgagaggtgaagccggctgggcttctgggttgggtggggacttggagaacttttctgtctagctaaagtattgtaaaatggaccagtcaacactctgtaaaatggaccaatcagctctctgtaaaatggaccaatcagcaggatgtgggtggggccaagtaagggaataaaagcaggccacccgagctggcagcggcaacccgctcgggtccccttccatgctgtggaagttttgttctttcgctctttcaataaatcttgctgctgctcaaaaaaaaaaaaaaaaaa

SEQ ID NO: 74 - DNA, 1742 bp, Homo sapiens
tctttgaagcttcaaggctgctgaataatttccttctcccattttgtgcctgcctagctatccagacagagcagctaccctcagctctagctgatactacagacagtacaacagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcgggtctaaaaaaaaaaaaaaaaaa

SEQ ID NO: 75 - DNA, 3950 bp, Homo sapiens
atgtcacatctggtggaccctacatcaggagacttgccagttagagacatagatgctatacctctggtgctaccagcctcaaaaggtaagaatatgaaaactcaaccacccttgagcaggatgaaccgggaggaattggaggacagtttctttcgacttcgcgaagatcacatgttggtgaaggagctttcttggaagcaacaggatgagatcaaaaggctgaggaccaccttgctgcggttgaccgctgctggccgggacctgcgggtcgcggaggaggcggcgccgctctcggagaccgcaaggcgcgggcagaaggcgggatggcggcagcgcctctccatgcaccagcgcccccagatgcaccgactgcaagggcatttccactgcgtcggccctgccagcccccgccgcgcccagcctcgcgtccaagtgggacacagacagctccacacagccggtgcaccggtgccggagaaacccaagagggggccaagggacaggctgagctacacagcccctccatcgtttaaggagcatgcgacaaatgaaaacagaggtgaagtagccagtaaacccagtgaacttgtttctggttctaacagcataatttctttcagcagtgtcataagtatggctaaacccattggtctatgcatgcctaacagtgcccacatcatggccagcaataccatgcaagtggaagagccacccaagtctcctgagaaaatgtggcctaaagatgaaaattttgaacagagaagctcattggagtgtgctcagaaggctgcagagcttcgagcttccattaaagagaaggtagagctgattcgacttaagaagctcttacatgaaagaaatgcttcattggttatgacaaaagcacaattaacagaagttcaagaggcatacgaaaccttgctccagaagaatcagggaatcctgagtgcagcccatgaggccctcctcaagcaagtgaatgagctcagggcagagctgaaggaagaaagcaagaaggctgtgagcttgaagagccaactggaagatgtgtctatcttgcagatgactctgaaggagtttcaggagagagttgaagatttggaaaaagaacgaaaattgctgaatgacaattatgacaaactcttagaaagcatgctggacagcagtgacagctccagtcagccccactggagcaacgagctcatagcggaacagctacagcagcaagtctctcagctgcaggatcagctggatgctgagctggaggacaagagaaaagttttacttgagctgtccagggagaaagcccaaaatgaggatctgaagcttgaagtcaccaacatacttcagaagcataaacaggaagtagagctcctccaaaatgcagccacaatttcccaacctcctgacaggcaatctgaaccagccactcacccagctgtattgcaagagaacactcagatcgagccaagtgaacccaaaaaccaagaagaaaagaaactgtcccaggtgctaaatgagttgcaagtatcacacgcagagaccacattggaactagaaaagaccagggacatgcttattctgcagcgcaaaatcaacgtgtgttatcaggaggaactggaggcaatgatgacaaaagctgacaatgataatagagatcacaaagaaaagctggagaggttgactcgactactagacctcaagaataaccgtatcaagcagctggaaggtattttaagaagccatgaccttccaacatctgaacagctcaaagatgttgcttatggcacccgaccgttgtcgttatgtttggaaacactgccagcccatggagatgaggataaagtggatatttctctgctgcatcagggtgagaatctttttgaactgcacatccaccaggccttcctgacatctgccgccctagctcaggctggagatacccaacctaccactttctgcacctattccttctatgactttgaaacccactgtaccccattatctgtggggccacagcccctctatgacttcacctcccagtatgtgatggagacagattcgcttttcttacactaccttcaagaggcttcagcccggcttgacatacaccaggccatggccagtgaacacagcactcttgctgcaggatggatttgctttgacagggtgctagagactgtggagaaagtccatggcttggccacactgattggagctggtggagaagagttcggggttctagagtactggatgaggctgcgtttccccataaaacccagcctacaggcgtgcaataaacgaaagaaagcccaggtctacctgtcaaccgatgtgcttggaggccggaaggcccaggaagaggagttcagatcggagtcttgggaacctcagaacgagctgtggattgaaatcaccaagtgctgtggcctccggagtcgatggctgggaactcaacccagtccatatgctgtgtaccgcttcttcaccttttctgaccatgacactgccatcattccagccagtaacaacccctactttagagaccaggctcgattcccagtgcttgtgacctctgacctggaccattatctgagacgggaggccttgtctatacatgtttttgatgatgaagacttagagcctggctcgtatcttggccgagcccgagtgcctttactgcctcttgcaaaaaatgaatctatcaaaggtgattttaacctcactgaccctgcagagaaacccaacggatctattcaagtgcaactggattggaagtttccctacataccccctgagagcttcctgaaaccagaagctcagactaaggggaaggataccaaggacagttcaaagatctcatctgaagaggaaaaggcttcatttccttcccaggatcagatggcatctcctgaggttcccattgaagctggccagtatcgatctaagagaaaacctcctcatgggggagaaagaaaggagaaggagcaccaggttgtgagctactcaagaagaaaacatggcaaaagaataggtgttcaaggaaagaatagaatggagtatcttagccttaacatcttaaatggaaatacaccagagcaggtgaattacactgagtggaagttctcagagactaacagcttcataggtgatggctttaaaaatcagcacgaggaagaggaaatgacattatcccattcagcactgaaacagaaggaacctctacatcctgtaaatgacaaagaatcctctgaacaaggttctgaagtcagtgaagcacaaactaccgacagtgatgatgtcatagtgccacccatgtctcagaaatatcctaaggcagattcagagaagatgtgcattgaaattgtctccctggccttctacccagaggcagaagtgatgtctgatgagaacataaaacaggtgtatgtggagtacaaattctacgacctacccttgtcggagacagagactccagtgtccctaaggaagcctagggcaggagaagaaatccactttcactttagcaaggtaatagacctggacccacaggagcagcaaggccgaaggcggtttctgttcgacatgctgaatggacaagatcctgatcaaggacatttaaagtttacagtggtaagtgatcctctggatgaagaaaagaaagaatgtgaagaagtgggatatgcatatcttcaactgtggcagatcctggagtcaggaagagatattctagagcaagagctagacattgttagccctgaagatctggctaccccaataggaaggctgaaggtttcccttcaagcagctgctgtcctccatgctatttacaaggagatgactgaagatttgttttcatgaaggaacaagtgctattccaatctaaaagtctctgagggaaccatagtaaaaagtctcttataaagttagcttgctataacatgaaaaaa

SEQ ID NO: 76 - DNA, 1707 bp, Homo sapiens
ggaaggagcaggaggcagcccaggcggagcgggaggagctggcagcggggcgcatgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcggcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaaggtgagttttaatttgtttattgattgttttgtcatcatgtcccaattttctttccatctttactcatatctaccttttgaatcccaaaagaattgtacaatctgttcctctgatcatctctaccagggaatagttgactcttttacagcattattgttttgtagatttcaaagtacttcatgaacattaatcctttgggttataaatataaccttatcaattgtccagaaacacttagcatactcacacaataaaaattatattagcttgccacctgtctgcccatggtgtgatgtatctataatccacttgttcataaaaaatattcgtctgacacctatgatatgctagggaatatgggagacaataggaaagtaagacagacatggtatctgcccttgtgatgccagtaaacttaagccttcatggggctctctgacttcataatttccagaaccagagataggaaatgaggtgaatttgagaaatgtcagactgtgccaaagggggtcacatgcatcaaatttccacatacat

SEQ ID NO: 77 - DNA, 2442 bp, Homo sapiens
ttggacacacctggccggtgcttcagttagatcaaaccattgctgaaactgaagaggacatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgaaaaccatcgatgaaggaatatctcttctcagaaggaaagaataaccaacaccctgaggttgtgtgtggaaggtgatctggctctggacaggcactatctgggttttggggggacgctataggatgtggggaagttaggaactggtgtcttcaggggccacaccaaccttctgaggagctgttgaggtacctccaaggaccggcctttgcacctccatggaaacgaagcaccatcattcccgttgaacgtcacatctttaacccactaactggctaattagcatggccacatctgagccccgaatctgacattagatgagagaacagggctgaagctgtgtcctcatgagggctggatgctctcgttgaccctcacaggagcatctcctcaactctgagtgttaagcgttgagccaccaagctggtggctctgtgtgctctgatccgagctcaggggggtggttttcccatctcaggtgtgttgcagtgtctgctggagacattgaggcaggcactgccaaaacatcaacctgccagctggccttgtgaggagctggaaacacatgttccccttgggggtggtggatgaacaaagagaaagagggtttggaagccagatctatgccacaagaaccccctttacccccatgaccaacatcgcagacacatgtgctggccacctgctgagccccaagtggaacgagacaagcagcccttagcccttcccctctgcagcttccaggctggcgtgcagcatcagcatccctagaaagccatgtgcagccaccagtccattgggcaggcagatgttcctaataaagcttctgttccgtgcttgtccctgtggaagtatcttggttgtgacagagtcaagggtgtgtgcagcattgttggctgttcctgcagtagaatgggggcagcacctcctaagaaggcacctctctgggttgaagggcagtgttccctggggctttaactcctgctagaacagtctcttgaggcacagaaactcctgttcatgcccatacccctggccaaggaagatccctttgtccacaagtaaaaggaaatgctcctccagggagtctcagcttcaccctgaggtgagcatcatcttctgggttaggccttgcctaggcatagccctgcctcaagctatgtgagctcaccagtccctccccaaatgctttccatgagttgcagttttttcctagtctgttttccctccttggagacagggccctgtcggtttattcactgtatgtccttggtgcctggagcctactaaatgctcaataaataatgatcacaggaaaaaaaaaaaaaaaaaa

SEQ ID NO: 78 - DNA, 2082 bp, Homo sapiens
accacacagacacacacctccttgctggagcattcactaggcgaggcgctccatcggactcactagccgcactcatgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaaataaccatgccaagaagggacattgtcaccaagaaccaggatctctggagaaacagttgggctgttgcatcgagtaatgtcactggactcggcctaaggtttcctggaacttccagattcagagaatgcgatttagggaaacggtggcagatgagtgggagactggttgcaaggtgtgaccgcaggaatcctggaggaatagagagtaaagcttctaggcatctgaaacttttgcttcatctctgacgctcgcaggactgaagatgggcaaattgtaggcatttctgctgagcagagttggagccagagatctacttgtgacttgttggccttcttcccacatctgcctcagactggagggggctcagctcctggggtgatatctagcctgcttgtgagctctagcagggataaggagagctgagattggagggaattgtgttgctcctggagggagcccaggcatcattaaacaagccagtaggtcacctggcttccgtggaccaattcatctttcagacaagctttagcagaaatggactcagggaagagactcacacgctttggttaatatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgggcagttaaaacggcttcaggatagaaagctgtttcccacctctgtttgcttttaacattaaaagggaaatgtgcctctgccccacagttagaggggtgcacgttcctcctggttccttcgcttgtgtttctgtacttaccaaaaatctaccatttcaataaattttgataggagacaaaaaaaaaaaaaaaaaa

SEQ ID NO: 79 - DNA, 3136 bp, Homo sapiens
aaaaccgctgcctctgcactttggaatcccatcttgagactcgctaagcgtcccagccgcatccctcccgcagcgacggcggcccgggacccgcgggctgtgaaccatgaacacccgcaatagagtggtgaactccgggctcggcgcctcccctgcctcccgcccgacccgggatccccaggacccttctgggcggcaaggggagctgagccccgtggaagaccagagagagggtttggaggcagcccctaagggcccttcgcgggagagcgtcgtgcacgcgggccagaggcgcacaagtgcatacaccttgatagcaccaaatataaaccggagaaatgagatacaaagaattgcggagcaggagctggccaacctggagaagtggaaggagcagaacagagctaaaccggttcacctggtgcccagacggctaggtggaagccagtcagaaactgaagtcagacagaaacaacaactccagctgatgcaatctaaatacaagcaaaagctaaaaagagaagaatctgtaagaatcaagaaggaagctgaagaagctgaactccaaaaaatgaaggcaattcagagagagaagagcaataaactggaggagaaaaaaagacttcaagaaaaccttagaagagaagcatttagagagcatcagcaatacaaaaccgctgagttcttgagcaaactgaacacagaatcgccagacagaagtgcctgtcaaagtgctgtttgtggcccacaatcctcaacatgggccagaagctgggcttacagagattctctaaaggcagaagaaaacagaaaattgcaaaagatgaaggatgaacaacatcaaaagagtgaattactggaactgaaacggcagcagcaagagcaagaaagagccaaaatccaccagactgaacacaggagggtaaataatgcttttctggaccgactccaaggcaaaagtcaaccaggtggcctcgagcaatctggaggctgttggaatatgaatagcggtaacagctggggtatatgagaaaatattgactcctatctggccttcatcaactgacctcgaaaagcctcatgagatgctttttcttaatgtgattttgttcagcctcactgtttttaccttaatttcaactgcccacacacttgaccgtgcagtcaggagtgactggcttctccttgtcctcatttatgcatgtttggaggagctgattcctgaactcatatttaatctctactgccagggaaatgctacattatttttctaattggaagtataattagagtgatgttggtagggtagaaaaagagggagtcacttgatgctttcaggttaatcagagctatgggtgctacaggcttgtctttctaagtgacatattcttatctaattctcagatcaggttttgaaagctttgggggtctttttagattttaatccctactttctttatggtacaaatatgtacaaaagaaaaaggtcttatattcttttacacaaatttataaataaattttgaactccttctgtataaatgggtcatttttatttttaatgaaaagttattggggttttctctcttgaagggtctcattttaattcccttttccaggccgtatagatcaaatatagtactgtcattactgttggctcttgttttggtcttgacttactaatagtgttaccctgattttcagagggggacagtttatctccagaaaggccaatgtttgtatacacatcagctagacacaaatatagacatcatatgtagtttgtacatgtttcagaaacttgttttttctttgctctgtgtaacctatttcctattgctagttcagttggctttcttattcacttctgtgaccctgaaccagttctcagaccctagagtgtaagagcattgattttctacgctgtgtaatctagctcaatccctctgtcccctccgcctcaccgtcccccagccaccacattgtatagcaaaagcattacattcaatcctagaataaaggtaaatacaacaaatcatctttgcagctggacaactaataatactttgcagcattaagagatcttctgtgttaccagtcactctgttgaaatgaactttccgaatctctttattcaggaaaacatggggttttgaaattcttgggccaagagacataactgaggggttcgcagagctaggcaagggtgcactaggaaagggccacattggtgggtggggggtaacagagaacagatggtgtcaggaagtttctctggagtaaataatgtggatattcttggtttccctctcctccgccagctgaagctgtgttagtgctgttgacactaatataaaatgtttggtccatttgaaatccttgtcattgccttatatgggggaaactcaatcccccagcctgtgttggaaatatcaccaaactgattgtaaatgtgcggctgtagcagacattttagtgtggtggtgtgcagccatttcggccctacacctgccagcctggctaccttacagttgtgttccgatttttgcgtctatgcttggtgtgcctcacttgctgcattttccagcatgcaaccaggagttgacgtaggaaaaagggatgctttcttactttggaagctctcagggaagttggtgtcaatttctcctccactgcctggcctaccctgcactcccaaagattttgtgcagatgggtagttccattttttaaaaattgtgcagatatggaaaattgtgacttacttcatgaccagaactatctagaatatgtgtgggggtataaacatcttgcttaaccaaatatctatgtaggcagaggtaaccaggagagaagcaagacttgctgcctaaaggagcccaccattttacttttcacatttaatctgccacgttgaatcaattggaataaaacctgactcgcaggtgactggacaggaaatcccaaagttccaccatttctatgcttaattttaatgtccccccgcttttttttttgtagaaaataaaaacaagaaaatcgttccaatgtaagatgtttgttatagaaactttaggcaatacaggtgtgtaataaaatgtttaataaacttctaaacacttttgtatttggattta

SEQ ID NO: 80 - DNA, 3902 bp, Homo sapiens
agaacctggtgcctgcctcagccctagctctggggaaatgaaagccaggctggggttcaaatgagggcagtttcccttcctgtgggctgctgatggaacaaccccatgacgagaaggacccagcctccaagcggccacaccctgtgtgtctctttgtcctgccggcactgaggactcatccatctgcacagctggggcccctgggaggagacgccatgatccccaccttcacggctctgctctgcctcgggctgagtctgggccccaggacccacatgcaggcagggcccctccccaaacccaccctctgggctgagccaggctctgtgatcagctgggggaactctgtgaccatctggtgtcaggggaccctggaggctcgggagtaccgtctggataaagaggaaagcccagcaccctgggacagacagaacccactggagcccaagaacaaggccagattctccatcccatccatgacagaggactatgcagggagataccgctgttactatcgcagccctgtaggctggtcacagcccagtgaccccctggagctggtgatgacaggagcctacagtaaacccaccctttcagccctgccgagtcctcttgtgacctcaggaaagagcgtgaccctgctgtgtcagtcacggagcccaatggacacttttcttctgatcaaggagcgggcagcccatcccctactgcatctgagatcagagcacggagctcagcagcaccaggctgaattccccatgagtcctgtgacctcagtgcacggggggacctacaggtgcttcagctcacacggcttctcccactacctgctgtcacaccccagtgaccccctggagctcatagtctcaggatccttggagggtcccaggccctcacccacaaggtccgtctcaacagctgcaggccctgaggaccagcccctcatgcctacagggtcagtcccccacagtggtctgagaaggcactgggaggtactgatcggggtcttggtggtctccatcctgcttctctccctcctcctcttcctcctcctccaacactggcgtcagggaaaacacaggacattggcccagagacaggctgatttccaacgtcctccaggggctgccgagccagagcccaaggacgggggcctacagaggaggtccagcccagctgctgacgtccagggagaaaacttctgtgctgccgtgaagaacacacagcctgaggacggggtggaaatggacactcggcagagcccacacgatgaagacccccaggcagtgacgtatgccaaggtgaaacactccagacctaggagagaaatggcctctcctccctccccactgtctggggaattcctggacacaaaggacagacaggcagaagaggacagacagatggacactgaggctgctgcatctgaagccccccaggatgtgacctacgcccggctgcacagctttaccctcagacagaaggcaactgagcctcctccatcccaggaaggggcctctccagctgagcccagtgtctatgccactctggccatccactaatccaggggggacccagaccccacaagccatggagactcaggaccccagaaggcatggaagctgcctccagtagacatcactgaaccccagccagcccagacccctgacacagaccactagaagattccgggaacgttgggagtcacctgattctgcaaagataaataatatccctgcattatcaaaataaagtagcagacctctcaattcacaatgagttaactgataaaacaaaacagaagtcagacaatgttttaaattgaatgatcatgtaaatattacacatcaaaccaatgacatgggaaaatgggagcttctaatgaggacaaacaaaaaatagagaaaaattaataaagtcaaaatgtttattcttgaaaacattaatgatacatgaatcttggccacaatgagaaaaataaaaatgaaaaaagagcaggcatccatttccatacaggaacaaaataggaggcagcactacagaccctacacacagctttacagaggtgaaagaaaactgtcagcaattctatgctgacataacagaaaatgtagatgagatagatgaaatacgaaaaattacagtttacttaatgaacataaggataaatagaaaaactgaatcatcatacataaacatatataaaatgcattgatcctgtaatcaaaaatgttcccacaaagtaaatgccacttcagcaaggtttgttggtggttttttcaaactcttatgcactcatgaaacacacagacacacacacacacaaacttgcataaattttccctgagaatattttgtatatatttacacaaatacatttgatcagactaggaacaagttgataccaaaacctgaaaaggaaactacagaatgggaaagtcatagaagatctctcacagaaatataaatcccttaacaaatattaacaagtaagattcatgtctctataaaatagacagtatatcatgaccacactggttttttgttatcctttgattttgtttatgaaaagcaaggatagcttaattttcaaaaactcaatcaatgtaattcagtattttaacaaaaggaatgaaaaattatcatctcaatagacaaagcttttgtctgagcaccttttcatatagctgctgaccatttgtatgtcttcttttgagaaatgcctgttcagctactttgcccatgtttcaagtagtttttggtttcttgctgttgctttgttttagttccttacatatttttgcatattaaccctttatcaggtatacagcttgcaactattttctcccatttctgagttgtctcttcattctgtttgcagaagctgtttagaagccacaccttttgtctatttttgcttttgttgcttgtgttttcagggccatatccaaaaaaaccttgcccggaccaacgtcttgaagcttttctcccacccatttttgtatatgggataagggttcaatttcattcttcttcatatgaatatccccaggatgtgtcctatgcccagctgcacagcttaccctcaaacagaaaataatgaagccttcttcctcccaggaaaggggacgttcagctgagccgagtgtgtatactgctctggccatccactagcccagggaggacccagacctccacactccatggagactcagttctcctaggaccatttattcaaaaggactgccctctcttgttcttggaaactttgttgaggatcaattcaccataaatatgtgtgtttccttctttgctttcatccctgttgcactgatcactgtacctgtttctattccagttccatgatgtcttcctggctgtagctttgtaggatatttggggattccatagtgtgatatccccttcttccctttgctcaagattgttttggctatttggggtccttttgtagtcccattcaaattttaggattgtttttctatttctgtggaaaacgaccttggaattttgttaggaattgcattgagtctgcaggtatgaacttttttttaaagttccagggcacatgtacaggacctgcagctttgttacataggtaggcttgtgccatggtggtttgctgcacctatcaacccattacctagttattaagcccagcatgcattagctctttttcctgatgctctccctcccttcatcatccgccctcccactacaagccccagtgtgtgttgttcccctccctgtgtccatgtgttctcattgttatacgaacattttaacaatgttaattcttgcagaccatgaacataagctaccttcccatttatatgcgtcttgttcaatttcattcatcaatgttataaagattttagtgcaga

SEQ ID NO: 81 - DNA, 2269 bp, Homo sapiens
cagtcttgtgacagggagaacccagcctccagtccacactctgcgtgtttttgtgtcctgccaggcaccgtggtctcatccgcctgcacagctgagtccagtgggagctgacgccatgaccctcaccctctcagtcctgatttgcctcgggctgagtgtgggccccaggacctgcgtgcaggcaggcaccctccccaaacccaccctctgggctgagccagcctctgtgatagctcgggggaagcccgtgaccctctggtgtcaggggcccctggagactgaggagtaccgtctggataaggagggactcccatgggcccggaagagacagaacccactggagcctggagccaaggccaagttccacattccatccacggtgtatgacagtgcagggcgataccgctgctactatgagacccctgcaggctggtcagagcccagtgaccccctggagctggtggcgacaggattctatgcagaacccactcttttagccctgccgagtcctgtggtggcctcaggaggaaatgtgaccctccagtgtgatacactggacggacttctcacgtttgttcttgttgaggaagaacagaagctccccaggaccctgtactcacagaagctccccaaagggccatcccaggccctgttccctgtgggtcccgtgacccccagctgcaggtggaggttcagatgctattactattacaggaaaaaccctcaggtgtggtcgaaccccagtgacctcctggagattctggtcccaggcgtgtctaggaagccctccctcctgatcccgcagggctctgtcgtggcccgcggaggcagcctgaccctgcagtgtcgctctgatgtcggctatgacatattcgttctgtacaaggagggggaacatgacctcgtccagggctctggccagcagccccaggctgggctctcccaggccaacttcaccctgggccctgtgagccgctcccacgggggccagtacagatgctacggtgcacacaacctctcccctaggtggtcggcccccagcgaccccctggacatcctgatcgcaggactgatccctgacatacccgccctctcggtgcagccgggccccaaggtggcctcaggagagaacgtgaccctgctgtgtcagtcatggcatcagatagacactttctttttgaccaaggagggggcagcccatcccccgctgtgtctaaagtcaaagtaccagtcttatagacaccaggctgaattctccatgagtcctgtgacctcagcccagggtggaacctaccgatgctacagcgcaatcaggtcctacccctacctgctgtccagccctagttacccccaggagctcgtggtctcaggaccctctggggatcccagcctctcacctacaggctccacccccacacctggccctgaggaccagcccctcacccccacggggttggatccccagagtggtctgggaaggcacctgggggttgtgactggggtctcagtggccttcgtcctgctgctgttcctcctcctcttcctcctcctccgacatcggcatcagagcaaacacaggacatcggcccatttctaccgtcctgcaggggctgcggggccagagcccaaggaccagggcctgcagaagagggccagcccagttgctgacatccaggaggaaattctcaatgctgccgtgaaggacacacagcccaaggacggggtggagatggatgctcgggctgctgcatctgaagccccccaggatgtgacctacgcccagctgcacagcttgaccctcagacgggaggcaactgagcctcctccatcccaggaaagggaacctccagctgaacccagcatctacgcccccctggccatccactagcccacgggggacccagatctcatactcaacagaaggagactcagagactccagaaggcacaggagctgcccccagtggacaccaatgaaccccagccagcctggacccctaacaaagaccaccaggacatcctgggaactctgggactcactagattctgcagtcaaagatgactaatatccttgcatttttgaaatgaagccacagacttctcaataaatcaatgagctgagaaaactgaaacagaaattagagcatggtataaatttggaatgataatgtaaatattacacattaaatgatgaaatcggaaaactacaaatgagcgaatgaattagaaaagaataaaacctacgtaattaatgaccttggcaatgacag

SEQ ID NO: 82 - DNA, 5140 bp, Homo sapiens
agaggccggaggagggagggggggacaccgagcgcggagagcgcggagagcgcggagggaggcgcgcgcgggagcgaacaccctcccggatccagagcccggcggcggcgaagcagcagctgcggccgcgcccttgccagagccggtgcgtccgcctagccccgctccgcctgaggccgtcagggctcccgaggatggaagattcccaggagacatcgccgtcctccaacaactcctcggaggagctcagctctgctctgcacctgtccaagggcatgtcgatcttcctcgacatactgaggagagcagacaaaaatgatgatggaaaattatcctttgaagaattcaaagcatattttgcagatggtgttctcagtggagaagaattacacgagcttttccataccattgatacacataatactaataatcttgacacagaagagctatgtgaatatttttctcagcacttgggcgagtatgagaatgtactagcagcacttgaagacctgaatctttccatcctgaaggcaatgggcaaaacaaagaaagactaccaagaagcctccaatttggaacaattcgtaactagatttttattgaaggaaaccctgaatcagctgcagtctctccagaattccctggaatgtgccatggaaactactgaggagcaaacccgtcaagaaaggcaagggccagccaagccagaagtcctgtcgattcaatggcctggaaaacgatcaagccgccgagtccagagacacaacagcttctccccaaacagccctcagtttaatgtcagcggtccaggcttattagaagaagacaaccagtggatgacccagataaatagactccagaaattaattgatagactggaaaagaaggatctcaaactcgaaccaccagaagaagaaattattgaagggaatactaaatctcacatcatgcttgtgcagcggcagatgtctgtgatagaagaggacctggaagaattccagctcgctctgaaacactacgtggagagtgcttcctcccaaagtggatgcttgcgtatttctatacagaagctttcaaatgaatctcgctacatgatctatgagttctgggagaatagtagtgtatggaatagccaccttcagacaaattatagcaagacattccaaagaagtaatgtggatttcttggaaactccagaactcacatctacaatgctagttcctgcttcgtggtggatcctgaacaactagatgttcctagacattttctttatggttccaagtgcaaaacaggtgttcttatctaaaacgtcaattagaaaattatctgcggttgttaatctactgtatatttttgtttggtatatttactaagtgcactctttcaaaacttattctataactttatcaattcatgtgaattttagctcaattttcaaagttcactaatattctcaatatttaatgctaaatgctttgctacattgtaactcacctaaaaccttttagtgacaaaatcctaatatgtggaaaaaagcatatgcataaaggaataatattgtgaaaatgaatctgttatgataaagaaaaaataaagtggaaacttttagagtattacttcatagggcagattttgtaaactgtcgtatactgtaaagggttaaatcagcgttttgtgatttttaagtaactgtgagtgaagtttattcttcaacaatgtctactccatccccaacccaactcacagccctatgactactatctttgcattagttaaaaagttagtatataggcatcaaacaaccttggctgtaacctatagaatctctatccacgtatcaggttatagactggtttttcaaaagtgaacaatcctgtgataagttggagtaccatttagtaatacagcaacattgtgtcatttattagcatcataattctttgttatgtaagttaaatatatcaagaaagaagagactgtttggaaaaatgtggttcaagttttatgctatatagttttggtatgcgatacagacagctaacttttcttatgaaaaatacatatttgcatgtaaacaatgatttcaaaatacttgaaaaataaaattttaacccaaatgaataactaagaaatataaaacaagcacaaaatcttagggaagtcataaaatagtagtgaaagtattagacagaagacatctgttttcgaatttcaacactagaatgactaaaactatctacctatagaactatctgtagatagtatactatctacactctgctcaacaagctcagaaattaaatatttttaataataaaaatctgttctggttataaaccttgctaatgaaaatacaatacatataaaaatgtatagccatgttattttctagtataaattcctttgaaactataagtctttgaggaaaattataaggtaaaattttcctgtttttccccctttgaaaaactcaggaaaaaaggaagattgaactaataaaattttatttcttaaatataaatttgacctaaaatattttctcaaactaattcatgaaacagcaacttttaccaatacctttgtatactctcagttctcattcagtataaataaaattttaaaatcctttcatagttctattagaaataagtagtaaattttgatatattgtacatacacacgtgtgtgtgtgtgtgtgtgtgtgtgtgtgtgtgtgtgtgtgtatttgtgtgcctctggtcaactctaaggatgacagacactgtgtaacaacacctgggtcaactcttttaatttatatacaaagcaaagaacaacattaatggagatgcacaatgattattcaaacaagctatatatatgtacaaaggcaaacagacacataacagtctctgcagactgattgtatatagtaagaaaagatcaaaagactttaaaacctaaatgacttttgacatacaaactcttcttgagaatgtttgttgtaaatggtttcaaaaatacaaattatagccaatcaaaacattgctttggttggtgcatttaagtatccaactcaaaaagcatatcaaatattttgggtactaggcagtttccaaagtagcatggtagtattacttgttaaaagggttctgttttcattaacagtactaagtggaagggatctgcagattccaaactggaataagctctatcatattctgaaacaagaattagaatgacttgagaacgggcaaataacaaagcaaaccaatataattatatggtcattctgaccccagctcttatacaaattatacatgtatttttgtgtatgtttgtgagagttgtatgtatgtgaatgtgtgtgagtgtgtattcacatacacatatatactggaacctatagtagaaaaggaaactagtagggccaaaaaaaaaaagaaaaagaaaaagaaaaaagaaaaaaaaagaaaaaactgggacctaagtataaatatctcatcctaaagtaaacaataagtttatagttaacgaagatttttttctatttaaaaccccattttcctaaagaacaaagtagtaaaataaaaaaaaatttaaaaaattaaaaaataaaaaaaagaggtcactaaaagaccaagatgggaaacgtaacatggaatacaagcttgaatctgggaaacaagctcaaaaaacagtgaaaaaaaaagtactggtttaggagttcaaaattcagtgaaacaaactttttgccaatagacctagggatctaagaaatagaattagggagagtttacacttacttacctatatatcagcagatttttctggaagaaaccctttttttttttttttttaaacagtaacatccagagtgacaaattgcaggagatttaatgtataaaatttctaggaattaaaagcttaaacccaaaaattgttcatccatatataagaaattatcgaattaaaacttaatgtatgtcaattattttcaaatgtctaaattcctttggaaataaaaatatcagttttactttgaaattgctcaacttctgctattcatatggtctgattagtgacataagtagcagccgtttttcaacttcagtttcattcactaccattgtttccaaattatcaatctctgctagtaattatgaatttaagaccatattattatcaaaaacctagagaccaatcatatatctgaaaagaaatacccattaaaaattctgcctcctgtttattgagaactatgcattgaacattctgaatcattctaagcctctggagagactgtaatgatccattcatgagctggtatgaaatcagtgggaagcagaacaacagaaggaactttaaaagcaacaagcaattatgtaccatatatacactgtagcaaatattttatatttgagagtctttacagcttacatttccattccattattacaagtgatgaaaaacaaaaaattgcaagtagtatgcaaattataaatatacagtcttcccacttcactaaccaaattcctactttccagtgttacttcccaatttatgcaggaaacctcctgcaaagctgaaactgattagaaaattctttatattttaaaatagctctttctcatttttagagaagtcaaatagccaaccatcaaaattaagaataaattgaattgtcacagtccattacagttattgttgctagatccacctcatttgcagatgtccaaacttaaattcatctgttcttaaaatgctacttaaaactttggttgttttcctgtaatataaaagaaaaagttaatttatcaattgattgaatacagtttttactaattagtttatcaaaccaaatactgtgaacgtaccaggtgtttacagatttaaatgcatgttaccatagaaactattaaagtaactagaactgtcaaataacaaaacggctcatgtttttaaaatatatgtaactcattttaaaatatattaaattgtattccaaacctgttcttctgtttctgtggcacctaggtttaaaatatgtattaatgtgtaaatcacaagtaaaatgaattctaatgtacaagtttgttttaaaaagtgtatgtcaagcttttatttacacaataaaatgttattaaagatggaaagtcta

SEQ ID NO: 83 - DNA, 1636 bp, Homo sapiens
gcggcgggcgcggcgcgatgtgcgagcgggcggcgcgcctgtgcagggccggcgcgcacaggctgctccgggagccgccgcagcagggccgggcgctgggcgggctgctgcgctgggtgggcgccaggatgggcgagccccgggagtcgctggcccccgccgcccccgcggaccccggcccagcctcgccgcgcgggggcaccgccgtcatcctggacattttccgccgtgcggacaaaaatgatgatgggaagctgtccttggaggaattccagctcttctttgcagatggcgtccttaatgagaaagaactggaggatctctttcacacgattgactctgacaacaccaaccatgtggacaccaaggagctgtgtgattactttgtggaccacatgggtgactatgaggatgtcctggcctccctggagaccttgaatcactctgtcctgaaggccatgggttataccaagaaggtatatgagggtgggagcaacgtggaccagtttgtgacccgcttcctcctgaaggagacggccaatcagatccagtcgctgctgagctcagtggagagtgcggtggaggccatcgaggaacagaccagccagctccgacagaaccacatcaaacccagccacagcgcggcacagacctggtgtggaagccccactcccgcctctgcccccaaccacaagctcatggctatggaacaaggcaagacccttccatctgccacggaggatgcaaaggaagagggtctggaagcccagatcagccgcttggcagagctgattgggaggctggagagcaaagcactgtggttcgacctgcagcagcgcctgtcagatgaagatggcaccaacatgcacctgcagctggtccggcaggagatggccgtgtgccccgagcaactgagcgagtttctggactctctgcgccagtatctgcgggggaccactggcgtgaggaactgcttccacatcactgccgtgaggctctcagatggcttcacctttgtcatctatgagttctgggagacagaggaggcgtggaagaggcacctgcagagccccctgtgtaaggcgttccggcacgtcaaggtggacacactgagccagcctgaggccctctccaggatcttggtgccagctgcttggtgcacggtgggacgggactgacagcctcccagaggcccgtggaggagcccaccagccccttcttcttgtgaaggaaatcccgtttttttctagacagacactttggtgcagaagcttcttttcaatccatcctccacaagaaggtgtttccctgttgttaagtgaaggaggccgcccctgcccccacctgagaaggcagagcagtgtctgtgctgccaggtcctggtgaagcccaaggttgaagggggcggcttcctggagccagcacccctgcctcctggtcctggcctctcccctacccctcacatggccacgcatgacccacactgaccacaccctgccctcttcggtgacattcttctacctagtaggagtcatgcccctgtagtgcccaacctagccaggtagccaccactgtgcccaggcgccaaataaaccctggttgggaaaaaaaaaaaaaaaaaaaaaaaaaaaaaaaa

SEQ ID NO: 84 - DNA, 16235 bp, Homo sapiens
acatttggctgggacacaaacgagttaggtgagttatagtctaacgtgagtgagttacctgcgtggtggctgcaggctgagctctaaccagataacatgtccacggattcttgacagagagaaataagatcacttagaaagaaaggatcatttctcccttgagtcacaaggagacagaaacagaaaaaaaagcacaaaagctatgctgctccaatcaaaactgaaaatgcttttaatgtctgagcaatcttaagagtattgatttaagtggacagaatgactgcctggctgatctctctgatgagtattgaagtactacttttggcagtacgtcacctgagtttacatattgaacctgaagaaggtagccttgcagggggaacgtggatcacagtcatttttgatggtttggagttgggtgttctttaccccaacaatggctctcaattggagatacacctggtgaacgtgaacatggtggtgcccgcactgcggagtgttccctgtgacgtctttcctgttttcttggatttgcctgtggtgacatgccggaccagatctgtgctgtctgaagcacatgagggtctgtacttcctggaagcatacttcgggggacagctggtaagcagtccaaatccaggaccacgagatagctgtactttcaagttttccaaggcgcagacacccatcgttcaccaagtttatccaccaagtggtgttccaggaaaactaatacatgtatatggctggattatcactggaagattggaaacttttgattttgatgctgagtacattgatagcccagtgatcttggaagctcaaggagacaaatgggttactccttgctctcttataaataggcagatgggaagctgttatcctattcaggaggaccatggtcttgggactctgcagtgccatgtggaaggcgactacatcggctcccagaatgttagcttctcagtatttaacaaaggaaagtcaatggtccacaagaaggcatggctgatcagtgctaaacaggatcttttcctataccagacacactcagaaatattatctgtgtttccagaaactgggagccttgggggaagaacaaacatcacaattacaggagacttttttgacaattctgcccaggttaccattgcaggcattccatgtgatattagacacgtgtctcccaggaagattgagtgcaccactcgggctccaggaaaagatgtgaggctcaccacccctcagccaggcaatcgagggcttctttttgaagttggagatgctgttgagggactggaactgactgaagccaccccagggtacaggtggcagattgtccctaatgccagttctccatttgggttttggtcacaggaaggacaacctttcagagcacggctcagtgggttctttgtggctccagagacaaataattacactttctggattcaggcagatagccaagcttccttgcatttcagttggtcagaggaaccaaggactaaggtgaaagtggcctccatcagcgtcggcactgctgactggtttgactcctgggagcagaatagggatgaagggacctggcagcagaagactcccaagttggagctgttgggtggagccatgtactacctggaagcagagcatcatgggatagccccaagcagggggatgaggattggtgtccagattcacaacacctggctgaatcctgatgtggtcaccacttacctacgggagaagcaccagatccgagtccgagcccagaggcttccagaagtacaggtgctgaatgtatcaggcagaggaaacttcttccttacttgggacaatgtctctagtcagccaatccctgcaaatgccacagcccatctgattcaaacaaccattgaggagttacttgcagtaaaatgcaaactggaacccctttggtctaacatccttctccggcttggatttgaacgaggcccagaagtttccaactctgatggggacctcaccagtgggacggagcccttctgtggcaggttcagcctccgtcagcctcgacaccttgtccttactcccccggctgcccagaagggctatcggctagatcagtatacacacctgtgtcttgcatacaaaggccacatgaacaagatcctgaagatgattgtgtccttcacaatcggctttcaaaacatggtaaagaataccacctgtgactggagtctcacgaggaccagccccgagagctggcagttcgattgcactgacctctgggagacttgtgtgcgttgcttcggggatctccagccccctccggcaaactccccagtgctggttcatcagatcaaccttctccctctggcccaggagacgggcctgttctatgtggatgaaattattattgcagacacaaacgtaacagtttctcaagctgattctggaacggctcgcccagggggcaatctggtggaatcagtctctgtggtgggatcccctccggtctacagtgtcacctcctggctggcggggtgtggcacggagctcccgctcatcactgcacgctctgtgcccactgaaggaacagaagagggatctggactggtcctggtgacgacacagagacgacagcggacaagtccacctctaggaggacactttcgcatccagcttcctaatacagtgatttctgatgtccctgtacaaatttctgctcatcaccttcaccagctcttacagaataatgccgatgacttcacatccaggtacctcaatgccagtgacttcactgtgaaggaggatctatacacttgctacgaacacgtgtggaccttgtcctggtccactcagattggggatttgcccaattttatcagggtctctgatgaaaaccttactggagtgaatcctgctgcagccacgcgtgtggtatatgatggtggagtttttcttggacccatatttggagacatgttggctactgccaaccagcatactcaggtggttgtgcgagtgaatgatgtaccagctcattgcccaggttcctgctctttccagtacctccaagggtcaactccctgtgtccattctgtgtggtactccattgatggtgacatcaacctaatgatttacattaccggaactggtttctctggtgactcccagttcttgcaggttacagtgaacaaaacgagttgcaaagttattttctcaaaccagaccaatgtagtctgtcagacagatttgctacctgttggaatgcatcggatcttgatgttggtgagaccctctggtcttgccatcagtgccactggagaagacctcttcctaaatgtgaaacctagactggatatggtggagccttccagagctgcggatattggagggctctgggccaccatccgaggctctagtttggaaggtgttagcctgatattatttggatcttactcgtgtgccatcaatgtcgctacaagcaattcaagcagaattcagtgcaaagttccacccagggggaaagatggacgcattgtgaatgtgactgtgatcagaggggactattctgcagttcttcccagagcatttacatatgtctcttccttaaatccagttattgtgactctgagcagaaacataagcaatatagcaggcggtgagaccctggtcattggagtggcgaggctgatgaactatacggatttggatgtggaagtccacgtccaggatgccttggctccggttcacacacagtcggcttggggcctggaggtggcactgcccccactgccagctggtctccacagaatttccgtctctatcaatggggtcagcattcactcacaaggggttgatctccacatccagtacctcacagaagttttcagcatcgagccttgctgtgggtccctgctgggagggaccatcctcagcatctcaggaataggcttcagcagggacccagctttggtttgggtacttgtgggcaatcggtcctgtgacattgtgaacttaacggaggcgagcatctggtgtgaaaccctgccagccccccagatacccgatgcgggcgctcccactgttccagctgccgtggaggtctgggctggcaacaggttcttcgcccgtggtccttcaccaagcttggtggggaaaggcttcaccttcatgtatgaagcggcagcaacaccagtagtcactgccatgcaaggagaaatcacaaatagcagcctgagcctgcatgtgggaggaagtaacctctccaactcagtcatccttctggggaacctgaactgtgatgttgagacacagtccttccagggcaacgtgagcctgtctggatgctccatccctcttcacagtctggaggctggcatctatcctctccaagtacgtcagaagcagatgggatttgctaatatgtctgtggtgctccagcaatttgcagtgatgcctcggataatggccatcttcccatcgcagggttcggcatgtggtgggaccatacttactgtgagggggttgcttcttaactctagaaggaggtcagttcgggttgacctctcgggtccttttacttgtgtgattttgagtttgggagaccacaccattctctgccaggttagcctggagggtgaccccttgcctggagcttccttctccctgaacgtcacagtcctggtcaatgggctaaccagcgagtgtcaggggaattgcactcttttcataagggaagaggcaagtcctgtcatggatgccttgtccacaaacaccagtgggtctctgaccactgtgctgattaggggtcagaggttagccaccacagctgatgagccgatggtatttgtggatgatcaacttccttgcaatgtaactttttttaatgcaagccacgttgtgtgccagacaagagacttggccccaggaccccactacctgtcagttttttatacaagaaatgggtatgcttgttctggtaatgtttccagacacttctacattatgccccaagtgtttcattattttcctaagaatttcagcttacatggtggaagcctcttgaccatagagggcacaggcctgagaggacagaacaccacgtcagtctatattgaccagcagacctgcctgacggtgaacatcggtgctgagctcatccggtgcattgttcccacagggaatggctctgttgccctggaaatagaggtagatggactttggtatcacataggagtcattggttataacaaggcctttaccccagaattgatctctatttctcagagcgatgacatcttaacctttgcagtggcccagatctcaggagctgcaaacattgacatttttataggaatgtcaccctgtgtgggtgtctctggtaaccacaccgttcttcagtgcgtggtcccttcccttccggccggggagtaccacgtcagaggctatgactgcatcagagggtgggcctcatctgccctggtgttcacctcaagagttattattacagcagtgacggagaacttcggctgcctgggtggaaggctggtgcatgtgtttggagcgggattttctccagggaatgtctcagctgctgtgtgtggtgctccctgccgagtcctggctaatgctacagtgtctgccttcagctgcttggttctgcccctggatgtgtccttggccttcctgtgtggcctgaagcgtgaggaggacagctgtgaggctgccagacacacctatgtgcagtgtgatttgacagttgccatggcgacagagcaactgcttgaatcgtggccttacctctacatttgcgaggaaagttcccaatgcctctttgtgccagatcattgggcagagtcaatgtttccatcattctcgggcctctttatcagccctaaattggaaagagatgaagttctcatctataatagctcctgtaacattaccatggaaactgaggcagagatggagtgtgagacgcccaatcagccaattaccgtcaagattactgagatacggaaacgctggggccagaacactcagggcaacttttctttacagttctgccggagatggtccaggactcacagctggtttcctgaaaggctgccacaagatggcgacaacgtcacagtggagaatggccaattgcttctgctggacactaacacaagcatcctcaacttactgcacattaaagggggcaagctgattttcatggccccaggacccatcgagctcagggcacacgccatccttgtttctgatggtggagagctccggattggatccgaagacaagcccttccaaggcagagctcagatcacactctacgggagttcctactcaactcccttctttccctatggagtcaagttcctggctgtgaggaatggaactctttctctgcacggttcactaccagaagtaattgtcacctgtcttagagcaactgcccatgccctagacacagtgctggctttagaagatgctgtggactggaaccctggggatgaagttgtcatcatcagtggaacaggtgttaaaggtgccaaaccgatggaagagattgtcactgtggaaactgtgcaggatacagacctctatcttaagtcacctttgagatattctcacaactttacagagaattgggtggctggagagcaccatattttaaaggccactgtggctctgctcagcaggagtattaccatacaaggaaatctcactaatgagagggagaagctgcttgtttcatgccaggaggccaatgctccagaaggtaatctgcagcactgtttgtattccatgagtgagaagatgctaggatccagggatatgggagccagagtgatcgttcagtccttcccagaagagcccagccaggtccagttgaagggagtgcagtttcaagtcttggggcaagccttccataagcatctgagctcactcactctggtgggagctatgagagagtctttcatacagggctgcacagtgaggaactccttcagtagaggcctcagcatgtgcgggaccttgggcctgaaggtggacagtaatgtattctacaatattttaggtcatgcgctgctagttgggacatgcacggagatgagatatatctcctgggaggcaattcatggaaggaaagatgactggtcaggacatggaaatataataagaaacaacgtgatcatccaggtttctggtgccgagggactctccaatcctgaaatgttgacaccatctggcatctatatctgcagtcccaccaatgttatagaggggaacagagtgtgtggtgctggctatggctactttttccatctcatgaccaaccaaacatcacaagctccgcttctttccttcactcagaacattgcacattcttgtaccaggtatggtctctttgtataccctaaatttcagccaccttgggataatgtcactggcaccactctgttccagagcttcacagtttgggaaagtgcaggtggtgcccagatttttagaagtagcaatcttcgcctgaaaaacttcaaagtttattcatgcagagattttggaattgacgtcttggaaagtgatgcaaatacttcagttactgacagcttattacttggtcattttgcccacaagggaagtctgtgtatgtcatctgggattaaaactcctaaaagatgggaactgatggtgtctaacacaacctttgttaattttgatctcatcaactgtgtggccattagaacctgttcagactgttcccaaggacaaggtggatttactgtgaagaccagccagttgaagtttacaaactcttcaaacttagtggcatttccatttcctcatgcagcaattttggaagacttggatgggtctctgtctgggaaaaacagaagtcacattcttgcttctatggaaaccctttcagcttcttgtttggtcaattcaagctttggtcgggttgtccatggcagtgcctgtggaggaggtgttctttttcatcgtatgtctattggtttagcgaatactcctgaagtttcttatgatttaaccatgactgacagcagaaataaaacaaccactgtcaattatgtacgtgatacattgtctaaccctcgtggctggatggctctgctcttggaccaagagacctactcattgcaatctgagaacctttggatcaacagatctctgcagtactcagcaacctttgacaactttgctcctggtaattacctactgctggtgcacacagatttgccgccttaccctgacatcctcctaagatgtgggagtcgagtgggtctgtcttttccatttcttccatcaccaggtcagaaccaaggctgtgactggttcttcaatagccagctgaggcaactcacctatctggtttcaggtgaaggccaagttcaagtcattctccgggtgaaggaaggtatgcccccaactatttcagcttctacctctgcccctgaatcagctttaaaatggtccctccctgaaacatggcaaggtgttgaagaaggctggggaggatacaacaataccattccaggccctggggatgacgttctcattttacccaacagaactgtccttgtggatacagatcttccattcttcaaagggctgtatgtgatggggaccttagacttccctgtggacagaagcaatgttctgagtgtggcatgcatggtcattgcaggcggggagctgaaagttggtactttagaaaatcccttagaaaaggaacaaaagcttctgattctccttagagcctcagagggagtcttttgtgaccgtatgaatggaattcatattgacccaggaacaattggggtttatgggaaagttcatctttacagtgcttatcctaagaactcctggacacatcttggagctgatattgcctcaggaaatgagagaattatagtagaagatgcagtggattggcgcccccatgacaaaatagtccttagctcctcttcttatgagcctcatgaagcagaggtcctcactgtgaaagaagtcaagggccaccatgtgaggatctatgaacggctcaaacaccggcatattggaagtgtacatgtcacggaggatggccgacacattcgtttggctgctgaggttggactgttgacccgaaatatacaaattcagcctgacgtatcatgtagggggagactgtttgtggggtccttcaggaagtccagccgagaagaattttcaggtgtccttcaacttcttaatgtggaaattcagaacttcgggtcaccattgtactcatctgttgaattcagtaatgtgtcagcaggatcctggatcatatcatctactctgcaccagagctgtggcgggggcattcatgcagctgccagtcatggagtacttttaaatgacaatattgtgtttggcacagctggccatggcatagatttagagggtcaggcctatactgtcactaataaccttgtggttctgatgacacagccagcgtggtccaccatttgggtggcgggaatcaaagtgaaccaggtaaaggacatcaacctccatggcaacgttgtggcaggatcagagagacttggctttcacatccgaggccacaagtgctcctcttgtgaactgctttggtctgacaatgtggcgcattcaagtcttcatggccttcatctctataaggaaagtggacttgacaactgtaccagaatctctggcttcttggctttcaagaactttgactatggtgccatgttacatgtagagaacagcgtggagatagagaacattactctggtagacaatactattggtcttttggcagtagtgtatgtattttctgctccacaaaattccgtcaaaaaagtgcagattgtgcttaggaattcagtcattgtggccaccagctcttcttttgactgcattcaggacaaagtgaagccgcactcagccaacttgacatcaacagatagagctccctccaatccaagaggaggtcgaattggtattctgtggcctgtattcacctcagaaccaaatcagtggcctcaggagccatggcacaaagtgaggaatgatcattcaatttcaggaatcatgaaacttcaagatgttaccttttctagttttgtgaagagttgctatagcgatgacctggatgtctgcattctaccaaatgcagagaacagtggaattatgcacccaataacagcagagaggaccaggatgctaaagataaaagataaaaacaagttctactttccttcattacaacccaggaaagatttaggaaaagtagtctgtcctgaattagactgtgcaagtccaagaaaatatctcttcaaggatctggatgggagagccctgggtctgcctccaccagtttctgtatttcctaaaacagaggcagaatggactgcatccttcttcaacgcaggtacatttagagaagaacagaaatgtacataccaatttctgatgcaaggattcatctgcaaacagactgaccaagtggtcctaattcttgatagcgctgatgccatttgggcaattcagaagttatatccagttgtatctgtgactagtggttttgttgatgtctttagcagtgtaaatgccaatattccctgctctacttctgggtcagtgtctactttctattctatcttacccatcaggcaaatcaccaaagtctgcttcatggatcaaactcctcaagttttgcgcttttttctattggggaacaaaagtacctccaagcttctcttggctgtattctaccatgagctccagagcccccacgtcttcttaggggaaagttttattccacccactctggttcagtcagcttccttattgctgaatgaatctattggtgccaactatttcaacatcatggataacctcttgtatgttgtcctacaaggagaggagcccattgaaatacgctcaggtgtttccattcacttggccctcactgtgatggtttcagtcttagaaaaaggctgggaaatagtaatactcgaaagactaactaacttcttacagattggccaaaaccaaatcaggtttattcacgagatgcctggccatgaagagaccttaaaggccattgctgacagtagagcaaaaagaaagcgcaattgccctactgtgacttgcactagtcattatagaagagttggtcaacgtaggcctctcatgatggaaatgaactcacatagggcttcacccccaatgactgtggaaactatctcaaaagtgattgtcattgaaattggtgattcgccaacagtaaggagcactggaatgatttcatccttatcaagtaacaaattacagaatttggctcatcgagtcatcactgctcaacagactggggtactagagaatgttctgaatatgactatcggggccttactagttactcagtcaaagggagtcattggctatggaaatacaagcagttttaaaactgggaacttgatatatattcggccctatgcactttccatcctagtccagccttcagatggagaagtgggaaatgagcttccagtgcagccacaattggtatttttggatgagcagaatcgaagagtagagtccctgggacctccttcagagccatggacaatttcagcttccctggaaggagcatcagactcagtgctaaaagggtgcacccaggcagaaactcaagatggttatgttagcttctacaacttggcagtcttgatctctgggtcaaactggcactttatttttactgtcacttctcctccaggagtcaattttacagctcgatccaagccatttgctgtcttgcctgtgactaggaaggagaagtcgaccatcatcctggctgcttccctgtcctctgtggcctcatggctggctctgagctgtctggtgtgctgttggcttaaaagaagcaaaagcagaaaaacaaaacctgaagagattcctgaatcccagactaataatcaaaatattcatatccacatctcatccaaacgccgagaatcacaagggcccaaaaaagaagacactgtggtgggagaagatatgagaatgaaggtcatgctgggcaaggtgaaccagtgcccccaccagttgatgaatggagtgtccagaaggaaagttagccgccacattgtccgagaggaagaggctgctgtgcctgctcctggtactactggcatcacatcccatgggcacatctgtgctccaggtgctcctgctcagcaggtgtacctgcaagagactgggaactggaaggagggccaagagcagttgctcagataccagctggcaggccaaaatcagctgctgctgctatgcccagacttcagacaagagaggcagcagttgccagggcaaagtcggctgagtaagcaaagtggcagcttggggctttcccaagagaagaaagcctcctgcggggccactgaggcattctgccttcattcagtacacccggaaactattcaggagcaactgtgatcagggaagttgggggcatttggcctgaaaggcagaatgttcccagtatttctggataataagctggggaagtgaggactgtcctgctgggacaactaagaagagagaatgtggactctgaatccctttttcaactttaaaatggaaaacagtcatataaatgcttacagactgaaaaatgtctcatacatattttgcagctatcatgatcctagttcaatgctaggcaaataatggcacttgttaattatttaccagtttttaacttaagcctgattttaacaacttgataatggtgtaatactgacttataccagcaggggttattattaagcattctctggattgaccacccacaatgctttgagcttcttttatagaagggatcatgaaaaggtctggtcagctacagtttaattccacactgttacagaaaagcatttctttatcctgtagtagcatttgaagggacagttcaacaccctgctccctgatctgtcagagcttaccttccaaccacaaggcaacttcctggctttttacaagtggattttatttcatggtttaaattcaatcattattgagaacctacagtagtgtgagcgtatcaaatttgcacttcatgaggatttacacatgaaaattgatattctcatgggctttgctgagagtttagttaaggacaaagatgtaaaaatgccaaatttgggaaaggaaattttgtatgaccactgttctatttttcagtgattctctctgttagtagctgggtcacatttacaactggaaaagaactacattgggagatcagagaaagccaagtggccaagtcctttatcaaaaagctgctttttggagaagatgatgcataaagctgtgatactaaggagaggagagattatgtcatatgcaactgctctaggctgtgcagggggtttaaatggagatcagaacaaaggctcacctggtattgatacatttggtaaggttgttaaaggcaatttcaccaaattcacacatttgtacggggcccatttcttttcaaactgaagaatactttaaacagcctgactgtgtccttggctctggatccagaaggccttatgctccaagcatgtaaagaaaagatcgcaggaatgtggagagcttccaaaaagagattgctctcagattttcctgaaaatcctgaaaaccaggattttccaatctctacaatgttaaggggttgttgtgtagttgcattggaagagagaaaggaaacacactctttcattgctgaattctaaataatgatgccctgaattagttctatgagaagtactggcagacagctgttatgtctgcagggcatcaaggggcaggtgtgtctcaccctactaagaattggcagaaatgttttaacctcttctttaggcatctggtgagtattgcagatcttcaggctgtccctaaaaggaggtaggtagccttctctgtggtcactgtgttggatttggaagaaaatataagtagatgttaaggtcagtgctctccaggagatttcaatccaggctatctgtgttcagggaatgatttgcaagaagttggtttatagacgtttactctggaggcacaagctggctgccatgctgagtaagtcctctttgtagatgatgtattttgcttgggcaccaacagaattaaaaacgaaaaacaaaaaaaaactcgaatgacttaggcagaacatgccttccctagtttaattagtccctgttataaccagttatttatctttttttgtagattctcggtcttgaagtcatttgcctttgcaatctctactttagtatactcattggatcctcaaagagccgtaagctttttggaaaaaaacatttcagaagtgaaggcttttgaggtttataaaatggtagcagtcttagtccttgccctgcctcagtttacctgcacatgctttctttcatttcctcggattctttcaccactttttaagttttttttccacaggtatttttcctgagctctataccatgcatttagctgcctagtgagaatatccagtaggttatcctatagttactatgaagttaacacagcccaaaaacctacttctccttgtgtttttttccaactcaatgaatgacatcactgcactcagaccagaaatctagaagtcagtcatgactcttcattctatttcgcccttcacaaagagttagctgccagctctgttatttctgccccttctttacccttcttccaatttttattatcttcttatttatcctctatgtgtcacaagaccagtctttctgggaggcatgttttatcctatccctctgcgtaggaataaccataattatcttactaattccatcccagaaaagcctgtgatccctgaggccagaattgactctttgggcctaaatagcagctataaacatcttcagtaacttgccaggctctagcatgtggcatggaattttggcatctagctaaaattcctcaacccatagttaagctactcctctttctcctaaaaaaaaaaaaaaatgagagaattagggaaacaccaaaagcaaagagagcagctctatgttcattcatgtctggagatgagccaatttgagcaccactgtaggccaactctaaatttattcagatcgttaacatgggtaacccagcaaatatttcccagcagtgacaatctacaccctttatctaaataaatattgagatgtgcttttttcctgtgttagttcaaagaatctcttcttgcctcccattgctgattttctgtattgatggactcatttctctcaagagattggacaagctaaaattctagagagttcaaaagtgtttgaaagtacattttaagtgttctcaccacaaaaaatgctagtgtgtgaggtgatacatatgttaattagttcaactgatccattccacaatgtatacatatttcaaactatcgagttgtacatgataaatatatacaatttttatttgtcaaataaagaacatctataaatatttatagatttcaaaattgagaaagaagtatatatggagccacggttgttttgaccatttctccatgctttttgtttggggagggggacattttttggggggcagggactgtgttcagggaaagtacctgaccaacagaacttcaagtctcttccagagtgcgagactcagcttatctgatcaaggtgaacttgcccttcaatagacttgcccttgttactgcccagtgaggccaccaaggaccaagaacagttgagctctctcagggccttcccctttcaacaatttagttgaatgactcccttccatctatcagtcttactccaaatatatgaaattactatacattgaaatgcctgcaaactatatgcaaaaatgagtatgaggaaatcaaatgtaaatcatatgcaaatattattcaaccaagcttctataaaattgtacgtgtcaataacagtaaagaattttaattaattctgtcatttaaaattcatgcattccatattttgctttcagaacttatagtctatgccaattttgaaaaaactataagcacaaggaattaaatactattgagtatagaactgttgatttcaacatacactatagcactttgactaattacagatatatttgatagaaaagtcacatttctacaaggtaattttcaaaaagaaataaaacatttctgtaaactt

SEQ ID NO: 85 - DNA, 14135 bp, Homo sapiens
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacgatgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagagcctcgacacccccccaacccccgcaccaagcagacaaagtcaataaaagagctgtctgactgc

SEQ ID NO: 86 - DNA, 60 bp, Artificial Sequence
  Synthetic
gtgtaaagttgttcgccgcgtggaatgtgagtgtgtttgtgtgcatgaaagagaaagact

SEQ ID NO: 87 - DNA, 60 bp, Artificial Sequence
  Synthetic
atttaggcgcccatggtacaaggaatagtcgcgcaagcatcccgctggtgcctcccggga

SEQ ID NO: 88 - DNA, 60 bp, Artificial Sequence
  Synthetic
ctgatttctcacggcgtgtcaccccaccagggcgcaagcctcactattacttgaactttc

SEQ ID NO: 89 - DNA, 60 bp, Artificial Sequence
  Synthetic
ttacataaaatgggtgatatgcgaacagcaaaccaataaactgtctcaatgctgattcat

SEQ ID NO: 90 - DNA, 60 bp, Artificial Sequence
  Synthetic
cttggacaccagattctttcccattctgtccatgaatcatcttccccacacacaatcatt

SEQ ID NO: 91 - DNA, 60 bp, Artificial Sequence
  Synthetic
gaggagaggagcctaatgagaaaatgaccatctaaagcctgcccttcattggtctggttc

SEQ ID NO: 92 - DNA, 60 bp, Artificial Sequence
  Synthetic
atactgcaggcaatcttaacaccacggcaagtatttgtgcatctacacacatctaaacat

SEQ ID NO: 93 - DNA, 60 bp, Artificial Sequence
  Synthetic
gtagcgtgcacttacaccaacccagatggtacagcccaatacacacccaggatggacgct

SEQ ID NO: 94 - DNA, 60 bp, Artificial Sequence
  Synthetic
gtgggaggtcaatcccctttactgtgacacagtgaaacagatctacccgtacaacaacag

SEQ ID NO: 95 - DNA, 60 bp, Artificial Sequence
  Synthetic
tcgtgcgttgccttgctccgtttttcccaaaaagcactggcttcatcaaggccaccgacg

SEQ ID NO: 96 - DNA, 60 bp, Artificial Sequence
  Synthetic
tcccagcactttgggcctaaacaggcagatcgcttggtctcaggagctcgagaccagcct

SEQ ID NO: 97 - DNA, 60 bp, Artificial Sequence
  Synthetic
caaggtcaggatggcatgggaacaggcctagcagggacacaagcctggagtaaggcagga

SEQ ID NO: 98 - DNA, 60 bp, Artificial Sequence
  Synthetic
cctaacctcattccccaaccacttcattcttgaaagctgtggccagcttgttatttataa

SEQ ID NO: 99 - DNA, 60 bp, Artificial Sequence
  Synthetic
catcaactacatagaagcctacatgacaatgaagatacgaaactgagacatcagggtggc

SEQ ID NO: 100 - DNA, 60 bp, Artificial Sequence
  Synthetic
cctgaccacgctttctagctgttgagctgttttccctgacctccctctaatttatcttgt

SEQ ID NO: 101 - DNA, 60 bp, Artificial Sequence
  Synthetic
tgaggctacggcgctgtcatcgatttcttccctgtgaaaacaagagcaaggccgtggagc

SEQ ID NO: 102 - DNA, 60 bp, Artificial Sequence
  Synthetic
atggaagctgggccttcatgtggcagcaagttttcgagcccacctggaaacacattgggg

SEQ ID NO: 103 - DNA, 60 bp, Artificial Sequence
  Synthetic
aggcactcatttgctccttccccagcctccaattagaacaagccacccaccagcctatct

SEQ ID NO: 104 - DNA, 60 bp, Artificial Sequence
  Synthetic
ctgcaagtttctggactagtctcccaacgtttgcctcccaatgttgtccctttccttcgt

SEQ ID NO: 105 - DNA, 60 bp, Artificial Sequence
  Synthetic
ccctctctccccaacctctgccagggcaatctctaacttcaatcccgccttcgacagtga

SEQ ID NO: 106 - DNA, 60 bp, Artificial Sequence
  Synthetic
tggaggtgggctgggccctctcttctcacctttgttttttgttggagtgtttctaataaa

SEQ ID NO: 107 - DNA, 60 bp, Artificial Sequence
  Synthetic
gagttcccggagtgagttgaaccaggtggaccaagtgggctacgtgacctatgacatcct

SEQ ID NO: 108 - DNA, 60 bp, Artificial Sequence
  Synthetic
gttcgacgtgaaggcgcagatggtggatccccggagcgccagcgaggtggaccggatgtt

SEQ ID NO: 109 - DNA, 60 bp, Artificial Sequence
  Synthetic
ccttccttatcgccgacaagtggcccgcgctgccccgcaagctggactcggtctttgagg

SEQ ID NO: 110 - DNA, 60 bp, Artificial Sequence
  Synthetic
ctccaggtccggaggggttgcgggggagctggaaataaacctggagatgatgatgatgat

SEQ ID NO: 111 - DNA, 60 bp, Artificial Sequence
  Synthetic
tcgtgggatgtgcgcgccgagaccacatgtcactgccagtgcgcgggcatggactggacc

SEQ ID NO: 112 - DNA, 60 bp, Artificial Sequence
  Synthetic
gaagaagccatcaatgagaggatccaggaggtcgccggctccctaatatttagggcaata

SEQ ID NO: 113 - DNA, 60 bp, Artificial Sequence
  Synthetic
gacctggctacttgcccccgaggcttcgccgtcaccggctgcacttgtggctccgcctgt

SEQ ID NO: 114 - DNA, 60 bp, Artificial Sequence
  Synthetic
ctctttgggtgatagtcagagagtggtgtttttgttcaggtgggaaggattggaaactct

SEQ ID NO: 115 - DNA, 60 bp, Artificial Sequence
  Synthetic
gctgctattaacaagctagtctgtgatggaccaaatggatgcaagtgtcttgggccagag

SEQ ID NO: 116 - DNA, 60 bp, Artificial Sequence
  Synthetic
tactgcccgagcacgacatggagcttgcgtttgacgttcaattcagcgtggaggatgtcg

SEQ ID NO: 117 - DNA, 60 bp, Artificial Sequence
  Synthetic
agccctacgagtggaatcaggttgatccaaagctggtcatggagcaggccgaccgtgaga

SEQ ID NO: 118 - DNA, 60 bp, Artificial Sequence
  Synthetic
aggaggttgtgtcactgactcaggctgctccttctctagtttcccctctcatctgacctt

SEQ ID NO: 119 - DNA, 60 bp, Artificial Sequence
  Synthetic
cagagatggctccttgggatgaagagggtagagtgggctgctggtgtcgcatcaagattt

SEQ ID NO: 120 - DNA, 60 bp, Artificial Sequence
  Synthetic
ctgtactggctgggctgctggtgctagcactgctggtgctgctcctgtggaagatgggat

SEQ ID NO: 121 - DNA, 60 bp, Artificial Sequence
  Synthetic
ggtagggtgagaagggcaggggtgtcctgatgcaaaggtggggagaagggatcctaatcc

SEQ ID NO: 122 - DNA, 60 bp, Artificial Sequence
  Synthetic
ttatgctgctcctgatataacactttccagcctatagcagaagcacattttcagactgca

SEQ ID NO: 123 - DNA, 60 bp, Artificial Sequence
  Synthetic
ccagtatgtcagttcagtcggaacaaagtttccagtcaagtggtcagctccagaggtgtt

SEQ ID NO: 124 - DNA, 60 bp, Artificial Sequence
  Synthetic
acagcagcaagtcagacgtatgggcatttgggatcctgatgtgggaggtgttcagcctgg

SEQ ID NO: 125 - DNA, 60 bp, Artificial Sequence
  Synthetic
agacaagcattgaagaagaaattaggagtgctgataagaatgaatatagatgctggccag

SEQ ID NO: 126 - DNA, 60 bp, Artificial Sequence
  Synthetic
gataagatgtggtttgaagctgatgggtgccagccctgcattgctgagtcaatcaataaa

SEQ ID NO: 127 - DNA, 60 bp, Artificial Sequence
  Synthetic
gaagaccatagctgagaactaatgcaaggctggccggaagcccttgcctgaaagcaagat

SEQ ID NO: 128 - DNA, 60 bp, Artificial Sequence
  Synthetic
gcagtgcctttgccgttcacgacctggaggaggacacctggtatgcgactgggatcttaa

SEQ ID NO: 129 - DNA, 60 bp, Artificial Sequence
  Synthetic
ggcgaaatgccaattttaaatttactgaccatctgaagtatgtcatgctgcctgtggctg

SEQ ID NO: 130 - DNA, 60 bp, Artificial Sequence
  Synthetic
ggagggggaagagaaatttttatttttgaacccctgtgtcccttttgcataagattaaag

SEQ ID NO: 131 - DNA, 60 bp, Artificial Sequence
  Synthetic
ttccagttctgacacacgtatttatatttggaaagagaccagcaccgagctcggcacctc

SEQ ID NO: 132 - DNA, 60 bp, Artificial Sequence
  Synthetic
ccaactgtgacaagcatggcctgtacaacctcaaacagtgcaagatgtctctgaacgggc

SEQ ID NO: 133 - DNA, 60 bp, Artificial Sequence
  Synthetic
cctcaagtcgggtatgaaggagctggccgtgttccgggagaaggtcactgagcagcaccg

SEQ ID NO: 134 - DNA, 60 bp, Artificial Sequence
  Synthetic
gctacaaggtggtgggcggtgaacgagagaatgtctccatggtggactatgctcacaaca

SEQ ID NO: 135 - DNA, 60 bp, Artificial Sequence
  Synthetic
tgggctctgaacacacacgccagctcctctctgaagcgactctcctgtttggaacggcaa

SEQ ID NO: 136 - DNA, 60 bp, Artificial Sequence
  Synthetic
ttctggatctgaccctcccagtctcatctcctgaccctcccactcccatctccttacctc

SEQ ID NO: 137 - DNA, 60 bp, Artificial Sequence
  Synthetic
cctcagcctctgcaactgcaagaaaggggacccaagaaaccaaagtctgccgcccacctc

SEQ ID NO: 138 - DNA, 60 bp, Artificial Sequence
  Synthetic
gaacaagcagaacagacgctaaaacaggcagcttcaacagatagtctcagggtcttaaat

SEQ ID NO: 139 - DNA, 60 bp, Artificial Sequence
  Synthetic
aatattaatgtctagttgttctatattataaccacatttgcgctctatgcaagcccttgg

SEQ ID NO: 140 - DNA, 60 bp, Artificial Sequence
  Synthetic
cattatagatactctggcattacgcttctataccttttaggtcttccttgcaatactgga

SEQ ID NO: 141 - DNA, 60 bp, Artificial Sequence
  Synthetic
cttgatgtaccagtccaataccatgtagcgctgagtgataaagttaaaatgtgctgtgct

SEQ ID NO: 142 - DNA, 60 bp, Artificial Sequence
  Synthetic
ataattttcctcctaaacaaaaacacattgagtttaagtctctgactcttgcctttccac

SEQ ID NO: 143 - DNA, 60 bp, Artificial Sequence
  Synthetic
tggaaaaacagtgtggatataagctgttcatttatggaagggatgactacgttggggaag

SEQ ID NO: 144 - DNA, 60 bp, Artificial Sequence
  Synthetic
aaagccaaatttatatgccaccgattgcaggacacaagcacagttttaagagttgtatga

SEQ ID NO: 145 - DNA, 60 bp, Artificial Sequence
  Synthetic
accatccttcccatgatgccgctcttctgtcatcccgctcctgctgaaacacctcccagg

SEQ ID NO: 146 - DNA, 60 bp, Artificial Sequence
  Synthetic
ggatctgaagacatagcaaaggacatggaagcctggaaagatgtaaccagtggaaatgct

SEQ ID NO: 147 - DNA, 60 bp, Artificial Sequence
  Synthetic
gctgaggcaggagaatggtgtgaacctgggaggtggagcttgcagtgaaccgagatcgct

SEQ ID NO: 148 - DNA, 60 bp, Artificial Sequence
  Synthetic
gtagtcccatcataagggcagatctgaacattgttagaagttgcacctctaacgtaccat

SEQ ID NO: 149 - DNA, 60 bp, Artificial Sequence
  Synthetic
aattacacattttctactgtcagggagattcgttacataaatatatttacgtatctgggg

SEQ ID NO: 150 - DNA, 60 bp, Artificial Sequence
  Synthetic
tggtacaaggattctcttcttttggataaagacaatgagaaatttctaagtgtgaggggg

SEQ ID NO: 151 - DNA, 60 bp, Artificial Sequence
  Synthetic
tcaaggaagcctcctccacgttctcctggggcattgtgctggccccactttcactggcct

SEQ ID NO: 152 - DNA, 60 bp, Artificial Sequence
  Synthetic
catttgcccatgaaggccagcaatacaacatcactaggagtattgagctacgcatcaaga

SEQ ID NO: 153 - DNA, 60 bp, Artificial Sequence
  Synthetic
ccgaggggccacgccaggaatattcagaaaataatgagaactacattgaagtgccattga

SEQ ID NO: 154 - DNA, 60 bp, Artificial Sequence
  Synthetic
gacattgatttgctcttactacagcttcagtgattgggggaggaaaagtcccaacccaat

SEQ ID NO: 155 - DNA, 60 bp, Artificial Sequence
  Synthetic
gactgaatctctcacctattcttgcagaaagacatactaattaaaccttgtcaaagtagt

SEQ ID NO: 156 - DNA, 60 bp, Artificial Sequence
  Synthetic
attcggcagcaaacttgggctgcagtgcatcggtatgcatgagaaaggcatcatatttaa

SEQ ID NO: 157 - DNA, 60 bp, Artificial Sequence
  Synthetic
gctcagagatactcccaactgatgcagaaaccaaataaagaggtaggtattccaagaatt

SEQ ID NO: 158 - DNA, 60 bp, Artificial Sequence
  Synthetic
atctgacttctaggatttattgttatattacttgcctatctgacttcatacatccctcag

SEQ ID NO: 159 - DNA, 60 bp, Artificial Sequence
  Synthetic
ggcatagtcacctaggggaggaggccgtatgaagacagaggcagagattggagtgacgca

SEQ ID NO: 160 - DNA, 60 bp, Artificial Sequence
  Synthetic
gaggcttattcagttcttacacattccatcttacattagtgattccatcaaagagaagga

SEQ ID NO: 161 - DNA, 60 bp, Artificial Sequence
  Synthetic
gatgggtttttttgttaagaactataggatttatgggaccaagtctagcgagtccagata

SEQ ID NO: 162 - DNA, 60 bp, Artificial Sequence
  Synthetic
ttttttaagcatcctgacaggaaatgttttcttctacatggaaagatagacagcagccaa

SEQ ID NO: 163 - DNA, 60 bp, Artificial Sequence
  Synthetic
ttagaactccttactctgatgtctgtatatgttgcactgaaaaggttaatatttaatgtt

SEQ ID NO: 164 - DNA, 60 bp, Artificial Sequence
  Synthetic
gcccagtcacagaaggaggaatgactcaaatgcccaaaaccaagaacacattgcagaagt

SEQ ID NO: 165 - DNA, 60 bp, Artificial Sequence
  Synthetic
atgaggctctaacactgctcaggagacccctgccctctagttggttctgggctttgatct

SEQ ID NO: 166 - DNA, 60 bp, Artificial Sequence
  Synthetic
tggccaaggttatcggaaatctggagatgcagatactgtgtttccttgctcttcgtccat

SEQ ID NO: 167 - DNA, 60 bp, Artificial Sequence
  Synthetic
cccaggagcccccaggatctcatgaatatgcggcactgaaagtgtagcaagaagacagcc

SEQ ID NO: 168 - DNA, 60 bp, Artificial Sequence
  Synthetic
atgaaaacagacaccagaaccatctttgtcgccatcttcagctgcatctccatccttctc

SEQ ID NO: 169 - DNA, 60 bp, Artificial Sequence
  Synthetic
aggtgaacgactctgggtacaagcaggaacagagctcggcagaaaacgaagctgaattcc

SEQ ID NO: 170 - DNA, 60 bp, Artificial Sequence
  Synthetic
agctccgcgtgagaagtactggctacaattttttatcccattgttggtggtgattctgtt

SEQ ID NO: 171 - DNA, 60 bp, Artificial Sequence
  Synthetic
gccatggttggaggaactgggatgtgtacaaggtgatctattataaggatggtgaagctc

SEQ ID NO: 172 - DNA, 60 bp, Artificial Sequence
  Synthetic
gtcagttccaccaaatggttccacaatggcagcctttcagaagagacaaattcaagtttg

SEQ ID NO: 173 - DNA, 60 bp, Artificial Sequence
  Synthetic
agtggcatgtaatagtaagtgctcaattaacattggttgaataaatgagagaatgaatag

SEQ ID NO: 174 - DNA, 60 bp, Artificial Sequence
  Synthetic
cccatgtcctaaaaactggatatgttacaaaaataactgctaccaattttttgatgagag

SEQ ID NO: 175 - DNA, 60 bp, Artificial Sequence
  Synthetic
atctcaataaaagccaggaacagagaagagattacaccagcggtaacactgccaactgag

SEQ ID NO: 176 - DNA, 60 bp, Artificial Sequence
  Synthetic
tgtttgtcccactattgtattttggaagcacataacttgtttggtttcacaggttcacag

SEQ ID NO: 177 - DNA, 60 bp, Artificial Sequence
  Synthetic
gctgttttaatttctaaaggtaggaccaacacccaggggatcagtgaaggaagagaaggc

SEQ ID NO: 178 - DNA, 60 bp, Artificial Sequence
  Synthetic
tcaacccttggaataacagtctagctgattgttccaccaaagaatccagcctgctgctta

SEQ ID NO: 179 - DNA, 60 bp, Artificial Sequence
  Synthetic
cagaaatcatcaatagaaaaatgcagtgtggacattcaacagagcaggaataaaacaaca

SEQ ID NO: 180 - DNA, 60 bp, Artificial Sequence
  Synthetic
ccctgaaacttagctgtgctgggattattctccttgtcttggttgttactgggttgagtg

SEQ ID NO: 181 - DNA, 60 bp, Artificial Sequence
  Synthetic
agatggatctgccaaaaagaactaacacctgtgagaaataaagtgtatcctgactcttga

SEQ ID NO: 182 - DNA, 60 bp, Artificial Sequence
  Synthetic
gtcatcaacctaacaaacacaaccttctcagcagcatttctcccctgtgatggaaataaa

SEQ ID NO: 183 - DNA, 60 bp, Artificial Sequence
  Synthetic
ctggtgcccagtcggggtggctgagctggtccttaataggttgtttcttggtcttgcttt

SEQ ID NO: 184 - DNA, 60 bp, Artificial Sequence
  Synthetic
gaactgtgactatctatctcccccgacttctaccagggatgccttcacgccaaggctgtt

SEQ ID NO: 185 - DNA, 60 bp, Artificial Sequence
  Synthetic
tggtttgagggggcgggcggtgtgtgtgtgttctggtgggagggatctgagcaagtgcaa

SEQ ID NO: 186 - DNA, 60 bp, Artificial Sequence
  Synthetic
agaagatcactgaagaaacttctgctttaatggctttacaaagctggcaatattacaatc

SEQ ID NO: 187 - DNA, 60 bp, Artificial Sequence
  Synthetic
cctctggaataaaacatacaggagtctgtctctgctatggaatgccccatggggcatctc

SEQ ID NO: 188 - DNA, 60 bp, Artificial Sequence
  Synthetic
ggatatgcctcttcgattgctccgtactctaacatctagctggcttccctgtctattgcc

SEQ ID NO: 189 - DNA, 60 bp, Artificial Sequence
  Synthetic
cgtttacgactgcagggtggagcactggggcttggatgagcctcttctcaagcactggga

SEQ ID NO: 190 - DNA, 60 bp, Artificial Sequence
  Synthetic
aaacccgtgattttggtgctccttgtaactcagccctgcaaagcaaagtcccattgattt

SEQ ID NO: 191 - DNA, 60 bp, Artificial Sequence
  Synthetic
ctccaacctaacctgtgtctgcgaagtcctatggaaacccgagggttgattaaggcagta

SEQ ID NO: 192 - DNA, 60 bp, Artificial Sequence
  Synthetic
attggaacctgccacttggcattagagggtctttcatggggagagaaggagactgaatta

SEQ ID NO: 193 - DNA, 60 bp, Artificial Sequence
  Synthetic
gcctgggatgaatttggtgcctttccatatctcgttctctctccttcccctgcgtttcct

SEQ ID NO: 194 - DNA, 60 bp, Artificial Sequence
  Synthetic
ctagttgctgtggaggaaattcgtgatgttagtaaccttggcatctttatttaccaactt

SEQ ID NO: 195 - DNA, 60 bp, Artificial Sequence
  Synthetic
attaaggtttatgggatactcaagatatttactcatgcatttactctattgcttatgctt

SEQ ID NO: 196 - DNA, 60 bp, Artificial Sequence
  Synthetic
aatgactgcttacctggacttgttgctggggaactgctatctgatgcccctcaatacttc

SEQ ID NO: 197 - DNA, 60 bp, Artificial Sequence
  Synthetic
tgttggtggagcctgcatttacaagtacttcatgcccaagagcaccatttaccgtggaga

SEQ ID NO: 198 - DNA, 60 bp, Artificial Sequence
  Synthetic
tgcctaaccttttggagccttagtctcccagactgaaaaaggaagaggatggtattacat

SEQ ID NO: 199 - DNA, 60 bp, Artificial Sequence
  Synthetic
taaaatcccagctacttgagagactgaggcaggagaatcgcttgaacccaggaggtggag

SEQ ID NO: 200 - DNA, 60 bp, Artificial Sequence
  Synthetic
agcattacccttctgacactctctatgtagcctccctgatcttctttcagctcctctatt

SEQ ID NO: 201 - DNA, 60 bp, Artificial Sequence
  Synthetic
tcagaaatatttcttggaccttccacttctcctccaactccttgaccaccatcctgtatc

SEQ ID NO: 202 - DNA, 60 bp, Artificial Sequence
  Synthetic
ctggatagtcctgtcaccgtggagtggaaggcacagtctgattctgcccggagtaagaca

SEQ ID NO: 203 - DNA, 60 bp, Artificial Sequence
  Synthetic
tgcttgtctgccacgtgacggatttctacccaggcagcattcaagtccgatggttcctga

SEQ ID NO: 204 - DNA, 60 bp, Artificial Sequence
  Synthetic
catttgctgtgtttcgttagcatctggctccaggacagaccttcaacttccaaattggat

SEQ ID NO: 205 - DNA, 60 bp, Artificial Sequence
  Synthetic
taatgggacacagcgcttcctggagagatacatctacaaccgggaggagctcgtgcgctt

SEQ ID NO: 206 - DNA, 60 bp, Artificial Sequence
  Synthetic
taccaagaatctgtggaaatataagctggggcaaatcagtgtaatccttgactttgctcc

SEQ ID NO: 207 - DNA, 60 bp, Artificial Sequence
  Synthetic
atgcagacagacctcaacattcaacaacatccatacagcactgctggaggaagaggaaga

SEQ ID NO: 208 - DNA, 60 bp, Artificial Sequence
  Synthetic
tcctccctcttcatcaacatagtaaaataagtcaaacaaaatgagaacaccaaattttgg

SEQ ID NO: 209 - DNA, 60 bp, Artificial Sequence
  Synthetic
aaacaagcaaagataggtaggacagaaaggaagacagccagatccagtgattgacttggc

SEQ ID NO: 210 - DNA, 60 bp, Artificial Sequence
  Synthetic
tacgtgatagtataaaccaatgtgacttcatgtgatcatatccaggatttttattcgtcg

SEQ ID NO: 211 - DNA, 60 bp, Artificial Sequence
  Synthetic
atctaaaagtgaataatggcacaagaagaaatataagtaataaggacctttgtgcttcga

SEQ ID NO: 212 - DNA, 60 bp, Artificial Sequence
  Synthetic
ttccaggcttttgctactcttcactcagctacaataaacatcctgaatgttttcttaaaa

SEQ ID NO: 213 - DNA, 60 bp, Artificial Sequence
  Synthetic
caaataccaagggaagtgttattggttctctaatgagatgaaaagctggagtgacagtta

SEQ ID NO: 214 - DNA, 60 bp, Artificial Sequence
  Synthetic
gagtttcttatgtgccctggtggacacttgcccaccatcctgtgagtaaaagtgaaataa

SEQ ID NO: 215 - DNA, 60 bp, Artificial Sequence
  Synthetic
gacaccgtgttcagcaccagcctcagaagaggcctcctgctccgtcgggcacacaagttc

SEQ ID NO: 216 - DNA, 60 bp, Artificial Sequence
  Synthetic
ctctgaaaattaagcatctgaagaccgatgatcaggatatctacaaggtatcaatatatg

SEQ ID NO: 217 - DNA, 60 bp, Artificial Sequence
  Synthetic
acacaaccctgacctgtgaggtaatgaatggaactgaccccgaattaaacctgtatcaag

SEQ ID NO: 218 - DNA, 60 bp, Artificial Sequence
  Synthetic
ctgagctacctgagcgtaaggggcgggttcaacatgtcctctttcaagttaaaagaataa

SEQ ID NO: 219 - DNA, 60 bp, Artificial Sequence
  Synthetic
cctgcatttcaaccctcgcttcagcgaatccaccattgtctgcaactcattggacggcag

SEQ ID NO: 220 - DNA, 60 bp, Artificial Sequence
  Synthetic
gagagtgacaaattcaaggtgaagctgccagatgggcacgagctgacttttcccaacagg

SEQ ID NO: 221 - DNA, 60 bp, Artificial Sequence
  Synthetic
caggcagcatcgccgatggcactgatggctttgtaattaatctgggccaggggacagaca

SEQ ID NO: 222 - DNA, 60 bp, Artificial Sequence
  Synthetic
tgcaagagcaccccaacgcgcgcaaatacaaaggagccaacaagaagggcttgtccaagg

SEQ ID NO: 223 - DNA, 60 bp, Artificial Sequence
  Synthetic
ctcaagctggaccgaatcggctccatgagcggcctgggatgttagtgcggcgccccctgg

SEQ ID NO: 224 - DNA, 60 bp, Artificial Sequence
  Synthetic
atcgggaactggctccgttgtgctgaggtcatctttggtcatcagcctccagcatctgga

SEQ ID NO: 225 - DNA, 60 bp, Artificial Sequence
  Synthetic
cgggggcgccaatctcaagggcgaccggtcgcgactgctccgggacctgcgcgtggacac

SEQ ID NO: 226 - DNA, 60 bp, Artificial Sequence
  Synthetic
tgggagccagcctccctttgatgattattggagccccaggggacaagggatttgaggtga

SEQ ID NO: 227 - DNA, 60 bp, Artificial Sequence
  Synthetic
gtgtccttcttgctccccctcaatagatctccagcgtcagctgctccctggcattcaaca

SEQ ID NO: 228 - DNA, 60 bp, Artificial Sequence
  Synthetic
cctagggggagaagaagccgagagccttttgtgcaaagccaaaaccttcgtccttttaaa

SEQ ID NO: 229 - DNA, 60 bp, Artificial Sequence
  Synthetic
ggtccactgtgttaaggtcatttttaaccagcttgctttctacaccaagagtttatgttt

SEQ ID NO: 230 - DNA, 60 bp, Artificial Sequence
  Synthetic
gtatttagttgtgattactgattgcctgattttaaaatgttgccttctgggacatcttct

SEQ ID NO: 231 - DNA, 60 bp, Artificial Sequence
  Synthetic
cttctggagcatggtttacaaaagccagctgacttctggaattgtctatggaggacagtt

SEQ ID NO: 232 - DNA, 60 bp, Artificial Sequence
  Synthetic
actcggcagctccaagtggaatccacgtgcagcttctagtctgggaaagtcacccaacct

SEQ ID NO: 233 - DNA, 60 bp, Artificial Sequence
  Synthetic
cataatggtttctttctgaggttgcttcttggcctcagaggaccccaggggatgtttgga

SEQ ID NO: 234 - DNA, 60 bp, Artificial Sequence
  Synthetic
cagcttatttcctcatttttataatgtcccttcacaaacccagtgttttaggagcatgag

SEQ ID NO: 235 - DNA, 60 bp, Artificial Sequence
  Synthetic
aacctccacagaaccgccaagtccaaaattgaagacattagagcagaacaagagagagaa

SEQ ID NO: 236 - DNA, 60 bp, Artificial Sequence
  Synthetic
tagcgaggaggtgctgaagagcgcaggtttggagaatgatcacctggattggaaccatag

SEQ ID NO: 237 - DNA, 60 bp, Artificial Sequence
  Synthetic
agcctgctgacattgggtataagatcaagacactcatcaagaagtacatccagaggcagg

SEQ ID NO: 238 - DNA, 60 bp, Artificial Sequence
  Synthetic
agatgagctaggatggagagtccttgaacctgaacttacacaaatttgcctgtttctgct

SEQ ID NO: 239 - DNA, 60 bp, Artificial Sequence
  Synthetic
aggagcttactggcaaacatgaaaaatcggcttaccattaaagttctcaatgcaaccata

SEQ ID NO: 240 - DNA, 60 bp, Artificial Sequence
  Synthetic
tatcctaccccacccgctccttgaagggcccagattctaccacacagcagcagttacaaa

SEQ ID NO: 241 - DNA, 60 bp, Artificial Sequence
  Synthetic
ggtgacaaagtgagactccgtcacaacaacaacaacaaaaagcttccccaactaaagcct

SEQ ID NO: 242 - DNA, 60 bp, Artificial Sequence
  Synthetic
agctatgagttgaaatgttctgtcaaatgtgtctcacatctacacgtggcttggaggctt

SEQ ID NO: 243 - DNA, 60 bp, Artificial Sequence
  Synthetic
tcaagcaatccagcctcatgggaagtcctggcacagtttttgtaaagcccttgcacagct

SEQ ID NO: 244 - DNA, 60 bp, Artificial Sequence
  Synthetic
cactacaggaggaatgcaccacggcagctctccgccaatttctctcagatttccacagag

SEQ ID NO: 245 - DNA, 60 bp, Artificial Sequence
  Synthetic
aagaaatggtatgtagagcttagatttccctattgtgacagagccatggtgtgtttgtaa

SEQ ID NO: 246 - DNA, 60 bp, Artificial Sequence
  Synthetic
aaagcatttaggacttcataaagattttgatcagtgggactgcttgattgaaggagatga

SEQ ID NO: 247 - DNA, 60 bp, Artificial Sequence
  Synthetic
atgtaggctatactgtaatcgtgattgaagcttggactaagaattttttccctttagatg

SEQ ID NO: 248 - DNA, 60 bp, Artificial Sequence
  Synthetic
aatgctactcacctgataaagaaagaaagatgattacaatcaggggttcagtccaccaga

SEQ ID NO: 249 - DNA, 60 bp, Artificial Sequence
  Synthetic
aaatagcagtaattggacattcttttggtggagcaacggttattcagactcttagtgaag

SEQ ID NO: 250 - DNA, 60 bp, Artificial Sequence
  Synthetic
ataaagtctgtacatattggagctctgggagatgctggaataaaagacaagagttacatc

SEQ ID NO: 251 - DNA, 60 bp, Artificial Sequence
  Synthetic
catctggaggaaatggccttctttttaaaagcaaaaaacacaaaacctcacaactgcctg

SEQ ID NO: 252 - DNA, 60 bp, Artificial Sequence
  Synthetic
ggacttgtggatgggcctggactctccagaaactacttgggcagagcaaaggaaaacctc

SEQ ID NO: 253 - DNA, 60 bp, Artificial Sequence
  Synthetic
gatagccacgtgggccgagagctgacccgcaagaagggcatcctcttgcagtaccgcctg

SEQ ID NO: 254 - DNA, 60 bp, Artificial Sequence
  Synthetic
aaagctttgtattattcttccacatatgctggctgctgtttacacaccctgccaatgcct

SEQ ID NO: 255 - DNA, 60 bp, Artificial Sequence
  Synthetic
agtagagagaaacctacaaaatgtcaaaccagcttcccgactcccaggagctcaagccaa

SEQ ID NO: 256 - DNA, 60 bp, Artificial Sequence
  Synthetic
gtgaactgaggggagtagagggagagggcaggtggaactggggcagaatctagtcatgcc

SEQ ID NO: 257 - DNA, 60 bp, Artificial Sequence
  Synthetic
ggaaaacccacacacactccttggaatgggtcctgttatttatgcttgctgcacagacat

SEQ ID NO: 258 - DNA, 60 bp, Artificial Sequence
  Synthetic
ctccagaatggtctttctaatgtgtgaatcagagcacagcagcccctgcacaaagccctt

SEQ ID NO: 259 - DNA, 60 bp, Artificial Sequence
  Synthetic
tgcaaagttccctacttcctgtgacttcagctctgttttacaataaaatcttgaaaatgc

SEQ ID NO: 260 - DNA, 60 bp, Artificial Sequence
  Synthetic
cagcctcaccaatatgcctgacgaaggcgtcatggtcaccaaattctacttccaggagga

SEQ ID NO: 261 - DNA, 70 bp, Artificial Sequence
  Synthetic
ttgctccttgacattgtagagcttctggcacttggagacttgtatgaaagatggctgtgcctctgcctgt

SEQ ID NO: 262 - DNA, 60 bp, Artificial Sequence
  Synthetic
ttagaaacacttcaagaagaaaagcctgagctgaccgtcgtctttgagccttcttggtag

SEQ ID NO: 263 - DNA, 70 bp, Artificial Sequence
  Synthetic
agcatcgggtgttgttgtcatcacagcgcctcagttagaggatgttcctcttggtgacctcatgtaatta

SEQ ID NO: 264 - DNA, 60 bp, Artificial Sequence
  Synthetic
cgacaccttgatatggtgcagtgtgtcctcccaagctcctctcatgctgcctgttctcat

SEQ ID NO: 265 - DNA, 60 bp, Artificial Sequence
  Synthetic
ggagtccgacctcaggaatcatggactgcagaaggcggatgtgtctgctttcctgaggat

SEQ ID NO: 266 - DNA, 60 bp, Artificial Sequence
  Synthetic
tcagttcccataaaaccttcattacacataaagatacacgtgggggtcagtgaatctgct

SEQ ID NO: 267 - DNA, 60 bp, Artificial Sequence
  Synthetic
gtgcctggccaggcagtacaggctgatcgtccacaacggttactgcgatggcagatcaga

SEQ ID NO: 268 - DNA, 60 bp, Artificial Sequence
  Synthetic
tgcagtactcctgccgcctcctgaacctcgatggcacctgtgctgacagctactccttcg

SEQ ID NO: 269 - DNA, 60 bp, Artificial Sequence
  Synthetic
gtgcgcagacatggtgggcaccttcacagacaccgaggaccctgccaagttcaagatgaa

SEQ ID NO: 270 - DNA, 60 bp, Artificial Sequence
  Synthetic
taagtgggaagtcttgtttgttgttggtttttgtctgttgtttttcactgcacctctttg

SEQ ID NO: 271 - DNA, 60 bp, Artificial Sequence
  Synthetic
agccatgagaaggaaggagtggaatatcactttgtgtctaagcaagcatttgaggccgac

SEQ ID NO: 272 - DNA, 60 bp, Artificial Sequence
  Synthetic
gactgtgagggctacctcaaagggcactatgtggctggtcttcggaggagcttccggctg

SEQ ID NO: 273 - DNA, 60 bp, Artificial Sequence
  Synthetic
gcctgacaatatcgatgaggattttgatgaggaatcggtgaagatcgtccgcttggtgaa

SEQ ID NO: 274 - DNA, 56 bp, Artificial Sequence
  Synthetic
agaggaaggagctcttagttacccttttgtgttgcccttctttccatcaaggggaa

SEQ ID NO: 275 - DNA, 60 bp, Artificial Sequence
  Synthetic
ctggagacctttgtgaacaaagcggaatctgctctggcccagcaagccaagcatttctca

SEQ ID NO: 276 - DNA, 60 bp, Artificial Sequence
  Synthetic
ggagagcagttgattcaaatggaaacagaagagatggaagcctgctctaacggggcgctg

SEQ ID NO: 277 - DNA, 60 bp, Artificial Sequence
  Synthetic
gtatctggagaaccaagcattctgctttgactttgcatttgatgaaacagcttcgaatga

SEQ ID NO: 278 - DNA, 60 bp, Artificial Sequence
  Synthetic
aatgccagaattcttccaaactccctgactctttgaagtttttactcaccccatttcaat

SEQ ID NO: 279 - DNA, 60 bp, Artificial Sequence
  Synthetic
gctttataccattcacatcccagggctgtgtccagacagcacaaaacggcaaggagagcc

SEQ ID NO: 280 - DNA, 60 bp, Artificial Sequence
  Synthetic
cacgcttccctgctatagttcccagctgctgtaacggagccacctccaactctaacaata

SEQ ID NO: 281 - DNA, 60 bp, Artificial Sequence
  Synthetic
cccctgcttggcttctctgcatgtggtcatctgctgtggcttggtgtttaatgggttaaa

SEQ ID NO: 282 - DNA, 60 bp, Artificial Sequence
  Synthetic
ataggtctgataatgggtgggacgctcctggctttgctaagaaagcgtttcagacaaaaa

SEQ ID NO: 283 - DNA, 60 bp, Artificial Sequence
  Synthetic
aaatacctctttattttttgattgaaggaaggttttctccactttgttggaaagcaggtg

SEQ ID NO: 284 - DNA, 60 bp, Artificial Sequence
  Synthetic
gtcagctactccaccaacctgcaaaggcatagcatcacttcctactccaggggtgcaatg

SEQ ID NO: 285 - DNA, 60 bp, Artificial Sequence
  Synthetic
aattctcaacctaccaccccttcctgtcccacctcttctcttcctgtaacacaagccaca

SEQ ID NO: 286 - DNA, 53 bp, Artificial Sequence
  Synthetic
tgagcaggatatgttagaaaagaggaaaatacagcgtgaattagcaaaaaggg

SEQ ID NO: 287 - DNA, 55 bp, Artificial Sequence
  Synthetic
gcggggccaaaaaaggaaaccaggagtgccactatgctgacttcttattcctttt

SEQ ID NO: 288 - DNA, 60 bp, Artificial Sequence
  Synthetic
agagaagatgaaaaaaggaaagcagaagaagaagccagaaggagaatagaggaagaaaag

SEQ ID NO: 289 - DNA, 60 bp, Artificial Sequence
  Synthetic
gaaggtagcatcatgaatggctccactgctgaagatgaagagcaaaccagatcaggagct

SEQ ID NO: 290 - DNA, 60 bp, Artificial Sequence
  Synthetic
acaacaatggccctgggactgtgaatggtcttcacctcagcatccaccttccgggacagt

SEQ ID NO: 291 - DNA, 60 bp, Artificial Sequence
  Synthetic
tcgtaagctgcgactcggcgccctgtactgtggtgcagtgtgacctgcaggagatggcgc

SEQ ID NO: 292 - DNA, 60 bp, Artificial Sequence
  Synthetic
gtgagctgggcaaccccatgaagaagaacgcccagataggaatcgcgatgttggtgagcg

SEQ ID NO: 293 - DNA, 60 bp, Artificial Sequence
  Synthetic
gggccttggaggagagggccattccaatctggtgggtgctggtgggtgtgctgggtggcc

SEQ ID NO: 294 - DNA, 60 bp, Artificial Sequence
  Synthetic
tgaggaagtggacgagatgtaccgggaggcacccattgataagaaaggcaacttcaacta

SEQ ID NO: 295 - DNA, 60 bp, Artificial Sequence
  Synthetic
cctccccgcacacacccgtccataccagctccctgcccatgaccctcgctcagggatccc

SEQ ID NO: 296 - DNA, 60 bp, Artificial Sequence
  Synthetic
cttcacgtgtatccccacacaaatgcaagctcaccaaggtcccctctcagtccccttccc

SEQ ID NO: 297 - DNA, 60 bp, Artificial Sequence
  Synthetic
tcaaacatggcgccaaggataaagacgactaggccaccccagccccctgacaccccagcc

SEQ ID NO: 298 - DNA, 60 bp, Artificial Sequence
  Synthetic
atgtgtggacacattggacctttcctgaggaagagggactgttcttttgtcccagaaaag

SEQ ID NO: 299 - DNA, 60 bp, Artificial Sequence
  Synthetic
ataagtagctgaaatatctattctgtattattgtgttaacattgagaataagccttggaa

SEQ ID NO: 300 - DNA, 60 bp, Artificial Sequence
  Synthetic
tcccatgagttggctgggaataagtgccaggatggaatgatgggtcagttgtatcagcac

SEQ ID NO: 301 - DNA, 60 bp, Artificial Sequence
  Synthetic
ccctcaacccagctatggttctctcgcaagggaagtccttgcaagctaattctttgacct

SEQ ID NO: 302 - DNA, 60 bp, Artificial Sequence
  Synthetic
tgagacgtcactggggacttatctgtggagcctggtgctccaggatgtggcttctcatga

SEQ ID NO: 303 - DNA, 60 bp, Artificial Sequence
  Synthetic
ggcatcctgctggaaaccgagctggccctgaccctcatcatcttcatctgcttcacggcc

SEQ ID NO: 304 - DNA, 60 bp, Artificial Sequence
  Synthetic
gcagtagggggctgtgttggtgggccctacgaagatgctcagtgctcgagatcgccggga

SEQ ID NO: 305 - DNA, 60 bp, Artificial Sequence
  Synthetic
cctaggcccagccagccagagaggacagtggagcccagacacgtctccttgggattcact

SEQ ID NO: 306 - DNA, 60 bp, Artificial Sequence
  Synthetic
gctatggtgttcttcaagaaagtttctcaaaacagggagtacttcaagatcaccctctac

SEQ ID NO: 307 - DNA, 60 bp, Artificial Sequence
  Synthetic
ctgaaaaccacatcgtcttccctgtcccaatcgaccagtgtatcgacggctgagtgcaca

SEQ ID NO: 308 - DNA, 60 bp, Artificial Sequence
  Synthetic
caaaagatgtatgccaccatctatgagctgaaagaagacaagagctacaatgtcacctcc

SEQ ID NO: 309 - DNA, 60 bp, Artificial Sequence
  Synthetic
tctgcatgcccaggcccaggactccacctcagacctgatcccagccccacctctgagcaa

SEQ ID NO: 310 - DNA, 60 bp, Artificial Sequence
  Synthetic
ttcttaaatcccttaaggagtggaactatcctctatttcaggacttgaatggacaaagtc

SEQ ID NO: 311 - DNA, 60 bp, Artificial Sequence
  Synthetic
aacttgaaaagcaccttcacagaacctgtcctgtggaggaaggaccaacaccatcaccgc

SEQ ID NO: 312 - DNA, 60 bp, Artificial Sequence
  Synthetic
cctctgtgatcaactcctggatatacctggcacaatcaggaagcagacattcatggccat

SEQ ID NO: 313 - DNA, 60 bp, Artificial Sequence
  Synthetic
ggaatctcatgaagacccctctctttgtggtcatcacttgtgcaatccagatgggtgaaa

SEQ ID NO: 314 - DNA, 60 bp, Artificial Sequence
  Synthetic
gacagtgacttgtatgctgaactccgctgcatgtgtataaagacaacctctggaattcat

SEQ ID NO: 315 - DNA, 60 bp, Artificial Sequence
  Synthetic
agatgaaaataaataagcctggtttcaaccctctaattcttgcctaaacattggactgta

SEQ ID NO: 316 - DNA, 60 bp, Artificial Sequence
  Synthetic
gtcgaagtgatagccacactgaaggatgggaggaaaatctgcctggacccagatgctccc

SEQ ID NO: 317 - DNA, 60 bp, Artificial Sequence
  Synthetic
ctcagtgtcttagtcctaggatgtcttatttaaaatactccctgaaagtttattctgatg

SEQ ID NO: 318 - DNA, 60 bp, Artificial Sequence
  Synthetic
tctgctccaagcctgtgacatatgccacagtaatcttcccgggagggaacaagggtggag

SEQ ID NO: 319 - DNA, 60 bp, Artificial Sequence
  Synthetic
tcttaatggctgaatgggaaaggaaactgcccaagtttgactaattgcttggcctgtgaa

SEQ ID NO: 320 - DNA, 60 bp, Artificial Sequence
  Synthetic
tcagactccatccagctaagctgctcatcacactttaaactcatgaggaccatccctagg

SEQ ID NO: 321 - DNA, 60 bp, Artificial Sequence
  Synthetic
gaattgcctttggatgtaccacacattaggcttgactcaccaccttcatttgacaatacc

SEQ ID NO: 322 - DNA, 60 bp, Artificial Sequence
  Synthetic
gacctgcaagccccgctgtacaagaaaataattaagaaacttttggagagttagctacta

SEQ ID NO: 323 - DNA, 60 bp, Artificial Sequence
  Synthetic
tttgaaggaaggtgtgaatactggttatgcttggtgttacatgttggctgatacatattc

SEQ ID NO: 324 - DNA, 60 bp, Artificial Sequence
  Synthetic
gattgcagtactttatagctacatatttaccttgaccattattattacctttgccaataa

SEQ ID NO: 325 - DNA, 60 bp, Artificial Sequence
  Synthetic
caatctaactgtgaaagaacttctgatatttgtgttatccttatgattttaaataaacaa

SEQ ID NO: 326 - DNA, 60 bp, Artificial Sequence
  Synthetic
aaacattatttaatgtgtgagaggaaggctggcatgaccaaggtggaccaactaccttaa

SEQ ID NO: 327 - DNA, 60 bp, Artificial Sequence
  Synthetic
ggtggacaggataacacagataagggctttattgtacaataaaagatatgtatgaatgca

SEQ ID NO: 328 - DNA, 60 bp, Artificial Sequence
  Synthetic
ggtctgtcatgcagcgcaattacctacaaggtgagaatgaaaatcgcacaggaactctgc

SEQ ID NO: 329 - DNA, 60 bp, Artificial Sequence
  Synthetic
tgtgacacaaactggagatattatggagatagctgctatgggttcttcaggcacaactta

SEQ ID NO: 330 - DNA, 60 bp, Artificial Sequence
  Synthetic
gcctgtgggatgccttgtgggacgtctctttctattcaataaacagatgctgcagcctca

SEQ ID NO: 331 - DNA, 60 bp, Artificial Sequence
  Synthetic
tgcgcatcgtggacacagactacagctccttcgccgtcctttacatctacaaggagctgg

SEQ ID NO: 332 - DNA, 60 bp, Artificial Sequence
  Synthetic
cagcaccatggtgcagctctacagccggacccaggatgtgagtccccaggctctgaagtc

SEQ ID NO: 333 - DNA, 60 bp, Artificial Sequence
  Synthetic
cggctgtaaccaggtggatgccgagtacctgaaggtgggctccgagggacacttcagagt

SEQ ID NO: 334 - DNA, 60 bp, Artificial Sequence
  Synthetic
gcagcggcgtcaaagtggtcaccttctgtggccacacgtccaaaaccaatcaggtcaact

SEQ ID NO: 335 - DNA, 60 bp, Artificial Sequence
  Synthetic
catggtgggcatccagggctctgacagcgtcttctccggcttcctgctcttccccgacta

SEQ ID NO: 336 - DNA, 60 bp, Artificial Sequence
  Synthetic
agtcactgcttgtgtggttcctgggacacttaaccaatgccttctggtactgccattctt

SEQ ID NO: 337 - DNA, 60 bp, Artificial Sequence
  Synthetic
ccttccacctccctcagcttcctgcatggacccaccttactggccagtctgcatccttgc

SEQ ID NO: 338 - DNA, 60 bp, Artificial Sequence
  Synthetic
caccgacaagaactcactactgggcatggagggtgccaacagcatcttttccgggttcct

SEQ ID NO: 339 - DNA, 60 bp, Artificial Sequence
  Synthetic
gccagcaacgctcactctacccccaacaccaccccttgcccaaccaatgcacacagtagg

SEQ ID NO: 340 - DNA, 60 bp, Artificial Sequence
  Synthetic
gaccagaccatccgcttcgaccacgtgatcaccaacatgaacaacaattatgagccccgc

SEQ ID NO: 341 - DNA, 60 bp, Artificial Sequence
  Synthetic
cctcatgcgtggccgggagcgtgcacagaaggtggtcaccttctgtgactatgcctacaa

SEQ ID NO: 342 - DNA, 60 bp, Artificial Sequence
  Synthetic
tatctgattggaaacctgccgacttagtgcggtgataggaagctaaaagtgtcaagcgtt

SEQ ID NO: 343 - DNA, 60 bp, Artificial Sequence
  Synthetic
aactgtgtccatttaagctgtattctgccattgcctttgaaagatctatgttctctcagt

SEQ ID NO: 344 - DNA, 60 bp, Artificial Sequence
  Synthetic
catcaacatctacaaagagggaaatttggcgattcagcaggcgggcaagaacatgagtgc

SEQ ID NO: 345 - DNA, 60 bp, Artificial Sequence
  Synthetic
agtgtagacggacggggactctggagggcaattattgttcaagtgactttgtattagccg

SEQ ID NO: 346 - DNA, 60 bp, Artificial Sequence
  Synthetic
gggtgaccaggtctgggttgaaaaagaccccaaaaagggtcacatttaccagggctctga

SEQ ID NO: 347 - DNA, 60 bp, Artificial Sequence
  Synthetic
ctgcactgtacccggctactactacttcaccttccaggtgctgtcccagtgggaaatctg

SEQ ID NO: 348 - DNA, 60 bp, Artificial Sequence
  Synthetic
tcccccacccacctctctggcttccatgctccgcctgtaaaatgggggcgctattgcttc

SEQ ID NO: 349 - DNA, 60 bp, Artificial Sequence
  Synthetic
gcagcccaggaaacatcaaggaccagccgaggccagccttctccgccattcggcggaacc

SEQ ID NO: 350 - DNA, 60 bp, Artificial Sequence
  Synthetic
agcacgtctgtacttctgttcattaaagtgctccctttctagtcctttttctgcccagaa

SEQ ID NO: 351 - DNA, 60 bp, Artificial Sequence
  Synthetic
tctagacctgctggactctgcagggggtgagggggaacagcgagagcttgggtaatgatt

SEQ ID NO: 352 - DNA, 60 bp, Artificial Sequence
  Synthetic
atccctggggctcccagggttgttaagaatggatcattcttccagctaagggtccaatca

SEQ ID NO: 353 - DNA, 60 bp, Artificial Sequence
  Synthetic
ttcctgaacgccatcagcggccttcacatcaacagcatcacccatccctgggaatgaccg

SEQ ID NO: 354 - DNA, 60 bp, Artificial Sequence
  Synthetic
ggggtatcctgggggacccaatgtaggagctgccttggctcagacatgttttccgtgaaa

SEQ ID NO: 355 - DNA, 60 bp, Artificial Sequence
  Synthetic
ccagaactcactggggcctacagctttgatccctgacatctggaatctggagaccaggga

SEQ ID NO: 356 - DNA, 60 bp, Artificial Sequence
  Synthetic
cagccctccccatggagccagctccctctatttatgtttgcacttgtgattatttattat

SEQ ID NO: 357 - DNA, 60 bp, Artificial Sequence
  Synthetic
aggggagttgtgtctgtaatcgccctactattcagtggcgagaaataaagtttgcttaga

SEQ ID NO: 358 - DNA, 60 bp, Artificial Sequence
  Synthetic
ggaaaatgattgacaaagcccaacaatgatctcaggaattacattttccaacagaccaaa

SEQ ID NO: 359 - DNA, 60 bp, Artificial Sequence
  Synthetic
accatgtgtttgctttgtgaaggtgaagaatatgttggtttagagaaagaaattggatgt

SEQ ID NO: 360 - DNA, 60 bp, Artificial Sequence
  Synthetic
tcaagagcaagtacatcaaaatgtagaaggtaaaatgtatgcaacactaatataaattat

SEQ ID NO: 361 - DNA, 60 bp, Artificial Sequence
  Synthetic
aattagcacaccatggttatttttctaccttttataaaagacagagcctgtttactcatt

SEQ ID NO: 362 - DNA, 60 bp, Artificial Sequence
  Synthetic
ggacattaaaggaatttcccttttgttgacataacagtaggatggctatagctaacaata

SEQ ID NO: 363 - DNA, 60 bp, Artificial Sequence
  Synthetic
ctactgtcctttgtgcaaccagtatagaagcgattgactctcactgcatccttgggtctt

SEQ ID NO: 364 - DNA, 60 bp, Artificial Sequence
  Synthetic
tctgaacctggccctctgaaacttcctgttcctggacatcccagctgctaatgtgagttg

SEQ ID NO: 365 - DNA, 60 bp, Artificial Sequence
  Synthetic
gccatttttatactttacttatggtagagttaaacttcgtactcttaggaggctacatgc

SEQ ID NO: 366 - DNA, 60 bp, Artificial Sequence
  Synthetic
ttgtgtttgaaattccatgtcgggtttacttggaatgaaagatacttgaattattgtgcg

SEQ ID NO: 367 - DNA, 60 bp, Artificial Sequence
  Synthetic
tagtgctaccctcacatcccctggagcacagccttcctgaaatgccctcaccccatgcct

SEQ ID NO: 368 - DNA, 60 bp, Artificial Sequence
  Synthetic
agtgggccctcatgactgaggtagcttccagataggccagagtagagtgtagagtgtgcc

SEQ ID NO: 369 - DNA, 60 bp, Artificial Sequence
  Synthetic
gccactcgggacacctggatggtttctcttaggactttgcccacctccttctcatggcac

SEQ ID NO: 370 - DNA, 60 bp, Artificial Sequence
  Synthetic
actttgagaactctgtgagacaaggtccttaggcacccagatatcagccactttcacatt

SEQ ID NO: 371 - DNA, 60 bp, Artificial Sequence
  Synthetic
gagccaaagtcaaggtattgatgacgctacactcctccggaggctctaggcagatagcct

SEQ ID NO: 372 - DNA, 60 bp, Artificial Sequence
  Synthetic
cattattcttaccacagagcaccccaagaaaatctccaaattttgggcttccaatccatt

SEQ ID NO: 373 - DNA, 60 bp, Artificial Sequence
  Synthetic
catattcttcccaaacctcatgcagtttacaatctagtgagagacacagatagcagtaca

SEQ ID NO: 374 - DNA, 60 bp, Artificial Sequence
  Synthetic
aaggatgttctaattctttctgctctgagacgaatgctatgggctgcagatgacttctta

SEQ ID NO: 375 - DNA, 60 bp, Artificial Sequence
  Synthetic
aaggtgaccttatagaaatagagagatgtgagcctgtgaggtccaagctagaggaagtcc

SEQ ID NO: 376 - DNA, 60 bp, Artificial Sequence
  Synthetic
attaaaattcagaaaggagaaaacacagaccaaagagaagtatctaagaccaaagggatg

SEQ ID NO: 377 - DNA, 60 bp, Artificial Sequence
  Synthetic
aattcgaagggagttggtaaacgctggtgtggtacatgtggctttgctcactcatgtgga

SEQ ID NO: 378 - DNA, 60 bp, Artificial Sequence
  Synthetic
ccgaaggcggtttctgttcgacatgctgaatggacaagatcctgatcaaggacatttaaa

SEQ ID NO: 379 - DNA, 60 bp, Artificial Sequence
  Synthetic
ggcagatcctggagtcaggaagagatattctagagcaagagctagacattgttagccctg

SEQ ID NO: 380 - DNA, 60 bp, Artificial Sequence
  Synthetic
gctcgattcccagtgcttgtgacctctgacctggaccattatctgagacgggaggccttg

SEQ ID NO: 381 - DNA, 60 bp, Artificial Sequence
  Synthetic
tccctacataccccctgagagcttcctgaaaccagaagctcagactaaggggaaggatac

SEQ ID NO: 382 - DNA, 60 bp, Artificial Sequence
  Synthetic
gtgaatttgagaaatgtcagactgtgccaaagggggtcacatgcatcaaatttccacata

SEQ ID NO: 383 - DNA, 60 bp, Artificial Sequence
  Synthetic
tgatatgctagggaatatgggagacaataggaaagtaagacagacatggtatctgccctt

SEQ ID NO: 384 - DNA, 60 bp, Artificial Sequence
  Synthetic
ccacctgtctgcccatggtgtgatgtatctataatccacttgttcataaaaaatattcgt

SEQ ID NO: 385 - DNA, 60 bp, Artificial Sequence
  Synthetic
gtcatcatgtcccaattttctttccatctttactcatatctaccttttgaatcccaaaag

SEQ ID NO: 386 - DNA, 55 bp, Artificial Sequence
  Synthetic
ccgaaggcggtttctgttcgacatgctgaatggacaagatcctgatcaaggacat

SEQ ID NO: 387 - DNA, 60 bp, Artificial Sequence
  Synthetic
ggggactgtctatgaatctgtccctgcccttcttccttttccgccaggcttaccatccaa

SEQ ID NO: 388 - DNA, 60 bp, Artificial Sequence
  Synthetic
cctgcctcacacctttggcttcatcgtgccgctgtttgtcatgctgttctgctatggatt

SEQ ID NO: 389 - DNA, 60 bp, Artificial Sequence
  Synthetic
gtccattgggcaggcagatgttcctaataaagcttctgttccgtgcttgtccctgtggaa

SEQ ID NO: 390 - DNA, 60 bp, Artificial Sequence
  Synthetic
attgtcaaggtgttgccgccggtgttggggctggagtttatcttcgggcttctgggcaat

SEQ ID NO: 391 - DNA, 60 bp, Artificial Sequence
  Synthetic
gtgggagactggttgcaaggtgtgaccgcaggaatcctggaggaatagagagtaaagctt

SEQ ID NO: 392 - DNA, 60 bp, Artificial Sequence
  Synthetic
agatgacaggtgagccagataataaccgcagcacgagcgtcgagctcacaggggacccca

SEQ ID NO: 393 - DNA, 60 bp, Artificial Sequence
  Synthetic
agcagaaatggactcagggaagagactcacacgctttggttaatatctgtgtttccggtg

SEQ ID NO: 394 - DNA, 60 bp, Artificial Sequence
  Synthetic
agaagagaagcatttagagagcatcagcaatacaaaaccgctgagttcttgagcaaactg

SEQ ID NO: 395 - DNA, 60 bp, Artificial Sequence
  Synthetic
ctaccttacagttgtgttccgatttttgcgtctatgcttggtgtgcctcacttgctgcat

SEQ ID NO: 396 - DNA, 60 bp, Artificial Sequence
  Synthetic
ggccacattggtgggtggggggtaacagagaacagatggtgtcaggaagtttctctggag

SEQ ID NO: 397 - DNA, 60 bp, Artificial Sequence
  Synthetic
taaccaggagagaagcaagacttgctgcctaaaggagcccaccattttacttttcacatt

SEQ ID NO: 398 - DNA, 60 bp, Artificial Sequence
  Synthetic
tgaagccttcttcctcccaggaaaggggacgttcagctgagccgagtgtgtatactgctc

SEQ ID NO: 399 - DNA, 60 bp, Artificial Sequence
  Synthetic
aaatattacacatcaaaccaatgacatgggaaaatgggagcttctaatgaggacaaacaa

SEQ ID NO: 400 - DNA, 60 bp, Artificial Sequence
  Synthetic
atggagactcaggaccccagaaggcatggaagctgcctccagtagacatcactgaacccc

SEQ ID NO: 401 - DNA, 60 bp, Artificial Sequence
  Synthetic
aagaacacacagcctgaggacggggtggaaatggacactcggcagagcccacacgatgaa

SEQ ID NO: 402 - DNA, 60 bp, Artificial Sequence
  Synthetic
ctagattctgcagtcaaagatgactaatatccttgcatttttgaaatgaagccacagact

SEQ ID NO: 403 - DNA, 60 bp, Artificial Sequence
  Synthetic
ccaaggccaagttccacattccatccacggtgtatgacagtgcagggcgataccgctgct

SEQ ID NO: 404 - DNA, 60 bp, Artificial Sequence
  Synthetic
tcaggaggaaatgtgaccctccagtgtgatacactggacggacttctcacgtttgttctt

SEQ ID NO: 405 - DNA, 60 bp, Artificial Sequence
  Synthetic
tgtctaaagtcaaagtaccagtcttatagacaccaggctgaattctccatgagtcctgtg

SEQ ID NO: 406 - DNA, 60 bp, Artificial Sequence
  Synthetic
acggctcatgtttttaaaatatatgtaactcattttaaaatatattaaattgtattccaa

SEQ ID NO: 407 - DNA, 60 bp, Artificial Sequence
  Synthetic
tccattacagttattgttgctagatccacctcatttgcagatgtccaaacttaaattcat

SEQ ID NO: 408 - DNA, 60 bp, Artificial Sequence
  Synthetic
acttcccaatttatgcaggaaacctcctgcaaagctgaaactgattagaaaattctttat

SEQ ID NO: 409 - DNA, 60 bp, Artificial Sequence
  Synthetic
gtagcaaatattttatatttgagagtctttacagcttacatttccattccattattacaa

SEQ ID NO: 410 - DNA, 60 bp, Artificial Sequence
  Synthetic
ttttttctagacagacactttggtgcagaagcttcttttcaatccatcctccacaagaag

SEQ ID NO: 411 - DNA, 60 bp, Artificial Sequence
  Synthetic
agcacccctgcctcctggtcctggcctctcccctacccctcacatggccacgcatgaccc

SEQ ID NO: 412 - DNA, 60 bp, Artificial Sequence
  Synthetic
tgcagctggtccggcaggagatggccgtgtgccccgagcaactgagcgagtttctggact

SEQ ID NO: 413 - DNA, 60 bp, Artificial Sequence
  Synthetic
ctgggagacagaggaggcgtggaagaggcacctgcagagccccctgtgtaaggcgttccg

SEQ ID NO: 414 - DNA, 60 bp, Artificial Sequence
  Synthetic
gtacgtgtcaataacagtaaagaattttaattaattctgtcatttaaaattcatgcattc

SEQ ID NO: 415 - DNA, 60 bp, Artificial Sequence
  Synthetic
accagtttctgtatttcctaaaacagaggcagaatggactgcatccttcttcaacgcagg

SEQ ID NO: 416 - DNA, 60 bp, Artificial Sequence
  Synthetic
ttccatctatcagtcttactccaaatatatgaaattactatacattgaaatgcctgcaaa

SEQ ID NO: 417 - DNA, 60 bp, Artificial Sequence
  Synthetic
aattagggaaacaccaaaagcaaagagagcagctctatgttcattcatgtctggagatga

SEQ ID NO: 418 - DNA, 60 bp, Artificial Sequence
  Synthetic
ctatgcgctatggagagagttcctcttctccgttcccgcggggccccccgcgcagtactc

SEQ ID NO: 419 - DNA, 60 bp, Artificial Sequence
  Synthetic
acgtgagcaacgtcaccgtgaactacaacatcaccgtggagcggatgaacaggatgcagg

SEQ ID NO: 420 - DNA, 60 bp, Artificial Sequence
  Synthetic
aagacactggtgctggatgagaccaccacatccacgggcagcgcaggcatgtgactggtg

SEQ ID NO: 421 - DNA, 60 bp, Artificial Sequence
  Synthetic
tggctgtgcctgcttctgtgtaccacttctgtgggcatggccgcttctagagcctcgaca

SEQ ID NO: 422 - DNA, 23 bp, Artificial Sequence
  Forward primer 1 FAM20A
gccatcttcgacttcttgatagg

SEQ ID NO: 423 - DNA, 21 bp, Artificial Sequence
  Reverse primer 1 FAM20A
cccgaacttggtgaacatctc

SEQ ID NO: 424 - DNA, 18 bp, Artificial Sequence
  Synthetic 1 FAM20A
aatatggaccggcaccat

SEQ ID NO: 425 - DNA, 20 bp, Artificial Sequence
  Forward primer 2 FAM20A
agcggctcctcaatgtcatc

SEQ ID NO: 426 - DNA, 22 bp, Artificial Sequence
  Reverse primer 2 FAM20A
ccggtccatattccctatcaag

SEQ ID NO: 427 - DNA, 17 bp, Artificial Sequence
  Synthetic 2 FAM20A
catggccatcttcgact

SEQ ID NO: 428 - DNA, 22 bp, Artificial Sequence
  Forward primer 1 OLAH
acatggaagcctggaaagatgt

SEQ ID NO: 429 - DNA, 21 bp, Artificial Sequence
  Reverse primer 1 OLAH
cccctggaagctggtaaattt

SEQ ID NO: 430 - DNA, 14 bp, Artificial Sequence
  Synthetic 1 OLAH
accagtggaaatgc

SEQ ID NO: 431 - DNA, 24 bp, Artificial Sequence
  Forward primer 2 OLAH
tttgtcaagtgcaactcctgtaca

SEQ ID NO: 432 - DNA, 24 bp, Artificial Sequence
  Reverse primer 2 OLAH
gacaattcatcatctttgggaatg

SEQ ID NO: 433 - DNA, 16 bp, Artificial Sequence
  Synthetic 2 OLAH
tcaaaggcctggcatc

SEQ ID NO: 434 - DNA, 18 bp, Artificial Sequence
  Forward primer 1 LCN2
tgggcctccctgaaaacc

SEQ ID NO: 435 - DNA, 21 bp, Artificial Sequence
  Reverse primer 1 LCN2
ccgtcgatacactggtcgatt

SEQ ID NO: 436 - DNA, 17 bp, Artificial Sequence
  Synthetic 1 LCN2
catcgtcttccctgtcc

SEQ ID NO: 437 - DNA, 26 bp, Artificial Sequence
  Forward primer 2 LCN2
gtttctcaaaacagggagtacttcaa

SEQ ID NO: 438 - DNA, 24 bp, Artificial Sequence
  Reverse primer 2 LCN2
ctttagttccgaagtcagctcctt

SEQ ID NO: 439 - DNA, 18 bp, Artificial Sequence
  Synthetic 2 LCN2
atcaccctctacgggaga

SEQ ID NO: 440 - DNA, 22 bp, Artificial Sequence
  Forward primer 1 ITGB3
gtcctccagctcattgttgatg

SEQ ID NO: 441 - DNA, 21 bp, Artificial Sequence
  Reverse primer 1 ITGB3
ggtcacgcacttccagctcta

SEQ ID NO: 442 - DNA, 21 bp, Artificial Sequence
  Synthetic 1 ITGB3
ttatgggaaaatccgttctaa

SEQ ID NO: 443 - DNA, 23 bp, Artificial Sequence
  Forward primer 2 ITGB3
acgaaaatacctgcaaccgttac

SEQ ID NO: 444 - DNA, 23 bp, Artificial Sequence
  Reverse primer 2 ITGB3
ttgccagtgtccttaagctcttt

SEQ ID NO: 445 - DNA, 17 bp, Artificial Sequence
  Synthetic 2 ITGB3
cgtgacgagattgagtc

SEQ ID NO: 446 - DNA, 18 bp, Artificial Sequence
  Forward primer 1 MYL9
gagcccaagcgccttctc

SEQ ID NO: 447 - DNA, 19 bp, Artificial Sequence
  Reverse primer 1 MYL9
cccgcttgctggacatctt

SEQ ID NO: 448 - DNA, 15 bp, Artificial Sequence
  Synthetic 1 MYL9
accagggaagcccca

SEQ ID NO: 449 - DNA, 15 bp, Artificial Sequence
  Forward primer 2 MYL9
gccggcccagttcca

SEQ ID NO: 450 - DNA, 18 bp, Artificial Sequence
  Reverse primer 2 MYL9
cttccctggtgcggagaa

SEQ ID NO: 451 - DNA, 14 bp, Artificial Sequence
  Synthetic 2 MYL9
acccagcgagccca

SEQ ID NO: 452 - DNA, 20 bp, Artificial Sequence
  Forward primer 1 TREML1
acccagccaggatgagaaga

SEQ ID NO: 453 - DNA, 19 bp, Artificial Sequence
  Reverse primer 1 TREML1
gccaccagcagacctacca

SEQ ID NO: 454 - DNA, 16 bp, Artificial Sequence
  Synthetic 1 TREML1
atccccttgatctggg

SEQ ID NO: 455 - DNA, 19 bp, Artificial Sequence
  Forward primer 2 TREML1
cagtgccaaccctttggaa

SEQ ID NO: 456 - DNA, 19 bp, Artificial Sequence
  Reverse primer 2 TREML1
ggagcacagcaccccagat

SEQ ID NO: 457 - DNA, 14 bp, Artificial Sequence
  Synthetic 2 TREML1
ccagccaggatgag

SEQ ID NO: 458 - DNA, 18 bp, Artificial Sequence
  Forward primer 1 ITGA2B
ctgggcaaccccatgaag

SEQ ID NO: 459 - DNA, 18 bp, Artificial Sequence
  Reverse primer 1 ITGA2B
ccccacgctcaccaacat

SEQ ID NO: 460 - DNA, 15 bp, Artificial Sequence
  Synthetic 1 ITGA2B
aacgcccagatagga

SEQ ID NO: 461 - DNA, 19 bp, Artificial Sequence
  Forward primer 2 ITGA2B
cccttcgaggtgccgtaga

SEQ ID NO: 462 - DNA, 20 bp, Artificial Sequence
  Reverse primer 2 ITGA2B
ccgtaagctcccacgatcag

SEQ ID NO: 463 - DNA, 18 bp, Artificial Sequence
  Synthetic 2 ITGA2B
tcgatgacaacggatacc

SEQ ID NO: 464 - DNA, 23 bp, Artificial Sequence
  Forward primer 1 PLA2G7
tgttgcccatatgaaatcatcag

SEQ ID NO: 465 - DNA, 19 bp, Artificial Sequence
  Reverse primer 1 PLA2G7
aagcttgcagcagccatca

SEQ ID NO: 466 - DNA, 21 bp, Artificial Sequence
  Synthetic 1 PLA2G7
atgggtcaacaaaatacaagt

SEQ ID NO: 467 - DNA, 21 bp, Artificial Sequence
  Forward primer 2 PLA2G7
acactggcttatgggcaacat

SEQ ID NO: 468 - DNA, 23 bp, Artificial Sequence
  Reverse primer 2 PLA2G7
attccagtttgcaggagttgtca

SEQ ID NO: 469 - DNA, 21 bp, Artificial Sequence
  Synthetic 2 PLA2G7
ttgaggttactctttggttca

SEQ ID NO: 470 - DNA, 20 bp, Artificial Sequence
  Forward primer 1 ARHGEF10L
atccactcggccaacaagtg

SEQ ID NO: 471 - DNA, 18 bp, Artificial Sequence
  Reverse primer 1 ARHGEF10L
gccggacttgtcgggttt

SEQ ID NO: 472 - DNA, 15 bp, Artificial Sequence
  Synthetic 1 ARHGEF10L
cgtctcaggctcctg

SEQ ID NO: 473 - DNA, 22 bp, Artificial Sequence
  Forward primer 1 TGFBI
ggtccatgtcatcaccaatgtt

SEQ ID NO: 474 - DNA, 21 bp, Artificial Sequence
  Reverse primer 1 TGFBI
cccctctttcctgaggtctgt

SEQ ID NO: 475 - DNA, 14 bp, Artificial Sequence
  Synthetic 1 TGFBI
tgcagcctccagcc

SEQ ID NO: 476 - DNA, 19 bp, Artificial Sequence
  Forward primer 2 TGFBI
ggcgtggtccatgtcatca

SEQ ID NO: 477 - DNA, 22 bp, Artificial Sequence
  Reverse primer 2 TGFBI
ccctctttcctgaggtctgttg

SEQ ID NO: 478 - DNA, 15 bp, Artificial Sequence
  Synthetic 2 TGFBI
tgttctgcagcctcc

SEQ ID NO: 479 - DNA, 17 bp, Artificial Sequence
  Forward primer 1 MYCL
gctgggcgaacccaaga

SEQ ID NO: 480 - DNA, 28 bp, Artificial Sequence
  Reverse primer 1 MYCL
tcttctctactgtcacaacatcaatttc

SEQ ID NO: 481 - DNA, 13 bp, Artificial Sequence
  Synthetic 1 MYCL
ccaggcctgctcc

SEQ ID NO: 482 - DNA, 17 bp, Artificial Sequence
  Forward primer 2 MYCL
gctgggcgaacccaaga

SEQ ID NO: 483 - DNA, 28 bp, Artificial Sequence
  Reverse primer 2 MYCL
tcttctctactgtcacaacatcaatttc

SEQ ID NO: 484 - DNA, 15 bp, Artificial Sequence
  Synthetic 2 MYCL
cgactcggagaatga

SEQ ID NO: 485 - DNA, 22 bp, Artificial Sequence
  Forward primer 1 CXCR1
gactgcagctcctactgttgga

SEQ ID NO: 486 - DNA, 25 bp, Artificial Sequence
  Reverse primer 1 CXCR1
ttcagcaatggtttgatctaactga

SEQ ID NO: 487 - DNA, 15 bp, Artificial Sequence
  Synthetic 1 CXCR1
acacctggccggtgc

SEQ ID NO: 488 - DNA, 17 bp, Artificial Sequence
  Forward primer 2 CXCR1
cctggccggtgcttcag

SEQ ID NO: 489 - DNA, 28 bp, Artificial Sequence
  Reverse primer 2 CXCR1
tctgtaatatttgacatgtcctcttcag

SEQ ID NO: 490 - DNA, 17 bp, Artificial Sequence
  Synthetic 2 CXCR1
agatcaaaccattgctg

SEQ ID NO: 491 - DNA, 20 bp, Artificial Sequence
  Forward primer 1 HCAR2
ttagggaaacggtggcagat

SEQ ID NO: 492 - DNA, 20 bp, Artificial Sequence
  Reverse primer 1 HCAR2
gattcctgcggtcacacctt

SEQ ID NO: 493 - DNA, 16 bp, Artificial Sequence
  Synthetic 1 HCAR2
agtgggagactggttg

SEQ ID NO: 494 - DNA, 23 bp, Artificial Sequence
  Forward primer 2 HCAR2
ttcacctacatgaacagcatgct

SEQ ID NO: 495 - DNA, 20 bp, Artificial Sequence
  Reverse primer 2 HCAR2
ggaaaggatgggctggagaa

SEQ ID NO: 496 - DNA, 16 bp, Artificial Sequence
  Synthetic 2 HCAR2
accccgtggtgtacta

SEQ ID NO: 497 - DNA, 15 bp, Artificial Sequence
  Forward primer 1 C1QA
gcggccaggcctcaa

SEQ ID NO: 498 - DNA, 20 bp, Artificial Sequence
  Reverse primer 1 C1QA
ccccctggtctcctttaagg

SEQ ID NO: 499 - DNA, 14 bp, Artificial Sequence
  Synthetic 1 C1QA
tccggacaggcatc

SEQ ID NO: 500 - DNA, 20 bp, Artificial Sequence
  Forward primer 1 C1QB
gcctcacaggacaccagctt

SEQ ID NO: 501 - DNA, 21 bp, Artificial Sequence
  Reverse primer 1 C1QB
cccatgggatcttcatcatca

SEQ ID NO: 502 - DNA, 15 bp, Artificial Sequence
  Synthetic 1 C1QB
ccaggaggcgtctga

SEQ ID NO: 503 - DNA, 19 bp, Artificial Sequence
  Reverse primer 2 C1QB
tgccccatgggatcttcat

SEQ ID NO: 504 - DNA, 18 bp, Artificial Sequence
  Forward primer 1 C1QC
tgtgccaggccagaaacc

SEQ ID NO: 505 - DNA, 19 bp, Artificial Sequence
  Reverse primer 1 C1QC
agcagcttcagcccaaggt

SEQ ID NO: 506 - DNA, 16 bp, Artificial Sequence
  Synthetic 1 C1QC
ccttctccgggatgga

SEQ ID NO: 507 - DNA, 25 bp, Artificial Sequence
  Forward primer 2 C1QC
gaagcagatctgaggacatctctgt

SEQ ID NO: 508 - DNA, 18 bp, Artificial Sequence
  Reverse primer 2 C1QC
cgggaatggctgggattc

SEQ ID NO: 509 - DNA, 13 bp, Artificial Sequence
  Synthetic 2 C1QC
ccgcccacctgca

SEQ ID NO: 510 - DNA, 20 bp, Artificial Sequence
  Forward primer 1 MRAS
caccagggagcaaggaaaag

SEQ ID NO: 511 - DNA, 24 bp, Artificial Sequence
  Reverse primer 1 MRAS
ggcactggtttctatgtacggaat

SEQ ID NO: 512 - DNA, 16 bp, Artificial Sequence
  Synthetic 1 MRAS
tggcgaccaaacacaa

SEQ ID NO: 513 - DNA, 21 bp, Artificial Sequence
  Forward primer 2 MRAS
caatgtcgacaaagccttcca

SEQ ID NO: 514 - DNA, 21 bp, Artificial Sequence
  Reverse primer 2 MRAS
ggcttttttccggaatctgtt

SEQ ID NO: 515 - DNA, 19 bp, Artificial Sequence
  Synthetic 2 MRAS
acctcgttagagtaattag

SEQ ID NO: 516 - DNA, 19 bp, Artificial Sequence
  Forward primer 1 SLC39A8
gcacttgctggaggcatgt

SEQ ID NO: 517 - DNA, 25 bp, Artificial Sequence
  Reverse primer 1 SLC39A8
tcagcatatcattcatctctggaaa

SEQ ID NO: 518 - DNA, 23 bp, Artificial Sequence
  Synthetic 1 SLC39A8
cctctatatttctctggcagata

SEQ ID NO: 519 - DNA, 25 bp, Artificial Sequence
  Forward primer 2 SLC39A8
gggactcagtacttccatagcaatc

SEQ ID NO: 520 - DNA, 25 bp, Artificial Sequence
  Reverse primer 2 SLC39A8
tgcattgagtaggatcacaaagtct

SEQ ID NO: 521 - DNA, 19 bp, Artificial Sequence
  Synthetic 2 SLC39A8
tgaggagtttccccacgag

SEQ ID NO: 522 - DNA, 16 bp, Artificial Sequence
  Forward primer 1 TMEM37
cgggcgcagcatgact

SEQ ID NO: 523 - DNA, 22 bp, Artificial Sequence
  Reverse primer 1 TMEM37
ccggatgaaggattcaaagaag

SEQ ID NO: 524 - DNA, 14 bp, Artificial Sequence
  Synthetic 1 TMEM37
ccgtcggcgtgcag

SEQ ID NO: 525 - DNA, 14 bp, Artificial Sequence
  Synthetic 2 TMEM37
cccagaggcctttg

SEQ ID NO: 526 - DNA, 22 bp, Artificial Sequence
  Forward primer 1 FCER1A
ggcagctggactatgagtctga

SEQ ID NO: 527 - DNA, 22 bp, Artificial Sequence
  Reverse primer 1 FCER1A
cttctcacgcggagcttttatt

SEQ ID NO: 528 - DNA, 16 bp, Artificial Sequence
  Synthetic 1 FCER1A
cccctcaacattactg

SEQ ID NO: 529 - DNA, 21 bp, Artificial Sequence
  Forward primer 2 FCER1A
accgagcatgggcctatattt

SEQ ID NO: 530 - DNA, 22 bp, Artificial Sequence
  Reverse primer 2 FCER1A
catggactcctggtgcttactg

SEQ ID NO: 531 - DNA, 17 bp, Artificial Sequence
  Synthetic 2 FCER1A
aagccttagatctctcc

SEQ ID NO: 532 - DNA, 18 bp, Artificial Sequence
  Forward primer 1 BCL11B
gcaacccgcagcacttgt

SEQ ID NO: 533 - DNA, 16 bp, Artificial Sequence
  Reverse primer 1 BCL11B
tggcggcctccacatg

SEQ ID NO: 534 - DNA, 15 bp, Artificial Sequence
  Synthetic 1 BCL11B
ccagagggagctcat

SEQ ID NO: 535 - DNA, 20 bp, Artificial Sequence
  Forward primer 2 BCL11B
tcccagagggagctcatcac

SEQ ID NO: 536 - DNA, 22 bp, Artificial Sequence
  Reverse primer 2 BCL11B
tctccagaccctcgtcttcttc

SEQ ID NO: 537 - DNA, 17 bp, Artificial Sequence
  Synthetic 2 BCL11B
aggctgaccatgtggag

SEQ ID NO: 538 - DNA, 22 bp, Artificial Sequence
  Forward primer 1 PKHD1
tgaggatctatgaacggctcaa

SEQ ID NO: 539 - DNA, 22 bp, Artificial Sequence
  Reverse primer 1 PKHD1
ccatcctccgtgacatgtacac

SEQ ID NO: 540 - DNA, 14 bp, Artificial Sequence
  Synthetic 1 PKHD1
caccggcatattgg

SEQ ID NO: 541 - DNA, 25 bp, Artificial Sequence
  Forward primer 2 PKHD1
cattacaacccaggaaagatttagg

SEQ ID NO: 542 - DNA, 26 bp, Artificial Sequence
  Reverse primer 2 PKHD1
gagatattttcttggacttgcacagt

SEQ ID NO: 543 - DNA, 21 bp, Artificial Sequence
  Synthetic 2 PKHD1
aaagtagtctgtcctgaatta

SEQ ID NO: 544 - DNA, 24 bp, Artificial Sequence
  Forward primer 1 KLRB1
ttggttgttactgggttgagtgtt

SEQ ID NO: 545 - DNA, 25 bp, Artificial Sequence
  Reverse primer 1 KLRB1
ccacactgcatttttctattgatga

SEQ ID NO: 546 - DNA, 20 bp, Artificial Sequence
  Synthetic 1 KLRB1
cagtgacatccttaatacag

SEQ ID NO: 547 - DNA, 25 bp, Artificial Sequence
  Forward primer 2 KLRB1
caacagagcaggaataaaacaacag

SEQ ID NO: 548 - DNA, 20 bp, Artificial Sequence
  Reverse primer 2 KLRB1
tctcggagttgctgccaata

SEQ ID NO: 549 - DNA, 15 bp, Artificial Sequence
  Synthetic 2 KLRB1
ccgggtctcttaaac

SEQ ID NO: 550 - DNA, 19 bp, Artificial Sequence
  Forward primer 1 LILRB5
ccagcccagttgctgacat

SEQ ID NO: 551 - DNA, 19 bp, Artificial Sequence
  Reverse primer 1 LILRB5
tgtccttcacggcagcatt

SEQ ID NO: 552 - DNA, 14 bp, Artificial Sequence
  Synthetic 1 LILRB5
caggaggaaattct

SEQ ID NO: 553 - DNA, 18 bp, Artificial Sequence
  Forward primer 2 LILRB5
ccagggcctgcagaagag

SEQ ID NO: 554 - DNA, 20 bp, Artificial Sequence
  Reverse primer 2 LILRB5
cggcagcattgagaatttcc

SEQ ID NO: 555 - DNA, 16 bp, Artificial Sequence
  Synthetic 2 LILRB5
ccagcccagttgctga

SEQ ID NO: 556 - DNA, 25 bp, Artificial Sequence
  Forward primer 1 NECAB1
tctacaatgctagttcctgcttcgt

SEQ ID NO: 557 - DNA, 25 bp, Artificial Sequence
  Reverse primer 1 NECAB1
gcacttggaaccataaagaaaatgt

SEQ ID NO: 558 - DNA, 19 bp, Artificial Sequence
  Synthetic 1 NECAB1
tcctgaacaactagatgtt

SEQ ID NO: 559 - DNA, 21 bp, Artificial Sequence
  Forward primer 2 NECAB1
acaacagcttctccccaaaca

SEQ ID NO: 560 - DNA, 21 bp, Artificial Sequence
  Reverse primer 2 NECAB1
ctgggtcatccactggttgtc

SEQ ID NO: 561 - DNA, 16 bp, Artificial Sequence
  Synthetic 2 NECAB1
atgtcagcggtccagg

SEQ ID NO: 562 - DNA, 18 bp, Artificial Sequence
  Forward primer 1 NECAB2
gtggaggccatcgaggaa

SEQ ID NO: 563 - DNA, 20 bp, Artificial Sequence
  Reverse primer 1 NECAB2
gctgtggctgggtttgatgt

SEQ ID NO: 564 - DNA, 15 bp, Artificial Sequence
  Synthetic 1 NECAB2
cagctccgacagaac

SEQ ID NO: 565 - DNA, 18 bp, Artificial Sequence
  Forward primer 2 NECAB2
gccgtgcggacaaaaatg

SEQ ID NO: 566 - DNA, 23 bp, Artificial Sequence
  Reverse primer 2 NECAB2
tctgcaaagaagagctggaattc

SEQ ID NO: 567 - DNA, 16 bp, Artificial Sequence
  Synthetic 2 NECAB2
tgatgggaagctgtcc

SEQ ID NO: 568 - DNA, 20 bp, Artificial Sequence
  Forward primer 1 ALAS1
gatgatgccaggctgtgaga

SEQ ID NO: 569 - DNA, 20 bp, Artificial Sequence
  Reverse primer 1 ALAS1
cgaatcccttggatcatgga

SEQ ID NO: 570 - DNA, 17 bp, Artificial Sequence
  Synthetic 1 ALAS1
ctgattctgggaaccat

SEQ ID NO: 571 - DNA, 21 bp, Artificial Sequence
  Forward primer 2 ALAS1
aaccctcttcaccctggctaa

SEQ ID NO: 572 - DNA, 20 bp, Artificial Sequence
  Reverse primer 2 ALAS1
ggcatggttcccagaatcag

SEQ ID NO: 573 - DNA, 16 bp, Artificial Sequence
  Synthetic 2 ALAS1
atgatgccaggctgtg

SEQ ID NO: 574 - DNA, 19 bp, Artificial Sequence
  Forward primer 1 HMBS
cctgcccactgtgcttcct

SEQ ID NO: 575 - DNA, 19 bp, Artificial Sequence
  Reverse primer 1 HMBS
ggttttcccgcttgcagat

SEQ ID NO: 576 - DNA, 15 bp, Artificial Sequence
  Synthetic 1 HMBS
ctggcttcaccatcg

SEQ ID NO: 577 - DNA, 22 bp, Artificial Sequence
  Forward primer 2 HMBS
gcctgtttaccaaggagcttga

SEQ ID NO: 578 - DNA, 22 bp, Artificial Sequence
  Reverse primer 2 HMBS
tgaacaaccaggtccacttcat

SEQ ID NO: 579 - DNA, 15 bp, Artificial Sequence
  Synthetic 2 HMBS
catgccctggagaag

SEQ ID NO: 580 - DNA, 18 bp, Artificial Sequence
  Forward primer 1 GTF2D1 (TBP)
gcacttcgtgcccgaaac

SEQ ID NO: 581 - DNA, 20 bp, Artificial Sequence
  Reverse primer 1 GTF2D1 (TBP)
cctcatgattaccgcagcaa

SEQ ID NO: 582 - DNA, 19 bp, Artificial Sequence
  Synthetic 1 GTF2D1 (TBP)
cgaatataatcccaagcgg

SEQ ID NO: 583 - DNA, 18 bp, Artificial Sequence
  Forward primer 2 GTF2D1 (TBP)
gcccgaaacgccgaatat

SEQ ID NO: 584 - DNA, 22 bp, Artificial Sequence
  Reverse primer 2 GTF2D1 (TBP)
cgtggctctcttatcctcatga

SEQ ID NO: 585 - DNA, 16 bp, Artificial Sequence
  Synthetic 2 GTF2D1 (TBP)
agcggtttgctgcggt

SEQ ID NO: 586 - DNA, 2458 bp, Homo sapiens
cagaagaaggcagcgcccaaggcgcatgcgcagcggtcactcccgctgtatattaaggcgccggcgatcgcggcctgaggctgctcccggacaagggcaacgagcgtttcgtttggacttctcgacttgagtgcccgcctccttcgccgccgcctctgcagtcctcagcgcagttatgcccagttcttcccgctgtggggacacgaccacggaggaatccttgcttcagggactcgggaccctgctggaccccttcctcgggtttaggggatgtggggaccaggagaaagtcaggatccctaagagtcttccctgcctggatggatgagtggcttcttctccacctagattctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtgaaaaaa

SEQ ID NO: 587 - DNA, 2281 bp, Homo sapiens
cagaagaaggcagcgcccaaggcgcatgcgcagcggtcactcccgctgtatattaaggcgccggcgatcgcggcctgaggctgctcccggacaagggcaacgagcgtttcgtttggacttctcgacttgagtgcccgcctccttcgccgccgcctctgcagtcctcagcgcagtctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtgaaaaaa

SEQ ID NO: 588 - DNA, 1921 bp, Homo sapiens
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 589 - DNA, 1526 bp, Homo sapiens
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

(19) United States
(12) Patent Application Publication
(10) Pub. No.: US 2020/0131574 A1
(43) Pub. Date: Apr. 30, 2020

(54) ASSAY FOR DISTINGUISHING BETWEEN SEPSIS AND SYSTEMIC INFLAMMATORY RESPONSE SYNDROME

(71) Applicants: THE SECRETARY OF STATE FOR HEALTH, London, Greater London (GB); UNIVERSITY COLLEGE CARDIFF CONSULTANTS LIMITED, Cardiff, South Glamorgan (GB)

(72) Inventors: Judith HALL, Cardiff (GB); Tamas SZAKMANY, Cardiff (GB); Sanjoy SHAH, Bristol (GB); Karen KEMPSELL, Salisbury (GB); Graham BALL, Nottingham (GB)

(73) Assignees: The Secretary of State for Health, London, Greater London (GB); University College Cardiff Consultants Limited, Cardiff, South Glamorgan (GB)

(21) Appl. No.: 16/335,830

(22) PCT Filed: Sep. 29, 2017
(86) PCT No.: PCT/GB2017/052945

§ 371 Date: Mar. 22, 2019

(30) Foreign Application Priority Data
Sep. 29, 2016 (GB) 1616557.3

(51) Int. Cl. C12Q 1/6883; G01N 33/564

(52) U.S. Cl. CPC C12Q 1/6883; G01N 33/564; C12Q 2600/112; G01N 2800/24; C12Q 2600/158; G01N 2800/26

(57) ABSTRACT

There is provided a method for distinguishing between sepsis and systemic inflammatory response syndrome (SIRS) in a patient, comprising: (i) determining the amount of one or more biomarker for sepsis, and one or more biomarker for SIRS in a sample obtained from a patient, wherein the one or more biomarker for sepsis is selected from the group consisting of: ITGB3, ITGA2B, MYL9, LCN2, TREML1, LCN15, CMTMS, PPBP, and PF4; and the one or more biomarker for SIRS is selected from the group consisting of: PLA2G7, ARHGEF10L, MYCL, TGFBI, and GPR124, (ii) comparing the amount of the one or more biomarker for sepsis determined in said sample in (i) to a corresponding reference value representative of a healthy individual, (iii) comparing the amount of the one or more biomarker for SIRS determined in said sample in (i) to a corresponding reference value representative of a healthy individual; wherein the patient is diagnosed as having sepsis, when an increase is observed in the one or more biomarker for sepsis, and no increase is observed in the one or more biomarker for SIRS, in the sample obtained from the patient relative to the corresponding reference value; and wherein the patient is diagnosed as having SIRS, when an increase is observed in the one or more biomarker for SIRS, and no increase is observed in the one or more biomarker for sepsis, in the sample obtained from the patient relative to the corresponding reference value.

### CROSS-REFERENCES TO RELATED APPLICATIONS

This application is the National Stage of International Application No. PCT/GB2017/052945, filed Sep. 29, 2017, which claims priority to Great Britain Application No. 1616557.3, filed Sep. 29, 2016, the disclosures of which are hereby incorporated by reference in their entirety.

### STATEMENT REGARDING SEQUENCE LISTING

The sequence listing associated with this application is provided in text format in lieu of a paper copy and is hereby incorporated by reference into the specification. The name of the text file containing the sequence listing is 68584_Seq_Rev_Final_2019-12-16.txt. The text file is 431 KB; was created on Dec. 16, 2019, contains no new matter, and is being submitted via EFS-Web.

The present invention relates to one or more biomarkers associated with systemic inflammatory conditions, such as Severe Inflammatory Response Syndrome (SIRS) and sepsis. More particularly, the invention relates to methods for diagnosing, monitoring and prognosing systemic inflammatory conditions, such as Severe Inflammatory Response Syndrome (SIRS), sepsis, abdominal sepsis and pulmonary sepsis, and for distinguishing between sepsis and SIRS in a patient.

Systemic inflammatory conditions such as Severe Inflammatory Response Syndrome (SIRS) and sepsis are life-threatening conditions that can result in organ failure and death.

Sepsis (or blood poisoning) is characterised by a systemic host response to infection. Sepsis affects approximately 25% of intensive care patients, and is estimated to cause over 37,000 deaths in the UK every year, with a mortality rate of between 28% and 50%. Diagnosis of sepsis is typically performed using culture-based methods, involving microbial growth followed by taxonomic identification of the pathogen. However, these culture-based techniques are time-consuming, taking over 24 hours to obtain results, and have poor sensitivity and specificity. Other more recently developed diagnostic methods involve assessment of single blood protein biomarkers such as CRP and pro-calcitonin. These methods allow quicker diagnosis, but there is growing evidence that these markers suffer from poor specificity. Furthermore, none of the available methods provide an insight into the underlying origin or aetiology of the disease, nor do they provide any way of predicting recovery from sepsis or the likelihood of progression to severe sepsis or septic shock. Treatment of sepsis typically involves administration of antimicrobials such as broad-spectrum antibiotics together with intravenous fluids. It is estimated that mortality increases by approximately 5% for every hour that treatment is delayed. Rapid diagnosis and initiation of treatment of patients having sepsis is therefore essential.

Severe Inflammatory Response Syndrome (SIRS) is also characterised by a systemic host response. However, this condition does not result from infection, but instead results from injury or trauma. Clinical symptoms of SIRS are similar to sepsis, and thus it can be difficult to distinguish between patients in the early stages of sepsis and patients who have infection-negative systemic inflammation (SIRS). Making an incorrect distinction between these two conditions has both clinical and economic implications, including inappropriate patient management, and unnecessary over-prescription of antibiotics. There is currently no clinical test available to distinguish between sepsis and non-infective SIRS. Development of a method for effectively stratifying SIRS and sepsis patients would therefore be beneficial, allowing sepsis patients to receive early, effective and aggressive treatment with antimicrobials and supportive care, and reducing the unnecessary exposure of SIRS patients to antibiotics.

There is therefore a need to provide improved ways to diagnose, monitor and/or prognose patients that have or are at risk of developing systemic inflammatory conditions (such as sepsis and SIRS) in order to facilitate early intervention and appropriate treatment. In particular, there is a need to provide for effective ways of distinguishing between sepsis and SIRS in patients in order to ensure that an appropriate treatment regimen is selected.

### SUMMARY OF THE INVENTION

By conducting extensive investigations into expression patterns associated with systemic inflammatory conditions, the present inventors have identified biomarkers that may be used to evaluate various aspects of systemic inflammatory conditions, such as SIRS and sepsis. The biomarkers may be used to diagnose the presence (or absence) of a systemic inflammatory condition, and to distinguish between different types of systemic inflammatory conditions in a patient. The biomarkers may also be used to monitor a patient having a systemic inflammatory condition, and to determine whether a patient is suitable for discharge from medical care. The present invention therefore provides a solution to one or more of the above mentioned problems.

In one aspect, the present invention provides a method for distinguishing between sepsis and systemic inflammatory response syndrome (SIRS) in a patient, comprising:

(i) determining the amount of one or more biomarker for sepsis, and one or more biomarker for SIRS in a sample obtained from a patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124,
(ii) comparing the amount of the one or more biomarker for sepsis determined in said sample in (i) to a corresponding reference value representative of a healthy individual,
(iii) comparing the amount of the one or more biomarker for SIRS determined in said sample in (i) to a corresponding reference value representative of a healthy individual;
wherein the patient is diagnosed as having sepsis, when an increase is observed in the one or more biomarker for sepsis, and no increase is observed in the one or more biomarker for SIRS, in the sample obtained from the patient relative to the corresponding reference value; and wherein the patient is diagnosed as having SIRS, when an increase is observed in the one or more biomarker for SIRS, and no increase is observed in the one or more biomarker for sepsis, in the sample obtained from the patient relative to the corresponding reference value.

In a related aspect, the present invention provides a method for distinguishing between sepsis and systemic inflammatory response syndrome (SIRS) in a patient, comprising:

(a) diagnosing a patient as having a systemic inflammatory condition by performing a method comprising:
(i) determining the amount of one or more biomarker in a sample obtained from the patient, wherein the one or more biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1,
(ii) comparing the amount of the one or more biomarker determined in said sample in (i) to a corresponding reference value to determine that the patient has a systemic inflammatory condition; and
   (b) determining whether the patient diagnosed as having a systemic inflammatory condition has sepsis or SIRS by performing a method comprising:
(i) determining the amount of one or more biomarker for sepsis, and one or more biomarker for SIRS in a sample obtained from the patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124,
(ii) comparing the amount of the one or more biomarker for sepsis determined in said sample in (i) to a corresponding reference value representative of a healthy individual,
(iii) comparing the amount of the one or more biomarker for SIRS determined in said sample in (i) to a corresponding reference value representative of a healthy individual;
wherein the patient is diagnosed as having sepsis, when an increase is observed in the one or more biomarker for sepsis, and no increase is observed in the one or more biomarker for SIRS, in the sample obtained from the patient relative to the corresponding reference value; and wherein the patient is diagnosed as having SIRS, when an increase is observed in the one or more biomarker for SIRS, and no increase is observed in the one or more biomarker for sepsis, in the sample obtained from the patient relative to the corresponding reference value.

In a related aspect, the present invention provides a method for distinguishing between sepsis and systemic inflammatory response syndrome (SIRS) in a patient diagnosed as having a systemic inflammatory condition, comprising:

(i) determining the amount of one or more biomarker for sepsis in a sample obtained from a patient, wherein the one or more biomarker for sepsis is selected from the group consisting of: ITGB3, ITGA2B, MYL9, LCN2, TREML1, LCN15, CMTM5, PPBP, and PF4,
(ii) comparing the amount of the one or more biomarker for sepsis determined in said sample in (i) to a corresponding reference value representative of a healthy individual;
wherein the patient is diagnosed as having sepsis, when an increase is observed in the one or more biomarker for sepsis in the sample obtained from the patient relative to the corresponding reference value; and wherein the patient is diagnosed as having SIRS, when no increase is observed in the one or more biomarker for sepsis, in the sample obtained from the patient relative to the corresponding reference value.

In a related aspect, the present invention provides a method for distinguishing between sepsis and systemic inflammatory response syndrome (SIRS) in a patient diagnosed as having a systemic inflammatory condition, comprising:

(i) determining the amount of one or more biomarker for SIRS in a sample obtained from a patient, wherein the one or more biomarker for SIRS is selected from the group consisting of: PLA2G7, ARHGEF10L, MYCL, TGFBI, and GPR124,
(ii) comparing the amount of the one or more biomarker for SIRS determined in said sample in (i) to a corresponding reference value representative of a healthy individual;
wherein the patient is diagnosed as having SIRS, when an increase is observed in the one or more biomarker for SIRS in the sample obtained from the patient relative to the corresponding reference value; and wherein the patient is diagnosed as having sepsis, when no increase or a decrease is observed in the one or more biomarker for SIRS, in the sample obtained from the patient relative to the corresponding reference value.

In a further related aspect, the present invention provides the use of one or more of: ITGB3, ITGA2B, MYL9, LCN2, TREML1, LCN15, CMTM5, PPBP, and PF4 and/or one or more of: PLA2G7, ARHGEF10L, MYCL, TGFBI, and GPR124 for distinguishing between sepsis and SIRS in a patient.

In a further aspect, the present invention provides a method for diagnosing whether a patient has a systemic inflammatory condition, comprising:

(i) determining the amount of FAM20A and OLAH in a sample obtained from a patient,
(ii) comparing the amount of FAM20A determined in said sample in (i) to a corresponding reference value representative of a healthy individual,
(iii) comparing the amount of OLAH determined in said sample in (i) to a corresponding reference value representative of a healthy individual;
wherein the patient is diagnosed as having a systemic inflammatory condition, when an increase is observed in FAM20A and OLAH in the sample obtained from the patient relative to the corresponding reference value; and wherein the patient is diagnosed as not having a systemic inflammatory condition, when no increase is observed in FAM20A and OLAH, in the sample obtained from the patient relative to the corresponding reference value.

In a further related aspect, the present invention provides the use of FAM20A and OLAH for diagnosing a systemic inflammatory condition in a patient.

In a further aspect, the present invention provides a method for diagnosing whether a patient has abdominal sepsis, comprising:

(i) determining the amount of one or more biomarker in a sample obtained from a patient, wherein the one or more biomarker is selected from the group consisting of: SLC39A8, CIQC, CIQA, MRAS, TMEM37, CIQB, PCOLCE2, KIF2C, TNF, IF144, IFIT1, and RPGRIP1,
(ii) comparing the amount of the one or more biomarker determined in said sample in (i) to a corresponding reference value; and thereby determining whether the patient has abdominal sepsis.

In a related aspect, the present invention provides a method for diagnosing whether a patient has abdominal sepsis, comprising:
(a) diagnosing a patient as having sepsis by performing a method to distinguish between sepsis and SIRS in the patient, comprising:
   (i) determining the amount of one or more biomarker for sepsis, and one or more biomarker for SIRS in a sample obtained from the patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124,
   (ii) comparing the amount of the one or more biomarker for sepsis determined in said sample in (i) to a corresponding reference value representative of a healthy individual,
   (iii) comparing the amount of the one or more biomarker for SIRS determined in said sample in (i) to a corresponding reference value representative of a healthy individual; wherein the patient is diagnosed as having sepsis, when an increase is observed in the one or more biomarker for sepsis, and no increase is observed in the one or more biomarker for SIRS, in the sample obtained from the patient relative to the corresponding reference value; and
(b) determining whether the patient diagnosed as having sepsis has abdominal sepsis by performing a method comprising:
   (i) determining the amount of one or more biomarker in a sample obtained from the patient, wherein the one or more biomarker is selected from the group consisting of: SLC39A8, CIQC, CIQA, MRAS, TMEM37, CIQB, PCOLCE2, KIF2C, TNF, IF144, IFIT1, and RPGRIP1,
   (ii) comparing the amount of the one or more biomarker determined in said sample in (i) to a corresponding reference value; and thereby determining whether the patient has abdominal sepsis.

In a related aspect, the present invention provides a method for diagnosing whether a patient has abdominal sepsis, comprising:
(a) diagnosing a patient as having a systemic inflammatory condition by performing a method comprising:
   (i) determining the amount of one or more biomarker in a sample obtained from the patient, wherein the one or more biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1,
   (ii) comparing the amount of the one or more biomarker determined in said sample in (i) to a corresponding reference value to determine that the patient has a systemic inflammatory condition;
(b) determining that the patient diagnosed as having a systemic inflammatory condition has sepsis by performing a method to distinguish between sepsis and SIRS in the patient, comprising:
   (i) determining the amount of one or more biomarker for sepsis, and one or more biomarker for SIRS in a sample obtained from the patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124,
   (ii) comparing the amount of the one or more biomarker for sepsis determined in said sample in (i) to a corresponding reference value representative of a healthy individual,
   (iii) comparing the amount of the one or more biomarker for SIRS determined in said sample in (i) to a corresponding reference value representative of a healthy individual; wherein the patient is diagnosed as having sepsis, when an increase is observed in the one or more biomarker for sepsis, and no increase is observed in the one or more biomarker for SIRS, in the sample obtained from the patient relative to the corresponding reference value; and
(c) determining whether the patient diagnosed as having sepsis has abdominal sepsis by performing a method comprising:
   (i) determining the amount of one or more biomarker in a sample obtained from a patient, wherein the one or more biomarker is selected from the group consisting of: SLC39A8, CIQC, CIQA, MRAS, TMEM37, CIQB, PCOLCE2, KIF2C, TNF, IF144, IFIT1, and RPGRIP1,
   (ii) comparing the amount of the one or more biomarker determined in said sample in (i) to a corresponding reference value; and thereby determining whether the patient has abdominal sepsis.

In a related aspect, the present invention provides the use of one or more of: SLC39A8, CIQC, CIQA, MRAS, TMEM37, CIQB, PCOLCE2, KIF2C, TNF, IF144, IFIT1, and RPGRIP1 as a biomarker of abdominal sepsis in a patient.

In a further aspect, the present invention provides a method for diagnosing whether a patient has pulmonary sepsis, comprising:

(i) determining the amount of one or more biomarker in a sample obtained from a patient, wherein the one or more biomarker is selected from the group consisting of: HCAR2, CXCR1, DISC1, EPSTI1, and IF144,
(ii) comparing the amount of the one or more biomarker determined in said sample in (i) to a corresponding reference value; and thereby determining whether the patient has pulmonary sepsis.

In a related aspect, the present invention provides a method for diagnosing whether a patient has pulmonary sepsis, comprising:
(a) diagnosing a patient as having sepsis by performing a method to distinguish between sepsis and SIRS in the patient, comprising:
   (i) determining the amount of one or more biomarker for sepsis, and one or more biomarker for SIRS in a sample obtained from the patient, wherein the one or more biomarker for sepsis is selected from the group consisting of: ITGB3, ITGA2B, MYL9, LCN2, TREML1, LCN15, CMTM5, PPBP, and PF4 and the one or more biomarker for SIRS is selected from the group consisting of: PLA2G7, ARHGEF10L, MYCL, TGFBI, and GPR124L,
   (ii) comparing the amount of the one or more biomarker for sepsis determined in said sample in (i) to a corresponding reference value representative of a healthy individual,
   (iii) comparing the amount of the one or more biomarker for SIRS determined in said sample in (i) to a corresponding reference value representative of a healthy individual; wherein the patient is diagnosed as having sepsis, when an increase is observed in the one or more biomarker for sepsis, and no increase is observed in the one or more biomarker for SIRS, in the sample obtained from the patient relative to the corresponding reference value;
(b) determining whether the patient diagnosed as having sepsis has pulmonary sepsis by performing a method comprising:
   (i) determining the amount of one or more biomarker in a sample obtained from the patient, wherein the one or more biomarker is selected from the group consisting of: HCAR2, CXCR1, DISC1, EPSTI1, and IF144,
   (ii) comparing the amount of the one or more biomarker determined in said sample in (i) to a corresponding reference value; and thereby determining whether the patient has pulmonary sepsis.

In a further related aspect, the present invention provides a method for diagnosing whether a patient has pulmonary sepsis, comprising:
(a) diagnosing a patient as having a systemic inflammatory condition, by performing a method comprising:
   (i) determining the amount of one or more biomarker in a sample obtained from the patient, wherein the one or more biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1,
   (ii) comparing the amount of the one or more biomarker determined in said sample in (i) to a corresponding reference value to determine that the patient has a systemic inflammatory condition;
(b) determining that the patient diagnosed as having a systemic inflammatory condition has sepsis by performing a method to distinguish between sepsis and SIRS in the patient, comprising:
   (i) determining the amount of one or more biomarker for sepsis, and one or more biomarker for SIRS in a sample obtained from the patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124,
   (ii) comparing the amount of the one or more biomarker for sepsis determined in said sample in (i) to a corresponding reference value representative of a healthy individual,
   (iii) comparing the amount of the one or more biomarker for SIRS determined in said sample in (i) to a corresponding reference value representative of a healthy individual; wherein the patient is diagnosed as having sepsis, when an increase is observed in the one or more biomarker for sepsis, and no increase is observed in the one or more biomarker for SIRS, in the sample obtained from the patient relative to the corresponding reference value;
(c) determining whether the patient diagnosed as having sepsis has pulmonary sepsis by performing a method comprising:
   (i) determining the amount of one or more biomarker in a sample obtained from the patient, wherein the one or more biomarker is selected from the group consisting of: HCAR2, CXCR1, DISC1, EPSTI1, and IF144,
   (ii) comparing the amount of the one or more biomarker determined in said sample in (i) to a corresponding reference value; and thereby determining whether the patient has pulmonary sepsis.

In a related aspect, the present invention provides the use of one or more of: HCAR2, CXCR1, DISC1, EPSTI1, and IF144 as a biomarker of pulmonary sepsis in a patient.

In a further aspect, the present invention provides a method for monitoring a systemic inflammatory condition in a patient, comprising:
(i) determining the amount of one or more biomarker in a sample obtained from a patient at a first (or earlier) time point;
(ii) determining the amount of the one or more biomarker in a sample obtained from the patient at one or more later time points;
(iii) comparing the amount of the one or more biomarker determined in step (ii) to the amount of the one or more biomarker determined in step (i);
   wherein the one or more biomarker is selected from the group consisting of: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1, and LILRB5.

In one embodiment, the method is for monitoring a patient having abdominal sepsis, and comprises the steps of:

(i) determining the amount of one or more biomarker in a sample obtained from a patient having abdominal sepsis at a first time point;
(ii) determining the amount of the one or more biomarker in a sample obtained from the patient at one or more later time points;
(iii) comparing the amount of the one or more biomarker determined in step (ii) to the amount of the one or more biomarker determined in step (i);
wherein the one or more biomarker is selected from the group consisting of: ITM2A, CCL5, NPPC, KLRK1, KLRB1, HLA-DRA, BCL11B, HLA-DPB1, SLAMF6, CD160, KLRF1, and MX1.

In one embodiment, the method is for monitoring a patient having SIRS, and comprises the steps of:

(i) determining the amount of one or more biomarker in a sample obtained from a patient having SIRS at a first time point;
(ii) determining the amount of the one or more biomarker in a sample obtained from the patient at one or more later time points;
(iii) comparing the amount of the one or more biomarker determined in step (ii) to the amount of the one or more biomarker determined in step (i);
wherein the one or more biomarker is selected from the group consisting of: CCL5, NPPC, PKD1, LGALS2, MYCL, NECAB1, and PKHD1.

In a related aspect, the present invention provides the use of one or more of: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1 and LILRB5, as a biomarker for monitoring a patient having a systemic inflammatory condition.

In a related aspect, the invention provides a method for determining whether a patient having a systemic inflammatory condition is suitable for discharge from medical care, comprising:

(i) determining the amount of one or more biomarker selected from the group consisting of: NECAB1, NECAB2, PKD1, PKHD1, LILRB4, and LILRB5 in a sample obtained from a patient,
(ii) comparing the amount of the one or more biomarker determined in said sample in (i) to a corresponding reference value, and thereby determining whether the patient is suitable for discharge from medical care.

In one embodiment, the method is for determining whether a patient being treated for SIRS is suitable for discharge from medical care, and comprises the steps of:
(i) determining the amount of one or more biomarker selected from: NECAB1, PKDI, PKHD1, LILRB4, and LILRB5 in a sample obtained from a patient being treated for SIRS,
(ii) comparing the amount of one or more biomarker determined in said sample in (i) to a corresponding reference value; and thereby determining whether the patient is suitable for discharge from medical care.

In one embodiment, the method is for determining whether a patient being treated for sepsis is suitable for discharge from medical care, and comprises the steps of:

(i) determining the amount one or more biomarker selected from: NECAB2, PKD1, PKHD1 and LILRB5 in a sample obtained from a patient being treated for sepsis,
(ii) comparing the amount of the one or more biomarker determined in said sample in (i) to a corresponding reference value; and thereby determining whether the patient is suitable for discharge from medical care.

In a related aspect, the present invention provides the use of one or more of: NECAB1, NECAB2, PKD1, PKHD1, LILRB4, and LILRB5, for determining whether a patient having a systemic inflammatory condition is suitable for discharge from medical care.

In a further aspect, the present invention provides a device for carrying out the methods and uses of the invention. In one embodiment, the device comprises one or more binding agent specific for the one or more biomarker.

### DETAILED DESCRIPTION OF THE INVENTION

As described herein, the present inventors have conducted a temporal differential gene expression study in peripheral blood leukocytes (PBLs) in patients having SIRS, abdominal sepsis and pulmonary sepsis, and in normal healthy individuals. Using this method, the inventors have identified host biomarkers associated with different systemic inflammatory conditions. In particular, the present inventors have identified biomarkers that are elevated in patients having systemic inflammatory conditions, and can thus be used for diagnosis, monitoring and/or prognosis of these conditions. The present inventors have further identified biomarkers that are differentially regulated in different types of systemic inflammatory condition (e.g., SIRS and sepsis) and biomarkers that are differentially regulated in different types of sepsis (e.g., in abdominal sepsis and pulmonary sepsis). These biomarkers can therefore be used to specifically diagnose SIRS and sepsis (e.g., abdominal sepsis and pulmonary sepsis), and can also be used to distinguish between SIRS and sepsis, and/or between abdominal sepsis and pulmonary sepsis. These biomarkers may also be used to monitor a systemic inflammatory condition in a patient (e.g., to monitor the recovery of a patient). The present inventors have also identified biomarkers that are differentially regulated in patients that recover from a systemic inflammatory condition, and those that do not recover form a systemic inflammatory condition, and can thus be used to determine whether a patient is suitable for discharge from medical care. The new biomarkers for the systemic inflammatory conditions are listed in Tables 1-4 herein (together with corresponding sequence identifiers (SEQ ID NOs)). Tables 1-4 provide the HGNC gene IDs for the biomarkers of the invention. As would be understood by a person skilled in the art, the HGNC gene ID information can be used to determine the sequence of all the RNA transcripts, and thus all of the proteins which correspond to the biomarkers of the invention. Accession numbers for each of the biomarkers are also provided in the “Sequence Information” Section of the description.

Based on these findings, the present inventors have thus developed methods and uses that allow for rapid, sensitive and accurate diagnosis, monitoring and/or prognosis of systemic inflammatory conditions (such as sepsis and/or SIRS) using one or more biological samples obtained from a patient at a single time point, or during the course of disease progression. The inventors have also developed methods and uses that allow for different systemic inflammatory conditions (such as sepsis and/or SIRS) to reliably distinguished allowing for appropriate therapeutic intervention.

### Diagnosis of a Systemic Inflammatory Condition

As illustrated by Example 1 and FIG. 1, the present inventors observed that the levels of FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1, are elevated in patients having systemic inflammatory conditions (see Table 1), and can thus be used as biomarkers for diagnosis of systemic inflammatory conditions.

The present invention therefore provides a method for diagnosing a systemic inflammatory condition in a patient, comprising:

(i) determining the presence and/or amount of one or more inflammation biomarker in a sample obtained from a patient, wherein the one or more biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1;
(ii) comparing the presence and/or amount of the one or more inflammation biomarker determined in said sample in (i) to a corresponding reference value; and thereby determining whether the patient has or is at risk of developing a systemic inflammatory condition.

As used herein, the terms “diagnosis”, “diagnosing” and “diagnose(d)” refer to the process or act of recognising, deciding on or concluding on a disease or condition in a patient on the basis of symptoms and signs and/or from results of various diagnostic procedures (such as for example, from knowing the presence, absence or quantity of one or more biomarkers characteristic of the diagnosed disease or condition). In one embodiment, diagnosis of a systemic inflammatory condition in a patient comprises determining whether the patient has or is at risk of developing a systemic inflammatory condition.

As used herein, the term “systemic inflammatory condition” refers to a disease or condition comprising a systemic inflammatory response. In one embodiment, the term encompasses SIRS and sepsis. In one embodiment, the systemic inflammatory condition is one or more of SIRS and sepsis. In one embodiment, the systemic inflammatory condition is one or more of SIRS, abdominal sepsis and pulmonary sepsis.

As used herein, the term “systemic inflammatory response syndrome (SIRS)” refers to a systemic inflammatory response syndrome with no signs of infection. This condition may also be referred to as “non-infective SIRS” or “infection-free SIRS”. SIRS may be characterised by the presence of at least two of the four following clinical symptoms: fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCC >2 less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms (immature neutrophils).

As used herein, the term “sepsis” refers to the systemic inflammatory condition that occurs as a result of infection. Defined focus of infection is indicated by either (i) an organism grown in blood or sterile site; or (ii) an abscess or infected tissue (e.g., pneumonia, peritonitis, urinary tract, vascular line infection, soft tissue). In one embodiment, the infection may be a bacterial infection. The presence of sepsis is also characterised by the presence of at least two (of the four) systemic inflammatory response syndrome (SIRS) criteria defined above.

Sepsis may be characterised as mild sepsis, severe sepsis (sepsis with acute organ dysfunction), septic shock (sepsis with refractory arterial hypotension), organ failure, multiple organ dysfunction syndrome and death.

“Mild sepsis” can be defined as the presence of sepsis without organ dysfunction.

“Severe sepsis” can be defined as the presence of sepsis and at least one of the following manifestations of organ hypoperfusion or dysfunction: hypoxemia, metabolic acidosis, oliguria, lactic acidosis, or an acute alteration in mental status without sedation. Organ hypoperfusion or dysfunction is defined as a Sequential Organ Failure Assessment (SOFA) score ≥2 for the organ in question.

“Septic shock” can be defined as the presence of sepsis accompanied by a sustained decrease in systolic blood pressure (90 mm Hg or less, or a drop of at least 40 mm Hg from baseline systolic blood pressure) despite fluid resuscitation, and the need for vasoactive amines to maintain adequate blood pressure.

The term “sepsis” may include one or more of abdominal sepsis and pulmonary sepsis.

As used herein, the term “abdominal sepsis” refers to severe bacterial infection in the abdominal cavity (for example, but not restricted to perforated small and large bowel, pyelonephritis, spontaneous bacterial peritonitis, abscess in the peritoneal cavity, infection of the retroperitoneal space, infection in the liver, kidneys, pancreas, spleen); causing organ dysfunction. Organ hypoperfusion or dysfunction is defined as a Sequential Organ Failure Assessment (SOFA) score ≥2 for the organ in question.

As used herein, the term “pulmonary sepsis” refers to severe bacterial infection in the thoracic cavity, primarily affecting the lung and pleural space (for example, but not restricted to pneumonia, lung abscess, empyaema, mediastinitis, tracheobronchitis); causing organ dysfunction. Organ hypoperfusion or dysfunction is defined as a Sequential Organ Failure Assessment (SOFA) score ≥2 for the organ in question.

The terms “patient”, “individual”, and “subject”, are used interchangeably herein to refer to a mammalian subject for whom diagnosis, monitoring, prognosis, and/or treatment is desired. The mammal can be a human, non-human primate, mouse, rat, dog, cat, horse or cow, but is not limited to these examples. In one preferred embodiment, the individual, subject, or patient is a human, e.g., a male or female.

In one embodiment, the patient is suspected of having or being at risk of developing a systemic inflammatory condition (such as SIRS, sepsis, abdominal sepsis or pulmonary sepsis). For example, the patient may be a critically ill patient, e.g., a patient admitted to an intensive care unit (ICU) or emergency department (ED), in whom the incidence of SIRS and sepsis is known to be elevated. The patient may be admitted to ICU or ED with one or more of: serious trauma, chronic obstructive pulmonary disease (COPD), patients having undergone surgery, complications from surgery, medical shock, bacterial, fungal or viral infections, Acute Respiratory Distress Syndrome (ARDS), pulmonary and systemic inflammation, pulmonary tissue injury, severe pneumonia, respiratory failure, acute respiratory failure, respiratory distress, subarachnoidal hemorrhage (SAH), (severe) stroke, asphyxia, neurological conditions, organ dysfunction, single or multi-organ failure (MOF), poisoning and intoxication, severe allergic reactions and anaphylaxis, burn injury, acute cerebral hemorrhage or infarction, and any condition for which the patient requires assisted ventilation.

In one embodiment, the patient has been previously diagnosed as having or being at risk of developing a systemic inflammatory condition (eg. SIRS, sepsis, abdominal sepsis or pulmonary sepsis). In one embodiment, the patient may have been previously diagnosed as having or being at risk of developing a systemic inflammatory condition (eg. SIRS, sepsis, abdominal sepsis or pulmonary sepsis) using any of the methods described herein, or any combination of methods described herein.

In one embodiment, the patient has not been previously diagnosed as having a systemic inflammatory condition (eg. SIRS, sepsis, abdominal sepsis or pulmonary sepsis).

As used herein, the term “sample” encompasses any suitable biological material, for example blood, plasma, saliva, serum, sputum, urine, cerebral spinal fluid, cells, a cellular extract, a tissue sample, a tissue biopsy, a stool sample and the like. Furthermore, pools or mixtures of the above-mentioned samples may be employed. Typically, the sample is blood sample. The precise biological sample that is taken from the individual may vary, but the sampling preferably is minimally invasive and is easily performed by conventional techniques. In a preferred embodiment, the sample is a whole blood sample, a purified peripheral blood leukocyte sample or a cell type sorted leukocyte sample, such as a sample of the individual's neutrophils.

The methods and uses of the present invention may utilise samples that have undergone minimal or zero processing before testing. They may also utilise samples that have been manipulated, in any way, after procurement, such as treatment with reagents, solubilisation, or enrichment for certain components.

The methods of the present invention are in vitro methods. Thus, the methods of the present invention can be carried out in vitro on an isolated sample that has been obtained from a patient.

For those embodiments described herein which involve a multi-step method, the sample used in each step of the method may be the same sample obtained from the patient. When the method comprises multiple quantification steps, all the steps may be performed at the same time using the same sample.

The sample may be obtained from the patient before, during, and/or after treatment for the systemic inflammatory condition. In one embodiment, the sample is taken before treatment for the systemic inflammatory condition has been initiated. In one embodiment, the sample is taken after treatment for the systemic inflammatory condition has been initiated (eg. so as to monitor the effectiveness of a treatment regimen).

In one embodiment, the sample may be obtained from the patient at least 1 hour (eg. at least 2 hours, at least 4 hours, at least 6 hours, at least 8 hours, at least 12 hours, at least 24 hours, at least 36 hours, at least 48 hours, at least 72 hours, at least 96 hours, or at least 120 hours) after the patient presents with one or more clinical symptoms of a systemic inflammatory condition.

In one embodiment, the sample may be obtained from the patient up to 1 hour (eg. up to 2 hours, up to 4 hours, up to 6 hours, up to 8 hours, up to 12 hours, up to 24 hours, up to 36 hours, up to 48 hours, up to 72 hours, up to 96 hours, or up to 120 hours) after the patient presents with one or more clinical symptoms of a systemic inflammatory condition. For example, the sample may be obtained from the patient up to 24 hours after the patient presents with one or more clinical symptoms of a systemic inflammatory condition. For example, the sample may be obtained from the patient up to 48 hours after the patient presents with one or more clinical symptoms of a systemic inflammatory condition. For example, the sample may be obtained from the patient up to 72 hours after the patient presents with one or more clinical symptoms of a systemic inflammatory condition. For example, the sample may be obtained from the patient up to 96 hours after the patient presents with one or more clinical symptoms of a systemic inflammatory condition. For example, the sample may be obtained from the patient up to 120 hours after the patient presents with one or more clinical symptoms of a systemic inflammatory condition.

In one embodiment, the sample may be obtained from the patient between about 1 hour and 120 hours (eg. between about 1 hour and 96 hours, between about 1 hour and 72 hours, between about 1 hour and 48 hours, or between about 1 hour and 24 hours) after the patient presents with one or more clinical symptoms of a systemic inflammatory condition.

For example, the sample may be obtained from the patient between about 12 hours and 120 hours (eg. between about 12 hours and 96 hours, between about 12 hours and 72 hours, between about 12 hours and 48 hours, or between about 12 hours and 24 hours) after the patient presents with one or more clinical symptoms of a systemic inflammatory condition.

For example, the sample may be obtained from the patient between about 24 hours and 120 hours (eg. between about 24 hours and 96 hours, between about 24 hours and 72 hours, or between about 24 hours and 48 hours) after the patient presents with one or more clinical symptoms of a systemic inflammatory condition. For example, the sample may be obtained between about 24 hours and 48 hours. For example, the sample may be obtained between about 24 hours and 72 hours. For example, the sample may be obtained between about 24 hours and 96 hours.

For example, the sample may be obtained from the patient between about 48 hours and 120 hours (eg. between about 48 hours and 96 hours, or between about 48 hours and 72 hours) after the patient presents with one or more clinical symptoms of a systemic inflammatory condition.

For example, the sample may be obtained from the patient between about 72 hours and 120 hours or between about 72 hours and 96 hours, after the patient presents with one or more clinical symptoms of a systemic inflammatory condition.

For example, the sample may be obtained from the patient between about 96 hours and 120 hours after the patient presents with one or more clinical symptoms of a systemic inflammatory condition.

Presentation of the patient with one or more clinical symptoms of a systemic inflammatory condition means that the patient displays or presents with one or more (eg. 2 or more, 3 or more, or all 4) clinical symptoms of a systemic inflammatory condition. The skilled person will be aware of the clinical symptoms associated with a systemic inflammatory condition. Clinical symptoms of a systemic inflammatory condition include: (i) fever (temperature of 38.0° C. (100.4° F.) or more) or hypothermia (temperature of 36.0° C. (96.8° F.) or less); (ii) tachycardia (at least 90 beats per minute); (iii) tachypnea (at least 20 breaths per minute or PaCC >2 less than 4.3 kPa (32.0 mm Hg) or the need for mechanical ventilation); and (iv) an altered white blood cell (WBC) count of 12×10⁶ cells/mL or more, or an altered WBC count of 4×10⁶ cells/mL or less, or the presence of more than 10% band forms.

The patient does not necessarily have to present with one or more clinical symptoms of a systemic inflammatory condition before they are tested for the presence (or absence) of a systemic inflammatory condition.

Thus, in one embodiment, the sample may be obtained from the patient at least 1 hour (eg. at least 2 hours, at least 4 hours, at least 6 hours, at least 8 hours, at least 12 hours, at least 24 hours, at least 36 hours, at least 48 hours, at least 72 hours, at least 96 hours, or at least 120 hours) after the patient is admitted to a medical care facility.

In one embodiment, the sample may be obtained from the patient up to 1 hour (e.g., up to 2 hours, up to 4 hours, up to 6 hours, up to 8 hours, up to 12 hours, up to 24 hours, up to 36 hours, up to 48 hours, up to 72 hours, up to 96 hours, or up to 120 hours) after the patient is admitted to a medical care facility. For example, the sample may be obtained from the patient up to 24 hours after the patient is admitted to a medical care facility. For example, the sample may be obtained from the patient up to 48 hours after the patient is admitted to a medical care facility. For example, the sample may be obtained from the patient up to 72 hours after the patient is admitted to a medical care facility. For example, the sample may be obtained from the patient up to 96 hours after the patient is admitted to a medical care facility. For example, the sample may be obtained from the patient up to 120 hours after the patient is admitted to a medical care facility.

In one embodiment, the sample may be obtained from the patient between about 1 hour and 120 hours (eg, between about 1 hour and 96 hours, between about 1 hour and 72 hours, between about 1 hour and 48 hours, or between about 1 hour and 24 hours) after the patient is admitted to a medical care facility.

For example, the sample may be obtained from the patient between about 12 hours and 120 hours (eg, between about 12 hours and 96 hours, between about 12 hours and 72 hours, between about 12 hours and 48 hours, or between about 12 hours and 24 hours) after the patient is admitted to a medical care facility.

For example, the sample may be obtained from the patient between about 24 hours and 120 hours (eg, between about 24 hours and 96 hours, between about 24 hours and 72 hours, or between about 24 hours and 48 hours) after the patient is admitted to a medical care facility. For example, the sample may be obtained between about 24 hours and 48 hours. For example, the sample may be obtained between about 24 hours and 72 hours. For example, the sample may be obtained between about 24 hours and 96 hours.

For example, the sample may be obtained from the patient between about 48 hours and 120 hours (eg. between about 48 hours and 96 hours, or between about 48 hours and 72 hours) after the patient is admitted to a medical care facility.

For example, the sample may be obtained from the patient between about 72 hours and 120 hours or between about 72 hours and 96 hours, after the patient is admitted to a medical care facility.

For example, the sample may be obtained from the patient between about 96 hours and 120 hours after the patient is admitted to a medical care facility.

As used herein, the phrase “after the patient is admitted to a medical care facility” refers to the admission of a patient for clinical observation and/or treatment. Admission to a medical care facility includes admittance of the patient into hospital (eg. into an intensive care unit). The term “medical care facility” is not limited to hospitals, but includes any environment in which a patient can be clinically monitored and/or treated (eg. including doctors surgeries, or expedition medical tents).

As used herein, the term “biomarker” refers to virtually any biological compound, such as a protein and a fragment thereof, a peptide, a polypeptide, a proteoglycan, a glycoprotein, a lipoprotein, a carbohydrate, a lipid, a nucleic acid, an organic or inorganic chemical, a natural polymer, and a small molecule, that is present in the biological sample and that may be isolated from, or measured in, the biological sample. Furthermore, a biomarker can be the entire intact molecule, or it can be a portion thereof that may be partially functional or recognized, for example, by an antibody or other specific binding protein.

In one embodiment, the one or more biomarker is a nucleic acid (e.g., DNA, such as cDNA or amplified DNA, or RNA, such as mRNA). The one or more biomarker may have a nucleic acid sequence as shown in the sequences in the Sequence Information section herein. The relevant sequence identifiers are also shown in Tables 1-4.

In another embodiment, the one or more biomarker is a protein. As used herein, the terms “protein”, “peptide”, and “polypeptide” are, unless otherwise indicated, interchangeable. When the presence and/or amount of two or more biomarkers are determined, the biomarkers may all be protein biomarkers or all nucleic acid biomarkers. Alternatively, the biomarkers may be both protein and nucleic acid biomarkers.

The present invention also encompasses, without limitation, polymorphisms, isoforms, metabolites, mutants, variants, derivatives, modifications, subunits, fragments, protein-ligand complexes and degradation products of the biomarkers listed in Tables 1-4.

The protein fragments can be 200, 150, 100, 50, 25, 10 amino acids or fewer in length. The nucleic acid fragments can be 1000, 500, 250 150, 100, 50, 25, 10 nucleotides or fewer in length.

Variants of the protein biomarkers of the present invention include polypeptides with altered amino acid sequences due to amino acid substitutions, deletions, or insertions. Variant polypeptides may comprise conservative or non-conservative amino acid substitutions, deletions or additions. Variants include polypeptides that have an amino acid sequence being at least 70%, at least 80%, at least 90%, at least 95%, at least 98% or at least 99% identical to the amino acid sequences of the polypeptides listed in Tables 1-4. Variants may be allelic variants, splice variants or any other species specific homologs, paralogs, or orthologs.

Derivatives of the protein biomarkers of the present invention are polypeptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

Variants of the nucleic acid biomarkers of the present invention may have a sequence identity of at least 80% with the corresponding nucleic acid sequence shown in the Sequence Information section. Sequence identity may be calculated as described herein. A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

The one or more inflammation biomarker used in the method may be selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1.

In one embodiment, the one or more inflammation biomarker may be selected from the group consisting of: IGFBP2, CYP19A1, and VSTM1. In one embodiment, the one or more biomarker may be selected from the group consisting of: CD177, IL10, IL1R1, IL1R2, VSTM1, ADM, and HP, wherein said biomarkers are associated with immune response and/or inflammation. In one embodiment, the one or more biomarker may be selected from the group consisting of: METTL7B, RETN, and CYP19A1, wherein said biomarkers are associated with lipid metabolism. In one embodiment, the one or more biomarker may be selected from the group consisting of: MMP9 and MMP8, wherein said biomarkers are associated with extracellular matrix maintenance or composition.

Each of the biomarkers for a systemic inflammatory condition may be used alone, or in combination with any of the biomarkers for a systemic inflammatory condition in the method of the invention. For example, any combination of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more 19 or more, 20 or more, up to and including all of the biomarkers may be used to diagnose a systemic inflammatory condition according to the method of the invention.

In one embodiment, the one or more biomarker is FAM20A. In one embodiment, the one or more biomarker is OLAH. In one embodiment, the one or more biomarker is CD177. In one embodiment, the one or more biomarker is ADM. In one embodiment, the one or more biomarker is IL10. In one embodiment, the one or more biomarker is METTL7B. In one embodiment, the one or more biomarker is MMP9. In one embodiment, the one or more biomarker is RETN. In one embodiment, the one or more biomarker is TDRD9. In one embodiment, the one or more biomarker is ITGA7. In one embodiment, the one or more biomarker is BMX. In one embodiment, the one or more biomarker is HP. In one embodiment, the one or more biomarker is IGFBP2. In one embodiment, the one or more biomarker is ALPL. In one embodiment, the one or more biomarker is DACH1. In one embodiment, the one or more biomarker is IL1R1. In one embodiment, the one or more biomarker is IL1R2. In one embodiment, the one or more biomarker is CYP19A1. In one embodiment, the one or more biomarker is MMP8. In one embodiment, the one or more biomarker is TGFA. In one embodiment, the one or more biomarker is VSTM1.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more 19 or more, 20 of more, or all 21) of the biomarkers selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1, may be used to diagnose a systemic inflammatory condition in a patient.

As demonstrated by ROC analysis performed in Example 2, the inflammation biomarkers FAM20A, OLAH and CD177 were all shown to provide highly accurate diagnosis of patients having a systemic inflammatory condition when used on their own or in combination. In one embodiment, a combination of FAM20A and OLAH may be used to diagnose a systemic inflammatory condition in a patient. In one embodiment, a combination of FAM20A, OLAH and CD177 may be used to diagnose a systemic inflammatory condition in a patient.

One or more additional biomarker for inflammation may also be used in the method of the invention to diagnose a systemic inflammatory condition. Any combination of the one or more additional biomarker may be used in combination with the one or more biomarker of the invention. For example at least 1, 2, 3, 4, 5, 6, 7, 8, 9, 10, 11, 12, 13, 14, 15, 16, 17, 18, 19, or all 20 additional biomarkers for inflammation may be used in combination with the one or more biomarker of the invention (as described herein). Typically, the one or more additional biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1.

In one embodiment, the one or more biomarker is FAM20A, and the one or more additional biomarker is selected from at least 1 (eg. at least 2, 3, 4, 5, 6, 7, 8, 9, 10, 11, 12, 13, 14, 15, 16, 17, 18, 19, up to and including all) of the biomarkers: OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1.

In one embodiment, the one or more biomarker is OLAH, and the one or more additional biomarker is selected from at least 1 (eg. at least 2, 3, 4, 5, 6, 7, 8, 9, 10, 11, 12, 13, 14, 15, 16, 17, 18, 19, up to and including all) of the biomarkers: FAM20A, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1.

In one embodiment, the one or more biomarker is CD177, and the one or more additional biomarker is selected from at least 1 (eg. at least 2, 3, 4, 5, 6, 7, 8, 9, 10, 11, 12, 13, 14, 15, 16, 17, 18, 19, up to and including all) of the biomarkers: FAM20A, OLAH, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1.

A biomarker is considered to be informative if a measurable aspect or characteristic of the biomarker is associated with a given state of an individual, such as the diagnosis, monitoring or prognosis of a systemic inflammatory condition. Such a measurable aspect or characteristic may include, for example, the presence, absence, or concentration of the biomarker in the biological sample from the individual and/or its presence as part of a profile of biomarkers. Such a measurable aspect of a biomarker is defined herein as a “feature.” For example, the presence of a biomarker in a sample may be a feature. As another example, the amount of a biomarker in a sample, or the amount of a biomarker in a sample compared with a control or reference sample may be a feature. A feature may also be a ratio of two or more measurable aspects of biomarkers, which biomarkers may or may not be of known identity. A “biomarker profile” comprises at least two such features, where the features can correspond to the same or different classes of biomarkers such as, for example, two nucleic acids or a nucleic acid and a protein. A biomarker profile may also comprise at least three, four, five, 10, 20, 30 or more features. In one embodiment, a biomarker profile comprises hundreds, or even thousands, of features. In another embodiment, the biomarker profile comprises at least one measurable aspect of at least one internal standard.

A “phenotypic change” is a detectable change in a parameter associated with a given state of the individual. For instance, a phenotypic change may include an increase or decrease of a biomarker in a bodily fluid, where the change is associated with a systemic inflammatory condition (such as sepsis or SIRS) or distinguishing between sepsis and SIRS. The presence and/or amount of each of the one or more biomarkers of the invention is a feature or phenotypic change according to the present invention. For example, the presence of each of the one or more biomarkers of the invention is a feature or phenotypic change according to the present invention. For example, the amount of each of the one or more biomarkers of the invention is a feature or phenotypic change according to the present invention. In a further example, the presence and amount of each of the one or more biomarkers of the invention is a feature or phenotypic change according to the present invention.

A phenotypic change may further include a change in a detectable aspect of a given state of the individual that is not a change in a measurable aspect of a biomarker. For example, a change in phenotype may include a detectable change in body temperature, weight loss, fatigue, respiration rate or other physiological parameter. Such changes can be determined via clinical observation and measurement using conventional techniques that are well-known to the skilled artisan. As used herein, “conventional techniques” are those techniques that classify an individual based on phenotypic changes without obtaining a biomarker profile according to the present invention.

According to the present invention, systemic inflammatory conditions may be diagnosed, monitored, and/or prognosed by obtaining a profile of biomarkers from a sample obtained from a patient. As used herein, “obtain” means “to come into possession of”.

A feature as defined herein for the diagnosis, monitoring or prognosis of a systemic inflammatory condition may be detected, quantified or determined by any appropriate means. For example, the one or more biomarker of the invention, a measurable aspect or characteristic of the one or more biomarker or a biomarker profile of the invention may be detected by any appropriate means. The presence of the one or more biomarkers of the invention may be considered together as a “biomarker profile” of the invention. The presence of the individual biomarkers within any of the biomarker combinations disclosed herein may be considered together as a “biomarker profile” of the invention.

The presence and/or amount of the one or more biomarker of the invention may be determined by quantitative and/or qualitative analysis. Measurement of the one or more biomarkers can be performed by any method that provides satisfactory analytical specificity, sensitivity and precision. The invention encompasses the use of those methods known to a person skilled in the art to measure the presence and/or amount of one or more biomarkers.

In one embodiment, the methods described herein involve determining the “presence and amount of the one or more biomarker”. In one embodiment, the methods described herein involve determining the “presence of the one or more biomarker”. In one embodiment, the methods described herein involve determining the “amount of the one or more biomarker”.

Determining the “amount of one or more biomarker” in a sample means quantifying the biomarker by determining the relative or absolute amount of the biomarker. The amount of the one or more biomarker of the invention encompasses the mass of the one or more biomarker, the molar amount of the one or more biomarker, the concentration of the one or biomarker and the molarity of the one or more biomarker. This amount may be given in any appropriate units. For example, the concentration of the one or more biomarker may be given in pg/ml, ng/ml or μg/ml. It will be appreciated that the assay methods do not necessarily require measurement of absolute values of biomarker, unless it is desired, because relative values are sufficient for many applications of the invention. Accordingly, the “amount” can be the “absolute” total amount of the biomarker that is detected in a sample, or it can be a “relative” amount, e.g., the difference between the biomarker detected in a sample and e.g., another constituent of the sample. In some embodiments, the amount of the biomarker may be expressed by its concentration in a sample, or by the concentration of an antibody that binds to the biomarker. Thus, the actual amount of the one or more biomarker, such as the mass, molar amount, concentration or molarity of the one or biomarker may be assessed and compared with the corresponding reference value. Alternatively, the amount of one or more biomarker may be compared with that of the reference value without quantifying the mass, molar amount, concentration or molarity of the one or more biomarker.

The presence and/or amount of the one or more biomarker can be determined at the protein or nucleic acid level using any method known in the art. The particular preferred method for determining the presence and/or amount of the one or more biomarkers will depend in part on the identity and nature of the biomarker.

The biomarkers of the invention may be detected at the nucleic acid or protein level. Thus, the biomarkers of the invention may be DNA, RNA or protein and may be detected using any appropriate technique. The presence and/or amount of the one or more biomarker of the invention may be measured directly or indirectly. Any appropriate agent may be used to determine the presence and/or amount of the one or more biomarker of the invention. For example, the presence and/or amount of the one or more biomarker of the invention may be determined using an agent selected from peptides and peptidomimetics, antibodies, small molecules and single-stranded DNA or RNA molecules, as described herein. Suitable standard techniques are known in the art.

For example, when the one or more biomarker is detected at the nucleic acid level this may be carried out using: (i) biomarker-specific oligonucleotide DNA or RNA or any other nucleic acid derivative probes bound to a solid surface; (ii) purified RNA (labelled by any method, for example using reverse transcription and amplification) hybridised to probes; (iii) whole lysed blood, from which the RNA is labelled by any method and hybridised to probes; (iv) purified RNA hybridised to probes and a second probe (labelled by any method) hybridised to the purified RNA; (v) whole lysed blood from which the RNA is hybridised to probes, and a second probe (labelled by any method) which is hybridised to the RNA; (vi) purified peripheral blood leukocytes, obtaining purified RNA (labelled by any method), and hybridising the purified labelled RNA to probes; (vii) purified peripheral blood leukocytes, obtaining purified RNA and hybridising the RNA to probes, then using a second probe (labelled by any method) which hybridises to the RNA; (viii) RT-PCR using any primer/probe combination or inter-chelating fluorescent label, for example SYBRGreen; (ix) end-point PCR; (x) digital PCR; (xi) sequencing; (xii) array cards (RT-PCR); (xiii) lateral flow devices/methodology; and/or (xiv) digital microfluidics.

In one embodiment, quantitative real-time PCR is used to determine the presence and/or amount of the one or more biomarker of the invention. Quantitative real-time PCR may be performed using forward and reverse oligonucleotide primers that amplify the target sequence (such as those described herein). Detection of the amplified product is done in real-time, and may be performed using oligonucleotide probes that produce a fluorescent signal when the target DNA is amplified (e.g., Taqman® fluorogenic probes), or using SYBR Green dye that binds to double-stranded DNA and emits fluorescence when bound.

In one embodiment, oligonucleotide microarray analysis is used to detect and/or quantify the one or more biomarker of the invention using biomarker-specific oligonucleotide DNA or RNA or any other nucleic acid derivative probes bound to a solid surface.

In a preferred embodiment, RNA from a sample (either purified or unpurified) is labelled via any method (typically amplification) and used to interrogate one or more probe immobilised on a surface. Typically, the one or more probes are 50 to 100 nucleotides in length.

In another preferred embodiment, one or more probe is immobilised on a surface and the RNA from a sample is hybridised to one or more second probe (labelled by any method). The RNA hybridised with the second (labelled) probe is then used to interrogate the one or more probe immobilised on the surface. Examples of such methodology are known in the art, including the Vantix™ system.

For example, when the one or more biomarker is detected at the protein acid level this may be carried out using: (i) biomarker-specific primary antibodies or antibody fragments bound to a solid surface; (ii) whole lysed blood biomarker antigen bound to antibodies or antibody fragments; (iii) secondary biomarker-specific antibodies or antibody fragments used to detect biomarker antigen bound to primary antibody (labelled using any method); (iv) biomarker-specific primary aptamers bound to a solid surface; (v) whole lysed blood—biomarker antigen bound to aptamers; (vi) secondary biomarker-specific aptamer used to detect biomarker antigen bound to primary aptamer (labelled using any method); (vii) any antibody derivative i.e. phage display etc. used as above; (viii) lateral flow devices/methodology; (ix) chromatography; (x) mass spectrometry; (xi) nuclear magnetic resonance (NMR); (xii) protein gels/transfers to filter; and/or (xiii) immunoprecipitation. In a preferred embodiment, a lateral flow device may be used to detect the one or more protein biomarker.

Any agent for the detection of or for the determination of the amount of the one or more biomarker of the invention may be used to determine the amount of the one or more biomarker. Similarly, any method that allows for the detecting of the one or more biomarker, the quantification, or relative quantification of the one or more biomarker may be used.

Agents for the detection of or for the determination of the amount of one or more biomarker may be used to determine the amount of the one or more biomarker in a sample obtained from the patient. Such agents typically bind to the one or more biomarker. Such agents may bind specifically to the one or more biomarker. The agent for the detection of or for the determination of the amount of the one or more biomarker may be an antibody or other binding agent specific for the one or more biomarker. By specific, it will be understood that the agent or antibody binds to the molecule of interest, in this case the one or more biomarker, with no significant cross-reactivity to any other molecule, particularly any other protein. Cross-reactivity may be assessed by any suitable method. Cross-reactivity of an agent or antibody for the one or more biomarker with a molecule other than the one or more biomarker may be considered significant if the agent or antibody binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the one or more biomarker. Preferably, the agent or antibody binds to the other molecule at less than 20%, less than 15%, less than 10% or less than 5%, less than 2% or less than 1% the strength that it binds to the one or more biomarker.

As described herein, the presence and/or amount of the one or more biomarker, and hence the biomarker profile may be determined immunologically by reacting antibodies, or functional fragments thereof, specific to the biomarkers. A functional fragment of an antibody is a portion of an antibody that retains at least some ability to bind to the antigen to which the complete antibody binds. The fragments, which include, but are not limited to, scFv fragments, Fab fragments, F(ab) fragments and F(ab)2 fragments, can be recombinantly produced or enzymatically produced. Specific binding molecules other than antibodies, such as aptamers, may be used to bind the biomarkers.

The antibody may be monoclonal or polyclonal. The antibody may be produced by any suitable method known in the art. For example, polyclonal antibodies may be obtained by immunising a mammal, typically a rabbit or a mouse, with the one or more biomarker under suitable conditions and isolating antibody molecules from, for example, the serum of said mammal. Monoclonal antibodies may be obtained by hybridoma or recombinant methods.

Hybridoma methods may involve immunising a mammal, typically a rabbit or a mouse, with the one or more biomarker under suitable conditions, then harvesting the spleen cells of said mammal and fusing them with myeloma cells. The mixture of fused cells is then diluted, and clones are grown from single parent cells. The antibodies secreted by the different clones are then tested for their ability to bind to the one or more biomarker, and the most productive and stable clone is then grown in culture medium to a high volume. The secreted antibody is collected and purified.

Recombinant methods may involve the cloning into phage or yeast of different immunoglobulin gene segments to create libraries of antibodies with slightly different amino acid sequences. Those sequences which give rise to antibodies which bind to the one or more biomarker may be selected and the sequences cloned into, for example, a bacterial cell line, for production.

Typically, the antibody is a mammalian antibody, such as a primate, human, rodent (e.g. mouse or rat), rabbit, ovine, porcine, equine or camel antibody. The antibody may be a camelid antibody or shark antibody. The antibody may be a nanobody. The antibody can be any class or isotype of antibody, for example IgM, but is preferably IgG. The antibody may be a humanised antibody.

The antibody or fragment may be associated with other moieties, such as linkers which may be used to join together 2 or more fragments or antibodies. Such linkers may be chemical linkers or can be present in the form of a fusion protein with the fragment or whole antibody. The linkers may thus be used to join together whole antibodies or fragments which have the same or different binding specificities, e.g., that can bind the same or different polymorphisms. The antibody may be a bispecific antibody which is able to bind to two different antigens, typically any two of the polymorphisms mentioned herein. The antibody may be a ‘diabody’ formed by joining two variable domains back to back. In the case where the antibodies used in the method are present in any of the above forms which have different antigen binding sites of different specificities then these different specificities are typically to polymorphisms at different positions or on different proteins. In one embodiment the antibody is a chimeric antibody comprising sequence from different natural antibodies, for example a humanised antibody.

Methods to assess an amount of the one or more biomarker may involve contacting a sample with an agent or antibody capable of binding specifically to the one or more biomarker. Such methods may include dipstick assays and Enzyme-linked Immunosorbant Assay (ELISA), or similar assays, such as those using a lateral flow device. Other immunoassay types may also be used to assess the one or more biomarker amounts. Typically, dipsticks comprise one or more antibodies or proteins that specifically bind to the one or more biomarker. If more than one antibody is present, the antibodies preferably have different non-overlapping determinants such that they may bind to the one or more biomarker simultaneously.

ELISA is a heterogeneous, solid phase assay that requires the separation of reagents. ELISA is typically carried out using the sandwich technique or the competitive technique. The sandwich technique requires two antibodies. The first specifically binds the one or more biomarker and is bound to a solid support. The second antibody is bound to a marker, typically an enzyme conjugate. A substrate for the enzyme is used to quantify the one or more biomarker-antibody complex and hence the amount of the one or more biomarker in a sample. The antigen competitive inhibition assay also typically requires a one or more biomarker-specific antibody bound to a support. A biomarker-enzyme conjugate is added to the sample (containing the one or more biomarker) to be assayed. Competitive inhibition between the biomarker-enzyme conjugate and unlabelled biomarker allows quantification of the amount of the one or more biomarker in a sample. The solid supports for ELISA reactions preferably contain wells.

Antibodies capable of binding specifically to the one or more biomarker may be used in methods of immunofluorescence to detect the presence of the one or more biomarker.

The present invention may also employ methods of determining the amount of the one or more biomarker that do not comprise antibodies. High Performance Liquid Chromatography (HPLC) separation and fluorescence detection is preferably used as a method of determining the amount of the one or more biomarker. HPLC apparatus and methods as described previously may be used (Tsikas D et al., J Chromatogr B Biomed Sci Appl 1998; 705: 174-6). Separation during HPLC is typically carried out on the basis of size or charge. Prior to HPLC, endogenous amino acids and an internal standard L-homoarginine are typically added to assay samples and these are phase extracted on CBA cartridges (Varian, Harbor City, Calif.). Amino acids within the samples are preferably derivatized with o-phthalaldehyde (OPA). The accuracy and precision of the assay is preferably determined within quality control samples for all amino acids.

Other methods of determining the amount the one or more biomarker that do not comprise antibodies include mass spectrometry. Mass spectrometric methods may include, for example, matrix-assisted laser desorption/ionization mass spectrometry (MALDI MS), surface-enhanced laser desorption/ionization mass spectrometry (SELDI MS), time of flight mass spectrometry (TOF MS) and liquid chromatography mass spectrometry (LC MS).

A separation method may be used to determine the presence and/or amount of the one or more biomarker and hence to create a profile of biomarkers, such that only a subset of biomarkers within the sample is analysed. For example, the biomarkers that are analysed in a sample may consist of mRNA species from a cellular extract, which has been fractionated to obtain only the nucleic acid biomarkers within the sample, or the biomarkers may consist of a fraction of the total complement of proteins within the sample, which have been fractionated by chromatographic techniques. One or more, two or more, three or more, four or more, or five or more separation methods may be used according to the present invention.

Determination of the presence and/or amount of the one or more biomarker, and hence the creation of a profile of biomarkers may be carried out without employing a separation method. For example, a biological sample may be interrogated with a labelled compound that forms a specific complex with a biomarker in the sample, where the intensity of the label in the specific complex is a measurable characteristic of the biomarker. A suitable compound for forming such a specific complex is a labelled antibody. A biomarker may be measured using an antibody with an amplifiable nucleic acid as a label. The nucleic acid label may become amplifiable when two antibodies, each conjugated to one strand of a nucleic acid label, interact with the biomarker, such that the two nucleic acid strands form an amplifiable nucleic acid.

The presence and/or amount of the one or more biomarker, and hence the biomarker profile may be derived from an assay, such as an array, of nucleic acids, where the biomarkers are the nucleic acids or complements thereof. For example, the biomarkers may be ribonucleic acids. The presence and/or amount of the one or more biomarker, and hence the biomarker profile may be obtained using a method selected from nuclear magnetic resonance, nucleic acid arrays, dot blotting, slot blotting, reverse transcription amplification and Northern analysis.

The determination of the presence and/or amount of the one or more biomarker, and hence a biomarker profile may be generated by the use of one or more separation methods. For example, suitable separation methods may include a mass spectrometry method, such as electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SLMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n. Other mass spectrometry methods may include, inter alia, quadrupole, fourier transform mass spectrometry (FTMS) and ion trap. Other suitable separation methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. The sample may be fractionated prior to application of the separation method.

The determination of the presence and/or amount of the one or more biomarker, and hence a biomarker profile may be generated by methods that do not require physical separation of the biomarkers themselves. For example, nuclear magnetic resonance (NMR) spectroscopy may be used to resolve a profile of biomarkers from a complex mixture of molecules. An analogous use of NMR to classify tumours is disclosed in Hagberg, NMR Biomed. 11: 148-56 (1998), for example. Additional procedures include nucleic acid amplification technologies, which may be used to generate a profile of biomarkers without physical separation of individual biomarkers. (See Stordeur et al., J. Immunol. Methods 259:55-64 (2002) and Tan et al., Proc. Nat'l Acad. Sci. USA 99: 11387-11392 (2002), for example.)

In one embodiment, laser desorption/ionization time-of-flight mass spectrometry is used to determine the presence and/or amount of the one or more biomarker, and hence create a biomarker profile where the biomarkers are proteins or protein fragments that have been ionized and vaporized off an immobilizing support by incident laser radiation. A profile is then created by the characteristic time-of-flight for each protein, which depends on its mass-to-charge (“m/z”) ratio. A variety of laser desorption/ionization techniques are known in the art. (See, e.g., Guttman et al., Anal Chem. 73: 1252-62 (2001) and Wei et al., Nature 399: 243-46 (1999).)

Laser desorption/ionization time-of-flight mass spectrometry allows the generation of large amounts of information in a relatively short period of time. A sample is applied to one of several varieties of a support that binds all of the biomarkers, or a subset thereof, in the sample. Cell lysates or samples are directly applied to these surfaces in volumes as small as 0.5 μL, with or without prior purification or fractionation. The lysates or sample can be concentrated or diluted prior to application onto the support surface. Laser desorption/ionization is then used to generate mass spectra of the sample, or samples, in as little as three hours.

In a preferred embodiment, the total mRNA from a cellular extract of the patient is assayed, and the various mRNA species that are obtained from the sample are used as biomarkers. Biomarker profiles may be obtained, for example, by hybridizing these mRNAs to an array of probes, which may comprise oligonucleotides or cDNAs, using standard methods known in the art. Alternatively, the mRNAs may be subjected to gel electrophoresis or blotting methods such as dot blots, slot blots or Northern analysis, all of which are known in the art. (See, e.g., Sambrook et al. in “Molecular Cloning, 3rd ed.,” Cold Spring Harbor Laboratory Press, Cold Spring Harbor, N.Y. (2001).) mRNA profiles also may be obtained by reverse transcription followed by amplification and detection of the resulting cDNAs, as disclosed by Stordeur et al, supra, for example. In another embodiment, the profile may be obtained by using a combination of methods, such as a nucleic acid array combined with mass spectroscopy.

Different methods have different advantages and may be preferred depending on numerous factors, such as the particular circumstances of the patients to be tested and/or the availability of reagents/equipment in the diagnostics laboratory. For example, qPCR using probe/quencher hydrolysis probes is highly specific and stringent. As another example, microarray analysis can resolve subtle differences in expression of transcript variants, which may be important in disease pathology and diagnosis.

Different one or more biomarkers may be used with different detection methods according to the present invention.

When the amount of two or more biomarkers are determined, the amount of each biomarker may be determined, or the cumulative amount of all the biomarkers may be determined. Alternatively, the amount of the two or more biomarkers can be combined with each other in a formula to form an index value.

In the methods and uses of the invention, the presence and/or amount of the one or more biomarker of the invention in a patient (or the profile of biomarkers in a patient) may be measured relative to a corresponding reference value. As such, the presence and/or amount of the one of more biomarker of the invention (or the profile of biomarkers) may be “compared” to a corresponding reference value.

The terms “comparison”, “comparing” and “compared” are used herein interchangeably, and includes any means to discern at least one difference in the presence and/or amount of the one or more biomarker in the test sample as compared to a reference value (or as compared to a further sample obtained from the patient where monitoring of a systemic inflammatory condition takes place). In one embodiment, the methods of the invention described herein may involve comparison of “the amount of the one or more biomarker” in the test sample as compared to a reference value. In one embodiment, the methods of the invention described herein may involve comparison of “the presence and amount of the one or more biomarker” in the test sample as compared to a reference value.

A comparison may include a visual inspection of chromatographic spectra, and a comparison may include arithmetical or statistical comparisons of values assigned to the features of the profiles. Such statistical comparisons include, but are not limited to, applying a decision rule. If the biomarker profiles comprise at least one internal standard, the comparison to discern a difference in the biomarker profiles may also include features of these internal standards, such that features of the biomarker are correlated to features of the internal standards. As described herein, the comparison can be used to diagnose, monitor or prognose a systemic inflammatory condition, such as sepsis, abdominal sepsis, pulmonary sepsis or SIRS, and can be used to distinguish between sepsis and SIRS in a patient, or it can be used to distinguish between abdominal sepsis and pulmonary sepsis in a patient.

The term “reference value” refers to a value that is representative of a control individual or population whose disease state is known. A reference value can be determined for any particular population, subpopulation, or group of individuals according to standard methods well known to those of skill in the art. The actual amount of the one or more biomarkers, such as the mass, molar amount, concentration or molarity of the one or more biomarker of the invention may be assessed and compared with the corresponding reference population. Alternatively, the amount of one or more biomarker of the invention may be compared with that of the reference population without quantifying the mass, molar amount, concentration or molarity of the one or more biomarker.

The reference value may be obtained from a healthy individual or a population of healthy individuals, eg. by quantifying the amount of the one or more biomarker in a sample obtained from the healthy individual or the population of healthy individuals. As used herein, “healthy” refers to a subject or group of individuals who are in a healthy state, e.g., patients who have not shown any symptoms of the disease, have not been previously diagnosed with the disease and/or are not likely to develop the disease. In one embodiment, the healthy individual (or population of healthy individuals) is not on medication affecting the disease and has not been diagnosed with any other disease. In one embodiment, the healthy individual (or population of healthy individuals) has similar sex, age and body mass index (BMI) as compared with the test patient. In one embodiment, the healthy individual (or population of healthy individuals) does not have a current infection or a chronic infection. Application of standard statistical methods used in medicine permits determination of normal levels of expression, as well as significant deviations from such normal levels.

The reference value may be obtained from an individual or a population of individuals suffering from the disease, eg. by quantifying the amount of the one or more biomarker in a sample obtained from the individual or the population of individuals suffering from the disease. The reference data is typically collected from individuals that present at a medical centre with clinical signs relating to the relevant disease of interest. The reference value may be obtained, for example, from an individual or population of individuals having a systemic inflammatory condition, such as those having sepsis (including those having abdominal sepsis or pulmonary sepsis) or those having SIRS. Such individual(s) may have similar sex, age and body mass index (BMI) as compared with the test patient.

In one embodiment, the reference value is obtained from an individual or population of individuals having sepsis. In one embodiment, the reference value is obtained from an individual or population of individuals having abdominal sepsis. In one embodiment, the reference value is obtained from an individual or population of individuals having pulmonary sepsis. In one embodiment, the individual (or population of individuals) presents at hospital with sepsis (such as abdominal sepsis or pulmonary sepsis) of less than 72 hours duration. The reference values may be obtained from individuals having sepsis may be obtained at any stage in the progression of sepsis, such as infection, bacteremia, severe sepsis, septic shock of multiple organ failure. For example, the reference values may be obtained from patients having severe sepsis and/or septic shock. Diagnosis of sepsis (such as severe sepsis and/or septic shock) is based on the conventional diagnosis methods defined herein.

In one embodiment, the reference value is obtained from an individual or a population of individuals having SIRS. Diagnosis of SIRS is based on the SIRS criteria defined herein. In one embodiment, the individual (or population of individuals) may have organ failure defined as SOFA score >2. In one embodiment, the individual or a population of individuals having SIRS has not been treated with antibiotics for treatment of known or suspected infection. In one embodiment, the individual or a population of individuals having SIRS have been admitted to a medical care facility following out-of hospital cardiac arrest.

The reference value may be obtained from an individual or a population of individuals who are diagnosed as having sepsis (eg. abdominal or pulmonary sepsis) or SIRS by conventional methods about 24, 48, 72, 96 or 120 hours or more after biological samples were taken for the purpose of generating a reference sample. In one embodiment, the individual or a population of individuals is diagnosed as having sepsis (eg. abdominal or pulmonary sepsis) or SIRS using conventional methods about 24-48 hours, about 48-72 hours, about 72-96 hours, or about 96-120 hours after the biological samples were taken. Conventional methods for confirming diagnosis of sepsis and SIRS are as defined herein.

The sample(s) used to generate the reference values may be obtained from the individual (or population of individuals) that present at a medical centre with clinical signs relating to the relevant disease of interest at any of the time points described herein for sample collection from the test patient. All embodiments described herein for the timing of sample collection from a test patient thus apply equally to the time point at which samples are obtained from the reference individual (or population of individuals) for the purpose of generating a reference value. For example, the sample used to generate the reference value may be obtained from an individual (or population of individuals) up to 24 hours after the individual (or population of individuals) presents with one or more clinical symptoms of a systemic inflammatory condition, or is admitted to a medical care facility. The individual (or population of individuals) from which the sample is obtained is then later on confirmed as having a systemic inflammatory condition using the conventional methods described herein.

In one embodiment, the reference values used in the comparison step of the method are generated from a sample obtained at the same time point (or time period) as the sample obtained from the test patient. For example, if a sample is obtained from a test patient up to 24 hours after the patient presents with one or more clinical symptoms of a systemic inflammatory condition or is admitted to a medical care facility, then the corresponding reference value may be obtained from an individual (or population of individuals) up to 24 hours after the individual (or population of individuals) presents with one or more clinical symptoms of a systemic inflammatory condition or is admitted to a medical care facility. Likewise, if a sample is obtained from a test patient up to 48 hours after the patient presents with one or more clinical symptoms of a systemic inflammatory condition or is admitted to a medical care facility, then the corresponding reference value may be obtained from an individual (or population of individuals) up to 48 hours after the individual (or population of individuals) presents with one or more clinical symptoms of a systemic inflammatory condition or is admitted to a medical care facility.

The individuals from which samples are obtained for generation of reference data may be subject to further follow-on consultations to confirm clinical assessments, as well as to identify further changes in biomarkers, changes in the severity of clinical signs over a period of time, and/or survival outcome. The reference data collected may include series data to indicate the progression or regression of the disease, so that the data can be used to determine if the condition of a test individual is improving, worsening or static. The reference data collected from patients that recover from the systemic inflammatory disease, can be used as a reference value that is representative of an individual having a (good) prognosis of recovery from the systemic inflammatory condition. The reference data collected from patients that do not recover from the systemic inflammatory disease, can be used as a reference value that is representative of an individual having a prognosis of non-recovery from the systemic inflammatory condition (or a poor prognosis of recovery from the systemic inflammatory condition).

Multiple separate reference values may be used in the methods of the invention. For example, reference values may include those that are representative of one or more of (eg. two or more, three or more, four or more, or all five of): (i) an individual (or a population of individuals) having sepsis, (ii) an individual (or a population of individuals) having SIRS; (iii) an individual (or a population of individuals) having abdominal sepsis; (iv) an individual (or a population of individuals) having pulmonary sepsis; and/or (v) a healthy individual (or a population of healthy individuals).

For example, the methods of the invention may use reference values that are representative of: (i) an individual (or a population of individuals) having sepsis; (ii) an individual (or a population of individuals) having SIRS; and (iii) a healthy individual (or a population of healthy individuals). For example, the methods of the invention may use reference values that are representative of: (i) an individual (or a population of individuals) having sepsis; and (ii) an individual (or a population of individuals) having SIRS. For example, the methods of the invention may use reference values that are representative of: (i) an individual (or a population of individuals) having sepsis; and (ii) a healthy individual (or a population of healthy individuals). For example, the methods of the invention may use reference values that are representative of: (i) an individual (or a population of individuals) having SIRS; and (ii) a healthy individual (or a population of healthy individuals).

For example, the methods of the invention may use reference values that are representative of: (i) an individual (or a population of individuals) having abdominal sepsis; (ii) an individual (or a population of individuals) having pulmonary sepsis; (iii) an individual (or a population of individuals) having SIRS; and (iv) a healthy individual (or a population of healthy individuals). For example, the methods of the invention may use reference values that are representative of: (i) an individual (or a population of individuals) having abdominal sepsis; (ii) an individual (or a population of individuals) having pulmonary sepsis; and (iii) an individual (or a population of individuals) having SIRS. For example, the methods of the invention may use reference values that are representative of: (i) an individual (or a population of individuals) having abdominal sepsis; (ii) an individual (or a population of individuals) having pulmonary sepsis; and (iii) a healthy individual (or a population of healthy individuals). For example, the methods of the invention may use reference values that are representative of: (i) an individual (or a population of individuals) having abdominal sepsis; (ii) an individual (or a population of individuals) having SIRS; and (iii) a healthy individual (or a population of healthy individuals). For example, the methods of the invention may use reference values that are representative of: (i) an individual (or a population of individuals) having pulmonary sepsis; (ii) an individual (or a population of individuals) having SIRS; and (iii) a healthy individual (or a population of healthy individuals). For example, the methods of the invention may use reference values that are representative of: (i) an individual (or a population of individuals) having abdominal sepsis; and (ii) an individual (or a population of individuals) having pulmonary sepsis. For example, the methods of the invention may use reference values that are representative of: (i) an individual (or a population of individuals) having abdominal sepsis; and (ii) a healthy individual (or a population of healthy individuals). For example, the methods of the invention may use reference values that are representative of: (i) an individual (or a population of individuals) having pulmonary sepsis; and (ii) a healthy individual (or a population of healthy individuals). For example, the methods of the invention may use reference values that are representative of: (i) an individual (or a population of individuals) having abdominal sepsis; and (ii) an individual (or a population of individuals) having SIRS. For example, the methods of the invention may use reference values that are representative of: (i) an individual (or a population of individuals) having pulmonary sepsis; and (ii) an individual (or a population of individuals) having SIRS.

The reference value may be obtained from the same (test) patient, provided that the test and reference values are generated from biological samples taken at different time points and compared to one another. For example, a sample may be obtained from a patient at the start of a study period. A reference value taken from that sample may then be compared to biomarker profiles generated from subsequent samples from the same patient. Such a comparison may be used, for example, to monitor a systemic inflammatory condition (i.e., determine the progression of a systemic inflammatory condition in the patient by repeated classifications over time). Although the invention does not require a monitoring period to classify a patient, it will be understood that repeated classifications of the patient, i.e., repeated snapshots, may be taken over time until the individual is no longer at risk. Alternatively, a profile of biomarkers obtained from the patient may be compared to one or more profiles of biomarkers obtained from the same patient at different points in time.

In one embodiment, the reference value is obtained from a single individual, eg. by quantifying the amount of a biomarker in a sample or samples derived from a single individual. Alternatively, the reference value may be derived by pooling data obtained from two or more (e.g., at least three, four, five, 10, 15, 20 or 25) individuals (ie. a population of individuals) and calculating an average (for example, mean or median) amount for a biomarker. Thus, the reference value may reflect the average amount of a biomarker in a given population of individuals. Said amounts may be expressed in absolute or relative terms, in the same manner as described above in relation to the sample that is to be tested using the method of the invention. As used herein, the term “population of individuals” refers to a group of two or more individuals, such as at least three, four, five, 10, 15, 20 or 25 individuals.

When comparing between the sample and the reference value, the way in which the amounts are expressed is matched between the sample and the reference value. Thus, an absolute amount can be compared with an absolute amount, and a relative amount can be compared with a relative amount.

The reference value may be derived from the same sample as the sample that is being tested, thus allowing for an appropriate comparison between the two. Thus, by way of example, if the sample is derived from a blood sample, then the reference value will also be a blood sample.

When the amounts of two or more biomarkers are determined, the amount of each biomarker may be compared to its corresponding reference value. Alternatively, when the cumulative amount of all the biomarkers is determined, the cumulative amount the biomarkers may be compared to a cumulative corresponding reference value. Alternatively, when the amount of the two or more biomarkers are combined with each other in a formula to form an index value, the index value can be compared to a corresponding reference index value derived in the same manner.

The reference values may be obtained either within (ie. constituting a step of) or separately to (ie. not constituting a step of) the methods of the invention. In one embodiment, the methods of the invention may comprise a step of establishing a reference value for the quantity of the markers. In one embodiment, the reference values are obtained separately to the method of the invention and accessed (eg. on a database) during the comparison step of the invention.

As illustrated in FIG. 1, the present inventors observed that all of biomarkers shown in Table 1 increased in abundance in samples obtained from patients having a systemic inflammatory condition (such as sepsis or SIRS), as compared to healthy individuals. Detecting elevated levels of one or more of these biomarkers in a patient can thus be used to diagnose the presence of a systemic inflammatory condition in a patient. In particular, the differences in marker abundance between individuals having a systemic inflammatory condition and individuals that are healthy provides a way to classify individuals as having a systemic inflammatory condition or not having a systemic inflammatory condition by determining their marker profile.

By comparing the presence and/or amount of markers quantified in a sample obtained from a test patient to the presence and/or amount of markers quantified for a reference value (such as that obtained from a population of healthy individuals, or from a population of individuals having sepsis (eg. abdominal sepsis or pulmonary sepsis) or SIRS), it is possible to diagnose whether the patient has a systemic inflammatory condition (such as abdominal sepsis, pulmonary sepsis or SIRS). The method permits classification of the patient as belonging to or not belonging to the reference population (ie. by determining whether the amounts of marker quantified in the patient are statistically similar to the reference population or statistically deviate from the reference population). Hence, classification of the patient's marker profile (i.e., the overall pattern of change observed for the markers quantified) as corresponding to the profile derived from a particular reference population is predictive that the patient falls (or does not fall) within the reference population.

In one embodiment, a patient may be diagnosed as having or being at risk of having a systemic inflammatory condition (such as sepsis (eg. abdominal sepsis or pulmonary sepsis) or SIRS), when the amount of the one or more biomarker is statistically similar to the amount determined for the corresponding reference value representative of an individual (or a population of individuals) having sepsis (eg. abdominal sepsis or pulmonary sepsis) and/or the amount determined for the corresponding reference value representative of an individual (or a population of individuals) having SIRS. In one embodiment, a patient may be diagnosed as not having or not being at risk of having a systemic inflammatory condition (such as sepsis (eg. abdominal sepsis or pulmonary sepsis) or SIRS) when the amount of the one or more biomarker is statistically similar to the amount determined for the corresponding reference value representative of a healthy individual (or a population of healthy individuals).

As used herein, the term “statistically similar” means that the amount of the one or more biomarker quantified for the test patient is similar to the amount quantified for the reference population to a statistically significant level. The term “statistically significant” means that the alteration is greater than what might be expected to happen by chance alone (p=<0.05). Statistical significance can be determined by any method known in the art.

In one embodiment, a patient may be diagnosed as having or being at risk of having a systemic inflammatory condition (such as sepsis (eg. abdominal sepsis or pulmonary sepsis) or SIRS) when the amount of the one or more biomarker statistically deviates from the amount determined for the corresponding reference value representative of a healthy individual (or a population of healthy individuals). In one embodiment, a patient may be diagnosed as not having or not being at risk of having a systemic inflammatory condition (such as sepsis (eg. abdominal sepsis or pulmonary sepsis) or SIRS) when the amount of the one or more biomarker statistically deviates from the amount determined for the corresponding reference value representative of an individual (or a population of individuals) having sepsis (eg. abdominal sepsis or pulmonary sepsis) and/or an individual (or a population of individuals) having SIRS.

As used herein, the term “statistically deviates” means that the amount of the one or more biomarker quantified for the test patient differs from the amount quantified for the reference population to a statistically significant level. The term “statistically significant” means that the alteration is greater than what might be expected to happen by chance alone (p=<0.05). Statistical significance can be determined by any method known in the art. The deviation in biomarker abundance may be an increase or decrease. The increase or decrease may be statistically significant.

The amount of the one or more biomarker of the invention, for example in a biomarker profile, may differ by at least 10%, at least 20%, at least 30%, at least 40%, at least 50%, at least 60, at least 70%, at least 80%, at least 90%, at least 100%, at least 150%, at least 200% or more compared with a corresponding reference value.

The amount of the one or more biomarker of the invention, for example in a biomarker profile, may differ by at least 0.1, at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, or at least 50 fold as compared to a corresponding reference value.

For example, if the amount of the one or more biomarker of the invention, typically in a biomarker profile, is reduced compared with a corresponding reference value, the expression may be reduced partially or totally compared with the corresponding reference value. Typically, the amount is reduced by at least 10%, at least 20%, at least 30%, at least 40%, at least 50%, at least 60, at least 70%, at least 80%, at least 90%, at least 95%, at least 99%, up to total elimination of the one or more biomarker. Typically the amount is reduced by at least 0.1, at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, or at least 50 fold as compared to a corresponding reference value. For example, the fold decrease may be at least 0.5 fold. For example, the fold decrease may be at least 1 fold. For example, the fold decrease may be at least 1.5 fold. For example, the fold decrease may be at least 2 fold. For example, the fold decrease may be at least 2.5 fold. For example, the fold decrease may be at least 3 fold. For example, the fold decrease may be at least 3.5 fold. For example, the fold decrease may be at least 4 fold. For example, the fold decrease may be at least 4.5 fold. For example, the fold decrease may be at least 5 fold. The decrease in the amount of the marker may be statistically significant.*

For example, if the amount of one or more biomarker of the invention, typically in a biomarker profile, is increased compared with a corresponding reference value, the amount may be increased by at least 10%, at least 20%, at least 30%, at least 40%, at least 50%, at least 60, at least 70%, at least 80%, at least 90&, at least 100%, at least 150%, at least 200% compared with the corresponding reference value. The amount may be increased by at least 0.1, at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, or at least 50 fold as compared to a corresponding reference value. For example, the fold increase may be at least 0.5 fold. For example, the fold increase may be at least 1 fold. For example, the fold increase may be at least 1.5 fold. For example, the fold increase may be at least 2 fold. For example, the fold increase may be at least 2.5 fold. For example, the fold increase may be at least 3 fold. For example, the fold increase may be at least 3.5 fold. For example, the fold increase may be at least 4 fold. For example, the fold increase may be at least 4.5 fold. For example, the fold increase may be at least 5 fold. The increase in the amount of the marker may be statistically significant.

The amount of the one or more biomarker may be altered compared with a corresponding reference value for at least 12 hours, at least 24 hours, at least 30 hours, at least 48 hours, at least 72 hours, at least 96 hours, at least 120 hours, at least 144 hours, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or more.

As described herein, the present inventors observed that all of biomarkers shown in Table 1 increased in abundance in samples obtained from patients having a systemic inflammatory condition, as compared to healthy individuals. Detecting elevated levels of one or more of these biomarkers in a patient can thus be used to diagnose the presence of a systemic inflammatory condition in a patient.

Thus, in one embodiment, when the reference value is representative of a healthy individual (or population of healthy individuals), an increase in the one or more biomarker in the sample obtained from the patient relative to the corresponding reference value, indicates that the patient has or is at risk of having a systemic inflammatory condition. Likewise, no increase in the one or more biomarker in the sample obtained from the patient relative to the corresponding reference value, indicates that the patient does not have a systemic inflammatory condition.

The present inventors observed that the overall increase in biomarker abundance observed in patients having a systemic inflammatory condition varied between different biomarkers, with some biomarkers showing very significant increases in abundance, and others showing more subtle changes.

In one embodiment, the patient may be diagnosed as having a systemic inflammatory condition, or being at risk of developing a systemic inflammatory condition, when the one or more biomarker (or the one or more additional biomarker) increases by at least 0.1 (e.g. at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value representative of a healthy individual.

The ‘comparison’ step of the methods of the invention may comprise applying a decision rule, or using a decision tree. A “decision rule” or a “decision tree” is a method used to classify individuals. This rule can take on one or more forms that are known in the art, as exemplified in Hastie et al., in “The Elements of Statistical Learning” Springer-Nerlag (Springer, New York (2001)). Analysis of biomarkers in the complex mixture of molecules within the sample generates features in a data set. A decision rule or a decision tree may be used to act on a data set of features to diagnose, monitor, or prognose a systemic inflammatory condition (such as sepsis or SIRS), to distinguish between sepsis and SIRS in a patient, or to distinguish between abdominal sepsis and pulmonary sepsis.

The decision rule or decision tree can comprise a data analysis algorithm, such as a computer pattern recognition algorithm. Other suitable algorithms include, but are not limited to, logistic regression or a nonparametric algorithm that detects differences in the distribution of feature values (e.g., a Wilcoxon Signed Rank Test). The decision rule may be based upon one, two, three, four, five, 10, 20 or more features. In one embodiment, the decision rule or decision tree is based on hundreds or more of features. Applying the decision rule or decision tree may also comprise using a classification tree algorithm. For example, the reference value (or reference biomarker profile) may comprise at least three features or biomarkers, where the features are predictors in a classification tree algorithm. The data analysis algorithm predicts membership within a population (or class) with an accuracy of at least about 60%, at least about 70%, at least about 80% and at least about 90%.

Suitable algorithms are known in the art, some of which are reviewed in Hastie et al, supra. Such algorithms classify complex spectra from biological materials, such as a blood sample, to distinguish individuals as normal or as possessing biomarker expression levels characteristic of a particular disease state. While such algorithms may be used to increase the speed and efficiency of the application of the decision rule and to avoid investigator bias, one of ordinary skill in the art will realise that computer-based algorithms are not required to carry out the methods of the present invention.

Algorithms may be applied to the comparison of the one or more biomarker or the biomarker profiles, regardless of the method that was used to generate the data for the one or more biomarker or the biomarker profile. For example, suitable algorithms can be applied to biomarker profiles generated using gas chromatography, as discussed in Harper, “Pyrolysis and GC in Polymer Analysis” Dekker, New York (1985). Further, Wagner et al, Anal Chem 74: 1824-35 (2002) disclose an algorithm that improves the ability to classify individuals based on spectra obtained by static time-of-flight secondary ion mass spectrometry (TOF-SIMS). Additionally, Bright et al, J. Microbiol Methods 48: 127-38 (2002) disclose a method of distinguishing between bacterial strains with high certainty (79-89% correct classification rates) by analysis of MALDI-TOF-MS spectra. Dalluge, Fresenius J. Anal. Chem. 366: 701-11 (2000) discusses the use of MALDI-TOF-MS and liquid chromatography-electrospray ionization mass spectrometry (LC/ESI-MS) to classify profiles of biomarkers in complex biological samples.

The methods and uses of the invention may thus comprise applying a decision rule as described herein. Applying the decision rule may comprise using a data analysis algorithm, also as described herein. The data analysis algorithm may comprise at least two, at least three, at least four, at least five, at least six, at least seven, at least eight, at least nine, at least ten, at least 15, at least 20, at least 25, at least 50 or more input parameters. The data analysis algorithm may use any of the biomarkers of the invention, or combination of biomarkers of the invention as input parameters. Typically, the data analysis algorithm uses at least two, at least three, at least four, at least five, at least six, at least seven, at least eight, at least nine, at least ten, at least 15, at least 20, at least 25, at least 50 of the biomarkers of the invention (e.g. as listed in any one of Tables 1 to 4) as input parameters.

The application of the decision rule or the decision tree does not require perfect classification. A classification may be made with at least about 90% certainty, or even more, in one embodiment. In other embodiments, the certainty is at least about 80%, at least about 70%, or at least about 60%. The useful degree of certainty may vary, depending on the particular method of the present invention. “Certainty” is defined as the total number of accurately classified individuals divided by the total number of individuals subjected to classification. As used herein, “certainty” means “accuracy”.

Classification may also be characterized by its “sensitivity”. The “sensitivity” of classification relates to the percentage of individuals who were correctly identified as having a particular disease or condition eg. the percentage of individuals who were correctly identified as having a systemic inflammatory condition (such as sepsis or SIRS). “Sensitivity” is defined in the art as the number of true positives divided by the sum of true positives and false negatives.

The “specificity” of a method is defined as the percentage of patients who were correctly identified as not having particular disease or condition, eg. the percentage of individuals who were correctly identified as not having a systemic inflammatory condition (such as sepsis or SIRS). That is, “specificity” relates to the number of true negatives divided by the sum of true negatives and false positives.

Typically, the accuracy, sensitivity and/or specificity of the methods and uses of the invention is at least about 90%, at least about 80%, at least about 70% or at least about 60%.

The method for diagnosing a systemic inflammatory condition in a patient as described herein can be used in a decision tree process to investigate the health of a patient having or suspected of having a systemic inflammatory condition. For example, the method for diagnosing a systemic inflammatory condition in a patient can be performed before, after, or in addition to any of the other methods described herein.

In one embodiment, the method for diagnosing a systemic inflammatory condition in a patient is performed as described herein. If the patient tests positive for a systemic inflammatory condition, they may be tested using the method for distinguishing between sepsis and SIRS described herein to determine whether the patient has sepsis and/or SIRS. In one embodiment, the above combination of methods are performed as described, and if the patient tests positive for sepsis, the patient may be further tested for sepsis, abdominal sepsis and/or pulmonary sepsis using the diagnostic methods described herein, so as to confirm whether the patient has or is at risk of developing sepsis, and/or determine whether the patient has or is at risk of developing abdominal sepsis and/or pulmonary sepsis. If the patient tests positive for SIRS, the patient may be further tested for SIRS using the diagnostic method described herein, so as to confirm whether the patient has or is at risk of developing SIRS. If the patient tests positive for sepsis using the method for diagnosis of sepsis (as described herein), the patient may be further tested for abdominal sepsis and/or pulmonary sepsis using the methods described herein.

In one embodiment, the method of the invention for diagnosing a systemic inflammatory condition in a patient is performed as described herein. If the patient tests positive for a systemic inflammatory condition, they may be tested for sepsis, abdominal sepsis, pulmonary sepsis and/or SIRS using the diagnostic methods described herein. The methods for diagnosis of sepsis, abdominal sepsis, pulmonary sepsis and/or SIRS may be performed simultaneously or sequentially in any order.

The above described combination of methods may also be performed in parallel to determine the disease status of a patient by simultaneously (or substantially simultaneously) investigating the expression of all the biomarkers in a sample obtained from the patient, and determining whether the patient has or is at risk of having a systemic inflammatory condition, sepsis (such as abdominal or pulmonary sepsis) and/or SIRS.

When performing these different methods in a decision tree process, the sample used in each step of the method may be the same sample obtained from the patient. When the method comprises multiple quantification steps, these multiple steps may be performed at the same time (e.g. in parallel) and/or using the same sample. When the method comprises multiple comparison steps, these multiple steps may be performed at the same time (e.g. in parallel).

In all methods described herein, any appropriate technique may be used to confirm the diagnosis. Standard techniques are known in the art. For example, confirmation of a diagnosis of a systemic inflammatory condition in a patient may include: testing for the presence of other known biomarkers of a systemic inflammatory condition including: C-reactive protein (CRP), Procalcitonin (PCT), lactate, Cystatin C (CYTC), Neutrophil gelatinase-associated lipocalin (NGAL) and interleukin 6 (IL6).

Additional clinical parameters that may be used to confirm the diagnosis also include: white blood cell count, kidney function tests (such as serum creatinine, or urine output), respiratory system function tests (such as PaO2/FiO2), nervous system function tests (expressed as Glasgow coma scale), cardiovascular function tests (expressed as mean arterial pressure), liver function tests (such as bilirubin concentration), and coagulation function tests (such as platelet concentration). The methods and uses of the invention may further comprise determining such clinical parameters in the patient.

In a related aspect, the present invention also provides the use of one or more of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1 as a biomarker for a systemic inflammatory condition. For example, the one or more biomarker may be selected from: FAM20A, OLAH and/or CD177.

In one embodiment, the use is of the one or more biomarker in the diagnosis of a systemic inflammatory condition in a patient. For example, the use may comprise (i) determining the presence and/or amount of one or more biomarker in a sample obtained from a patient; and (ii) comparing the presence and/or amount of the one or more biomarker determined in said sample in (i) to a corresponding reference value to determine whether the patient has a systemic inflammatory condition.

All embodiments described above for the method of diagnosing a systemic inflammatory condition in a patient apply equally to the ‘use’ of the invention described herein. This includes all embodiments relating to the terms “systemic inflammatory condition”, “patient”, “sample”, and “the one or more biomarker”.

### Diagnosis of SIRS

When investigating gene expression patterns in patients having systemic inflammatory conditions, the inventors observed that certain biomarkers (see Table 2) were particularly elevated in abundance in patients having SIRS, as compared to patients having other systemic inflammatory conditions, and healthy individuals. As a result of these findings, the inventors thus observed that PLA2G7, ARHGEF10L, MYCL, TGFBI, GPR124, URN, NLRP3, RBP4, and MPP3 can be used as biomarkers for diagnosis of SIRS.

The present invention therefore provides a method for diagnosing SIRS in a patient, comprising:

(i) determining the presence and/or amount of one or more biomarker in a sample obtained from a patient, wherein the one or more biomarker is selected from the group consisting of: PLA2G7, ARHGEF10L, MYCL, TGFBI, GPR124, IL1 RN, NLRP3, RBP4, and MPP3;
(ii) comparing the presence and/or amount of the one or more biomarker determined in said sample in (i) to a corresponding reference value; and thereby determining whether the patient has or is at risk of developing SIRS.

All embodiments described above for the “method for diagnosing a systemic inflammatory condition in a patient” apply equally to the “method for diagnosing SIRS in a patient”. This includes all embodiments relating to the “sample”, “patient”, “biomarker”, and “reference value”, and all embodiments relating to the quantification step for “determining the presence and/or amount of one or more biomarker in a sample” and the “comparison” step used to make a conclusion about the disease state of the patient.

As used herein, the phrase “diagnosis of SIRS in a patient” means determining whether the patient has or is risk of developing SIRS. The systemic inflammatory condition “SIRS” diagnosed using the method of the invention is as described above for the “method for diagnosing a systemic inflammatory condition in a patient”.

The “patient” for which diagnosis is performed is as described above for the “method for diagnosing a systemic inflammatory condition in a patient”. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having a systemic inflammatory condition using the method described herein. In one embodiment, the patient has been diagnosed as having or being at risk of developing SIRS and/or sepsis using the method of the invention for distinguishing between sepsis and SIRS in a patient as described herein. In one embodiment, the patient is suspected of having or being at risk of developing SIRS.

The “sample” obtained from the patient is as described above for the “method for diagnosing a systemic inflammatory condition in a patient”, including all embodiments relating to the time point at which the sample is obtained.

The “one or more biomarker” of the invention is as described above for the “method for diagnosing a systemic inflammatory condition in a patient”.

As illustrated in Example 1, the present inventors observed that PLA2G7, ARHGEF10L, MYCL, TGFBI, GPR124, IL1 RN, NLRP3, RBP4, and MPP3 are elevated in patients having SIRS, and can thus be used as biomarkers for diagnosis of SIRS.

The reference to the biomarker MYCL throughout the entire description, includes the transcript variant 1 of MYCL (as encoded by SEQ ID NO: 37) and the transcript variant 3 of MYCL (as encoded by SEQ ID NO: 38). In one embodiment, the reference to the biomarker MYCL is a reference to the transcript variant 1 of MYCL (as encoded by SEQ ID NO: 37). In one embodiment, the reference to the biomarker MYCL is a reference to the transcript variant 3 of MYCL (as encoded by SEQ ID NO: 38).

In one embodiment, the one or more biomarker may be selected from the group consisting of: PLA2G7, ARHGEF10L, MYCL, TGFBI, GPR124, IL1 RN, NLRP3, RBP4, and MPP3. For example, the one or more biomarker may be selected from the group consisting of: PLA2G7, ARHGEF10L, MYCL, TGFBI, GPR124, RBP4, and MPP3. For example, the one or more biomarker may be selected from the group consisting of: MYCL, TGFBI, GPR124, NLRP3, and MPP3. For example, the one or more biomarker may be selected from the group consisting of: ARHGEF10L, TGFBI, GPR124, IL1RN, NLRP3, RBP4, and MPP3. For example, the one or more biomarker may be selected from the group consisting of: TGFBI, GPR124, URN, NLRP3, RBP4, and MPP3. For example, the one or more biomarker may be selected from the group consisting of: GPR124, URN, NLRP3, RBP4, and MPP3. For example, the one or more biomarker may be selected from the group consisting of: GPR124, NLRP3, and MPP3.

The present inventors observed that a sub-set of the biomarkers for SIRS (GPR124, TGFBI, PLA2G7, MYCL, and ARHGEF10L) increase in abundance in patients having SIRS compared to healthy individuals, but do not increase in abundance in patients having sepsis as compared to healthy individuals. These markers therefore provide highly specific biomarkers for diagnosing SIRS. Thus, in one embodiment, the one or more biomarker may be selected from the group consisting of: PLA2G7, ARHGEF10L, MYCL, TGFBI, and GPR124. For example, the one or more biomarker may be selected from the group consisting of: PLA2G7, ARHGEF10L, MYCL, and TGFBI.

Each of the biomarkers of SIRS may be used alone, or in combination with any of the SIRS biomarkers in the method of the invention. For example, any combination of 1 or more, 2 or more, 3 or more, 4 or more, 5 or more, 6 or more, 7 or more, 8 or more, up to and including all of the SIRS biomarkers may be used to diagnose SIRS in a patient according to the method of the invention.

In one embodiment, any combination of 1 or more (eg. 2 or more, 3 or more, 4 or more, 5 or more, 6 or more, 7 or more, 8 or more, or all 9) of the biomarkers selected from the group consisting of: PLA2G7, ARHGEF10L, MYCL, TGFBI, GPR124, URN, NLRP3, RBP4, and MPP3, may be used to diagnose SIRS in a patient. In one embodiment, any combination of 1 or more (eg. 2 or more, 3 or more, 4 or more, or all 5) of the biomarkers selected from the group consisting of: PLA2G7, ARHGEF10L, MYCL, TGFBI, and GPR124, may be used to diagnose SIRS in a patient. For example, any combination of 1 or more (eg. 2 or more, 3 or more, or all 4) of the biomarkers selected from the group consisting of: ARHGEF10L, MYCL, TGFBI, and GPR124 may be used to diagnose SIRS in a patient. For example, any combination of 1 or more (eg. 2 or more, 3 or more, or all 4) of the biomarkers selected from the group consisting of: ARHGEF10L, MYCL, TGFBI, and GPR124, may be used to diagnose SIRS in a patient. For example, any combination of 1 or more (eg. 2 or more, or all 3) of the biomarkers selected from the group consisting of: MYCL, TGFBI and GPR124, may be used to diagnose SIRS in a patient. For example, any combination of 1 or more (eg. 2 or more, or all 3) of the biomarkers selected from the group consisting of: PLA2G7, TGFBI and GPR124, may be used to diagnose SIRS in a patient. For example, any combination of 1 or more (or both) of the biomarkers selected from the group consisting of: MYCL and GPR124, may be used to diagnose SIRS in a patient. For example, any combination of 1 or more (or both) of the biomarkers selected from the group consisting of: PLA2G7 and GPR124, may be used to diagnose SIRS in a patient.

In a further example, the following combinations of SIRS biomarkers may be used in the method of the invention to diagnose SIRS: (i) TGFBI and PLA2G7; (ii) TGFBI and GPR124; (iii) TGFBI and MYCL; (iv) TGFBI and ARHGEF10L; (v) PLA2G7 and GPR124; (vi) PLA2G7 and MYCL; (vii) PLA2G7 and ARHGEF10L; (viii) GPR124 and MYCL; (ix) GPR124 and ARHGEF10L; (x) MYCL and ARHGEF10L.

As described in Example 2, a subset of the SIRS biomarkers (PLA2G7, ARHGEF10L, MYCL, and TGFBI) were shown to be particularly effective in diagnosing SIRS when tested by ROC analysis. Specifically, AUC values of 0.89, 0.8, 0.8, 0.79 were achieved for PLA2G7, ARHGEF10L, MYCL, and TGFBI. Thus, in one embodiment, any combination of 1 or more (eg. 2 or more, 3 or more, or all 4) of the biomarkers selected from the group consisting of: PLA2G7, ARHGEF10L, MYCL, and TGFBI, may be used to diagnose SIRS in a patient. For example, 2 or more of the biomarkers selected from the group consisting of: PLA2G7, ARHGEF10L, MYCL, and TGFBI, may be used to diagnose SIRS in a patient. For example, 3 or more of the biomarkers selected from the group consisting of: PLA2G7, ARHGEF10L, MYCL, and TGFBI, may be used to diagnose SIRS in a patient. In one embodiment, the method of the invention may be preferably performed using the combination of: PLA2G7, ARHGEF10L, MYCL, and TGFBI.

In one embodiment, the one or more biomarker is TGFBI. In one embodiment, the one or more biomarker is PLA2G7. In one embodiment, the one or more biomarker is MYCL. In one embodiment, the one or more biomarker is ARHGEF10L. In one embodiment, the one or more biomarker is GPR124. In one embodiment, the one or more biomarker is URN. In one embodiment, the one or more biomarker is NLRP3. In one embodiment, the one or more biomarker is RBP4. In one embodiment, the one or more biomarker is MPP3.

One or more additional biomarker for SIRS may also be used in the diagnosis of SIRS according to the method of the invention. Any combination of the one or more additional biomarker may be used in combination with the one or more biomarker of the invention. For example at least 1, at least 2, at least 3, at least 4, at least 5, at least 6, at least 7, or all 8 additional biomarkers for SIRS may be used in combination with the one or more biomarker of the invention (as described herein). Typically, the one or more additional biomarker is selected from the group consisting of: PLA2G7, ARHGEF10L, MYCL, TGFBI, GPR124, URN, NLRP3, RBP4, and MPP3. For example, the one or more additional biomarker is selected from the group consisting of: PLA2G7, ARHGEF10L, MYCL, TGFBI and GPR124.

In one embodiment, the one or more biomarker is TGFBI, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, GPR124, IL1 RN, NLRP3, RBP4, and MPP3. In one embodiment, the one or more additional biomarker is selected from at least 1 (eg. at least 2, at least 3, up to and including all) of the biomarkers: PLA2G7, ARHGEF10L, MYCL, and GPR124. In one embodiment, the one or more additional biomarker is selected from at least 1 (eg. at least 2, up to and including all) of the biomarkers: PLA2G7, ARHGEF10L and MYCL.

In one embodiment, the one or more biomarker is PLA2G7, and the one or more additional biomarker is selected from at least 1 (eg. at least 2, at least 3, at least 4, at least 5, at least 6, at least 7, up to and including all) of the biomarkers: ARHGEF10L, MYCL, TGFBI, GPR124, URN, NLRP3, RBP4, and MPP3. In one embodiment, the one or more additional biomarker is selected from at least 1 (eg. at least 2, at least 3, up to and including all) of the biomarkers: ARHGEF10L, MYCL, TGFBI, and GPR124. In one embodiment, the one or more additional biomarker is selected from at least 1 (eg. at least 2, up to and including all) of the biomarkers: ARHGEF10L, MYCL and TGFBI.

In one embodiment, the one or more biomarker is MYCL, and the one or more additional biomarker is selected from at least 1 (eg. at least 2, at least 3, at least 4, at least 5, at least 6, at least 7, up to and including all) of the biomarkers: PLA2G7, ARHGEF10L, TGFBI, GPR124, IL1 RN, NLRP3, RBP4, and MPP3. In one embodiment, the one or more additional biomarker is selected from at least 1 (eg. at least 2, at least 3, up to and including all) of the biomarkers: PLA2G7, ARHGEF10L, TGFBI and GPR124. In one embodiment, the one or more additional biomarker is selected from at least 1 (eg. at least 2, up to and including all) of the biomarkers: PLA2G7, ARHGEF10L and TGFBI.

In one embodiment, the one or more biomarker is ARHGEF10L, and the one or more additional biomarker is selected from at least 1 (eg. at least 2, at least 3, at least 4, at least 5, at least 6, at least 7, up to and including all) of the biomarkers: PLA2G7, MYCL, TGFBI, GPR124, IL1 RN, NLRP3, RBP4, and MPP3. In one embodiment, the one or more additional biomarker is selected from at least 1 (eg. at least 2, at least 3, up to and including all) of the biomarkers: PLA2G7, MYCL, TGFBI, and GPR124. In one embodiment, the one or more additional biomarker is selected from at least 1 (eg. at least 2, up to and including all) of the biomarkers: PLA2G7, MYCL and TGFBI.

In one embodiment, the one or more biomarker is GPR124, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, TGFBI, URN, NLRP3, RBP4, and MPP3. In one embodiment, the one or more additional biomarker is selected from at least 1 (eg. at least 2, at least 3, up to and including all) of the biomarkers: PLA2G7, ARHGEF10L, MYCL, and TGFBI.

In one embodiment, the one or more biomarker is URN, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, TGFBI, GPR124, NLRP3, RBP4, and MPP3. In one embodiment, the one or more additional biomarker is selected from at least 1 (eg. at least 2, at least 3, at least 4, up to and including all) of the biomarkers: PLA2G7, ARHGEF10L, MYCL, TGFBI, and GPR124.

In one embodiment, the one or more biomarker is NLRP3, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, TGFBI, GPR124, URN, RBP4, and MPP3. In one embodiment, the one or more additional biomarker is selected from at least 1 (eg. at least 2, at least 3, at least 4, up to and including all) of the biomarkers: PLA2G7, ARHGEF10L, MYCL, TGFBI, and GPR124.

In one embodiment, the one or more biomarker is RBP4, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, TGFBI, GPR124, IL1RN, NLRP3, and MPP3. In one embodiment, the one or more additional biomarker is selected from at least 1 (eg. at least 2, at least 3, at least 4, up to and including all) of the biomarkers: PLA2G7, ARHGEF10L, MYCL, TGFBI, and GPR124.

In one embodiment, the one or more biomarker is MPP3, and the one or more additional biomarker is selected from at least 1 (eg. at least 2, at least 3, at least 4, at least 5, at least 6, at least 7, up to and including all) of the biomarkers: PLA2G7, ARHGEF10L, MYCL, TGFBI, GPR124, IL1RN, NLRP3, and RBP4. In one embodiment, the one or more additional biomarker is selected from at least 1 (eg. at least 2, at least 3, at least 4, up to and including all) of the biomarkers: PLA2G7, ARHGEF10L, MYCL, TGFBI, and GPR124.

As illustrated in FIG. 2, the present inventors observed that the “SIRS” biomarkers described herein increase in abundance in patients having SIRS as compared to patients having other systemic inflammatory conditions (such as abdominal sepsis or pulmonary sepsis), and as compared to healthy individuals. These differences in marker abundance can be used to diagnose whether an individual has or is at risk of developing SIRS.

For example, by comparing the presence and/or amount of markers quantified in a sample obtained from a patient to the presence and/or amount of markers quantified for a reference value (such as a reference value that is representative of a healthy individual (or a population of healthy individuals), a reference value that is representative of an individual (or a population of individuals) having sepsis (eg. abdominal and/or pulmonary sepsis), or a reference value that is representative of an individual (or a population of individuals) having SIRS, it is possible to diagnose the presence (or absence) of SIRS in a patient. The method permits classification of the patient as belonging to or not belonging to the reference population (ie. by determining whether the amounts of marker quantified in the individual are statistically similar to the reference population or statistically deviate from the reference population). Hence, classification of the patient's marker profile (ie. the overall pattern of change observed for the markers quantified) as corresponding to the profile derived from a particular reference population is predictive that the patient falls (or does not fall) within the reference population.

In one embodiment, a patient may be diagnosed as having or being at risk of having SIRS, when the amount of the one or more biomarker is statistically similar to the amount determined for the corresponding reference value representative of an individual (or a population of individuals) having SIRS. In one embodiment, a patient may be diagnosed as not having or not being at risk of having SIRS when the amount of the one or more biomarker is statistically similar to the amount determined for the corresponding reference value representative of a healthy individual (or a population of healthy individuals). In one embodiment, a patient may be diagnosed as not having or not being at risk of having SIRS when the amount of the one or more biomarker quantified is statistically similar to the amount determined for the corresponding reference value representative of an individual having sepsis (or a population of individuals having sepsis).

In one embodiment, a patient may be diagnosed as having or being at risk of having SIRS when the amount of the one or more biomarker statistically deviates from the amount determined for the corresponding reference value representative of a healthy individual (or a population of healthy individuals). In one embodiment, a patient may be diagnosed as having or being at risk of having SIRS when the amount of the one or more biomarker statistically deviates from the amount determined for the corresponding reference value representative of an individual having sepsis (or a population of individuals having sepsis). In one embodiment, a patient may be diagnosed as not having or not being at risk of having SIRS when the amount of the one or more biomarker statistically deviates from the amount determined for the corresponding reference value representative of an individual (or a population of individuals) having SIRS.

All embodiments described above for the classification of a patient as having or being at risk of having a systemic inflammatory condition (or as not having or not being at risk of having a systemic inflammatory condition) apply equally to the method for diagnosing whether a patient has or is at risk of having SIRS. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may as defined above for the method of diagnosing a systemic inflammatory condition in a patient. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having sepsis (or a population of individuals having sepsis).

As described above for the “method for diagnosing a systemic inflammatory condition in a patient”, the method of the invention may involve the use of multiple separate reference values. For example, the reference value may include one of more (eg. two or more, or all 3) of the reference values selected from: a reference value that is representative of a healthy individual (or a population of healthy individuals); a reference value that is representative of an individual having SIRS (or a population of individuals having SIRS); and a reference value that is representative of an individual having sepsis (or a population of individuals having sepsis).

The present inventors observed that the SIRS biomarkers described herein each increase in abundance in samples obtained from patients having SIRS, as compared to healthy individuals. Detection of increased levels of these biomarkers in a patient as compared to the levels detected for healthy individuals can thus be used to diagnose the presence of SIRS. In one embodiment, when the reference value is representative of a healthy individual (or population of healthy individuals), an increase in the one or more biomarker (and/or one or more additional biomarker) in the sample obtained from the patient relative to the corresponding reference value indicates that the patient has or is at risk of developing SIRS. Likewise, no increase in the one or more biomarker (and/or one or more additional biomarker) in the sample obtained from the patient relative to the corresponding reference value indicates that the patient does not have SIRS.

For some of the SIRS biomarkers identified by the present inventors, increased levels of these markers were also observed in patients having sepsis as compared to healthy individuals, although much bigger increases were observed for these biomarkers in the patients having SIRS. The accuracy of SIRS diagnosis can thus be improved by looking for a “minimum” fold change or % change in the levels of the one or more biomarkers as compared to the corresponding reference value that is representative of a healthy individual. The fold change or % change may be as defined above for the method for diagnosis of a systemic inflammatory condition.

In one embodiment, the patient may be diagnosed as having SIRS, or being at risk of developing SIRS, when the one or more biomarker (or the one or more additional biomarker) increases by at least 0.1 (e.g. at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value representative of a healthy individual.

For example, an increase of at least 1.1 (eg. at least 1.2, at least 1.3, at least 1.4, at least 1.5) fold in GPR124 in the sample obtained from the patient relative to the corresponding reference value, indicates that the patient has, or is at risk of developing SIRS. In one embodiment, no increase or an increase of less than 1.1 (eg. less than 1.2, less than 1.3, less than 1.4, less than 1.5, less than 1.6) fold in GPR124 in the sample obtained from the patient relative to the corresponding reference value, indicates that the patient does not have, or is not at risk of developing SIRS.

For example, an increase of at least 1.5 (eg. at least 1.6, at least 1.7, at least 1.8, at least 1.9, or at least 2) fold in TGFBI in the sample obtained from the patient relative to the corresponding reference value, indicates that the patient has, or is at risk of developing SIRS. In one embodiment, no increase or an increase of less than 1.5 (eg. less than 1.6, less than 1.7, or less than 1.8) fold in TGFBI in the sample obtained from the patient relative to the corresponding reference value, indicates that the patient does not have, or is not at risk of developing SIRS.

For example, an increase of at least 1.1 (eg. at least 1.2, at least 1.3, at least 1.4, at least 1.5, or at least 1.6) fold in PLA2G7 in the sample obtained from the patient relative to the corresponding reference value, indicates that the patient has, or is at risk of developing SIRS. In one embodiment, no increase or an increase of less than 1.1 (eg. less than 1.2, less than 1.3, less than 1.4, less than 1.5, or less than 1.6) fold in PLA2G7 in the sample obtained from the patient relative to the corresponding reference value, indicates that the patient does not have, or is not at risk of developing SIRS. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.1 (eg. at least 1.2, at least 1.3, or at least 1.4) fold in MYCL in the sample obtained from the patient relative to the corresponding reference value, indicates that the patient has, or is at risk of developing SIRS. In one embodiment, no increase or an increase of less than 1.1 (eg. less than 1.2, or less than 1.3, or less than 1.4) fold in MYCL in the sample obtained from the patient relative to the corresponding reference value, indicates that the patient does not have, or is not at risk of developing SIRS. In one embodiment, when detecting this biomarker, the method is performed using a sample obtained from a patient at least 24 (eg. at least 36, at least 48, at least 72, at least 96, or at least 120) hours after the patient presents with one or more clinical symptoms of a systemic inflammatory condition, or is admitted to a medical care facility.

For example, an increase of at least 1.1 (eg. at least 1.2, at least 1.3, at least 1.4, at least 1.5) fold in ARHGEF10L in the sample obtained from the patient relative to the corresponding reference value, indicates that the patient has, or is at risk of developing SIRS. In one embodiment, no increase or an increase of less than 1.1 (eg. less than 1.2, less than 1.3, less than 1.4, or less than 1.5) fold in ARHGEF10L in the sample obtained from the patient relative to the corresponding reference value, indicates that the patient does not have, or is not at risk of developing SIRS.

For example, an increase of at least 4 (eg. at least 4.1, at least 4.2, at least 4.3, at least 4.4, or at least 4.5) fold in IL1 RN in the sample obtained from the patient relative to the corresponding reference value, indicates that the patient has, or is at risk of developing SIRS. In one embodiment, no increase or an increase of less than 4 (eg. less than 4.1, less than 4.2, less than 4.3, less than 4.4, or less than 4.5) fold in IL1RN in the sample obtained from the patient relative to the corresponding reference value, indicates that the patient does not have, or is not at risk of developing SIRS.

For example, an increase of at least 2 (eg. at least 2.1, at least 2.1, at least 2.3, at least 2.4, at least 2.5, at least 2.6, at least 2.7, at least 2.8, at least 2.9, at least 3) fold in NLRP3 in the sample obtained from the patient relative to the corresponding reference value, indicates that the patient has, or is at risk of developing SIRS. In one embodiment, no increase or an increase of less than 2 (eg. less than 2.1, less than 2.2, less than 2.3, less than 2.4, or less than 2.5) fold in NLRP3 in the sample obtained from the patient relative to the corresponding reference value, indicates that the patient does not have, or is not at risk of developing SIRS.

For example, an increase of at least 3.5 (eg. at least 3.6, at least 3.7, at least 3.8, at least 3.9, or at least 4) fold in RBP4 in the sample obtained from the patient relative to the corresponding reference value, indicates that the patient has, or is at risk of developing SIRS. In one embodiment, no increase or an increase of less than 3.5 (eg. less than 3.6, less than 3.7, less than 3.8, less than 3.9, or less than 4) fold in RBP4 in the sample obtained from the patient relative to the corresponding reference value, indicates that the patient does not have, or is not at risk of developing SIRS. In one embodiment, when detecting this biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 2 (eg. at least 2.1, at least 2.2, at least 2.3, at least 2.4, at least 2.5) fold in MPP3 in the sample obtained from the patient relative to the corresponding reference value, indicates that the patient has, or is at risk of developing SIRS. In one embodiment, no increase or an increase of less than 2 (eg. less than 2.1, less than 2.2, less than 2.3, less than 2.4, less than 2.5) fold in MPP3 in the sample obtained from the patient relative to the corresponding reference value, indicates that the patient does not have, or is not at risk of developing SIRS.

As described herein, the present inventors observed that the levels of the one or more SIRS biomarkers were elevated in patients having SIRS as compared to patients having sepsis. Detection of increased levels of these biomarkers in a patient as compared to the levels detected for patients having sepsis can thus be used to diagnose the presence of SIRS.

Thus, in one embodiment, the reference value used in the method of the invention is representative of an individual (or population of individuals) having sepsis (such as abdominal sepsis and/or pulmonary sepsis). The reference value that is representative of an individual having sepsis is as described above for the “method for diagnosing a systemic inflammatory condition in a patient”.

In one embodiment, an increase in the one or more biomarker (and/or one or more additional biomarker) in the sample obtained from the patient relative to the corresponding reference value representative of an individual having sepsis, indicates that the patient has or is at risk of developing SIRS. Likewise, no increase in the one or more biomarker (and/or one or more additional biomarker) in the sample obtained from the patient relative to the corresponding reference value representative of an individual having sepsis, indicates that the patient does not have SIRS.

In one embodiment, the patient may be diagnosed as having SIRS, or being at risk of developing SIRS, when the one or more biomarker (or the one or more additional biomarker) increases by at least 1 (e.g. at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value of an individual having sepsis.

The method for diagnosis of SIRS as described herein can be used in a decision tree process to investigate the health of a patient having or suspected of having a systemic inflammatory condition. For example, the method for diagnosis of SIRS in a patient can be performed before, after, or in addition to any of the other methods described herein.

In one embodiment, the method for diagnosing SIRS in a patient (as described herein) can be performed subsequent to (or in addition to) the method for diagnosing whether a patient has a systemic inflammatory condition (as described herein). If the patient tests positive for a systemic inflammatory condition (using the method of the invention for diagnosing whether a patient has a systemic inflammatory condition), they may be tested for SIRS using the diagnostic method described herein. Furthermore, the method for diagnosis of SIRS may be performed before, after, or in addition to the method for diagnosis of sepsis in a patient, as described herein.

In one embodiment, the method of the invention for diagnosing SIRS in a patient (as described herein) can be performed subsequent to (or in addition to) the method for distinguishing between sepsis and SIRS in a patient (as described herein). If the patient tests positive for SIRS using the distinguishing method of the invention, they may then be tested for SIRS using the diagnostic method described herein, so as to further confirm the diagnosis of SIRS in the patient.

In one embodiment, the method for diagnosis of SIRS may be performed subsequent to (or in addition to) the method for diagnosing whether a patient has a systemic inflammatory condition (as described herein), and the method for distinguishing between sepsis and SIRS in a patient (as described herein). For example, the patient may be tested first using the method for diagnosing whether a patient has a systemic inflammatory condition (as described herein). If the patient tests positive for a systemic inflammatory condition, they may be tested using the distinguishing method of the invention (as described herein) to determine whether they have sepsis and/or SIRS. If the patient tests positive for SIRS using the distinguishing method of the invention, they may be tested for SIRS using the diagnostic method described herein, so as to further confirm the diagnosis of SIRS in the patient. Furthermore, the method for diagnosis of SIRS may be performed before, after, or in addition to the method for diagnosis of sepsis in a patient, as described herein.

The methods for diagnosis of a systemic inflammatory condition, sepsis, abdominal sepsis, pulmonary sepsis and/or SIRS may be performed simultaneously or sequentially in any order. The above described combination of methods may be performed in parallel to determine the disease status of a patient by simultaneously (or substantially simultaneously) investigating the expression of all the biomarkers in a sample obtained from the patient, and determining whether the patient has or is at risk of having a systemic inflammatory condition, sepsis (such as abdominal or pulmonary sepsis) and/or SIRS.

When performing these different methods in a decision tree process, the sample used in each step of the method may be the same sample obtained from the patient (as described herein). When the method comprises multiple quantification steps, all the steps may be performed at the same time (e.g. in parallel) and/or using the same sample.

In a related aspect, the present invention also provides the use of one or more of: PLA2G7, ARHGEF10L, MYCL, TGFBI, GPR124, URN, NLRP3, RBP4, and MPP3, as a biomarker for SIRS. In one embodiment, the use is of the one or more biomarker in the diagnosis of SIRS in a patient. In one embodiment, the use is of one or more of: PLA2G7, ARHGEF10L, MYCL, TGFBI, and GPR124. In one embodiment, the use is of one or more of: PLA2G7, ARHGEF10L, MYCL, and TGFBI. For example, the use may be of the combination of: PLA2G7, ARHGEF10L, MYCL, and TGFBI.

For example, the use may comprise (i) determining the presence and/or amount of one or more biomarker in a sample obtained from a patient; and (ii) comparing the presence and/or amount of the one or more biomarker determined in said sample in (i) to a corresponding reference value to determine whether the patient has or is at risk of developing SIRS.

All embodiments described above for the method of diagnosing SIRS in a patient apply equally to the ‘use’ of the invention described herein. This includes all embodiments relating to the terms “SIRS”, “patient”, “sample”, and “the one or more biomarker”.

### Diagnosis of Sepsis

When investigating gene expression patterns in patients having systemic inflammatory conditions, the inventors identified a subset of biomarkers (see Table 3) that were expressed at different levels in patients having sepsis, as compared to patients having other systemic inflammatory conditions, and healthy individuals. As a result of these findings, the inventors thus observed that ITGB3, ITGA2B, MYL9, LCN2, TREML1, LCN15, CMTM5, PPBP, PF4, KIF2C, MAP1A, SELP, NEXN, NLRC4, CLEC1B, MRAS, PCOLCE2, TMEM37, SLC39A8, KIF2C, CIQC, CIQB, CIQA, TNF, IF144, IFIT1, RPGRIP1, EPSTI1, DISC1, CXCR1, and HCAR2, can be used as biomarkers for diagnosis of sepsis.

In particular, the present inventors have identified that ITGB3, ITGA2B, MYL9, LCN2, TREML1, LCN15, CMTM5, PPBP, PF4, KIF2C, MAP1A, SELP, NEXN, NLRC4, and CLEC1B, are elevated in all types of sepsis tested, and thus can be used as biomarkers for sepsis including abdominal sepsis and pulmonary sepsis.

The inventors also observed that the levels of SLC39A8, CIQC, CIQA, MRAS, TMEM37, CIQB, PCOLCE2, KIF2C, and TNF are elevated in patients having abdominal sepsis, compared to patients having pulmonary sepsis or SIRS, and healthy individuals. The inventors also observed that the levels of IF144, IFIT1, and RPGRIP1 were decreased in patients having abdominal sepsis, compared to patients having pulmonary sepsis or SIRS, and healthy individuals. SLC39A8, CIQC, CIQA, MRAS, TMEM37, CIQB, PCOLCE2, KIF2C, TNF, IF144, IFIT1, and RPGRIP1 can thus be used as biomarkers for abdominal sepsis. Likewise, the inventors observed that the levels of HCAR2, CXCR1, DISC1, EPSTI1, and IF144 are elevated in patients having pulmonary sepsis, compared to patients having abdominal sepsis and/or SIRS, and healthy individuals. HCAR2, CXCR1, DISC1, EPSTI1, and IF144 can thus be used as biomarkers for pulmonary sepsis.

The present invention therefore provides a method for diagnosing sepsis in a patient, comprising:

(i) determining the presence and/or amount of one or more biomarker in a sample obtained from a patient, wherein the one or more biomarker is selected from the group consisting of: ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC, CIQA, MRAS, TMEM37, CIQB, PCOLCE2, KIF2C, TNF, IF144, IFIT1, RPGRIP1, HCAR2, CXCR1, DISC1, and EPSTI1;
(ii) comparing the presence and/or amount of the one or more biomarker determined in said sample in (i) to a corresponding reference value; and thereby determining whether the patient has sepsis.

All embodiments described above for the “method for diagnosing a systemic inflammatory condition in a patient” apply equally to the “method for diagnosing sepsis in a patient”. This includes all embodiments relating to the “sample”, “patient”, “biomarker”, and “reference value”, and all embodiments relating to the step for “determining the presence and/or amount of one or more biomarker in a sample” and for the “comparison” step for making a conclusion about the disease status of the patient.

As used herein, the phrase “diagnosis of sepsis in a patient” means determining whether the patient has or is risk of developing sepsis. The systemic inflammatory condition “sepsis” diagnosed using the method of the invention is as described above for the “method for diagnosing a systemic inflammatory condition in a patient”. In one embodiment, the method is for diagnosing one or more of: abdominal sepsis and pulmonary sepsis. In one embodiment, the method is for diagnosing abdominal sepsis. In one embodiment, the method is for diagnosing pulmonary sepsis.

The “patient” for which diagnosis is performed is as described above for the “method for diagnosing a systemic inflammatory condition in a patient”. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having a systemic inflammatory condition using the method described herein. In one embodiment, the patient is suspected of having or being at risk of developing sepsis. In one embodiment, the patient has been diagnosed as having or being at risk of developing sepsis using the method described herein for distinguishing between sepsis and SIRS in a patient.

The “sample” obtained from the patient is as described above for the “method for diagnosing a systemic inflammatory condition in a patient”, including all embodiments relating to the time point at which the sample is obtained.

The optimum time point at which a sample is obtained from a patient may depend on the biomarker being tested. For example, when testing for any one or more of the biomarkers MAP1A, SELP, NEXN, ITGA2B, MYL9, CMTM5, PPBP, TREML1, PF4, CLEC1B or ITGB3, the sample may be obtained up to 1 hour, 2 hours, 4 hours, 6 hours, 8 hours, 12 hours, 24 hours, 36 hours, 48 hours, 72 hours, or 96 hours after the patient presents with one or more clinical symptoms of a systemic inflammatory condition, or is admitted to a medical care facility. Preferably, the sample is obtained up to 24 hours after the patient presents with one or more clinical symptoms of a systemic inflammatory condition, or is admitted to a medical care facility. Preferably, the sample is obtained up to 48 hours after the patient presents with one or more clinical symptoms of a systemic inflammatory condition, or is admitted to a medical care facility.

The “one or more biomarker” of the invention is as described above for the “method for diagnosing a systemic inflammatory condition in a patient”.

As illustrated in Example 1, the present inventors observed that ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC, CIQA, MRAS, TMEM37, CIQB, PCOLCE2, KIF2C, TNF, IFI44, IFIT1, RPGRIP1, HCAR2, CXCR1, DISC1, and EPSTI1, are biomarkers of sepsis, and thus can be used in the diagnosis of sepsis.

The reference to the biomarker SLC39A8 throughout the entire description, includes the transcript variant 1 of SLC39A8 (as encoded by SEQ ID NO: 70) and the transcript variant 3 of SLC39A8 (as encoded by SEQ ID NO: 71). In one embodiment, the reference to the biomarker SLC39A8 is a reference to the transcript variant 1 of SLC39A8 (as encoded by SEQ ID NO: 70). In one embodiment, the reference to the biomarker SLC39A8 is a reference to the transcript variant 3 of SLC39A8 (as encoded by SEQ ID NO: 71).

In one embodiment, the one or more biomarker may be selected from the group consisting of ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC CIQA, MRAS, TMEM37, CIQB, PCOLCE2, KIF2C, TNF, IFI44, IFIT1, RPGRIP1, HCAR2, CXCR1, DISC1, and EPSTI1.

In one embodiment, the one or more biomarker may be selected from the group consisting of ITGB3, ITGA2B, MYL9, LCN2, TREML1, LCN15, CMTM5, PPBP, PF4, MAP1A, SELP, NLRC4, IFI44, HCAR2, CXCR1, DISC1, and EPSTI1. When detecting one or more biomarker selected from this sub-group, the systemic inflammatory condition diagnosed using the method may be pulmonary sepsis.

The present inventors observed that a sub-set of the biomarkers for sepsis specifically increased in abundance in all types of sepsis tested (including abdominal and pulmonary sepsis) as compared to healthy individuals and patients having SIRS. These markers are therefore useful for diagnosis of sepsis in a patient. In one embodiment, the one or more biomarker may be selected from the group consisting of: ITGB3, ITGA2B, MYL9, LCN2, TREML1, LCN15, CMTM5, PPBP, PF4, KIF2C, MAP1A, SELP, NEXN, NLRC4, and CLEC1B.

The present inventors also observed that a sub-set of the biomarkers for sepsis (ITGB3, ITGA2B, MYL9, LCN2, TREML1, LCN15, CMTM5, PPBP, and PF4) increase in abundance in patients having sepsis as compared to healthy individuals, and show no increase (or a decrease) in patients having SIRS as compared to healthy individuals (eg. in patients tested at days 1 and 2 post-hospitalisation). These markers therefore provide highly specific biomarkers for diagnosing sepsis. Thus, in one embodiment, the one or more biomarker may therefore be selected from the group consisting of: ITGB3, ITGA2B, MYL9, LCN2, TREML1, LCN15, CMTM5, PPBP, and PF4.

Furthermore, of this sub-set of sepsis biomarkers, the inventors also observed that the specific biomarkers ITGB3, ITGA2B, MYL9, LCN2, and TREML1 were particularly effective at diagnosing sepsis when tested using ROC analysis, as described in Example 2. Specifically, AUC values of 0.86, 0.83, 0.82, 0.82 and 0.8 were observed for ITGB3, ITGA2B, MYL9, LCN2, and TREML1. Thus, in one embodiment, the one or more biomarker is preferably selected from the group consisting of: ITGB3, ITGA2B, MYL9, LCN2, and TREML1.

Each of the biomarkers of sepsis may be used alone, or in combination with any of the sepsis biomarkers in the method of the invention. For example, any combination of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, up to and including all of the sepsis biomarkers may be used to diagnose sepsis in a patient according to the method of the invention.

In one embodiment, the one or more biomarker is LCN2. In one embodiment, the one or more biomarker is ITGA2B. In one embodiment, the one or more biomarker is MYL9. In one embodiment, the one or more biomarker is ITGB3. In one embodiment, the one or more biomarker is TREML1. In one embodiment, the one or more biomarker is LCN15. In one embodiment, the one or more biomarker is CMTM5. In one embodiment, the one or more biomarker is PPBP. In one embodiment, the one or more biomarker is PF4. In one embodiment, the one or more biomarker is KIF2C. In one embodiment, the one or more biomarker is MAP1A. In one embodiment, the one or more biomarker is SELP. In one embodiment, the one or more biomarker is NEXN. In one embodiment, the one or more biomarker is NLRC4. In one embodiment, the one or more biomarker is CLEC1B. In one embodiment, the one or more biomarker is MRAS. In one embodiment, the one or more biomarker is CIQC. In one embodiment, the one or more biomarker is CIQB. In one embodiment, the one or more biomarker is PCOLCE2. In one embodiment, the one or more biomarker is CIQA. In one embodiment, the one or more biomarker is TMEM37. In one embodiment, the one or more biomarker is SLC39A8. In one embodiment, the one or more biomarker is TNF. In one embodiment, the one or more biomarker is IF144. In one embodiment, the one or more biomarker is IFIT1. In one embodiment, the one or more biomarker is RPGRIP1. In one embodiment, the one or more biomarker is EPSTI1. In one embodiment, the one or more biomarker is DISC1. In one embodiment, the one or more biomarker is CXCR1. In one embodiment, the one or more biomarker is HCAR2.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, or all 29) of the biomarkers selected from the group consisting of: ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC, CIQA, MRAS, TMEM37, CIQB, PCOLCE2, KIF2C, TNF, IF144, IFIT1, RPGRIP1, HCAR2, CXCR1, DISC1, and EPSTI1, may be used to diagnose sepsis in a patient.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, or all 15) of the biomarkers selected from the group consisting of: ITGB3, ITGA2B, MYL9, LCN2, TREML1, LCN15, CMTM5, PPBP, PF4, KIF2C, MAP1A, SELP, NEXN, NLRC4, and CLEC1B, may be used to diagnose sepsis in a patient.

In one embodiment, any combination of 1 or more (eg. 2 or more, 3 or more, 4 or more, 5 or more, 6 or more, 7 or more, 8 or more, or all 9) of the biomarkers selected from the group consisting of: ITGB3, ITGA2B, MYL9, LCN2, TREML1, LCN15, CMTM5, PPBP, and PF4, may be used to diagnose sepsis in a patient.

In one embodiment, any combination of 1 or more (eg. 2 or more, 3 or more, 4 or more, or all 5) of the biomarkers selected from the group consisting of: ITGB3, ITGA2B, MYL9, LCN2, and TREML1, may be used to diagnose sepsis in a patient. For example, the method may be performed using 2 or more of the biomarkers selected from the group consisting of: ITGB3, ITGA2B, MYL9, LCN2, and TREML1. For example, the method may be performed using 3 or more of the biomarkers selected from the group consisting of: ITGB3, ITGA2B, MYL9, LCN2, and TREML1. For example, the method may be performed using 4 or more of the biomarkers selected from the group consisting of: ITGB3, ITGA2B, MYL9, LCN2, and TREML1. For example, the method may be performed using all 5 biomarkers: ITGB3, ITGA2B, MYL9, LCN2, and TREML1. This combination of sepsis biomarkers was shown to be particularly effective in diagnosing sepsis when tested by ROC analysis described in Example 2.

For example, the following combinations of sepsis biomarkers may be used in the method of the invention to diagnose sepsis: (i) LCN15 and ITGA2B; (ii) LCN15 and MYL9; (iii) LCN15 and CMTM5; (iv) LCN15 and PPBP; (v) LCN15 and TREML1; (vi) LCN15 and PF4; (vii) LCN15 and LCN2; (viii) LCN15 and ITGB3; (ix) ITGA2B and MYL9; (x) ITGA2B and CMTM5; (xi) ITGA2B and PPBP; (xii) ITGA2B and TREML1; (xiii) ITGA2B and PF4; (xiv) ITGA2B and LCN2; (xv) ITGA2B and ITGB3; (xvi) MYL9 and CMTM5; (xvii) MYL9 and PPBP; (xviii) MYL9 and TREML1; (xix) MYL9 and PF4; (xx) MYL9 and LCN2; (xxi) MYL9 and ITGB3; (xxii) CMTM5 and PPBP; (xxiii) CMTM5 and TREML1; (xxiv) CMTM5 and PF4; (xxv) CMTM5 and LCN2; (xxvi) CMTM5 and ITGB3; (xxvii) PPBP and TREML1; (xxviii) PPBP and PF4; (xxix) PPBP and LCN2; (xxx) PPBP and ITGB3; (xxxi) TREML1 and PF4; (xxxii) TREML1 and LCN2; (xxxiii) TREML1 and ITGB3; (xxxiv) PF4 and LCN2; (xxxv) PF4 and ITGB3; and (xxxvi) LCN2 and ITGB3.

One or more additional biomarker for sepsis may also be used in the diagnosis of sepsis according to the method of the invention. Any combination of the one or more additional biomarker may be used in combination with the one or more biomarker of the invention. For example at least 1, at least 2, at least 3, at least 4, at least 5, at least 6, at least 7, at least 8, at least 9, at least 10, at least 11, at least 12, at least 13, or all 14 additional biomarkers for sepsis may be used in combination with the one or more biomarker of the invention (as described herein). Typically, the one or more additional biomarker is selected from the group consisting of: ITGB3, ITGA2B, MYL9, LCN2, TREML1, LCN15, CMTM5, PPBP, PF4, KIF2C, MAP1A, SELP, NEXN, NLRC4, and CLEC1B. For example, the one or more additional biomarker may be selected from the group consisting of: ITGB3, ITGA2B, MYL9, LCN2, TREML1, LCN15, CMTM5, PPBP, and PF4. For example, the one or more additional biomarker may be selected from the group consisting of: ITGB3, ITGA2B, MYL9, LCN2, and TREML1.

In one embodiment, the one or more biomarker is LCN2, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, TREML1, LCN15, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, TREML1, LCN15, CMTM5, PPBP, and PF4. In one embodiment, the one or more additional biomarker is selected from at least 1 (eg. at least 2, at least 3, up to and including all) of the biomarkers: ITGB3, ITGA2B, MYL9, and TREML1.

In one embodiment, the one or more biomarker is ITGA2B, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, MYL9, LCN2, TREML1, LCN15, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, MYL9, LCN2, TREML1, LCN15, MYL9, ITGB3, CMTM5, PPBP, and PF4. In one embodiment, the one or more additional biomarker is selected from at least 1 (eg. at least 2, at least 3, up to and including all) of the biomarkers: ITGB3, MYL9, LCN2, and TREML1.

In one embodiment, the one or more biomarker is MYL9, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, LCN2, TREML1, LCN15, ITGA2B, ITGB3, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, LCN2, TREML1, LCN15, ITGA2B, ITGB3, CMTM5, PPBP, and PF4. In one embodiment, the one or more additional biomarker is selected from at least 1 (eg. at least 2, at least 3, up to and including all) of the biomarkers: ITGB3, ITGA2B, LCN2, and TREML1.

In one embodiment, the one or more biomarker is ITGB3, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGA2B, MYL9, LCN2, TREML1, LCN15, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGA2B, MYL9, LCN2, TREML1, LCN15, ITGA2B, MYL9, CMTM5, PPBP, and PF4. In one embodiment, the one or more additional biomarker is selected from at least 1 (eg. at least 2, at least 3, up to and including all) of the biomarkers: ITGA2B, MYL9, LCN2, and TREML1.

In one embodiment, the one or more biomarker is TREML1, and the one or more additional biomarker is selected from at least 1 (eg. at least 2, at least 3, at least 4, at least 5, at least 6, at least 7, at least 8, at least 9, at least 10, at least 11, up to and including all) of the biomarkers: ITGB3, ITGA2B, MYL9, LCN2, LCN15, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, LCN15, CMTM5, PPBP, and PF4. In one embodiment, the one or more additional biomarker is selected from at least 1 (eg. at least 2, at least 3, up to and including all) of the biomarkers: ITGB3, ITGA2B, MYL9, and LCN2.

In one embodiment, the one or more biomarker is LCN15, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2 TREML1, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, TREML1, CMTM5, PPBP, and PF4.

In one embodiment, the one or more biomarker is CMTM5, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, TREML1, LCN15, PPBP, and PF4.

In one embodiment, the one or more biomarker is PPBP, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, TREML1, LCN15, ITGA2B, MYL9, ITGB3, CMTM5, and PF4.

In one embodiment, the one or more biomarker is PF4, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PPBP, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, TREML1, LCN15, ITGA2B, MYL9, ITGB3, CMTM5, and PPBP.

In one embodiment, the one or more biomarker is KIF2C, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, LCN15, TREML1, ITGA2B, MYL9, ITGB3, CMTM5, PPBP, PF4, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, TREML1, LCN15, ITGA2B, MYL9, ITGB3, CMTM5, PPBP, and PF4.

In one embodiment, the one or more biomarker is MAP1A, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PPBP, PF4, KIF2C, SELP, NEXN, NLRC4, and CLEC1B. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, TREML1, LCN15, ITGA2B, MYL9, ITGB3, CMTM5, PPBP, and PF4.

In one embodiment, the one or more biomarker is SELP, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PPBP, PF4, KIF2C, MAP1A, NEXN, NLRC4, and CLEC1B. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, LCN15, TREML1, ITGA2B, MYL9, ITGB3, CMTM5, PPBP, and PF4.

In one embodiment, the one or more biomarker is NEXN, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PPBP, PF4, KIF2C, MAP1A, SELP, NLRC4, and CLEC1B. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, TREML1, LCN15, ITGA2B, MYL9, ITGB3, CMTM5, PPBP, and PF4.

In one embodiment, the one or more biomarker is NLRC4, and the one or more additional biomarker is selected from at least 1 (eg. at least 2, at least 3, at least 4, at least 5, at least 6, at least 7, at least 8, at least 9, at least 10, at least 11, up to and including all) of the biomarkers: ITGB3, ITGA2B, MYL9, LCN2, TREML1, LCN15, ITGA2B, MYL9, ITGB3, CMTM5, PPBP, PF4, KIF2C, MAP1A, SELP, NEXN, and CLEC1B. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, TREML1, LCN15, ITGA2B, MYL9, ITGB3, CMTM5, PPBP, and PF4.

In one embodiment, the one or more biomarker is CLEC1B, and the one or more additional biomarker is selected from at least 1 (eg. at least 2, at least 3, at least 4, at least 5, at least 6, at least 7, at least 8, at least 9, at least 10, at least 11, at least 12, at least 13, up to and including all) of the biomarkers: ITGB3, ITGA2B, MYL9, LCN2, TREML1, LCN15, ITGA2B, MYL9, ITGB3, CMTM5, PPBP, PF4, KIF2C, MAP1A, SELP, NEXN, and NLRC4. In one embodiment, the one or more additional biomarker is selected from at least 1 (eg. at least 2, at least 3, at least 4, at least 5, at least 6, at least 7, at least 8, up to and including all) of the biomarkers: ITGB3, ITGA2B, MYL9, LCN2, TREML1, LCN15, ITGA2B, MYL9, ITGB3, CMTM5, PPBP, and PF4.

As illustrated in FIG. 3, the present inventors observed that the “sepsis” biomarkers described herein increase in abundance in patients having sepsis as compared to patients having other systemic inflammatory conditions (such as SIRS), and as compared to healthy individuals. These differences in marker abundance can be used to diagnose whether an individual has or is at risk of developing sepsis.

For example, by comparing the presence and/or amount of markers quantified in a sample obtained from a patient to the presence and/or amount of markers quantified for a reference value (such as a reference value that is representative of a healthy individual (or a population of healthy individuals), or a reference value that is representative of an individual (or a population of individuals) having sepsis eg. abdominal and/or pulmonary sepsis, or a reference value that is representative of an individual (or a population of individuals) having SIRS, it is possible to diagnose the presence (or absence) of sepsis in a patient. The method permits classification of the individual as belonging to or not belonging to the reference population (ie. by determining whether the amounts of marker quantified in the individual are statistically similar to the reference population or statistically deviate from the reference population). Hence, classification of the individual's marker profile (ie. the overall pattern of change observed for the markers quantified) as corresponding to the profile derived from a particular reference population is predictive that the individual falls (or does not fall) within the reference population.

In one embodiment, an individual may be diagnosed as having or being at risk of having sepsis, when the amount of the one or more biomarker is statistically similar to the amount determined for the corresponding reference value representative of an individual (or a population of individuals) having sepsis. In one embodiment, an individual may be diagnosed as not having or not being at risk of having sepsis when the amount of the one or more biomarker is statistically similar to the amount determined for the corresponding reference value representative of a healthy individual (or a population of healthy individuals). In one embodiment, an individual may be diagnosed as not having or not being at risk of having sepsis when the amount of the one or more biomarker is statistically similar to the amount determined for the corresponding reference value representative of an individual having SIRS (or a population of individuals having SIRS).

In one embodiment, an individual may be diagnosed as not having or not being at risk of having sepsis when the amount of the one or more biomarker statistically deviates from the amount determined for the corresponding reference values representative of an individual (or a population of individuals) having sepsis. In one embodiment, an individual may be diagnosed as having or being at risk of having sepsis when the amount of the one or more biomarker statistically deviates from the amount determined for the corresponding reference value representative of a healthy individual (or a population of healthy individuals). In one embodiment, an individual may be diagnosed as having or being at risk of having sepsis when the amount of the one or more biomarker statistically deviates from the amount determined for the corresponding reference values representative of an individual having SIRS (or a population of individuals having SIRS).

All embodiments described above for the classification of a patient as having or being at risk of having a systemic inflammatory condition (or as not having or not being at risk of having a systemic inflammatory condition) apply equally to the method for diagnosing whether a patient has or is at risk of having sepsis. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the method of diagnosing a systemic inflammatory condition in a patient. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having sepsis (or a population of individuals having sepsis). As described above for the “method for diagnosing a systemic inflammatory condition in a patient”, the method of the invention may involve the use of multiple separate reference values. For example, the reference value may include one of more (eg. two or more, or all 3) of the reference values selected from: a reference value that is representative of a healthy individual (or a population of healthy individuals); a reference value that is representative of an individual having SIRS (or a population of individuals having SIRS); and a reference value that is representative of an individual having sepsis (or a population of individuals having sepsis). The reference value that is representative of an individual having sepsis (or a population of individuals having sepsis) may be representative of an individual (or a population of individuals) having abdominal sepsis and/or pulmonary sepsis.

The present inventors observed that the sepsis biomarkers described herein each increase in abundance in samples obtained from patients having sepsis, as compared to healthy individuals. Detection of increased levels of these biomarkers in a patient as compared to the levels detected for healthy individuals can thus be used to diagnose the presence of sepsis. In one embodiment, when the reference value is representative of a healthy individual (or population of healthy individuals), an increase in the one or more biomarker for sepsis in the sample obtained from the patient relative to the corresponding reference value indicates that the patient has sepsis, or is at risk of developing sepsis. Likewise, no increase in the one or more biomarker for sepsis in the sample obtained from the patient relative to the corresponding reference value indicates that the patient does not have sepsis.

For some of the sepsis biomarkers identified by the present inventors, increased levels of these markers were also observed in patients having SIRS as compared to healthy individuals, although much bigger increases were observed for these biomarkers in the patients having sepsis. The accuracy of sepsis diagnosis can thus be improved by looking for a “minimum” fold change or % change in the levels of the one or more biomarkers as compared to the corresponding reference value that is representative of a healthy individual. The fold change or % change may be as defined above for the method for diagnosis of a systemic inflammatory condition.

In one embodiment, the patient is diagnosed as having sepsis, or being at risk of developing sepsis, when the one or more biomarker for sepsis (or the one or more additional biomarker) increases by at least 0.1 (e.g. at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, or at least 10, at least 15 fold, at least 20 fold, at least 30 fold, at least 40 fold, at least 50) fold in the sample obtained from the patient relative to the corresponding reference value representative of a healthy individual.

For example, an increase of at least 1 (eg. at least 1.1, at least 1.2, at least 1.3, at least 1.4, or at least 1.5) fold in LCN15 in the sample obtained from the patient relative to the corresponding reference value, indicates that the patient has, or is at risk of developing sepsis. In one embodiment, no increase or an increase of less than 1 (eg. less than 1.1, less than 1.2, less than 1.3, less than 1.4, less than 1.5) fold in LCN15 in the sample obtained from the patient relative to the corresponding reference value, indicates that the patient does not have, or is not at risk of developing sepsis.

For example, an increase of at least 2 (eg. at least 2.1, at least 2.2, at least 2.3, at least 2.4, at least 2.5) fold in LCN2 in the sample obtained from the patient relative to the corresponding reference value, indicates that the patient has, or is at risk of developing sepsis. In one embodiment, no increase or an increase of less than 2.1 (eg. less than 2.2, less than 2.3) fold in LCN2 in the sample obtained from the patient relative to the corresponding reference value, indicates that the patient does not have, or is not at risk of developing sepsis.

For example, an increase of at least 1.5 (eg. at least 1.6, at least 1.7, at least 1.8, at least 1.9, at least 2, at least 2.5, at least 3, or at least 3.5) fold in ITGA2B in the sample obtained from the patient relative to the corresponding reference value, indicates that the patient has, or is at risk of developing sepsis. In one embodiment, no increase or an increase of less than 1.1 (eg. less than 1.2, less than 1.3, less than 1.4, less than 1.5, less than 1.6, less than 1.7, less than 1.8, less than 1.9, less than 2, less than 2.5, or less than 3) fold in ITGA2B in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1 (eg. at least 1.1, at least 1.2, at least 1.3, at least 1.4, at least 1.5, at least 1.6, at least 1.7, at least 1.8, at least 1.9, at least 2, at least 2.1, at least 2.2, at least 2.3, at least 2.4, or at least 2.5) fold in MYL9 in the sample obtained from the patient relative to the corresponding reference value, indicates that the patient has, or is at risk of developing sepsis. In one embodiment, no increase or an increase of less than 1 (eg. less than 1.1, less than 1.2, less than 1.3, less than 1.4, less than 1.5, less than 2) fold in MYL9 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 2 (eg. at least 2.1, at least 2.2, at least 2.3, at least 2.4, at least 2.5) fold in ITGB3 in the sample obtained from the patient relative to the corresponding reference value, indicates that the patient has, or is at risk of developing sepsis. In one embodiment, no increase or an increase of less than 2.1 (eg. less than 2, less than 1.9, less than 1.8, less than 1.7, less than 1.6, less than 1.5, less than 1.4, less than 1.3, less than 1.2) fold in ITGB3 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.1 (eg. at least 1.2 at least 1.3, at least 1.4, at least 1.5, at least 2, at least 2.1, at least 2.2, at least 2.3, at least 2.4, or at least 2.5) fold in CMTM5 in the sample obtained from the patient relative to the corresponding reference value, indicates that the patient has, or is at risk of developing sepsis. In one embodiment, no increase or an increase of less than 1.1 (eg. less than 1.2, less than 1.3, less than 1.4, less than 1.5) fold in CMTM5 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1 (eg. at least 1.1, at least 1.2, at least 1.3, at least 1.4, at least 1.5, at least 2 or at least 2.5) fold in PPBP in the sample obtained from the patient relative to the corresponding reference value, indicates that the patient has, or is at risk of developing sepsis. In one embodiment, no increase or an increase of less than 1 (eg. less than 1.1, less than 1.2, less than 1.3) fold in PPBP in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1 (eg. at least 1.1, at least 1.2, at least 1.3, at least 1.4, at least 1.5, at least 2, at least 2.1, at least 2.2, at least 2.3, at least 2.4, or at least 2.5) fold in TREML1 in the sample obtained from the patient relative to the corresponding reference value, indicates that the patient has, or is at risk of developing sepsis. In one embodiment, no increase or an increase of less than 1 (eg. less than 1.1, less than 1.2, less than 1.3, less than 1.4, less than 1.5, less than 2) fold in TREML1 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1 (eg. at least 1.1, at least 1.2, at least 1.3, at least 1.4, at least 1.5, at least 1.6, at least 1.7, at least 1.8, at least 1.9, or at least 2) fold in PF4 in the sample obtained from the patient relative to the corresponding reference value, indicates that the patient has, or is at risk of developing sepsis. In one embodiment, no increase or an increase of less than 1 (eg. less than 1.1, less than 1.2, less than 1.3, less than 1.4, less than 1.5, less than 1.6, less than 1.7, or less than 1.8) fold in PF4 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.5 (eg. at least 1.6, at least 1.7, at least 1.8, at least 1.9, at least 2, at least 2.1, at least 2.2, at least 2.3, at least 2.4, at least 2.5) fold in KIF2C in the sample obtained from the patient relative to the corresponding reference value, indicates that the patient has, or is at risk of developing sepsis (such as abdominal sepsis). In one embodiment, no increase or an increase of less than 1.5 (eg. less than 1.6, less than 1.7, less than 1.8, less than 1.9, less than 2) fold in KIF2C in the sample obtained from the patient relative to the corresponding reference value, indicates that the patient does not have, or is not at risk of developing sepsis (such as abdominal sepsis). In one embodiment, when detecting this level of fold change in the biomarker, the method is performed using a sample obtained from a patient at least 36 (eg. at least 48) hours after the patient presents with one or more clinical symptoms of a systemic inflammatory condition, or is admitted to a medical care facility. In one embodiment, when detecting this level of fold change in the biomarker, the method is performed using a sample obtained from a patient between about 24 and 48 hours after the patient presents with one or more clinical symptoms of a systemic inflammatory condition, or is admitted to a medical care facility. In one embodiment, when detecting this level of fold change in the biomarker, the method is for diagnosing abdominal sepsis in a patient.

For example, an increase of at least 1.2 (eg. at least 1.3, at least 1.4, at least 1.5, at least 1.6, at least 1.7, at least 1.8, at least 1.9, at least 2, at least 2.1, at least 2.2, at least 2.3, at least 2.4, or at least 2.5) fold in MAP1A in the sample obtained from the patient relative to the corresponding reference value, indicates that the patient has, or is at risk of developing sepsis. In one embodiment, no increase or an increase of less than 1.2 (eg. less than 1.3, less than 1.4, less than 1.5, less than 1.6, less than 1.7, less than 1.8) fold in MAP1A in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.5 (eg. at least 2, at least 2.1, at least 2.2, at least 2.3, at least 2.4, at least 2.5, at least 2.6, at least 2.7, at least 2.8, at least 2.9, or at least 3) fold in SELP in the sample obtained from the patient relative to the corresponding reference value, indicates that the patient has, or is at risk of developing sepsis. In one embodiment, no increase or an increase of less than 1.2 (eg. less than 1.3, less than 1.4, less than 1.5, less than 2) fold in SELP in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.8 (eg. at least 1.9, at least 2, at least 2.1, at least 2.2, at least 2.3, at least 2.4, or at least 2.5) fold in the amount of NEXN in the sample obtained from the patient relative to the corresponding reference value, indicates that the patient has, or is at risk of developing sepsis. In one embodiment, no increase or an increase of less than 1.1 (eg. less than 1.2, less than 1.3, less than 1.4, less than 1.5, less than 1.6, less than 1.7, less than 1.8, less than 1.9, less than 2) fold in NEXN in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 3.2 (eg. at least 3.3, at least 3.4, at least 3.5, at least 3.6, at least 3.7, at least 3.8, at least 3.9, at least 4) fold in NLRC4 in the sample obtained from the patient relative to the corresponding reference value, indicates that the patient has, or is at risk of developing sepsis. In one embodiment, no increase or an increase of less than 2.5 (eg. less than 2.6, less than 2.7, less than 2.8, less than 2.9, less than 3, less than 3.1, less than 3.2) fold in NLRC4 in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 2.7 (eg. at least 2.8, at least 2.9, at least 3, at least 3.1, at least 3.2, at least 3.3, at least 3.4, at least 3.5, at least 4) fold in CLEC1B in the sample obtained from the patient relative to the corresponding reference value, indicates that the patient has, or is at risk of developing sepsis. In one embodiment, no increase or an increase of less than 1.8 (eg. less than 1.9, less than 2, less than 2.1, less than 2.2, less than 2.3, less than 2.4, less than 2.5, less than 2.6, less than 2.7, less than 2.8, less than 2.9, less than 3) fold in CLEC1B in the sample obtained from the patient relative to the corresponding reference value, indicates that the patient does not have, or is not at risk of developing sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

As described herein, the present inventors observed that the levels of the one or more sepsis biomarkers were elevated in patients having sepsis as compared to patients having SIRS. Detection of increased levels of these biomarkers in a patient as compared to the levels detected for patients having SIRS can thus be used to diagnose the presence of sepsis. Thus, in one embodiment, when the reference value is representative of an individual having SIRS, an increase in the one or more biomarker (and/or one or more additional biomarker) in the sample obtained from the patient relative to the corresponding reference value representative of an individual having SIRS, indicates that the patient has or is at risk of developing sepsis. Likewise, no increase in the one or more biomarker (and/or one or more additional biomarker) in the sample obtained from the patient relative to the corresponding reference value representative of an individual having SIRS, indicates that the patient does not have or is not at risk of having sepsis. In one embodiment, the increase may be a minimum fold increase or a minimum % increase as defined above for the method for diagnosis of a systemic inflammatory condition.

In a related aspect, the present invention provides the use of one or more of: ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC, CIQA, MRAS, TMEM37, CIQB, PCOLCE2, KIF2C, TNF, IF144, IFIT1, RPGRIP1, HCAR2, CXCR1, DISC1, and EPSTI1, as a biomarker for sepsis. For example, the use is of one or more of: ITGB3, ITGA2B, MYL9, LCN2, TREML1, LCN15, CMTM5, PPBP, PF4, KIF2C, MAP1A, SELP, NEXN, NLRC4, and CLEC1B. For example, the use is of one or more of: ITGB3, ITGA2B, MYL9, LCN2, TREML1, LCN15, CMTM5, PPBP, and PF4. For example, the use is of one or more of: ITGB3, ITGA2B, MYL9, LCN2, and TREML1. For example, the use is of the combination of: ITGB3, ITGA2B, MYL9, LCN2, and TREML1. In one embodiment, the use is of the one or more biomarker in the diagnosis of sepsis in a patient. In one embodiment, the sepsis is abdominal sepsis and/or pulmonary sepsis. For example, the use may comprise (i) determining the presence and/or amount of one or more biomarker in a sample obtained from a patient; and (ii) comparing the presence and/or amount of the one or more biomarker determined in said sample in (i) to a corresponding reference value to determine whether the patient has sepsis.

All embodiments described above for the method of diagnosing sepsis in a patient apply equally to the ‘use’ of the invention described herein. This includes all embodiments relating to the terms “sepsis”, “patient”, “sample”, and “the one or more biomarker” described above.

As discussed herein, the present inventors observed that MRAS, PCOLCE2, TMEM37, SLC39A8, KIF2C, CIQC, CIQB, CIQA, TNF, IF144, IFIT1, and RPGRIP1, are biomarkers specific for abdominal sepsis (see Table 3).

The present invention thus also provides a method for diagnosing abdominal sepsis in a patient, comprising:

(i) determining the presence and/or amount of one or more biomarker in a sample obtained from a patient, wherein the one or more biomarker is selected from the group consisting of: SLC39A8, CIQC, CIQA, MRAS, TMEM37, CIQB, PCOLCE2, KIF2C, TNF, IF144, IFIT1, and RPGRIP1
(ii) comparing the presence and/or amount of the one or more biomarker determined in said sample in (i) to a corresponding reference value; and thereby determining whether the patient has or is at risk of developing abdominal sepsis.

All embodiments described above for the “method for diagnosing a systemic inflammatory condition in a patient” apply equally to the “method for diagnosing abdominal sepsis in a patient”. This includes all embodiments relating to the “sample”, “patient”, “biomarker”, and “reference value”, and all embodiments relating to the step for “determining the presence and/or amount of one or more biomarker in a sample” and for the “comparison” step for making a conclusion about the disease status of the patient.

As used herein, the phrase “diagnosis of abdominal sepsis in a patient” means determining whether the patient has or is risk of developing abdominal sepsis. The systemic inflammatory condition “abdominal sepsis” diagnosed using the method of the invention is as described above for the “method for diagnosing a systemic inflammatory condition in a patient”.

The “patient” for which diagnosis is performed is as described above for the “method for diagnosing a systemic inflammatory condition in a patient” and “the method for diagnosing sepsis in a patient”. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having a systemic inflammatory condition using the method described herein. In one embodiment, the patient is suspected of having or being at risk of developing sepsis. In one embodiment, the patient has been diagnosed as having or being at risk of developing sepsis (eg. using the method of diagnosing sepsis in a patient as described herein and/or using the method of distinguishing between sepsis and SIRS in a patient as described herein). In one embodiment, the patient is suspected of having or being at risk of developing abdominal sepsis.

The “sample” obtained from the patient is as defined above for the “method for diagnosing a systemic inflammatory condition in a patient”, including all embodiments relating to the time point at which the sample is obtained. All embodiments of the “sample” defined above for the method for diagnosing sepsis also apply to the method for diagnosing pulmonary sepsis.

The optimum time point at which a sample is obtained from a patient may depend on the biomarker being tested. For example, when testing for the biomarkers CIQC, CIQB, CIQA, and MRAS, the sample may be obtained up to 1 hour, 2 hours, 4 hours, 6 hours, 8 hours, 12 hours, 24 hours, 36 hours, 48 hours, 72 hours, or 96 hours after the patient presents with one or more clinical symptoms of a systemic inflammatory condition, or is admitted to a medical care facility. In one embodiment, the sample is obtained up to 24 hours after the patient presents with one or more clinical symptoms of a systemic inflammatory condition, or is admitted to a medical care facility. In one embodiment, the sample is obtained up to 48 hours after the patient presents with one or more clinical symptoms of a systemic inflammatory condition, or is admitted to a medical care facility.

The “one or more biomarker” of the invention is as defined above for the “method for diagnosing a systemic inflammatory condition in a patient”.

As illustrated in Example 1, the present inventors observed that SLC39A8, CIQC, CIQA, MRAS, TMEM37, CIQB, PCOLCE2 KIF2C, TNF, IF144, IFIT1, and RPGRIP1 are biomarkers of abdominal sepsis, and thus can be used in the diagnosis of abdominal sepsis.

In one embodiment, the one or more biomarker may be selected from the group consisting of: SLC39A8, CIQC, CIQA, MRAS, TMEM37, CIQB, PCOLCE2, KIF2C, TNF, IF144, IFIT1, and RPGRIP1.

A sub-set of the biomarkers identified (IF144, IFIT1, and RPGRIP1) were found at decreased levels in abdominal sepsis patients as compared to healthy individuals, individuals having SIRS, and/or individuals having pulmonary sepsis. In one embodiment, the one or more biomarker may be selected from the group consisting of: IF144, IFIT1, and RPGRIP1.

A sub-set of the biomarkers identified were found at elevated levels in abdominal sepsis patients as compared to healthy individuals, individuals having SIRS, and/or individuals having pulmonary sepsis. In one embodiment, the one or more biomarker may be selected from the group consisting of: SLC39A8, CIQC, CIQA, MRAS, TMEM37, CIQB, PCOLCE2, KIF2C, and TNF.

As described Example 2, a sub-set of the markers tested (SLC39A8, CIQC, CIQA, MRAS, TMEM37, and CIQB) was observed to provide particularly accurate diagnosis of abdominal sepsis (see the ROC curve data presented in Example 2). In one embodiment, the one or more biomarker may be selected from the group consisting of: SLC39A8, CIQC, CIQA, MRAS, TMEM37, and CIQB. In one embodiment, the one or more biomarker may be selected from the group consisting of: SLC39A8, CIQC, and CIQA.

The present inventors observed that the biomarker TNF is elevated in patients having abdominal sepsis as compared to healthy individuals and patients having pulmonary sepsis. However, the inventors observed that TNF is also elevated in patients having SIRS, and thus this marker is most useful in diagnosing abdominal sepsis when a patient has already been diagnosed as having sepsis (eg. using the methods described herein for diagnosis of sepsis, or using the method described herein for distinguishing between abdominal sepsis and pulmonary sepsis). Thus, in one embodiment, the one or more biomarker may be selected from the group consisting of: SLC39A8, CIQC, CIQA MRAS, TMEM37, CIQB, PCOLCE2, KIF2C, and optionally TNF. For example, the one or more biomarker may be selected from the group consisting of: SLC39A8, CIQC, CIQA MRAS, TMEM37, CIQB, PCOLCE2, and KIF2C. In one embodiment, the one or more biomarker may be selected from the group consisting of: SLC39A8, CIQC, CIQA, MRAS, TMEM37, CIQB, and optionally TNF.

In one embodiment, the one or more biomarker may be selected from the group consisting of: MRAS, CIQC, CIQB, and CIQA. In one embodiment, the one or more biomarker may be selected from the group consisting of: PCOLCE2, TMEM37, SLC39A8, KIF2C and TNF. In one embodiment, the one or more biomarker may be selected from the group consisting of: PCOLCE2, TMEM37, SLC39A8 and KIF2C.

Each of the biomarkers of abdominal sepsis may be used alone, or in combination with any of the abdominal sepsis biomarkers in the methods and uses of the invention. For example, any combination of 1 or more, 2 or more, 3 or more, 4 or more, 5 or more, 6 or more, 7 or more, 8 or more, 9 or more, 10 or more, 11 or more, or up to and including all of the abdominal sepsis biomarkers may be used to diagnose abdominal sepsis in a patient according to the methods and uses of the invention.

In one embodiment, any combination of 1 or more (eg. 2 or more, 3 or more, 4 or more, 5 or more, 6 or more, 7 or more, 8 or more, 9 or more, 10 or more, 11 or more, or all 12) of the biomarkers selected from the group consisting of: SLC39A8, CIQC, CIQA, MRAS, TMEM37, CIQB, PCOLCE2, KIF2C, TNF, IF144, IFIT1, and RPGRIP1 may be used to diagnose abdominal sepsis in a patient. In one embodiment, any combination of 1 or more (eg. 2 or more, 3 or more, 4 or more, 5 or more, 6 or more, 7 or more, 8 or more or all 9) of the biomarkers selected from the group consisting of: SLC39A8, CIQC, CIQA, MRAS, TMEM37, CIQB, PCOLCE2, KIF2C, and TNF may be used to diagnose abdominal sepsis in a patient. In one embodiment, any combination of 1 or more (eg. 2 or more, 3 or more, 4 or more, 5 or more, 6 or more, 7 or more or all 8) of the biomarkers selected from the group consisting of: SLC39A8, CIQC, CIQA, MRAS, TMEM37, CIQB, PCOLCE2, and KIF2C, may be used to diagnose abdominal sepsis in a patient. In one embodiment, any combination of 1 or more (eg. 2 or more, 3 or more, 4 or more or all 5) of the biomarkers selected from the group consisting of: MRAS, CIQC, CIQB, CIQA, and TNF may be used to diagnose abdominal sepsis in a patient. In one embodiment, when detecting one or more biomarker selected from this group, the sample is obtained from the patient up to 48 hours after the patient presents with one or more clinical symptoms of a systemic inflammatory condition, or is admitted to a medical care facility.

For example, the following combinations of abdominal sepsis biomarkers may be used to diagnose abdominal sepsis: (i) MRAS and CIQC; (ii) MRAS and CIQB; (iii) MRAS and PCOLCE2; (iv) MRAS and CIQA; (v) MRAS and TMEM37; (vi) MRAS and TNF; (vii) MRAS and SLC39A8; (viii) CIQC and CIQB; (ix) CIQC and PCOLCE2; (x) CIQC and CIQA; (xi) CIQC and TMEM37; (xii) CIQC and TNF; (xiii) CIQC and SLC39A8; (xiv) CIQB and PCOLCE2; (xv) CIQB and CIQA; (xvi) CIQB and TMEM37; (xvii) CIQB and TNF; (xviii) CIQB and SLC39A8; (xix) PCOLCE2 and CIQA; (xx) CIQA and TMEM37; (xxi) PCOLCE2 and TMEM37; (xxii) PCOLCE2 and TNF; (xxiii) PCOLCE2 and SLC39A8; (xxiv) CIQA and TNF; (xxv) CIQA and SLC39A8; (xxvi) TMEM37 and TNF; (xxvii) TMEM37 and SLC39A8; and (xxviii) TNF and SLC39A8; (xxxxix) MRAS and KIF2C; (xl) CIQC and KIF2C; (xli) CIQB and KIF2C; (xlii) CIQA and KIF2C; (xliii) TNF and KIF2C; (xliv) PCOLCE2 and KIF2C; (xlv) TMEM37 and KIF2C; (xlvi) SLC39A8 and KIF2C.

As described in Example 2, a sub-set of the biomarkers tested (SLC39A8, CIQC, CIQA, MRAS, TMEM37, and CIQB) was observed to provide particularly accurate diagnosis of abdominal sepsis (see the ROC curve data in Example 2). In one embodiment, any combination of 1 or more (eg. 2 or more, 3 or more, 4 or more, 5 or more, or all 6) of the biomarkers selected from the group consisting of: SLC39A8, CIQC, CIQA, MRAS, TMEM37, and CIQB, may be used to diagnose abdominal sepsis in a patient. For example, the combination of SLC39A8, CIQC, CIQA, MRAS, TMEM37, and CIQB may be used to diagnose abdominal sepsis in a patient. For example, 2 or more biomarkers selected from the group consisting of: SLC39A8, CIQC, CIQA, MRAS, TMEM37, and CIQB, may be used to diagnose abdominal sepsis in a patient. For example, 3 or more biomarkers selected from the group consisting of: SLC39A8, CIQC, CIQA, MRAS, TMEM37, and CIQB, may be used to diagnose abdominal sepsis in a patient. For example, 4 or more biomarkers selected from the group consisting of: SLC39A8, CIQC, CIQA, MRAS, TMEM37, and CIQB, may be used to diagnose abdominal sepsis in a patient. For example, the combination of the biomarkers: SLC39A8, CIQC, CIQA, MRAS, TMEM37, and CIQB, may be used to diagnose abdominal sepsis in a patient.

In one embodiment, any combination of 1 or more (eg. 2 or more, or all 3) of the biomarkers selected from the group consisting of: SLC39A8, CIQC, and CIQA may be used to diagnose abdominal sepsis in a patient. SLC39A8, CIQC, and CIQA may be used to diagnose abdominal sepsis in a patient. For example, 2 or more biomarkers selected from the group consisting of: SLC39A8, CIQC, and CIQA, may be used to diagnose abdominal sepsis in a patient. For example, the combination of the biomarkers: SLC39A8, CIQC, and CIQA may be used to diagnose abdominal sepsis in a patient

In one embodiment, the one or more biomarker is SLC39A8. In one embodiment, the one or more biomarker is CIQC. In one embodiment, the one or more biomarker is CIQA. In one embodiment, the one or more biomarker is CIQB. In one embodiment, the one or more biomarker is MRAS. In one embodiment, the one or more biomarker is TMEM37. In one embodiment, the one or more biomarker is PCOLCE2. In one embodiment, the one or more biomarker is KIF2C. In one embodiment, the one or more biomarker is TNF. In one embodiment, the one or more biomarker is IF144. In one embodiment, the one or more biomarker is IFIT1. In one embodiment, the one or more biomarker is RPGRIP1.

One or more additional biomarker for abdominal sepsis may also be used in the diagnosis of abdominal sepsis according to the method of the invention. Any combination of the one or more additional biomarker may be used in combination with the one or more biomarker of the invention. For example at least 1, at least 2, at least 3, at least 4, at least 5, at least 6, at least 7, at least 9, at least 10, or all 11 additional biomarkers for abdominal sepsis may be used in combination with the one or more biomarker of the invention (as described herein). Typically, the one or more additional biomarker is selected from the group consisting of: SLC39A8, CIQC, CIQA, MRAS, TMEM37, CIQB, PCOLCE2, KIF2C, TNF, IF144, IFIT1, and RPGRIP1. In one embodiment, the one or more additional biomarker is selected from the group consisting of: SLC39A8, CIQC, CIQA, MRAS, TMEM37, CIQB, PCOLCE2, KIF2C, and TNF. In one embodiment, the one or more additional biomarker is selected from the group consisting of: SLC39A8, CIQC, CIQA, MRAS, TMEM37, and CIQB.

In one embodiment, the one or more biomarker is MRAS, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, TMEM37, CIQB, PCOLCE2, KIF2C, TNF, IF144, IFIT1, and RPGRIP1. In one embodiment, the one or more additional biomarker is selected from the group consisting of: SLC39A8, CIQC, CIQA, TMEM37, and CIQB.

In one embodiment, the one or more biomarker is PCOLCE2, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, TMEM37, CIQB, KIF2C, TNF, IF144, IFIT1, and RPGRIP1.

In one embodiment, the one or more biomarker is TMEM37, and the one or more additional biomarker is selected from at least 1 (eg. at least 2, at least 3, at least 4, at least 5, at least 6, at least 7, up to and including all) of the biomarkers: SLC39A8, CIQC, CIQA, MRAS, CIQB, PCOLCE2, KIF2C, TNF, IF144, IFIT1, and RPGRIP1. In one embodiment, the one or more additional biomarker is selected from the group consisting of: SLC39A8, CIQC, CIQA, MRAS, and CIQB.

In one embodiment, the one or more biomarker is SLC39A8, and the one or more additional biomarker is selected from at least 1 (eg. at least 2, at least 3, at least 4, at least 5, at least 6, at least 7, at least 8, at least 9, at least 10, up to and including all) of the biomarkers: CIQC, CIQA, MRAS, TMEM37, CIQB, PCOLCE2, KIF2C, TNF, IF144, IFIT1, and RPGRIP1. In one embodiment, the one or more additional biomarker is selected from the group consisting of: CIQC, CIQA, MRAS, TMEM37, and CIQB.

In one embodiment, the one or more biomarker is KIF2C, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, TMEM37, CIQB, PCOLCE2, TNF, IF144, IFIT1, and RPGRIP1.

In one embodiment, the one or more biomarker is CIQA, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, MRAS, TMEM37, CIQB, PCOLCE2, KIF2C, TNF, IF144, IFIT1, and RPGRIP1. In one embodiment, the one or more additional biomarker is selected from the group consisting of: SLC39A8, CIQC, MRAS, TMEM37, and CIQB.

In one embodiment, the one or more biomarker is CIQC, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQA, MRAS, TMEM37, CIQB, PCOLCE2, KIF2C, TNF, IF144, IFIT1, and RPGRIP1. In one embodiment, the one or more additional biomarker is selected from the group consisting of: SLC39A8, CIQA, MRAS, TMEM37, and CIQB.

In one embodiment, the one or more biomarker is CIQB, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, TMEM37, PCOLCE2, KIF2C, TNF, IF144, IFIT1, and RPGRIP1. In one embodiment, the one or more additional biomarker is selected from the group consisting of: SLC39A8, CIQC, CIQA, MRAS, and TMEM37.

In one embodiment, the one or more biomarker is TNF, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, TMEM37, CIQB, PCOLCE2, KIF2C, IF144, IFIT1, and RPGRIP1.

In one embodiment, the one or more biomarker is IF144, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, MRAS, CIQA, TMEM37, CIQB, PCOLCE2, KIF2C, TNF, IFIT1, and RPGRIP1.

In one embodiment, the one or more biomarker is IFIT1, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, TMEM37, CIQB, PCOLCE2, KIF2C, TNF, IF144, and RPGRIP1.

In one embodiment, the one or more biomarker is RPGRIP1, and the one or more additional biomarker is selected from at least 1 (eg. at least 2, at least 3, at least 4, at least 5, at least 6, at least 7, at least 8, at least 9, at least 10, up to and including all) of the biomarkers: SLC39A8, CIQC, CIQA, MRAS, CIQB, TMEM37, PCOLCE2, KIF2C, TNF, IF144, and IFIT1.

As illustrated in FIG. 3, the present inventors observed that the “abdominal sepsis” biomarkers described herein (MRAS, PCOLCE2, TMEM37, SLC39A8, KIF2C, CIQC, CIQB, CIQA, and TNF) increase in abundance in patients having abdominal sepsis as compared to patients having other systemic inflammatory conditions (such as pulmonary sepsis or SIRS), as well as healthy individuals. The inventors also observed that the biomarkers IF144, IFIT1, and RPGRIP1 decrease in abundance in patients having abdominal sepsis as compared to patients having other systemic inflammatory conditions (such as pulmonary sepsis or SIRS), as well as healthy individuals. These differences in marker abundance can be used to diagnose whether an individual has or is at risk of developing abdominal sepsis.

For example, by comparing the presence and/or amount of markers quantified in a sample obtained from a patient to the presence and/or amount of markers quantified for a reference value (such as a reference value that is representative of a healthy individual (or a population of healthy individuals), a reference value that is representative of an individual having abdominal sepsis (or a population of individuals having abdominal sepsis), a reference value that is representative of an individual having pulmonary sepsis (or a population of individuals having pulmonary sepsis), and/or a reference value that is representative of an individual having SIRS (or a population of individuals having SIRS)), it is possible to diagnose the presence (or absence) of abdominal sepsis in a patient. The method permits classification of the individual as belonging to or not belonging to the reference population (ie. by determining whether the amounts of marker quantified in the individual are statistically similar to the reference population or statistically deviate from the reference population). Hence, classification of the individual's marker profile (ie. the overall pattern of change observed for the markers quantified) as corresponding to the profile derived from a particular reference population is predictive that the individual falls (or does not fall) within the reference population.

In one embodiment, an individual may be diagnosed as having or being at risk of having abdominal sepsis, when the amount of the one or more biomarker is statistically similar to the amount determined for the corresponding reference value representative of an individual (or a population of individuals) having abdominal sepsis. In one embodiment, an individual may be diagnosed as not having or not being at risk of having abdominal sepsis when the amount of the one or more biomarker is statistically similar to the amount determined for the corresponding reference value representative of a healthy individual (or a population of healthy individuals). In one embodiment, an individual may be diagnosed as not having or not being at risk of having abdominal sepsis when the amount of the one or more biomarker is statistically similar to the amount determined for the corresponding reference value representative of an individual having SIRS (or a population of individuals having SIRS). In one embodiment, an individual may be diagnosed as not having or not being at risk of having abdominal sepsis when the amount of the one or more biomarker is statistically similar to the amount determined for the corresponding reference value representative of an individual having pulmonary sepsis (or a population of individuals having pulmonary sepsis).

In one embodiment, an individual may be diagnosed as not having or not being at risk of having abdominal sepsis when the amount of the one or more biomarker quantified statistically deviates from the amount determined for the corresponding reference value representative of an individual (or a population of individuals) having abdominal sepsis. In one embodiment, an individual may be diagnosed as having or being at risk of having abdominal sepsis when the amount of the one or more biomarker quantified statistically deviates from the amount determined for the corresponding reference value representative of a healthy individual (or a population of healthy individuals). In one embodiment, an individual may be diagnosed as having or being at risk of having abdominal sepsis when the amount of the one or more biomarker statistically deviates from the amount determined for the corresponding reference value representative of an individual having SIRS (or a population of individuals having SIRS). In one embodiment, an individual may be diagnosed as having or being at risk of having abdominal sepsis when the amount of the one or more biomarker statistically deviates from the amount determined for the corresponding reference value representative of an individual having pulmonary sepsis (or a population of individuals having pulmonary sepsis).

All embodiments described above for the classification of a patient as having or being at risk of having a systemic inflammatory condition (or as not having or not being at risk of having a systemic inflammatory condition) apply equally to the method for diagnosing whether a patient has or is at risk of having abdominal sepsis. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the method of diagnosing a systemic inflammatory condition in a patient. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having abdominal sepsis (or a population of individuals having abdominal sepsis). In one embodiment, the reference value is representative of an individual having pulmonary sepsis (or a population of individuals having pulmonary sepsis).

As described above for the “method for diagnosing a systemic inflammatory condition in a patient”, the method of the invention may involve the use of multiple separate reference values. For example, the reference value may include one of more (eg. two or more, three or more, or all 4) of the reference values selected from: a reference value that is representative of a healthy individual (or a population of healthy individuals); a reference value that is representative of an individual having SIRS (or a population of individuals having SIRS); and a reference value that is representative of an individual having abdominal sepsis (or a population of individuals having abdominal sepsis); and a reference value that is representative of an individual having pulmonary sepsis (or a population of individuals having pulmonary sepsis).

The present inventors observed that the biomarkers for abdominal sepsis described herein (MRAS, CIQC, CIQB, PCOLCE2, CIQA, TMEM37, SLC39A8, KIF2C, TNF) each increase in abundance in samples obtained from patients having abdominal sepsis, as compared to healthy individuals. Detection of increased levels of these biomarkers in a patient as compared to the levels detected for healthy individuals can thus be used to diagnose the presence of abdominal sepsis. Thus, in one embodiment, when the reference value is representative of a healthy individual (or population of healthy individuals), an increase in the one or more biomarker for abdominal sepsis in the sample obtained from the patient relative to the corresponding reference value representative of a healthy individual, indicates that the patient has abdominal sepsis, or may be at risk of developing abdominal sepsis. Likewise, no increase in the one or more biomarker for abdominal sepsis in the sample obtained from the patient relative to the corresponding reference value representative of a healthy individual, indicates that the patient does not have abdominal sepsis.

For some of the abdominal sepsis biomarkers identified by the present inventors, increased levels of these markers were also observed in patients having other systemic inflammatory conditions (such as pulmonary sepsis and SIRS) as compared to healthy individuals, although typically much bigger increases were observed for these biomarkers in the patients having abdominal sepsis. The accuracy of abdominal sepsis diagnosis can thus be improved by looking for a “minimum” fold change or % change in the levels of the one or more biomarkers as compared to the corresponding reference value that is representative of a healthy individual. The fold increase or % increase may be as defined above for the method for diagnosis of a systemic inflammatory condition.

For example, an increase of at least 50 (eg. at least 55, at least 60, at least 70, at least 80, at least 90, at least 95 at least 100, at least 125, at least 150) fold in PCOLCE2 in the sample obtained from the patient relative to the corresponding reference value, indicates that the patient has, or is at risk of developing abdominal sepsis. In one embodiment, no increase or an increase of less than 50 (eg. less than 55, less than 60, less than 70, less than 80, less than 90, less than 95) fold in PCOLCE2 in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 7 (eg. at least 7.5, at least 8, at least 8.5) fold in TMEM37 in the sample obtained from the patient relative to the corresponding reference value, indicates that the patient has, or is at risk of developing abdominal sepsis. In one embodiment, no increase or an increase of less than 3.5 (eg. less than 4, less than 5, less than 6, less than 7) fold in TMEM37 in the sample obtained from the patient relative to the corresponding reference value, indicates that the patient does not have, or is not at risk of developing abdominal sepsis.

For example, an increase of at least 3 (eg. at least 3.5, at least 4, at least 4.5, at least 5) fold in SLC39A8 in the sample obtained from the patient relative to the corresponding reference value, indicates that the patient has, or is at risk of developing abdominal sepsis. In one embodiment, no increase or an increase of less than 2 (eg. less than 2.5, less than 3, less than 3.5, less than 4) fold in SLC39A8 in the sample obtained from the patient relative to the corresponding reference value, indicates that the patient does not have, or is not at risk of developing abdominal sepsis.

For example, an increase of at least 2.6 (eg. at least 2.7, at least 2.8, at least 2.9, at least 3, at least 3.1) fold in KIF2C in the sample obtained from the patient relative to the corresponding reference value, indicates that the patient has, or is at risk of developing abdominal sepsis. In one embodiment, no increase or an increase of less than 2.6 (eg. less than 2.7, less than 2.8, less than 2.9, less than 3) fold in KIF2C in the sample obtained from the patient relative to the corresponding reference value, indicates that the patient does not have, or is not at risk of developing abdominal sepsis.

For example, an increase of at least 12 (eg. at least 13, at least 14, at least 15, at least 16, at least 17, at least 18, at least 19, at least 20) fold in CIQC in the sample obtained from the patient relative to the corresponding reference value, indicates that the patient has, or is at risk of developing abdominal sepsis. In one embodiment, no increase or an increase of less than 7 (eg. less than 8, less than 9, less than 10, less than 11, less than 12, less than 13, less than 14, less than 15, less than 16, less than 17, less than 18, less than 19, less than 20) fold in CIQC in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 12 (eg. at least 13, at least 14, at least 15, at least 16, at least 17, at least 18, at least 19, at least 20, at least 21, at least 22, at least 23, at least 24 or at least 25) fold in CIQB in the sample obtained from the patient relative to the corresponding reference value, indicates that the patient has, or may be at risk of developing abdominal sepsis. In one embodiment, no increase or an increase of less than 6 (eg. less than 7, less than 8, less than 9, less than 10, less than 11, less than 12, less than 13, less than 14, less than 15, less than 16, less than 17, less than 18, less than 19, less than 20) fold in CIQB in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 10 (eg. at least 11, at least 12, at least 13, at least 14, at least 15, or at least 16) fold in CIQA in the sample obtained from the patient relative to the corresponding reference value, indicates that the patient has, or is at risk of developing abdominal sepsis. In one embodiment, no increase or an increase of less than 4 (eg. less than 5, less than 6, less than 7, less than 8, less than 9, less than 10, less than 11, less than 12, less than 13, less than 14, less than 15, or less than 16) fold in CIQA in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.3 (eg. at least 1.4, or at least 1.5) fold in TNF in the sample obtained from the patient relative to the corresponding reference value, indicates that the patient has, or is at risk of developing abdominal sepsis. In one embodiment, no increase or an increase of less than 1.3 (eg. less than 1.4) fold in TNF in the sample obtained from the patient relative to the corresponding reference value, indicates that the patient does not have, or is not at risk of developing abdominal sepsis. In one embodiment, the patient from which a sample is obtained has been diagnosed as having or being at risk of developing sepsis (eg. using the method of diagnosing sepsis in a patient as described herein and/or using the method of distinguishing between sepsis and SIRS in a patient as described herein).

For example, an increase of at least 1.1 (eg. at least 1.2, at least 1.3, at least 1.4, at least 1.5, or at least 1.6) fold in MRAS in the sample obtained from the patient relative to the corresponding reference value, indicates that the patient has, or is at risk of developing abdominal sepsis. In one embodiment, no increase or an increase of less than 1.1 (less than 1, less than 0.9, less than 0.8, less than 0.7, less than 0.6, less than 0.5) fold in MRAS in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

The present inventors observed that the biomarkers IF144, IFIT1, and RPGRIP1 each decrease in abundance in samples obtained from patients having abdominal sepsis, as compared to healthy individuals. Detection of decreased levels of these biomarkers in a patient as compared to the levels detected for healthy individuals can thus be used to diagnose the presence of abdominal sepsis.

In one embodiment, when the reference value is representative of a healthy individual, a decrease in the one or more biomarker for abdominal sepsis in the sample obtained from the patient relative to the corresponding reference value representative of a healthy individual, indicates that the patient has abdominal sepsis, or may be at risk of developing abdominal sepsis. Likewise, no decrease in the one or more biomarker for abdominal sepsis in the sample obtained from the patient relative to the corresponding reference value representative of a healthy individual, indicates that the patient does not have abdominal sepsis.

For the biomarkers IFIT1 and RPGRIP1, decreased levels of these markers were also observed in patients having other systemic inflammatory conditions (such as pulmonary sepsis and SIRS) as compared to healthy individuals, although typically much bigger decreases were observed for these biomarkers in the patients having abdominal sepsis. The accuracy of abdominal sepsis diagnosis can thus be improved by looking for a “minimum” fold decrease or % decrease in the levels of the one or more biomarkers as compared to the corresponding reference value that is representative of a healthy individual. The fold decrease or % decrease may be as defined above for the method for diagnosis of a systemic inflammatory condition.

For example, a decrease of at least 0.5 (eg. at least 0.6, at least 0.7, at least 0.8, at least 0.9, or at least 1) fold in IF144 in the sample obtained from the patient relative to the corresponding reference value, indicates that the patient has, or is at risk of developing abdominal sepsis. In one embodiment, no decrease in IF144 in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, a decrease of at least 2.5 (eg. at least 2.6, at least 2.7, at least 2.8, at least 2.9, or at least 3) fold in IFIT1 in the sample obtained from the patient relative to the corresponding reference value, indicates that the patient has, or is at risk of developing abdominal sepsis. In one embodiment, no decrease or a decrease of less than 1.9 (eg. less than 2, less than 2.1, less than 2.2, less than 2.3, less than 2.4, less than 2.5) fold in IFIT1 in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, a decrease of at least 1.75 (eg. at least 1.8, at least 1.9, at least 2, at least 2.5, at least 3, at least 3.5, at least 4, at least 4.5, or at least 5) fold in RPGRIP1 in the sample obtained from the patient relative to the corresponding reference value, indicates that the patient has, or is at risk of developing abdominal sepsis. In one embodiment, no decrease or a decrease of less than 1.4 (eg. less than 1.5, less than 1.6, less than 1.7, less than 1.8, less than 1.9, less than 2) fold in RPGRIP1 in the sample obtained from the patient relative to the corresponding reference value, indicates that the patient does not have, or is not at risk of developing abdominal sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

As described herein, the present inventors observed that the levels of the one or more sepsis biomarkers (MRAS, PCOLCE2, TMEM37, SLC39A8, KIF2C, CIQC, CIQB, CIQA, and TNF) were elevated in patients having abdominal sepsis as compared to patients having other systemic inflammatory conditions such as pulmonary sepsis or SIRS (with the exception of TNF which is increased in abundance as compared to patients having pulmonary sepsis only). Detection of increased levels of these biomarkers in a patient as compared to the levels detected for patients having one or more of these other systemic inflammatory conditions can thus be used to diagnose the presence of abdominal sepsis.

Thus, in one embodiment, an increase in the one or more biomarker (and/or one or more additional biomarker) in the sample obtained from the patient relative to the corresponding reference value representative of an individual having pulmonary sepsis, indicates that the patient has or is at risk of developing abdominal sepsis. Likewise, no increase in the one or more biomarker (and/or one or more additional biomarker) in the sample obtained from the patient relative to the corresponding reference value representative of an individual having pulmonary sepsis, indicates that the patient does not have abdominal sepsis.

In one embodiment, an increase in the one or more biomarker (and/or one or more additional biomarker) in the sample obtained from the patient relative to the corresponding reference value representative of an individual having SIRS, indicates that the patient has or is at risk of developing abdominal sepsis. Likewise, no increase in the one or more biomarker (and/or one or more additional biomarker) in the sample obtained from the patient relative to the corresponding reference value representative of an individual having SIRS, indicates that the patient does not have abdominal sepsis.

In one embodiment, the patient may be diagnosed as having abdominal sepsis, or being at risk of developing abdominal sepsis, when the one or more biomarker (or the one or more additional biomarker) increases by at least 1 (e.g. at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value representative of an individual having pulmonary sepsis and/or an individual having SIRS.

As described herein, the present inventors observed that the levels of the biomarkers IF144, IFIT1, and RPGRIP1 were decreased in patients having abdominal sepsis as compared to patients having other systemic inflammatory conditions such as pulmonary sepsis or SIRS. Detection of decreased levels of these biomarkers in a patient as compared to the levels detected for patients having one or more of these other systemic inflammatory conditions can thus be used to diagnose the presence of abdominal sepsis.

Thus, in one embodiment, a decrease in the one or more biomarker (and/or one or more additional biomarker) in the sample obtained from the patient relative to the corresponding reference value representative of an individual having pulmonary sepsis, indicates that the patient has or is at risk of developing abdominal sepsis. Likewise, no decrease in the one or more biomarker (and/or one or more additional biomarker) in the sample obtained from the patient relative to the corresponding reference value representative of an individual having pulmonary sepsis, indicates that the patient does not have abdominal sepsis.

In one embodiment, a decrease in the one or more biomarker (and/or one or more additional biomarker) in the sample obtained from the patient relative to the corresponding reference value representative of an individual having SIRS, indicates that the patient has or is at risk of developing abdominal sepsis. Likewise, no decrease in the one or more biomarker (and/or one or more additional biomarker) in the sample obtained from the patient relative to the corresponding reference value representative of an individual having SIRS, indicates that the patient does not have abdominal sepsis.

In one embodiment, the patient may be diagnosed as having abdominal sepsis, or being at risk of developing abdominal sepsis, when the one or more biomarker (or the one or more additional biomarker) decreases by at least 0.1 (e.g. at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value representative of an individual having pulmonary sepsis and/or an individual having SIRS.

In a related aspect, the present invention also provides the use of one or more of: SLC39A8, CIQC, CIQA, MRAS, TMEM37, CIQB, PCOLCE2, KIF2C, TNF, IF144, IFIT1, and RPGRIP1 as a biomarker for abdominal sepsis. In one embodiment, the one or more biomarker is selected from the group consisting of: SLC39A8, CIQC, CIQA, MRAS, TMEM37, CIQB, PCOLCE2, KIF2C, and TNF. In one embodiment, the one or more biomarker is selected from the group consisting of: SLC39A8, CIQC, CIQA, MRAS, TMEM37, CIQB, PCOLCE2, and KIF2C. In one embodiment, the one or more biomarker is selected from the group consisting of: SLC39A8, CIQC, CIQA, MRAS, TMEM37, and CIQB. In one embodiment, the one or more biomarker is selected from the group consisting of: SLC39A8, CIQC, and CIQA. In one embodiment, the use is of the one or more biomarker in the diagnosis of abdominal sepsis in a patient.

All embodiments described above for the method of diagnosing abdominal sepsis in a patient apply equally to the ‘use’ of the invention described herein. This includes all embodiments relating to the terms “abdominal sepsis”, “patient”, “sample”, and “the one or more biomarker”.

As discussed herein, the present inventors have also observed that the levels of HCAR2, CXCR1, DISC1, EPSTI1, and IF144, are elevated in patients having pulmonary sepsis, and are thus suitable for use as biomarkers for pulmonary sepsis (see Table 3).

The present invention therefore also provides a method for diagnosing pulmonary sepsis in a patient, comprising:

(i) determining the presence and/or amount of one or more biomarker in a sample obtained from a patient, wherein the one or more biomarker is selected from the group consisting of: HCAR2, CXCR1, DISC1, EPSTI1, and IF144;
(ii) comparing the presence and/or amount of the one or more biomarker determined in said sample in (i) to a corresponding reference value; and thereby determining whether the patient has or is at risk of developing pulmonary sepsis.

All embodiments described above for the “method for diagnosing a systemic inflammatory condition in a patient” apply equally to the “method for diagnosing pulmonary sepsis in a patient”. This includes all embodiments relating to the “sample”, “patient”, “biomarker”, and “reference value”, and all embodiments relating to the step for “determining the presence and/or amount of one or more biomarker in a sample” and for the “comparison” step for determining whether the patient has or is at risk of developing pulmonary sepsis.

As used herein, the phrase “diagnosis of pulmonary sepsis in a patient” means determining whether the patient has or is risk of developing pulmonary sepsis. The term “pulmonary sepsis” is as described above for the “method for diagnosing a systemic inflammatory condition in a patient”.

The “patient” for which diagnosis is performed is as defined above for the “method for diagnosing a systemic inflammatory condition in a patient”, and the “method for diagnosing sepsis in a patient”. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having a systemic inflammatory condition using the method described herein. In one embodiment, the patient is suspected of having or being at risk of developing sepsis. In one embodiment, the patient has been diagnosed as having or being at risk of developing sepsis (eg. using the method of diagnosing sepsis in a patient as described herein and/or using the method of distinguishing between sepsis and SIRS in a patient as described herein). In one embodiment, the patient is suspected of having or being at risk of developing pulmonary sepsis.

The “sample” obtained from the patient is as defined above for the “method for diagnosing a systemic inflammatory condition in a patient”, including all embodiments relating to the time point at which the sample is obtained. All embodiments of the “sample” described above for the method for diagnosing sepsis also apply to the method for diagnosing pulmonary sepsis.

The “one or more biomarker” of the invention is as defined above for the “method for diagnosing a systemic inflammatory condition in a patient”.

In one embodiment, the one or more biomarker may be selected from the group consisting of: HCAR2, CXCR1, DISC1, EPSTI1, and IF144. For example, the one or more biomarker may be selected from the group consisting of: EPSTI1 and DISC1. For example, the one or more biomarker may be selected from the group consisting of: CXCR1, HCAR2, and IF144.

The present inventors observed that the biomarkers CXCR1, HCAR2, and IF144 are elevated in patients having pulmonary sepsis as compared to patients having abdominal sepsis. However, these biomarkers were also observed as being elevated in patients having SIRS, and thus these biomarkers are particularly useful for diagnosing pulmonary sepsis in patients already diagnosed as having sepsis (eg. using the methods described herein for diagnosis of sepsis, or using the method described herein for distinguishing between abdominal sepsis and pulmonary sepsis). Thus, when the patient has already been diagnosed as having sepsis, the one or more biomarker may be selected from the group consisting of: CXCR1, HCAR2, and IF144.

Each of the biomarkers of pulmonary sepsis may be used alone, or in combination with any of the pulmonary sepsis biomarkers in the method of the invention. For example, any combination of 1 or more, 2 or more, 3 or more, 4 or more, or up to and including all of the pulmonary sepsis biomarkers may be used to diagnose pulmonary sepsis in a patient according to the method of the invention.

In one embodiment, the one or more biomarker is HCAR2. In one embodiment, the one or more biomarker is CXCR1. In one embodiment, the one or more biomarker is DISC1. In one embodiment, the one or more biomarker is EPSTI1. In one embodiment, the one or more biomarker is IF144.

In one embodiment, any combination of 1 or more (eg. 2 or more, 3 or more, 4 or more, or all 5) of the biomarkers selected from the group consisting of: HCAR2, CXCR1, DISC1, EPSTI1, and IF144, may be used to diagnose pulmonary sepsis in a patient. In one embodiment, any combination of 1 or more (eg. 2 or more, or all 3) of the biomarkers selected from the group consisting of: CXCR1, HCAR2, and IF144 may be used to diagnose pulmonary sepsis in a patient. In one embodiment, any combination of 1 or more (eg. or both) of the biomarkers selected from the group consisting of: EPSTI1 and DISC1, may be used to diagnose pulmonary sepsis in a patient.

For example, the following combinations of pulmonary sepsis biomarkers may be used to diagnose pulmonary sepsis: (i) EPSTI1 and HCAR2; (ii) EPSTI1 and DISC1; (iii) EPSTI1 and CXCR1; (iv) EPSTI1 and IF144; (v) DISC1 and CXCR1; (vi) DISC1 and HCAR2; (vii) DISC1 and IF144; (viii) CXCR1 and HCAR2; (ix) CXCR1 and IF144; (x) HCAR2 and IF144.

As described in Example 2, a sub-set of the biomarkers tested (HCAR2, CXCR1, DISC1) was observed to provide particularly accurate diagnosis of pulmonary sepsis (see the ROC curve data in Example 2). In one embodiment, any combination of 1 or more (eg. 2 or more, or all 3) of the biomarkers selected from the group consisting of: HCAR2, CXCR1, DISC1, may be used to diagnose pulmonary sepsis in a patient. For example, the combination of HCAR2, CXCR1, and DISC1 may be used to diagnose abdominal sepsis in a patient. For example, the combination of HCAR2 and CXCR1 may be used to diagnose abdominal sepsis in a patient.

One or more additional biomarker for pulmonary sepsis may also be used in the diagnosis of pulmonary sepsis according to the method of the invention. Any combination of the one or more additional biomarker may be used in combination with the one or more biomarker of the invention. For example at least 1, at least 2, at least 3, or all 4 additional biomarkers for pulmonary sepsis may be used in combination with the one or more biomarker of the invention (as described herein). Typically, the one or more additional biomarker is selected from the group consisting of: HCAR2, CXCR1, DISC1, EPSTI1, and IF144. In one embodiment, one or more additional biomarker is selected from the group consisting of: HCAR2, CXCR1, and DISC1.

In one embodiment, the one or more biomarker is HCAR2, and the one or more additional biomarker is selected from at least 1 (eg. at least 2, at least 3, up to and including all) of the biomarkers: EPSTI1, DISC1, CXCR1, and IF144. In one embodiment, the one or more additional biomarker is selected from CXCR1 and/or DISC1.

In one embodiment, the one or more biomarker is CXCR1, and the one or more additional biomarker is selected from at least 1 (eg. at least 2, at least 3, up to and including all) of the biomarkers: EPSTI1, DISC1, HCAR2, and IF144. In one embodiment, the one or more additional biomarker is selected from HCAR2 and/or DISC1.

In one embodiment, the one or more biomarker is DISC1, and the one or more additional biomarker is selected from at least 1 (eg. at least 2, at least 3, up to and including all) of the biomarkers: EPSTI1, HCAR2, CXCR1, and IF144. In one embodiment, the one or more additional biomarker is selected from HCAR2 and/or CXCR1.

In one embodiment, the one or more biomarker is EPSTI1, and the one or more additional biomarker is selected from at least 1 (eg. at least 2, at least 3, up to and including all) of the biomarkers: DISC1, CXCR1, HCAR2, and IF144.

In one embodiment, the one or more biomarker is IF144, and the one or more additional biomarker is selected from at least 1 (eg. at least 2, at least 3, up to and including all) of the biomarkers: EPSTI1, DISC1, CXCR1, and HCAR2.

As illustrated in FIG. 3, the present inventors observed that the “pulmonary sepsis” biomarkers described herein increased in abundance in patients having pulmonary sepsis as compared to patients having other systemic inflammatory conditions (such as abdominal sepsis or SIRS), as well as healthy individuals. These differences in marker abundance can be used to diagnose whether an individual has or is at risk of developing pulmonary sepsis.

For example, by comparing the amount of markers quantified in a sample obtained from a patient to the amount of markers quantified for a reference value (such as a reference value that is representative of a healthy individual (or a population of healthy individuals), a reference value that is representative of an individual having abdominal sepsis (or a population of individuals having abdominal sepsis), a reference value that is representative of an individual having pulmonary sepsis (or a population of individuals having pulmonary sepsis), and/or a reference value that is representative of an individual having SIRS (or a population of individuals having SIRS)), it is possible to diagnose the presence (or absence) of pulmonary sepsis in a patient. The method permits classification of the individual as belonging to or not belonging to the reference population (ie. by determining whether the amounts of marker quantified in the individual are statistically similar to the reference population or statistically deviate from the reference population). Hence, classification of the individual's marker profile (ie. the overall pattern of change observed for the markers quantified) as corresponding to the profile derived from a particular reference population is predictive that the individual falls (or does not fall) within the reference population.

In one embodiment, an individual may be diagnosed as having or being at risk of having pulmonary sepsis, when the amount of the one or more biomarker is statistically similar to the amount determined for the corresponding reference value representative of an individual (or a population of individuals) having pulmonary sepsis. In one embodiment, an individual may be diagnosed as not having or not being at risk of having pulmonary sepsis when the amount of the one or more biomarker is statistically similar to the amount determined for the corresponding reference value representative of a healthy individual (or a population of healthy individuals). In one embodiment, an individual may be diagnosed as not having or not being at risk of having pulmonary sepsis when the amount of the one or more biomarker is statistically similar to the amount determined for the corresponding reference value representative of an individual having abdominal sepsis (or a population of individuals having abdominal sepsis). In one embodiment, an individual may be diagnosed as not having or not being at risk of having pulmonary sepsis when the amount of the one or more biomarker is statistically similar to the amount determined for the corresponding reference values representative of an individual having SIRS (or a population of individuals having SIRS).

In one embodiment, an individual may be diagnosed as not having or not being at risk of having pulmonary sepsis when the amount of the one or more biomarker statistically deviates from the amount determined for the corresponding reference value representative of an individual (or a population of individuals) having pulmonary sepsis. In one embodiment, an individual may be diagnosed as having or being at risk of having pulmonary sepsis when the amount of the one or more biomarker statistically deviates from the amount determined for the corresponding reference values representative of a healthy individual (or a population of healthy individuals). In one embodiment, an individual may be diagnosed as having or being at risk of having pulmonary sepsis when the amount of the one or more biomarkers statistically deviates from the amount determined for the corresponding reference value representative of an individual having abdominal sepsis (or a population of individuals having abdominal sepsis). In one embodiment, an individual may be diagnosed as having or being at risk of having pulmonary sepsis when the amount of the one or more biomarker statistically deviates from the amount determined for the corresponding reference values representative of an individual having SIRS (or a population of individuals having SIRS).

All embodiments described above for the classification of a patient as having or being at risk of having a systemic inflammatory condition (or as not having or not being at risk of having a systemic inflammatory condition) apply equally to the method for diagnosing whether a patient has or is at risk of having pulmonary sepsis. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the method of diagnosing a systemic inflammatory condition in a patient. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having abdominal sepsis (or a population of individuals having abdominal sepsis). In one embodiment, the reference value is representative of an individual having pulmonary sepsis (or a population of individuals having pulmonary sepsis).

As described above for the “method for diagnosing a systemic inflammatory condition in a patient”, the method of the invention may involve the use of multiple separate reference values. For example, the reference value may include one of more (eg. two or more, three of more, or all 4) of the reference values selected from: a reference value that is representative of a healthy individual (or a population of healthy individuals); a reference value that is representative of an individual having SIRS (or a population of individuals having SIRS); a reference value that is representative of an individual having abdominal sepsis (or a population of individuals having abdominal sepsis), and a reference value that is representative of an individual having pulmonary sepsis (or a population of individuals having pulmonary sepsis).

The present inventors observed that the pulmonary sepsis biomarkers EPSTI1, DISC1, CXCR1, HCAR2 and IF144 each increase in abundance in samples obtained from patients having pulmonary sepsis, as compared to healthy individuals. Detection of increased levels of these biomarkers in a patient as compared to the levels detected for healthy individuals can thus be used to diagnose the presence of pulmonary sepsis.

In one embodiment, an increase in the one or more biomarker for pulmonary sepsis in the sample obtained from the patient relative to the corresponding reference value representative of a healthy individual, indicates that the patient has pulmonary sepsis, or is at risk of developing pulmonary sepsis. Likewise, no increase in the one or more biomarker for pulmonary sepsis in the sample obtained from the patient relative to the corresponding reference value representative of a healthy individual, indicates that the patient does not have pulmonary sepsis.

For some of the pulmonary sepsis biomarkers identified by the present inventors (DISC1, CXCR1, HCAR2, and IF144), increased levels of these markers were also observed in patients having other systemic inflammatory conditions (abdominal sepsis or SIRS) as compared to healthy individuals, although much bigger increases were observed for these biomarkers in the patients having pulmonary sepsis. The accuracy of pulmonary sepsis diagnosis can thus be improved by looking for a “minimum” fold increase or % increase in the levels of the one or more biomarkers as compared to the corresponding reference value that is representative of a healthy individual. The fold increase or % increase may be as defined above for the method for diagnosis of a systemic inflammatory condition.

For example, an increase of at least 1 (eg. at least 1.05, at least 1.1, at least 1.15. at least 1.2, at least 1.25, at least 1.3) fold in EPSTI1 in the sample obtained from the patient relative to the corresponding reference value, indicates that the patient has, or is at risk of developing pulmonary sepsis. In one embodiment, no increase or an increase of less than 1 (eg. less than 1.05, less than 1.1) fold in EPSTI1 in the sample obtained from the patient relative to the corresponding reference value, indicates that the patient does not have, or is not at risk of developing pulmonary sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 1.8 (eg. at least 2, at least 2.1, at least 2.2, at least 2.3, at least 2.4, at least 2.5, at least 3) fold in DISC1 in the sample obtained from the patient relative to the corresponding reference value, indicates that the patient has, or is at risk of developing pulmonary sepsis. In one embodiment, no increase or an increase of less than 1.3 (eg. less than 1.4, less than 1.5, less than 1.6, less than 1.7, less than 1.8, less than 1.9, less than 2) fold in DISC1 in the sample obtained from the patient relative to the corresponding reference value, indicates that the patient does not have, or is not at risk of developing pulmonary sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

For example, an increase of at least 3.5 (eg. at least 3.6, at least 3.7, at least 3.8, at least 3.9, at least 4) fold in CXCR1 in the sample obtained from the patient relative to the corresponding reference value, indicates that the patient has, or is at risk of developing pulmonary sepsis. In one embodiment, no increase or an increase of less than 3.5 (eg. less than 3, less than 2.5, less than 2) fold in CXCR1 in the sample obtained from the patient relative to the corresponding reference value, indicates that the patient does not have, or is not at risk of developing pulmonary sepsis. In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility. In one embodiment, the patient from which a sample is obtained has been diagnosed as having or being at risk of developing sepsis (eg. using the method of diagnosing sepsis in a patient as described herein and/or using the method of distinguishing between sepsis and SIRS in a patient as described herein).

For example, an increase of at least 1.4 (eg. at least 1.5, at least 1.6, at least 1.7, at least 1.8, at least 1.9, at least 2) fold in HCAR2 in the sample obtained from the patient relative to the corresponding reference value, indicates that the patient has, or is at risk of developing pulmonary sepsis. In one embodiment, no increase or an increase of less than 1.5 (eg. less than 1.4, less than 1.3, less than 1.2, less than 1.1, less than 1) fold in HCAR2 in the sample obtained from the patient relative to the corresponding reference value, indicates that the patient does not have, or is not at risk of developing pulmonary sepsis. In one embodiment, the patient from which a sample is obtained has been diagnosed as having or being at risk of developing sepsis (eg. using the method of diagnosing sepsis in a patient as described herein and/or using the method of distinguishing between sepsis and SIRS in a patient as described herein).

For example, an increase of at least 1.4 (eg. at least 1.5, at least 1.6, at least 1.7, at least 1.8, at least fold 1.9, or at least 2) fold in IF144 in the sample obtained from the patient relative to the corresponding reference value, indicates that the patient has, or is at risk of developing pulmonary sepsis. In one embodiment, no increase or an increase of less than 1.7 (eg. less than 1.6, less than 1.5, less than 1.4, less than 1.3, less than 1.2, less than 1.1) fold in IF144 in the sample obtained from the patient relative to the corresponding reference value, indicates that the patient does not have, or is not at risk of developing pulmonary sepsis. In one embodiment, the patient from which a sample is obtained has been diagnosed as having or being at risk of developing sepsis (eg. using the method of diagnosing sepsis in a patient as described herein and/or using the method of distinguishing between sepsis and SIRS in a patient as described herein). In one embodiment, when detecting this level of fold change in the biomarker, the method is performed using a sample obtained from a patient up to 24 (eg. up to 36, up to 48, up to 72, or up to 96) hours after the patient presents with one or more clinical symptoms of a systemic inflammatory condition, or is admitted to a medical care facility.

As described herein, the present inventors observed that the levels of the one or more “pulmonary sepsis” biomarkers were elevated in patients having pulmonary sepsis as compared to patients having other systemic inflammatory conditions such as abdominal sepsis or SIRS. Detection of increased levels of these biomarkers in a patient as compared to the levels detected for patients having one or more of these other systemic inflammatory conditions can thus be used to diagnose the presence of pulmonary sepsis.

In one embodiment, when the reference value is representative of an individual having abdominal sepsis, an increase in the one or more biomarker (and/or one or more additional biomarker) in the sample obtained from the patient relative to the corresponding reference value indicates that the patient has or is at risk of developing pulmonary sepsis. Likewise, no increase in the one or more biomarker (and/or one or more additional biomarker) in the sample obtained from the patient relative to the corresponding reference value, indicates that the patient does not have pulmonary sepsis.

In one embodiment, when the reference value is representative of an individual having SIRS, an increase in the one or more biomarker (and/or one or more additional biomarker) in the sample obtained from the patient relative to the corresponding reference value, indicates that the patient has or is at risk of developing pulmonary sepsis. Likewise, no increase in the one or more biomarker (and/or one or more additional biomarker) in the sample obtained from the patient relative to the corresponding reference value, indicates that the patient does not have pulmonary sepsis.

In one embodiment, the patient may be diagnosed as having pulmonary sepsis, or being at risk of developing pulmonary sepsis, when the one or more biomarker (or the one or more additional biomarker) increases by at least 0.1 (e.g. at least 0.2, at least 0.3, at least 0.4, at least 0.5, at least 0.6, at least 0.7, at least 0.9, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, or at least 50) fold in the sample obtained from the patient relative to the corresponding reference value representative of an individual having abdominal sepsis and/or an individual having SIRS.

In a related aspect, the present invention also provides the use of one or more of: HCAR2, CXCR1, DISC1, EPSTI1, and IF144 as a biomarker for pulmonary sepsis. In one embodiment, the one or more biomarker is selected from the group consisting of: HCAR2, CXCR1, and DISC1. In one embodiment, the one or more biomarker is selected from the group consisting of: HCAR2, and CXCR1. In one embodiment, the present invention provides the use of a combination of HCAR2, CXCR1, and optionally DISC1 as biomarkers for pulmonary sepsis. In one embodiment, the use is of the one or more biomarker in the diagnosis of pulmonary sepsis in a patient.

All embodiments described above for the method of diagnosing pulmonary sepsis in a patient apply equally to the ‘use’ of the invention described herein. This includes all embodiments relating to the terms “pulmonary sepsis”, “patient”, “sample”, and “the one or more biomarker”.

The method for diagnosis of sepsis, the method for diagnosis of abdominal sepsis and/or the method for diagnosis of pulmonary sepsis as described herein can be used in a decision tree process to investigate the health of a patient having or suspected of having a systemic inflammatory condition. For example, the method for diagnosis of sepsis, the method for diagnosis of abdominal sepsis and/or the method for diagnosis pulmonary sepsis as described herein in a patient can be performed before, after, or in addition to any of the methods of the invention described herein.

In one embodiment, the method of the invention for diagnosing sepsis in a patient (as described herein) can be performed subsequent to (or in addition to) the method for diagnosing whether a patient has a systemic inflammatory condition (as described herein). If the patient tests positive for a systemic inflammatory condition (using the method for diagnosing whether a patient has a systemic inflammatory condition), they may be tested for sepsis using the diagnostic method described herein. In one embodiment, the above combination of methods are performed as described, and if the patient tests positive for sepsis, the patient may be further tested for abdominal sepsis and/or pulmonary sepsis using the diagnostic methods of the invention described herein, so as to determine whether the patient has or is at risk of developing abdominal and/or pulmonary sepsis.

In one embodiment, the method of the invention for diagnosing sepsis in a patient (as described herein) can be performed subsequent to (or in addition to) the method for distinguishing between sepsis and SIRS in a patient (as described herein). If the patient tests positive for sepsis using the distinguishing method of the invention, they may be tested for sepsis using the diagnostic method described herein, so as to further confirm whether the patient has or is at risk of developing sepsis. In one embodiment, the above combination of methods are performed as described, and if the patient tests positive for sepsis, the patient may be further tested for abdominal sepsis and/or pulmonary sepsis using the diagnostic methods of the invention described herein, so as to determine whether the patient has or is at risk of developing abdominal and/or pulmonary sepsis.

In one embodiment, the method for diagnosis of sepsis may be performed subsequent to (or in addition to) the method for diagnosing whether a patient has a systemic inflammatory condition (as described herein), and the method for distinguishing between sepsis and SIRS in a patient (as described herein). For example, the patient may be tested using the method for diagnosing whether a patient has a systemic inflammatory condition (as described herein). If the patient tests positive for a systemic inflammatory condition, they may be tested using the distinguishing method of the invention (as described herein) to determine whether them patient has or is at risk of developing sepsis and/or SIRS. If the patient tests positive for sepsis using the distinguishing method of the invention, they may be tested for sepsis using the diagnostic method described herein, so as to further confirm the diagnosis. In one embodiment, the above combination of methods are performed as described, and if the patient tests positive for sepsis, the patient may be further tested for abdominal sepsis and/or pulmonary sepsis using the diagnostic methods of the invention described herein, so as to determine whether the patient has or is at risk of developing abdominal and/or pulmonary sepsis.

In one embodiment, the method for diagnosing abdominal sepsis and/or pulmonary sepsis in a patient (as described herein) may be performed subsequent to (or in addition to) the method for diagnosing whether a patient has a systemic inflammatory condition (as described herein). If the patient tests positive for a systemic inflammatory condition (using the method of the invention for diagnosing whether a patient has a systemic inflammatory condition), they may be tested for abdominal sepsis and/or pulmonary sepsis using the diagnostic methods described herein to determine whether the patient has or is at risk of developing abdominal sepsis and/or pulmonary sepsis.

In one embodiment, the method of the invention for diagnosing abdominal sepsis and/or pulmonary sepsis in a patient (as described herein) can be performed subsequent to (or in addition to) the method for distinguishing between sepsis and SIRS in a patient (as described herein). If the patient tests positive for sepsis using the distinguishing method of the invention, they may be tested for abdominal sepsis and/or pulmonary sepsis using the diagnostic methods described herein, so as to further determine whether the patient has or is at risk of developing abdominal sepsis and/or pulmonary sepsis.

In one embodiment, the method for diagnosing abdominal sepsis and/or pulmonary sepsis in a patient (as described herein) may be performed subsequent to (or in addition to) the method for diagnosing whether a patient has a systemic inflammatory condition (as described herein), and the method for distinguishing between sepsis and SIRS in a patient (as described herein). For example, the patient may be tested using the method for diagnosing whether a patient has a systemic inflammatory condition (as described herein). If the patient tests positive for a systemic inflammatory condition, they may be tested using the distinguishing method of the invention (as described herein) to determine whether the patient has or is at risk of developing sepsis and/or SIRS. If the patient tests positive for sepsis using the distinguishing method of the invention, they may be tested for abdominal sepsis and/or pulmonary sepsis using the diagnostic methods described herein, so as to determine whether the patient has or is at risk of developing abdominal sepsis and/or pulmonary sepsis.

Furthermore, in one embodiment, each of above combination of methods may be performed as described, and if the patient tests positive for a systemic inflammatory condition, they may be tested for SIRS using the diagnostic method described herein, in addition to being tested for sepsis, abdominal sepsis and/or pulmonary sepsis using the methods described herein.

The above described combination of methods may be performed in parallel to determine the disease status of a patient by simultaneously (or substantially simultaneously) investigating the expression of all the biomarkers in a sample obtained from the patient, and determining whether the patient has or is at risk of having sepsis (such as abdominal or pulmonary sepsis).

When performing these different methods in a decision tree process, the sample used in each step of the method may be the same sample obtained from the patient (as described herein). When the method comprises multiple quantification steps, all the steps may be performed at the same time (e.g. in parallel) and/or using the same sample.

### Methods for Distinguishing Between Different Types of Systemic Inflammatory Conditions

Sepsis and SIRS are both systemic inflammatory conditions associated with overlapping clinical symptoms. Distinguishing between these conditions is important, because different treatments are required for the two conditions. As described herein, the present inventors have identified a set of biomarkers that is predictive of sepsis and a separate set of biomarkers that is predictive of SIRS in patients. Using these distinct sets of biomarkers, the present inventors have developed a rapid and sensitive way to distinguish between SIRS and sepsis in a patient by quantifying one or more biomarker for sepsis and/or one or more biomarker for SIRS in a sample obtained from a patient, so as to determine whether the patient has a biomarker profile that is predictive of sepsis or SIRS.

The present invention therefore provides a method for distinguishing between sepsis and SIRS in a patient, comprising:

(i) determining the presence and/or amount of one or more biomarker for sepsis (as described herein), and/or one or more biomarker for SIRS (as described herein) in a sample obtained from a patient,
(ii) comparing the presence and/or amount of the one or more biomarker for sepsis and/or the one or more biomarker for SIRS determined in said sample in (i) to a corresponding reference value; and thereby determining whether the patient has sepsis and/or SIRS.

As illustrated in Example 2, the distinguishing method of the invention can be performed using only one or more of the sepsis biomarker described herein, or only one or more of the SIRS biomarkers described herein. These biomarkers can be used on their own to distinguish sepsis and SIRS because their expression correlates with the patient's disease condition (i.e. the presence and/or amount of these biomarkers depends on whether a patient has sepsis or SIRS or is healthy). Determining the presence and/or amount of either of these biomarkers and comparing this to a corresponding reference value (such as a reference value that is representative of a healthy individual, a sepsis patient and/or a SIRS patient) therefore allows the disease status of the patient to be determined.

In one embodiment, the present invention provides a method for distinguishing between sepsis and SIRS in a patient, comprising:

(i) determining the presence and/or amount of one or more biomarker for sepsis (as described herein) in a sample obtained from a patient,
(ii) comparing the presence and/or amount of the one or more biomarker for sepsis determined in said sample in (i) to a corresponding reference value; and thereby determining whether the patient has sepsis and/or SIRS.

In one embodiment, the present invention provides a method for distinguishing between sepsis and SIRS in a patient, comprising:

(i) determining the presence and/or amount of one or more biomarker for SIRS (as described herein) in a sample obtained from a patient,
(ii) comparing the presence and/or amount of the one or more biomarker for SIRS determined in said sample in (i) to a corresponding reference value; and thereby determining whether the patient has sepsis and/or SIRS.

Alternatively, the one or more biomarker for sepsis may used in combination with the one or more biomarker for SIRS to distinguish between sepsis and SIRS in a patient.

In one embodiment, the present invention provides a method for distinguishing between sepsis and SIRS in a patient, comprising:

(i) determining the presence and/or amount of one or more biomarker for sepsis (as described herein), and one or more biomarker for SIRS (as described herein) in a sample obtained from a patient,
(ii) comparing the presence and/or amount of the one or more biomarker for sepsis determined in said sample in (i) to a corresponding reference value;
(iii) comparing the presence and/or amount of the one or more biomarker for SIRS in said sample in (i) to a corresponding reference value; and thereby determining whether the patient has sepsis and/or SIRS.

All embodiments described above for the “method for diagnosing a systemic inflammatory condition”, the “method for diagnosing SIRS”, and the “method for diagnosing sepsis” (including the methods for diagnosing abdominal sepsis and pulmonary sepsis) apply equally to the “method for distinguishing between sepsis and SIRS in a patient”. This includes all embodiments relating to the “sample”, “patient”, “biomarker”, and “reference value”, and all embodiments relating to the step for “determining the presence and/or amount of one or more biomarker in a sample” and for the “comparison” step for making a conclusion about the disease status of the patient.

As used herein, “distinguishing between sepsis and SIRS” means to determine whether a patient has or is at risk of developing sepsis and/or SIRS. For example, it may involve determining whether a patient has or is at risk of developing sepsis or SIRS. For example, it may involve determining whether a patient has or is at risk of developing sepsis and SIRS. This may involve distinguishing between a group (ie. one or more) of patients having sepsis and a group (ie. one or more) of patients having SIRS. In one embodiment, this may involve diagnosing or determining whether a patient has or is at risk of developing one or more systemic inflammatory condition selected from: sepsis and SIRS.

The systemic inflammatory conditions “sepsis” and “SIRS” are as described above for the “method for diagnosing a systemic inflammatory condition in a patient”.

The “patient” for which diagnosis is performed is as described above for the “method for diagnosing a systemic inflammatory condition in a patient”. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having or being at risk of developing a systemic inflammatory condition using the method described herein for diagnosis of a systemic inflammatory condition in a patient. In one embodiment, the patient is suspected of having or being at risk of developing sepsis and/or SIRS. In one embodiment, the patient is suspected of having or being at risk of developing sepsis. In one embodiment, the patient is suspected of having or being at risk of developing SIRS.

The “sample” obtained from the patient is as defined above for the “method for diagnosing a systemic inflammatory condition in a patient”, the “method for diagnosing SIRS in a patient” and the “method for diagnosing sepsis in a patient”, including all embodiments relating to the time point at which the sample is obtained.

The “one or more biomarker” of the invention is as defined above for the “method for diagnosing a systemic inflammatory condition in a patient”.

The “one or more biomarker for sepsis” may be as defined above for the method for diagnosing sepsis in a patient, and includes any of the one or more sepsis biomarkers described herein (with or without the one or more additional biomarker) and further includes any of the combinations of sepsis biomarkers described herein.

For example, the one or more biomarker for sepsis may be selected from the group consisting of: ITGB3, ITGA2B, MYL9, LCN2, TREML1, LCN15, CMTM5, PPBP, PF4, KIF2C, MAP1A, SELP, NEXN, NLRC4, and CLEC1B. In a further example, the one or more biomarker for sepsis may be selected from the group consisting of: ITGB3, ITGA2B, MYL9, LCN2, TREML1, LCN15, CMTM5, PPBP, and PF4. In a further example, the one or more biomarker for sepsis may be selected from the group consisting of: ITGB3, ITGA2B, MYL9, LCN2, and TREML1.

Each of the biomarkers of sepsis may be used alone, or in combination with any of the sepsis biomarkers in the method of the invention. For example, any combination of 1 or more, 2 or more, 3 or more, 4 or more, 5 or more, 6 or more, 7 or more, 8 or more, 9 or more, 10 or more, 11 or more, 12 or more, 13 or more, 14 or more, up to and including all of the sepsis biomarkers may be used to distinguish between sepsis and SIRS in a patient.

For example, the method may be performed using 1 or more biomarker for sepsis selected from the group consisting of: ITGB3, ITGA2B, MYL9, LCN2, and TREML1. For example, the method may be performed using 2 or more biomarkers for sepsis selected from the group consisting of: ITGB3, ITGA2B, MYL9, LCN2, and TREML1. For example, the method may be performed using 3 or more biomarkers for sepsis selected from the group consisting of: ITGB3, ITGA2B, MYL9, LCN2, and TREML1. For example, the method may be performed using 4 or more biomarkers for sepsis selected from the group consisting of: ITGB3, ITGA2B, MYL9, LCN2, and TREML1. For example, the method may be performed using the combination of: ITGB3, ITGA2B, MYL9, LCN2, and TREML1. This combination of sepsis biomarkers was shown to be particularly effective in distinguishing sepsis from SIRS when tested by ROC analysis (see Example 2).

In one embodiment, the one or more biomarker for sepsis is LCN2. In one embodiment, the one or more biomarker for sepsis is ITGA2B. In one embodiment, the one or more biomarker for sepsis is MYL9. In one embodiment, the one or more biomarker for sepsis is ITGB3. In one embodiment, the one or more biomarker for sepsis is TREML1. In one embodiment, the one or more biomarker for sepsis is LCN15. In one embodiment, the one or more biomarker for sepsis is CMTM5. In one embodiment, the one or more biomarker for sepsis is PPBP. In one embodiment, the one or more biomarker for sepsis is PF4. In one embodiment, the one or more biomarker for sepsis is MAP1A. In one embodiment, the one or more biomarker for sepsis is SELP. In one embodiment, the one or more biomarker for sepsis is NEXN. In one embodiment, the one or more biomarker for sepsis is NLRC4. In one embodiment, the one or more biomarker for sepsis is CLEC1B.

As described above for the method for diagnosis of sepsis in a patient, one or more additional biomarker for sepsis may also be used in the distinguishing method. All embodiments described above for the one or more additional biomarker used in the method for diagnosis of sepsis in a patient apply equally to the method for distinguishing between sepsis and SIRS in a patient. For example, if the one or more biomarker is LCN15, the one or more additional biomarker may be selected from at least 1 (eg. at least 2, at least 3, at least 4, at least 5, at least 6, at least 7, at least 8, at least 9, at least 10, at least 11, up to and including all) of the biomarkers: ITGB3, ITGA2B, MYL9, LCN2, TREML1, CMTM5, PPBP, PF4, KIF2C, MAP1A, SELP, NEXN, NLRC4, and CLEC1B. In one embodiment, the one or more additional biomarker is selected from at least 1 (eg. at least 2, at least 3, at least 4, at least 5, at least 6, at least 7, up to and including all) of the biomarkers: ITGB3, ITGA2B, MYL9, LCN2, TREML1, CMTM5, PPBP, and PF4.

In a preferred embodiment, the method for distinguishing between sepsis and SIRS in a patient may comprise:

(i) determining the presence and/or amount of one or more biomarker for sepsis in a sample obtained from a patient, wherein the one or more biomarker for sepsis is selected from the group consisting of: ITGB3, ITGA2B, MYL9, LCN2, and TREML1,
(ii) comparing the presence and/or amount of the one or more biomarker for sepsis determined in said sample in (i) to a corresponding reference value (such as a reference value that is representative of a healthy individual); and thereby determining whether the patient has or is at risk of having sepsis and/or SIRS.

Preferably, the one or more biomarker for sepsis comprises the combination of: ITGB3, ITGA2B, MYL9, LCN2, and TREML1. The patient used in these methods has preferably been diagnosed as having a systemic inflammatory condition (eg. preferably using the method described for diagnosis of a systemic inflammatory condition).

The “one or more biomarker for SIRS” is as defined above for the method for diagnosing SIRS in a patient, and includes any of the one or more SIRS biomarkers described herein (with or without the one or more additional biomarker), and further includes any of the combinations of SIRS biomarkers described herein.

For example, the one or more biomarker for SIRS may be selected from the group consisting of: PLA2G7, ARHGEF10L, MYCL, TGFBI, GPR124, IL1 RN, NLRP3, RBP4, and MPP3. For example, the one or more biomarker for SIRS may be selected from the group consisting of: PLA2G7, ARHGEF10L, MYCL, TGFBI, and GPR124. For example, the one or more biomarker for SIRS may be selected from the group consisting of: PLA2G7, ARHGEF10L, MYCL, and TGFBI.

Each of the biomarkers of SIRS may be used alone, or in combination with any of the SIRS biomarkers in the method of the invention. For example, any combination of 1 or more, 2 or more, 3 or more, 4 or more, 5 or more, 6 or more, 7 or more, 8 or more, up to and including all of the SIRS biomarkers may be used to distinguish between sepsis and SIRS in a patient.

For example, the distinguishing method may be performed using 1 or more biomarker for SIRS selected from the group consisting of: PLA2G7, ARHGEF10L, MYCL, and TGFBI. For example, the distinguishing method may be performed using 2 or more biomarkers for SIRS selected from the group consisting of: PLA2G7, ARHGEF10L, MYCL, and TGFBI. For example, the distinguishing method may be performed using 3 or more biomarkers for SIRS selected from the group consisting of: PLA2G7, ARHGEF10L, MYCL, and TGFBI. For example, the distinguishing method may be performed using all 4 biomarkers for SIRS selected from the group consisting of: PLA2G7, ARHGEF10L, MYCL, and TGFBI. This combination of SIRS biomarkers was shown to be particularly effective in distinguishing sepsis from SIRS when tested by ROC analysis (see Example 2).

In one embodiment, the one or more biomarker for SIRS is TGFBI. In one embodiment, the one or more biomarker for SIRS is PLA2G7. In one embodiment, the one or more biomarker for SIRS is MYCL. In one embodiment, the one or more biomarker for SIRS is ARHGEF10L. In one embodiment, the one or more biomarker for SIRS is GPR124. In one embodiment, the one or more biomarker for SIRS is URN. In one embodiment, the one or more biomarker for SIRS is NLRP3. In one embodiment, the one or more biomarker for SIRS is RBP4. In one embodiment, the one or more biomarker for SIRS is MPP3.

As described above for the method for diagnosis of SIRS in a patient, one or more additional biomarker for SIRS may also be used in the distinguishing method. All embodiments described above for the one or more additional biomarker used in the method for diagnosis of SIRS in a patient apply equally to the method for distinguishing between sepsis and SIRS in a patient. For example, if the one or more biomarker is GPR124, the one or more additional biomarker may be selected from at least 1 (eg. at least 2, at least 3, at least 4, at least 5, at least 6, at least 7, up to and including all) of the biomarkers: PLA2G7, ARHGEF10L, MYCL, TGFBI, URN, NLRP3, RBP4, and MPP3. In one embodiment, the one or more additional biomarker is selected from at least 1 (eg. at least 2, at least 3, up to and including all) of the biomarkers: PLA2G7, ARHGEF10L, MYCL, and TGFBI.

In a preferred embodiment, the method for distinguishing between sepsis and SIRS in a patient may comprise:

(i) determining the presence and/or amount of one or more biomarker for SIRS in a sample obtained from a patient, wherein the one or more biomarker for SIRS is selected from the group consisting of: PLA2G7, ARHGEF10L, MYCL, and TGFBI,
(ii) comparing the presence and/or amount of the one or more biomarker for SIRS determined in said sample in (i) to a corresponding reference value (such as a reference value that is representative of a healthy individual); and thereby determining whether the patient has or is at risk of having sepsis and/or SIRS.

Preferably, the one or more biomarker for SIRS comprises the combination of: PLA2G7, ARHGEF10L, MYCL, and TGFBI. The patient used in this method has preferably been diagnosed as having a systemic inflammatory condition (eg. preferably using the method described for diagnosis of a systemic inflammatory condition).

Any combination of the one or more biomarker for sepsis described herein (including the one or more additional biomarker for sepsis) may be used in conjunction with any combination of the one or more biomarker for SIRS described herein (including the one or more additional biomarker for SIRS) in the method of the invention for distinguishing between sepsis and SIRS in a patient.

For example, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, up to and including all) of the sepsis biomarkers selected from the group consisting of: ITGB3, ITGA2B, MYL9, LCN2, TREML1, LCN15, CMTM5, PPBP, PF4, KIF2C, MAP1A, SELP, NEXN, NLRC4, and CLEC1B, may be used in conjunction with any combination of 1 or more (eg. 2 or more, 3 or more, 4 or more, 5 or more, 6 or more, 7 or more, 8 or more, up to and including all) of the SIRS biomarkers selected from the group consisting of: PLA2G7, ARHGEF10L, MYCL, TGFBI, GPR124, IL1 RN, NLRP3, RBP4, and MPP3, to distinguish between sepsis and SIRS in a patient according to the method described herein.

For example, any combination of 1 or more (eg. 2 or more, or all 3) of the sepsis biomarkers selected from the group consisting of: LCN15, LCN2, and NLRC4, may be used in conjunction with any combination of 1 or more (eg. 2 or more, 3 or more, 4 or more, up to and including all) of the SIRS biomarkers selected from the group consisting of: GPR124, TGFBI, PLA2G7, MYCL, and ARHGEF10L, to distinguish between sepsis and SIRS in a patient according to the method described herein.

For example, any combination of 1 or more (eg. 2 or more, 3 or more, 4 or more, 5 or more, 6 or more, 7 or more, 8 or more, up to and including all) of the sepsis biomarkers selected from the group consisting of: ITGB3, ITGA2B, MYL9, LCN2, TREML1, LCN15, CMTM5, PPBP, and PF4, may be used in conjunction with any combination of 1 or more (eg. 2 or more, 3 or more, 4 or more, up to and including all) of the SIRS biomarkers selected from the group consisting of: PLA2G7, ARHGEF10L, MYCL, TGFBI, and GPR124, to distinguish between sepsis and SIRS in a patient according to the method described herein.

In one embodiment, any combination of 1 or more (eg. 2 or more, 3 or more, 4 or more, up to and including all) of the sepsis biomarkers selected from the group consisting of: ITGB3, ITGA2B, MYL9, LCN2, and TREML1, may be used in conjunction with any combination of 1 or more (eg. 2 or more, 3 or more, up to and including all) of the SIRS biomarkers selected from the group consisting of: PLA2G7, ARHGEF10L, MYCL, and TGFBI, to distinguish between sepsis and SIRS in a patient according to the method described herein.

In one embodiment, any combination of 1 or more (eg. 2 or more, 3 or more, 4 or more, up to and including all) of the sepsis biomarkers selected from the group consisting of: ITGB3, ITGA2B, MYL9, LCN2, and TREML1, may be used in conjunction with any combination of 1 or more (eg. 2 or more, up to and including all) of the SIRS biomarkers selected from the group consisting of: ARHGEF10L, MYCL, and TGFBI, to distinguish between sepsis and SIRS in a patient according to the method described herein.

In one embodiment, the combination of preferred sepsis biomarkers: ITGB3, ITGA2B, MYL9, LCN2, and TREML1, may be used in conjunction with the combination of preferred SIRS biomarkers: PLA2G7, ARHGEF10L, MYCL, and TGFBI, to distinguish between sepsis and SIRS in a patient according to the method described herein.

In one embodiment, the combination of preferred sepsis biomarkers: ITGB3, ITGA2B, MYL9, LCN2, and TREML1, may be used in conjunction with the combination of preferred SIRS biomarkers: ARHGEF10L, MYCL, and TGFBI, to distinguish between sepsis and SIRS in a patient according to the method described herein.

In a further example, the following combinations of sepsis and SIRS biomarkers may be used to distinguish sepsis and SIRS according to the method described herein: (i) LCN15 and TGFBI; (ii) LCN15 and PLA2G7; (iii) LCN15 and GPR124; (iv) LCN15 and MYCL; (v) LCN15 and ARHGEF10L; (vi) ITGA2B and TGFBI; (vii) ITGA2B and PLA2G7; (viii) ITGA2B and GPR124; (ix) ITGA2B and MYCL; (x) ITGA2B and ARHGEF10L; (xi) MYL9 and TGFBI; (xii) MYL9 and PLA2G7; (xiii) MYL9 and GPR124; (xiv) MYL9 and MYCL; (xv) MYL9 and ARHGEF10L; (xvi) CMTM5 and TGFBI; (xvii) CMTM5 and PLA2G7; (xviii) CMTM5 and GPR124; (xix) CMTM5 and MYCL; (xx) CMTM5 and ARHGEF10L; (xxi) PPBP and TGFBI; (xxii) PPBP and PLA2G7; (xxiii) PPBP and GPR124; (xxiv) PPBP and MYCL; (xxv) PPBP and ARHGEF10L; (xxvi) TREML1 and TGFBI; (xxvii) TREML1 and PLA2G7; (xxviii) TREML1 and GPR124; (xxxix) TREML1 and MYCL; (xl) TREML1 and ARHGEF10L; (xli) PF4 and TGFBI; (xlii) PF4 and PLA2G7; (xliii) PF4 and GPR124; (xliv) PF4 and MYCL; and (xlv) PF4 and ARHGEF10L; (xlvi) LCN2 and GPR124; (xlvii) LCN2 and TGFBI; (xlviii) LCN2 and PLA2G7; (xlix) LCN2 and MYCL; (I) LCN2 and ARHGEF10L; (Ii) ITGB3 and GPR124; ITGB3 and TGFBI; ITGB3 and PLA2G7; (liv) ITGB3 and MYCL; and (lv) ITGB3 and ARHGEF10L.

The one or more additional biomarker for sepsis (described herein) and/or the one or more additional biomarker SIRS (described herein) may also be used together with these combinations of biomarkers in the distinguishing method described herein.

In a preferred embodiment, the method for distinguishing between sepsis and SIRS in a patient may comprise:

(i) determining the presence and/or amount of one or more biomarker for sepsis, and one or more biomarker for SIRS in a sample obtained from a patient, wherein the one or more biomarker for sepsis is selected from the group consisting of: ITGB3, ITGA2B, MYL9, LCN2, and TREML1 and the one or more biomarker for SIRS is selected from the group consisting of: PLA2G7, ARHGEF10L, MYCL, and TGFBI,
(ii) comparing the presence and/or amount of the one or more biomarker for sepsis determined in said sample in (i) to a corresponding reference value (such as a reference value that is representative of a healthy individual);
(iii) comparing the presence and/or amount of the one or more biomarker for SIRS in said sample in (i) to a corresponding reference value (such as a reference value that is representative of a healthy individual); and thereby determining whether the patient has sepsis and/or SIRS.

Preferably, the one or more biomarker for sepsis comprises the combination of ITGB3, ITGA2B, MYL9, LCN2, and TREML1 and the one or more biomarker for SIRS comprises the combination of: PLA2G7, ARHGEF10L, MYCL, and TGFB. The patient used in this method has preferably been diagnosed as having a systemic inflammatory condition (eg. preferably using the method described for diagnosis of a systemic inflammatory condition).

All embodiments relating to the step for “determining the presence and/or amount of one or more biomarker in a sample” and for the “comparison” step for making a conclusion about the disease status of the patient as defined above for the “method for diagnosing SIRS” and the “method for diagnosing sepsis” apply equally to the “method for distinguishing between sepsis and SIRS in a patient”. This includes all embodiments relating to the reference value used in these methods.

As described herein, the present inventors observed that the sepsis biomarkers increased in abundance in patients having sepsis as compared to patients having SIRS, as well as healthy individuals. Likewise, the SIRS biomarkers were observed to increase in abundance in patients having SIRS as compared to patients having sepsis, as well as healthy individuals. These differences in marker abundance can be used to determine whether an individual has or is at risk of developing sepsis and/or SIRS.

For example, by comparing the presence and/or amount of markers quantified in a sample obtained from a patient to the presence and/or amount of markers quantified for a reference value (such as a reference value that is representative of a healthy individual (or a population of healthy individuals), a reference value that is representative of an individual having sepsis (or a population of individuals having sepsis), and/or a reference value that is representative of an individual having SIRS (or a population of individuals having SIRS)), it is possible to diagnose the presence (or absence) of sepsis and/or SIRS in a patient. The method permits classification of the individual as belonging to or not belonging to the reference population (ie. by determining whether the amounts of marker quantified in the individual are statistically similar to the reference population or statistically deviate from the reference population). Hence, classification of the individual's marker profile (ie. the overall pattern of change observed for the markers quantified) as corresponding to the profile derived from a particular reference population is predictive that the individual falls (or does not fall) within the reference population.

All embodiments described above (in the context of the methods for diagnosis of sepsis) for the classification of a patient as having or being at risk of having sepsis (or not having or not being at risk of having sepsis) in the method for diagnosis of sepsis in a patient apply equally to the method for distinguishing between sepsis and SIRS in a patient. Likewise, all embodiments described above (in the context of the methods for diagnosis of SIRS) for the classification of a patient as having or being at risk of having SIRS (or not having or not being at risk of having SIRS) in the method for diagnosis of SIRS in a patient apply equally to the method for distinguishing between sepsis and SIRS in a patient. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the “method of diagnosing a systemic inflammatory condition in a patient”, the “method for diagnosing sepsis in a patient” and the “method for diagnosing SIRS in a patient”. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having sepsis (or a population of individuals having sepsis). For example, the reference value may be representative of an individual having abdominal sepsis and/or an individual having pulmonary sepsis (or a population of individuals having abdominal sepsis and/or a population of individuals having pulmonary sepsis).

As described herein, the present inventors observed that the “SIRS” biomarkers described herein each increase in abundance in samples obtained from patients having SIRS, as compared to healthy individuals. Likewise, the “sepsis” biomarkers were also observed to increase in abundance in samples obtained from patients having sepsis, as compared to healthy individuals. Detection of increased levels of the “SIRS” biomarkers in a patient as compared to the levels detected for healthy individuals can thus be used to diagnose the presence of SIRS. Whilst, detection of increased levels of the “sepsis” biomarkers in a patient as compared to the levels detected for healthy individuals can thus be used to diagnose the presence of sepsis. By combining the results from these analyses, a patient can be diagnosed as having sepsis or SIRS.

Thus, in one embodiment, when the reference value is representative of a healthy individual (or a population of healthy individuals), an increase in the one or more biomarker (and/or one or more additional biomarker) for SIRS in the sample obtained from the patient relative to the corresponding reference value representative of a healthy individual, indicates that the patient has or is at risk of developing SIRS. Likewise, no increase or a decrease in the one or more SIRS biomarker (and/or one or more additional biomarker) in the sample obtained from the patient relative to the corresponding reference value representative of a healthy individual, indicates that the patient does not have SIRS. Further confirmation of the diagnosis may be obtained when no increase is observed in the one or more biomarker for sepsis, in the sample obtained from the patient relative to the corresponding reference value representative of a healthy individual. This indicates that the patient has or is at risk of developing SIRS, but does not have sepsis. Furthermore, no increase or a decrease in the one or more SIRS biomarker (and/or one or more additional biomarker) in the sample obtained from the patient relative to the corresponding reference value representative of a healthy individual, may indicate that the patient has or is at risk of having sepsis (e.g. where the patient has already been diagnosed as having a systemic inflammatory condition).

An increase in the one or more biomarker (and/or one or more additional biomarker) for sepsis in the sample obtained from the patient relative to the corresponding reference value representative of a healthy individual, indicates that the patient has or is at risk of developing sepsis. Likewise, no increase or a decrease in the one or more sepsis biomarker (and/or one or more additional biomarker) in the sample obtained from the patient relative to the corresponding reference value representative of a healthy individual, indicates that the patient does not have sepsis. Further confirmation of the diagnosis may be obtained when no increase is observed in the one or more biomarker for SIRS, in the sample obtained from the patient relative to the corresponding reference value representative of a healthy individual. This indicates that the patient has or is at risk of developing sepsis, but does not have SIRS. Furthermore, no increase or a decrease in the one or more sepsis biomarker (and/or one or more additional biomarker) in the sample obtained from the patient relative to the corresponding reference value representative of a healthy individual, may indicate that the patient has or is at risk of having SIRS (e.g. where the patient has already been diagnosed as having a systemic inflammatory condition).

Furthermore, the patient may be diagnosed as having sepsis and SIRS. The patient may be diagnosed as having sepsis and SIRS when an increase is observed in the one or more biomarker for sepsis in the sample obtained from the patient relative to the corresponding reference value representative of a healthy individual; and an increase is observed in the one or more biomarker for SIRS in the sample obtained from the patient relative to the corresponding reference value representative of a healthy individual.

For some of the SIRS biomarkers identified by the present inventors, increased levels of these markers were also observed in patients having sepsis as compared to healthy individuals, although much bigger increases were observed for these biomarkers in the patients having SIRS. Similarly, for some of the sepsis biomarkers identified by the present inventors, increased levels of these markers were also observed in patients having SIRS as compared to healthy individuals, although much bigger increases were observed for these biomarkers in the patients having sepsis. The accuracy of the method for distinguishing between sepsis and SIRS in a patient can thus be improved by looking for a “minimum” fold increase or % increase in the levels of the one or more sepsis biomarker and the one or more SIRS biomarker as compared to the corresponding reference value that is representative of a healthy individual. The fold increase or % increase may be as defined above for the method for diagnosis of a systemic inflammatory condition, the method for diagnosis of sepsis and the method for diagnosis of SIRS.

In one embodiment, the minimum fold increase for the one or more sepsis biomarker (eg. LCNI5, LCN2, ITGA2B, MYL9, ITGB3, CMTM5, PPBP, TREML1, PF4, KIF2C, MAP1A, SELP, NEXN, NLRC4, and CLEC1B) is as defined above for the method for diagnosing sepsis in a patient. For example, for the biomarker LCN15, an increase of at least 1 (eg. at least 1.1, at least 1.2, at least 1.3, at least 1.4, or at least 1.5) fold in LCN15 in the sample obtained from the patient relative to the corresponding reference value, indicates that the patient has, or is at risk of developing sepsis. In one embodiment, no increase or an increase of less than 1 (eg. less than 1.1, less than 1.2, less than 1.3, less than 1.4, less than 1.5) fold in LCN15 in the sample obtained from the patient relative to the corresponding reference value, indicates that the patient does not have, or is not at risk of developing sepsis.

In one embodiment, the minimum fold increase for the one or more SIRS biomarker (eg. GPR124, TGFBI, PLA2G7, MYCL, ARHGEF10L, IL1RN, NLRP3, RBP4, and MPP3) is as defined above for the method for diagnosing SIRS in a patient. For example, for the GRP124 biomarker, an increase of at least 1.1 (eg. at least 1.2, at least 1.3, at least 1.4, at least 1.5) fold in GPR124 in the sample obtained from the patient relative to the corresponding reference value, indicates that the patient has, or is at risk of developing SIRS. In one embodiment, no increase or an increase of less than 1.1 (eg. less than 1.2, less than 1.3, less than 1.4, less than 1.5, less than 1.6) fold in GPR124 in the sample obtained from the patient relative to the corresponding reference value, indicates that the patient does not have, or is not at risk of developing SIRS.

As described herein, the present inventors observed that the levels of the one or more SIRS biomarkers were elevated in patients having SIRS as compared to patients having sepsis. Detection of increased levels of these biomarkers in a patient as compared to the levels detected for patients having sepsis can thus be used to diagnose the presence of SIRS. Thus, in one embodiment, when the reference value is representative of an individual (or population of individuals) having sepsis (such as abdominal sepsis and/or pulmonary sepsis), an increase in the one or more biomarker for SIRS (and/or one or more additional biomarker) in the sample obtained from the patient relative to the corresponding reference value representative of an individual having sepsis, indicates that the patient has or is at risk of developing SIRS. The increase in the one or more SIRS biomarker may be as defined above for the method for diagnosing SIRS described above. Likewise, no increase in the one or more biomarker (and/or one or more additional biomarker) in the sample obtained from the patient relative to the corresponding reference value representative of an individual having sepsis, indicates that the patient does not have SIRS.

As described herein, the present inventors observed that the levels of the one or more sepsis biomarkers were elevated in patients having sepsis as compared to patients having SIRS. Detection of increased levels of these biomarkers in a patient as compared to the levels detected for patients having SIRS can thus be used to diagnose the presence of sepsis. Thus, in one embodiment, when the reference value is representative of an individual (or population of individuals) having SIRS, an increase in the one or more sepsis biomarker (and/or one or more additional biomarker) in the sample obtained from the patient relative to the corresponding reference value representative of an individual having SIRS, indicates that the patient has or is at risk of developing sepsis (such as abdominal sepsis and/or pulmonary sepsis). The increase in the one or more sepsis biomarker may be as defined above for the method for diagnosing sepsis described above. Likewise, no increase in the one or more biomarker (and/or one or more additional biomarker) in the sample obtained from the patient relative to the corresponding reference value representative of an individual having SIRS, indicates that the patient does not have sepsis.

As described above for the “method for diagnosing a systemic inflammatory condition in a patient”, the method of the invention may involve the use of multiple separate reference values. All combinations of reference values defined above for the “method for diagnosing a systemic inflammatory condition in a patient” apply equally to the method for distinguishing between sepsis and SIRS.

For example, the reference value used in the method may comprise: (i) a reference value that is representative of an individual (or population of individuals) having sepsis and a separate reference value that is representative of an individual (or population of individuals) having SIRS. In one embodiment, the patient may be diagnosed as having sepsis and SIRS, when an increase is observed in the one or more biomarker for sepsis in the sample obtained from the patient relative to the corresponding reference value representative of an individual having SIRS; and an increase is observed in the one or more biomarker for SIRS in the sample obtained from the patient relative to the corresponding reference value representative of an individual having sepsis.

The method for distinguishing between sepsis and SIRS in a patient as described herein can be used in a decision tree process to investigate the health of a patient having or suspected of having a systemic inflammatory condition. For example, the method for distinguishing sepsis and SIRS in a patient can be performed before, after, or in addition to any of the other methods of the invention described herein.

In one embodiment, the method for distinguishing sepsis and SIRS in a patient is performed as described herein. If the patient tests positive for sepsis, the patient may be further tested for sepsis, abdominal sepsis and/or pulmonary sepsis using the diagnostic methods described herein, so as to confirm whether the patient has or is at risk of developing sepsis, and/or determine whether the patient has or is at risk of developing abdominal sepsis and/or pulmonary sepsis. If the patient tests positive for SIRS, the patient may be further tested for SIRS using the diagnostic method described herein, so as to confirm whether the patient has or is at risk of developing SIRS.

In one embodiment, the method for distinguishing sepsis and SIRS in a patient (as described herein) can be performed subsequent to (or in addition to) the method for diagnosing whether a patient has a systemic inflammatory condition (as described herein). For example, if the patient tests positive for a systemic inflammatory condition (using the method for diagnosing whether a patient has a systemic inflammatory condition), they may be tested using the distinguishing method described herein to determine whether they have sepsis and/or SIRS. In one embodiment, the above combination of methods are performed as described, and if the patient tests positive for sepsis, the patient may be further tested for sepsis, abdominal sepsis and/or pulmonary sepsis using the diagnostic methods described herein, so as to confirm whether the patient has or is at risk of developing sepsis, and/or to determine whether the patient has or is at risk of developing abdominal sepsis and/or pulmonary sepsis. In one embodiment, the above combination of methods are performed as described, and if the patient tests positive for SIRS, the patient may be further tested for SIRS using the diagnostic method described herein, so as to confirm whether the patient has or is at risk of developing SIRS.

The above described combination of methods may also be performed in parallel to determine the disease status of a patient by simultaneously (or substantially simultaneously) investigating the expression of all the biomarkers in a sample obtained from the patient, and determining whether the patient has or is at risk of having a systemic inflammatory condition, sepsis (such as abdominal or pulmonary sepsis) and/or SIRS.

When performing these different methods in a decision tree process, the sample used in each step of the method may be the same sample obtained from the patient (as described herein). When the method comprises multiple quantification steps, these multiple steps may be performed at the same time (e.g. in parallel) and/or using the same sample. When the method comprises multiple comparison steps, these multiple steps may be performed at the same time (e.g. in parallel).

For example, the method for distinguishing between sepsis and SIRS in a patient may comprise:

(a) diagnosing a patient as having a systemic inflammatory condition by performing a method comprising:
(i) determining the presence and/or amount of one or more inflammatory biomarker in a sample obtained from the patient, wherein the one or more inflammatory biomarker is selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1;
(ii) comparing the presence and/or amount of the one or more inflammatory biomarker determined in said sample in (i) to a corresponding reference value (such as a value that is representative of a healthy individual); and thereby determining that the patient has a systemic inflammatory condition;
   (b) determining whether the patient diagnosed as having a systemic inflammatory condition has sepsis and/or SIRS by performing a method comprising:
(i) determining the presence and/or amount of one or more biomarker for sepsis, and/or one or more biomarker for SIRS in a sample obtained from a patient, wherein the one or more biomarker for sepsis is selected from the group consisting of: ITGB3, ITGA2B, MYL9, LCN2, TREML1, LCN15, CMTM5, PPBP, and PF4; and the one or more biomarker for SIRS is selected from the group consisting of: PLA2G7, ARHGEF10L, MYCL, TGFBI, and GPR124;
(ii) comparing the presence and/or amount of the one or more biomarker for sepsis determined in said sample in (i) to a corresponding reference value (such as a value that is representative of a healthy individual) and/or comparing the presence and/or amount of the one or more biomarker for SIRS in said sample in (i) to a corresponding reference value (such as a value that is representative of a healthy individual); and thereby determining whether the patient has sepsis and/or SIRS.

In a further example, a method may be performed to distinguish between sepsis and SIRS in a patient, comprising;

(a) diagnosing a patient as having a systemic inflammatory condition by performing a method comprising:
(i) determining the presence and/or amount of one or more inflammatory biomarker in a sample obtained from the patient, wherein the one or more inflammatory biomarker is selected from the group consisting of: FAM20A, OLAH, and CD177;
(ii) comparing the presence and/or amount of the one or more inflammatory biomarker determined in said sample in (i) to a corresponding reference value (such as a value that is representative of a healthy individual); and thereby determining that the patient has a systemic inflammatory condition;
   (b) determining whether the patient diagnosed as having a systemic inflammatory condition has sepsis and/or SIRS by performing a method comprising:
(i) determining the presence and/or amount of one or more biomarker for sepsis and/or one or more biomarker for SIRS in a sample obtained from a patient, wherein the one or biomarker for sepsis is selected from the group consisting of: ITGA2B, ITGB3, MYL9, LCN2, and TREML1, and the one or more biomarker for SIRS is selected from the group consisting of: PLA2G7, ARHGEF10L, MYCL, and TGFBI;
(ii) comparing the presence and/or amount of the one or more biomarker for sepsis determined in said sample in (i) to a corresponding reference value (such as a value that is representative of a healthy individual) and/or comparing the presence and/or amount of the one or more biomarker for SIRS in said sample in (i) to a corresponding reference value (such as a value that is representative of a healthy individual); and thereby determining whether the patient has sepsis and/or SIRS.

In a related aspect, the present invention also provides the use of one or more biomarker for sepsis (as described herein), and/or one or more biomarker for SIRS (as described herein) for distinguishing between sepsis and SIRS in a patient.

In one embodiment, the invention provides the use of one or more biomarker for sepsis selected from the group consisting of: ITGA2B, ITGB3, MYL9, LCN2, TREML1, LCN15, CMTM5, PPBP, PF4, KIF2C, MAP1A, SELP, NEXN, NLRC4, and CLEC1B, and/or one or more biomarker for SIRS selected from the group consisting of: PLA2G7, ARHGEF10L, MYCL, TGFBI, GPR124, IL1 RN, NLRP3, RBP4, and MPP3, for distinguishing between sepsis and SIRS in a patient. In one embodiment, the invention provides the use of one or more biomarker for sepsis selected from the group consisting of: ITGA2B, ITGB3, MYL9, LCN2, TREML1, LCN15, CMTM5, PPBP, and PF4, and/or one or more biomarker for SIRS selected from the group consisting of: PLA2G7, ARHGEF10L, MYCL, TGFBI, and GPR124, for distinguishing between sepsis and SIRS in a patient. In one embodiment, the invention provides the use of one or more biomarker for sepsis selected from the group consisting of: ITGA2B, ITGB3, MYL9, LCN2, and TREML1, and/or one or more biomarker for SIRS selected from the group consisting of: PLA2G7, ARHGEF10L, MYCL and TGFBI, for distinguishing between sepsis and SIRS in a patient. In one embodiment, the invention provides the use of the sepsis biomarkers: ITGA2B, ITGB3, MYL9, LCN2, and TREML1, and/or the SIRS biomarkers PLA2G7, ARHGEF10L, MYCL, and TGFBI, for distinguishing between sepsis and SIRS in a patient. In one embodiment, the invention provides the use of the sepsis biomarkers: ITGA2B, ITGB3, MYL9, LCN2, and TREML1, and/or the SIRS biomarkers ARHGEF10L, MYCL, and TGFBI, for distinguishing between sepsis and SIRS in a patient.

All embodiments described above for the method of distinguishing between sepsis and SIRS in a patient apply equally to the ‘use’ of the invention described herein. This includes all embodiments relating to the terms “sepsis”, “SIRS”, “patient”, “sample”, “the one or more biomarker for sepsis”, and “the one or more biomarker for SIRS” (including all combinations of sepsis and SIRS biomarkers described above).

At present, there is no clinical test available for distinguishing between abdominal sepsis and pulmonary sepsis. Rapid diagnosis of the physiological origin of sepsis in a patient would however be useful for selecting the most appropriate treatment for patients having sepsis. The present inventors have identified a set of biomarkers that is predictive of abdominal sepsis and a separate set of biomarkers that is predictive of pulmonary sepsis in patients. Using these distinct sets of biomarkers, the present inventors have developed a rapid and sensitive way to distinguish between abdominal sepsis and pulmonary sepsis in a patient by simultaneously quantifying one or more biomarker for abdominal sepsis and/or one or more biomarker for pulmonary sepsis in a sample obtained from a patient, so as to determine whether the patient has a biomarker profile that is predictive of abdominal or pulmonary sepsis.

The present invention therefore provides a method for distinguishing between abdominal sepsis and pulmonary sepsis in a patient, comprising:

(i) determining the presence and/or amount of one or more biomarker for abdominal sepsis (as described herein), and/or one or more biomarker for pulmonary sepsis (as described herein) in a sample obtained from a patient,
(ii) comparing the presence and/or amount of the one or more biomarker for abdominal sepsis and/or the one or more for pulmonary sepsis determined in said sample in (i) to a corresponding reference value;
and thereby determining whether the patient has abdominal sepsis and/or pulmonary sepsis.

As illustrated in Example 2, the distinguishing method of the invention can be performed using only one or more of the abdominal sepsis biomarker described herein, or only one or more of the pulmonary sepsis biomarkers described herein. These biomarkers can be used on their own to distinguish abdominal and pulmonary sepsis because their expression correlates with the patient's disease condition (i.e. the presence and/or amount of these biomarkers depends on whether a patient has abdominal sepsis or pulmonary sepsis or is healthy). Determining the presence and/or amount of either of these biomarkers and comparing this to a corresponding reference value (such as a reference value that is representative of a healthy individual, an abdominal sepsis patient and/or a pulmonary sepsis patient) therefore allows the disease status of the patient to be determined.

In one embodiment, the present invention therefore provides a method for distinguishing between abdominal sepsis and pulmonary sepsis in a patient, comprising:

(i) determining the presence and/or amount of one or more biomarker for abdominal sepsis (as described herein) in a sample obtained from a patient,
(ii) comparing the presence and/or amount of the one or more biomarker for abdominal sepsis determined in said sample in (i) to a corresponding reference value; and thereby determining whether the patient has abdominal sepsis and/or pulmonary sepsis.

In one embodiment, the present invention therefore provides a method for distinguishing between abdominal sepsis and pulmonary sepsis in a patient, comprising:

(i) determining the presence and/or amount of one or more biomarker for pulmonary sepsis (as described herein) in a sample obtained from a patient,
(ii) comparing the presence and/or amount of the one or more biomarker for pulmonary sepsis determined in said sample in (i) to a corresponding reference value; and thereby determining whether the patient has abdominal sepsis and/or pulmonary sepsis.

Alternatively, the abdominal and pulmonary sepsis biomarkers can be used in combination to distinguish between abdominal sepsis and pulmonary sepsis.

In one embodiment, the present invention provides a method for distinguishing between abdominal sepsis and pulmonary sepsis in a patient, comprising:

(i) determining the presence and/or amount of one or more biomarker for abdominal sepsis (as described herein), and one or more biomarker for pulmonary sepsis (as described herein) in a sample obtained from a patient,
(ii) comparing the presence and/or amount of the one or more biomarker for abdominal sepsis determined in said sample in (i) to a corresponding reference value;
(iii) comparing the presence and/or amount of the one or more biomarker for pulmonary sepsis in said sample in (i) to a corresponding reference value; and thereby determining whether the patient has abdominal sepsis and/or pulmonary sepsis.

All embodiments described above for the “method for diagnosing a systemic inflammatory condition in a patient”, the “method for diagnosing abdominal sepsis” and the “method for diagnosing pulmonary sepsis” apply equally to the “method for distinguishing between abdominal sepsis and pulmonary sepsis in a patient”. This includes all embodiments relating to the “sample”, “patient”, “biomarker”, and “reference value”, and all embodiments relating to the step for “determining the presence and/or amount of one or more biomarker in a sample” and for the “comparison” step for making a conclusion as to the disease status of the patient.

As used herein, “distinguishing between abdominal sepsis and pulmonary sepsis” means to determine whether a patient has or is at risk of developing abdominal sepsis and/or pulmonary sepsis. For example, it may involve determining whether a patient has or is at risk of developing abdominal sepsis or pulmonary sepsis. For example, it may involve determining whether a patient has or is at risk of developing abdominal sepsis and pulmonary sepsis. This may involve distinguishing between a group (ie. one or more) of patients having abdominal sepsis and a group (ie. one or more) of patients having pulmonary sepsis. In one embodiment, this may involve diagnosing or determining whether a patient has or is at risk of developing one or more systemic inflammatory condition selected from: abdominal sepsis and pulmonary sepsis.

The systemic inflammatory conditions “abdominal sepsis” and “pulmonary sepsis” are as described above for the “method for diagnosing a systemic inflammatory condition in a patient”.

The “patient” for which diagnosis is performed is as described above for the “method for diagnosing a systemic inflammatory condition in a patient”. In one embodiment, the patient is suspected of having or being at risk of developing a systemic inflammatory condition. In one embodiment, the patient has been diagnosed as having a systemic inflammatory condition. In one embodiment, the patient has been diagnosed as having a systemic inflammatory condition using the method described herein. In one embodiment, the patient is suspected of having or being at risk of developing sepsis. In one embodiment, the patient has been diagnosed as having sepsis (eg. using the methods described herein for diagnosis of sepsis, or for distinguishing between sepsis and SIRS). In one embodiment, the patient is suspected of having or being at risk of developing abdominal sepsis and/or pulmonary sepsis. In one embodiment, the patient is suspected of having or being at risk of developing abdominal sepsis. In one embodiment, the patient is suspected of having or being at risk of developing pulmonary sepsis.

The “sample” obtained from the patient is as described above for the “method for diagnosing a systemic inflammatory condition in a patient”, the “method for diagnosing abdominal sepsis” and the “method for diagnosing pulmonary sepsis”, including all embodiments relating to the time point at which the sample is obtained.

The “one or more biomarker” of the invention is as described above for the “method for diagnosing a systemic inflammatory condition in a patient”. In one embodiment, the “one or more biomarker” is a nucleic acid, as defined herein. In one embodiment, the “one or more biomarker” is a protein, as defined herein.

The “one or more biomarker for abdominal sepsis” is as described above for the method for diagnosis of abdominal sepsis in a patient, and includes any of the one or more abdominal sepsis biomarkers described herein (with or without the one or more additional biomarker) and further includes any of the combinations of abdominal sepsis biomarkers described herein.

For example, the one or more biomarker for abdominal sepsis may be selected from the group consisting of: SLC39A8, CIQC, CIQA, MRAS, TMEM37, CIQB, PCOLCE2, KIF2C, TNF, IF144, IFIT1, and RPGRIP1. For example, the one or more biomarker for abdominal sepsis may be selected from the group consisting of: SLC39A8, CIQC, CIQA, MRAS, TMEM37, CIQB, PCOLCE2, KIF2C, and TNF. For example, the one or more biomarker for abdominal sepsis may be selected from the group consisting of: SLC39A8, CIQC, CIQA, MRAS, TMEM37, and CIQB. For example, the one or more biomarker for abdominal sepsis may be selected from the group consisting of: SLC39A8, CIQC, and CIQA.

Each of the biomarkers of abdominal sepsis may be used alone, or in combination with any of the abdominal sepsis biomarkers in the method of the invention. For example, any combination of 1 or more, 2 or more, 3 or more, 4 or more, 5 or more, 6 or more, 7 or more, 8 or more, 9 or more, 10 or more, 11 or more, or up to and including all of the abdominal sepsis biomarkers may be used to diagnose abdominal sepsis in a patient according to the method of the invention.

In one embodiment, any combination of 1 or more (eg. 2 or more, 3 or more, 4 or more, 5 or more, or all 6) of the biomarkers selected from the group consisting of: SLC39A8, CIQC, CIQA, MRAS, TMEM37, and CIQB, may be used as a biomarker for abdominal sepsis in the distinguishing method. For example, the combination of SLC39A8, CIQC, CIQA, MRAS, TMEM37, and CIQB may be used. In one embodiment, any combination of 1 or more (eg. 2 or more, or all 3) of the biomarkers selected from the group consisting of: SLC39A8, CIQC, and CIQA may be used as a biomarker for abdominal sepsis in the distinguishing method. For example, the combination of SLC39A8, CIQC, and CIQA may be used.

As described above for the method for diagnosis of abdominal sepsis in a patient, one or more additional biomarker for abdominal sepsis may also be used in the distinguishing method for determining the presence (or absence) of abdominal sepsis in a patient. All embodiments described above for the one or more additional biomarker used in the method for diagnosis of abdominal sepsis in a patient apply equally to the method for distinguishing between abdominal sepsis and pulmonary sepsis in a patient.

In a preferred embodiment, the method for distinguishing between abdominal sepsis and pulmonary sepsis in a patient, may comprise:

(i) determining the presence and/or amount of one or more biomarker for abdominal sepsis in a sample obtained from a patient, wherein the one or more biomarker for abdominal sepsis is selected from the group consisting of: SLC39A8, CIQC, CIQA, TMEM37, and CIQB;
(ii) comparing the presence and/or amount of the one or more biomarker for abdominal sepsis determined in said sample in (i) to a corresponding reference value; and thereby determining whether the patient has abdominal sepsis and/or pulmonary sepsis.

The “one or more biomarker for pulmonary sepsis” is as described above for the method for diagnosis of pulmonary sepsis in a patient, and includes any of the one or more pulmonary sepsis biomarkers described herein (with or without the one or more additional biomarker), and further includes any of the combinations of pulmonary sepsis biomarkers described herein.

For example, the one or more biomarker for pulmonary sepsis may be selected from the group consisting of: HCAR2, CXCR1, DISC1, EPSTI1, and IF144. For example, the one or more biomarker for pulmonary sepsis may be selected from the group consisting of: HCAR2, CXCR1, and DISC1. For example, the one or more biomarker for pulmonary sepsis may be selected from the group consisting of: HCAR2 and CXCR1.

Each of the biomarkers of pulmonary sepsis may be used alone, or in combination with any of the pulmonary sepsis biomarkers in the method of the invention. For example, any combination of 1 or more, 2 or more, 3 or more, 4 or more, or up to and including all of the pulmonary sepsis biomarkers may be used to diagnose pulmonary sepsis in a patient according to the method of the invention.

In one embodiment, any combination of 1 or more (eg. 2 or more, or all 3) of the biomarkers selected from the group consisting of: HCAR2, CXCR1, and DISC1, may be used as a biomarker for pulmonary sepsis in the distinguishing method. For example, the combination of HCAR2, CXCR1, and DISC1 may be used. In one embodiment, HCAR2 and/or CXCR1 may be used as a biomarker for pulmonary sepsis in the distinguishing method.

As described above for the method for diagnosis of pulmonary sepsis in a patient, one or more additional biomarker for pulmonary sepsis may also be used in the distinguishing method for determining the presence (or absence) of pulmonary sepsis in a patient. All embodiments described above for the one or more additional biomarker used in the method for diagnosis of pulmonary sepsis in a patient apply equally to the method for distinguishing between abdominal sepsis and pulmonary sepsis in a patient.

In a preferred embodiment, the method for distinguishing between abdominal sepsis and pulmonary sepsis in a patient, may comprise:

(i) determining the presence and/or amount of one or more biomarker for pulmonary sepsis in a sample obtained from a patient, wherein the one or more biomarker for pulmonary sepsis is selected from the group consisting of: HCAR2, CXCR1, and DISC1,
(ii) comparing the presence and/or amount of the one or more biomarker for pulmonary sepsis determined in said sample in (i) to a corresponding reference value; and thereby determining whether the patient has abdominal sepsis and/or pulmonary sepsis.

As illustrated in Example 2, effective results for distinguishing between abdominal and pulmonary sepsis can be achieved by using the abdominal sepsis in conjunction with the pulmonary sepsis biomarkers. Any combination of the one or more biomarker for abdominal sepsis described herein (including the one or more additional biomarker for abdominal sepsis) may be used in conjunction with any combination of the one or more biomarker for pulmonary sepsis described herein (including the one or more additional biomarker for pulmonary sepsis) in the method for distinguishing between abdominal sepsis and pulmonary sepsis in a patient.

In one embodiment, any combination of 1 or more (eg. 2 or more, 3 or more, 4 or more, 5 or more, 6 or more, 7 or more, 8 or more, 9 or more, 10 or more, 11 or more up to and including all) of the abdominal sepsis biomarkers selected from the group consisting of: SLC39A8, CIQC, CIQA, MRAS, TMEM37, CIQB, PCOLCE2, KIF2C, TNF, IF144, IFIT1, and RPGRIP1, may be used in conjunction with any combination of 1 or more (eg. 2 or more, 3 or more, 4 or more, up to and including all) of the pulmonary sepsis biomarkers selected from the group consisting of: HCAR2, CXCR1, DISC1, EPSTI1, and IF144, to distinguish between sepsis and SIRS in a patient according to the method described herein.

In one embodiment, any combination of 1 or more (eg. 2 or more, 3 or more, 4 or more, 5 or more, 6 or more, 7 or more, 8 or more, 9 or more, 10 or more, 11 or more up to and including all) of the abdominal sepsis biomarkers selected from the group consisting of: SLC39A8, CIQC, CIQA, MRAS, TMEM37, CIQB, PCOLCE2, and KIF2C, may be used in conjunction with any combination of 1 or more (eg. 2 or more, 3 or more, 4 or more, up to and including all) of the pulmonary sepsis biomarkers selected from the group consisting of: HCAR2, CXCR1, DISC1, EPSTI1, and IF144, to distinguish between sepsis and SIRS in a patient according to the method described herein.

In one embodiment, any combination of 1 or more (eg. 2 or more, 3 or more, 4 or more, 5 or more, up to and including all) of the abdominal sepsis biomarkers selected from the group consisting of: SLC39A8, CIQC, CIQA, MRAS, TMEM37, and CIQB, may be used in conjunction with any combination of 1 or more (eg. 2 or more, up to and including all) of the pulmonary sepsis biomarkers selected from the group consisting of: HCAR2, CXCR1, and DISC1, to distinguish between sepsis and SIRS in a patient according to the method described herein. For example, the combination of abdominal sepsis biomarkers SLC39A8, CIQC, CIQA, MRAS, TMEM37, and CIQB, may be used in conjunction with the combination of pulmonary sepsis biomarkers HCAR2, CXCR1, and DISC1, to distinguish between sepsis and SIRS in a patient according to the method described herein.

In one embodiment, any combination of 1 or more (eg. 2 or more, up to and including all) of the abdominal sepsis biomarkers selected from the group consisting of: SLC39A8, CIQC, and CIQA, may be used in conjunction with the pulmonary sepsis biomarkers HCAR2 and/or CXCR1, to distinguish between sepsis and SIRS in a patient according to the method described herein. For example, the combination of the abdominal sepsis biomarkers SLC39A8, CIQC, and CIQA may be used in conjunction with the combination of the pulmonary sepsis biomarkers HCAR2, CXCR1, and DISC1, to distinguish between sepsis and SIRS in a patient according to the method described herein. For example, the combination of abdominal sepsis biomarkers SLC39A8, CIQC, and CIQA may be used in conjunction with the combination of pulmonary sepsis biomarkers HCAR2 and CXCR1 to distinguish between sepsis and SIRS in a patient according to the method described herein.

For example, the following combinations of abdominal sepsis and pulmonary sepsis biomarkers may be used to distinguish between abdominal sepsis and pulmonary sepsis according to the method described herein: (i) MRAS and EPSTI1; (ii) MRAS and DISC1; (iii) MRAS and CXCR1; (iv) MRAS and HCAR2; (v) MRAS and IF144; (vi) PCOLCE2 and EPSTI1; (vii) PCOLCE2 and DISC1; (viii) PCOLCE2 and CXCR1; (ix) PCOLCE2 and HCAR2; (x) PCOLCE2 and IF144; (xi) (xii) TMEM37 and EPSTI1; (xiv) TMEM37 and DISC1; (xv) TMEM37 and CXCR1; (xvi) TMEM37 and HCAR2; (xvii) TMEM37 and IF144; (xviii) SLC39A8 and EPSTI1; (xix) SLC39A8 and DISC1; (xx) SLC39A8 and CXCR1; (xxi) SLC39A8 and HCAR2; (xxii) SLC39A8 and IF144; (xxiii) KIF2C and EPSTI1; (xxiv) KIF2C and DISC1; (xxv) KIF2C and CXCR1; (xxvi) KIF2C and HCAR2; (xxvii) KIF2C and IF144; (xxviii) CIQC and EPSTI1; (xxxix) CIQC and DISC1; (xl) CIQC and CXCR1; (xli) CIQC and HCAR2; (xlii) CIQC and IF144; (xliii) CIQB and EPSTI1; (xliv) CIQB and DISC1; (xlivi) CIQB and CXCR1; (xlivii) CIQB and HCAR2; (xliviii) CIQB and IF144; (xlix) CIQA and EPSTI1; (I) CIQA and DISC1; (Ii) CIQA and CXCR1; CIQA and HCAR2; (liii) CIQA and IF144; (liv) TNF and EPSTI1; (lv) TNF and DISC1; (lvi) TNF and CXCR1; (lvH) TNF and HCAR2; (MO TNF and IF144.

In one embodiment, the biomarker IF144 may be used to distinguish between abdominal sepsis and pulmonary sepsis in a patient (such as patient that has been diagnosed as having sepsis).

The one or more additional biomarker for abdominal sepsis (described herein) and/or the one or more additional biomarker pulmonary sepsis (described herein) may also be used together with these combinations of biomarkers in the distinguishing method described herein.

In a preferred embodiment, the method for distinguishing between abdominal sepsis and pulmonary sepsis in a patient, may comprise:

(i) determining the presence and/or amount of one or more biomarker for abdominal sepsis, and one or more biomarker for pulmonary sepsis in a sample obtained from a patient, wherein the one or more biomarker for abdominal sepsis is selected from the group consisting of: SLC39A8, CIQC, CIQA, TMEM37, and CIQB, and the one or more biomarker for pulmonary sepsis is selected from the group consisting of: HCAR2, CXCR1, and DISC1,
(ii) comparing the presence and/or amount of the one or more biomarker for abdominal sepsis determined in said sample in (i) to a corresponding reference value;
(iii) comparing the presence and/or amount of the one or more biomarker for pulmonary sepsis in said sample in (i) to a corresponding reference value; and thereby determining whether the patient has abdominal sepsis and/or pulmonary sepsis.

All embodiments relating to the step for “determining the presence and/or amount of one or more biomarker in a sample” and for the “comparison” step as described above for the “method for diagnosing abdominal sepsis” and the “method for diagnosing pulmonary sepsis” apply equally to the “method for distinguishing between abdominal sepsis and pulmonary sepsis in a patient”. This includes all embodiments relating to the reference value used in these methods.

As described herein, the present inventors observed that some of the “abdominal sepsis” biomarkers (MRAS, PCOLCE2, TMEM37, SLC39A8, KIF2C, CIQC, CIQB, CIQA, TNF) described herein each increase in abundance in samples obtained from patients having abdominal sepsis, as compared to patients having pulmonary sepsis and/or healthy individuals, whilst others (IF144, IFIT1, and RPGRIP1) decreased in abundance in samples obtained from patients having abdominal sepsis, as compared to patients having pulmonary sepsis and/or healthy individuals. Likewise, the “pulmonary sepsis” biomarkers described herein were also observed to increase in abundance in samples obtained from patients having pulmonary sepsis, as compared to patients having abdominal sepsis and/or healthy individuals. These differences in marker abundance can be used to determine whether an individual has or is at risk of developing abdominal sepsis and/or pulmonary sepsis.

For example, by comparing the amount of markers quantified in a sample obtained from a patient to the amount of markers quantified for a reference value (such as a reference value that is representative of a healthy individual (or a population of healthy individuals), a reference value that is representative of an individual having abdominal sepsis (or a population of individuals having abdominal sepsis), a reference value that is representative of an individual having pulmonary sepsis (or a population of individuals having pulmonary sepsis), and/or a reference value that is representative of an individual having SIRS (or a population of individuals having SIRS)), it is possible to diagnose the presence (or absence) of abdominal sepsis and/or pulmonary sepsis in a patient. The method permits classification of the individual as belonging to or not belonging to the reference population (ie. by determining whether the amounts of marker quantified in the individual are statistically similar to the reference population or statistically deviate from the reference population). Hence, classification of the individual's marker profile (ie. the overall pattern of change observed for the markers quantified) as corresponding to the profile derived from a particular reference population is predictive that the individual falls (or does not fall) within the reference population.

All embodiments described above (in the context of the method for diagnosis of abdominal sepsis) for the classification of a patient as having or being at risk of having (or not having or not being at risk of having) abdominal sepsis in the context of the method for diagnosis of abdominal sepsis apply equally to the method for distinguishing between abdominal sepsis and pulmonary sepsis. All embodiments described above (in the context of the method for diagnosis of pulmonary sepsis) for the classification as a patient as having or being at risk of having (or not having or not being at risk of having) pulmonary sepsis in the context of the method for diagnosis of pulmonary sepsis apply equally to the method for distinguishing between abdominal sepsis and pulmonary sepsis. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the “method of diagnosing a systemic inflammatory condition in a patient”, the “method for diagnosing abdominal sepsis”, and the “method for diagnosing pulmonary sepsis”. In one embodiment, the reference value is representative of a healthy individual (or a population of healthy individuals). In one embodiment, the reference value is representative of an individual having SIRS (or a population of individuals having SIRS). In one embodiment, the reference value is representative of an individual having abdominal sepsis (or a population of individuals having abdominal sepsis). In one embodiment, the reference value is representative of an individual having pulmonary sepsis (or a population of individuals having pulmonary sepsis).

As described herein, the present inventors observed that the “abdominal sepsis” biomarkers SLC39A8, CIQC, CIQA, MRAS, TMEM37, CIQB, PCOLCE2, KIF2C, TNF each increase in abundance in samples obtained from patients having abdominal sepsis, as compared to healthy individuals. Thus, in one embodiment, when the reference value is representative of a healthy individual (or a population of healthy individuals), an increase in the one or more biomarker (and/or one or more additional biomarker) for abdominal sepsis in the sample obtained from the patient relative to the corresponding reference value representative of a healthy individual, indicates that the patient has or is at risk of developing abdominal sepsis. Likewise, no increase in the one or more abdominal sepsis biomarker (and/or one or more additional biomarker) in the sample obtained from the patient relative to the corresponding reference value representative of a healthy individual, indicates that the patient does not have abdominal sepsis.

The inventors also observed a decrease in the abdominal sepsis biomarkers IF144, IFIT1, and RPGRIP1 in samples obtained from patients having abdominal sepsis, as compared to healthy individuals. Thus, in one embodiment, when the reference value is representative of a healthy individual (or a population of healthy individuals), a decrease in the one or more biomarker (and/or one or more additional biomarker) for abdominal sepsis in the sample obtained from the patient relative to the corresponding reference value representative of a healthy individual, indicates that the patient has or is at risk of developing abdominal sepsis. Likewise, no decrease in the one or more abdominal sepsis biomarker (and/or one or more additional biomarker) in the sample obtained from the patient relative to the corresponding reference value representative of a healthy individual, indicates that the patient does not have abdominal sepsis.

Further confirmation of the diagnosis may be obtained when no increase is observed in the one or more biomarker for pulmonary sepsis, in the sample obtained from the patient relative to the corresponding reference value representative of a healthy individual. This indicates that the patient has or is at risk of developing abdominal sepsis, but does not have pulmonary sepsis.

As described above, the inventors observed an increase in the “pulmonary sepsis” biomarkers (HCAR2, CXCR1, DISC1, EPSTI1, and IF144) in samples obtained from patients having pulmonary sepsis, as compared to healthy individuals. Thus, in one embodiment, when the reference value is representative of a healthy individual (or a population of healthy individuals), an increase in the one or more biomarker (and/or one or more additional biomarker) for pulmonary sepsis in the sample obtained from the patient relative to the corresponding reference value representative of a healthy individual, indicates that the patient has or is at risk of developing pulmonary sepsis. Likewise, no increase in the one or more pulmonary sepsis biomarker (and/or one or more additional biomarker) in the sample obtained from the patient relative to the corresponding reference value representative of a healthy individual, indicates that the patient does not have pulmonary sepsis.

Further confirmation of the diagnosis may be obtained when no increase (eg. in any one or more of MRAS, PCOLCE2, TMEM37, SLC39A8, KIF2C, CIQC, CIQB, CIQA, TNF) and/or no decrease (eg. in any one or more of IF144, IFIT1, and RPGRIP1) is observed in the one or more biomarker for abdominal sepsis, in the sample obtained from the patient relative to the corresponding reference value representative of a healthy individual. This indicates that the patient has or is at risk of developing pulmonary sepsis, but does not have abdominal sepsis.

Furthermore, the patient may be diagnosed as having abdominal sepsis and pulmonary sepsis when an increase is observed in any one or more of: SLC39A8, CIQC, CIQA, MRAS, TMEM37, CIQB, PCOLCE2, KIF2C, and TNF, and/or a decrease is observed in any one or more of: IF144, IFIT1, and RPGRIP1, in the sample obtained from the patient relative to the corresponding reference value representative of a healthy individual; and an increase is observed in the one or more biomarker for pulmonary sepsis in the sample obtained from the patient relative to the corresponding reference value representative of a healthy individual.

The accuracy of the method for distinguishing between abdominal sepsis and pulmonary sepsis in a patient can be improved by looking for a “minimum” fold change in the levels of the one or more abdominal sepsis biomarker and the one or more pulmonary sepsis biomarker as compared to the corresponding reference value that is representative of a healthy individual. In one embodiment, the minimum fold change or % change for the abdominal sepsis biomarkers is as defined above for the method for diagnosing abdominal sepsis in a patient. In one embodiment, the minimum fold change increase or % increase for the pulmonary sepsis biomarkers is as defined above for the method for diagnosing pulmonary sepsis in a patient.

In one embodiment, the reference value used in the distinguishing method may include a reference value that is representative of an individual having pulmonary sepsis. All embodiments described above for the method of diagnosing abdominal sepsis when using a reference value that is representative of an individual having pulmonary sepsis apply equally to the distinguishing method described herein.

For example, an increase or decrease in the one or more biomarker for abdominal sepsis (and/or one or more additional biomarker) in the sample obtained from the patient relative to the corresponding reference value representative of an individual having pulmonary sepsis, indicates that the patient has or is at risk of developing abdominal sepsis. The increase or decrease may be as defined above for the method for diagnosing abdominal sepsis in a patient. No increase in the one or more biomarker for abdominal sepsis (and/or one or more additional biomarker) in the sample obtained from the patient relative to the corresponding reference value representative of an individual having pulmonary sepsis, indicates that the patient may have or is at risk of developing pulmonary sepsis (eg. where the patient has already been diagnosed as having sepsis).

In one embodiment, the reference value used in the distinguishing method may include a reference value that is representative of an individual having abdominal sepsis. All embodiments described above for the method of diagnosing pulmonary sepsis when using a reference value that is representative of an individual having abdominal sepsis apply equally to the distinguishing method described herein.

For example, an increase in the one or more biomarker for pulmonary sepsis (and/or one or more additional biomarker) in the sample obtained from the patient relative to the corresponding reference value representative of an individual having abdominal sepsis, indicates that the patient has or is at risk of developing pulmonary sepsis. The increase may be as defined above for the method for diagnosing pulmonary sepsis in a patient. No increase in the one or more biomarker for pulmonary sepsis (and/or one or more additional biomarker) in the sample obtained from the patient relative to the corresponding reference value representative of an individual having abdominal sepsis, indicates that the patient may have or is at risk of developing abdominal sepsis (eg. where the patient has already been diagnosed as having sepsis).

As described above for the “method for diagnosing a systemic inflammatory condition in a patient”, the method of the invention may involve the use of multiple separate reference values. All combinations of reference values defined above for the “method for diagnosing a systemic inflammatory condition in a patient” apply equally to the method for distinguishing between abdominal sepsis and pulmonary sepsis. For example, the reference value used in the method may comprise: (i) a reference value that is representative of an individual (or population of individuals) having abdominal sepsis and a separate reference value that is representative of an individual (or population of individuals) having pulmonary sepsis.

In one embodiment, the patient may be diagnosed as having abdominal sepsis and pulmonary sepsis when an increase is observed in any one or more of: MRAS, PCOLCE2, TMEM37, SLC39A8, KIF2C, CIQC, CIQB, CIQA, and TNF, and/or a decrease is observed in any one or more of: IF144, IFIT1, and RPGRIP1, in the sample obtained from the patient relative to the corresponding reference value representative of an individual having pulmonary sepsis; and an increase is observed in the one or more biomarker for pulmonary sepsis in the sample obtained from the patient relative to the corresponding reference value representative of an individual having abdominal sepsis.

The method for distinguishing between abdominal sepsis and pulmonary sepsis in a patient as described herein can be used in a decision tree process to investigate the health of a patient having or suspected of having a systemic inflammatory condition. For example, the method for distinguishing between abdominal sepsis and pulmonary sepsis in a patient can be performed before, after, or in addition to any of the other methods of the invention described herein.

In one embodiment, the method of the invention for distinguishing between abdominal sepsis and pulmonary sepsis in a patient can be performed subsequent to (or in addition to) the method for diagnosing whether a patient has a systemic inflammatory condition (as described herein). If the patient tests positive for a systemic inflammatory condition (using the method of the invention for diagnosing whether a patient has a systemic inflammatory condition), they may be tested using the distinguishing method described herein to determine whether they have abdominal sepsis and/or pulmonary sepsis.

In one embodiment, the method of the invention for distinguishing between abdominal sepsis and pulmonary sepsis in a patient can be performed subsequent to (or in addition to) the method for distinguishing between sepsis and SIRS in a patient (as described herein). If the patient tests positive for sepsis (using the method distinguishing between sepsis and SIRS in a patient), they may be tested using the distinguishing method described herein to determine whether they have abdominal sepsis and/or pulmonary sepsis.

In one embodiment, the method of the invention for distinguishing between abdominal sepsis and pulmonary sepsis in a patient can be performed subsequent to (or in addition to) the method for diagnosing sepsis in a patient (as described herein). If the patient tests positive for sepsis (using the method diagnosing sepsis), they may be tested using the distinguishing method described herein to determine whether they have abdominal sepsis and/or pulmonary sepsis.

In one embodiment, the method of the invention for distinguishing between abdominal sepsis and pulmonary sepsis in a patient can be performed subsequent to (or in addition to) the method for diagnosing whether a patient has a systemic inflammatory condition (as described herein), the method for distinguishing between sepsis and SIRS in a patient (as described herein), and/or the method for diagnosing sepsis (as described herein). If the patient tests positive for a systemic inflammatory condition (using the method of the invention for diagnosing whether a patient has a systemic inflammatory condition), they may be tested for sepsis using the method for distinguishing between sepsis and SIRS described herein, and/or the method for diagnosis of sepsis described herein. If the patients tests positive for sepsis, they may be tested using the distinguishing method described herein to determine whether they have abdominal sepsis and/or pulmonary sepsis.

In one embodiment, the patient may be tested for sepsis (using the method for distinguishing between sepsis and SIRS described herein, and/or the method for diagnosis of sepsis described herein). If the patient tests positive for sepsis, they may be tested using the distinguishing method described herein to determine whether they have abdominal sepsis and/or pulmonary sepsis.

The above described combination of methods may also be performed in parallel to determine the disease status of a patient by simultaneously (or substantially simultaneously) investigating the expression of all the biomarkers in a sample obtained from the patient, and determining whether the patient has or is at risk of having abdominal sepsis and/or pulmonary sepsis.

When performing these different methods in a decision tree process, the sample used in each step of the method may be the same sample obtained from the patient (as described herein). When the method comprises multiple quantification steps, these multiple steps may be performed at the same time (e.g. in parallel) and/or using the same sample. When the method comprises multiple comparison steps, these multiple steps may be performed at the same time (e.g. in parallel).

In a related aspect, the present invention also provides the use of one or more biomarker for abdominal sepsis (as described herein), and/or one or more biomarker for pulmonary sepsis (as described herein) for distinguishing between abdominal sepsis and pulmonary sepsis in a patient.

In one embodiment, the invention provides the use of one or more biomarker for abdominal sepsis selected from the group consisting of: SLC39A8, CIQC, CIQA, MRAS, TMEM37, CIQB, PCOLCE2, KIF2C TNF, IF144, IFIT1 and RPGRIP1, and/or one or more biomarker for pulmonary sepsis selected from the group consisting of: HCAR2, CXCR1, DISC1, EPSTI1, and IF144, for distinguishing between abdominal sepsis and pulmonary sepsis in a patient.

In one embodiment, the invention provides the use of one or more biomarker for abdominal sepsis selected from the group consisting of: SLC39A8, CIQC, CIQA, MRAS, TMEM37, CIQB, PCOLCE2, KIF2C and TNF, and/or one or more biomarker for pulmonary sepsis selected from the group consisting of: HCAR2, CXCR1, DISC1, EPSTI1, and IF144, for distinguishing between abdominal sepsis and pulmonary sepsis in a patient.

In one embodiment, the invention provides the use of one or more biomarker for abdominal sepsis selected from the group consisting of: SLC39A8, CIQC, CIQA, MRAS, TMEM37, and CIQB, and/or one or more biomarker for pulmonary sepsis selected from the group consisting of: HCAR2, CXCR1, and DISC1, for distinguishing between abdominal sepsis and pulmonary sepsis in a patient. For example, the abdominal sepsis biomarkers: SLC39A8, CIQC, CIQA, MRAS, TMEM37, and CIQB may be used in combination with the pulmonary sepsis biomarkers: HCAR2, CXCR1, and DISC1.

In one embodiment, the invention provides the use may be of one or more biomarker for abdominal sepsis selected from the group consisting of: SLC39A8, CIQC, and CIQA, and/or one or more biomarker for pulmonary sepsis selected from the group consisting of: HCAR2, CXCR1, and DISC1, for distinguishing between abdominal sepsis and pulmonary sepsis in a patient. For example, the abdominal sepsis biomarkers: SLC39A8, CIQC, and CIQA may be used in combination with the pulmonary sepsis biomarkers: HCAR2, CXCR1, and DISC1.

In one embodiment, the invention provides the use of one or more biomarker for abdominal sepsis selected from the group consisting of: SLC39A8, CIQC, and CIQA, and/or one or more biomarker for pulmonary sepsis selected from the group consisting of: HCAR2 and CXCR1, for distinguishing between abdominal sepsis and pulmonary sepsis in a patient. For example, the abdominal sepsis biomarkers: SLC39A8, CIQC, and CIQA may be used in combination with the pulmonary sepsis biomarkers: HCAR2, and CXCR1.

All embodiments described above for the “method of distinguishing between abdominal sepsis and pulmonary sepsis in a patient” apply equally to the ‘use’ of the invention described herein. This includes all embodiments relating to the terms “abdominal sepsis”, “pulmonary sepsis”, “patient”, “sample”, “the one or more biomarker for abdominal sepsis”, and “the one or more biomarker for pulmonary sepsis”. All combinations of abdominal sepsis and pulmonary sepsis biomarkers described above for the method of distinguishing between abdominal sepsis and pulmonary sepsis in a patient apply equally to the ‘use’ of the invention described herein.

### Monitoring a Systemic Inflammatory Condition

The progression of a patient from normalcy (ie. a condition characterised by not having a systemic inflammatory condition) to having a systemic inflammatory condition is characterised by changes in biomarkers, as certain biomarkers are expressed at increasingly higher levels and the expression of other biomarkers becomes down regulated. The present inventors have identified biomarkers that both increase and decrease in abundance as a physiological response to a systemic inflammatory condition is established or subsides. A feature of a patient's biomarker profile that is known to change in intensity as a physiological response to a systemic inflammatory condition becomes established may be therefore be selected for monitoring of a systemic inflammatory condition in a patient. A comparison of the same feature in a profile from a subsequent biological sample from the patient can establish whether the patient is developing a more severe form of the systemic inflammatory condition or is progressing towards normalcy. The present invention therefore also provides a method of monitoring a systemic inflammatory condition in a patient.

In one embodiment, the method of monitoring a systemic inflammatory condition comprises performing any of the methods of the invention for diagnosis of a systemic inflammatory condition (including those for diagnosis of sepsis, diagnosis of abdominal sepsis, diagnosis of pulmonary sepsis, diagnosis of SIRS, for distinguishing between sepsis and SIRS, and for distinguishing between abdominal sepsis and pulmonary sepsis, or any combination of these methods described herein) at a first time point, repeating the ‘quantification’ and ‘comparison’ steps of said method at one or more later time points, and comparing the presence and/or amount of each marker determined at said one or more later time point to the presence and/or amount of each marker determined at the first time point, to monitor the systemic inflammatory condition. All embodiments of the diagnostic methods described herein apply equally to the monitoring method of the invention.

By repeating the diagnostic method at one or more later time point, the disease status of the patient can be re-classified to determine whether there has been a change or no change in the disease status of the patient. For example, when the level of the one or more biomarker returns towards (or becomes increasingly statistically similar to) the level typically observed for the reference value representative of a healthy individual, and/or increasingly statistically deviates from the level typically observed for the reference value representative of a systemic inflammatory condition, this indicates that there has been an improvement or regression of the systemic inflammatory condition in the test individual. Likewise, when the level of the one or more biomarker increasingly statistically deviates from the level typically observed for the reference value representative of a healthy individual, and/or remains statistically similar to (or becomes increasingly statistically similar to) the level typically observed for the reference value representative of a systemic inflammatory condition, this indicates that there has been a worsening or progression of the systemic inflammatory condition in the test individual.

Monitoring of a systemic inflammatory condition in a patient may be used to monitor the recovery of a patient having a systemic inflammatory condition. As used herein, the term “recovery” refers to the survival of a patient having a systemic inflammatory condition. When a patient recovers from a systemic inflammatory condition, they no longer exhibit symptoms of the condition, and return to a normal (or near normal) state of health. In contrast, non-recovery from a systemic inflammatory condition means that the patient does not survive the systemic inflammatory condition. The symptoms of the condition in the patient generally worsen, and the patient may experience multiple organ failure resulting in death.

Monitoring of a systemic inflammatory condition in a patient may be used to monitor the severity of the systemic inflammatory condition in a patient. For example, the method of the invention may comprise monitoring of the progression, regression, aggravation, alleviation or recurrence of the condition. Monitoring of a systemic inflammatory condition in a patient may comprise determining whether the systemic inflammatory condition is progressing towards a more severe form of the condition, or regressing towards normalcy. Monitoring may also comprise determining whether the systemic inflammatory condition has remained stable.

As used herein, the term “progression” refers to an increase or worsening in the symptoms of a disease or disorder, and the term “regression” refers to a decrease or improvement in the symptoms of disease or disorder.

The monitoring method of the invention may be applied in the course of a medical treatment of the patient aimed at alleviating the monitored condition. In one embodiment, the monitoring method may be used to aid determination as to the correct course of treatment, permit evaluation of the effectiveness of treatment, and/or permit determination as to whether to continue or cease treatment. In a preferred embodiment, the method is used to monitor the effectiveness of a treatment regimen for a systemic inflammatory condition. Suitable therapies are as described herein for the treatment of sepsis and/or SIRS.

The monitoring method of the invention may also be used to make decisions about a patient, such as deciding whether a patient may be discharged, needs a change in treatment or needs further hospitalisation.

The monitoring method of the invention may be used to provide a means of disease staging and/or to permit determination as to clinical outcome. In one embodiment, the method may be used to monitor a patient for prognosis of recovery.

As used herein, the terms “prognosis” or “prognosticating” refers to an anticipation on the progression of a disease or condition and the prospect of recovery. A “good prognosis” (or a “prognosis of recovery”) refers to an anticipation of a satisfactory partial or complete recovery from the disease or condition. A “poor prognosis” (or a “prognosis of non-recovery”) encompasses anticipation of a substandard recovery and/or unsatisfactory recovery, or to substantially no recovery, or even further worsening of the disease or condition.

Monitoring of a systemic inflammatory condition can also be performed without an external reference value, by obtaining samples from the patient at different time points, and comparing the marker profile of these samples to one another.

In one embodiment, the method for monitoring a systemic inflammatory condition in a patient, comprises:
(i) determining the presence and/or amount of one or more biomarker described herein in a sample obtained from a patient at a first (or earlier) time point;
(ii) determining the presence and/or amount of the one or more biomarker in a sample obtained from the patient at one or more later time points;
(iii) comparing the presence and/or amount of the one or more biomarker determined in step (ii) to the presence and/or amount of the one or more biomarker determined in step (i).

The “systemic inflammatory condition” monitored using the method of the invention is as described above for the diagnostic methods described herein. In one embodiment, the systemic inflammatory condition is selected from one or more (eg. both) of SIRS and sepsis. In one embodiment, the systemic inflammatory condition is selected from one or more (eg. two or more or all 3) of SIRS, abdominal sepsis and pulmonary sepsis. In one embodiment, the systemic inflammatory condition is SIRS. In one embodiment, the systemic inflammatory condition is sepsis. In one embodiment, the systemic inflammatory condition is abdominal sepsis. In one embodiment, the systemic inflammatory condition is pulmonary sepsis.

In one embodiment, steps (i) and (ii) of the method involve “determining the presence and amount of the one or more biomarker in a sample obtained from a patient”, and step (iii) involves “comparing the presence and amount of the one or more biomarker determined in step (ii) to the presence and amount of the one or more biomarker determined in step (i)”. In one embodiment, steps (i) and (ii) of the method involve “determining the amount of the one or more biomarker in a sample obtained from a patient”, and step (iii) involves “comparing the amount of the one or more biomarker determined in step (ii) to the amount of the one or more biomarker determined in step (i)”.

The “patient” for which monitoring is performed is as defined above for the diagnostic methods described herein. In one embodiment, the patient is suspected of having or being at risk of developing a systemic inflammatory condition. In one embodiment, the patient has been diagnosed as having or being at risk of developing a systemic inflammatory condition. In one embodiment, the patient has been diagnosed as having or being at risk of developing a systemic inflammatory condition using the method described herein for diagnosing a systemic inflammatory condition. In one embodiment, the patient has been diagnosed as having or being at risk of developing a systemic inflammatory condition using the methods described herein for diagnosing SIRS, sepsis (such as abdominal sepsis, or pulmonary sepsis), or using the method described herein for distinguishing between sepsis and SIRS in a patient, or for distinguishing between abdominal sepsis and pulmonary sepsis, or any combination of these methods described herein.

In one embodiment, the patient has been diagnosed as having SIRS (eg. using the method described herein for diagnosis of SIRS, or for distinguishing between sepsis and SIRS in a patient). In one embodiment, the patient has been diagnosed as having sepsis, such as abdominal sepsis or pulmonary sepsis (eg. using the methods described herein for diagnosis of sepsis, abdominal sepsis or pulmonary sepsis, the method described herein for distinguishing between sepsis and SIRS in a patient, or for distinguishing between abdominal sepsis and pulmonary sepsis, or any combination of these methods described herein). The patient may be undergoing treatment for a systemic inflammatory condition.

The “sample” obtained from the patient is as defined above for the diagnostic methods.

The monitoring methods described herein allow the monitoring of a systemic inflammatory condition in a patient over time. All embodiments relating to the time point at which a sample is obtained from the patient as described above for the diagnostic methods (eg. in the method for diagnosing a systemic inflammatory condition in a patient) apply equally to the sample obtained from the patient at “a first (or earlier) time point” in the monitoring methods described herein. For example, the sample may be obtained at least 1 hour, 2 hours, 4 hours, 6 hours, 8 hours, 12 hours, 36 hours, 48 hours, 72 hours, 96 hours, or 120 hours, after the patient presents with one or more clinical symptoms of a systemic inflammatory condition, or is admitted to a medical care facility. In one embodiment, the sample may be obtained from the patient at least 24 hours after the patient presents with one or more clinical symptoms of a systemic inflammatory condition, or is admitted to a medical care facility.

The sample obtained from the patient at the “one or more later time points” may be obtained at least 6 hours (e.g. at least 12 hours, at least 18 hours, at least 24 hours, at least 48 hours, at least 72 hours, at least 96 hours, at least 120 hours, at least 1 week, at least 2 weeks, at least 3 weeks, at least 1 month) after the sample was obtained from the patient at a first (or earlier) time point.

In one embodiment, when the method is for monitoring the effectiveness of a treatment regimen for a systemic inflammatory condition in a patient, the sample obtained from the patient at a first (or earlier) time point is obtained from the patient before or during the course of treatment. For example, the sample may be obtained from the patient at least 1 hour (e.g. at least 2 hours, at least 4 hours, at least 8 hours, at least 12 hours, at least 18 hours, at least 24 hours) before treatment. The sample obtained from the patient at one or more later time points is obtained during or after a course of treatment. For example, the sample may be obtained from the patient at least 1 hour (e.g. at least 2 hours, at least 4 hours, at least 8 hours, at least 12 hours, at least 18 hours, at least 24 hours) after a treatment regimen has begun or has been completed.

The “one or more biomarker” of the invention is as described above for the diagnostic methods described herein. The one or more biomarker used in the monitoring method of the invention may include any of the biomarkers described herein (e.g. as defined in Tables 1-4), or any combination of biomarkers described herein.

In addition to the biomarkers described above, the present inventors have also identified a set of biomarkers which are particularly useful for monitoring a systemic inflammatory condition in a patient. These biomarkers include ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1 and LILRB5 (see Tables 1 and 4).

Thus, in one embodiment, the one or more biomarker is selected from: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1, and LILRB5. These biomarkers were observed to change in abundance over time in the samples obtained from patients having systemic inflammatory conditions (such as sepsis or SIRS), as the patients either recovered from the condition, or did not recover and subsequently died.

The reference to the biomarker HLA-DPB1 throughout the entire description, includes the HLA-DPB1 sequence encoded by SEQ ID NO: 30 and the transcript variant X1 of HLA-DPB1 (as encoded by SEQ ID NO:31). In one embodiment, the reference to the biomarker HLA-DPB1 is a reference to sequence encoded by SEQ ID NO: 30. In one embodiment, the reference to the biomarker HLA-DPB1 is a reference to the transcript variant X1 of HLA-DPB1 (as encoded by SEQ ID NO: 31).

Each of the biomarkers may be used alone, or in combination with any of the biomarkers described herein to monitor a systemic inflammatory condition in the method of the invention. For example, any combination of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, up to and including all of the biomarkers may be used to monitor a systemic inflammatory condition in a patient.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, or all 21) of the biomarkers selected from the group consisting of: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1, and LILRB5, may be used to monitor a systemic inflammatory condition in a patient (such as abdominal sepsis and/or SIRS). For example, any combination of 1 or more (eg. 2 or more, 3 or more, 4 or more, or all 5) of the biomarkers selected from the group consisting of: KLRB1, BCL11B, FCER1A, PKHD1, and LILRB5, may be used to monitor a systemic inflammatory condition in a patient (such as sepsis and/or SIRS).

A sub-set of these biomarkers is particularly useful for monitoring sepsis (eg. abdominal sepsis), including ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, and MX1. Thus, in one embodiment, the one or more biomarker is selected from: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, and MX1. In one embodiment, the method of the invention is for monitoring of sepsis (eg. abdominal sepsis) in a patient.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or all 18) of the biomarkers selected from the group consisting of: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, and MX1, may be used to monitor a systemic inflammatory condition in a patient (such as abdominal sepsis).

Within this sub-set of biomarkers, the inventors identified markers that increase in abundance over time as the patient recovers from abdominal sepsis (returning towards the elevated level typically observed for healthy individuals), but show no increase (or a decrease) in abundance when the patient does not recover from abdominal sepsis. These include one or more biomarker selected from: ITM2A, CCL5, KLRK1, KLRB1, HLA-DRA, BCL11B, HLA-DPB1, SLAMF6, CD160, KLRF1, and MX1. The inventors also identified that the biomarker NPPC increases significantly in abundance over time when the patient does not recover from abdominal sepsis, and show no increase (or a decrease) in abundance over time when the patient does recover from abdominal sepsis (thereby returning towards the reduced level typically observed for healthy individuals). These biomarkers are particularly useful for monitoring abdominal sepsis in a patient, particularly for monitoring recovery from abdominal sepsis.

In one embodiment, the one or more biomarker is selected from: ITM2A, CCL5, NPPC, KLRK1, KLRB1, HLA-DRA, BCL11B, HLA-DPB1, SLAMF6, CD160, KLRF1, and MX1. In one embodiment, any combination of 1 or more (eg. 2 or more, 3 or more, 4 or more, 5 or more, 6 or more, 7 or more, 8 or more, 9 or more, 10 or more, 11 or more, or all 12) of the biomarkers selected from the group consisting of: ITM2A, CCL5, NPPC, KLRK1, KLRB1, HLA-DRA, BCL11B, HLA-DPB1, SLAMF6, CD160, KLRF1, and MX1, may be used to monitor a systemic inflammatory condition in a patient (such as abdominal sepsis).

In one embodiment, the one or more biomarker is selected from one or more (eg. 2 or more, 3 or more, 4 or more, 5 or more, 6 or more, 7 or more, 8 or more, 9 or more, 10 or more, or all 11) of: ITM2A, CCL5, KLRK1, KLRB1, HLA-DRA, BCL11B, HLA-DPB1, SLAMF6, CD160, KLRF1, and MX1, in combination with the biomarker NPPC.

The inventors also observed that a sub-set of these biomarkers is particularly useful for monitoring SIRS, including ITM2A, CCL5, NPPC, PKD1, KLRK1, KLRB1, HLA-DRA, BCL11B, HLA-DPB1, CD160, KLRF1, CD2, LGALS2, MYCL, NECAB1, and PKHD1. Thus, in one embodiment, the one or more biomarker is selected from: ITM2A, CCL5, NPPC, PKD1, KLRK1, KLRB1, HLA-DRA, BCL11B, HLA-DPB1, CD160, KLRF1, CD2, LGALS2, MYCL, NECAB1, and PKHD1. In one embodiment, the method of the invention is for monitoring of SIRS in a patient.

In one embodiment, any combination of 1 or more (eg. 2 or more, 3 or more, 4 or more, 5 or more, 6 or more, 7 or more, 8 or more, 9 or more, 10 or more, 11 or more, 12 or more, 13 or more, 14 or more, 15 or more, or all 16) of the biomarkers selected from the group consisting of: ITM2A, CCL5, NPPC, PKD1, KLRK1, KLRB1, HLA-DRA, BCL11B, HLA-DPB1, CD160, KLRF1, CD2, LGALS2, MYCL, NECAB1, and PKHD1, may be used to monitor a systemic inflammatory condition in a patient (such as SIRS).

Within this sub-set of biomarkers, the inventors further identified a sub-set of markers that increase in abundance over time as the patient recovers from SIRS (returning towards the elevated levels typically observed for healthy individuals), but show no increase (or a decrease) in abundance when the patient does not recover from SIRS. These include one or more biomarker selected from: ITM2A, CCL5, KLRK1, KLRB1, and BCL11B. The inventors also identified that the biomarkers NPPC, PKDI, CD2, LGALS2, MYCL, NECAB1, and PKHD1 increase significantly in abundance over time when the patient does not recover from SIRS, and show no increase (or a decrease) in abundance over time when the patient does recover from SIRS (thereby returning towards the reduced level typically observed for healthy individuals). These biomarkers are particularly useful for monitoring SIRS in a patient, particularly for monitoring recovery from SIRS.

In one embodiment, the one or more biomarker is selected from: ITM2A, CCL5, NPPC, PKD1, KLRK1, KLRB1, BCL11B, CD2, LGALS2, MYCL, NECAB1, and PKHD1. In one embodiment, any combination of 1 or more (eg. 2 or more, 3 or more, 4 or more, 5 or more, 6 or more, 7 or more, 8 or more, 9 or more, 10 or more, 11 or more or all 12) of the biomarkers selected from the group consisting of: ITM2A, CCL5, NPPC, PKD1, KLRK1, KLRB1, BCL11B, CD2, LGALS2, MYCL, NECAB1, and PKHD1, may be used to monitor a systemic inflammatory condition in a patient (such as SIRS).

For example, the one or more biomarker may be selected from: ITM2A, CCL5, NPPC, PKD1, LGALS2, MYCL, NECAB1, and PKHD1. For example, the one or more biomarker may be selected from: CCL5, NPPC, PKD1, LGALS2, MYCL, NECAB1, and PKHD1. For example, the one or more biomarker may be selected from: CCL5, NPPC, PKD1, LGALS2, NECAB1, and PKHD1. For example, the one or more biomarker may be selected from: CCL5, NPPC, PKDI, NECAB1, and PKHD1.

In one embodiment, the one or more biomarker is selected from one or more of: ITM2A, CCL5, KLRK1, KLRB1, and BCL11B, in combination with one or more biomarker selected from: NPPC, PKDI, CD2, LGALS2, MYCL, NECAB1, and PKHD1. For example, the one or more biomarker may be selected from one (eg. both) or more of: ITM2A and CCL5, in combination with one or more (eg. 2 or more, 3 or more, or all 4) biomarker selected from: NPPC, PKDI, NECAB1, and PKHD1. For example, the one or more biomarker may be CCL5 used in combination with one or more biomarker selected from: NPPC, PKD1, LGALS2, MYCL, NECAB1, and PKHD1. For example, the one or more biomarker may be CCL5 used in combination with one or more biomarker selected from: NPPC, PKD1, NECAB1, and PKHD1.

The present inventors have identified biomarkers that can be used to monitor multiple different types of systemic inflammatory condition (such SIRS and sepsis) in a single method. In one embodiment, the one or more (eg. 2 or more, 3 or more, 4 or more, 5 or more, 6 or more, 7 or more, or all 8) biomarker is selected from: ITM2A, CCL5, NPPC, KLRK1, KLRB1, HLA-DRA, BCL11B, and HLA-DPB1. In one embodiment, the one or more (2 or more, 3 or more, 4 or more, or all 5) biomarker is selected from: ITM2A, CCL5, NPPC, KLRK1, and BCL11B. In one embodiment, the one or more (2 or more, or all 3) biomarker is selected from: ITM2A, CCL5, and NPPC. For example, the one or more biomarker is selected from: CCL5 and NPPC. In one embodiment, the one or more biomarker may be one or more (or both) of: ITM2A and CCL5 in combination with NPPC.

In one embodiment, the one or more biomarker is FCER1A. In one embodiment, the one or more biomarker is KLRK1. In one embodiment, the one or more biomarker is KLRB1. In one embodiment, the one or more biomarker is DAAM2. In one embodiment, the one or more biomarker is HLA-DRA. In one embodiment, the one or more biomarker is BCL11B. In one embodiment, the one or more biomarker is SLAMF6. In one embodiment, the one or more biomarker is ITM2A. In one embodiment, the one or more biomarker is CD160. In one embodiment, the one or more biomarker is HLA-DPB1. In one embodiment, the one or more biomarker is KLRF1. In one embodiment, the one or more biomarker is CD2. In one embodiment, the one or more biomarker is LGALS2. In one embodiment, the one or more biomarker is NPPC. In one embodiment, the one or more biomarker is MYCL. In one embodiment, the one or more biomarker is MX1. In one embodiment, the one or more biomarker is NECAB1. In one embodiment, the one or more biomarker is NECAB2. In one embodiment, the one or more biomarker is PKHD1. In one embodiment, the one or more biomarker is PKD1. In one embodiment, the one or more biomarker is CCL5. In one embodiment, the one or more biomarker is LILRB5.

All embodiments of the ‘quantification’ and ‘comparison’ steps of the diagnostic methods described herein (such as the method for diagnosis of a systemic inflammatory condition) apply equally to the monitoring methods described herein.

The step of “comparing the presence and/or amount determined in step (ii) to the presence and/or amount determined in step (i)” involves determining whether there is a difference in the presence and/or amount of the one or more biomarkers between the samples. It is possible to monitor a systemic inflammatory condition by attributing the finding of a difference or no difference in the one or more biomarker to a change in the systemic inflammatory condition in the individual between the two or more successive time points.

A finding of “no difference” in the presence and/or amount of the one or more biomarker detected in the two or more successive time points indicates that there has been no change in the systemic inflammatory condition in the individual. In contrast, finding of a “difference” in the presence and/or amount of the one or more biomarker detected in the two or more successive time points indicates that there has been a change in the systemic inflammatory condition in the individual.

A difference in the presence and/or amount of the one or more biomarker measured by the monitoring methods of the present invention can comprise an increase or decrease in the one or more biomarkers over time. The increase or decrease in the biomarker can be, for example, at least 0.1 (eg. at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, or at least 10) fold over time. The difference in the presence and/or amount of the biomarker is preferably statistically significant. By “statistically significant”, it is meant that the alteration is greater than what might be expected to happen by chance alone.

The increase or decrease in the one or more biomarker in the patient over time can indicate progression of the disease, the lack of efficacy of one or more treatment regimens, and/or a poor prognosis of recovery (or a prognosis of non-recovery). Alternatively, the increase or decrease in the one or more biomarker in the patient over time can indicate regression of the disease, the success of one or more treatment regimens, and/or a good prognosis of recovery (or a prognosis of recovery).

For example, an increase in any one or more of ITM2A, CCL5, KLRK1, KLRB1, HLA-DRA, BCL11B, HLA-DPB1, SLAMF6, CD160, KLRF1, and MX1 in the sample obtained at the later time point relative to the sample obtained from the first time point indicates regression of the systemic inflammatory condition in the patient. No increase in ITM2A, CCL5, KLRK1, KLRB1, HLA-DRA, BCL11B, HLA-DPB1, SLAMF6, CD160, KLRF1, or MX1 in the sample obtained at the later time point relative to the sample obtained from the first time indicates no regression of the systemic inflammatory condition in the patient. In one embodiment, no increase in ITM2A, CCL5, KLRK1, KLRB1, HLA-DRA, BCL11B, HLA-DPB1, SLAMF6, CD160, KLRF1, or MX1 in the sample obtained at the later time point relative to the sample obtained from the first time point may indicate progression of the systemic inflammatory condition in the patient.

For example, an increase in any one or more of ITM2A, CCL5, KLRK1, KLRB1, HLA-DRA, BCL11B, HLA-DPB1, SLAMF6, CD160, KLRF1, and MX1 in the sample obtained at the later time point relative to the sample obtained from the first time point indicates the success of one or more treatment regimens. No increase in ITM2A, CCL5, KLRK1, KLRB1, HLA-DRA, BCL11B, HLA-DPB1, SLAMF6, CD160, KLRF1, or MX1 in the sample obtained at the later time point relative to the sample obtained from the first time point indicates the lack of efficacy of one or more treatment regimens.

For example, an increase in any one or more of ITM2A, CCL5, KLRK1, KLRB1, HLA-DRA, BCL11B, HLA-DPB1, SLAMF6, CD160, KLRF1, and MX1 in the sample obtained at the later time v relative to the sample obtained from the first time point indicates a (good) prognosis of recovery. No increase in ITM2A, CCL5, KLRK1, KLRB1, HLA-DRA, BCL11B, HLA-DPB1, SLAMF6, CD160, KLRF1, or MX1 in the sample obtained at the later time point relative to the sample obtained from the first time point indicates a poor prognosis of recovery (or a prognosis of non-recovery).

In a further example, an increase in any one or more of NPPC, PKD1, CD2, LGALS2, MYCL, NECAB1, and PKHD1 in the sample obtained at the later time point relative to the sample obtained from the first time point indicates progression of the systemic inflammatory condition in the patient. No increase in NPPC, PKD1, CD2, LGALS2, MYCL, NECAB1, or PKHD1, in the sample obtained at the later time point relative to the sample obtained from the first time point indicates no progression of the systemic inflammatory condition in the patient. In one embodiment, no increase in NPPC, PKD1, CD2, LGALS2, MYCL, NECAB1, or PKHD1, in the sample obtained at the later time point relative to the sample obtained from the first time point indicates regression of the systemic inflammatory condition in the patient.

In a further example, an increase in any one or more of NPPC, PKD1, CD2, LGALS2, MYCL, NECAB1, and PKHD1 in the sample obtained at the later time point relative to the sample obtained from the first time point indicates the lack of efficacy of one or more treatment regimens. No increase in NPPC, PKD1, CD2, LGALS2, MYCL, NECAB1, or PKHD1 in the sample obtained at the later time point relative to the sample obtained from the first time point indicates the success of one or more treatment regimens.

In a further example, an increase in any one or more of NPPC, PKD1, CD2, LGALS2, MYCL, NECAB1, and PKHD1 in the sample obtained at the later time point relative to the sample obtained from the first time point indicates a poor prognosis of recovery (or a prognosis of non-recovery). No increase in NPPC, PKD1, CD2, LGALS2, MYCL, NECAB1, and PKHD1 in the sample obtained at the later time point relative to the sample obtained from the first time point indicates a (good) prognosis of recovery.

In a related aspect, the present invention also provides the use of the one or more biomarker described herein for monitoring a systemic inflammatory condition in a patient.

In one embodiment, the use is of one or more biomarker selected from: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, PKHD1, and LILRB5 for monitoring a systemic inflammatory condition in a patient.

In one embodiment, the use is of one or more biomarker selected from ITM2A, CCL5, NPPC, KLRK1, KLRB1, HLA-DRA, BCL11B, and HLA-DPB1, for monitoring a systemic inflammatory condition in a patient (such sepsis and/or SIRS). For example, the one or more biomarker may be selected from: ITM2A, CCL5, NPPC, KLRK1, and BCL11B. For example, the one or more biomarker may be selected from: ITM2A, CCL5, and NPPC.

In one embodiment, the use is of one or more biomarker selected from: ITM2A, CCL5, NPPC, KLRK1, KLRB1, HLA-DRA, BCL11B, HLA-DPB1, FCER1A, DAAM2, SLAMF6, CD160, KLRF1, CD2, LGALS2, MYCL, and MX1, for monitoring a systemic inflammatory condition in a patient (such as abdominal sepsis).

For example, the one or more biomarker may be selected from: ITM2A, CCL5, NPPC, KLRK1, KLRB1, HLA-DRA, BCL11B, HLA-DPB1, SLAMF6, CD160, KLRF1, and MX1.

In one embodiment, the use is of one or more biomarker selected from: ITM2A, CCL5, NPPC, PKD1, KLRK1, KLRB1, HLA-DRA, BCL11B, HLA-DPB1, CD160, KLRF1, CD2, LGALS2, MYCL, NECAB1, and PKHD1 for monitoring a systemic inflammatory condition in a patient (such as SIRS). In one embodiment, the use is of one or more biomarker selected from: ITM2A, CCL5, NPPC, PKD1, KLRK1, KLRB1, BCL11B, CD2, LGALS2, MYCL, NECAB1, and PKHD1 for monitoring a systemic inflammatory condition in a patient (such as SIRS). For example, the one or more biomarker may be selected from: CCL5, NPPC, PKD1, LGALS2, MYCL, NECAB1, and PKHD1.

All embodiments described above for the method of monitoring a systemic inflammatory condition in a patient apply equally to the ‘use’ of the invention described herein. This includes all embodiments relating to the terms “systemic inflammatory condition”, “patient”, “sample obtained a first time point”, “sample obtained at one or more later time points”, and “the one or more biomarker”.

### Survival Biomarkers

A major issue facing clinicians is determining when a patient is suitable for release from medical care. In some cases, patients appear to physically recover (eg. from a systemic inflammatory condition), yet still do not survive after they are discharged from medical care. When studying the gene expression patterns of biomarkers in patients having a systemic inflammatory condition, the inventors surprisingly observed that several of the biomarkers described herein were present at much higher levels in patients that did not survive as compared to patients that made a full recovery. The inventors observed that the likelihood of survival of a patient could therefore be predicted by monitoring the levels of these “survival” biomarkers. Detection of the levels of these biomarkers in patients will therefore assist clinicians in determining whether a patient is suitable for discharge from medical care.

The present invention therefore provides a method for determining whether a patient is suitable for discharge from medical care, comprising:

(i) determining the presence and/or amount of one or more biomarker selected from the group consisting of: NECAB1, NECAB2, PKD1, PKHD1, LILRB4, and LILRB5 in a sample obtained from a patient,
(ii) comparing the presence and/or amount of the one or more biomarker determined in said sample in (i) to a corresponding reference value, and thereby determining whether the patient is suitable for discharge from medical care.

“Determining whether a patient is suitable for discharge from medical care” means determining whether the patient has a good prognosis of survival and can be safely discharged from medical care. The method therefore provides a way of predicting the survival of a patient (such as a patient that has been diagnosed with a systemic inflammatory condition). The method may therefore be alternatively defined as a “method for predicting the survival of a patient”.

As used herein, “discharge from medical care” encompasses “discharge from high-dependency medical care”. For example, it may refer to the act of moving a patient from a high dependency unit (such as an intensive care unit) to a lower dependency unit (such as an outpatient unit, a hospital ward, or home care).

In one embodiment, step (i) of the method involves “determining the presence and amount of the one or more biomarker in a sample obtained from a patient”, and step (ii) involves “comparing the presence and amount of the one or more biomarker determined in said sample in (i) to a corresponding reference value”. In one embodiment, step (i) of the method involves “determining the amount of the one or more biomarker in a sample”, and step (ii) involves “comparing the amount of the one or more biomarker determined in said sample in (i) to a corresponding reference value”.

The “sample” obtained from the patient is as defined above for the diagnostic methods and monitoring methods described herein, including all embodiments relating to the time point at which the sample is obtained. In one embodiment, the sample may be obtained at least 48 hours after the patient presents with one or more clinical symptoms of a systemic inflammatory condition, or is admitted to a medical care facility. In one embodiment, the sample may be obtained at least 72 hours after the patient presents with one or more clinical symptoms of a systemic inflammatory condition, or is admitted to a medical care facility. In one embodiment, the sample may be obtained at least 96 hours after the patient presents with one or more clinical symptoms of a systemic inflammatory condition, or is admitted to a medical care facility. In one embodiment, the sample may be obtained at least 120 hours after the patient presents with one or more clinical symptoms of a systemic inflammatory condition, or is admitted to a medical care facility.

The method of the invention is intended to be used as a point of care monitor to determine whether it is safe to discharge a patient from medical care. In one embodiment, the sample may be obtained from a patient after treatment for a systemic inflammatory condition has been completed. In one embodiment, the sample is obtained from a patient when they have been clinically diagnosed as being suitable for discharge from medical care.

The “patient” is as described above for the diagnostic methods and monitoring methods described herein. In one embodiment, the patient has been diagnosed as having or being at risk of developing a systemic inflammatory condition. In one embodiment, the patient has been diagnosed as having or being at risk of developing a systemic inflammatory condition using the method described herein for diagnosing a systemic inflammatory condition. In one embodiment, the patient may have been diagnosed as having or being at risk of developing a systemic inflammatory condition using the methods described herein for diagnosing SIRS, sepsis (such as abdominal sepsis or pulmonary sepsis), or using the method described herein for distinguishing between sepsis and SIRS in a patient, or any combination of these methods as described herein). The patient may be undergoing (or has undergone) treatment for a systemic inflammatory condition.

In one embodiment, the patient has been diagnosed as having or being at risk of developing SIRS (eg. using any of the methods described herein for diagnosing SIRS, or for distinguishing between sepsis and SIRS in a patient). The patient may be undergoing (or has undergone) treatment for SIRS.

In one embodiment, the patient has been diagnosed as having or being at risk of developing sepsis (eg. using any of the methods described herein for diagnosing sepsis, or for distinguishing between sepsis and SIRS in a patient). The patient may be undergoing (or has undergone) treatment for sepsis.

In one embodiment, the patient has been diagnosed as having or being at risk of developing abdominal sepsis (eg. using the method described herein for diagnosing abdominal sepsis). The patient may be undergoing (or has undergone) treatment for abdominal sepsis.

In one embodiment, the patient has been diagnosed as having or being at risk of developing pulmonary sepsis (eg. using the method described herein for diagnosing pulmonary sepsis). The patient may be undergoing (or has undergone) treatment for pulmonary sepsis.

The “one or more biomarker” of the invention is as described above for the diagnostic methods and monitoring methods described herein. In one embodiment, the one or more biomarker may be selected from the group consisting of: NECAB1, NECAB2, PKD1, PKHD1, LILRB4, and LILRB5.

Each of the biomarkers may be used alone, or in combination with any of the survival biomarkers described herein in the method of the invention. For example, any combination of 1 or more, 2 or more, 3 or more, 4 or more, 5 or more or all 6 of the biomarkers may be used in the method of the invention.

In one embodiment, any combination of 1 or more (eg. 2 or more, 3 or more, 4 or more, 5 or more, or all 6) of the biomarkers selected from the group consisting of: NECAB1, NECAB2, PKD1, PKHD1, LILRB4, and LILRB5, may be used to determine whether a patient is suitable for discharge from medical care.

A combination of the ‘survival’ biomarkers may be used in the method to determine whether a patient is suitable for discharge from medical care. In one embodiment, the method may involve determining the presence and/or amount of one or more biomarker selected from NECAB2 and PKD1, in combination with one or more biomarker selected from: NECAB1, PKDI, PKHD1, LILRB4 and LILRB5. For example, the combination of biomarkers used in the method may be NECAB2 and NECAB1. For example, the combination of biomarkers used in the method may be NECAB2 and PKHD1. For example, the combination of biomarkers used in the method may be NECAB2 and PKD1. For example, the combination of biomarkers used in the method may be NECAB2 and LILRB4. For example, the combination of biomarkers used in the method may be NECAB2 and LILRB5. For example, the combination of biomarkers used in the method may be PKD1 and PKHD1. For example, the combination of biomarkers used in the method may be PKD1 and NECAB1. For example, the combination of biomarkers used in the method may be PKD1 and LILRB4. For example, the combination of biomarkers used in the method may be PKD1 and LILRB5.

In one embodiment, the one or more biomarker is NECAB1. In one embodiment, the one or more biomarker is NECAB2. In one embodiment, the one or more biomarker is PKD1. In one embodiment, the one or more biomarker is PKHD1. In one embodiment, the one or more biomarker is LILRB4. In one embodiment, the one or more biomarker is LILRB5.

The biomarkers NECAB1 and NECAB2 are brain specific markers, and the biomarkers PKHD1 and PKD1 are kidney specific markers. These markers are not usually expressed in peripheral blood leukocytes. The high levels of these markers in the patients that did not survive indicates that these patients are suffering from kidney damage and/or brain damage. The method described herein may therefore be used to diagnose organ damage in a patient. In one embodiment, the method is for diagnosis of organ damage in a patient. For example, the method may be for diagnosis of brain damage in a patient, when the one or more biomarker is selected from NECAB1 and/or NECAB2. By performing steps (i) and (ii) of the method described herein, the method can be used to determine whether a patient has or is at risk of developing brain damage. The method may alternatively be for diagnosis of kidney damage in a patient, when the one or more biomarker is selected from PKHD1 and/or PKD1. By performing steps (i) and (ii) of the method described herein, the method can be used to determine whether a patient has or is at risk of developing kidney damage.

The present inventors have observed that a sub-set of these biomarkers (NECAB2, PKD1, PKHD1 and LILRB5,) are particularly useful in determining whether a patient diagnosed as having or being at risk of developing sepsis (such as abdominal sepsis and/or pulmonary sepsis) is suitable for discharge from medical care. In one embodiment, the one or more biomarkers may be selected from the group consisting of NECAB2, LILRB5, PKHD1 and PKD1. The patient may be undergoing (or has undergone) treatment for sepsis (such as abdominal sepsis and/or pulmonary sepsis). Treatment for sepsis is as described herein.

A subset of the biomarkers (NECAB2 and PKD1) are particularly useful in determining whether a patient diagnosed as having or being at risk of developing abdominal sepsis is suitable for discharge from medical care. As described in Example 2, ROC analysis demonstrated that these biomarkers could be used alone or in combination to effectively distinguish between abdominal sepsis patients that died and those that survived. In one embodiment, the one or more biomarkers may be selected from the group consisting of NECAB2 and PKD1. For example, the method may be performed using the combination of biomarkers NECAB2 and PKD1. For example, the method may be performed using NECAB2. For example, the method may be performed using PKD1. The patient may be undergoing (or has undergone) treatment for abdominal sepsis.

A subset of the biomarkers (PKHD1 and LILRB5) are particularly useful in determining whether a patient diagnosed as having or being at risk of developing pulmonary sepsis is suitable for discharge from medical care. As described in Example 2, ROC analysis demonstrated that these biomarkers could be used alone or in combination to effectively distinguish between pulmonary sepsis patients that died and those that survived. In one embodiment, the one or more biomarkers may be selected from the group consisting of PKHD1 and LILRB5. For example, the method may be performed using the combination of biomarkers PKHD1 and LILRB5. For example, the method may be performed using PKHD1. For example, the method may be performed using LILRB5. The patient may be undergoing (or has undergone) treatment for pulmonary sepsis.

The present inventors have observed that a sub-set of these biomarkers (NECAB1, PKDI, PKHD1, LILRB4, and LILRB5) are particularly useful in determining whether a patient diagnosed as having or being at risk of developing SIRS is suitable for discharge from medical care. The patient may be undergoing (or has undergone) treatment for SIRS. Treatment for SIRS is as described herein. Thus, in one embodiment, the one or more biomarker is selected from the group consisting of: NECAB1, PKDI, PKHD1, LILRB4, and LILRB5. In one embodiment, any combination of 1 or more (eg. 2 or more, 3 or more, 4 or more, or all 5) of the biomarkers selected from the group consisting of: NECAB1, PKD1, PKHD1, LILRB4, and LILRB5, may be used to determine whether a patient is suitable for discharge from medical care.

As described in Example 2, PKHD1 and NECAB1 were observed to provide the most accurate distinction between patients with SIRS that survived and those that died (see the ROC curve data in Example 2). Good results were observed when these markers were used on their own or in combination. In one embodiment, the one or more biomarker may be PKHD1 and/or NECAB1. For example, the markers for determining whether a patient diagnosed as having or being at risk of developing SIRS is suitable for discharge from medical care may comprise the combination of PKHD1 and NECAB1.

All embodiments of the ‘quantification’ and ‘comparison’ steps described above for the diagnostic and monitoring methods described herein apply equally to the method for determining whether a patient is suitable for discharge from medical care. This includes all embodiments relating to the “reference value”.

The one or more biomarker measured by the methods of the present invention may increase or decrease as compared to the corresponding reference value. The increase or decrease in the amount of the one or more biomarker in the patient as compared to the reference value can indicate that the patient has a good prognosis of recovery (or survival) from the systemic inflammatory condition, and thus is suitable for discharge from medical care. Alternatively, the increase or decrease in the one or more biomarker in the patient as compared to the reference can indicate that the patient has a poor prognosis of recovery (or survival) (or a prognosis of non-recovery) from the systemic inflammatory condition, and thus is not suitable for discharge from medical care.

The increase or decrease in the one or more biomarker as compared to the reference can be, for example, at least 0.1 (eg. at least 0.2, at least 0.3, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.5, at least 3, at least 3.5, at least 4, at least 4.5, at least 5, at least 5.5, at least 6, at least 7 fold, at least 7.5, at least 8, at least 8.5, at least 9, at least 9.5, or at least 10) fold. The difference in the amount of the biomarker is preferably statistically significant. By “statistically significant”, it is meant that the alteration is greater than what might be expected to happen by chance alone.

As illustrated in FIG. 4, the present inventors observed that some of the “survival” biomarkers described herein increase in abundance in patients that did not survive as compared to patients that made a full recovery from a systemic inflammatory condition, and as compared to healthy individuals. These differences in marker abundance can be used to predict whether a patient is likely to survive a systemic inflammatory condition, and can thus be used to determine whether a patient is suitable for discharge from medical care. For example, the biomarkers PKHD1 and NECAB1 increased in abundance in SIRS patients that did not survive as compared to SIRS patients that made a full recovery from a systemic inflammatory condition, and as compared to healthy individuals. The biomarkers PKD1 and NECAB2 also increased in abundance in abdominal sepsis patients that did not survive as compared to abdominal sepsis patients that made a full recovery from a systemic inflammatory condition, and as compared to healthy individuals.

As illustrated in FIG. 4, the present inventors observed that some of the “survival” biomarkers described herein increase in abundance in patients that made a full recovery from a systemic inflammatory condition as compared to patients that did not survive. These differences in marker abundance can be used to predict whether a patient is likely to survive a systemic inflammatory condition, and can thus be used to determine whether a patient is suitable for discharge from medical care. For example, the biomarker LILRB5 increased in abundance in pulmonary sepsis patients that made a full recovery from pulmonary sepsis as compared to pulmonary sepsis patients that did not survive.

By comparing the amount of markers quantified in a sample obtained from a patient to the amount of markers quantified for a reference value (such as that obtained from a healthy individual (or a population of healthy individuals), an individual (or population of individuals) that has a (good) prognosis of recovery (or survival) from a systemic inflammatory condition, and/or an individual (or population of individuals) that has a prognosis of non-recovery (or non-survival) from a systemic inflammatory condition), it is possible to determine whether a patient is suitable for discharge from medical care. The method permits classification of the patient as belonging to or not belonging to the reference population (ie. by determining whether the amounts of marker quantified in the patient are statistically similar to the reference population or statistically deviate from the reference population). Hence, classification of the patient's marker profile (ie. the overall pattern of change observed for the markers quantified) as corresponding to the profile derived from a particular reference population is predictive that the individual falls (or does not fall) within the reference population.

In one embodiment, a patient may be identified as being suitable for discharge from medical care, when the amount of the one or more biomarker is statistically similar to the amount determined for the corresponding reference value representative of an individual (or a population of individuals) that has a (good) prognosis of recovery (or survival) from a systemic inflammatory condition. In one embodiment, a patient may be identified as being suitable for discharge from medical care, when the amount of the one or more biomarker is statistically similar to the amount determined for the corresponding reference value representative of a healthy individual (or a population of healthy individuals). In one embodiment, a patient may be identified as being unsuitable for discharge from medical care when the amount of the one or more biomarker is statistically similar to the amount determined for the corresponding reference value representative of an individual (or a population of individuals) having a prognosis of non-recovery (or non-survival) from a systemic inflammatory condition.

In one embodiment, a patient may be identified as being unsuitable for discharge from medical care, when the amount of the one or more biomarker statistically deviates from the amount determined for the corresponding reference value representative of an individual (or a population of individuals) that has a (good) prognosis of recovery (or survival) from a systemic inflammatory condition. In one embodiment, a patient may be identified as being unsuitable for discharge from medical care, when the amount of the one or more biomarker statistically deviates from the amount determined for the corresponding reference value representative of a healthy individual (or a population of healthy individuals). In one embodiment, a patient may be identified as being suitable for discharge from medical care when the amount of the one or more biomarker statistically deviates from the amount determined for the corresponding reference value representative of an individual (or a population of individuals) that has a prognosis of non-recovery (or non-survival) from a systemic inflammatory condition.

All embodiments described above (in the context of the diagnostic methods) for classifying a patient based on their marker profile apply equally to the method for determining whether a patient is suitable for discharge from medical care. This includes all embodiments for determining whether the marker profile of the patient is “statistically similar to” or “statistically deviates from” the marker profiles observed for the corresponding reference values, and all embodiments relating to the % increase or % decrease or fold change observed in the markers as compared to the corresponding reference value.

The reference value may be as defined above for the diagnostic methods described herein. In one embodiment, the reference value is representative of a healthy individual (or a population of healthy individuals). In one embodiment, the reference value may be representative of an individual (or population of individuals) that has a (good) prognosis of recovery (or survival) from a systemic inflammatory condition. In one embodiment, the reference value may be representative of an individual (or population of individuals) that has a prognosis of non-recovery (or non-survival) from a systemic inflammatory condition).

The reference value that is representative of an individual (or population of individuals) having a (good) prognosis of recovery (or survival) from a systemic inflammatory condition is determined by quantifying the amount of the one or more biomarker in a sample obtained from an individual (or population of individuals) having a systemic inflammatory condition, wherein the individual (or population of individuals) goes on to make a full recovery from the systemic inflammatory condition. The sample may be obtained at least 1 hour, 2 hours, 4 hours, 6 hours, 8 hours, 12 hours, 36 hours, 48 hours, 72 hours, 96 hours, or 120 hours, after the individual presents with one or more clinical symptoms of a systemic inflammatory condition, or is admitted to a medical care facility, For example, the sample may be obtained at least 120 hours after the individual (or population of individuals) presents with one or more clinical symptoms of a systemic inflammatory condition, or is admitted to a medical care facility.

The reference value that is representative of an individual (or population of individuals) having a prognosis of non-recovery (or non-survival) from a systemic inflammatory condition, or a poor prognosis of recovery (or survival) is determined by quantifying the amount of biomarker in a sample obtained from an individual (or population of individuals) having a systemic inflammatory condition, wherein the individual (or population of individuals) does not recover from the systemic inflammatory condition. The sample may be obtained at least 1 hour, 2 hours, 4 hours, 6 hours, 8 hours, 12 hours, 36 hours, 48 hours, 72 hours, 96 hours, or 120 hours, after the individual presents with one or more clinical symptoms of a systemic inflammatory condition, or is admitted to a medical care facility. For example, the sample may be obtained at least 120 hours after the individual (or population of individuals) presents with one or more clinical symptoms of a systemic inflammatory condition, or is admitted to a medical care facility.

The present inventors observed that some of the ‘survival’ biomarkers described herein increase in abundance in non-survivors as compared to healthy individuals. Detection of increased levels of these biomarkers in a patient as compared to the levels detected for healthy individuals can thus be used to determine whether a patient is suitable for discharge from medical care.

In one embodiment, when the reference value is representative of a healthy individual (or a population of healthy individuals), an increase in the one or more biomarker in the sample obtained from the patient relative to the corresponding reference value representative of a healthy individual, indicates that the patient is not suitable for discharge from medical care. Likewise, no increase in the one or more biomarker in the sample obtained from the patient relative to the corresponding reference value representative of a healthy individual, indicates that the patient is suitable for discharge from medical care.

For some of the ‘survival’ biomarkers identified by the present inventors, increased levels of these markers were also observed in patients that recovered from a systemic inflammatory condition as compared to healthy individuals, although much bigger increases were observed for these biomarkers in the patients that did not survive. The accuracy of determining whether a patient is suitable for discharge from medical care can thus be improved by looking for a “minimum” fold change in the levels of the one or more biomarkers as compared to the corresponding reference value that is representative of a healthy individual.

For example, an increase of at least 2.5 (eg. at least 2.6, at least 2.7, at least 2.8, at least 2.9, at least 3, at least 3.1, at least 3.2, at least 3.3, at least 3.4, at least 3.5, at least 3.6, at least 3.7, at least 3.8, at least 3.9, at least 4) fold in NECAB1 in the sample obtained from the patient relative to the corresponding reference value representative of a healthy individual, indicates that the patient is not suitable for discharge from medical care. No increase or an increase of less than 1.5 (eg. less than 1.4, less than 1.3, less than 1.2, less than 1.1, less than 1) fold in NECAB1 in the sample obtained from the patient relative to the corresponding reference value representative of a healthy individual, indicates that the patient is suitable for discharge from medical care. In one embodiment, the patient is undergoing (or has undergone) treatment for SIRS. In one embodiment, the sample is obtained from the patient at least 72 hours (eg. at least 96 hours, at least 120 hours) after the patient presents with one or more clinical symptoms of a systemic inflammatory condition, or is admitted to a medical care facility.

For example, an increase of at least 3.5 (eg. at least 3.6, at least 3.7) fold in NECAB2 in the sample obtained from the patient relative to the corresponding reference value representative of a healthy individual, indicates that the patient is not suitable for discharge from medical care. No increase or an increase of less than 2.5 (eg. less than 2.4, less than 2.3, less than 2.2, less than 2.1, less than 2, less than 1.5, less than 1.4, less than 1.3, less than 1.2, less than 1.1, less than 1) fold in NECAB2 in the sample obtained from the patient relative to the corresponding reference value representative of a healthy individual, indicates that the patient is suitable for discharge from medical care. In one embodiment, the patient is undergoing (or has undergone) treatment for sepsis. In one embodiment, the patient is undergoing (or has undergone) treatment for abdominal sepsis and/or pulmonary sepsis. In one embodiment, the sample is obtained from the patient at least 48 hours (eg. at least 72 hours, at least 96 hours, at least 120 hours) after the patient presents with one or more clinical symptoms of a systemic inflammatory condition, or is admitted to a medical care facility.

For example, an increase of at least 1.5 (eg. at least 1.6, at least 1.7, at least 1.8, at least 1.9, at least 2, at least 2.1, at least 2.2) fold in PKD1 in the sample obtained from the patient relative to the corresponding reference value representative of a healthy individual, indicates that the patient is not suitable for discharge from medical care. No increase or an increase of less than 1.6 (eg. less than 1.5, less than 1.4, less than 1.3, less than 1.2, less than 1.1, less than 1) fold in PKD1 in the sample obtained from the patient relative to the corresponding reference value representative of a healthy individual, indicates that the patient is suitable for discharge from medical care. In one embodiment, the patient is undergoing (or has undergone) treatment for abdominal sepsis and/or SIRS. In one embodiment, the sample is obtained from the patient at least 72 hours (eg. at least 96 hours, at least 120 hours) after the patient presents with one or more clinical symptoms of a systemic inflammatory condition, or is admitted to a medical care facility.

For example, an increase of at least 2.5 (eg. at least 3, at least 3.5, at least 4, at least 4.5, at least 5, at least 5.1, at least 5.2, or at least 5.3) fold in PKHD1 in the sample obtained from the patient relative to the corresponding reference value representative of a healthy individual, indicates that the patient is not suitable for discharge from medical care. No increase or an increase of less than 1.5 (eg. less than 1.4, less than 1.3, less than 1.2, less than 1.1, less than 1) fold in PKHD1 in the sample obtained from the patient relative to the corresponding reference value representative of a healthy individual, indicates that the patient is suitable for discharge from medical care. In one embodiment, the patient is undergoing (or has undergone) treatment for SIRS. In one embodiment, the sample is obtained from the patient at least 72 hours (eg. at least 96 hours, at least 120 hours) after the patient presents with one or more clinical symptoms of a systemic inflammatory condition, or is admitted to a medical care facility.

For example, an increase of at least 6.3 (eg. at least 6.4, at least 6.5, at least 6.6, at least 6.7, at least 6.8, at least 6.9, at least 7) fold in LILRB4 in the sample obtained from the patient relative to the corresponding reference value representative of a healthy individual, indicates that the patient is not suitable for discharge from medical care. No increase or an increase of less 5.5 (eg. less than 5.4, less than 5.3, less than 5.2, less than 5.1, less than 5) fold in LILRB4 in the sample obtained from the patient relative to the corresponding reference value representative of a healthy individual, indicates that the patient is suitable for discharge from medical care. In one embodiment, the patient is undergoing (or has undergone) treatment for SIRS. In one embodiment, the sample is obtained from the patient at least 72 hours (eg. at least 96 hours, at least 120 hours) after the patient presents with one or more clinical symptoms of a systemic inflammatory condition, or is admitted to a medical care facility.

For example, an increase of at least 7 (eg. at least 7.1, at least 7.2, at least 7.3 at least 7.4, at least 7.5, at least 7.6, at least 7.7, at least 7.8, at least 7.9, at least 8) fold in LILRB5 in the sample obtained from the patient relative to the corresponding reference value representative of a healthy individual, indicates that the patient is not suitable for discharge from medical care. No increase or an increase of less than 4.5 (eg. less than 5, less than 5.5, less than 6, less than 6.5, less than 7) fold in LILRB5 in the sample obtained from the patient relative to the corresponding reference value representative of a healthy individual, indicates that the patient is suitable for discharge from medical care. In one embodiment, the patient is undergoing (or has undergone) treatment for SIRS. In one embodiment, the sample is obtained from the patient at least 72 hours (eg. at least 96 hours, at least 120 hours) after the patient presents with one or more clinical symptoms of a systemic inflammatory condition, or is admitted to a medical care facility.

As described herein, LILRB5 was also observed to increase in abundance in patients that made a full recovery from pulmonary sepsis compared to patients that did not survive. Thus, in one example, an increase of at least 2.5 (eg. at least 3, at least 3.5, at least 4, at least 4.5, at least 5) fold in LILRB5 in the sample obtained from the patient relative to the corresponding reference value representative of a healthy individual, indicates that the patient is suitable for discharge from medical care. No increase or an increase of less than 4.5 (eg. less than 4, less than 3.5, less than 3, less than 2.5, less than 2) fold in LILRB5 in the sample obtained from the patient relative to the corresponding reference value representative of a healthy individual, indicates that the patient is not suitable for discharge from medical care. In one embodiment, the patient is undergoing (or has undergone) treatment for pulmonary sepsis. In one embodiment, the sample is obtained from the patient at least 72 hours (eg. at least 96 hours or at least 120 hours) after the patient presents with one or more clinical symptoms of a systemic inflammatory condition, or is admitted to a medical care facility.

As described herein, the present inventors observed that the levels of some of the one or more survival biomarkers were elevated in patients that did not recover from (or survive) a systemic inflammatory condition as compared to patients that made a full recovery. Detection of increased levels of these biomarkers in a patient as compared to the levels detected for patients that recovered from (or survived) a systemic inflammatory condition can thus be used to determine whether a patient is suitable for discharge from medical care.

Thus, in one embodiment, when the reference value is representative of an individual (or population of individuals) having a (good) prognosis of recovery (or survival) from a systemic inflammatory condition, an increase in the one or more biomarker in the sample obtained from the patient relative to the corresponding reference value, indicates that the patient is not suitable for discharge from medical care. Likewise, no increase in the one or more biomarker in the sample obtained from the patient relative to the corresponding reference value, indicates that the patient is suitable for discharge from medical care.

In one embodiment, the patient may be identified as being unsuitable for discharge from medical care, when the one or more biomarker increases by at least 1 (e.g. at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value.

As described herein, the present inventors observed that the levels of some of the one or more survival biomarkers were elevated in patients that made a full recovery from a systemic inflammatory condition as compared to patients that did not recover from (or survive). Detection of increased levels of these biomarkers in a patient as compared to the levels detected for patients that recovered from (or survived) a systemic inflammatory condition can thus be used to determine whether a patient is suitable for discharge from medical care.

Thus, in one embodiment, when the reference value is representative of an individual (or population of individuals) having a (good) prognosis of recovery (or survival) from a systemic inflammatory condition, an increase in the one or more biomarker in the sample obtained from the patient relative to the corresponding reference value, indicates that the patient is suitable for discharge from medical care. Likewise, no increase in the one or more biomarker in the sample obtained from the patient relative to the corresponding reference value, indicates that the patient is not suitable for discharge from medical care.

In one embodiment, the patient may be identified as being suitable for discharge from medical care, when the one or more biomarker increases by at least 1 (e.g. at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value.

As described above for the diagnostic methods described herein, the method of the invention may involve the use of multiple separate reference values. For example, the method may involve the use of one or more (eg. two or more, or all three) reference values that are representative of an individual (or population of individuals) having a (good) prognosis of recovery (or survival) from a systemic inflammatory condition, an individual (or population of individuals) having a prognosis of non-recovery (or non-survival) (or a poor prognosis of recovery) from a systemic inflammatory condition; and a healthy individual (or a population of healthy individuals).

In a related aspect, the present invention also provides the use of one or more biomarker selected from: NECAB1, NECAB2, PKD1, PKHD1, LILRB4 and LILRB5 for determining whether a patient is suitable for discharge from medical care.

In one embodiment, the present invention provides the use of one or more biomarker selected from: PKHD1, PKDI, NECAB1, LILRB4, and LILRB5 for determining whether a patient is suitable for discharge from medical care. The patient may be undergoing (or has undergone) treatment for SIRS. In one embodiment, the one or more biomarker may be selected from: PKHD1 and NECAB1. For example, the one or more biomarker may comprise the combination of PKHD1 and NECAB1. For example, the one or more biomarker is PKHD1. For example, the one or more biomarker is NECAB1.

In one embodiment, the present invention provides the use of one or more biomarker selected from the group consisting of NECAB2, LILRB5, PKHD1 and PKD1 for determining whether a patient is suitable for discharge from medical care. The patient may be undergoing (or has undergone) treatment for sepsis (such as abdominal sepsis and/or pulmonary sepsis).

In one embodiment, the present invention provides the use of one or more biomarker selected from: NECAB2 and PKD1, for determining whether a patient is suitable for discharge from medical care. The patient may be undergoing (or has undergone) treatment for abdominal sepsis. For example, the one or more biomarker may comprise the combination of biomarkers NECAB2 and PKD1. For example, the one or more biomarker is NECAB2. For example, the one or more biomarker is PKD1.

In one embodiment, the present invention provides the use of one or more biomarker selected from: PKHD1 and LILRB5, for determining whether a patient is suitable for discharge from medical care. The patient may be undergoing (or has undergone) treatment for pulmonary sepsis. For example, the one or more biomarker may comprise the combination of biomarkers PKHD1 and LILRB5. For example, the one or more biomarker is PKHD1. For example, the one or more biomarker is LILRB5.

All embodiments described above for the method of determining whether a patient is suitable for discharge from medical care apply equally to the ‘use’ of the invention described herein. This includes all embodiments relating to the terms “systemic inflammatory condition”, “patient”, “sample”, and “the one or more biomarker”.

Systemic inflammatory conditions such as SIRS and sepsis can lead to the development of multiple organ failure in patients. Early detection of organ failure in patients may improve the chances of survival in patients having a systemic inflammatory condition.

When investigating the biomarkers associated with systemic inflammatory conditions, the present inventors surprisingly observed that various organ specific biomarkers are present in high levels in peripheral blood leukocytes (PBLs) obtained from patients having systemic inflammatory conditions that did not survive. These biomarkers include the brain specific markers NECAB1 and NECAB2, and the kidney specific markers PKHD1 and PKD1. The presence of these markers in peripheral blood leukocytes indicates that the organ is damaged. Detection of these markers in samples obtained from patients therefore provides a way of diagnosing organ damage in the patient.

The present invention provides a method for diagnosing organ damage in a patient, comprising:

(i) determining the presence (and/or amount) of one or more biomarker selected from: NECAB1, NECAB2, PKHD1, and PKD1, in a sample obtained from a patient,
(ii) comparing the presence (and/or amount) of the one or more biomarker determined in said sample in (i) to a corresponding reference value; and thereby determining the patient has or is at risk of developing organ damage.

All embodiments described above for the method of determining whether a patient is suitable for discharge from medical care apply equally to the method for diagnosing organ damage in a patient. This includes all embodiments relating to the “patient”, “sample”, “reference value”, and the steps for “determining the presence and/or amount of the one or more biomarker” and the “comparison” for making a conclusion about the diseases state of the patient.

The term “organ damage” refers to the condition where an organ has been injured such that it does not perform its expected function. In one embodiment the organ damage is one or more of: brain damage or kidney damage.

The “patient” is as described above for the “method of determining whether a patient is suitable for discharge from medical care”. The method of the invention for diagnosing organ damage is not only applicable to such patients, but may also be used to diagnose organ damage in patients having a disease or condition other than a systemic inflammatory condition. The patient may thus be an individual having any disease, condition or injury which may result in organ damage.

The “one or more biomarker” of the invention is as described above for the “method of determining whether a patient is suitable for discharge from medical care”. In one embodiment, the one or more biomarker is selected from the group consisting of: NECAB1, NECAB2, PKHD1, and PKD1.

In one embodiment, the one or more biomarker is selected from NECAB1 and/or NECAB2. These biomarkers are specific for indicating the presence of brain damage in a patient. Thus when the one or more biomarker is selected from NECAB1 and/or NECAB2, the method is for diagnosing brain damage in a patient. By performing steps (i) and (ii) of the method described herein, the method can be used to determine whether a patient has or is at risk of developing brain damage.

In one embodiment, the one or more biomarker is selected from PKHD1 and/or PKD1. These biomarkers are specific for indicating the presence of kidney damage in a patient. Thus when the one or more biomarker is selected from PKHD1 and/or PKD1, the method is for diagnosing kidney damage in a patient. By performing steps (i) and (ii) of the method described herein, the method can be used to determine whether a patient has or is at risk of developing kidney damage.

Each of the biomarkers may be used alone, or in combination with any of the biomarkers described herein in the method of the invention. For example, any combination of 1 or more, 2 or more, 3 or more, or all 4 or more of the biomarkers may be used in the method of the invention.

In one embodiment, any combination of 1 or more (eg. 2 or more, 3 or more, or all 4) of the biomarkers selected from the group consisting of: NECAB1, NECAB2, PKD1, and PKHD1, may be used to diagnose organ damage in a patient.

The ‘quantification’ and ‘comparison’ steps of the method, and the “reference value” used in the ‘comparison’ step are as described above for the method for determining whether a patient is suitable for discharge from medical care. This includes all embodiments described for classification of a patient based on their marker profile.

As described herein, the present inventors observed that the organ specific biomarkers described herein each increase in abundance in samples obtained from patients having a systemic inflammatory condition as compared to healthy individuals. However, much higher levels of the organ specific biomarkers were observed in patients that did not survive the systemic inflammatory condition as compared to those patients that recovered.

In one embodiment, the reference value is representative of a healthy individual (or a population of healthy individuals). The patient may be diagnosed as having organ damage or being at risk of developing organ damage when an increase is observed in the one or more biomarker in the sample obtained from the patient relative to the corresponding reference value representative of a healthy individual. More accurate diagnosis can be performed by looking for a minimum fold increase in the one or more biomarker in the patient. The minimum fold change values in the biomarkers NECAB1, NECAB2, PKD1 and PKHD1 are as defined above for the method for determining whether a patient is suitable for discharge from medical care.

As described herein, the present inventors observed that the levels of the organ specific biomarkers were elevated in patients that did not recover from (or survive) a systemic inflammatory condition as compared to patients that made a full recovery. The patients that did not survive are likely to have a higher risk of organ failure as compared to patients that made a full recovery. Detection of increased levels of these biomarkers in a patient as compared to the levels detected for patients that recovered from (or survived) a systemic inflammatory condition can thus be used to determine whether a patient has organ damage.

Thus, in one embodiment, when the reference value is representative of an individual (or population of individuals) having a (good) prognosis of recovery (or survival) from a systemic inflammatory condition, an increase in the one or more biomarker in the sample obtained from the patient relative to the corresponding reference value, indicates that the patient has or is at risk of developing organ damage.

In one embodiment, the patient may be diagnosed as having or being at risk of developing organ damage, when the one or more biomarker increases by at least 1 (e.g. at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15 fold, at least 20 fold, at least 30 fold, at least 40 fold, at least 50) fold in the sample obtained from the patient relative to the corresponding reference value.

As described above for the method for determining whether a patient is suitable for discharge from medical care, multiple separate reference values may be used in the method for diagnosing organ damage. All combinations of reference values described above apply equally to the method for diagnosing organ damage.

In a related aspect, the present invention also provides the use of one or more of: NECAB1, NECAB2, PKHD1, and PKD1, as a biomarker for organ damage. In one embodiment, the use is of the one or more biomarker for diagnosis of organ damage in a patient.

In one embodiment, the use is of one or more biomarker selected from the group consisting of: NECAB1 and NECAB2, for diagnosis of brain damage in a patient. In one embodiment, the use is of one or more biomarker selected from the group consisting of: PKHD1 and PKD1, for diagnosis of kidney damage in a patient.

All embodiments described above for the method of diagnosing organ damage in a patient apply equally to the ‘use’ of the invention described herein. This includes all embodiments relating to the “patient”, “sample”, and “the one or more biomarker”.

### Treatment

The methods described herein for diagnosis and/or monitoring of a systemic inflammatory condition in a patient may in certain embodiments also be applied to determine whether the patient is or is not in need of a therapeutic or prophylactic treatment of the systemic inflammatory condition. For example, a treatment may be indicated where the methods allow for a conclusion that the patient has or is at risk of having a systemic inflammatory condition, has a poor prognosis for the systemic inflammatory condition, displays a detrimental development of the condition, or has organ damage. Without limitation, a patient with the systemic inflammatory condition upon admission to or during stay in a medical care centre such as ICU may be tested as described herein for the necessity of continuing the treatment of the condition, and may be discharged when such treatment is no longer needed or is needed only to a given limited extent.

In a further embodiment, any of the methods described herein may further comprise treating a systemic inflammatory condition in a patient. In one embodiment, any of the methods described herein may comprise, responsive to the diagnosis of a systemic inflammatory condition in the patient, administering to the patient a therapy for a systemic inflammatory condition. For example, the therapy may be for SIRS and/or for sepsis. The methods of the invention may therefore be for treating or preventing one or more symptoms of a systemic inflammatory condition.

For example, any of the methods described herein for diagnosis of SIRS (including the method for diagnosing SIRS in a patient, and the method for distinguishing between sepsis and SIRS in a patient) may further comprise, responsive to the diagnosis of SIRS, administering to the patient a therapy for SIRS. These methods may be for treating or preventing one or more symptoms of SIRS in a patient.

The “therapy for SIRS” may include: organ support with oxygen, mechanical ventilation, circulatory support with fluid resuscitation, vasodilators, inotropes or vasopressors, renal replacement therapy.

In certain embodiments, the administering of a therapy for SIRS may comprise administering one such therapy to the patient. In certain embodiments, the administering of a therapy for SIRS may comprise administering a combination of two or more such therapies to the patient.

For example, any of the methods described herein for diagnosis of sepsis (including the method for diagnosing sepsis in a patient, the method for distinguishing between sepsis and SIRS in a patient, the method for diagnosing abdominal sepsis in a patient, the method for diagnosing pulmonary sepsis in a patient, and the method for distinguishing between abdominal sepsis and pulmonary sepsis in a patient) may further comprise, responsive to the diagnosis of sepsis, administering to the patient a therapy for sepsis. These methods may be for treating or preventing one or more symptoms of sepsis in a patient.

The “therapy for sepsis” may include anti-microbial agents (such as anti-bacterial agents e.g. antibiotics), analgesics, antipyretics, anti-inflammatory drugs (such as non-steroidal anti-inflammatory drugs), fluid resuscitation, and oxygen therapy. It may also include organ support with oxygen, mechanical ventilation, circulatory support with inotropes or vasopressors, renal replacement therapy.

In certain embodiments, the administering of a therapy for sepsis may comprise administering one such therapy to the patient. In certain embodiments, the administering of a therapy for sepsis may comprise administering a combination of two or more such therapies to the patient.

In one embodiment, the method for distinguishing between sepsis and SIRS in a patient may further comprise, responsive to the diagnosis of sepsis and/or SIRS in the patient, administering to the patient a therapy for sepsis and/or SIRS. For example, the therapy may be for SIRS as described herein. Alternatively, the therapy may be for sepsis (including abdominal sepsis and pulmonary sepsis) as described herein. The methods of the invention may therefore be for treating or preventing one or more symptoms of sepsis and/or SIRS.

In one embodiment, any of the methods described herein for diagnosis of organ damage may further comprise, responsive to the diagnosis of organ damage, administering to the patient a therapy for organ damage.

### Oligonucleotide Probes and Amplification Primers

Any appropriate detection means can be used to detect or quantify the one or more biomarker in the methods and uses of the invention, as described herein.

Typically when the one or more biomarker of the invention is a nucleic acid, the presence of the one or more biomarker may be detected, and/or the amount of the one or more biomarker determined using an oligonucleotide probe. The methods and uses described herein may therefore use any one or more oligonucleotide probe as defined herein to detect and/or quantify the one or more biomarker of the invention. The oligonucleotide probes may be bound to a solid surface (such as a microarray). Alternatively oligonucleotide probes may be used in quantitative real-time PCR to detect amplified target sequence from the one or more biomarker.

An oligonucleotide probe of the invention may have at least 80% sequence identity to the one or more biomarker of the invention, or a target region within said biomarker, measured over any appropriate length of sequence. Typically the % sequence identity is determined over a length of contiguous nucleic acid residues. An oligonucleotide probe of the invention may, for example, have at least 80% sequence identity to the one or more biomarker of the invention, or target region thereof, measured over at least 10, at least 20, at least 30, at least 40, at least 50, at least 60, at least 70, at least 80, at least 90, or more nucleic acid residues, up to the oligonucleotide probe having at least 80% sequence identity with the one or more biomarker of the invention, or target region thereof, over the entire length of the oligonucleotide probe.

An oligonucleotide probe of the invention may be complementary to the one or more nucleic acid biomarker of the invention, or a target region thereof. Typically the oligonucleotide probe of the invention is complementary over a length of contiguous nucleic acid residues. An oligonucleotide probe of the invention may, for example, be complementary to the one or more biomarker of the invention, or target region thereof, measured over at least 10, at least 20, at least 30, at least 40, at least 50, at least 60, at least 70, at least 80, at least 90, or more nucleic acid residues, up to the oligonucleotide probe having being complementary to the one or more biomarker of the invention, or target region thereof, over the entire length of the oligonucleotide probe.

An oligonucleotide probe of the invention may be complementary to a variant of the one or more biomarker of the invention, or a variant of a target region of said biomarker. Typically the oligonucleotide probe is complementary to a variant having at least 80% sequence identity to the one or more biomarker of the invention, or a variant having at least 80% sequence identity to the target region of said biomarker. The % sequence identity of the variant to the one or more biomarker of the invention, or a variant of a target region of said biomarker may be calculated over any appropriate length of sequence in the one or more biomarker, as described herein.

As used herein, a “sequence identity of at least 80%” includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M—A New Algorithm for Multiple Alignment of Highly Divergent Sequences, 20 (9) Bioinformatics: 1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992.

Variants of the specific sequences described herein may alternatively be defined by reciting the number of nucleotides that differ between the variant sequences and the specific reference sequences provided above. Thus, in one embodiment, the sequence may comprise (or consist of) a nucleotide sequence that differs from the specific sequences provided above at no more than 2 nucleotide positions, for example at no more than 1 nucleotide position. Conservative substitutions are preferred. The term variants as defined herein also encompasses splice variants.

An oligonucleotide probe of the invention may be at least 30, at least 40, at least 50, at least 60, at least 70, at least 80, at least 90, at least 100, or more nucleotides in length. In a preferred embodiment, the oligonucleotide probe is 40 to 100 nucleotides in length, more preferably 50 to 100 nucleotides in length, even more preferably 50 to 80 nucleotides in length and most preferably 50 to 70 nucleotides in length. Such oligonucleotide probes are suitable for use in use in microarray analysis when bound to a solid surface. In one embodiment, the oligonucleotide probe is designed for detection of the one or more biomarker by microarray analysis.

Oligonucleotide probes may also be designed for detection of the one or more biomarker by quantitative PCR (or real-time PCR). The oligonucleotide probe may be 5-30 nucleotides long, such as at least 6, 7, 8, 9 or 10 nucleotides long. The oligonucleotide probe may be up to 25 nucleotides long, such as up to 20, 18, 16, 15, 14, 13, 12, 11 or 10 nucleotides long. The oligonucleotide probe may be 10-25 nucleotides long, such as 10-20 nucleotides long or 10-15 nucleotides long, and may be preferably about 10 nucleotides long. In this regard, the use of short probes enables faster annealing to the target nucleic acid.

The target nucleotide sequence to which the oligonucleotide probe hybridises within the amplification product may be at least 5, 6, 7, 8, 9 or 10 nucleotides long. The target sequence for the probe may be up to 30 nucleotides long, such as up to 25, 20, 18, 16, 15, 14, 13, 12, or 11 nucleotides long. The probe target sequence may be 10-25 nucleotides long or 10-15 nucleotides long, and may be preferably about 10 nucleotides long.

The probes of the invention are typically designed to hybridise to their target nucleic acid sequence present in the one or more biomarker of the invention.

A probe may comprise or be complementary to a nucleic acid sequence within a target nucleic acid sequence from the one or more biomarker of the invention, or to a nucleic acid sequence having at least 80% identity to said target nucleic acid sequence. Any suitable probe which comprises or is complementary (as defined herein) to a nucleic acid sequence within a target nucleic acid sequence of one or more biomarker of the invention may be used.

In embodiments wherein the one or more biomarker is ADM, a target nucleic acid sequence may comprise bases 751 to 1590 of SEQ ID NO: 1, and a probe typically comprises or is complementary to a nucleic acid sequence having at least 80% sequence identity to a nucleic acid sequence from this target sequence.

In embodiments wherein the one or more biomarker is CD177, a target nucleic acid sequence may comprise bases 1351 to 2220 of SEQ ID NO: 2, and a probe typically comprises or is complementary to a nucleic acid sequence having at least 80% sequence identity to a nucleic acid sequence from this target sequence.

In embodiments wherein the one or more biomarker is FAM20A, a target nucleic acid sequence may comprise bases 1331 to 3700 of SEQ ID NO: 3, and a probe typically comprises or is complementary to a nucleic acid sequence having at least 80% sequence identity to a nucleic acid sequence from this target sequence. For example, a target nucleic acid sequence may comprise bases 1460 to 1531 of SEQ ID NO: 3 or bases 1486 to 1551 of SEQ ID NO: 3, and a probe typically comprises or is complementary to a nucleic acid sequence having at least 80% sequence identity to a nucleic acid sequence from this target sequence.

In embodiments wherein the one or more biomarker is IL10, a target nucleic acid sequence may comprise bases 61 to 1320 of SEQ ID NO: 4, and a probe typically comprises or is complementary to a nucleic acid sequence having at least 80% sequence identity to a nucleic acid sequence from this target sequence.

In embodiments wherein the one or more biomarker is METTL7B, a target nucleic acid sequence may comprise bases 581 to 1340 of SEQ ID NO: 5, and a probe typically comprises or is complementary to a nucleic acid sequence having at least 80% sequence identity to a nucleic acid sequence from this target sequence.

In embodiments wherein the one or more biomarker is MMP9, a target nucleic acid sequence may comprise bases 1511 to 2330 of SEQ ID NO: 6, and a probe typically comprises or is complementary to a nucleic acid sequence having at least 80% sequence identity to a nucleic acid sequence from this target sequence.

In embodiments wherein the one or more biomarker is RETN, a target nucleic acid sequence may comprise bases 81 to 478 of SEQ ID NO: 7, and a probe typically comprises or is complementary to a nucleic acid sequence having at least 80% sequence identity to a nucleic acid sequence from this target sequence.

In embodiments wherein the one or more biomarker is TDRD9, a target nucleic acid sequence may comprise bases 3711 to 4400 of SEQ ID NO: 8, and a probe typically comprises or is complementary to a nucleic acid sequence having at least 80% sequence identity to a nucleic acid sequence from this target sequence.

In embodiments wherein the one or more biomarker is ITGA7, a target nucleic acid sequence may comprise bases 3181 to 4080 of SEQ ID NO: 9, and a probe typically comprises or is complementary to a nucleic acid sequence having at least 80% sequence identity to a nucleic acid sequence from this target sequence.

In embodiments wherein the one or more biomarker is BMX, a target nucleic acid sequence may comprise bases 1651 to 2430 of SEQ ID NO: 10, and a probe typically comprises or is complementary to a nucleic acid sequence having at least 80% sequence identity to a nucleic acid sequence from this target sequence.

In embodiments wherein the one or more biomarker is HP, a target nucleic acid sequence may comprise bases 821 to 1430 of SEQ ID NO: 11, and a probe typically comprises or is complementary to a nucleic acid sequence having at least 80% sequence identity to a nucleic acid sequence from this target sequence.

In embodiments wherein the one or more biomarker is IGFBP2, a target nucleic acid sequence may comprise bases 651 to 1430 of SEQ ID NO: 12, and a probe typically comprises or is complementary to a nucleic acid sequence having at least 80% sequence identity to a nucleic acid sequence from this target sequence.

In embodiments wherein the one or more biomarker is ALPL, a target nucleic acid sequence may comprise bases 1441 to 2520 of SEQ ID NO: 13, and a probe typically comprises or is complementary to a nucleic acid sequence having at least 80% sequence identity to a nucleic acid sequence from this target sequence.

In embodiments wherein the one or more biomarker is DACH1, a target nucleic acid sequence may comprise bases 2341 to 4990 of SEQ ID NO: 14, and a probe typically comprises or is complementary to a nucleic acid sequence having at least 80% sequence identity to a nucleic acid sequence from this target sequence.

In embodiments wherein the one or more biomarker is IL1R1, a target nucleic acid sequence may comprise bases 1551 to 4410 of SEQ ID NO: 15, and a probe typically comprises or is complementary to a nucleic acid sequence having at least 80% sequence identity to a nucleic acid sequence from this target sequence.

In embodiments wherein the one or more biomarker is OLAH, a target nucleic acid sequence may comprise bases 781 to 1480 of SEQ ID NO: 16, and a probe typically comprises or is complementary to a nucleic acid sequence having at least 80% sequence identity to a nucleic acid sequence from this target sequence. For example, a target nucleic acid sequence may comprise bases 901 to 960 of SEQ ID NO: 16, and a probe typically comprises or is complementary to a nucleic acid sequence having at least 80% sequence identity to a nucleic acid sequence from this target sequence. In an alternative embodiment, a target nucleic acid sequence may comprise bases 632 to 697 of SEQ ID NO: 16, and a probe typically comprises or is complementary to a nucleic acid sequence having at least 80% sequence identity to a nucleic acid sequence from this target sequence.

In embodiments wherein the one or more biomarker is IL1R2, a target nucleic acid sequence may comprise bases 681 to 1310 of SEQ ID NO: 17, and a probe typically comprises or is complementary to a nucleic acid sequence having at least 80% sequence identity to a nucleic acid sequence from this target sequence.

In embodiments wherein the one or more biomarker is CYP19A1, a target nucleic acid sequence may comprise bases 441 to 4520 of SEQ ID NO: 18, and a probe typically comprises or is complementary to a nucleic acid sequence having at least 80% sequence identity to a nucleic acid sequence from this target sequence.

In embodiments wherein the one or more biomarker is MMP8, a target nucleic acid sequence may comprise bases 1621 to 2900 of SEQ ID NO: 19, and a probe typically comprises or is complementary to a nucleic acid sequence having at least 80% sequence identity to a nucleic acid sequence from this target sequence.

In embodiments wherein the one or more biomarker is TGFA, a target nucleic acid sequence may comprise bases 3321 to 4110 of SEQ ID NO: 20, and a probe typically comprises or is complementary to a nucleic acid sequence having at least 80% sequence identity to a nucleic acid sequence from this target sequence.

In embodiments wherein the one or more biomarker is VSTM1, a target nucleic acid sequence may comprise bases 271 to 990 of SEQ ID NO: 21, and a probe typically comprises or is complementary to a nucleic acid sequence having at least 80% sequence identity to a nucleic acid sequence from this target sequence.

In embodiments wherein the one or more biomarker is FCER1A, a target nucleic acid sequence may comprise bases 141 to 1110 of SEQ ID NO: 22, and a probe typically comprises or is complementary to a nucleic acid sequence having at least 80% sequence identity to a nucleic acid sequence from this target sequence. For example, a target nucleic acid sequence may comprise bases 648 to 709 of SEQ ID NO: 22, and a probe typically comprises or is complementary to a nucleic acid sequence having at least 80% sequence identity to a nucleic acid sequence from this target sequence. In an alternative embodiment, a target nucleic acid sequence may comprise bases 36 to 100 of SEQ ID NO: 22, and a probe typically comprises or is complementary to a nucleic acid sequence having at least 80% sequence identity to a nucleic acid sequence from this target sequence.

In embodiments wherein the one or more biomarker is KLRK1, a target nucleic acid sequence may comprise bases 341 to 1590 of SEQ ID NO: 23, and a probe typically comprises or is complementary to a nucleic acid sequence having at least 80% sequence identity to a nucleic acid sequence from this target sequence.

In embodiments wherein the one or more biomarker is KLRB1, a target nucleic acid sequence may comprise bases 81 to 740 of SEQ ID NO: 24, and a probe typically comprises or is complementary to a nucleic acid sequence having at least 80% sequence identity to a nucleic acid sequence from this target sequence. For example, a target nucleic acid sequence may comprise bases 219 to 291 or 297 to 370 of SEQ ID NO: 24, and a probe typically comprises or is complementary to a nucleic acid sequence having at least 80% sequence identity to a nucleic acid sequence from this target sequence.

In embodiments wherein the one or more biomarker is DAAM2, a target nucleic acid sequence may comprise bases 5131 to 6160 of SEQ ID NO: 25, and a probe typically comprises or is complementary to a nucleic acid sequence having at least 80% sequence identity to a nucleic acid sequence from this target sequence.

In embodiments wherein the one or more biomarker is HLA-DRA, a target nucleic acid sequence may comprise bases 561 to 1210 of SEQ ID NO: 26, and a probe typically comprises or is complementary to a nucleic acid sequence having at least 80% sequence identity to a nucleic acid sequence from this target sequence.

In embodiments wherein the one or more biomarker is BCL11B, a target nucleic acid sequence may comprise bases 3301 to 7670 of SEQ ID NO: 27, and a probe typically comprises or is complementary to a nucleic acid sequence having at least 80% sequence identity to a nucleic acid sequence from this target sequence. In an alternative embodiment, a target nucleic acid sequence may comprise bases 515 to 580 or 532 to 607 of SEQ ID NO: 27, and a probe typically comprises or is complementary to a nucleic acid sequence having at least 80% sequence identity to a nucleic acid sequence from this target sequence.

In embodiments wherein the one or more biomarker is ITM2A, a target nucleic acid sequence may comprise bases 411 to 1250 of SEQ ID NO: 28, and a probe typically comprises or is complementary to a nucleic acid sequence having at least 80% sequence identity to a nucleic acid sequence from this target sequence.

In embodiments wherein the one or more biomarker is SLAMF6, a target nucleic acid sequence may comprise bases 1601 to 2700 of SEQ ID NO: 29, and a probe typically comprises or is complementary to a nucleic acid sequence having at least 80% sequence identity to a nucleic acid sequence from this target sequence.

In embodiments wherein the one or more biomarker is HLA-DPB1, a target nucleic acid sequence may comprise bases 511 to 1090 of SEQ ID NO: 30, or bases 121 to 920 of SEQ ID NO: 31, and a probe typically comprises or is complementary to a nucleic acid sequence having at least 80% sequence identity to a nucleic acid sequence from this target sequence.

In embodiments wherein the one or more biomarker is CD160, a target nucleic acid sequence may comprise bases 871 to 1460 of SEQ ID NO: 32, and a probe typically comprises or is complementary to a nucleic acid sequence having at least 80% sequence identity to a nucleic acid sequence from this target sequence.

In embodiments wherein the one or more biomarker is KLRF1, a target nucleic acid sequence may comprise bases 251 to 1240 of SEQ ID NO: 33, and a probe typically comprises or is complementary to a nucleic acid sequence having at least 80% sequence identity to a nucleic acid sequence from this target sequence.

In embodiments wherein the one or more biomarker is CD2, a target nucleic acid sequence may comprise bases 291 to 1530 of SEQ ID NO: 34, and a probe typically comprises or is complementary to a nucleic acid sequence having at least 80% sequence identity to a nucleic acid sequence from this target sequence.

In embodiments wherein the one or more biomarker is LGALS2, a target nucleic acid sequence may comprise bases 101 to 520 of SEQ ID NO: 35, and a probe typically comprises or is complementary to a nucleic acid sequence having at least 80% sequence identity to a nucleic acid sequence from this target sequence.

In embodiments wherein the one or more biomarker is NPPC, a target nucleic acid sequence may comprise bases 261 to 640 of SEQ ID NO: 36, and a probe typically comprises or is complementary to a nucleic acid sequence having at least 80% sequence identity to a nucleic acid sequence from this target sequence.

In embodiments wherein the one or more biomarker is MYCL, a target nucleic acid sequence may comprise bases 2931 to 3600 of SEQ ID NO: 37, or bases 781 to 1990 of SEQ ID NO: 38, and a probe typically comprises or is complementary to a nucleic acid sequence having at least 80% sequence identity to a nucleic acid sequence from this target sequence. In an alternative embodiment, a target nucleic acid sequence may comprise bases 1022 to 1113 of SEQ ID NO: 37, or bases 661 to 720 of SEQ ID NO: 38, and a probe typically comprises or is complementary to a nucleic acid sequence having at least 80% sequence identity to a nucleic acid sequence from this target sequence

In embodiments wherein the one or more biomarker is MX1, a target nucleic acid sequence may comprise bases 391 to 3400 of SEQ ID NO: 39, and a probe typically comprises or is complementary to a nucleic acid sequence having at least 80% sequence identity to a nucleic acid sequence from this target sequence.

In embodiments wherein the one or more biomarker is CCL5, a target nucleic acid sequence may comprise bases 311 to 1230 of SEQ ID NO: 40, and a probe typically comprises or is complementary to a nucleic acid sequence having at least 80% sequence identity to a nucleic acid sequence from this target sequence.

In embodiments wherein the one or more biomarker is TGFB1, a target nucleic acid sequence may comprise bases 2091 to 2790 of SEQ ID NO: 41, and a probe typically comprises or is complementary to a nucleic acid sequence having at least 80% sequence identity to a nucleic acid sequence from this target sequence. In an alternative embodiment, a target nucleic acid sequence may comprise bases 2228 to 2090 of SEQ ID NO: 41, and a probe typically comprises or is complementary to a nucleic acid sequence having at least 80% sequence identity to a nucleic acid sequence from this target sequence.

In embodiments wherein the one or more biomarker is PLA2G7, a target nucleic acid sequence may comprise bases 1041 to 1810 of SEQ ID NO: 42, and a probe typically comprises or is complementary to a nucleic acid sequence having at least 80% sequence identity to a nucleic acid sequence from this target sequence. In an alternative embodiment, a target nucleic acid sequence may comprise bases 356 to 421 or 608 to 674 of SEQ ID NO: 42, and a probe typically comprises or is complementary to a nucleic acid sequence having at least 80% sequence identity to a nucleic acid sequence from this target sequence.

In embodiments wherein the one or more biomarker is ARHGEF10L, a target nucleic acid sequence may comprise bases 3461 to 4490 of SEQ ID NO: 43, and a probe typically comprises or is complementary to a nucleic acid sequence having at least 80% sequence identity to a nucleic acid sequence from this target sequence. In an alternative embodiment, a target nucleic acid sequence may comprise bases 2275 to 2337 of SEQ ID NO: 43, and a probe typically comprises or is complementary to a nucleic acid sequence having at least 80% sequence identity to a nucleic acid sequence from this target sequence.

In embodiments wherein the one or more biomarker is GPR124, a target nucleic acid sequence may comprise bases 5021 to 5870 of SEQ ID NO: 44, and a probe typically comprises or is complementary to a nucleic acid sequence having at least 80% sequence identity to a nucleic acid sequence from this target sequence.

In embodiments wherein the one or more biomarker is URN, a target nucleic acid sequence may comprise bases 241 to 1920 of SEQ ID NO: 45, and a probe typically comprises or is complementary to a nucleic acid sequence having at least 80% sequence identity to a nucleic acid sequence from this target sequence.

In embodiments wherein the one or more biomarker is NLRP3, a target nucleic acid sequence may comprise bases 1921 to 4160 of SEQ ID NO: 46, and a probe typically comprises or is complementary to a nucleic acid sequence having at least 80% sequence identity to a nucleic acid sequence from this target sequence.

In embodiments wherein the one or more biomarker is RBP4, a target nucleic acid sequence may comprise bases 291 to 940 of SEQ ID NO: 47, and a probe typically comprises or is complementary to a nucleic acid sequence having at least 80% sequence identity to a nucleic acid sequence from this target sequence.

In embodiments wherein the one or more biomarker is MPP3, a target nucleic acid sequence may comprise bases 531 to 2140 of SEQ ID NO: 48, and a probe typically comprises or is complementary to a nucleic acid sequence having at least 80% sequence identity to a nucleic acid sequence from this target sequence.

In embodiments wherein the one or more biomarker is KIF2C, a target nucleic acid sequence may comprise bases 721 to 2630 of SEQ ID NO: 49, and a probe typically comprises or is complementary to a nucleic acid sequence having at least 80% sequence identity to a nucleic acid sequence from this target sequence.

In embodiments wherein the one or more biomarker is MAP1A, a target nucleic acid sequence may comprise bases 9521 to 10275 of SEQ ID NO: 50, and a probe typically comprises or is complementary to a nucleic acid sequence having at least 80% sequence identity to a nucleic acid sequence from this target sequence.

In embodiments wherein the one or more biomarker is SELP, a target nucleic acid sequence may comprise bases 1801 to 3150 of SEQ ID NO: 51, and a probe typically comprises or is complementary to a nucleic acid sequence having at least 80% sequence identity to a nucleic acid sequence from this target sequence.

In embodiments wherein the one or more biomarker is NEXN, a target nucleic acid sequence may comprise bases 361 to 2330 of SEQ ID NO: 52, and a probe typically comprises or is complementary to a nucleic acid sequence having at least 80% sequence identity to a nucleic acid sequence from this target sequence.

In embodiments wherein the one or more biomarker is ITGA2B, a target nucleic acid sequence may comprise bases 2211 to 3300 of SEQ ID NO: 53, and a probe typically comprises or is complementary to a nucleic acid sequence having at least 80% sequence identity to a nucleic acid sequence from this target sequence. For example, a target nucleic acid sequence may comprise bases 2286 to 2345 of SEQ ID NO: 53, and a probe typically comprises or is complementary to a nucleic acid sequence having at least 80% sequence identity to a nucleic acid sequence from this target sequence. In an alternative embodiment, a target nucleic acid sequence may comprise bases 1480 to 1543 of SEQ ID NO: 53, and a probe typically comprises or is complementary to a nucleic acid sequence having at least 80% sequence identity to a nucleic acid sequence from this target sequence.

In embodiments wherein the one or more biomarker is MYL9, a target nucleic acid sequence may comprise bases 221 to 1030 of SEQ ID NO: 54, and a probe typically comprises or is complementary to a nucleic acid sequence having at least 80% sequence identity to a nucleic acid sequence from this target sequence. In an alternative embodiment, a target nucleic acid sequence may comprise bases 38 to 83 or 53 to 120 of SEQ ID NO: 54, and a probe typically comprises or is complementary to a nucleic acid sequence having at least 80% sequence identity to a nucleic acid sequence from this target sequence

In embodiments wherein the one or more biomarker is ITGB3, a target nucleic acid sequence may comprise bases 2611 to 4580 of SEQ ID NO: 55, and a probe typically comprises or is complementary to a nucleic acid sequence having at least 80% sequence identity to a nucleic acid sequence from this target sequence. In an alternative embodiment, a target nucleic acid sequence may comprise bases 1116 to 1182 or 1978 to 2047 of SEQ ID NO: 55, and a probe typically comprises or is complementary to a nucleic acid sequence having at least 80% sequence identity to a nucleic acid sequence from this target sequence.

In embodiments wherein the one or more biomarker is CMTM5, a target nucleic acid sequence may comprise bases 381 to 1020 of SEQ ID NO: 56, and a probe typically comprises or is complementary to a nucleic acid sequence having at least 80% sequence identity to a nucleic acid sequence from this target sequence.

In embodiments wherein the one or more biomarker is LCN2, a target nucleic acid sequence may comprise bases 131 to 710 of SEQ ID NO: 57, and a probe typically comprises or is complementary to a nucleic acid sequence having at least 80% sequence identity to a nucleic acid sequence from this target sequence. For example, a target nucleic acid sequence may comprise bases 532 to 603 or 632 to 689 of SEQ ID NO: 57, and a probe typically comprises or is complementary to a nucleic acid sequence having at least 80% sequence identity to a nucleic acid sequence from this target sequence.

In embodiments wherein the one or more biomarker is NLRC4, a target nucleic acid sequence may comprise bases 441 to 1310 of SEQ ID NO: 58, and a probe typically comprises or is complementary to a nucleic acid sequence having at least 80% sequence identity to a nucleic acid sequence from this target sequence.

In embodiments wherein the one or more biomarker is PPBP, a target nucleic acid sequence may comprise bases 241 to 1200 of SEQ ID NO: 59, and a probe typically comprises or is complementary to a nucleic acid sequence having at least 80% sequence identity to a nucleic acid sequence from this target sequence.

In embodiments wherein the one or more biomarker is TREML1, a target nucleic acid sequence may comprise bases 611 to 1340 of SEQ ID NO: 60, and a probe typically comprises or is complementary to a nucleic acid sequence having at least 80% sequence identity to a nucleic acid sequence from this target sequence. In an alternative embodiment, a target nucleic acid sequence may comprise bases 502 to 569 or 520 to 588 of SEQ ID NO: 60, and a probe typically comprises or is complementary to a nucleic acid sequence having at least 80% sequence identity to a nucleic acid sequence from this target sequence.

In embodiments wherein the one or more biomarker is PF4, a target nucleic acid sequence may comprise bases 261 to 850 of SEQ ID NO: 61, and a probe typically comprises or is complementary to a nucleic acid sequence having at least 80% sequence identity to a nucleic acid sequence from this target sequence.

In embodiments wherein the one or more biomarker is CLEC1B, a target nucleic acid sequence may comprise bases 351 to 970 of SEQ ID NO: 62, and a probe typically comprises or is complementary to a nucleic acid sequence having at least 80% sequence identity to a nucleic acid sequence from this target sequence.

In embodiments wherein the one or more biomarker is LCN15, a target nucleic acid sequence may comprise bases 71 to 762 of SEQ ID NO: 63, and a probe typically comprises or is complementary to a nucleic acid sequence having at least 80% sequence identity to a nucleic acid sequence from this target sequence.

In embodiments wherein the one or more biomarker is CIQC, a target nucleic acid sequence may comprise bases 501 to 1100 of SEQ ID NO: 64, and a probe typically comprises or is complementary to a nucleic acid sequence having at least 80% sequence identity to a nucleic acid sequence from this target sequence. In an alternative embodiment, a target nucleic acid sequence may comprise bases 39 to 302, 39 to 150, 61 to 150 or 61 to 302 of SEQ ID NO: 64, and a probe typically comprises or is complementary to a nucleic acid sequence having at least 80% sequence identity to a nucleic acid sequence from this target sequence.

In embodiments wherein the one or more biomarker is CIQB, a target nucleic acid sequence may comprise bases 321 to 1020 of SEQ ID NO: 65, and a probe typically comprises or is complementary to a nucleic acid sequence having at least 80% sequence identity to a nucleic acid sequence from this target sequence. In an alternative embodiment, a target nucleic acid sequence may comprise bases 91 to 154 or 91 to 157 of SEQ ID NO: 65, and a probe typically comprises or is complementary to a nucleic acid sequence having at least 80% sequence identity to a nucleic acid sequence from this target sequence.

In embodiments wherein the one or more biomarker is PCOLEC2, a target nucleic acid sequence may comprise bases 1091 to 2000 of SEQ ID NO: 66, and a probe typically comprises or is complementary to a nucleic acid sequence having at least 80% sequence identity to a nucleic acid sequence from this target sequence.

In embodiments wherein the one or more biomarker is CIQA, a target nucleic acid sequence may comprise bases 361 to 1098 of SEQ ID NO: 67, and a probe typically comprises or is complementary to a nucleic acid sequence having at least 80% sequence identity to a nucleic acid sequence from this target sequence. In an alternative embodiment, a target nucleic acid sequence may comprise bases 214 to 299 of SEQ ID NO: 67, and a probe typically comprises or is complementary to a nucleic acid sequence having at least 80% sequence identity to a nucleic acid sequence from this target sequence.

In embodiments wherein the one or more biomarker is TMEM37, a target nucleic acid sequence may comprise bases 471 to 1687 of SEQ ID NO: 68, and a probe typically comprises or is complementary to a nucleic acid sequence having at least 80% sequence identity to a nucleic acid sequence from this target sequence. In an alternative embodiment, a target nucleic acid sequence may comprise bases 25 to 115 of SEQ ID NO: 68, and a probe typically comprises or is complementary to a nucleic acid sequence having at least 80% sequence identity to a nucleic acid sequence from this target sequence.

In embodiments wherein the one or more biomarker is TNF, a target nucleic acid sequence may comprise bases 991 to 1670 of SEQ ID NO: 69, and a probe typically comprises or is complementary to a nucleic acid sequence having at least 80% sequence identity to a nucleic acid sequence from this target sequence.

In embodiments wherein the one or more biomarker is SLC39A8, a target nucleic acid sequence may comprise bases 2161 to 3109 of SEQ ID NO: 70, or bases 2921 to 4050 of SEQ ID NO: 71, and a probe typically comprises or is complementary to a nucleic acid sequence having at least 80% sequence identity to a nucleic acid sequence from this target sequence. In an alternative embodiment, a target nucleic acid sequence may comprise bases 1525 to 1603 or 1718 to 1787 of SEQ ID NO: 70, or bases 1360 to 1438 or 1553 to 1622 of SEQ ID NO: 71, and a probe typically comprises or is complementary to a nucleic acid sequence having at least 80% sequence identity to a nucleic acid sequence from this target sequence.

In embodiments wherein the one or more biomarker is MRAS, a target nucleic acid sequence may comprise bases 3581 to 4570 of SEQ ID NO: 72, and a probe typically comprises or is complementary to a nucleic acid sequence having at least 80% sequence identity to a nucleic acid sequence from this target sequence. In an alternative embodiment, a target nucleic acid sequence may comprise bases 1104 to 1167 or 1182 to 1246 of SEQ ID NO: 72, and a probe typically comprises or is complementary to a nucleic acid sequence having at least 80% sequence identity to a nucleic acid sequence from this target sequence.

In embodiments wherein the one or more biomarker is IFIT1, a target nucleic acid sequence may comprise bases 1501 to 3960 of SEQ ID NO: 73, and a probe typically comprises or is complementary to a nucleic acid sequence having at least 80% sequence identity to a nucleic acid sequence from this target sequence.

In embodiments wherein the one or more biomarker is IF144, a target nucleic acid sequence may comprise bases 901 to 1650 of SEQ ID NO: 74, and a probe typically comprises or is complementary to a nucleic acid sequence having at least 80% sequence identity to a nucleic acid sequence from this target sequence.

In embodiments wherein the one or more biomarker is RPGRIP1, a target nucleic acid sequence may comprise bases 2541 to 3770 of SEQ ID NO: 75, and a probe typically comprises or is complementary to a nucleic acid sequence having at least 80% sequence identity to a nucleic acid sequence from this target sequence.

In embodiments wherein the one or more biomarker is DISC1, a target nucleic acid sequence may comprise bases 1201 to 1707 of SEQ ID NO: 76, and a probe typically comprises or is complementary to a nucleic acid sequence having at least 80% sequence identity to a nucleic acid sequence from this target sequence.

In embodiments wherein the one or more biomarker is CXCR1, a target nucleic acid sequence may comprise bases 181 to 2080 of SEQ ID NO: 77, and a probe typically comprises or is complementary to a nucleic acid sequence having at least 80% sequence identity to a nucleic acid sequence from this target sequence. In an alternative embodiment, a target nucleic acid sequence may comprise bases 44 to 113 or 70 to 136 of SEQ ID NO: 77, and a probe typically comprises or is complementary to a nucleic acid sequence having at least 80% sequence identity to a nucleic acid sequence from this target sequence.

In embodiments wherein the one or more biomarker is HCAR2, a target nucleic acid sequence may comprise bases 21 to 1810 of SEQ ID NO: 78, and a probe typically comprises or is complementary to a nucleic acid sequence having at least 80% sequence identity to a nucleic acid sequence from this target sequence. For example, a target nucleic acid sequence may comprise bases 918 to 979 or 1299 to 1356 of SEQ ID NO: 78, and a probe typically comprises or is complementary to a nucleic acid sequence having at least 80% sequence identity to a nucleic acid sequence from this target sequence

In embodiments wherein the one or more biomarker is EPSTI1, a target nucleic acid sequence may comprise bases 621 to 2990 of SEQ ID NO: 79, and a probe typically comprises or is complementary to a nucleic acid sequence having at least 80% sequence identity to a nucleic acid sequence from this target sequence.

In embodiments wherein the one or more biomarker is LILRB4, a target nucleic acid sequence may comprise bases 1081 to 3240 of SEQ ID NO: 80, and a probe typically comprises or is complementary to a nucleic acid sequence having at least 80% sequence identity to a nucleic acid sequence from this target sequence.

In embodiments wherein the one or more biomarker is LILRB5, a target nucleic acid sequence may comprise bases 341 to 2120 of SEQ ID NO: 81, and a probe typically comprises or is complementary to a nucleic acid sequence having at least 80% sequence identity to a nucleic acid sequence from this target sequence. For example, a target nucleic acid sequence may comprise bases 1633 to 1697 or 1653 to 1706 of SEQ ID NO: 81, and a probe typically comprises or is complementary to a nucleic acid sequence having at least 80% sequence identity to a nucleic acid sequence from this target sequence

In embodiments wherein the one or more biomarker is NECAB1, a target nucleic acid sequence may comprise bases 4231 to 5000 of SEQ ID NO: 82, and a probe typically comprises or is complementary to a nucleic acid sequence having at least 80% sequence identity to a nucleic acid sequence from this target sequence. In an alternative embodiment, a target nucleic acid sequence may comprise bases 763 to 845 or 1206 to 1285 of SEQ ID NO: 82, and a probe typically comprises or is complementary to a nucleic acid sequence having at least 80% sequence identity to a nucleic acid sequence from this target sequence.

In embodiments wherein the one or more biomarker is NECAB2, a target nucleic acid sequence may comprise bases 691 to 1490 of SEQ ID NO: 83, and a probe typically comprises or is complementary to a nucleic acid sequence having at least 80% sequence identity to a nucleic acid sequence from this target sequence. In an alternative embodiment, a target nucleic acid sequence may comprise bases 226 to 289 or 579 to 641 of SEQ ID NO: 83, and a probe typically comprises or is complementary to a nucleic acid sequence having at least 80% sequence identity to a nucleic acid sequence from this target sequence.

In embodiments wherein the one or more biomarker is PKHD1, a target nucleic acid sequence may comprise bases 10141 to 16040 of SEQ ID NO: 84, and a probe typically comprises or is complementary to a nucleic acid sequence having at least 80% sequence identity to a nucleic acid sequence from this target sequence. In an alternative embodiment, a target nucleic acid sequence may comprise bases 9037 to 9100 or 10262 to 10335 of SEQ ID NO: 84, and a probe typically comprises or is complementary to a nucleic acid sequence having at least 80% sequence identity to a nucleic acid sequence from this target sequence.

In embodiments wherein the one or more biomarker is PKD1, a target nucleic acid sequence may comprise bases 2201 to 14080 of SEQ ID NO: 85, and a probe typically comprises or is complementary to a nucleic acid sequence having at least 80% sequence identity to a nucleic acid sequence from this target sequence.

It is preferred that the binding conditions for a probe hybridising to its target sequence are such that a high level of specificity is provided—i.e. hybridisation of the probe occurs under “stringent conditions”. In general, stringent conditions are selected to be about 5° C. lower than the thermal melting point (Tm) for the specific sequence at a defined ionic strength and pH. The Tm is the temperature (under defined ionic strength and pH) at which 50% of the target (or complement) sequence hybridises to a perfectly matched probe. In this regard, the Tm of probes of the present invention, at a salt concentration of about 0.02M or less at pH 7, is for example above 60° C., such as about 70° C.

Premixed buffer solutions are commercially available (e.g. EXPRESSHYB Hybridisation Solution from CLONTECH Laboratories, Inc.), and hybridisation can be performed according to the manufacturer's instructions.

Probes of the present invention may be screened to minimise self-complementarity and dimer formation (probe-probe binding).

Any of the probes described herein may comprise a tag and/or label. The tag and/or label may, for example, be located (independently of one another) towards the middle or towards or at the 5′ or 3′ end of the herein described probes, for example at the 5′ end.

Hence, following hybridisation of tagged/labelled probe to target nucleic acid, the tag/label is associated with the target nucleic acid in the one or more biomarker. Alternatively, if an amplification step is employed, the probes may act as primers during the method of the invention and the tag/label may therefore become incorporated into the amplification product as the primer is extended.

Examples of suitable labels include detectable labels such as radiolabels or fluorescent or coloured molecules, enzymatic markers or chromogenic markers—e.g. dyes that produce a visible colour change upon hybridisation of the probe. By way of example, the label may be digoxygenin, fluorescein-isothiocyanate (FITC), R-phycoerythrin, Alexa 532 or Cy3. The probes preferably contain a Fam label (e.g. a 5′ Fam label), and/or a minor groove binder (MGB). The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include “complement/anti-complement pairs”. The term “complement/anti-complement pair” denotes non-identical moieties that form a non-covalently associated, stable pair under appropriate conditions. Examples of suitable tags include biotin and streptavidin (or avidin). By way of example, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary complement/anti-complement pairs include receptor/ligand pairs, antibody/antigen (or hapten or epitope) pairs, and the like. Another example is a nucleic acid sequence tag that binds to a complementary sequence. The latter may itself be pre-labelled, or may be attached to a surface (e.g. a bead) which is separately labelled. An example of the latter embodiment is the well-known LuminexR bead system. Other exemplary pairs of tags and capture molecules include receptor/ligand pairs and antibody/antigen (or hapten or epitope) pairs. Where subsequent dissociation of the complement/anti-complement pair is desirable, the complement/anti-complement pair has a binding affinity of, for example, less than 10⁹ M⁻¹. One exemplary tagged probe is a biotin-labelled probe, which may be detected using horse-radish peroxidase conjugated streptavidin.

The probes of the invention may be labelled with different labels or tags, thereby allowing separate identification of each probe when used in the method of the present invention.

Any conventional method may be employed to attach nucleic acid tags to a probe of the present invention (e.g. to the 5′ end of the defined binding region of the probe). Alternatively, nucleic acid probes of the invention (with pre-attached nucleic acid tags) may be constructed by commercial providers.

If an amplification step is employed, this step may be carried out using methods and platforms known in the art, for example PCR (for example, with the use of “Fast DNA Polymerase”, Life Technologies), such as real-time PCR, block-based PCR, ligase chain reaction, glass capillaries, isothermal amplification methods including loop-mediated isothermal amplification, rolling circle amplification transcription mediated amplification, nucleic acid sequence-based amplification, signal mediated amplification of RNA technology, strand displacement amplification, isothermal multiple displacement amplification, helicase-dependent amplification, single primer isothermal amplification, and circular helicase-dependent amplification. If employed, amplification may be carried using any amplification platform. Preferably, the amplification step may comprise quantitative PCR (real-time PCR).

A general amplification step (e.g. pre-detection) may be employed to increase the amount of the one or more biomarker of the invention present in the sample. PCR amplification primers are typically employed to amplify approximately 100-400 base pair regions of the target/complementary nucleic acid that contain the nucleotide targets of the present invention. In the presence of a suitable polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of target nucleic acid sequences in the one or more biomarker, thereby generating amplification products comprising said target nucleic acid sequences.

An amplification step may be employed in which the probes of the present invention act as primers. In this embodiment, the probes (acting as primers) are extended from their 3′ ends (i.e. in a 5′-to-′3′) direction. Such an amplification step may be employed in conjunction with a general amplification step, such as the one described above.

The detection step may be carried out by any known means. In this regard, the probe or amplification product may be tagged and/or labelled, and the detection method may therefore comprise detecting said tag and/or label.

In one embodiment, the probe(s) may comprise a tag and/or label. Thus, in one embodiment, following hybridisation of tagged/labelled probe to target nucleic acid in the one or more biomarker, the tag/label becomes associated with the target nucleic acid. Thus, in one embodiment, the assay may comprise detecting the tag/label and correlating presence of tag/label with presence of the one or more nucleic acid biomarker of the invention.

In one embodiment, tag and/or label may be incorporated during extension of the probe(s). In doing so, the amplification product(s) become tagged/labelled, and the assay may therefore comprise detecting the tag/label and correlating presence of tag/label with presence of amplification product, and hence the presence of one or more nucleic acid biomarker of the invention.

By way of example, in one embodiment, the amplification product may incorporate a tag/label (e.g. via a tagged/labelled dNTP such as biotin-dNTP) as part of the amplification process, and the assay may further comprise the use of a binding partner complementary to said tag (e.g. streptavidin) that includes a detectable tag/label (e.g. a fluorescent label, such as R-phycoerythrin). In this way, the amplified product incorporates a detectable tag/label (e.g. a fluorescent label, such as R-phycoerythrin).

In one embodiment, the probe(s) and/or the amplification product(s) may include a further tag/label (as the complement component) to allow capture of the amplification product(s).

By way of example, a “complement/anti-complement” pairing may be employed in which an anti-complement capture component binds to said further tag/label (complement component) and thereby permits capture of the probe(s) and/or amplification product(s). Examples of suitable “complement/anti-complement” partners have been described earlier in this specification, such as a complementary pair of nucleic acid sequences, a complementary antibody-antigen pair, etc. The anti-complement capture component may be attached (e.g. coated) on to a substrate or solid support—examples of suitable substrates/supports include membranes and/or beads (e.g. a magnetic or fluorescent bead). Capture methods are well known in the art. For example, LuminexR beads may be employed. Alternatively, the use of magnetic beads may be advantageous because the beads (plus captured, tagged/labelled amplification product) can easily be concentrated and separated from the sample, using conventional techniques known in the art.

Immobilisation provides a physical location for the anti-complement capture component (or probes), and may serve to fix the capture component/probe at a desired location and/or facilitate recovery or separation of probe. The support may be a rigid solid support made from, for example, glass, plastic or silica, such as a bead (for example a fluorescent or magnetic bead). Alternatively, the support may be a membrane, such as nylon or nitrocellulose membrane. 3D matrices are also suitable supports for use with the present invention—e.g. polyacrylamide or PEG gels. Immobilisation to a support/platform may be achieved by a variety of conventional means. By way of example, immobilisation onto a support such as a nylon membrane may be achieved by UV cross-linking. Alternatively, biotin-labelled molecules may be bound to streptavidin-coated substrates (and vice-versa), and molecules prepared with amino linkers may be immobilised on to silanised surfaces. Another means of immobilisation is via a poly-T tail or a poly-C tail, for example at the 3′ or 5′ end. Said immobilisation techniques apply equally to the probe component (and primer pair component, if present) of the present invention.

In one embodiment, the probes of the invention comprise a nucleic acid sequence tag/label (e.g. attached to each probe at the 5′ end of the defined sequence of the probe that binds to target/complement nucleic acid). In more detail, each of the probes is provided with a different nucleic acid sequence tag/label, wherein each of said tags/labels (specifically) binds to a complementary nucleic acid sequence present on the surface of a bead. Each of the different tags/labels binds to its complementary sequence counterpart (and not to any of the complementary sequence counterparts of the other tags), which is located on a uniquely identifiable bead. In this regard, the beads are uniquely identifiable, for example by means of fluorescence at a specific wavelength. Thus, in use, probes of the invention bind to target nucleic acid (if present in the sample). Thereafter, (only) the bound probes may be extended (in the 3′ direction) in the presence of one or more labelled dNTP (e.g. biotin labelled dNTPs, such as biotin-dCTPs).

The extended primers may be contacted with a binding partner counterpart to the labelled dNTPs (e.g. a streptavidin labelled fluorophore, such as streptavidin labelled R-phycoerythrin), which binds to those labelled dNTPs that have become incorporated into the extended primers. Thereafter, the labelled extended primers may be identified by allowing them to bind to their nucleic acid counterparts present on the uniquely identifiable beads. The latter may then be “called” (e.g. to determine the type of bead present by wavelength emission) and the nature of the primer extension (and thus the type of target/complement nucleic acid present) may be determined.

Typically, probes of the invention are oligonucleotides having sequence identity with a region of the one or more biomarker of the invention as disclosed herein. One or more probe may be immobilised on a solid support, and used to interrogate mRNA obtained from a test sample. If the mRNA from the test sample contains the one or more biomarker targeted by the immobilised probe, it will bind to the probe, and may then be detected. The biomarkers of the invention may also be detected using PCR, such as real time PCR.

Any oligonucleotide with the appropriate level of sequence identity with the one or more biomarker of the invention, or with one or more target sequences within said one or more biomarker of the invention may be used as a probe in the methods and uses described herein. Any oligonucleotide with the appropriate level of complementarity with the one or more biomarker of the invention, or with one or more target sequences within said one or more biomarker of the invention may be used as a probe in the methods and uses of the invention described herein. Exemplary sequences of the one or more biomarkers of the invention are given in SEQ ID NOs: 1 to 85 (see Tables 1-4 herein). Exemplary probe nucleic acid sequences for the biomarkers disclosed herein are set out in Table 14 (SEQ ID NOs: 86-421) and are shown as underlined and bold text in the sequences of the Sequence Information section. These probes are best suited to use in microarray detection of the nucleic acid.

Further exemplary probe nucleic acid sequences are set out in Table 15 (SEQ ID NOs: 424, 427, 430, 433, 436, 439, 442, 445, 448, 451, 454, 457, 460, 463, 466, 469, 472, 475, 478, 481, 484, 487, 490, 493, 496, 499, 502, 506, 509, 512, 515, 518, 521, 524, 525, 528, 531, 534, 537, 540, 543, 546, 549, 552, 555, 558, 561, 564, 567, 570, 573, 576, 579, 582, and 585) together with the forward and reverse primers that are preferably used to amplify the target sequence prior to detection. These probes are best suited to use in quantitative PCR.

Any one or more (eg. 2 or more, 3 or more, up to an including all) of the exemplary probe sequences may be used in the methods and uses of the invention to determine the presence and/or amount of the one or more biomarker.

In embodiments wherein the one or more biomarker is ADM, the oligonucleotide probe typically comprises or is complementary to a nucleic acid sequence having at least 80% sequence identity to the nucleic acid sequence of SEQ ID NO: 86, 87, 88 or 89, preferably SEQ ID NO: 86.

In embodiments wherein the one or more biomarker is CD177, the oligonucleotide probe typically comprises or is complementary to a nucleic acid sequence having at least 80% sequence identity to the nucleic acid sequence of SEQ ID NO: 90, 91, 92, or 93, preferably SEQ ID NO: 90.

In embodiments wherein the one or more biomarker is FAM20A, the oligonucleotide probe typically comprises or is complementary to a nucleic acid sequence having at least 80% sequence identity to the nucleic acid sequence of SEQ ID NO: 94, 95, 96 or 97, preferably SEQ ID NO: 94 or 95. Alternatively, the oligonucleotide probe may comprise or is complementary to a nucleic acid sequence having at least 80% sequence identity to the nucleic acid sequence of SEQ ID NO: 424 or 427.

In embodiments wherein the one or more biomarker is IL10, the oligonucleotide probe typically comprises or is complementary to a nucleic acid sequence having at least 80% sequence identity to the nucleic acid sequence of SEQ ID NO: 98, 99, 100 or 101, preferably SEQ ID NO: 98.

In embodiments wherein the one or more biomarker is METT7LB, the oligonucleotide probe typically comprises or is complementary to a nucleic acid sequence having at least 80% sequence identity to the nucleic acid sequence of SEQ ID NO: 102, 103, 104 or 105, preferably SEQ ID NO: 102.

In embodiments wherein the one or more biomarker is MMP9, the oligonucleotide probe typically comprises or is complementary to a nucleic acid sequence having at least 80% sequence identity to the nucleic acid sequence of SEQ ID NO: 106, 107, 108, 109, preferably SEQ ID NO:106.

In embodiments wherein the one or more biomarker is RETN, the oligonucleotide probe typically comprises or is complementary to a nucleic acid sequence having at least 80% sequence identity to the nucleic acid sequence of SEQ ID NO: 110, 111, 112, or 113, preferably SEQ ID NO: 110.

In embodiments wherein the one or more biomarker is TDRD9, the oligonucleotide probe typically comprises or is complementary to a nucleic acid sequence having at least 80% sequence identity to the nucleic acid sequence of SEQ ID NO: 114, 115, 116, or 117, preferably SEQ ID NO: 114.

In embodiments wherein the one or more biomarker is ITGA7, the oligonucleotide probe typically comprises or is complementary to a nucleic acid sequence having at least 80% sequence identity to the nucleic acid sequence of SEQ ID NO: 118, 119, 120, or 121, preferably SEQ ID NO: 118.

In embodiments wherein the one or more biomarker is BMX, the oligonucleotide probe typically comprises or is complementary to a nucleic acid sequence having at least 80% sequence identity to the nucleic acid sequence of SEQ ID NO: 122, 123, 124 or 125, preferably SEQ ID NO: 122.

In embodiments wherein the one or more biomarker is HP, the oligonucleotide probe typically comprises or is complementary to a nucleic acid sequence having at least 80% sequence identity to the nucleic acid sequence of SEQ ID NO: 126, 127, 128 or 129, preferably SEQ ID NO: 126.

In embodiments wherein the one or more biomarker is IGFBP2, the oligonucleotide probe typically comprises or is complementary to a nucleic acid sequence having at least 80% sequence identity to the nucleic acid sequence of SEQ ID NO: 130, 131, 132, or 133, preferably SEQ ID NO: 130.

In embodiments wherein the one or more biomarker is ALPL, the oligonucleotide probe typically comprises or is complementary to a nucleic acid sequence having at least 80% sequence identity to the nucleic acid sequence of SEQ ID NO: 134, 135, 136, or 137, preferably SEQ ID NO: 134.

In embodiments wherein the one or more biomarker is DACH1, the oligonucleotide probe typically comprises or is complementary to a nucleic acid sequence having at least 80% sequence identity to the nucleic acid sequence of SEQ ID NO: 138, 139, 140, or 141, preferably SEQ ID NO: 138 or 139.

In embodiments wherein the one or more biomarker is IL1R1, the oligonucleotide probe typically comprises or is complementary to a nucleic acid sequence having at least 80% sequence identity to the nucleic acid sequence of SEQ ID NO: 142, 143, 144, or 145, preferably SEQ ID NO: 142 or 143.

In embodiments wherein the one or more biomarker is OLAH, the oligonucleotide probe typically comprises or is complementary to a nucleic acid sequence having at least 80% sequence identity to the nucleic acid sequence of SEQ ID NO: 146, 147, 148, or 149, preferably SEQ ID NO: 146. Alternatively, the oligonucleotide probe may comprise or is complementary to a nucleic acid sequence having at least 80% sequence identity to the nucleic acid sequence of SEQ ID NO: 430 or 433.

In embodiments wherein the one or more biomarker is IL1R2, the oligonucleotide probe typically comprises or is complementary to a nucleic acid sequence having at least 80% sequence identity to the nucleic acid sequence of SEQ ID NO: 150, 151, 152, or 153, preferably SEQ ID NO: 150.

In embodiments wherein the one or more biomarker is CYP19A1, the oligonucleotide probe typically comprises or is complementary to a nucleic acid sequence having at least 80% sequence identity to the nucleic acid sequence of SEQ ID NO: 154, 155, 156 or 157, preferably SEQ ID NO: 154 or 155.

In embodiments wherein the one or more biomarker is MMP8, the oligonucleotide probe typically comprises or is complementary to a nucleic acid sequence having at least 80% sequence identity to the nucleic acid sequence of SEQ ID NO: 158, 159, 160, or 161, preferably SEQ ID NO: 158.

In embodiments wherein the one or more biomarker is TGFA, the oligonucleotide probe typically comprises or is complementary to a nucleic acid sequence having at least 80% sequence identity to the nucleic acid sequence of SEQ ID NO: 162, 163, 164, 165, preferably SEQ ID NO: 162.

In embodiments wherein the one or more biomarker is VSTM1, the oligonucleotide probe typically comprises or is complementary to a nucleic acid sequence having at least 80% sequence identity to the nucleic acid sequence of SEQ ID NO: 166, 167, 168, or 169, preferably SEQ ID NO:166.

In embodiments wherein the one or more biomarker is FCER1A, the oligonucleotide probe typically comprises or is complementary to a nucleic acid sequence having at least 80% sequence identity to the nucleic acid sequence of SEQ ID NO: 170, 171, 172, or 173, preferably SEQ ID NO:170. Alternatively, the oligonucleotide probe may comprise or is complementary to a nucleic acid sequence having at least 80% sequence identity to the nucleic acid sequence of SEQ ID NO: 528 or 531.

In embodiments wherein the one or more biomarker is KLRK1, the oligonucleotide probe typically comprises or is complementary to a nucleic acid sequence having at least 80% sequence identity to the nucleic acid sequence of SEQ ID NO:174, 175, 176, or 177, preferably SEQ ID NO: 174.

In embodiments wherein the one or more biomarker is KLRB1, the oligonucleotide probe typically comprises or is complementary to a nucleic acid sequence having at least 80% sequence identity to the nucleic acid sequence of SEQ ID NO: 178, 179, 180, or 181, preferably SEQ ID NO: 178. Alternatively, the oligonucleotide probe may comprise or is complementary to a nucleic acid sequence having at least 80% sequence identity to the nucleic acid sequence of SEQ ID NO: 546 or 549.

In embodiments wherein the one or more biomarker is DAAM2, the oligonucleotide probe typically comprises or is complementary to a nucleic acid sequence having at least 80% sequence identity to the nucleic acid sequence of SEQ ID NO: 182, 183, 184, or 185, preferably SEQ ID NO: 182 or 183.

In embodiments wherein the one or more biomarker is HLA-DRA, the oligonucleotide probe typically comprises or is complementary to a nucleic acid sequence having at least 80% sequence identity to the nucleic acid sequence of SEQ ID NO: 186, 187, 188, or 189, preferably SEQ ID NO:186.

In embodiments wherein the one or more biomarker is BCL11B, the oligonucleotide probe typically comprises or is complementary to a nucleic acid sequence having at least 80% sequence identity to the nucleic acid sequence of SEQ ID NO: 190, 191, 192, or 193, preferably SEQ ID NO: 190 or 191. Alternatively, the oligonucleotide probe may comprise or is complementary to a nucleic acid sequence having at least 80% sequence identity to the nucleic acid sequence of SEQ ID NO: 534 or 537.

In embodiments wherein the one or more biomarker is ITM2A, the oligonucleotide probe typically comprises or is complementary to a nucleic acid sequence having at least 80% sequence identity to the nucleic acid sequence of SEQ ID NO: 194, 195, 196, or 197, preferably SEQ ID NO: 194.

In embodiments wherein the one or more biomarker is SLAMF6, the oligonucleotide probe typically comprises or is complementary to a nucleic acid sequence having at least 80% sequence identity to the nucleic acid sequence of SEQ ID NO: 198, 199, 200, or 201, preferably SEQ ID NO: 198.

In embodiments wherein the one or more biomarker is HLA-DPB1, the oligonucleotide probe typically comprises or is complementary to a nucleic acid sequence having at least 80% sequence identity to the nucleic acid sequence of SEQ ID NO: 202, 203, 204, or 205, preferably SEQ ID NO:202 or 203.

In embodiments wherein the one or more biomarker is CD160, the oligonucleotide probe typically comprises or is complementary to a nucleic acid sequence having at least 80% sequence identity to the nucleic acid sequence of SEQ ID NO: 206, 207, 208, or 209, preferably SEQ ID NO: 206.

In embodiments wherein the one or more biomarker is KLFF1, the oligonucleotide probe typically comprises or is complementary to a nucleic acid sequence having at least 80% sequence identity to the nucleic acid sequence of SEQ ID NO: 210, 211, 212, or 213, preferably SEQ ID NO: 210.

In embodiments wherein the one or more biomarker is CD2, the oligonucleotide probe typically comprises or is complementary to a nucleic acid sequence having at least 80% sequence identity to the nucleic acid sequence of SEQ ID NO:214, 215, 216 or 217, preferably SEQ ID NO: 214.

In embodiments wherein the one or more biomarker is LGALS2, the oligonucleotide probe typically comprises or is complementary to a nucleic acid sequence having at least 80% sequence identity to the nucleic acid sequence of SEQ ID NO: 218, 219, 220, or 221, preferably SEQ ID NO: 218.

In embodiments wherein the one or more biomarker is NPPC, the oligonucleotide probe typically comprises or is complementary to a nucleic acid sequence having at least 80% sequence identity to the nucleic acid sequence of SEQ ID NO: 222, 223, 224, or 225, preferably SEQ ID NO: 222.

In embodiments wherein the one or more biomarker is MYCL, the oligonucleotide probe typically comprises or is complementary to a nucleic acid sequence having at least 80% sequence identity to the nucleic acid sequence of SEQ ID NO: 226, 227, 228, 229, 230, 231, 232, or 233. In embodiments wherein the one or more biomarker is transcript variant 3 of MYCL, the oligonucleotide probe typically comprises or is complementary to a nucleic acid sequence having at least 80% sequence identity to the nucleic acid sequence of SEQ ID NO: 226, 227, 228, or 229, preferably SEQ ID NO: 226. In embodiments wherein the one or more biomarker is transcript variant 1 of MYCL, the oligonucleotide probe typically comprises or is complementary to a nucleic acid sequence having at least 80% sequence identity to the nucleic acid sequence of SEQ ID NO: 230, 231, 232, or 233, preferably SEQ ID NO: 230. Alternatively, the oligonucleotide probe may comprise or is complementary to a nucleic acid sequence having at least 80% sequence identity to the nucleic acid sequence of SEQ ID NO: 481 or 484.

In embodiments wherein the one or more biomarker is MX1, the oligonucleotide probe typically comprises or is complementary to a nucleic acid sequence having at least 80% sequence identity to the nucleic acid sequence of SEQ ID NO: 234, 235, 236, or 237, preferably SEQ ID NO: 234.

In embodiments wherein the one or more biomarker is CCL5, the oligonucleotide probe typically comprises or is complementary to a nucleic acid sequence having at least 80% sequence identity to the nucleic acid sequence of SEQ ID NO: 238, 239, 240, or 241, preferably SEQ ID NO: 238.

In embodiments wherein the one or more biomarker is TGFB1, the oligonucleotide probe typically comprises or is complementary to a nucleic acid sequence having at least 80% sequence identity to the nucleic acid sequence of SEQ ID NO: 242, 243, 244, or 245, preferably SEQ ID NO: 242. Alternatively, the oligonucleotide probe may comprise or is complementary to a nucleic acid sequence having at least 80% sequence identity to the nucleic acid sequence of SEQ ID NO: 475 or 478.

In embodiments wherein the one or more biomarker is PLA2G7, the oligonucleotide probe typically comprises or is complementary to a nucleic acid sequence having at least 80% sequence identity to the nucleic acid sequence of SEQ ID NO: 246, 247, 248, or 249, preferably SEQ ID NO: 246. Alternatively, the oligonucleotide probe may comprise or is complementary to a nucleic acid sequence having at least 80% sequence identity to the nucleic acid sequence of SEQ ID NO: 466 or 469.

In embodiments wherein the one or more biomarker is ARHGEF10L, the oligonucleotide probe typically comprises or is complementary to a nucleic acid sequence having at least 80% sequence identity to the nucleic acid sequence of SEQ ID NO: 250, 251, 252, or 253, preferably SEQ ID NO: 250 or 251. Alternatively, the oligonucleotide probe may comprise or is complementary to a nucleic acid sequence having at least 80% sequence identity to the nucleic acid sequence of SEQ ID NO: 472.

In embodiments wherein the one or more biomarker is GPR124, the oligonucleotide probe typically comprises or is complementary to a nucleic acid sequence having at least 80% sequence identity to the nucleic acid sequence of SEQ ID NO: 254, 255, 256, or 257, preferably SEQ ID NO: 254.

In embodiments wherein the one or more biomarker is URN, the oligonucleotide probe typically comprises or is complementary to a nucleic acid sequence having at least 80% sequence identity to the nucleic acid sequence of SEQ ID NO: 258, 259, 260, or 261, preferably SEQ ID NO: 258 or 259.

In embodiments wherein the one or more biomarker is NLRP3, the oligonucleotide probe typically comprises or is complementary to a nucleic acid sequence having at least 80% sequence identity to the nucleic acid sequence of SEQ ID NO: 262, 263, 264, or 265, preferably SEQ ID NO: 262.

In embodiments wherein the one or more biomarker is RBP4, the oligonucleotide probe typically comprises or is complementary to a nucleic acid sequence having at least 80% sequence identity to the nucleic acid sequence of SEQ ID NO: 266, 267, 268, or 269, preferably SEQ ID NO: 266.

In embodiments wherein the one or more biomarker is MPP3, the oligonucleotide probe typically comprises or is complementary to a nucleic acid sequence having at least 80% sequence identity to the nucleic acid sequence of SEQ ID NO: 270, 271, 272, or 273, preferably SEQ ID NO: 270.

In embodiments wherein the one or more biomarker is KIF2C, the oligonucleotide probe typically comprises or is complementary to a nucleic acid sequence having at least 80% sequence identity to the nucleic acid sequence of SEQ ID NO: 274, 275, 276, or 277, preferably SEQ ID NO:274.

In embodiments wherein the one or more biomarker is MAP1A, the oligonucleotide probe typically comprises or is complementary to a nucleic acid sequence having at least 80% sequence identity to the nucleic acid sequence of SEQ ID NO: 278, 279, 280, or 281, preferably SEQ ID NO: 278.

In embodiments wherein the one or more biomarker is SELP, the oligonucleotide probe typically comprises or is complementary to a nucleic acid sequence having at least 80% sequence identity to the nucleic acid sequence of SEQ ID NO: 282, 283, 284, or 285, preferably SEQ ID NO: 282.

In embodiments wherein the one or more biomarker is NEXN, the oligonucleotide probe typically comprises or is complementary to a nucleic acid sequence having at least 80% sequence identity to the nucleic acid sequence of SEQ ID NO: 286, 287, 288, or 289, preferably SEQ ID NO:286 or 287.

In embodiments wherein the one or more biomarker is ITGA2B, the oligonucleotide probe typically comprises or is complementary to a nucleic acid sequence having at least 80% sequence identity to the nucleic acid sequence of SEQ ID NO: 290, 291, 292, or 293, preferably SEQ ID NO: 290. Alternatively, the oligonucleotide probe may comprise or is complementary to a nucleic acid sequence having at least 80% sequence identity to the nucleic acid sequence of SEQ ID NO: 460 or 463.

In embodiments wherein the one or more biomarker is MYL9, the oligonucleotide probe typically comprises or is complementary to a nucleic acid sequence having at least 80% sequence identity to the nucleic acid sequence of SEQ ID NO: 294, 295, 296, or 297, preferably SEQ ID NO: 294. Alternatively, the oligonucleotide probe may comprise or is complementary to a nucleic acid sequence having at least 80% sequence identity to the nucleic acid sequence of SEQ ID NO: 448 or 451.

In embodiments wherein the one or more biomarker is ITGB3, the oligonucleotide probe typically comprises or is complementary to a nucleic acid sequence having at least 80% sequence identity to the nucleic acid sequence of SEQ ID NO: 298, 299, 300, or 301, preferably SEQ ID NO: 298.

In embodiments wherein the one or more biomarker is CMTM5, the oligonucleotide probe typically comprises or is complementary to a nucleic acid sequence having at least 80% sequence identity to the nucleic acid sequence of SEQ ID NO: 302, 303, 304 or 305, preferably SEQ ID NO: 302.

In embodiments wherein the one or more biomarker is LCN2, the oligonucleotide probe typically comprises or is complementary to a nucleic acid sequence having at least 80% sequence identity to the nucleic acid sequence of SEQ ID NO: 306, 307, 308, or 309, preferably SEQ ID NO: 306. Alternatively, the oligonucleotide probe may comprise or is complementary to a nucleic acid sequence having at least 80% sequence identity to the nucleic acid sequence of SEQ ID NO: 436 or 439.

In embodiments wherein the one or more biomarker is NLRC4, the oligonucleotide probe typically comprises or is complementary to a nucleic acid sequence having at least 80% sequence identity to the nucleic acid sequence of SEQ ID NO: 310, 311, 312, or 313, preferably SEQ ID NO: 310.

In embodiments wherein the one or more biomarker is PPBP, the oligonucleotide probe typically comprises or is complementary to a nucleic acid sequence having at least 80% sequence identity to the nucleic acid sequence of SEQ ID NO: 314, 315, 316, or 317, preferably SEQ ID NO: 314.

In embodiments wherein the one or more biomarker is TREML1, the oligonucleotide probe typically comprises or is complementary to a nucleic acid sequence having at least 80% sequence identity to the nucleic acid sequence of SEQ ID NO: 318, 319, 320, 321, preferably SEQ ID NO: 318.

In embodiments wherein the one or more biomarker is PF4, the oligonucleotide probe typically comprises or is complementary to a nucleic acid sequence having at least 80% sequence identity to the nucleic acid sequence of SEQ ID NO: 322, 323, 324, or 325, preferably SEQ ID NO: 322.

In embodiments wherein the one or more biomarker is CLEC1B, the oligonucleotide probe typically comprises or is complementary to a nucleic acid sequence having at least 80% sequence identity to the nucleic acid sequence of SEQ ID NO: 326, 327, 328, or 329, preferably SEQ ID NO: 326 or 327.

In embodiments wherein the one or more biomarker is LCN15, the oligonucleotide probe typically comprises or is complementary to a nucleic acid sequence having at least 80% sequence identity to the nucleic acid sequence of SEQ ID NO: 330, 331, 332, or 333, preferably SEQ ID NO: 330.

In embodiments wherein the one or more biomarker is CIQC, the oligonucleotide probe typically comprises or is complementary to a nucleic acid sequence having at least 80% sequence identity to the nucleic acid sequence of SEQ ID NO: 334, 335, 336, or 337, preferably SEQ ID NO: 334. Alternatively, the oligonucleotide probe may comprise or is complementary to a nucleic acid sequence having at least 80% sequence identity to the nucleic acid sequence of SEQ ID NO: 506 or 509.

In embodiments wherein the one or more biomarker is CIQB, the oligonucleotide probe typically comprises or is complementary to a nucleic acid sequence having at least 80% sequence identity to the nucleic acid sequence of SEQ ID NO: 338, 339, 340, or 341, preferably SEQ ID NO: 338. Alternatively, the oligonucleotide probe may comprise or is complementary to a nucleic acid sequence having at least 80% sequence identity to the nucleic acid sequence of SEQ ID NO: 502.

In embodiments wherein the one or more biomarker is PCOLCE2, the oligonucleotide probe typically comprises or is complementary to a nucleic acid sequence having at least 80% sequence identity to the nucleic acid sequence of SEQ ID NO: 342, 343, 344, or 345, preferably SEQ ID NO: 342.

In embodiments wherein the one or more biomarker is CIQA, the oligonucleotide probe typically comprises or is complementary to a nucleic acid sequence having at least 80% sequence identity to the nucleic acid sequence of SEQ ID NO: 346, 347, 348, or 349, preferably SEQ ID NO: 346. Alternatively, the oligonucleotide probe may comprise or is complementary to a nucleic acid sequence having at least 80% sequence identity to the nucleic acid sequence of SEQ ID NO: 499.

In embodiments wherein the one or more biomarker is TMEM37, the oligonucleotide probe typically comprises or is complementary to a nucleic acid sequence having at least 80% sequence identity to the nucleic acid sequence of SEQ ID NO: 350, 351, 352, or 353, preferably SEQ ID NO: 350. Alternatively, the oligonucleotide probe may comprise or is complementary to a nucleic acid sequence having at least 80% sequence identity to the nucleic acid sequence of SEQ ID NO: 524 or 525.

In embodiments wherein the one or more biomarker is TNF, the oligonucleotide probe typically comprises or is complementary to a nucleic acid sequence having at least 80% sequence identity to the nucleic acid sequence of SEQ ID NO: 354, 355, 356, or 357, preferably SEQ ID NO: 354.

In embodiments wherein the one or more biomarker is SLC39A8, the oligonucleotide probe typically comprises or is complementary to a nucleic acid sequence having at least 80% sequence identity to the nucleic acid sequence of SEQ ID NO: 358, 359, 360, 361, 362, 363, 364, or 365, preferably SEQ ID NO: 358 or 362. In embodiments wherein the one or more biomarker is transcript variant 1 of SLC39A8, the oligonucleotide probe typically comprises or is complementary to a nucleic acid sequence having at least 80% sequence identity to the nucleic acid sequence of SEQ ID NO: 358, 359, 360, or 361, preferably SEQ ID NO: 358. In embodiments wherein the one or more biomarker is transcript variant 3 of SLC39A8, the oligonucleotide probe typically comprises or is complementary to a nucleic acid sequence having at least 80% sequence identity to the nucleic acid sequence of SEQ ID NO: 362, 363, 364, or 365, preferably SEQ ID NO: 362. Alternatively, the oligonucleotide probe may comprise or is complementary to a nucleic acid sequence having at least 80% sequence identity to the nucleic acid sequence of SEQ ID NO: 518 or 521.

In embodiments wherein the one or more biomarker is MRAS, the oligonucleotide probe typically comprises or is complementary to a nucleic acid sequence having at least 80% sequence identity to the nucleic acid sequence of SEQ ID NO: 366, 367, 368, or 369, preferably SEQ ID NO: 366. Alternatively, the oligonucleotide probe may comprise or is complementary to a nucleic acid sequence having at least 80% sequence identity to the nucleic acid sequence of SEQ ID NO: 512 or 515.

In embodiments wherein the one or more biomarker is IFIT1, the oligonucleotide probe typically comprises or is complementary to a nucleic acid sequence having at least 80% sequence identity to the nucleic acid sequence of SEQ ID NO: 370, 371, 372, or 373, preferably SEQ ID NO: 370.

In embodiments wherein the one or more biomarker is IF144, the oligonucleotide probe typically comprises or is complementary to a nucleic acid sequence having at least 80% sequence identity to the nucleic acid sequence of SEQ ID NO: 374, 375, 376, or 377, preferably SEQ ID NO: 374.

In embodiments wherein the one or more biomarker is RPGRIP1, the oligonucleotide probe typically comprises or is complementary to a nucleic acid sequence having at least 80% sequence identity to the nucleic acid sequence of SEQ ID NO: 378, 379, 380, or 381, preferably SEQ ID NO: 378.

In embodiments wherein the one or more biomarker is DISC1, the oligonucleotide probe typically comprises or is complementary to a nucleic acid sequence having at least 80% sequence identity to the nucleic acid sequence of SEQ ID NO: 382, 383, 384, or 385, preferably SEQ ID NO: 382.

In embodiments wherein the one or more biomarker is CXCR1, the oligonucleotide probe typically comprises or is complementary to a nucleic acid sequence having at least 80% sequence identity to the nucleic acid sequence of SEQ ID NO: 386, 387, 388, or 389, preferably SEQ ID NO: 386. Alternatively, the oligonucleotide probe may comprise or is complementary to a nucleic acid sequence having at least 80% sequence identity to the nucleic acid sequence of SEQ ID NO: 487 or 490.

In embodiments wherein the one or more biomarker is HCAR2, the oligonucleotide probe typically comprises or is complementary to a nucleic acid sequence having at least 80% sequence identity to the nucleic acid sequence of SEQ ID NO: 390, 391, 392, or 393, preferably SEQ ID NO: 390. Alternatively, the oligonucleotide probe may comprise or is complementary to a nucleic acid sequence having at least 80% sequence identity to the nucleic acid sequence of SEQ ID NO: 493 or 496.

In embodiments wherein the one or more biomarker is EPST1, the oligonucleotide probe typically comprises or is complementary to a nucleic acid sequence having at least 80% sequence identity to the nucleic acid sequence of SEQ ID NO: 394, 395, 396, or 397, preferably SEQ ID NO: 394.

In embodiments wherein the one or more biomarker is LILRB4, the oligonucleotide probe typically comprises or is complementary to a nucleic acid sequence having at least 80% sequence identity to the nucleic acid sequence of SEQ ID NO: 398, 399, 400, or 401, preferably SEQ ID NO: 398 and 399.

In embodiments wherein the one or more biomarker is LILRB5, the oligonucleotide probe typically comprises or is complementary to a nucleic acid sequence having at least 80% sequence identity to the nucleic acid sequence of SEQ ID NO: 402, 403, 404, or 405, preferably SEQ ID NO: 402. Alternatively, the oligonucleotide probe may comprise or is complementary to a nucleic acid sequence having at least 80% sequence identity to the nucleic acid sequence of SEQ ID NO: 552 or 555.

In embodiments wherein the one or more biomarker is NECAB1, the oligonucleotide probe typically comprises or is complementary to a nucleic acid sequence having at least 80% sequence identity to the nucleic acid sequence of SEQ ID NO: 406, 407, 408, or 409, preferably SEQ ID NO: 406. Alternatively, the oligonucleotide probe may comprise or is complementary to a nucleic acid sequence having at least 80% sequence identity to the nucleic acid sequence of SEQ ID NO: 558 or 561.

In embodiments wherein the one or more biomarker is NECAB2, the oligonucleotide probe typically comprises or is complementary to a nucleic acid sequence having at least 80% sequence identity to the nucleic acid sequence of SEQ ID NO: 410, 411, 412, or 413, preferably SEQ ID NO: 410. Alternatively, the oligonucleotide probe may comprise or is complementary to a nucleic acid sequence having at least 80% sequence identity to the nucleic acid sequence of SEQ ID NO: 564 or 567.

In embodiments wherein the one or more biomarker is PKHD1, the oligonucleotide probe typically comprises or is complementary to a nucleic acid sequence having at least 80% sequence identity to the nucleic acid sequence of SEQ ID NO: 414, 415, 416 or 417, preferably SEQ ID NO: 414 or 415. Alternatively, the oligonucleotide probe may comprise or is complementary to a nucleic acid sequence having at least 80% sequence identity to the nucleic acid sequence of SEQ ID NO: 540 or 543.

In embodiments wherein the one or more biomarker is PKD1, the oligonucleotide probe typically comprises or is complementary to a nucleic acid sequence having at least 80% sequence identity to the nucleic acid sequence of SEQ ID NO: 418, 419, 420, or 421, preferably SEQ ID NO: 418.

In all of the methods and uses described herein, the presence and/or amount of the one or more biomarker is determined using an oligonucleotide probe specific for the one or more biomarker. The oligonucleotide probe used in the methods and uses of the invention may an oligonucleotide probe of the invention as described herein.

As described above, a general amplification step (e.g. pre-detection) may be employed to increase the amount of the one or more biomarker of the invention present in the sample. As well as using the oligonucleotide probes of the invention as primers, separate forward and reverse oligonucleotide primers may be used to amplify a target nucleic acid sequence. The amplified nucleic acid may be detected using an oligonucleotide probe of the invention. For example, such primers and probes may be used when the one or more biomarker is detected and/or quantified by quantitative PCR.

The present invention therefore provides a forward oligonucleotide primer and/or a reverse oligonucleotide primer for amplification of a target nucleic acid sequence in the one or more biomarker.

In one embodiment, one or more forward oligonucleotide primer and one or more reverse oligonucleotide primer may be used to amplify the one or more nucleic acid biomarker of the invention prior to detection.

In general, a reverse primer is designed to hybridise to a target nucleic acid sequence within the coding (sense) strand of a target nucleic acid, and a forward primer is designed to hybridise to a target nucleic acid sequence within the complementary (ie. anti-sense) strand of the target nucleic acid.

The term “complement of a nucleic acid sequence” refers to a nucleic acid sequence having a complementary nucleotide sequence and reverse orientation as compared to a reference nucleotide sequence.

The forward primer hybridises to a target nucleic acid sequence (a ‘forward primer target sequence’) located within the sequence of the nucleic acid biomarker. In one embodiment, the forward primer target sequence has a length in the range of 10-40 consecutive nucleotides, such at least 11, 12, 13, 14, 15, 16, 17, 18, 19, 20, 21 or 22 consecutive nucleotides, and/or up to 38, 35, 32, 30, 29, 28, 27, 26, 25, 24, 23, 22, 21 or 20 consecutive nucleotides.

The reverse primer hybridises to a target nucleic acid sequence (a ‘reverse primer target sequence’) located within the sequence of the nucleic acid biomarker. In one embodiment, the reverse primer target sequence has a length in the range of 10-40 consecutive nucleotides, such as at least 11, 12, 13, 14, 15, 16, 17, 18, 19, 20, 21 or 22 consecutive nucleotides, and/or up to 38, 35, 32, 30, 29, 28, 27, 26, 25, 24, 23, 22, 21 or 20 consecutive nucleotides.

The present invention also provides oligonucleotide primers and probes for amplifying control (or reference) genes. In one embodiment, the control gene is selected from the group consisting of: ALAS1 (NM_000688 SEQ ID NO: 586, or NM_199166, SEQ ID NO: 587), GTF2D1 (NM_003194, SEQ ID NO: 588, and HMBS (NM_000190.3, SEQ ID NO: 589).

In one embodiment, the forward primer hybridises to a target nucleic acid sequence that comprises (or consists of) the complement of a nucleotide sequence selected from SEQ ID NOs: 1-85, or a nucleotide sequence that is at least 80% identical thereto (e.g. at least 82, 84. 86, 88, 90, 91, 92, 93, 94, 95, 96, 97, 98 or 99% identical thereto).

In one embodiment, the reverse primer hybridises to a target nucleic acid sequence that comprises (or consists of) a nucleotide sequence that is at least 80% identical to (eg. at least 82, 84, 86, 88, 90, 91, 92, 93, 94, 95, 96, 97, 98, 99 or 100% identical to) a nucleotide sequence selected from SEQ ID NOs: 1-85.

Exemplary primer and probe sequences are shown in Table 15.

In one embodiment, the forward primer hybridises to a target nucleic acid sequence that comprises (or consists of) the complement of a nucleotide sequence selected from SEQ ID NOs: 422, 425, 428, 431, 434, 437, 440, 443, 446, 449, 452, 455, 458, 461, 464, 467, 470, 473, 476, 479, 482, 485, 488, 491, 494, 497, 500, 504, 507, 510, 513, 516, 519, 522, 526, 529, 532, 535, 538, 541, 544, 547, 550, 553, 556, 559, 562, 565, 568, 571, 574, 577, 580, and 583 (as shown in Table 15), or a nucleotide sequence that is at least 80% identical thereto (e.g. at least 82, 84. 86, 88, 90, 91, 92, 93, 94, 95, 96, 97, 98 or 99% identical thereto).

In one embodiment, the reverse primer hybridises to a target nucleic acid sequence that comprises (or consists of) the complement of a nucleotide sequence that is at least 80% identical to (eg. at least 82, 84. 86, 88, 90, 91, 92, 93, 94, 95, 96, 97, 98, 99 or 100% identical to) a nucleotide sequence selected from SEQ ID NOs: 423, 426, 429, 432, 435, 438, 441, 444, 447, 450, 453, 456, 447, 450, 453, 456, 459, 462, 465, 468, 471, 474, 477, 480, 483, 486, 489, 492, 495, 498, 501, 503, 505, 508, 511, 514, 517, 520, 523, 527, 530, 533, 536, 539, 542, 545, 548, 551, 554, 557, 560, 563, 566, 569, 572, 575, 578, 581, and 584 (as shown in Table 15).

In one embodiment, the forward primer comprises (or consists of) a nucleotide sequence having at least 80% identity to (preferably at least 82, 84, 86, 88, 90 91, 92, 93, 94, 95, 96, 97, 98, 99 or 100% identity to) a nucleotide sequence selected from SEQ ID NOs: 422, 425, 428, 431, 434, 437, 440, 443, 446, 449, 452, 455, 458, 461, 464, 467, 470, 473, 476, 479, 482, 485, 488, 491, 494, 497, 500, 504, 507, 510, 513, 516, 519, 522, 526, 529, 532, 535, 538, 541, 544, 547, 550, 553, 556, 559, 562, 565, 568, 571, 574, 577, 580, and 583 (as shown in Table 15). Conservative substitutions are preferred.

In one embodiment, the reverse primer comprises (or consists of) a nucleotide sequence having at least 80% identity to (preferably at least 82, 84, 86, 88, 90 91, 92, 93, 94, 95, 96, 97, 98, 99 or 100% identity to) a nucleotide sequence selected from SEQ ID NOs: 423, 426, 429, 432, 435, 438, 441, 444, 447, 450, 453, 456, 447, 450, 453, 456, 459, 462, 465, 468, 471, 474, 477, 480, 483, 486, 489, 492, 495, 498, 501, 503, 505, 508, 511, 514, 517, 520, 523, 527, 530, 533, 536, 539, 542, 545, 548, 551, 554, 557, 560, 563, 566, 569, 572, 575, 578, 581, and 584 (as shown in Table 15). Conservative substitutions are preferred.

In embodiments wherein the one or more biomarker is FAM20A, the forward primer hybridises to a target nucleic acid sequence that comprises (or consists of) the complement of the nucleotide sequence of SEQ ID NOs: 422 or 425, or a nucleotide sequence that is at least 80% identical thereto. For example, the forward primer comprises (or consists of) a nucleotide sequence having at least 80% identity to the nucleic acid sequence of SEQ ID NO: 422 or 425. The reverse primer hybridises to a target nucleic acid sequence that comprises (or consists of) the complement of a nucleotide sequence that is at least 80% identical to the nucleic acid sequence of SEQ ID NOs: 423 or 426. For example, the reverse primer comprises (or consists of) a nucleotide sequence having at least 80% identity to the nucleic acid sequence of SEQ ID NOs: 423 or 426. The amplified target nucleic acid may be detected using an oligonucleotide probe as described herein (e.g. by reference to SEQ ID NOs: 424 or 427.

In embodiments wherein the one or more biomarker is OLAH, the forward primer hybridises to a target nucleic acid sequence that comprises (or consists of) the complement of the nucleotide sequence of SEQ ID NOs: 428 or 431, or a nucleotide sequence that is at least 80% identical thereto. For example, the forward primer comprises (or consists of) a nucleotide sequence having at least 80% identity to the nucleic acid sequence of SEQ ID NO: 428 or 431. The reverse primer hybridises to a target nucleic acid sequence that comprises (or consists of) the complement of a nucleotide sequence that is at least 80% identical to the nucleic acid sequence of SEQ ID NOs: 429 or 432. For example, the reverse primer comprises (or consists of) a nucleotide sequence having at least 80% identity to the nucleic acid sequence of SEQ ID NOs: 429 or 432. The amplified target nucleic acid may be detected using an oligonucleotide probe as described herein (e.g. by reference to SEQ ID NOs: 430 or 433).

In embodiments wherein the one or more biomarker is LCN2, the forward primer hybridises to a target nucleic acid sequence that comprises (or consists of) the complement of the nucleotide sequence of SEQ ID NOs: 434 or 437, or a nucleotide sequence that is at least 80% identical thereto. For example, the forward primer comprises (or consists of) a nucleotide sequence having at least 80% identity to the nucleic acid sequence of SEQ ID NO: 434 or 437. The reverse primer hybridises to a target nucleic acid sequence that comprises (or consists of) the complement of a nucleotide sequence that is at least 80% identical to the nucleic acid sequence of SEQ ID NOs: 435 or 438. For example, the reverse primer comprises (or consists of) a nucleotide sequence having at least 80% identity to the nucleic acid sequence of SEQ ID NOs: 435 or 438. The amplified target nucleic acid may be detected using an oligonucleotide probe as described herein (e.g. by reference to SEQ ID NOs: 436 or 439).

In embodiments wherein the one or more biomarker is ITGB3, the forward primer hybridises to a target nucleic acid sequence that comprises (or consists of) the complement of the nucleotide sequence of SEQ ID NOs: 440 or 443, or a nucleotide sequence that is at least 80% identical thereto. For example, the forward primer comprises (or consists of) a nucleotide sequence having at least 80% identity to the nucleic acid sequence of SEQ ID NO: 440 or 443. The reverse primer hybridises to a target nucleic acid sequence that comprises (or consists of) the complement of a nucleotide sequence that is at least 80% identical to the nucleic acid sequence of SEQ ID NOs: 441 or 444. For example, the reverse primer comprises (or consists of) a nucleotide sequence having at least 80% identity to the nucleic acid sequence of SEQ ID NOs: 441 or 444. The amplified target nucleic acid may be detected using an oligonucleotide probe as described herein (e.g. by reference to SEQ ID NOs: 442 or 445).

In embodiments wherein the one or more biomarker is MYL9, the forward primer hybridises to a target nucleic acid sequence that comprises (or consists of) the complement of the nucleotide sequence of SEQ ID NOs: 446 or 449, or a nucleotide sequence that is at least 80% identical thereto. For example, the forward primer comprises (or consists of) a nucleotide sequence having at least 80% identity to the nucleic acid sequence of SEQ ID NO: 446 or 449. The reverse primer hybridises to a target nucleic acid sequence that comprises (or consists of) the complement of a nucleotide sequence that is at least 80% identical to the nucleic acid sequence of SEQ ID NOs: 447 or 450. For example, the reverse primer comprises (or consists of) a nucleotide sequence having at least 80% identity to the nucleic acid sequence of SEQ ID NOs: 447 or 450. The amplified target nucleic acid may be detected using an oligonucleotide probe as described herein (e.g. by reference to SEQ ID NOs: 448 or 451).

In embodiments wherein the one or more biomarker is TREML1, the forward primer hybridises to a target nucleic acid sequence that comprises (or consists of) the complement of the nucleotide sequence of SEQ ID NOs: 452 or 455, or a nucleotide sequence that is at least 80% identical thereto. For example, the forward primer comprises (or consists of) a nucleotide sequence having at least 80% identity to the nucleic acid sequence of SEQ ID NO: 452 or 455. The reverse primer hybridises to a target nucleic acid sequence that comprises (or consists of) the complement of a nucleotide sequence that is at least 80% identical to the nucleic acid sequence of SEQ ID NOs: 453 or 456. For example, the reverse primer comprises (or consists of) a nucleotide sequence having at least 80% identity to the nucleic acid sequence of SEQ ID NOs: 453 or 456. The amplified target nucleic acid may be detected using an oligonucleotide probe as described herein (e.g. by reference to SEQ ID NOs: 454 or 457).

In embodiments wherein the one or more biomarker is ITGA2B, the forward primer hybridises to a target nucleic acid sequence that comprises (or consists of) the complement of the nucleotide sequence of SEQ ID NOs: 458 or 461, or a nucleotide sequence that is at least 80% identical thereto. For example, the forward primer comprises (or consists of) a nucleotide sequence having at least 80% identity to the nucleic acid sequence of SEQ ID NO: 458 or 461. The reverse primer hybridises to a target nucleic acid sequence that comprises (or consists of) the complement of a nucleotide sequence that is at least 80% identical to the nucleic acid sequence of SEQ ID NOs: 459 or 462. For example, the reverse primer comprises (or consists of) a nucleotide sequence having at least 80% identity to the nucleic acid sequence of SEQ ID NOs: 459 or 462. The amplified target nucleic acid may be detected using an oligonucleotide probe as described herein (e.g. by reference to SEQ ID NOs: 457 or 460).

In embodiments wherein the one or more biomarker is PLA2G7, the forward primer hybridises to a target nucleic acid sequence that comprises (or consists of) the complement of the nucleotide sequence of SEQ ID NOs: 464 or 467, or a nucleotide sequence that is at least 80% identical thereto. For example, the forward primer comprises (or consists of) a nucleotide sequence having at least 80% identity to the nucleic acid sequence of SEQ ID NO: 464 or 467. The reverse primer hybridises to a target nucleic acid sequence that comprises (or consists of) the complement of a nucleotide sequence that is at least 80% identical to the nucleic acid sequence of SEQ ID NOs: 465 or 468. For example, the reverse primer comprises (or consists of) a nucleotide sequence having at least 80% identity to the nucleic acid sequence of SEQ ID NOs: 465 or 468. The amplified target nucleic acid may be detected using an oligonucleotide probe as described herein (e.g. by reference to SEQ ID NOs: 466 or 469).

In embodiments wherein the one or more biomarker is ARHGEF10L, the forward primer hybridises to a target nucleic acid sequence that comprises (or consists of) the complement of the nucleotide sequence of SEQ ID NO: 470, or a nucleotide sequence that is at least 80% identical thereto. For example, the forward primer comprises (or consists of) a nucleotide sequence having at least 80% identity to the nucleic acid sequence of SEQ ID NO: 470. The reverse primer hybridises to a target nucleic acid sequence that comprises (or consists of) the complement of a nucleotide sequence that is at least 80% identical to the nucleic acid sequence of SEQ ID NO: 471. For example, the reverse primer comprises (or consists of) a nucleotide sequence having at least 80% identity to the nucleic acid sequence of SEQ ID NO: 471. The amplified target nucleic acid may be detected using an oligonucleotide probe as described herein (e.g. by reference to SEQ ID NO: 472).

In embodiments wherein the one or more biomarker is TGFB1, the forward primer hybridises to a target nucleic acid sequence that comprises (or consists of) the complement of the nucleotide sequence of SEQ ID NOs: 473 or 476, or a nucleotide sequence that is at least 80% identical thereto. For example, the forward primer comprises (or consists of) a nucleotide sequence having at least 80% identity to the nucleic acid sequence of SEQ ID NO: 473 or 476. The reverse primer hybridises to a target nucleic acid sequence that comprises (or consists of) the complement of a nucleotide sequence that is at least 80% identical to the nucleic acid sequence of SEQ ID NOs: 474 or 477. For example, the reverse primer comprises (or consists of) a nucleotide sequence having at least 80% identity to the nucleic acid sequence of SEQ ID NOs: 474 or 477. The amplified target nucleic acid may be detected using an oligonucleotide probe as described herein (e.g. by reference to SEQ ID NOs: 475 or 478).

In embodiments wherein the one or more biomarker is MYCL, the forward primer hybridises to a target nucleic acid sequence that comprises (or consists of) the complement of the nucleotide sequence of SEQ ID NOs: 479 or 482, or a nucleotide sequence that is at least 80% identical thereto. For example, the forward primer comprises (or consists of) a nucleotide sequence having at least 80% identity to the nucleic acid sequence of SEQ ID NO: 479 or 482. The reverse primer hybridises to a target nucleic acid sequence that comprises (or consists of) the complement of a nucleotide sequence that is at least 80% identical to the nucleic acid sequence of SEQ ID NOs: 480 or 483. For example, the reverse primer comprises (or consists of) a nucleotide sequence having at least 80% identity to the nucleic acid sequence of SEQ ID NOs: 480 or 483. The amplified target nucleic acid may be detected using an oligonucleotide probe as described herein (e.g. by reference to SEQ ID NOs: 481 or 484).

In embodiments wherein the one or more biomarker is CXCR1, the forward primer hybridises to a target nucleic acid sequence that comprises (or consists of) the complement of the nucleotide sequence of SEQ ID NOs: 485 or 488, or a nucleotide sequence that is at least 80% identical thereto. For example, the forward primer comprises (or consists of) a nucleotide sequence having at least 80% identity to the nucleic acid sequence of SEQ ID NO: 485 or 488. The reverse primer hybridises to a target nucleic acid sequence that comprises (or consists of) the complement of a nucleotide sequence that is at least 80% identical to the nucleic acid sequence of SEQ ID NOs: 486 or 489. For example, the reverse primer comprises (or consists of) a nucleotide sequence having at least 80% identity to the nucleic acid sequence of SEQ ID NOs: 486 or 489. The amplified target nucleic acid may be detected using an oligonucleotide probe as described herein (e.g. by reference to SEQ ID NOs: 487 or 490).

In embodiments wherein the one or more biomarker is HCAR2, the forward primer hybridises to a target nucleic acid sequence that comprises (or consists of) the complement of the nucleotide sequence of SEQ ID NOs: 491 or 494, or a nucleotide sequence that is at least 80% identical thereto. For example, the forward primer comprises (or consists of) a nucleotide sequence having at least 80% identity to the nucleic acid sequence of SEQ ID NO: 491 or 494. The reverse primer hybridises to a target nucleic acid sequence that comprises (or consists of) the complement of a nucleotide sequence that is at least 80% identical to the nucleic acid sequence of SEQ ID NOs: 492 or 495. For example, the reverse primer comprises (or consists of) a nucleotide sequence having at least 80% identity to the nucleic acid sequence of SEQ ID NOs: 492 or 495. The amplified target nucleic acid may be detected using an oligonucleotide probe as described herein (e.g. by reference to SEQ ID NOs: 493 or 496).

In embodiments wherein the one or more biomarker is CIQA, the forward primer hybridises to a target nucleic acid sequence that comprises (or consists of) the complement of the nucleotide sequence of SEQ ID NO: 497, or a nucleotide sequence that is at least 80% identical thereto. For example, the forward primer comprises (or consists of) a nucleotide sequence having at least 80% identity to the nucleic acid sequence of SEQ ID NO: 497. The reverse primer hybridises to a target nucleic acid sequence that comprises (or consists of) the complement of a nucleotide sequence that is at least 80% identical to the nucleic acid sequence of SEQ ID NO: 498. For example, the reverse primer comprises (or consists of) a nucleotide sequence having at least 80% identity to the nucleic acid sequence of SEQ ID NO: 498. The amplified target nucleic acid may be detected using an oligonucleotide probe as described herein (e.g. by reference to SEQ ID NO: 499).

In embodiments wherein the one or more biomarker is CIQB, the forward primer hybridises to a target nucleic acid sequence that comprises (or consists of) the complement of the nucleotide sequence of SEQ ID NO: 500, or a nucleotide sequence that is at least 80% identical thereto. For example, the forward primer comprises (or consists of) a nucleotide sequence having at least 80% identity to the nucleic acid sequence of SEQ ID NO: 500. The reverse primer hybridises to a target nucleic acid sequence that comprises (or consists of) the complement of a nucleotide sequence that is at least 80% identical to the nucleic acid sequence of SEQ ID NOs: 501 or 503. For example, the reverse primer comprises (or consists of) a nucleotide sequence having at least 80% identity to the nucleic acid sequence of SEQ ID NOs: 501 or 503. The amplified target nucleic acid may be detected using an oligonucleotide probe as described herein (e.g. by reference to SEQ ID NO: 502).

In embodiments wherein the one or more biomarker is CIQC, the forward primer hybridises to a target nucleic acid sequence that comprises (or consists of) the complement of the nucleotide sequence of SEQ ID NOs: 504 or 507, or a nucleotide sequence that is at least 80% identical thereto. For example, the forward primer comprises (or consists of) a nucleotide sequence having at least 80% identity to the nucleic acid sequence of SEQ ID NOs: 504 or 507. The reverse primer hybridises to a target nucleic acid sequence that comprises (or consists of) the complement of a nucleotide sequence that is at least 80% identical to the nucleic acid sequence of SEQ ID NOs: 505 or 508. For example, the reverse primer comprises (or consists of) a nucleotide sequence having at least 80% identity to the nucleic acid sequence of SEQ ID NOs: 505 or 508. The amplified target nucleic acid may be detected using an oligonucleotide probe as described herein (e.g. by reference to SEQ ID NO: 506 or 509).

In embodiments wherein the one or more biomarker is MRAS, the forward primer hybridises to a target nucleic acid sequence that comprises (or consists of) the complement of the nucleotide sequence of SEQ ID NOs: 510 or 513, or a nucleotide sequence that is at least 80% identical thereto. For example, the forward primer comprises (or consists of) a nucleotide sequence having at least 80% identity to the nucleic acid sequence of SEQ ID NO: 510 or 513. The reverse primer hybridises to a target nucleic acid sequence that comprises (or consists of) the complement of a nucleotide sequence that is at least 80% identical to the nucleic acid sequence of SEQ ID NOs: 511 or 514. For example, the reverse primer comprises (or consists of) a nucleotide sequence having at least 80% identity to the nucleic acid sequence of SEQ ID NOs: 511 or 514. The amplified target nucleic acid may be detected using an oligonucleotide probe as described herein (e.g. by reference to SEQ ID NOs: 512 or 515).

In embodiments wherein the one or more biomarker is SLC39A8, the forward primer hybridises to a target nucleic acid sequence that comprises (or consists of) the complement of the nucleotide sequence of SEQ ID NOs: 516 or 519, or a nucleotide sequence that is at least 80% identical thereto. For example, the forward primer comprises (or consists of) a nucleotide sequence having at least 80% identity to the nucleic acid sequence of SEQ ID NO: 516 or 519. The reverse primer hybridises to a target nucleic acid sequence that comprises (or consists of) the complement of a nucleotide sequence that is at least 80% identical to the nucleic acid sequence of SEQ ID NOs: 517 or 520. For example, the reverse primer comprises (or consists of) a nucleotide sequence having at least 80% identity to the nucleic acid sequence of SEQ ID NOs: 517 or 520. The amplified target nucleic acid may be detected using an oligonucleotide probe as described herein (e.g. by reference to SEQ ID NOs: 518 or 521).

In embodiments wherein the one or more biomarker is TMEM37, the forward primer hybridises to a target nucleic acid sequence that comprises (or consists of) the complement of the nucleotide sequence of SEQ ID NO: 522, or a nucleotide sequence that is at least 80% identical thereto. For example, the forward primer comprises (or consists of) a nucleotide sequence having at least 80% identity to the nucleic acid sequence of SEQ ID NO: 522. The reverse primer hybridises to a target nucleic acid sequence that comprises (or consists of) the complement of a nucleotide sequence that is at least 80% identical to the nucleic acid sequence of SEQ ID NO: 523. For example, the reverse primer comprises (or consists of) a nucleotide sequence having at least 80% identity to the nucleic acid sequence of SEQ ID NO: 523. The amplified target nucleic acid may be detected using an oligonucleotide probe as described herein (e.g. by reference to SEQ ID NOs: 524 or 525).

In embodiments wherein the one or more biomarker is FCER1A, the forward primer hybridises to a target nucleic acid sequence that comprises (or consists of) the complement of the nucleotide sequence of SEQ ID NOs: 526 or 529, or a nucleotide sequence that is at least 80% identical thereto. For example, the forward primer comprises (or consists of) a nucleotide sequence having at least 80% identity to the nucleic acid sequence of SEQ ID NO: 526 or 529. The reverse primer hybridises to a target nucleic acid sequence that comprises (or consists of) the complement of a nucleotide sequence that is at least 80% identical to the nucleic acid sequence of SEQ ID NOs: 527 or 530. For example, the reverse primer comprises (or consists of) a nucleotide sequence having at least 80% identity to the nucleic acid sequence of SEQ ID NOs: 527 or 530. The amplified target nucleic acid may be detected using an oligonucleotide probe as described herein (e.g. by reference to SEQ ID NOs: 528 or 531).

In embodiments wherein the one or more biomarker is BLC11B, the forward primer hybridises to a target nucleic acid sequence that comprises (or consists of) the complement of the nucleotide sequence of SEQ ID NOs: 532 or 535, or a nucleotide sequence that is at least 80% identical thereto. For example, the forward primer comprises (or consists of) a nucleotide sequence having at least 80% identity to the nucleic acid sequence of SEQ ID NO: 532 or 535. The reverse primer hybridises to a target nucleic acid sequence that comprises (or consists of) the complement of a nucleotide sequence that is at least 80% identical to the nucleic acid sequence of SEQ ID NOs: 533 or 536. For example, the reverse primer comprises (or consists of) a nucleotide sequence having at least 80% identity to the nucleic acid sequence of SEQ ID NOs: 533 or 536. The amplified target nucleic acid may be detected using an oligonucleotide probe as described herein (e.g. by reference to SEQ ID NOs: 534 or 537).

In embodiments wherein the one or more biomarker is PKHD1, the forward primer hybridises to a target nucleic acid sequence that comprises (or consists of) the complement of the nucleotide sequence of SEQ ID NOs: 538 or 541, or a nucleotide sequence that is at least 80% identical thereto. For example, the forward primer comprises (or consists of) a nucleotide sequence having at least 80% identity to the nucleic acid sequence of SEQ ID NO: 538 or 541. The reverse primer hybridises to a target nucleic acid sequence that comprises (or consists of) the complement of a nucleotide sequence that is at least 80% identical to the nucleic acid sequence of SEQ ID NOs: 539 or 542. For example, the reverse primer comprises (or consists of) a nucleotide sequence having at least 80% identity to the nucleic acid sequence of SEQ ID NOs: 539 or 542. The amplified target nucleic acid may be detected using an oligonucleotide probe as described herein (e.g. by reference to SEQ ID NOs: 540 or 543).

In embodiments wherein the one or more biomarker is KLRB1, the forward primer hybridises to a target nucleic acid sequence that comprises (or consists of) the complement of the nucleotide sequence of SEQ ID NOs: 544 or 547, or a nucleotide sequence that is at least 80% identical thereto. For example, the forward primer comprises (or consists of) a nucleotide sequence having at least 80% identity to the nucleic acid sequence of SEQ ID NO: 544 or 547. The reverse primer hybridises to a target nucleic acid sequence that comprises (or consists of) the complement of a nucleotide sequence that is at least 80% identical to the nucleic acid sequence of SEQ ID NOs: 545 or 548. For example, the reverse primer comprises (or consists of) a nucleotide sequence having at least 80% identity to the nucleic acid sequence of SEQ ID NOs: 545 or 548. The amplified target nucleic acid may be detected using an oligonucleotide probe as described herein (e.g. by reference to SEQ ID NOs: 546 or 549).

In embodiments wherein the one or more biomarker is LILRB5, the forward primer hybridises to a target nucleic acid sequence that comprises (or consists of) the complement of the nucleotide sequence of SEQ ID NOs: 550 or 553, or a nucleotide sequence that is at least 80% identical thereto. For example, the forward primer comprises (or consists of) a nucleotide sequence having at least 80% identity to the nucleic acid sequence of SEQ ID NO: 550 or 553. The reverse primer hybridises to a target nucleic acid sequence that comprises (or consists of) the complement of a nucleotide sequence that is at least 80% identical to the nucleic acid sequence of SEQ ID NOs: 551 or 554. For example, the reverse primer comprises (or consists of) a nucleotide sequence having at least 80% identity to the nucleic acid sequence of SEQ ID NOs: 551 or 554. The amplified target nucleic acid may be detected using an oligonucleotide probe as described herein (e.g. by reference to SEQ ID NOs: 552 or 555).

In embodiments wherein the one or more biomarker is NECAB1, the forward primer hybridises to a target nucleic acid sequence that comprises (or consists of) the complement of the nucleotide sequence of SEQ ID NOs: 556 or 559, or a nucleotide sequence that is at least 80% identical thereto. For example, the forward primer comprises (or consists of) a nucleotide sequence having at least 80% identity to the nucleic acid sequence of SEQ ID NO: 556 or 559. The reverse primer hybridises to a target nucleic acid sequence that comprises (or consists of) the complement of a nucleotide sequence that is at least 80% identical to the nucleic acid sequence of SEQ ID NOs: 557 or 560. For example, the reverse primer comprises (or consists of) a nucleotide sequence having at least 80% identity to the nucleic acid sequence of SEQ ID NOs: 557 or 560. The amplified target nucleic acid may be detected using an oligonucleotide probe as described herein (e.g. by reference to SEQ ID NOs: 558 or 561).

In embodiments wherein the one or more biomarker is NECAB2, the forward primer hybridises to a target nucleic acid sequence that comprises (or consists of) the complement of the nucleotide sequence of SEQ ID NOs: 562 or 565, or a nucleotide sequence that is at least 80% identical thereto. For example, the forward primer comprises (or consists of) a nucleotide sequence having at least 80% identity to the nucleic acid sequence of SEQ ID NO: 562 or 565. The reverse primer hybridises to a target nucleic acid sequence that comprises (or consists of) the complement of a nucleotide sequence that is at least 80% identical to the nucleic acid sequence of SEQ ID NOs: 563 or 566. For example, the reverse primer comprises (or consists of) a nucleotide sequence having at least 80% identity to SEQ ID NOs: 563 or 566. The amplified target nucleic acid may be detected using an oligonucleotide probe as described herein (e.g. by reference to SEQ ID NOs: 564 or 567).

In embodiments wherein the control gene is ALAS1, the forward primer comprises (or consists of) a nucleotide sequence having at least 80% identity to the nucleic acid sequence of SEQ ID NO: 568 or 571. The reverse primer comprises (or consists of) a nucleotide sequence having at least 80% identity to SEQ ID NOs: 569 or 572. The amplified target nucleic acid may be detected using an oligonucleotide probe that comprises a nucleic acid sequence having at least 80% sequence identity to the nucleic acid sequence of SEQ ID NOs: 570 or 573.

In embodiments wherein the control gene is HMBS, the forward primer comprises (or consists of) a nucleotide sequence having at least 80% identity to the nucleic acid sequence of SEQ ID NO: 574 or 577. The reverse primer comprises (or consists of) a nucleotide sequence having at least 80% identity to SEQ ID NOs: 575 or 578. The amplified target nucleic acid may be detected using an oligonucleotide probe that comprises a nucleic acid sequence having at least 80% sequence identity to the nucleic acid sequence of SEQ ID NOs: 576 or 579.

In embodiments wherein the control gene is GTF2D1, the forward primer comprises (or consists of) a nucleotide sequence having at least 80% identity to the nucleic acid sequence of SEQ ID NO: 580 or 583. The reverse primer comprises (or consists of) a nucleotide sequence having at least 80% identity to SEQ ID NOs: 581 or 584. The amplified target nucleic acid may be detected using an oligonucleotide probe that comprises a nucleic acid sequence having at least 80% sequence identity to the nucleic acid sequence of SEQ ID NOs: 582 or 585.

In all of the methods and uses described herein, the presence and/or amount of the one or more biomarker may be determined using a forward oligonucleotide primer and/or reverse oligonucleotide primer specific for the one or more biomarker to amplify a target nucleic acid sequence. The forward and reverse oligonucleotide primers used in the methods and uses of the invention are as described herein.

### Kits and Devices

The present invention also provides kits and devices that are useful in diagnosing a systemic inflammatory condition (including diagnosing SIRS, sepsis, abdominal sepsis, and pulmonary sepsis), distinguishing between sepsis and SIRS, distinguishing between abdominal sepsis and pulmonary sepsis, monitoring of a systemic inflammatory condition (including monitoring of SIRS, sepsis, abdominal sepsis, and pulmonary sepsis), determining whether a patient is suitable for discharge from medical care, and diagnosing organ damage.

The kits and devices of the present invention comprise one or more biomarker of the invention and/or one or more agent for the detection of or for the determination of the amount of the one or more biomarker of the invention. For example, the kit and device may comprise one or more biomarker of the invention. For example, the kit and device may comprise one or more agent for the detection of or for the determination of the amount of the one or more biomarker of the invention. The “one or more agent” may comprise one or more binding agent specific for the one or more biomarker.

The “one or more biomarker of the invention” may be as described herein. Specific biomarkers and agents for the detection of said biomarkers useful in the present invention are set forth herein. The biomarkers of the kit or device can be used to generate biomarker profiles according to the present invention.

In one embodiment, the kit and device of the present invention may comprise one or more inflammation biomarker of the invention (e.g. as described herein) and/or one or more agent for the detection of or for the determination of the amount of the one or more inflammation biomarker of the invention. For example, the “one or more inflammation biomarker of the invention” may comprise one or more (e.g. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, or all 21) inflammation biomarker selected from the group consisting of: FAM20A, OLAH, CD177, ADM, IL10, METTL7B, MMP9, RETN, TDRD9, ITGA7, BMX, HP, IGFBP2, ALPL, DACH1, IL1R1, IL1R2, CYP19A1, MMP8, TGFA and VSTM1. For example, the “one or more inflammation biomarker of the invention” may comprise one of more (2 or more, or all 3) of: FAM20A, OLAH, and CD177. For example, the “one or more inflammation biomarker of the invention” may comprise FAM20A and OLAH.

In one embodiment, the kit and device of the present invention may comprise one or more sepsis biomarker of the invention and/or one or more agent for the detection of or for the determination of the amount of the one or more sepsis biomarker of the invention. For example, the “one or more sepsis biomarker of the invention” may comprise one or more (e.g. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or all 30) sepsis biomarker selected from the group consisting of: ITGB3, ITGA2B, MYL9, LCN2, TREML1, LCN15, CMTM5, PPBP, PF4, KIF2C, MAP1A, SELP, NEXN, NLRC4, CLEC1B, SLC39A8, CIQC, CIQA, MRAS, TMEM37, CIQB, PCOLCE2, KIF2C, TNF, IF144, IFIT1, RPGRIP1, HCAR2, CXCR1, DISC1, and EPSTI1. For example, the “one or more sepsis biomarker of the invention” may comprise one of more (2 or more, 3 or more, 4 or more, 5 or more, 6 or more, 7 or more, 8 or more or all 9) of ITGB3, ITGA2B, MYL9, LCN2, TREML1, LCN15, CMTM5, PPBP, and PF4. For example, the “one or more sepsis biomarker of the invention” may comprise one of more (2 or more, 3 or more, 4 or more, or all 5) of the sepsis biomarkers ITGB3, ITGA2B, MYL9, LCN2, and TREML1.

In one embodiment, the kit and device of the present invention may comprise one or more SIRS biomarker of the invention and/or one or more agent for the detection of or for the determination of the amount of the one or more SIRS biomarker of the invention. For example, the “one or more SIRS biomarker of the invention” may comprise one or more (e.g. 2 or more, 3 or more, 4 or more, 5 or more, 6 or more, 7 or more, 8 or more, or all 9) SIRS biomarker selected from the group consisting of: of PLA2G7, ARHGEF10L, MYCL, TGFBI, and GPR124, IL1 RN, NLRP3, RBP4, and MPP3. For example, the “one or more SIRS biomarker of the invention” may comprise one of more (2 or more, 3 or more, 4 or more, or all 5) of PLA2G7, ARHGEF10L, MYCL, TGFBI, and GPR124. For example, the “one or more SIRS biomarker of the invention” may comprise one of more (2 or more, 3 or more, or all 4) of PLA2G7, ARHGEF10L, MYCL, and TGFBI.

In one embodiment, the kit and device of the present invention may comprise:
(i) one or more inflammation biomarker (as described herein),
(ii) one or more sepsis marker (as described herein), and/or
(iii) one or more SIRS biomarker (as described herein);
   and/or one or more agent for the detection of or for the determination of the amount of the one or more biomarker.

For example, the kit and device may comprise one or more agent for the detection of or for the determination of the amount of: (i) one or more inflammation biomarker (as described herein), (ii) one or more sepsis marker (as described herein), and/or (iii) one or more SIRS biomarker (as described herein). For example, the kit and device may comprise one or more agent for the detection of or for the determination of the amount of: (i) one of more (2 or more, or all 3) of the inflammatory biomarker selected from the group consisting of: FAM20A, OLAH, and optionally CD177; (ii) one of more (2 or more, 3 or more, 4 or more, or all 5) of the sepsis biomarker selected from the group consisting of: ITGB3, ITGA2B, MYL9, LCN2, and TREML1; and/or (iii) one of more (2 or more, 3 or more, or all 4) of the SIRS biomarker selected from the group consisting of: PLA2G7, ARHGEF10L, MYCL, and TGFBI.

In one embodiment, the kit and device of the present invention may comprise one or more abdominal sepsis biomarker of the invention and/or one or more agent for the detection of or for the determination of the amount of the one or more abdominal sepsis biomarker of the invention. For example, the “one or more abdominal sepsis biomarker of the invention” may comprise one or more (e.g. 2 or more, 3 or more, 4 or more, 5 or more, 6 or more, 7 or more, 8 or more, 9 or more, 10 or more, 11 or more or all 12) abdominal sepsis biomarker selected from the group consisting of: SLC39A8, CIQC, CIQA, MRAS, TMEM37, CIQB, PCOLCE2, KIF2C, TNF, IF144, IFIT1, and RPGRIP1. For example, the “one or more abdominal sepsis biomarker of the invention” may comprise one or more (e.g. 2 or more, 3 or more, 4 or more, 5 or more, or all 6) of SLC39A8, CIQC, CIQA, MRAS, TMEM37, CIQB. For example, the “one or more abdominal sepsis biomarker of the invention” may comprise one of more (2 or more, or all 3) of SLC39A8, CIQC, CIQA.

In one embodiment, the kit and device of the present invention may comprise one or more pulmonary sepsis biomarker of the invention and/or one or more agent for the detection of or for the determination of the amount of the one or more pulmonary sepsis biomarker of the invention. For example, the “one or more pulmonary sepsis biomarker of the invention” may comprise one or more (e.g. 2 or more, 3 or more, 4 or more, or all 5) pulmonary sepsis biomarker selected from the group consisting of: HCAR2, CXCR1, DISC1, EPSTI1, and IF144. For example, the “one or more pulmonary sepsis biomarker of the invention” may comprise one of more (2 or more, or all 3) of HCAR2, CXCR1, and DISC1.

In one embodiment, the kit and device of the present invention may comprise:
(i) one or more abdominal sepsis biomarker (as described herein), and/or
(ii) one or more pulmonary sepsis biomarker (as described herein),
   and/or one or more agent for the detection of or for the determination of the amount of the one or more biomarker.

For example, the kit and device may comprise one or more agent for the detection of or for the determination of the amount of: (i) one or more abdominal sepsis biomarker (as described herein); and/or (ii) one or more pulmonary sepsis marker (as described herein). For example, the kit and device may comprise one or more agent for the detection of or for the determination of the amount of: (i) one or more (2 or more, 3 or more, 4 or more, 5 or more, or all 6) of the abdominal sepsis biomarker selected from the group consisting of: SLC39A8, CIQC, CIQA, MRAS, TMEM37, and CIQB; and/or (ii) one or more (e.g. 2 or more, or all 3) of the pulmonary sepsis biomarker selected from the group consisting of: HCAR2, CXCR1, and DISC1;

In one embodiment, the kit and device of the present invention may comprise one or more prognosis biomarker of the invention and/or one or more agent for the detection of or for the determination of the amount of the one or more recovery biomarker of the invention. For example, the “one or more prognosis biomarker of the invention” may comprise one or more (e.g. 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or all 20) biomarker selected from the group consisting of: ITM2A, CCL5, NPPC, PKD1, KLRK1, KLRB1, HLA-DRA, BCL11B, HLA-DPB1, FCER1A, DAAM2, SLAMF6, CD160, KLRF1, CD2, LGALS2, MYCL, MX1, NECAB1, and PKHD1. For example, the “one or more biomarker of the invention” may comprise one of more (e.g. 2 or more, 3 or more, 4 or more, 5 or more, 6 or more, 7 or more, 8 or more, 9 or more, 10 or more, 11 or more, or all 12) of ITM2A, CCL5, NPPC, KLRK1, KLRB1, HLA-DRA, BCL11B, HLA-DPB1, SLAMF6, CD160, KLRF1, and MX1. For example, the “one or more biomarker of the invention” may comprise one of more (e.g. 2 or more, 3 or more, 4 or more, 5 or more, 6 or more, or all 7) of CCL5, NPPC, PKD1, LGALS2, MYCL, NECAB1, and PKHD1.

In one embodiment, the kit and device of the present invention may comprise one or more survival biomarker of the invention and/or one or more agent for the detection of or for the determination of the amount of the one or more survival biomarker of the invention. For example, the “one or more survival biomarker of the invention” may comprise one or more (e.g. 2 or more, 3 or more, 4 or more, 5 or more, or all 6) survival biomarker selected from the group consisting of: NECAB1, NECAB2 PKDI, PKHD1, LILRB4, and LILRB5. For example, the “one or more survival biomarker of the invention” may comprise one or more (e.g. 2 or more, 3 or more, of all 4) of NECAB1, PKDI, PKHD1, and LILRB5. For example, the “one or more survival biomarker of the invention” may comprise NECAB2 and/or PKD1. For example, the “one or more survival biomarker of the invention” may comprise PKHD1 and/or LILRB5.

The kit and device of the present invention may comprise:
(i) one or more prognosis biomarker (as described herein), and/or
(ii) one or more survival biomarker (as described herein),
and/or one or more agent for the detection of or for the determination of the amount of the one or more biomarker.

For example, the kit and device may comprise one or more agent for the detection of or for the determination of the amount of: (i) one or more prognosis biomarker (as described herein); and/or (ii) one or more survival biomarker (as described herein).

In one embodiment, the kit and device of the present invention may comprise one or more agent for the detection of or for the determination of the amount of:
(i) one or more inflammation biomarker (as described herein),
(ii) one or more sepsis biomarker (as described herein),
(iii) one or more abdominal sepsis biomarker (as described herein),
(iv) one or more pulmonary sepsis biomarker (as described herein),
(v) one or more SIRS biomarker (as described herein),
(vi) one or more prognosis biomarker (as described herein), and/or
(vii) one or more survival biomarker (as described herein).

Generally, the biomarkers and agents of the kit or device will bind, with at least some specificity, to the biomarker molecules contained in the sample from which the biomarker profile is generated. In one embodiment, the kit or device of the invention comprises one or more binding agent specific for the one or more biomarker. Examples of classes of compounds of the kit or device include, but are not limited to, proteins (including antibodies), and fragments thereof, peptides, polypeptides, proteoglycans, glycoproteins, lipoproteins, carbohydrates, lipids, nucleic acids, organic and inorganic chemicals, and natural and synthetic polymers. The biomarker(s) and/or agent(s) for the detection of the one or more biomarker may be part of an array, or the biomarker(s) and/or agent(s) may be packaged separately and/or individually. The biomarker(s) and/or agent(s) may be immobilised on an inert support. The biomarkers(s) and/or agent(s) may be immobilised on a surface to provide a binding agent array.

The device may be a lateral flow device. Lateral flow devices and methods for their construction are well known in the art, being best known as the standard pregnancy test kit. The device may be portable. The device may be disposable. The device may be suitable for use as a point of care diagnostic test. The device may be suitable for use in the home or in the clinic.

The kit or device may also comprise at least one internal standard to be used in generating the biomarker profiles of the present invention. Likewise, the internal standards can be any of the classes of compounds described above.

The kits and devices of the present invention also may contain reagents that can be used to detectably label biomarkers contained in the biological samples from which the biomarker profiles are generated. For this purpose, the kit or device may comprise a set of antibodies or functional fragments thereof that specifically bind at least two, three, four, five, 10, 20, 30, 40, 50 or more, up to all of the biomarkers set forth in any one of Tables 1 to 4 that list biomarkers for use in the invention. The antibodies themselves may be detectably labelled. The kit or device also may comprise a specific biomarker binding component, such as an aptamer.

In a preferred embodiment, a kit or device of the invention comprises (i) one or more antibody specific for the one or more biomarkers of the invention; and/or (ii) one or more oligonucleotide specific for the one or more biomarker of the invention. For example, the one or more oligonucleotide specific for the one or more biomarker is an oligonucleotide of the invention, preferably one or more of SEQ ID NOs: 86-585.

If the biomarkers comprise a nucleic acid, the kit or device may provide one or more oligonucleotide probe that is capable of forming a duplex with the one or more biomarker or with a complementary strand of said one or more biomarker. The one or more oligonucleotide probe may be detectably labelled. Typically, the one or more oligonucleotide probe used in the methods of the invention is selected from one or more of the oligonucleotide probe described herein.

In one embodiment, the one or more oligonucleotide probe is selected from an oligonucleotide probe that comprises or is complementary to at least one nucleic acid sequence having at least 80% sequence identity to the nucleic acid sequence of any one or more of SEQ ID NOs: 86-421. The oligonucleotide probe(s) may be bound to a solid support (such as a microarray).

In one embodiment, the one or more oligonucleotide probe is selected from an oligonucleotide probe that comprises or is complementary to at least one nucleic acid sequence having at least 80% sequence identity to the nucleic acid sequences of any one or more of SEQ ID NOs: 424, 427, 430, 433, 436, 439, 442, 445, 448, 451, 454, 457, 460, 463, 466, 469, 472, 475, 478, 481, 484, 487, 490, 493, 496, 499, 502, 506, 509, 512, 515, 518, 521, 524, 525, 528, 531, 534, 537, 540, 543, 546, 549, 552, 555, 558, 561, 564, 567, 570, 573, 576, 579, 582, and 585 (as described in Table 15).

The kit or device may further comprise: (i) one or more forward oligonucleotide primer; and/or (ii) one or more reverse oligonucleotide primer for amplification of the target nucleic acid sequence. The forward and reverse oligonucleotide primers may be as described herein (and exemplified in Table 15). The amplification product may be detected using the corresponding oligonucleotide probe(s) described herein (as exemplified in Table 15). The kit or device may further comprise one or more reagent for performing quantitative PCR.

The kits and devices of the present invention may also include pharmaceutical excipients, diluents and/or adjuvants when the biomarker is to be used to raise an antibody. Examples of pharmaceutical adjuvants include, but are not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

The present invention is discussed in more detail by means of the Examples described below, and by the Figures.

### FIGURES

FIG. 1: shows a plot of normalised gene expression in patients having SIRS, abdominal sepsis and pulmonary sepsis for the biomarkers identified as being associated with systemic inflammatory conditions (see Table 1). Data is included for the patients that survive (“Survived”) and the patients that did not survive (“Died”) and relates to samples taken at ‘day 1’, ‘day 2’ and ‘day 5’ post-hospitalisation (to the ICU). Data is also included for healthy control patients (shown on the far left-hand side of the plot). Data points are mean expression across all individuals, depicted with standard error bars.

FIG. 2: shows a plot of normalised gene expression in patients having SIRS, abdominal sepsis and pulmonary sepsis for the biomarkers identified as being associated with SIRS (see Table 2). The data shown in the plot is as described for FIG. 1.

FIG. 3: shows a plot of normalised gene expression in patients having SIRS, abdominal sepsis and pulmonary sepsis for the biomarkers identified as being associated with sepsis (see Table 3). The data shown in the plot is as described for FIG. 1.

FIG. 4: shows a plot of normalised gene expression in patients having SIRS, abdominal sepsis and pulmonary sepsis for the biomarkers identified as being associated with prognosis of recovery from a systemic inflammatory condition (see Tables 1 and 4). The data shown in the plot is as described for FIG. 1.

FIG. 5: provides the results from ROC analysis of the gene expression data for the inflammation biomarkers when comparing healthy controls to disease patients.

FIG. 6: provides the results from ROC analysis of the gene expression data for the sepsis and SIRS biomarkers when comparing patients having sepsis to patients having SIRS.

FIG. 7: provides the results from ROC analysis of the gene expression data for the abdominal sepsis and pulmonary sepsis biomarkers when comparing patients having abdominal sepsis to patients having pulmonary sepsis.

FIG. 8: provides the results from ROC analysis of the gene expression data for the prognosis biomarker PKHD1, when comparing healthy controls to disease patients that survived SIRS.

FIG. 9: provides the results from ROC analysis of the gene expression data for the survival biomarkers when comparing disease patients that survived to disease patients that died.

FIG. 10: provides the results from ROC analysis of protein quantification data.

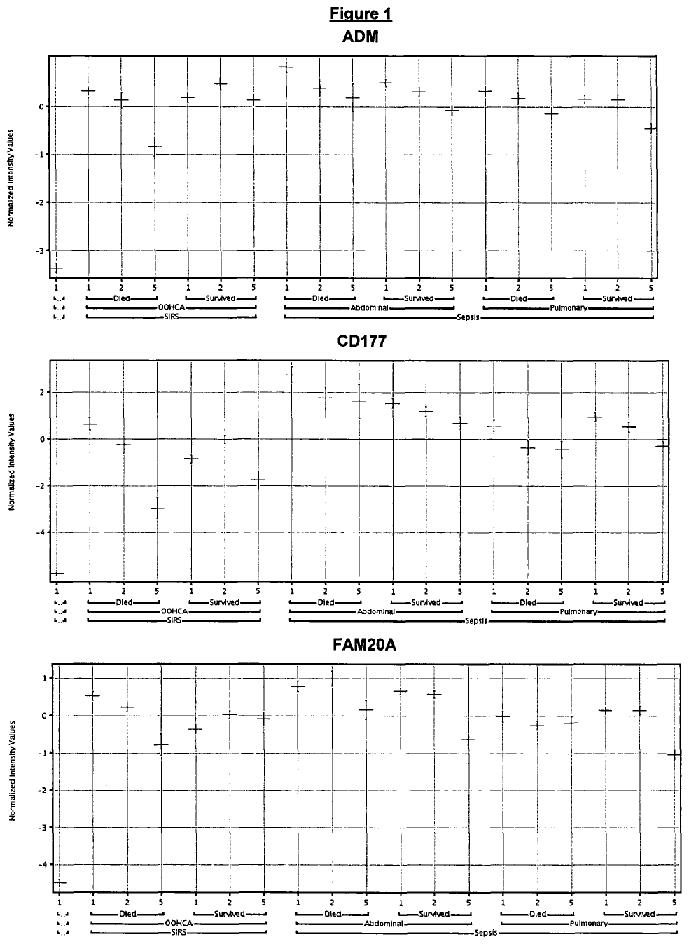

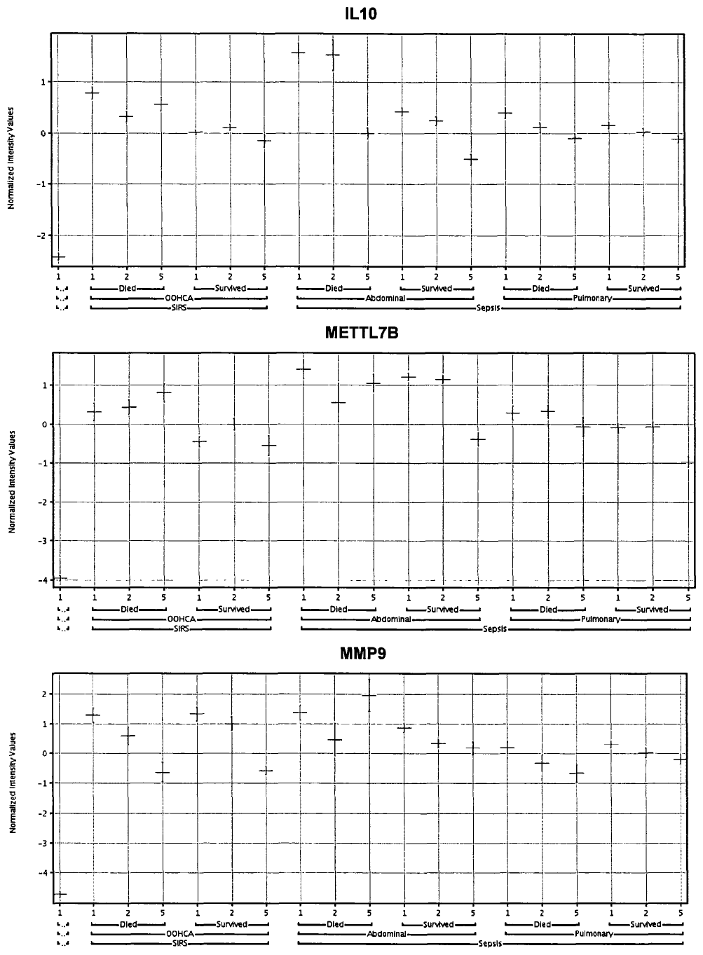

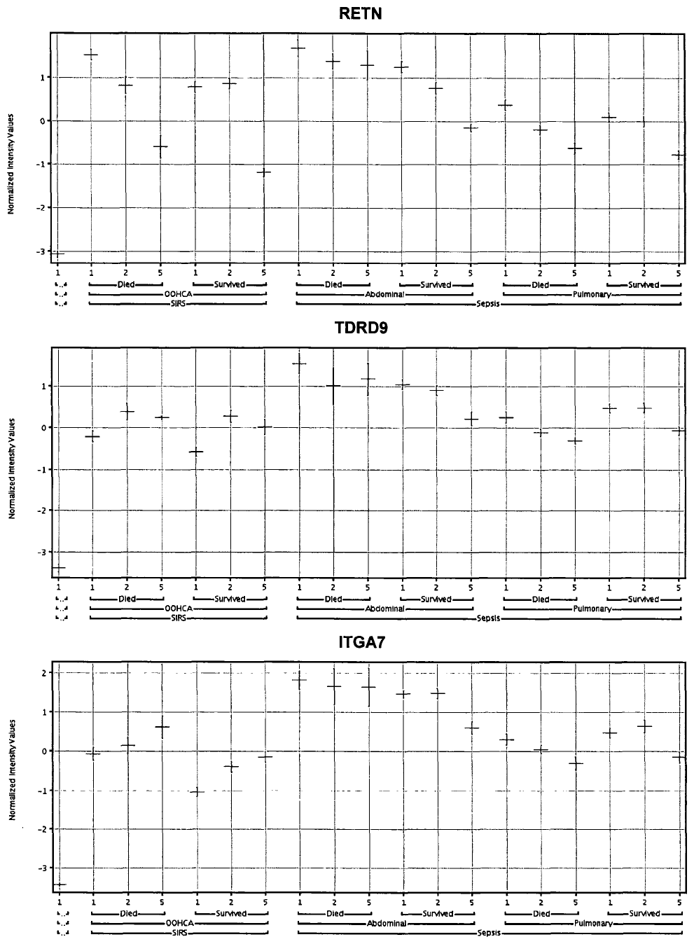

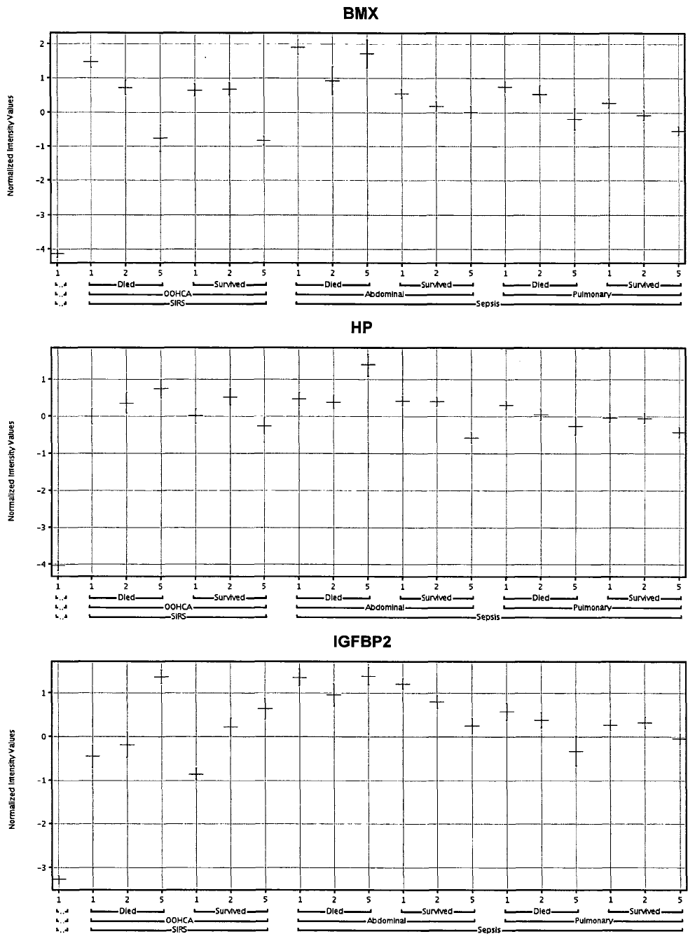

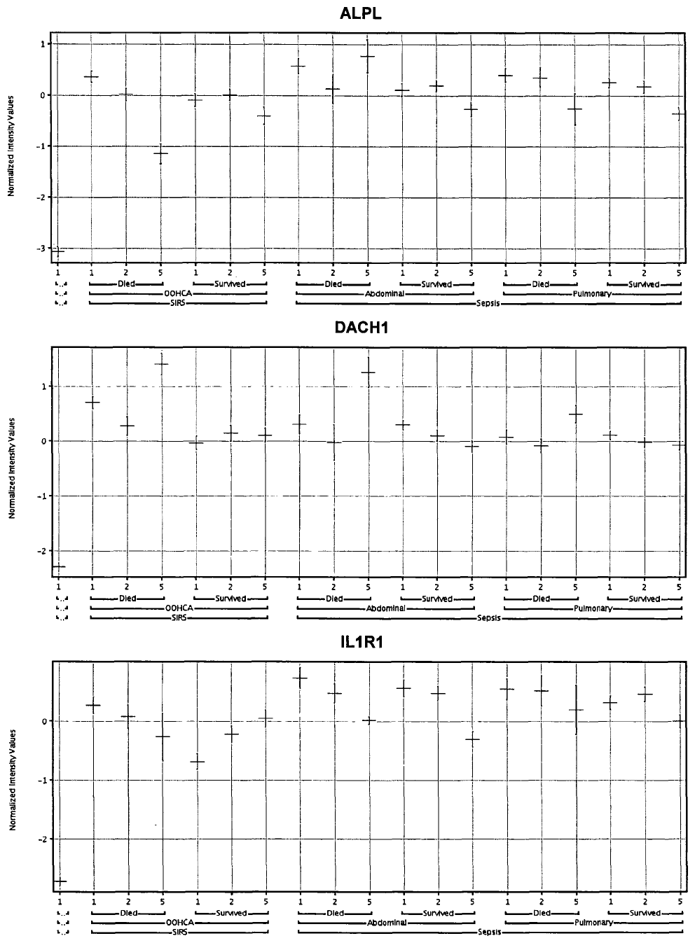

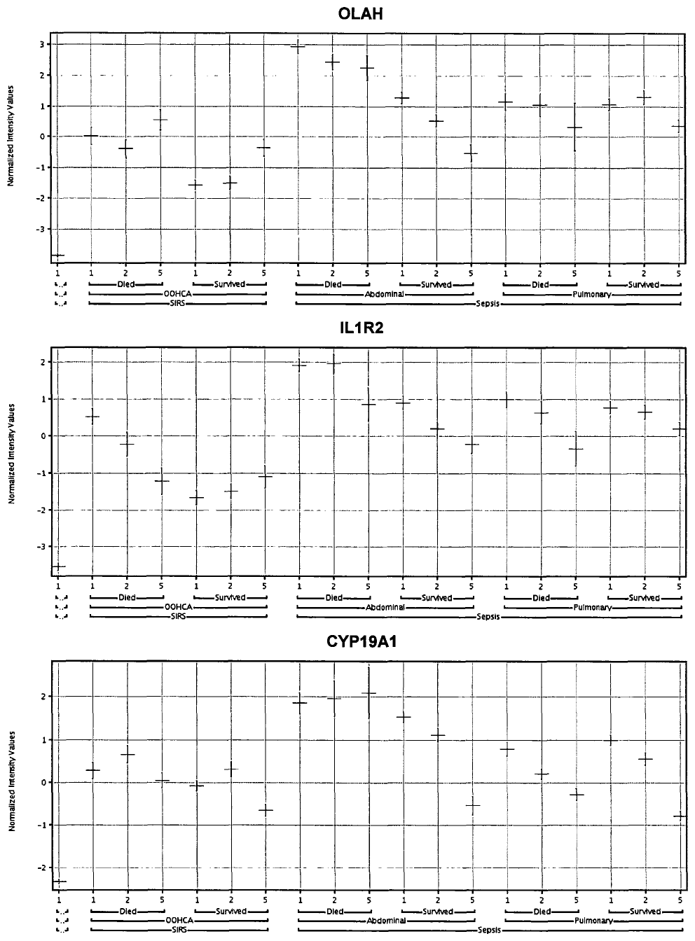

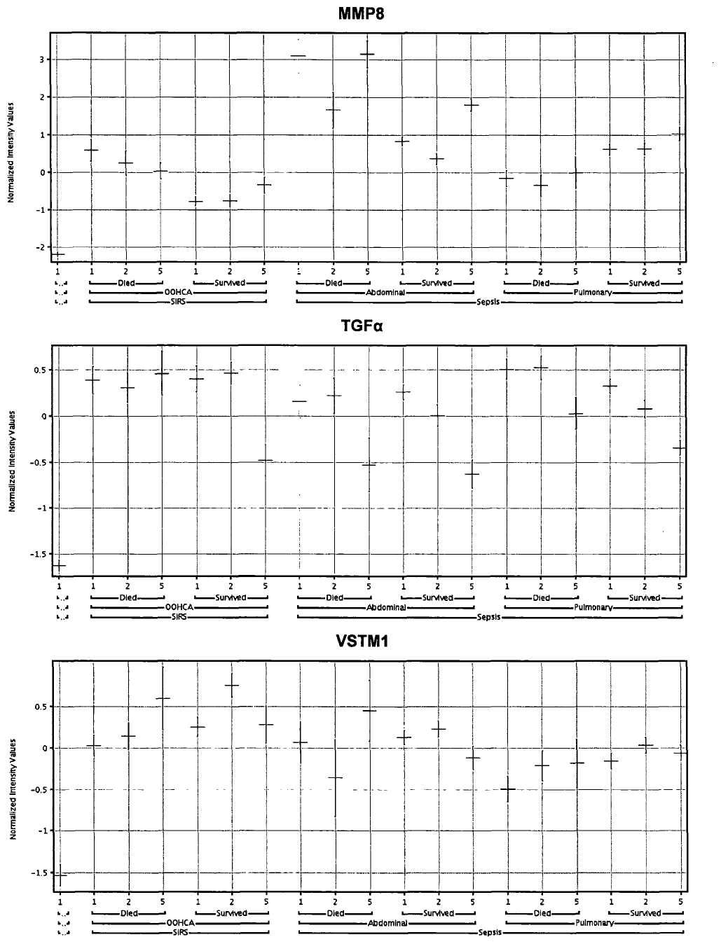

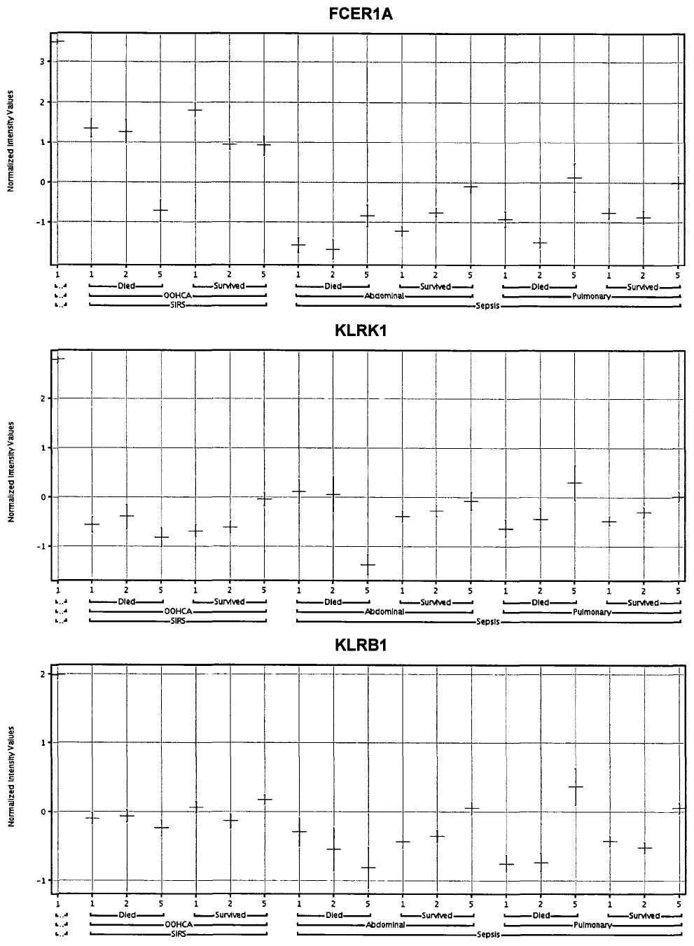

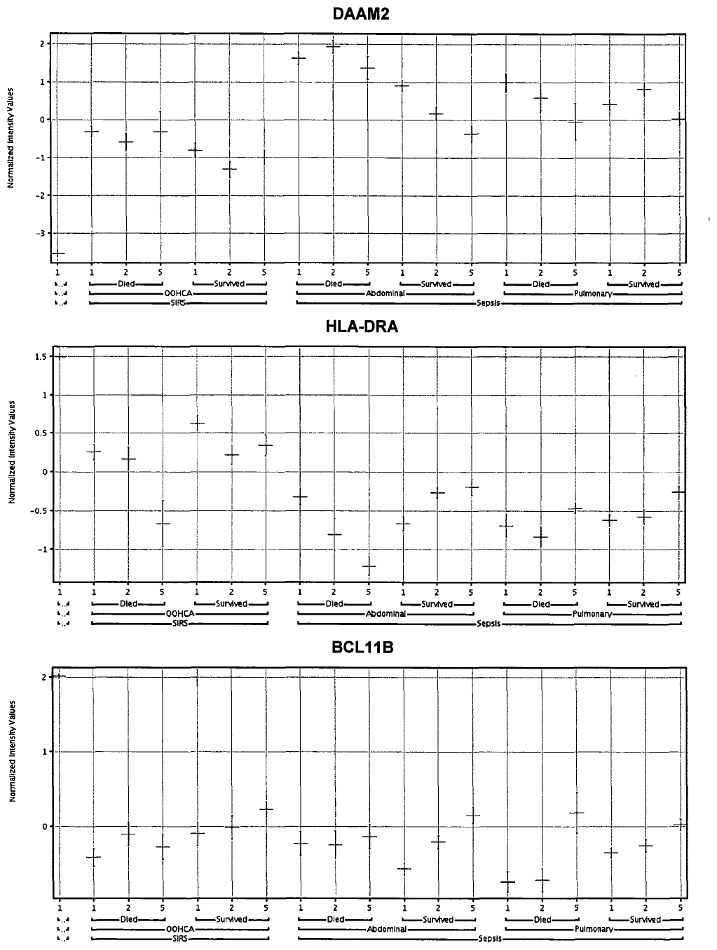

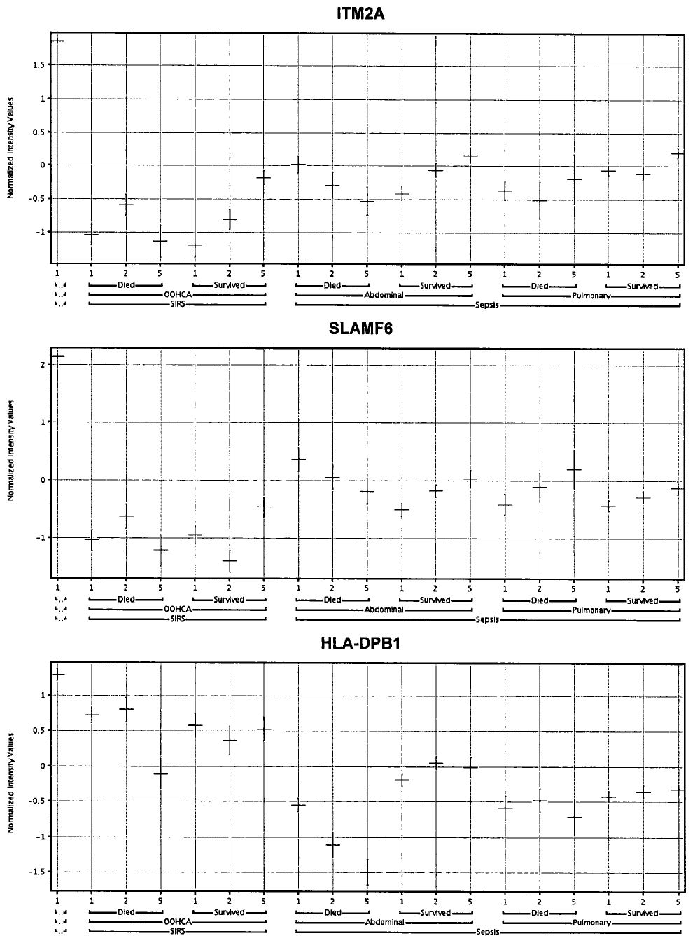

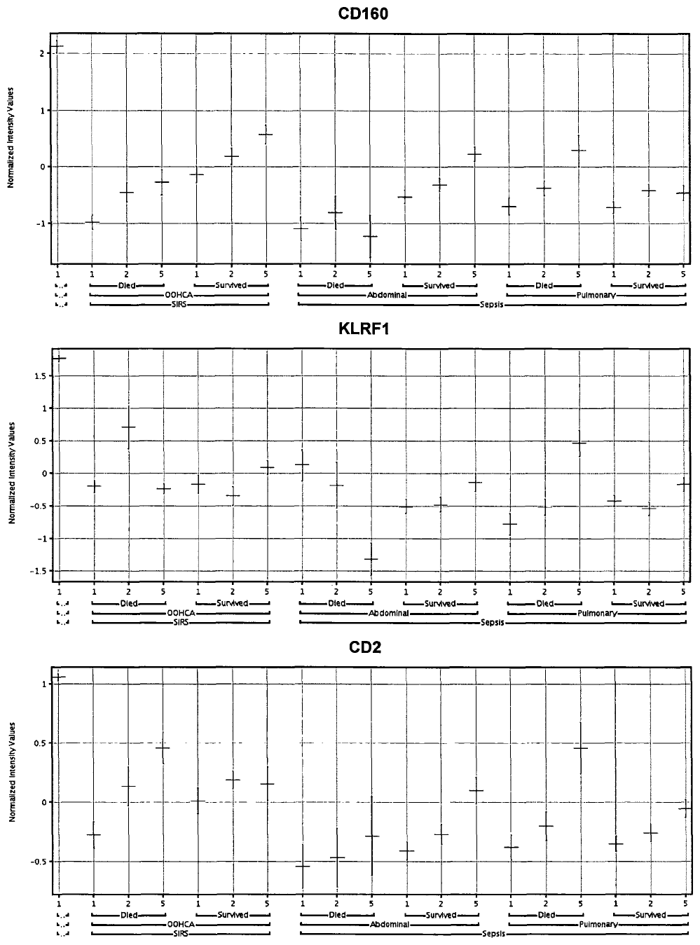

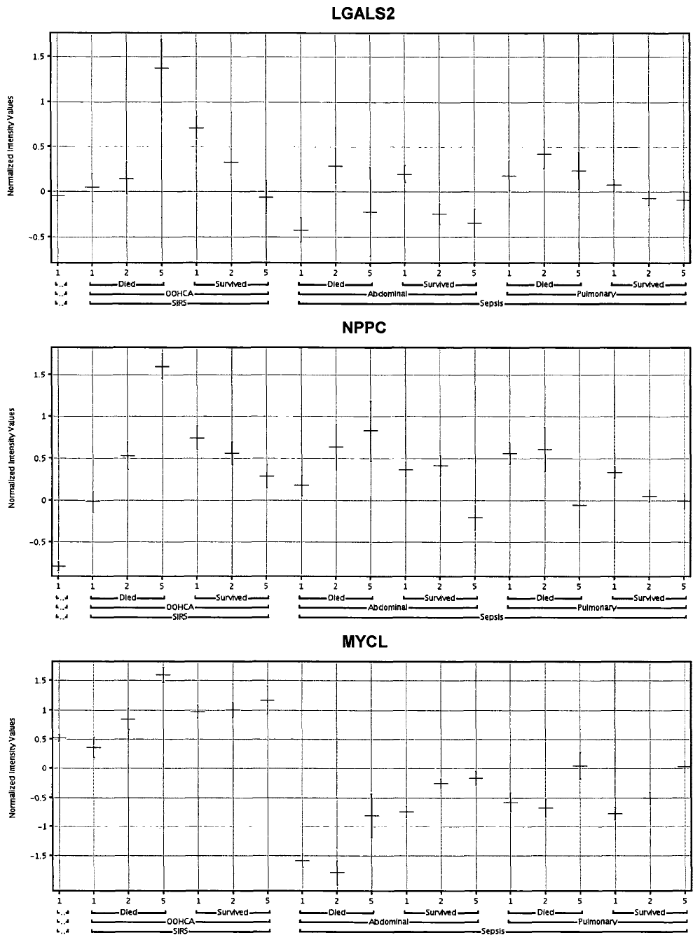

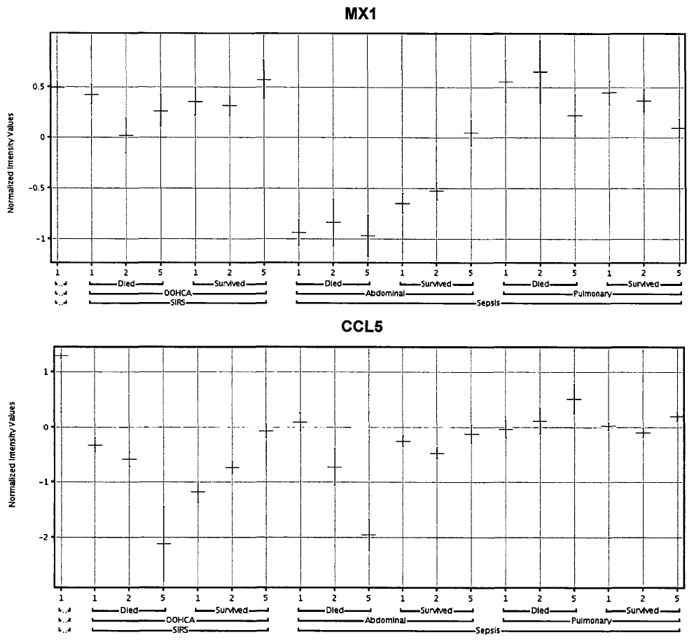

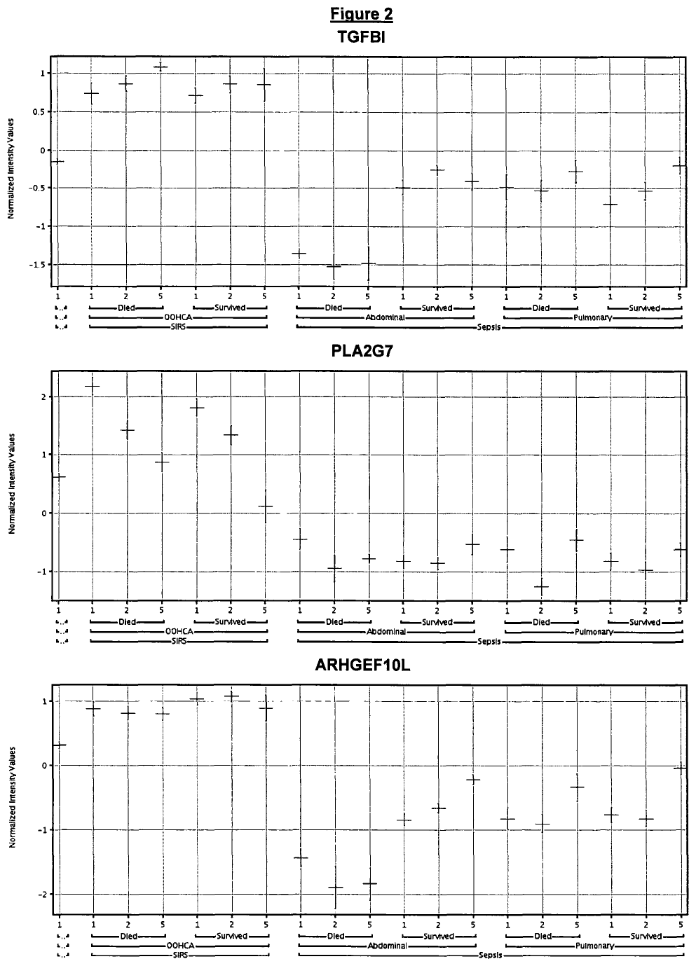

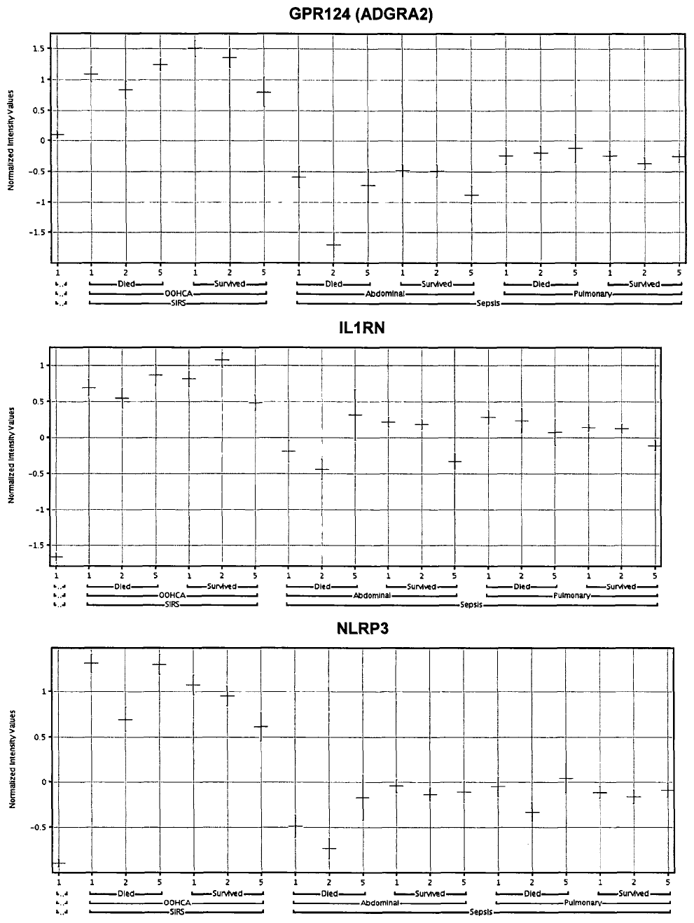

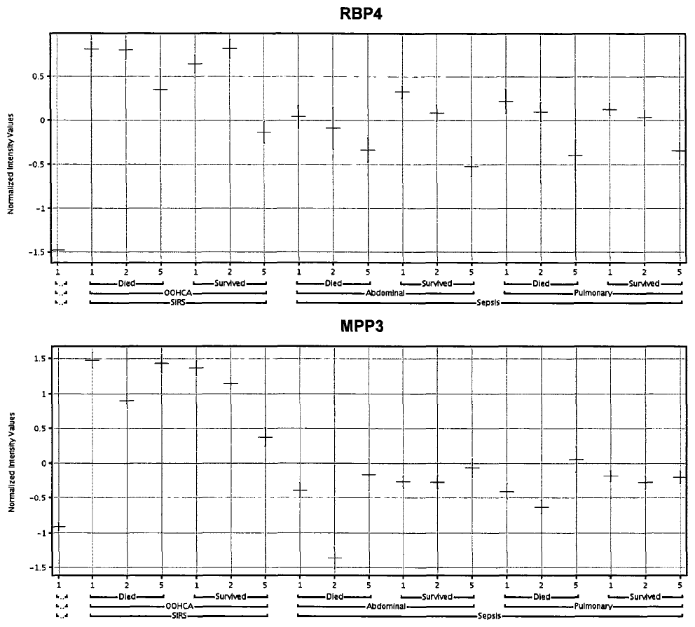

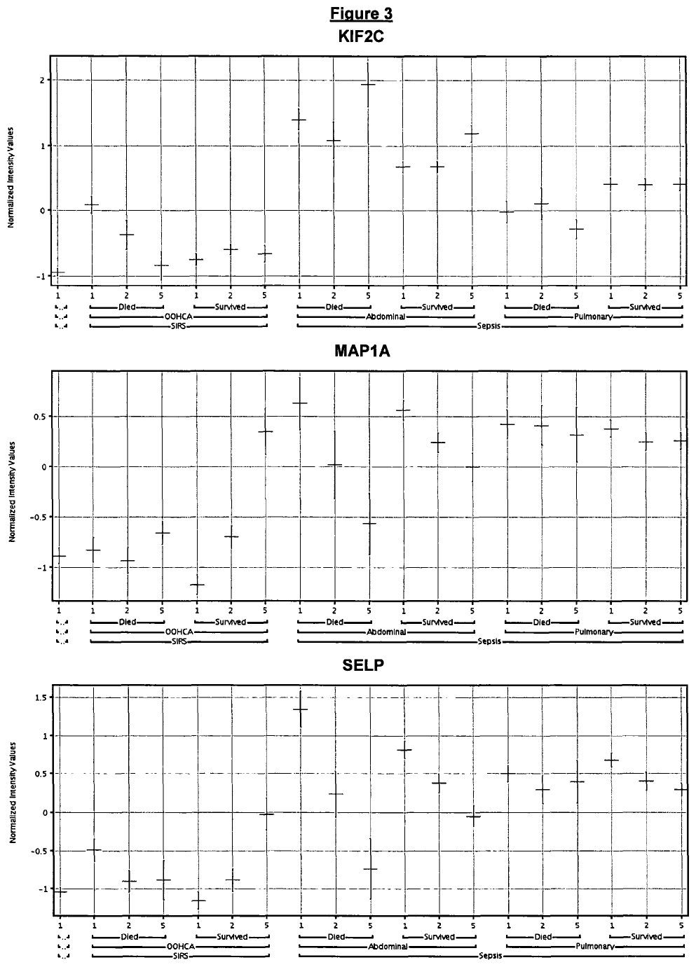

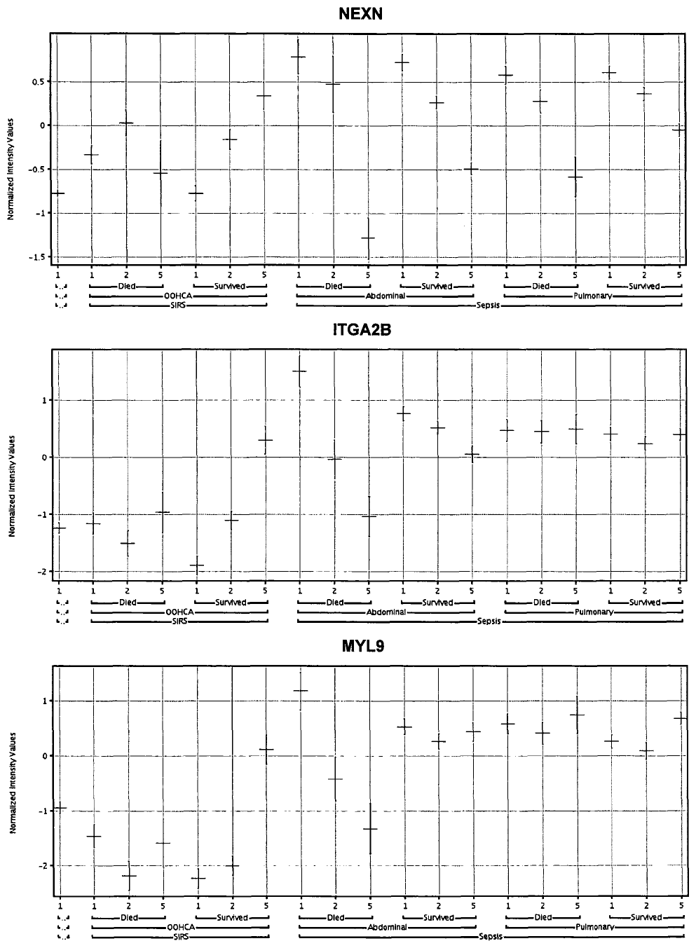

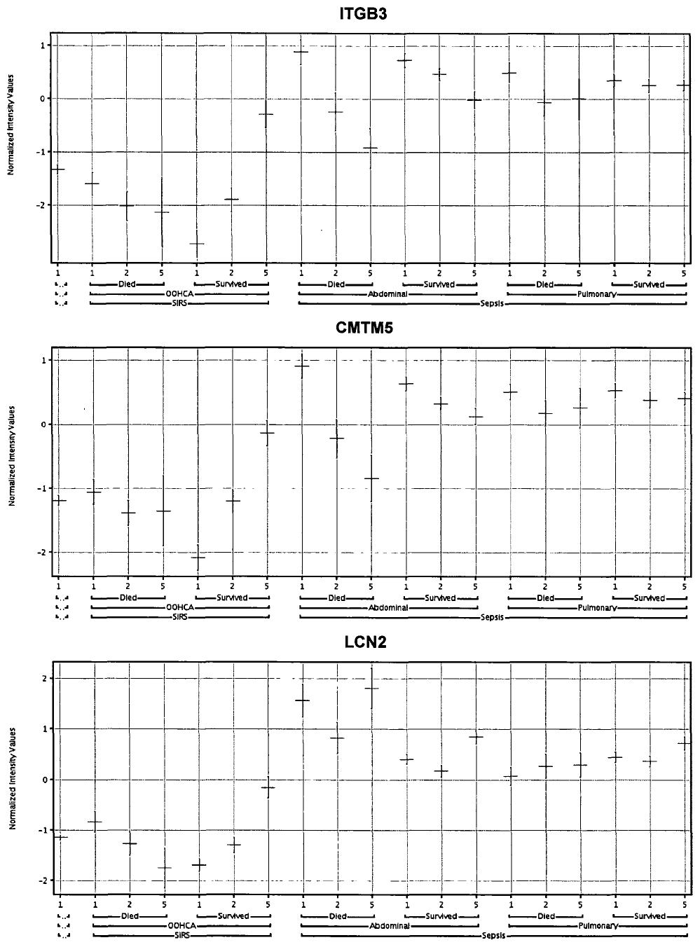

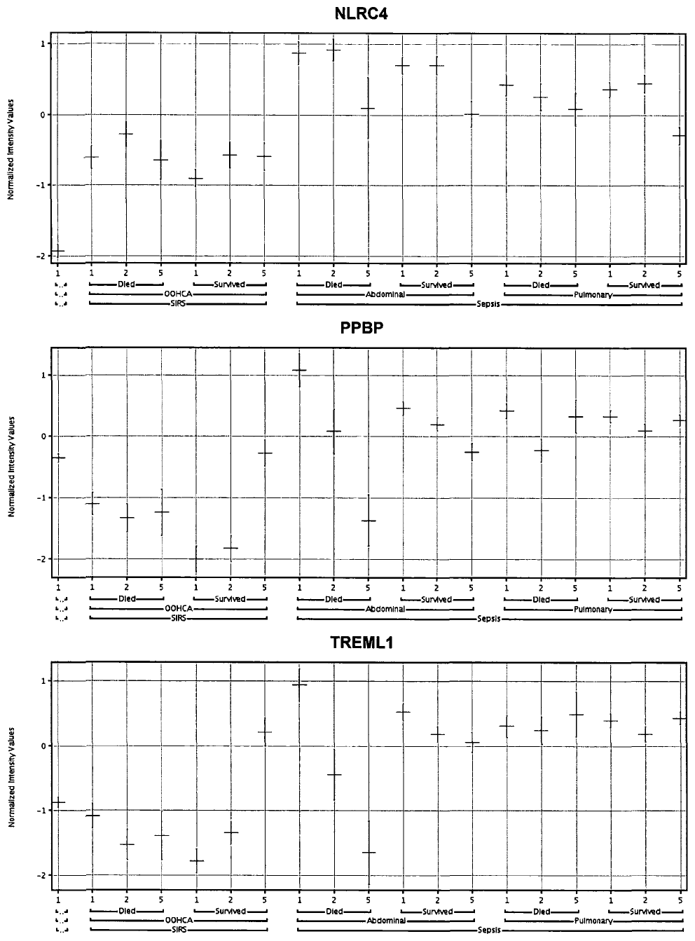

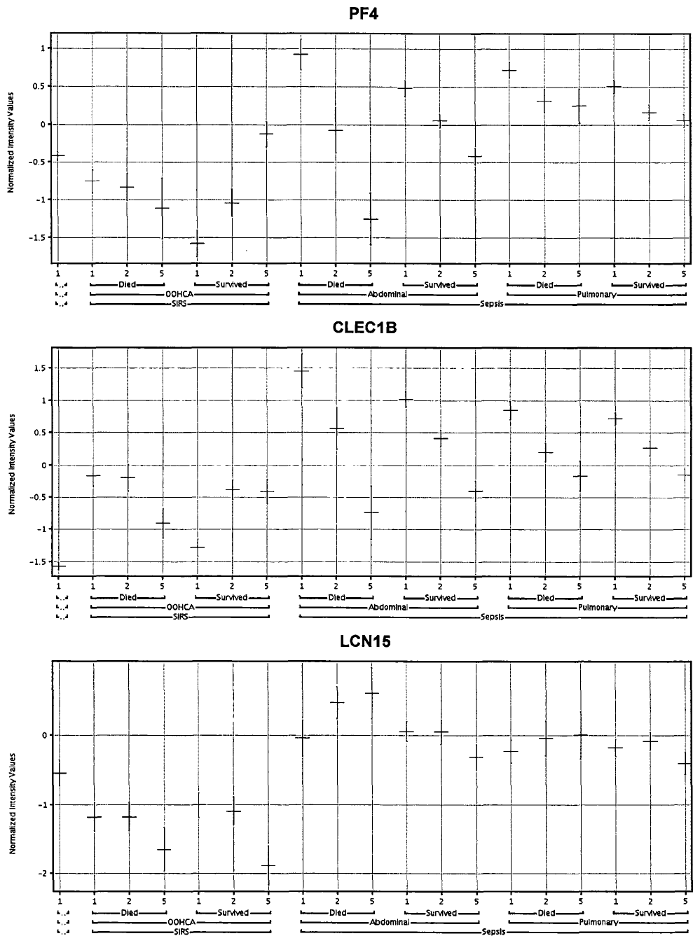

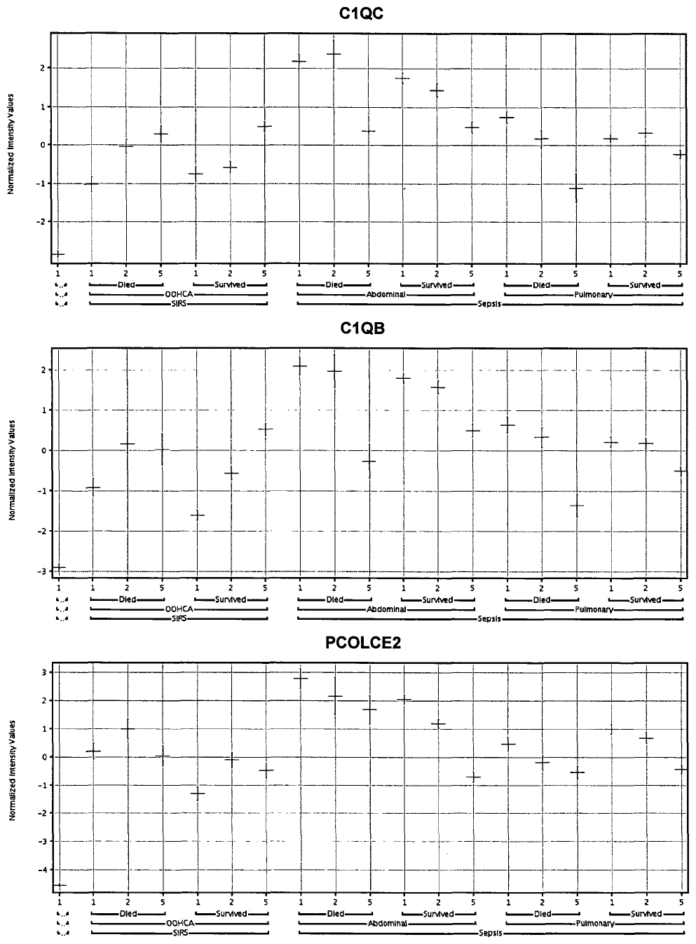

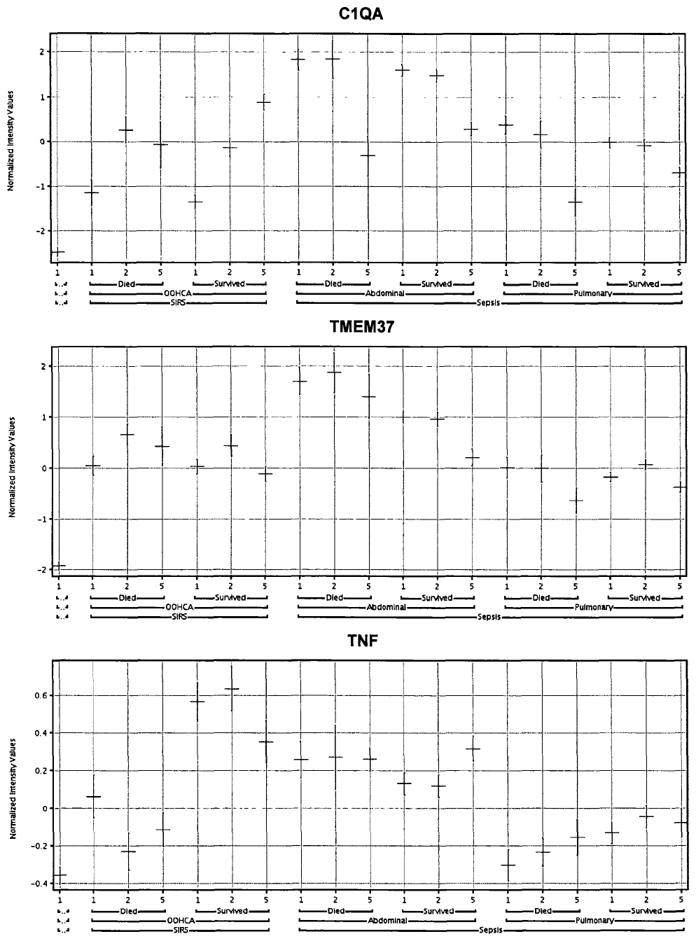

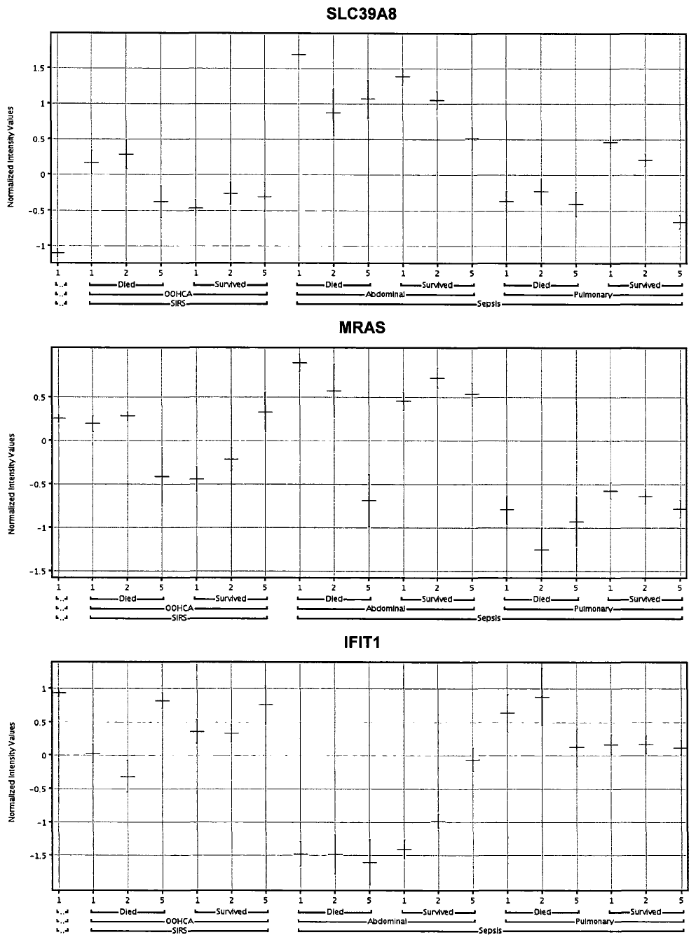

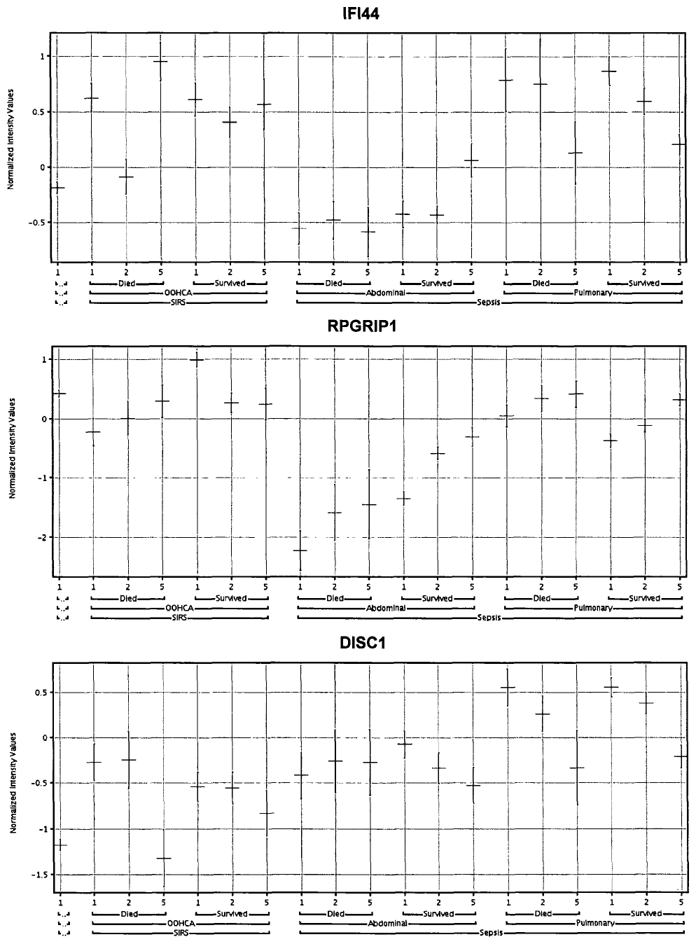

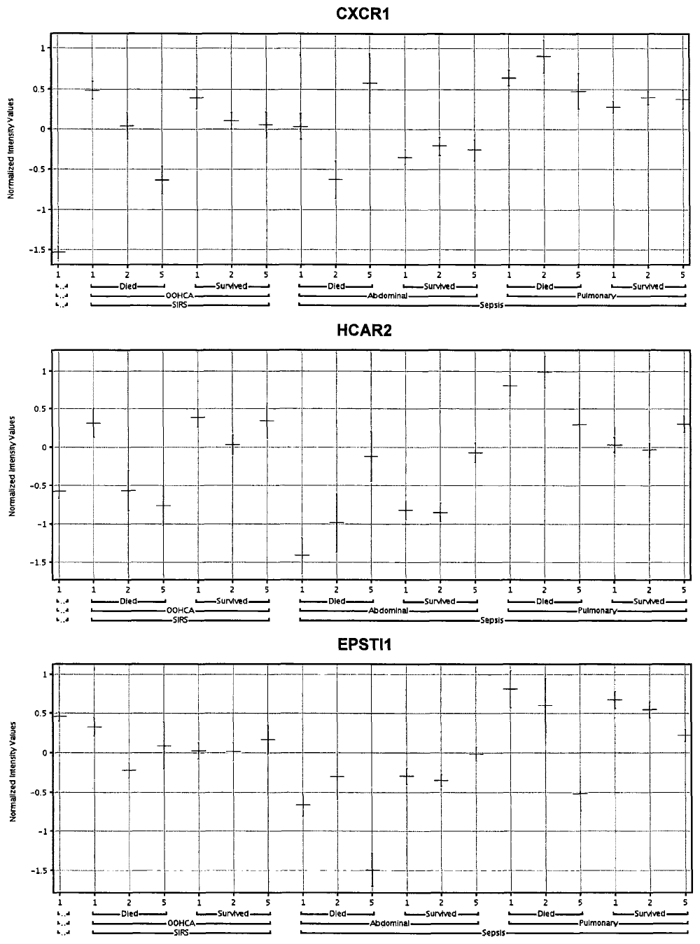

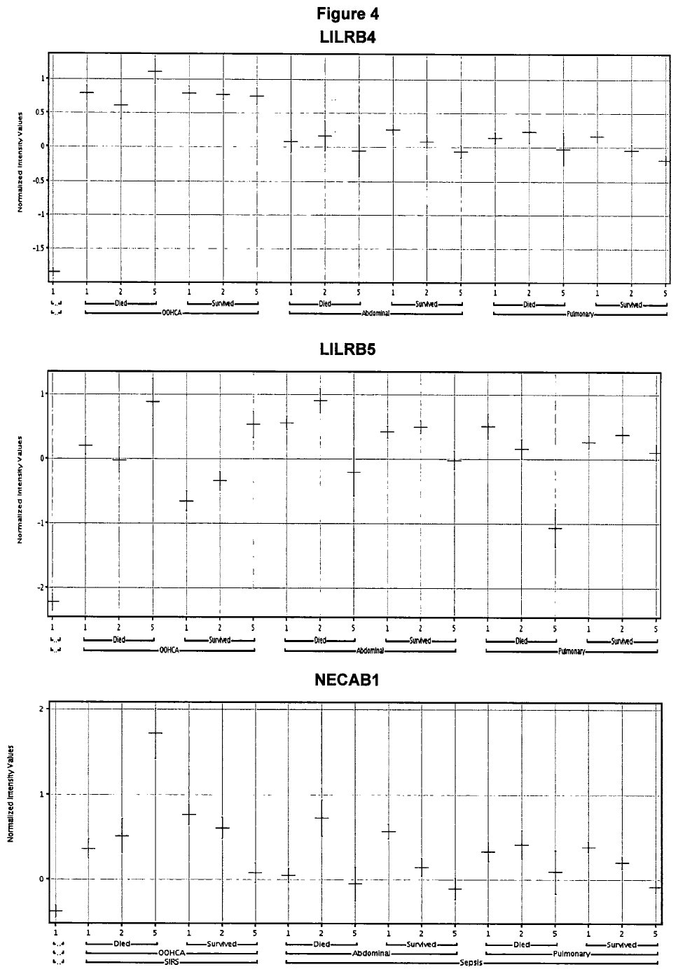

### EXAMPLE 1: IDENTIFICATION OF BIOMARKERS

### Patients:

Patients with severe sepsis and septic shock were recruited for the study based on the following criteria:

### 1. Age=>16

2. Diagnosis of severe sepsis
SEPSIS is defined as a (1) DEFINED FOCUS OF INFECTION AND (2) at least TWO systemic inflammatory response syndrome (SIRS) criteria.
   a) (1) DEFINED FOCUS OF INFECTION is indicated by either
      i. An organism grown in blood or sterile site OR
      ii. An abscess or infected tissue (e.g. pneumonia, peritonitis, urinary tract, vascular line infection, soft tissue, etc).
   b) (2) The 4 SIRS criteria are:
      i. CORE TEMPERATURE >38° C. or <36° C. (Core temperature is rectal, urinary bladder, central line, or tympanic). If oral, inguinal or axillary temperatures are used, add 0.5° C. to the measured value. Hypothermia <36° C. must be confirmed by core temperature only. Use the most deranged value recorded in the 24 hours before ICU admission.
      ii. HEART RATE >90 beats/minute. If patient had an atrial arrhythmia, record the ventricular rate. If patients have a known medical condition or are receiving treatment that would prevent tachycardia (for example, heart block or beta blockers), they must meet two of the remaining three SIRS criteria. Use the most deranged value recorded in the 24 hours before ICU admission.
      iii. RESPIRATORY RATE >20 breaths per minute or a PaCO₂<4.3 kPa (32 mmHg) or mechanical ventilation for an acute process. Use the most deranged respiratory rate or PaCO₂ recorded in the 24 hours before ICU admission.
      iv. WHITE BLOOD CELL COUNT of >12×10⁹/l or <4×10⁹/l or >10% immature neutrophils (band forms). Use the most deranged value recorded in the 24 hours before ICU admission.
SEVERE SEPSIS is defined as SEPSIS plus at least ONE ORGAN FAILURE, except when that organ failure was already present 48 hours before the onset of sepsis.
ORGAN FAILURE is defined as a Sequential Organ Failure Assessment (SOFA) score ≥2 for the organ in question
   3. Presenting to hospital with abdominal or pulmonary sepsis of less than 72 hours duration
   4. Patient already has or will require arterial cannulation as part of standard treatment

Patients with SIRS (Critically Ill patients without infection) were recruited based on the following criteria:

1. Patients admitted to the ICU following out-of hospital cardiac arrest
2. SIRS criteria as above
3. Organ failure criteria as above
4. Patients must not be receiving antibiotics for treatment of known or suspected infection
5. Patient already has or will require arterial cannulation as part of standard treatment

### Exclusion Criteria

age <16
pregnant
severe immune deficiency, for example
a diagnosis of AIDS
anti-rejection transplant drugs
methotrexate
high dose corticosteriod treatment (>10 mg prednisolone/day or equivalent)
Severe Liver Failure
Childs III or worse

Volunteers above the age of 18 were recruited for use as healthy control individuals. Exclusion criteria included:
Presence of current or chronic infection
severe immune deficiency, for example a diagnosis of AIDS
anti-rejection transplant drugs, methotrexate, high dose corticosteriod treatment (>10 mg prednisolone/day or equivalent)
severe acute or chronic liver disease
presence of malignancy which is currently treated with chemo- or radiotherapy
Irreversible disease with <6 months prognosis
   Sample Collection and Processing: Blood samples were collected from the sepsis patients (abdominal sepsis N=54 and pulmonary sepsis patients N=76) and SIRS patients (N=38) at day 1, day 2, and day 5 of admittance to an intensive care unit (ICU) and on discharge. One blood sample was collected from healthy volunteers (N=30) similar to day 1 blood sampling of recruited patients.

5 ml of whole heparinised blood obtained from patients was mixed with Erythrocyte Lysis (EL) Buffer (Qiagen) followed by incubation on ice for 10-15 minutes. Peripheral blood leukocytes (PBLs) were recovered from erythrocyte-lysed blood by centrifugation at 400×g for 10 minutes at 4° C. and re-suspended in a further 2 ml of EL buffer. PBLs were again recovered by centrifugation as described above and processed for recovery of total RNA. RNA was then prepared from patient PBLs using a semi-automated process on the Maxwell® 16 platform using the Maxwell® 16 LEV simplyRNA Blood Kit. Concentration and purity (A260/A280 ratio 1.8) were then assessed by spectrophotometry using a NanoDrop ND-1000 spectrophotometer (Thermo Scientific). Human PBL mRNA samples were labeled with Cy3 using the Agilent Quick Amp one colour labelling kit and then hybridised to Human SurePrint G3 Human Gene Expression v3 8x60K Microarrays according to the manufacturer's instructions. After hybridisation and wash steps the slides were scanned using an Agilent SureScan Dx G5761AA Microarray Scanner using default settings.

### Gene Expression Analysis:

### Parametric; Analysis of Variance and Group T-Tests

Raw data were exported and analysed using the bioinformatics software Genespring 12.5, for differential gene expression and statistical analyses. Raw data were normalized to the 75th percentile followed by baseline transformation to the mean of all samples. Data were assessed for quality, then filtered on gene expression where entities in all samples and all conditions had normalised expression values within the cut-off −10.699 to 7.037. Statistically significant features were identified using one-way ANOVA or T-test analyses across all entities, using the Benjamini-Hochberg False Discovery Rate (BH-FDR) multiple testing correction at a cut-off p<0.05. Data were further analysed and depicted graphically using the heat map, hierarchical cluster analysis and other functions in Genespring 12.5, using default settings. To identify differentially expressed entities between patients having sepsis or SIRS and healthy individuals, fold change cut-off analysis was conduced using a default cut-off setting of >2.5.

### Non Parametric; Artificial Neural Network Analyses

A stepwise Artificial Neural Network approach was used to identify an optimised gene signature panel comprising orthogonal genes from a previously established gene biomarker set for sepsis. The approach was repeated 5 times to 10 stepwise additions to assess the stability of the identified gene set given the number of cases provided. This was achieved using a stochastic data selection approach incorporating Monte Carlo cross validation.

### Architecture

The ANN modelling undertaken used a supervised learning approach applied to a three-layer multi-layer perceptron architecture. The initial weights matrix was randomised with a standard deviation of 0.1 to reduce the risk of over fitting the data. The ANN architecture was initially constrained to two hidden nodes in the hidden layer also for this reason. Hidden nodes and the output node incorporated a sigmoidal transfer function. During training weights were updated by a feed forward back propagation algorithm (Rumelhart, Hinton et al. 1986). Learning rate and momentum were set at 0.1 and 0.5 respectively. The output node was coded as 0 if the patient showed no evidence of sepsis, and 1 if sepsis was evident. Similar assessments were performed for patients with SIRS.

### Monte Carlo Cross Validation

Prior to ANN training, the data was randomly divided into three subsets; 60% for training, 20% for testing (to assess model performance during the training process) and 20% for validation (to independently test the model on data completely blind to the model). This process of random sample cross validation also contributed to the reduction of over-fitting to the data and assess how well the model would perform on a blind data set.

### Stepwise Model Development for Consistency Analysis

The normalised intensity of each gene was used as an individual input in the ANN model, creating n individual models, where n was the number of genes in the provided panel. These n models were then split into three subsets (described above) and trained. This random resampling and training process was repeated 50 times to generate predictions and associated error values for each sample with respect to the validation (blind) data. Inputs were ranked in ascending order based on predictive error and the gene that performed with the lowest error was selected for further training. Next, each of the remaining genes were sequentially added to the previous best gene, and were used in combination in a model, creating n−1 models each containing two genes as inputs. Training was repeated and performance evaluated. The model with the highest modelling performance was again selected and the process repeated creating n−2 models each containing three inputs. This process was repeated until no significant gain was evident from the addition of further inputs. This resulted in a final model containing the expression signature that most accurately classified the patients according to development of sepsis or SIRS. A set of 85 biomarkers was identified as being useful for diagnosis and monitoring of the systemic inflammatory conditions sepsis and SIRS. The biomarkers identified as summarised below in Tables 1-4.

TABLE 1
Biomarkers of systemic inflammation

| | Reference | Corrected |
|---|---|---|
| | SEQ ID NO | P value |
| Biomarkers of inflammation | | |
| ADM | 1 | 0.00000E+00 |
| CD177 | 2 | 0.00000E+00 |
| FAM20A | 3 | 0.00000E+00 |
| IL10 | 4 | 0.00000E+00 |
| METTL7B | 5 | 0.00000E+00 |
| MMP9 | 6 | 0.00000E+00 |
| RETN | 7 | 0.00000E+00 |
| TDRD9 | 8 | 0.00000E+00 |
| ITGA7 | 9 | 0.00000E+00 |
| BMX | 10 | 8.40000E−45 |
| HP | 11 | 1.62300E−42 |
| IGFBP2 | 12 | 1.06650E−40 |
| ALPL | 13 | 8.93141E−38 |
| DACH1 | 14 | 2.70941E−33 |
| IL1R1 | 15 | 9.00631E−32 |
| OLAH | 16 | 2.28395E−30 |
| IL1R2 | 17 | 3.78448E−30 |
| CYP19A1 | 18 | 7.40707E−25 |
| MMP8 | 19 | 3.19970E−23 |
| TGFA | 20 | 2.86E−17 |
| VSTM1 | 21 | 7.77E−13 |
| Biomarkers of recovery | | |
| FCER1A | 22 | 0.00000E+00 |
| KLRK1 | 23 | 5.49388E−38 |
| KLRB1 | 24 | 2.32E−36 |
| DAAM2 | 25 | 5.58531E−34 |
| HLA-DRA | 26 | 2.54E−32 |
| BCL11B | 27 | 6.59918E−30 |
| ITM2A | 28 | 1.36751E−28 |
| SLAMF6 | 29 | 7.98235E−28 |
| HLA-DPB1 | 30 and 31 | 4.19667E−27 |
| CD160 | 32 | 1.72E−22 |
| KLRF1 | 33 | 5.17E−21 |
| CD2 | 34 | 2.62E−20 |
| LGALS2 | 35 | 3.15157E−10 |
| NPPC | 36 | 2.69E−08 |
| MYCL | 37 and 38 | 1.50E−07 |
| MX1 | 39 | 3.29E−04 |
| CCL5 | 40 | 1.81E−16 |

Table 1 lists the genes identified as biomarkers of systemic inflammatory conditions using the above methods. The identified biomarkers are useful for diagnosis of systemic inflammatory conditions (e.g. see the biomarkers of inflammation shown in the top part of the table). The identified biomarkers are also useful for monitoring of systemic inflammatory conditions (e.g. see the biomarkers of inflammation shown in bottom part of the table). The final column gives the corrected ANOVA p value illustrating the significance of the biomarkers.

TABLE 2
Biomarkers of SIRS

| | Reference | Corrected |
|---|---|---|
| Biomarker | SEQ ID NO | p value |
| MYCL | 37 and 38 | 1.68E−21 |
| TGFBI | 41 | 1.34E−17 |
| PLA2G7 | 42 | 1.03E−14 |
| ARHGEF10L | 43 | 1.50E−14 |
| GPR124 | 44 | 2.25E−10 |
| ID1RN | 45 | 9.13350E−41 |
| NLRP3 | 46 | 6.43E−28 |
| RBP4 | 47 | 2.95E−21 |
| MPP3 | 48 | 7.99E−13 |

Table 2 lists the genes identified as biomarkers of SIRS using the above methods. The final column gives the corrected ANOVA p value illustrating the significance of the biomarkers.

TABLE 3
Biomarkers of sepsis

| | | Reference | |
|---|---|---|---|
| | | SEQ | Corrected |
| | Biomarker | ID NO | p value |
| Level of biomarker | | | |
| observed in sepsis patients | | | |
| High in abdominal and | KIF2C | 49 | 4.83E−26 |
| pulmonary sepsis | | | |
| High in abdominal and | MAP1A | 50 | 2.46E−18 |
| pulmonary sepsis | | | |
| High in abdominal and | SELP | 51 | 2.27E−11 |
| pulmonary sepsis | | | |
| High in abdominal and | NEXN | 52 | 2.25E−10 |
| pulmonary sepsis | | | |
| High in abdominal and | ITGA2B | 53 | 4.65E−10 |
| pulmonary sepsis | | | |
| High in abdominal and | MYL9 | 54 | 1.06E−09 |
| pulmonary sepsis | | | |
| High in abdominal and | ITGB3 | 55 | 2.61E−09 |
| pulmonary sepsis | | | |
| High in abdominal and | CMTM5 | 56 | 4.91E−09 |
| pulmonary sepsis | | | |
| High in abdominal and | LCN2 | 57 | 1.04E−08 |
| pulmonary sepsis | | | |
| High in abdominal and | NLRC4 | 58 | 3.10376E−24 |
| pulmonary sepsis | | | |
| High in abdominal and | PPBP | 59 | 2.66E−08 |
| pulmonary sepsis | | | |
| High in abdominal and | TREML1 | 60 | 2.73E−08 |
| pulmonary sepsis | | | |
| High in abdominal and | PF4 | 61 | 2.83E−08 |
| pulmonary sepsis | | | |
| High in abdominal and | CLEC1B | 62 | 2.79E−07 |
| pulmonary sepsis | | | |
| High in abdominal and | LCN15 | 63 | 3.07E−06 |
| pulmonary sepsis | | | |
| Biomarker of abdominal | | | |
| sepsis | | | |
| High in abdominal sepsis | C1QC | 64 | 0.00000E+00 |
| High in abdominal sepsis | C1QB | 65 | 8.49000E−43 |
| High in abdominal sepsis | PCOLCE2 | 66 | 5.85317E−36 |
| High in abdominal sepsis | C1QA | 67 | 2.12937E−30 |
| High in abdominal sepsis | TMEM37 | 68 | 2.30571E−24 |
| High in abdominal sepsis | TNF | 69 | 4.70E−07 |
| High in abdominal sepsis | SLC39A8 | 70 and 71 | 1.81E−05 |
| High in abdominal sepsis | MRAS | 72 | 1.27E−04 |
| Low in abdominal sepsis | IFIT1 | 73 | 1.48E−04 |
| Low in abdominal sepsis | IFI44 | 74 | 7.62E−04 |
| Low in abdominal sepsis | RPGRIP1 | 75 | 7.79E−04 |
| Biomarker of pulmonary | | | |
| sepsis | | | |
| High in pulmonary sepsis | DISC1 | 76 | 6.02116E−36 |
| High in pulmonary sepsis | CXCR1 | 77 | 1.17E−20 |
| High in pulmonary sepsis | HCAR2 | 78 | 7.62E−04 |
| High in pulmonary sepsis | EPSTI1 | 79 | 7.62E−04 |

Table 3 lists the genes identified as biomarkers of sepsis using the above methods. The table also provides an indication as to whether the gene was observed to be elevated in abdominal or pulmonary sepsis. The final column gives the corrected ANOVA p value illustrating the significance of the biomarkers.

TABLE 4
Biomarkers associated with prognosis of survival

| | Reference | Corrected |
|---|---|---|
| Biomarker | SEQ ID NO. | p value |
| LILRB4 | 80 | 2.03E−32 |
| LILRB5 | 81 | 2.47E−22 |
| NECAB1 | 82 | 1.28E−07 |
| NECAB2 | 83 | 2.06E−05 |
| PKHD1 | 84 | 4.96E−05 |
| PKD1 | 85 | 3.15E−03 |

Table 4 lists the genes identified as biomarkers of patient survival using the above methods. The final column gives the corrected ANOVA p value illustrating the significance of the biomarkers.

TABLE 5

| | | | | | | | |
|---|---|---|---|---|---|---|---|
| | Day 1 | Day 2 | Day 5 | Day 1 | Day 2 | Day 5 | |
| Gene | (Died) | (Died) | (Died) | (Survived) | (Survived) | (Survived) | Discharged |

Fold change data observed for the patients having non−infective SIRS:

| | | | | | | | |
|---|---|---|---|---|---|---|---|
| ADM | 12.76 | 11.22 | 5.70 | 11.68 | 14.20 | 11.25 | 12.33 |
| ALPL | 10.74 | 8.46 | 3.78 | 7.85 | 8.44 | 6.35 | 4.76 |
| ARHGEF10L | 1.48 | 1.42 | 1.41 | 1.65 | 1.69 | 1.50 | 1.88 |
| A33_P3799936 | | | | | | | |
| ARHGEF10L | −1.06 | −1.07 | −2.08 | −1.01 | 1.01 | 1.02 | −1.10 |
| A33_P3215575 | | | | | | | |
| BMX | 48.54 | 28.97 | 10.34 | 27.47 | 28.18 | 9.83 | 10.53 |
| C1QA | 2.50 | 6.64 | 5.26 | 2.18 | 5.00 | 10.21 | 6.11 |
| C1QB | 3.98 | 8.49 | 7.67 | 2.46 | 5.10 | 10.80 | 5.74 |
| C1QC | 3.52 | 7.01 | 8.80 | 4.25 | 4.84 | 10.09 | 5.01 |
| CD177 | 84.52 | 45.72 | 6.97 | 29.94 | 53.15 | 15.96 | 7.28 |
| A23 P0011751 | | | | | | | |
| CD177 | 96.09 | 55.1 | 10.4 | 39.8 | 65.02 | 21.29 | 11.51 |
| A23 P259863 | | | | | | | |
| CCL5 | 3.036 | 3.61 | 10.49 | 5.49 | 4.02 | 2.55 | −3.126 |
| CLEC1B | 2.64 | 2.59 | 1.59 | 1.22 | 2.27 | 2.24 | 1.15 |
| CMTM5 | 1.09 | −1.15 | −1.12 | −1.86 | −1.01 | 2.07 | 1.56 |
| CXCR1 | 4.04 | 2.97 | 1.87 | 3.78 | 3.10 | 3.00 | 2.14 |
| CYP19A1 | 7.60 | 8.37 | 3.88 | 4.16 | 4.05 | 2.59 | 1.36 |
| CYP19A1 | 1.52 | 1.98 | 3.17 | 2.37 | 1.99 | 1.71 | 1.13 |
| DAAM2 | 9.27 | 7.60 | 9.25 | 6.51 | 4.62 | 5.76 | 2.50 |
| DAAM2 | 18.08 | 12.27 | 10.35 | 7.67 | 5.48 | 8.72 | 3.37 |
| DACH1 | 8.03 | 5.96 | 13.08 | 4.81 | 5.44 | 5.30 | 6.75 |
| DACH1 | 6.42 | 4.76 | 7.41 | 3.93 | 4.11 | 4.00 | 3.95 |
| DISC1 | 1.87 | 1.90 | −1.11 | 1.55 | 1.54 | 1.27 | −1.26 |
| A21 P000047 | | | | | | | |
| DISC1 | −1.02 | −1.09 | −2.09 | −1.38 | −.135 | −1.31 | −2.43 |
| A24 P83787 | | | | | | | |
| DISC1 | −1.08 | 1.08 | −1.29 | 1.15 | −1.03 | −.122 | −2.45 |
| A21 P0000050 | | | | | | | |
| EPSTI1 | −1.09 | −1.60 | −1.29 | −1.35 | −1.36 | −1.23 | −1.29 |
| FAM20A | 66.73 | 61.83 | 59.59 | 43.80 | 59.31 | 42.12 | 26.28 |
| A32 P108254 | | | | | | | |
| FAM20A | 32.21 | 26.19 | 13.07 | 17.38 | 22.73 | 21.01 | 14.17 |
| A23 P352952 | | | | | | | |
| GPR124 | 1.99 | 1.67 | 2.22 | 2.68 | 2.40 | 1.63 | 2.05 |
| HCAR2 | 1.86 | 1.01 | −1.14 | 1.95 | 1.53 | 1.90 | 2.05 |
| HP | 16.48 | 21.18 | 27.79 | 16.57 | 23.68 | 13.80 | 17.43 |
| IFI44 | 1.76 | 1.08 | 2.21 | 1.74 | 1.51 | 1.69 | 2.23 |
| IFIT1 | −1.87 | −2.38 | −1.08 | −1.49 | −1.52 | −1.13 | 1.24 |
| IGFBP2 | 7.08 | 8.52 | 25.16 | 5.34 | 11.38 | 15.11 | 8.53 |
| IL10 | 9.16 | 6.64 | 7.86 | 5.38 | 5.76 | 4.78 | 4.96 |
| IL1R1 | 7.99 | 6.96 | 5.49 | 4.09 | 5.68 | 6.81 | 6.16 |
| IL1R1 | 5.93 | 5.88 | 3.41 | 3.55 | 3.69 | 4.60 | 3.24 |
| IL1R2 | 16.73 | 9.91 | 4.95 | 3.65 | 4.08 | 5.37 | 2.93 |
| IL1RN | 5.09 | 4.60 | 5.75 | 5.53 | 6.63 | 4.39 | 5.33 |
| ITGA2B | 1.05 | −1.21 | 1.21 | −1.57 | 1.09 | 2.89 | 1.56 |
| ITGA7 | 10.23 | 11.84 | 16.50 | 5.20 | 8.16 | 9.65 | 8.53 |
| ITGB3 | −1.20 | −1.61 | −1.74 | −2.62 | −1.49 | 2.06 | −1.14 |
| KIF2C | 2.04 | 1.48 | 1.07 | 1.14 | 1.27 | 1.21 | 1.09 |
| LCN15 | −1.55 | −1.55 | −2.15 | −1.38 | −1.46 | −2.53 | −2.09 |
| LCN2 | 1.24 | −1.09 | −1.53 | −1.46 | −1.11 | 1.99 | −1.04 |
| LGALS2 | −3.29 | −2.05 | 1.56 | −2.01 | −1.77 | −2.26 | −1.37 |
| MAP1A | 1.04 | −1.03 | 1.17 | −1.22 | 1.14 | 2.35 | 1.11 |
| METTL7B | 19.59 | 21.29 | 27.43 | 11.40 | 15.60 | 10.67 | 11.96 |
| MMP8 | 6.73 | 5.34 | 4.62 | 2.64 | 2.68 | 3.54 | 2.29 |
| MMP9 | 64.93 | 40.17 | 17.27 | 66.89 | 53.46 | 17.91 | 15.55 |
| MPP3 | 5.25 | 3.52 | 5.07 | 4.85 | 4.16 | 2.44 | 2.97 |
| MRAS | −1.04 | 1.02 | −1.59 | −1.62 | −1.38 | 1.05 | −1.05 |
| MYCL | −1.12 | 1.25 | 2.11 | 1.37 | 1.40 | 1.57 | 2.05 |
| MYL9 | −1.43 | −2.36 | −1.56 | −2.46 | −2.09 | 2.08 | −1.19 |
| NEXN | 1.36 | 1.74 | 1.17 | −1.00 | 1.53 | 2.16 | 1.25 |
| NLRC4 | 2.50 | 3.14 | 2.44 | 2.04 | 2.57 | 2.53 | 1.42 |
| NLRP3 | 4.58 | 2.99 | 4.54 | 3.90 | 3.57 | 2.83 | 3.42 |
| OLAH | 14.67 | 11.01 | 21.25 | 4.87 | 5.10 | 11.28 | 6.73 |
| PCOLCE2 | 26.63 | 46.09 | 23.79 | 9.34 | 21.46 | 16.50 | 23.87 |
| PF4 | −1.26 | −1.33 | −1.62 | −2.23 | −1.54 | 1.22 | −1.64 |
| PLA2G7 | 2.96 | 1.76 | 1.19 | 2.29 | 1.64 | −1.41 | 1.41 |
| PPBP | −1.69 | −1.99 | −1.86 | −3.14 | −2.80 | 1.06 | −1.51 |
| RBP4 | 4.88 | 4.87 | 3.55 | 4.35 | 4.90 | 2.54 | 2.27 |
| RETN | 31.59 | 22.53 | 8.65 | 24.30 | 25.93 | 4.95 | 5.13 |
| RPGRIP1 | −1.57 | −1.33 | −1.10 | 1.48 | −1.12 | −1.14 | −1.00 |
| SELP | 1.46 | 1.10 | 1.12 | −1.08 | 1.11 | 2.02 | 1.73 |
| SLAMF6 | −9.03 | −6.76 | −10.21 | −8.49 | −11.62 | −6.07 | −8.10 |
| SLC39A8 | 2.42 | 2.61 | 1.65 | 1.55 | 1.79 | 1.74 | 1.85 |
| SLC39A8 | 3.17 | 2.99 | 2.48 | 1.45 | 2.49 | 1.91 | 2.31 |
| TDRD9 | 9.07 | 13.78 | 12.47 | 7.01 | 12.75 | 10.53 | 10.55 |
| TGFA | 4.02 | 3.80 | 4.23 | 4.05 | 4.24 | 2.20 | 3.07 |
| TGFBI | 1.84 | 2.01 | 2.35 | 1.82 | 2.01 | 2.00 | 2.42 |
| TMEM37 | 3.92 | 6.03 | 5.10 | 3.88 | 5.16 | 3.52 | 3.08 |
| TNF | 1.34 | 1.09 | 1.18 | 1.89 | 1.99 | 1.64 | 1.63 |
| TREML1 | −1.16 | −1.58 | −1.43 | −1.88 | −1.39 | 2.12 | 1.24 |
| VSTM1 | 2.95 | 3.20 | 4.38 | 3.46 | 4.89 | 3.52 | 3.37 |
| LILRB4 | 6.22 | 5.48 | 7.70 | 6.22 | 6.13 | 6.01 | 3.25 |
| LILRB5 | 5.39 | 4.65 | 8.63 | 2.99 | 3.71 | 6.79 | 5.04 |
| NECAB1 | 1.67 | 1.86 | 4.28 | 2.21 | 1.98 | 1.37 | 1.14 |
| NECAB2 | 1.64 | 2.07 | 2.48 | 2.44 | 1.82 | 3.26 | 1.99 |
| PKD1 | 1.06 | 1.12 | 1.83 | 1.51 | 1.43 | 1.43 | 1.28 |
| A21 P0011417 | | | | | | | |
| PKD1 | 1.19 | 1.26 | 1.65 | 1.61 | 1.51 | 1.21 | 1.29 |
| A21 P0011418 | | | | | | | |
| PKD1 | 1.13 | 1.29 | 1.99 | 1.32 | 1.25 | 1.47 | 1.47 |
| A21 P0011419 | | | | | | | |
| PKHD1 | 1.64 | 1.80 | 5.37 | 2.31 | 2.07 | 1.43 | 1.13 |
| A23 P402187 | | | | | | | |
| PKHD1 | 1.80 | 1.88 | 4.10 | 2.39 | 2.13 | 1.54 | 1.13 |
| A33 P3387420 | | | | | | | |
| PKHD1 | 1.62 | 1.92 | 3.44 | 2.45 | 2.14 | 1.67 | 1.43 |
| A23 P424617 | | | | | | | |
| BCL11B | −5.40 | −4.33 | −4.87 | −4.32 | −4.08 | −3.45 | −2.58 |
| CD160 | −8.64 | −5.98 | −5.31 | −4.81 | −3.85 | −2.93 | −1.98 |
| CD2 | −2.52 | −1.90 | −1.52 | −2.07 | −1.83 | −1.87 | −1.52 |
| FCER1A | −4.43 | −4.68 | −18.52 | −3.23 | −5.88 | −6.01 | −4.05 |
| HLA−DPB1 | −1.48 | −1.40 | −2.63 | −1.63 | −1.89 | −1.69 | −1.46 |
| HLA−DRA | −2.36 | −2.51 | −4.49 | −1.83 | −2.43 | −2.23 | −2.13 |
| ITM2A | −7.47 | −5.46 | −8.00 | −8.28 | −6.37 | −4.09 | −4.10 |
| KLRB1 | −4.25 | −4.15 | −4.68 | −3.78 | −4.37 | −3.51 | −2.22 |
| KLRF1 | −3.91 | −2.08 | −4.00 | −3.82 | −4.33 | −3.20 | −1.80 |
| KLRK1 | −10.31 | −9.22 | −12.30 | −11.26 | −10.70 | −7.17 | −4.84 |
| MX1 | −1.05 | −1.39 | −1.17 | −1.10 | −1.13 | 1.06 | 1.18 |
| NPPC | 1.70 | 2.49 | 5.21 | 2.88 | 2.54 | 2.10 | 1.71 |

Fold change data observed for the patients having abdominal sepsis:

| | | | | | | | |
|---|---|---|---|---|---|---|---|
| ADM | 18.02 | 13.38 | 11.65 | 14.42 | 12.70 | 9.65 | 8.40 |
| ALPL | 12.45 | 9.17 | 14.34 | 9.03 | 9.54 | 6.96 | 4.78 |
| ARHGEF10L | −3.35 | −4.60 | −4.43 | −2.22 | −1.96 | −1.44 | −1.19 |
| A33_P3799936 | | | | | | | |
| ARHGEF10L | −4.47 | −4.65 | −6.87 | −2.81 | −2.52 | −1.94 | −1.82 |
| A33_P3215575 | | | | | | | |
| BMX | 65.49 | 33.30 | 57.75 | 25.57 | 19.83 | 17.75 | 15.63 |
| C1QA | 19.63 | 19.88 | 4.44 | 16.80 | 15.48 | 6.77 | 6.57 |
| C1QB | 32.12 | 29.46 | 6.26 | 26.10 | 22.45 | 10.62 | 8.01 |
| C1QC | 32.73 | 37.30 | 9.33 | 24.37 | 19.57 | 9.96 | 7.33 |
| CD177 | 361.83 | 181.90 | 166.05 | 156.28 | 123.04 | 86.65 | 37.29 |
| A23 P0011751 | | | | | | | |
| CD177 | 399.9 | 194.2 | 192.4 | 164.5 | 123.5 | 82.9 | 45.59 |
| A23 P259863 | | | | | | | |
| CCL5 | −2.27 | −4.01 | −9.35 | −2.89 | −3.35 | −2.65 | −1.92 |
| CLEC1B | 8.18 | 4.38 | 1.78 | 6.03 | 3.96 | 2.25 | 2.68 |
| CMTM5 | 4.26 | 1.95 | 1.27 | 3.53 | 2.85 | 2.49 | 2.75 |
| CXCR1 | 2.96 | 1.87 | 4.30 | 2.27 | 2.49 | 2.42 | 2.11 |
| CYP19A1 | 11.69 | 11.27 | 15.25 | 10.40 | 7.53 | 2.04 | 3.10 |
| CYP19A1 | 2.58 | 3.42 | 2.41 | 2.94 | 1.85 | 1.31 | 1.76 |
| DAAM2 | 35.26 | 43.64 | 29.68 | 21.44 | 12.90 | 8.89 | 3.68 |
| DAAM2 | 104.78 | 171.49 | 64.55 | 32.09 | 24.21 | 14.61 | 6.94 |
| DACH1 | 6.08 | 4.84 | 11.87 | 6.02 | 5.30 | 4.62 | 4.12 |
| DACH1 | 4.11 | 3.86 | 8.56 | 4.95 | 4.42 | 3.47 | 3.51 |
| DISC1 | 1.69 | 1.88 | 1.87 | 2.14 | 1.79 | 1.57 | 1.13 |
| A21 P000047 | | | | | | | |
| DISC1 | 1.07 | 1.55 | −1.51 | 1.42 | 1.45 | −1.03 | −1.43 |
| A24 P83787 | | | | | | | |
| DISC1 | 1.18 | 1.44 | −1.45 | 1.40 | 1.41 | 1.03 | −1.27 |
| A21 P0000050 | | | | | | | |
| EPSTI1 | −2.17 | −1.70 | −3.86 | −1.69 | −1.75 | −1.39 | −1.19 |
| FAM20A | 68.88 | 86.05 | 49.80 | 73.95 | 65.01 | 29.78 | 32.82 |
| A32 P108254 | | | | | | | |
| FAM20A | 38.56 | 44.85 | 24.98 | 35.33 | 33.21 | 14.38 | 18.22 |
| A23 P352952 | | | | | | | |
| GPR124 | −1.62 | −3.48 | −1.77 | −1.49 | −1.50 | −1.97 | −1.42 |
| HCAR2 | −1.79 | −1.33 | 1.37 | −1.19 | −1.21 | 1.42 | 1.21 |
| HP | 22.89 | 21.71 | 43.00 | 22.11 | 21.79 | 11.06 | 16.20 |
| IFI44 | −1.29 | −1.22 | −1.32 | −1.18 | −1.18 | 1.19 | 1.41 |
| IFIT1 | −5.32 | −5.32 | −5.79 | −5.06 | −3.78 | −1.99 | −1.41 |
| IGFBP2 | 24.98 | 18.83 | 25.46 | 22.55 | 16.87 | 11.52 | 23.68 |
| IL10 | 15.63 | 15.18 | 5.32 | 7.14 | 6.30 | 3.73 | 3.89 |
| IL1R1 | 11.03 | 9.21 | 6.68 | 9.77 | 9.20 | 5.35 | 4.03 |
| IL1R1 | 9.38 | 7.82 | 6.12 | 10.02 | 8.18 | 5.49 | 3.19 |
| IL1R2 | 43.67 | 45.18 | 21.05 | 21.68 | 13.36 | 9.86 | 4.29 |
| IL1RN | 2.76 | 2.32 | 3.92 | 3.65 | 3.58 | 2.50 | 3.21 |
| ITGA2B | 6.67 | 2.29 | 1.15 | 3.99 | 3.37 | 2.44 | 3.24 |
| ITGA7 | 37.95 | 33.99 | 33.50 | 29.68 | 30.12 | 16.43 | 12.50 |
| ITGB3 | 4.65 | 2.13 | 1.33 | 4.18 | 3.48 | 2.49 | 3.00 |
| KIF2C | 5.11 | 4.09 | 7.40 | 3.07 | 3.07 | 4.40 | 2.67 |
| LCN15 | 1.42 | 2.03 | 2.23 | 1.52 | 1.51 | 1.17 | 1.40 |
| LCN2 | 6.56 | 3.92 | 7.78 | 2.95 | 2.49 | 3.98 | 2.76 |
| LGALS2 | −33.71 | −26.99 | −15.62 | −8.21 | −5.38 | −2.36 | −1.30 |
| MAP1A | 2.85 | 1.87 | 1.25 | 2.72 | 2.18 | 1.84 | 2.60 |
| METTL7B | 41.83 | 22.91 | 32.65 | 36.49 | 34.87 | 11.98 | 19.14 |
| MMP8 | 38.57 | 14.21 | 40.07 | 8.02 | 5.77 | 15.76 | 5.60 |
| MMP9 | 69.09 | 36.91 | 103.28 | 48.51 | 33.60 | 30.38 | 19.48 |
| MPP3 | 1.44 | −1.37 | 1.69 | 1.57 | 1.56 | 1.80 | 1.78 |
| MRAS | 1.56 | 1.25 | −1.92 | 1.15 | 1.38 | 1.21 | 1.20 |
| MYCL | −4.28 | −4.96 | −2.51 | −2.39 | −1.72 | −1.61 | −1.06 |
| MYL9 | 4.36 | 1.44 | −1.31 | 2.78 | 2.31 | 2.60 | 3.36 |
| NEXN | 2.94 | 2.37 | −1.42 | 2.81 | 2.06 | 1.22 | 1.88 |
| NLRC4 | 7.00 | 7.19 | 4.08 | 6.21 | 6.20 | 3.86 | 2.72 |
| NLRP3 | 1.33 | 1.12 | 1.65 | 1.80 | 1.69 | 1.72 | 2.15 |
| OLAH | 111.81 | 79.31 | 69.24 | 35.20 | 21.17 | 10.08 | 4.35 |
| PCOLCE2 | 158.63 | 103.55 | 74.11 | 95.70 | 53.00 | 14.20 | 9.46 |
| PF4 | 2.53 | 1.27 | −1.78 | 1.86 | 1.39 | −1.00 | 1.41 |
| PLA2G7 | −2.10 | −2.97 | −2.64 | −2.72 | −2.79 | −2.22 | −1.55 |
| PPBP | 2.70 | 1.35 | −2.03 | 1.76 | 1.47 | 1.07 | 1.37 |
| RBP4 | 2.87 | 2.63 | 2.21 | 3.50 | 2.96 | 1.94 | 2.39 |
| RETN | 43.34 | 33.27 | 34.66 | 32.56 | 20.20 | 11.74 | 9.82 |
| RPGRIP1 | −6.29 | −4.04 | −3.66 | −3.43 | −2.01 | −1.67 | 1.12 |
| SELP | 5.22 | 2.43 | 1.24 | 3.63 | 2.67 | 1.99 | 2.38 |
| SLAMF6 | −3.42 | −4.23 | −5.00 | −6.26 | −4.98 | −4.31 | −4.34 |
| SLC39A8 | 6.95 | 3.94 | 4.50 | 5.60 | 4.46 | 3.06 | 2.11 |
| SLC39A8 | 5.54 | 4.64 | 3.69 | 4.63 | 3.77 | 2.31 | 2.28 |
| TDRD9 | 30.63 | 21.31 | 23.78 | 21.71 | 19.72 | 12.25 | 9.00 |
| TGFA | 3.42 | 3.57 | 2.13 | 3.67 | 3.08 | 1.99 | 2.35 |
| TGFBI | −2.31 | −2.60 | −2.53 | −1.27 | −1.08 | −1.20 | 1.17 |
| TMEM37 | 12.43 | 13.91 | 10.08 | 7.61 | 7.42 | 4.39 | 3.53 |
| TNF | 1.53 | 1.54 | 1.53 | 1.40 | 1.39 | 1.59 | 1.64 |
| TREML1 | 3.51 | 1.34 | −1.71 | 2.63 | 2.07 | 1.90 | 2.14 |
| VSTM1 | 3.04 | 2.25 | 3.96 | 3.17 | 3.40 | 2.67 | 3.03 |
| LILRB4 | 3.80 | 4.02 | 3.46 | 4.28 | 3.81 | 3.42 | 4.00 |
| LILRB5 | 6.91 | 8.77 | 4.08 | 6.27 | 6.64 | 4.65 | 3.23 |
| NECAB1 | 1.34 | 2.15 | 1.25 | 1.93 | 1.44 | 1.21 | 1.57 |
| NECAB2 | 1.42 | 3.72 | 3.72 | 1.46 | 1.95 | 2.54 | 2.10 |
| PKD1 | −1.35 | 1.03 | 2.29 | 1.00 | 1.15 | 1.22 | 1.10 |
| A21 P0011417 | | | | | | | |
| PKD1 | −1.16 | −1.05 | 1.60 | 1.11 | −1.07 | 1.07 | 1.09 |
| A21 P0011418 | | | | | | | |
| PKD1 | −1.16 | 1.13 | 2.46 | 1.07 | 1.11 | 1.49 | 1.12 |
| A21 P0011419 | | | | | | | |
| PKHD1 | 1.41 | 2.30 | 1.31 | 1.90 | 1.50 | 1.34 | 1.60 |
| A23 P402187 | | | | | | | |
| PKHD1 | 1.52 | 2.22 | 1.25 | 2.05 | 1.42 | 1.24 | 1.48 |
| A33 P3387420 | | | | | | | |
| PKHD1 | 1.48 | 2.34 | 1.36 | 1.97 | 1.58 | 1.28 | 1.66 |
| A23 P424617 | | | | | | | |
| BCL11B | −4.74 | −4.80 | −4.44 | −6.01 | −4.67 | −3.64 | −2.68 |
| CD160 | −9.36 | −7.69 | −10.27 | −6.30 | −5.44 | −3.73 | −2.12 |
| CD2 | −3.04 | −2.89 | −2.54 | −2.77 | −2.51 | −1.95 | −1.54 |
| FCER1A | −33.69 | −36.09 | −20.29 | −26.26 | −19.06 | −12.11 | −6.01 |
| HLA−DPB1 | −3.58 | −5.27 | −6.89 | −2.79 | −2.36 | −2.46 | −1.73 |
| HLA−DRA | −3.54 | −4.95 | −6.57 | −4.49 | −3.39 | −3.24 | −2.17 |
| ITM2A | −3.57 | −4.43 | −5.24 | −4.83 | −3.79 | −3.24 | −2.81 |
| KLRB1 | −4.85 | −5.79 | −6.96 | −5.37 | −5.08 | −3.82 | −2.66 |
| KLRF1 | −3.12 | −3.86 | −8.47 | −4.83 | −4.73 | −3.74 | −2.95 |
| KLRK1 | −6.45 | −6.70 | −18.02 | −9.16 | −8.49 | −7.36 | −5.40 |
| MX1 | −2.69 | −2.51 | −2.75 | −2.20 | −2.02 | −1.36 | −1.20 |
| NPPC | 1.95 | 2.68 | 3.06 | 2.22 | 2.30 | 1.49 | 1.79 |

Fold change data observed for patients having pulmonary sepsis:

| | | | | | | | |
|---|---|---|---|---|---|---|---|
| ADM | 12.80 | 11.51 | 9.29 | 11.42 | 11.36 | 7.53 | 5.45 |
| ALPL | 11.08 | 10.71 | 7.00 | 9.95 | 9.44 | 6.55 | 4.14 |
| ARHGEF10L | | | | | | | |
| A33_P3799936 | −2.19 | −2.31 | −1.57 | −2.11 | −2.19 | −1.28 | 1.31 |
| ARHGEF10L | | | | | | | |
| A33_P3215575 | −2.96 | −3.33 | −2.35 | −2.48 | −2.75 | −1.70 | −1.05 |
| BMX | 29.56 | 25.38 | 15.36 | 21.20 | 16.64 | 12.04 | 6.00 |
| C1QA | 7.23 | 6.19 | 2.16 | 5.44 | 5.22 | 3.44 | 3.09 |
| C1QB | 11.75 | 9.49 | 2.94 | 8.77 | 8.51 | 5.29 | 3.80 |
| C1QC | 12.02 | 8.11 | 3.30 | 8.09 | 9.05 | 6.14 | 4.59 |
| CD177 | 79.88 | 41.82 | 39.98 | 104.30 | 77.61 | 43.90 | 5.89 |
| A23 P0011751 | | | | | | | |
| CD177 | 79.44 | 45.32 | 35.42 | 110.3 | 81.38 | 49.59 | 7.27 |
| A23 P259863 | | | | | | | |
| CCL5 | −2.49 | −2.24 | −1.70 | −2.40 | −2.59 | −2.12 | −1.55 |
| CLEC1B | 5.36 | 3.41 | 2.63 | 4.91 | 3.57 | 2.69 | 1.71 |
| CMTM5 | 3.23 | 2.56 | 2.72 | 3.28 | 2.97 | 3.02 | 1.95 |
| CXCR1 | 4.50 | 5.40 | 4.01 | 3.50 | 3.79 | 3.74 | 2.11 |
| CYP19A1 | 8.14 | 5.51 | 3.89 | 6.34 | 5.62 | 2.49 | 1.42 |
| CYP19A1 | 2.04 | 2.13 | 1.07 | 2.27 | 1.89 | 1.43 | 1.33 |
| DAAM2 | 22.74 | 17.26 | 11.13 | 15.23 | 20.25 | 11.74 | 5.68 |
| DAAM2 | 34.62 | 28.99 | 19.24 | 24.68 | 31.64 | 21.42 | 12.88 |
| DACH1 | 5.19 | 4.64 | 6.99 | 5.30 | 4.87 | 4.67 | 2.24 |
| DACH1 | 4.08 | 3.87 | 4.22 | 4.44 | 4.60 | 3.86 | 1.91 |
| DISC1 | 3.31 | 2.71 | 1.79 | 3.31 | 2.93 | 1.95 | 1.69 |
| A21 P000047 | | | | | | | |
| DISC1 | 1.99 | 1.62 | 1.39 | 3.3 | 1.82 | 1.15 | 1.05 |
| A24 P83787 | | | | | | | |
| DISC1 | 2.04 | 2.13 | 1.15 | 3.62 | 1.78 | 1.15 | 1.36 |
| A21 P0000050 | | | | | | | |
| EPSTI1 | 1.28 | 1.11 | −1.97 | 1.16 | 1.06 | −1.18 | 1.22 |
| FAM20A | 42.38 | 35.60 | 29.98 | 49.56 | 48.43 | 18.99 | 9.03 |
| A32 P108254 | | | | | | | |
| FAM20A | 21.87 | 18.64 | 19.49 | 24.61 | 24.69 | 10.81 | 5.66 |
| A23 P352952 | | | | | | | |
| GPR124 | −1.27 | −1.22 | −1.16 | −1.26 | −1.38 | −1.28 | −1.01 |
| HCAR2 | 2.61 | 2.96 | 1.83 | 1.53 | 1.46 | 1.85 | 2.01 |
| HP | 20.38 | 17.05 | 13.78 | 16.06 | 15.98 | 12.28 | 3.85 |
| IFI44 | 1.97 | 1.92 | 1.25 | 2.08 | 1.73 | 1.31 | 1.90 |
| IFIT1 | −1.23 | −1.04 | −1.75 | −1.71 | −1.71 | −1.76 | −1.01 |
| IGFBP2 | 14.49 | 12.66 | 7.72 | 11.68 | 12.13 | 9.35 | 5.09 |
| IL10 | 7.01 | 5.77 | 4.95 | 5.95 | 5.40 | 4.90 | 1.91 |
| IL1R1 | 9.66 | 9.49 | 7.58 | 8.23 | 9.12 | 6.61 | 3.82 |
| IL1R1 | 10.91 | 7.62 | 6.97 | 8.90 | 9.08 | 6.05 | 3.93 |
| IL1R2 | 23.07 | 18.09 | 9.14 | 19.90 | 18.26 | 13.36 | 4.09 |
| IL1RN | 3.83 | 3.70 | 3.31 | 3.46 | 3.44 | 2.93 | 2.30 |
| ITGA2B | 3.27 | 3.21 | 3.32 | 3.12 | 2.79 | 3.09 | 2.15 |
| ITGA7 | 13.21 | 11.13 | 8.60 | 14.92 | 16.81 | 9.64 | 3.13 |
| ITGB3 | 3.56 | 2.41 | 2.53 | 3.21 | 3.00 | 3.02 | 1.82 |
| KIF2C | 1.90 | 2.08 | 1.58 | 2.56 | 2.54 | 2.55 | 1.76 |
| LCN15 | 1.24 | 1.42 | 1.47 | 1.29 | 1.38 | 1.10 | −1.52 |
| LCN2 | 2.31 | 2.67 | 2.71 | 3.01 | 2.85 | 3.66 | 1.91 |
| LGALS2 | −4.81 | −3.39 | −1.80 | −6.29 | −4.79 | −2.14 | −2.15 |
| MAP1A | 2.47 | 2.46 | 2.30 | 2.40 | 2.20 | 2.21 | 1.66 |
| METTL7B | 19.19 | 19.74 | 14.88 | 14.65 | 14.85 | 8.00 | 4.33 |
| MMP8 | 4.03 | 3.55 | 4.48 | 6.85 | 6.96 | 9.21 | 3.31 |
| MMP9 | 30.61 | 21.38 | 16.84 | 33.13 | 27.09 | 23.30 | 8.62 |
| MPP3 | 1.43 | 1.22 | 1.96 | 1.66 | 1.56 | 1.64 | 1.76 |
| MRAS | −2.07 | −2.85 | −2.27 | −1.78 | −1.86 | −2.06 | −1.49 |
| MYCL | −2.13 | −2.29 | −1.39 | −2.45 | −2.04 | −1.40 | −1.04 |
| MYL9 | 2.89 | 2.56 | 3.22 | 2.30 | 2.05 | 3.08 | 2.03 |
| NEXN | 2.54 | 2.07 | 1.14 | 2.60 | 2.20 | 1.65 | 1.26 |
| NLRC4 | 5.12 | 4.55 | 4.05 | 4.90 | 5.18 | 3.12 | 2.08 |
| NLRP3 | 1.79 | 1.48 | 1.91 | 1.72 | 1.66 | 1.75 | 1.69 |
| OLAH | 32.24 | 30.02 | 18.21 | 30.43 | 35.43 | 18.81 | 5.40 |
| PCOLCE2 | 31.87 | 20.26 | 15.76 | 46.07 | 36.83 | 17.08 | 5.06 |
| PF4 | 2.21 | 1.66 | 1.59 | 1.89 | 1.50 | 1.39 | 1.17 |
| PLA2G7 | −2.36 | −3.66 | −2.11 | −2.72 | −3.02 | −2.36 | −1.29 |
| PPBP | 1.71 | 1.09 | 1.61 | 1.61 | 1.37 | 1.54 | 1.24 |
| RBP4 | 3.25 | 2.99 | 2.13 | 3.05 | 2.86 | 2.20 | 1.76 |
| RETN | 16.73 | 11.23 | 8.32 | 15.61 | 12.18 | 7.48 | 3.55 |
| RPGRIP1 | −1.30 | −1.06 | −1.01 | −1.74 | −1.46 | −1.08 | 1.49 |
| SELP | 2.92 | 2.52 | 2.71 | 3.31 | 2.72 | 2.53 | 1.68 |
| SLAMF6 | −5.85 | −4.75 | −3.82 | −5.94 | −5.34 | −4.80 | −1.38 |
| SLC39A8 | 1.66 | 1.83 | 1.62 | 2.95 | 2.49 | 1.36 | 1.05 |
| SLC39A8 | 1.84 | 1.49 | 1.35 | 2.09 | 2.16 | 1.33 | 1.10 |
| TDRD9 | 12.54 | 9.68 | 8.48 | 14.69 | 14.74 | 10.01 | 3.96 |
| TGFA | 4.37 | 4.42 | 3.13 | 3.85 | 3.24 | 2.42 | 1.81 |
| TGFBI | −1.26 | −1.30 | −1.09 | −1.47 | −1.30 | −1.04 | 1.16 |
| TMEM37 | 3.83 | 3.78 | 2.45 | 3.36 | 3.99 | 2.93 | 1.72 |
| TNF | 1.04 | 1.09 | 1.15 | 1.17 | 1.24 | 1.21 | 1.29 |
| TREML1 | 2.25 | 2.16 | 2.57 | 2.40 | 2.07 | 2.46 | 1.65 |
| VSTM1 | 2.05 | 2.50 | 2.54 | 2.60 | 2.97 | 2.78 | 1.87 |
| LILRB4 | 3.94 | 4.20 | 3.52 | 4.02 | 3.48 | 3.15 | 3.31 |
| LILRB5 | 6.68 | 5.24 | 2.24 | 5.65 | 6.12 | 5.07 | 2.69 |
| NECAB1 | 1.63 | 1.73 | 1.38 | 1.70 | 1.49 | 1.23 | 1.32 |
| NECAB2 | 3.38 | 4.70 | 4.23 | 1.85 | 2.61 | 3.50 | 1.90 |
| PKD1 A21 | 1.39 | 1.30 | 1.38 | 1.26 | 1.12 | 1.30 | 1.47 |
| P0011417 | | | | | | | |
| PKD1 | 1.45 | 1.43 | 1.37 | 1.04 | 1.02 | 1.02 | 1.16 |
| A21 P0011418 | | | | | | | |
| PKD1 | 1.25 | 1.32 | 1.32 | −1.1 | 1.05 | 1.14 | 1.37 |
| A21 P0011419 | | | | | | | |
| PKHD1 | 1.72 | 1.86 | 1.09 | 1.80 | 1.64 | 1.35 | 1.43 |
| A23 P402187 | | | | | | | |
| PKHD1 | 1.97 | 1.87 | 1.01 | 1.79 | 1.63 | 1.43 | 1.41 |
| A33 P3387420 | | | | | | | |
| PKHD1 | 1.83 | 1.95 | 1.13 | 1.61 | 1.61 | 1.36 | 1.63 |
| A23 P424617 | | | | | | | |
| BCL11B | −6.77 | −6.65 | −3.57 | −5.19 | −4.82 | −3.97 | −2.46 |
| CD160 | −7.07 | −5.68 | −3.58 | −7.20 | −5.83 | −6.05 | −2.24 |
| CD2 | −2.71 | −2.39 | −1.52 | −2.66 | −2.49 | −2.16 | −1.31 |
| FCER1A | −21.37 | −32.23 | −10.36 | −19.22 | −20.68 | −11.38 | −3.32 |
| HLA−DPB1 | −3.67 | −3.40 | −4.00 | −3.28 | −3.12 | −3.06 | −1.86 |
| HLA−DRA | −4.57 | −5.05 | −3.90 | −4.34 | −4.22 | −3.38 | −2.24 |
| ITM2A | −4.69 | −5.18 | −4.13 | −3.79 | −3.91 | −3.15 | −2.67 |
| KLRB1 | −6.68 | −6.59 | −3.09 | −5.33 | −5.70 | −3.82 | −2.42 |
| KLRF1 | −5.83 | −4.86 | −2.46 | −4.55 | −4.95 | −3.82 | −2.44 |
| KLRK1 | −10.84 | −9.50 | −5.69 | −9.74 | −8.63 | −6.89 | −4.47 |
| MX1 | 1.04 | 1.12 | −1.20 | −1.03 | −1.09 | −1.31 | 1.06 |
| NPPC | 2.55 | 2.63 | 1.66 | 2.17 | 1.79 | 1.71 | 1.83 |

Table 5 summarises the fold-changes observed in the amounts of the biomarkers quantified in the different patient samples as compared to the amounts quantified for the healthy control samples.

### EXAMPLE 2: ANALYSIS OF BIOMARKER PERFORMANCE

To further investigate the performance of each biomarker in the diagnosis and monitoring of systemic inflammatory disease, Receiver Operating Characteristic (ROC) analysis was used to investigate the ability of the biomarkers to discriminate between different disease conditions and/or recovery status using the gene expression data obtained from each group of patients.

All ROC curve analysis was performed using R software and the ROCR package using the following commands:

Each data value is assigned a predictor label where negatives (non-infected) are “0” and positives (infected) are “1” and columns are saved as .txt file to be imported into R. -(GBP <- read.table(“GBP.txt”, header=T))

To plot ROC CURVE:

-pred <- prediction(GBP$GBP1, GBP$labels)
-perf <- performance(pred, measure=“tpr”, x.measure=“fpr”)
-plot(perf); abline(0,1) (# This plots FPR (1-SPEC) on the x axis and TPR (==SENS) on the y axis.)

To measure Area Under Curve Value:

-auc.perf <- performance(pred, measure=“auc”) (# generate performance object with AUC)
-auc.perf@y.values (# and extract the AUC value thus)

To plot accuracy and predict optimal accuracy cutoff values (Accuracy is (TP+TN)/(P+N) or (TP+TN)/(TP+FN+FP+FN) which is the total number of True Positives & True Negatives over the sum of the whole population

-acc.perf <- performance(pred, measure=“acc”)
-plot(acc.perf)
-ind=which. max(slot(acc.perf, “y.values”)[[1]])
-acc=slot(acc.perf, “y.values”)[[1]][ind]
-cutoff=slot(acc.perf, “x.values”)[[1]][ind]
-print(c(accuracy=acc, cutoff=cutoff))
-abline(cutoff,1)
library(ROCR)
data(ROCR.simple)
pred <- prediction(ROCR.simple$predictions, ROCR.simple$labels)
perf <- performance(pred,“tpr”,“fpr”)
plot(perf,colorize=TRUE)
abline(0,1, col=“red”)
auc <- performance(pred, “auc”)@y.values[[1]]
legend(0.5,0.4,paste(c(“AUC=”), round(auc,2), sep=“ ”))

ROC analysis as described above was used to determine the sensitivity and specificity with which biomarkers of the invention discriminate between: (i) patients having a systemic inflammatory condition and healthy controls; (ii) patients having sepsis and patients having SIRS; (iii) patients having abdominal sepsis and patients having pulmonary sepsis; and (iv) healthy controls and patients that recover from a systemic inflammatory condition; and (v) patients that recover from a systemic inflammatory condition and patients that do not recover from a systemic inflammatory condition.

### ROC Analysis of the Inflammatory Markers

Table 1 summarises the genes identified as general biomarkers of all systemic inflammatory conditions using gene expression analysis. To demonstrate that these biomarkers permit accurate diagnosis of systemic inflammatory conditions in patients, the “Day 1” gene expression data obtained for all patients was analysed by ROC analysis to determine how well the biomarkers discriminate between patients having a systemic inflammatory condition (including patients having sepsis and patients having SIRS) and healthy controls. ROC analysis of the Day 1 fold change gene expression data was performed as described above.

The ROC curves obtained for the best performing inflammation biomarkers (FAM20A, OLAH and CD177) are shown in FIG. 5. In a ROC curve the true positive rate (Sensitivity) is plotted in function of the false positive rate (100-Specificity) for different cut-off points of a parameter. Each point on the ROC curve represents a sensitivity/specificity pair corresponding to a particular decision threshold. The area under the ROC curve (AUC) is a measure of how well a parameter can distinguish between two diagnostic groups (diseased/normal). A ROC curve plots Sensitivity on the Y axis and 1-specificity on the X axis for a “family” of threshold cut-offs (1-specificity is also known as the False Positive Rate (FPR). A test with perfect discrimination (no overlap in the two distributions) has a ROC curve that passes through the upper left corner (100% sensitivity, 100% specificity). Therefore the closer the ROC curve is to the upper left corner, the higher the overall accuracy of the test (Zweig & Campbell, 1993). AUC and ACC values calculated for the best performing biomarkers are summarised in the table below. AUC values above 0.6 indicate that the biomarker discriminates between the patient populations being tested.

TABLE 6

| | | Area under | | |
|---|---|---|---|---|
| BIO- | PROBE | Curve | Accuracy | |
| MARKER | NUMBER | (AUC) | (ACC) | Cut-off |
| FAM20A | A_32_P108254 | 0.9982143 | 0.9848485 | −4.0993210 |
| FAM20A | A_24_P352952 | 0.9950397 | 0.979798 | −2.818870 |
| OLAH | A_23_P161458 | 0.9634921 | 0.9444444 | −3.0706300 |
| CD177 | A_21_P0011751 | 0.9805556 | 0.9747475 | −4.2479086 |
| CD177 | A_23_P259863 | 0.9876984 | 0.9747475 | −3.9516115 |
| 2 Marker | A_32_P108254 | 0.9882496 | 0.96633 | −3.07063 |
| panel | A_24_P352952 | | | |
| (FAM20A; | A_23_P161458 | | | |
| OLAH) | | | | |
| 3 MARKER | A_32_P108254 | 0.9847222 | 0.9606061 | −3.3482232 |
| PANEL | A_24_P352952 | | | |
| (FAM20A, | A_23_P161458 | | | |
| OLAH; | A_21_P0011751 | | | |
| CD177) | A_23_P259863 | | | |

As demonstrated by the AUC and ACC values reported in the above table, FAM20A, OLAH and CD177 were observed to specifically and sensitively distinguish between healthy control patients (having no systemic inflammatory disease) and patients having a systemic inflammatory condition (ie. those having sepsis or SIRS) when used on their own. The combination of FAM20A and OLAH and the combination of FAM20A, OLAH and CD177 also specifically and sensitively distinguished between patients having a systemic inflammatory condition and healthy controls. These biomarkers may therefore be preferably used in the methods of the invention to diagnose systemic inflammation.

### ROC Analysis of the Sepsis and SIRS Markers

Tables 2 and 3 summarise the genes identified as sepsis biomarkers and SIRS biomarkers using gene expression analysis. To demonstrate that these biomarkers permit accurate diagnosis of sepsis and SIRS (and distinguish between these specific disease conditions), the “Day 1” gene expression data obtained for all disease patients was analysed by ROC analysis to determine how well the biomarkers discriminate between patients having sepsis (including patients having abdominal sepsis and patients having pulmonary sepsis) and patients having SIRS. ROC analysis of the Day 1 fold change gene expression data was performed as described above.

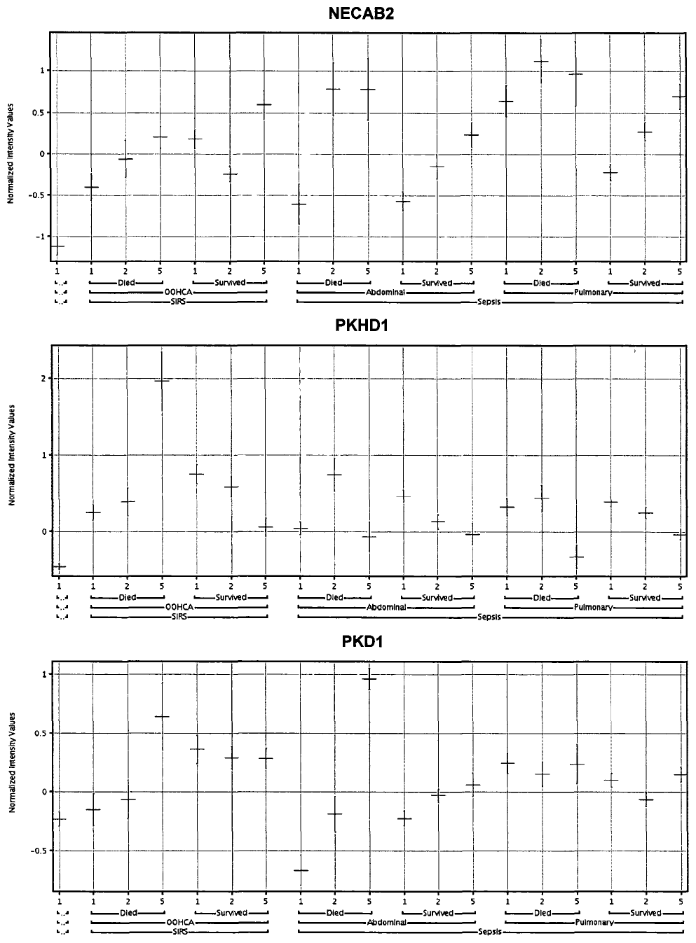

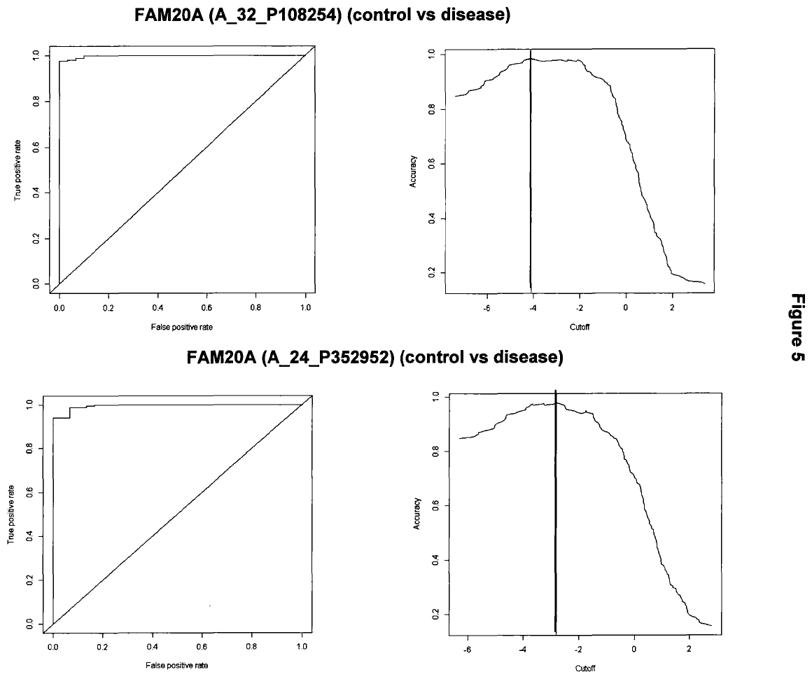

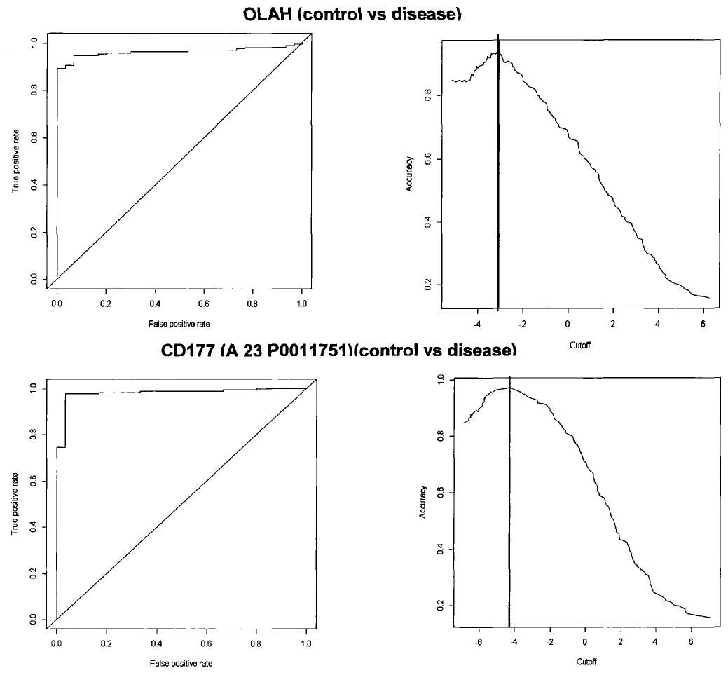

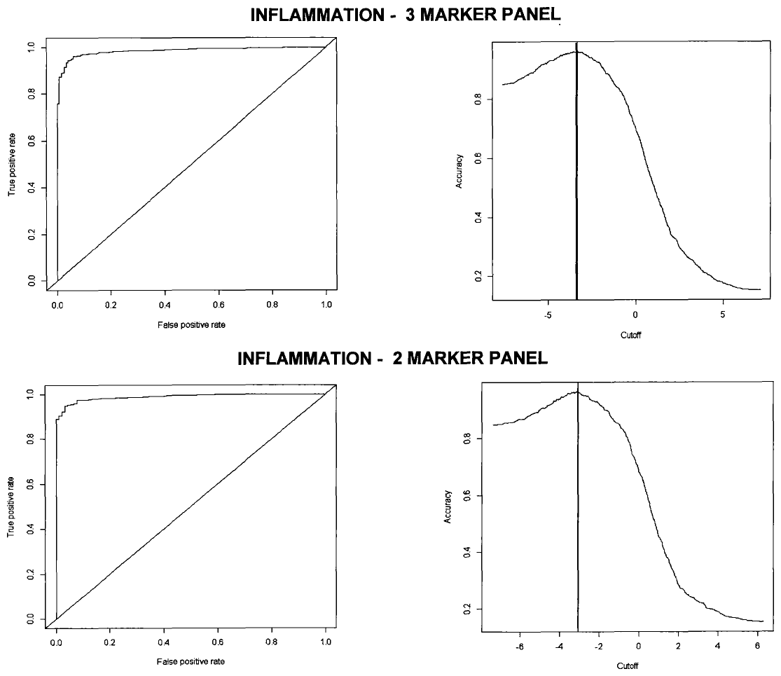

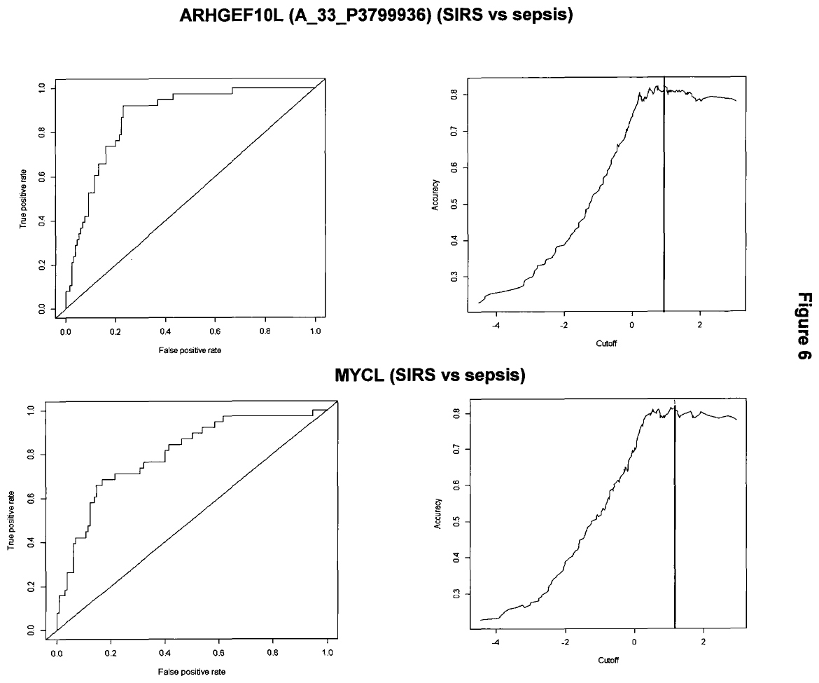

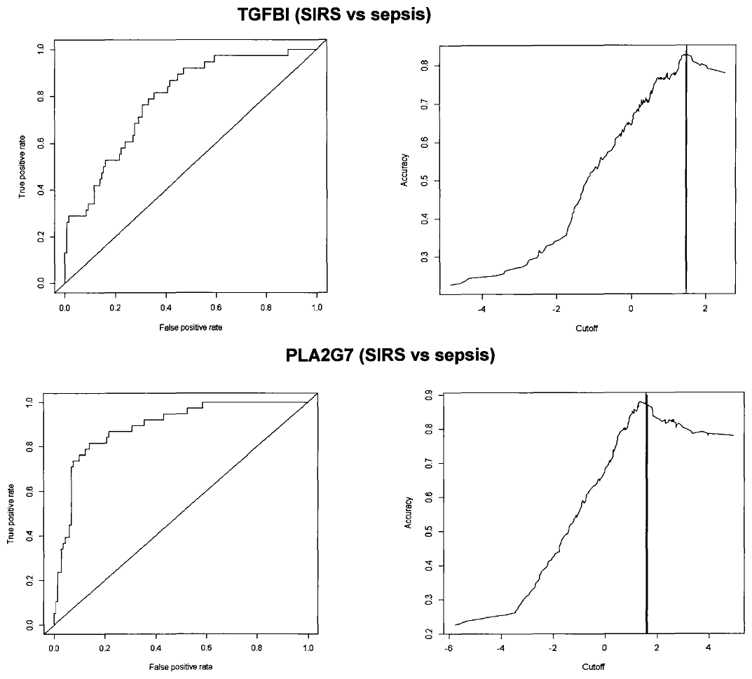

The ROC curves obtained for the best performing biomarkers are shown in FIG. 6. AUC and ACC values calculated for these biomarkers are summarised in the table below.

TABLE 7

| | | Area under | Accuracy | | SIRS/ |
|---|---|---|---|---|---|
| BIOMARKER | PROBE NUMBER | Curve (AUC) | (ACC) | Cut-off | SEPSIS |
| ARGHEF10L | A_33_P3799936 | 0.8684211 | 0.8214286 | 0.9472856 | +/− |
| ARGHEF10L | A_33_P3215575 | 0.8016194 | 0.8154762 | 1.4029250 | +/− |
| MYCL | A_33_P3306068 | 0.8008097 | 0.8154762 | 1.1467919 | +/− |
| TGFBI | A_23_P156327 | 0.7884615 | 0.827381 | 1.461718 | +/− |
| PLA2G7 | A_23_P145096 | 0.890081 | 0.8809524 | 1.5761456 | +/− |
| ITGB3 | A_24_P318656 | 0.8597166 | 0.8392857 | −2.7937140 | −/+ |
| ITGA2B | A_24_P65373 | 0.8321862 | 0.8333333 | −2.05138 | −/+ |
| MYL9 | A_23_P210425 | 0.8234818 | 0.827381 | −2.277507 | −/+ |
| LCN2 | A_23_P169437 | 0.8125506 | 0.8214286 | −2.1682110 | −/+ |
| TREML1 | A_33_P338177 | 0.8074899 | 0.827381 | −2.528779 | −/+ |
| 8 BIOMARKER | ALL PROBES | 0.770304 | 0.7050265 | 0.1868129 | |
| PANEL (PLA2G7 | | | | | |
| REMOVED) | | | | | |
| 9 BIOMARKER | ALL PROBES | 0.7691327 | 0.7065476 | 0.3061080 | |
| PANEL | | | | | |
| SIRS PANEL | A_33_P3799936 | 0.8298785 | 0.825000 | 1.402111 | +/− |
| (ARGHEF10L | A_33_P3215575 | | | | |
| MYCL | A_33_P3306068 | | | | |
| TGFBI | A_23_P156327 | | | | |
| PLA2G7) | A_23_P145096 | | | | |
| SEPSIS PANEL | A_24_P318656 | 0.827417 | 0.8238095 | −2.1682110 | −/+ |
| (ITGB3 | A_24_P65373 | | | | |
| ITGA2B | A_23_P210425 | | | | |
| MYL9 | A_23_P169437 | | | | |
| LCN2 | A_33_P338177 | | | | |
| TREML1) | | | | | |

As demonstrated by the AUC and ACC values reported in the above table, the following biomarkers were observed to specifically and sensitively distinguish between patients having sepsis and patients having SIRS when used on their own:
(i) The sepsis biomarkers: LCN2, ITGA2B, MYL9, ITGB3, and TREML1; and
(ii) The SIRS biomarkers: TGFBI, PLA2G7, MYCL, ARHGEF10L

The combination of all sepsis biomarkers (ITGB3, ITGA2B, MYL9, LCN2 and TREML1), the combination of all SIRS biomarkers (ARGHEF10L, MYCL, TGFB1 and PLA2G7), and the combination of all sepsis and all SIRS biomarkers also specifically and sensitively distinguished between patients having sepsis and patients having SIRS. These biomarkers may therefore be preferably used on their own or in combination in the methods of the invention to diagnose sepsis or SIRS, and to distinguish between sepsis and SIRS.

### ROC Analysis of Abdominal Sepsis and Pulmonary Sepsis Markers

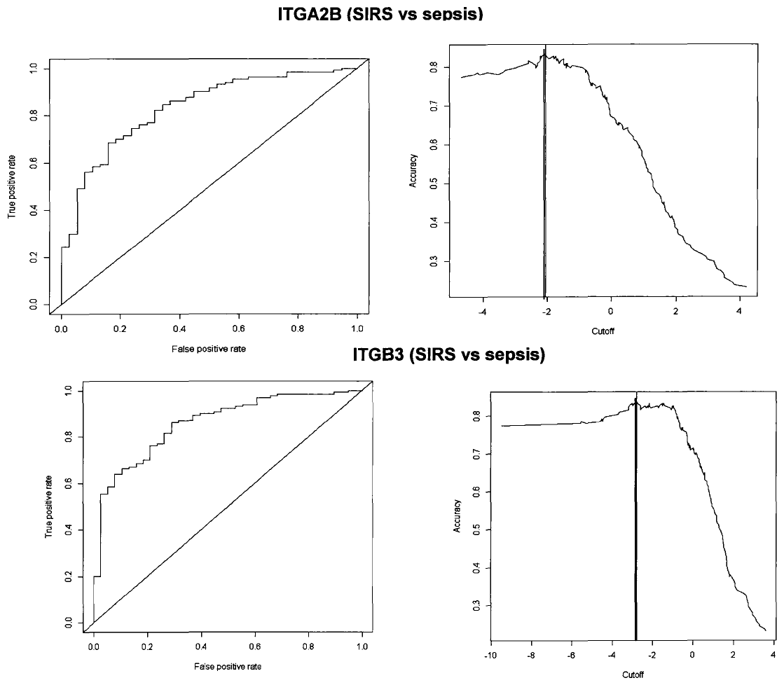

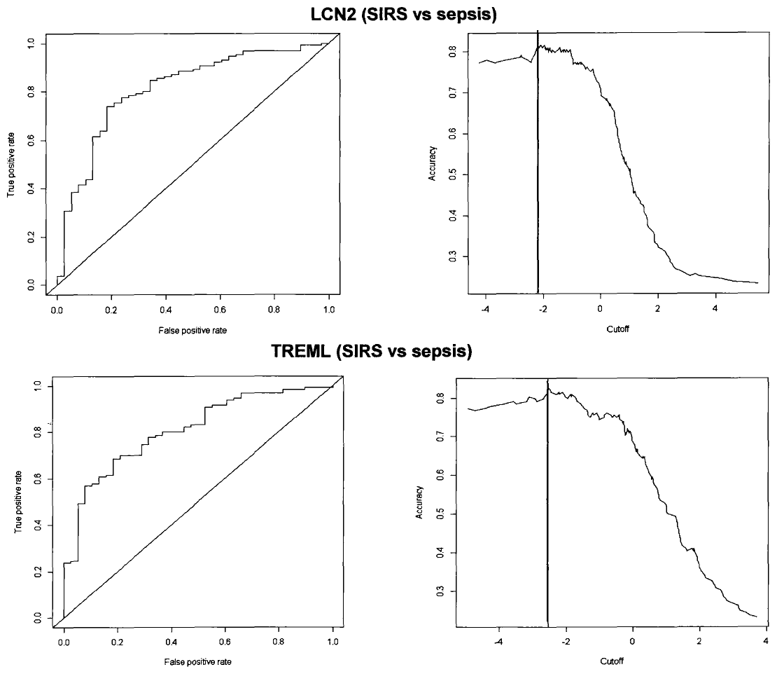

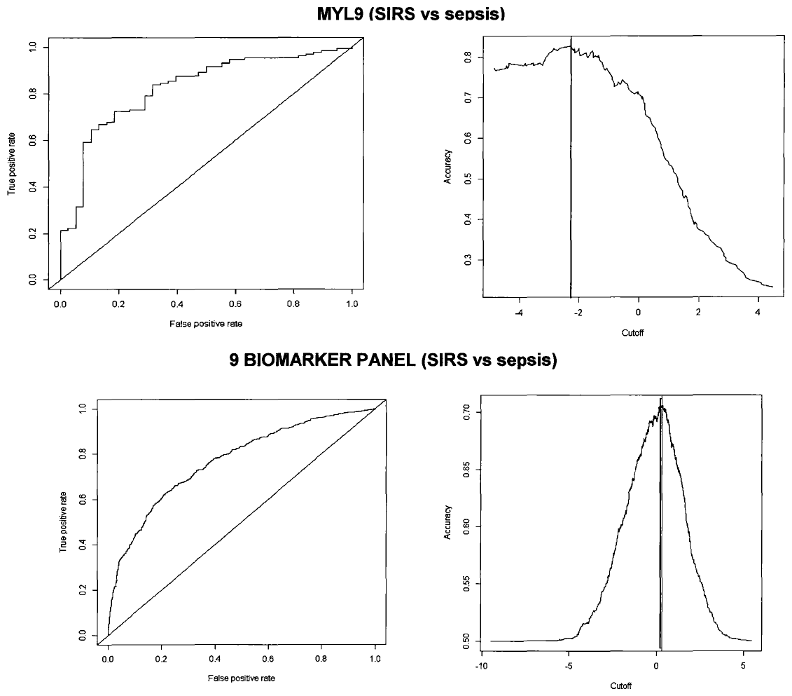

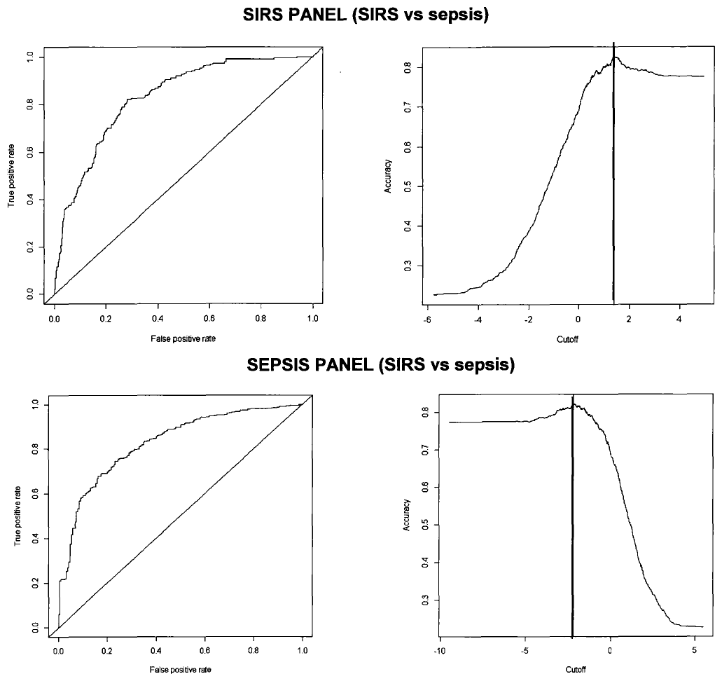

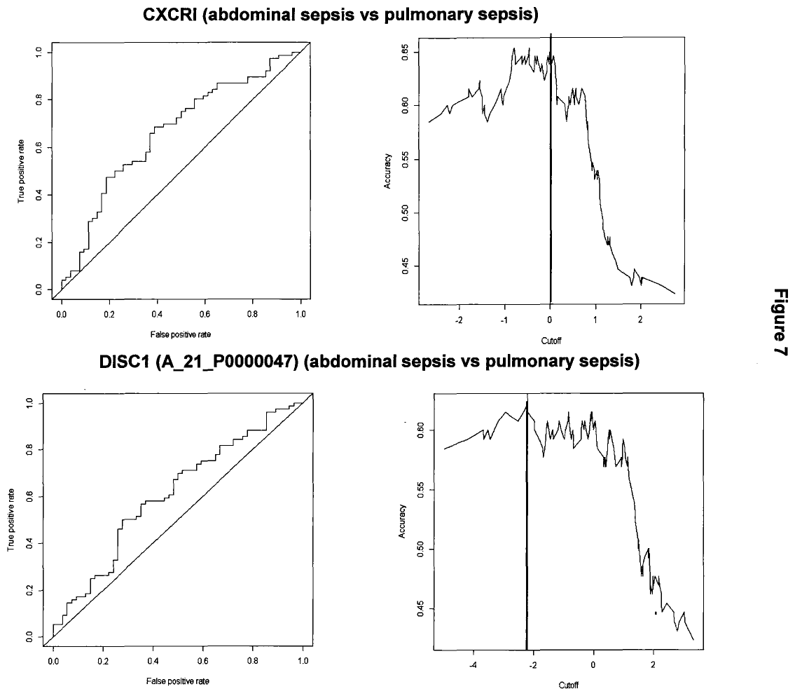

Table 3 summarises the genes identified as biomarkers of abdominal sepsis and pulmonary sepsis using gene expression analysis. To demonstrate that these biomarkers permit accurate diagnosis of abdominal and pulmonary sepsis (and distinguish between these specific disease conditions), the “Day 1” gene expression data obtained for all sepsis patients was analysed by ROC analysis to determine how well the biomarkers discriminate between patients having abdominal sepsis and patients having pulmonary sepsis. ROC analysis of the Day 1 fold change gene expression data was performed as described above. The ROC curves obtained for the best performing biomarkers are shown in FIG. 7. AUC and ACC values calculated for abdominal sepsis and pulmonary sepsis biomarkers are summarised in the table below.

TABLE 8

| | | Area under | Accuracy | | Abdominal/ |
|---|---|---|---|---|---|
| BIOMARKER | PROBE NUMBER | Curve (AUC) | (ACC) | Cut-off | Pulmonary |
| CXCR1 | A_23_P67932 | 0.6630117 | 0.6538462 | 0.00 | −/+ |
| DISC1 | A_21_P0000047 | 0.6079435 | 0.6230769 | −2.2045078 | −/+ |
| DISC1 | A_24_P83787 | 0.6028265 | 0.6230769 | −0.8441138 | −/+ |
| DISC1 | A_21_P0000050 | 0.6084308 | 0.6307692 | −0.8969114 | −/+ |
| HCAR2 | A_23_P329924 | 0.7022417 | 0.6769231 | −1.4589972 | −/+ |
| SLC39A8 (ZIP8) | A_23_P41424 | 0.7748538 | 0.7538462 | 1.1714854 | +/− |
| C1QA | A_24_P222655 | 0.7351365 | 0.7230769 | 1.4086328 | +/− |
| C1QB | A_23_P137366 | 0.7302632 | 0.6846154 | 2.3654090 | +/− |
| C1QC | A_23_P125977 | 0.7412281 | 0.7153846 | 2.5970287 | +/− |
| MRAS | A_24_P88850 | 0.7422027 | 0.7153846 | 0.1304908 | +/− |
| TMEM37 | A_33_P3289296 | 0.7368421 | 0.7230769 | 1.1748223 | +/− |
| Pulmonary | A_23_P67932 | 0.6224269 | 0.6215385 | −0.6175871 | −/+ |
| sepsis 3 Marker | A_21_P0000047 | | | | |
| panel | A_24_P83787 | | | | |
| (CXCR1, DISC1, | A_21_P0000050 | | | | |
| HCAR2) | A_23_P329924 | | | | |
| Pulmonary | A_23_P67932 | 0.6800682 | 0.6576923 | 0.000000 | −/+ |
| sepsis 2 Marker | A_23_P329924 | | | | |
| Panel (DISC1 | | | | | |
| REMOVED) | | | | | |
| Abdominal | A_23_P41424 | 0.7473468 | 0.7051282 | 1.0980682 | +/− |
| sepsis 3 Marker | A_24_P222655 | | | | |
| Panel | A_23_P125977 | | | | |
| (SLC39A8, CIQA, | | | | | |
| CIQC) | | | | | |
| Abdominal vs | A_23_P67932 | 0.6471384 | 0.61730769 | 0.04587579 | +/− |
| pulmonary 6 | A_21_P0000047 | | | | |
| marker panel | A_24_P83787 | | | | |
| (CXCR1, DISC1, | A_21_P0000050 | | | | |
| HCAR2, | A_23_P329924 | | | | |
| SLC39A8, CIQA, | A_23_P41424 | | | | |
| CIQC) | A_24_P222655 | | | | |
| | A_23_P125977 | | | | |
| Abdominal vs | A_23_P67932 | 0.6841542 | 0.6415385 | 0.6422954 | +/− |
| pulmonary 5 | A_23_P329924 | | | | |
| marker panel | A_23_P41424 | | | | |
| (DISC1 removed) | A_24_P222655 | | | | |
| | A_23_P125977 | | | | |
| Abdominal | A_23_P41424 | 0.7356204 | 0.6858974 | 1.1297174 | +/− |
| sepsis 6 Marker | A_24_P222655 | | | | |
| panel (SLC39A8, | A_23_P125977 | | | | |
| CIQA, CIQB, | A_24_P88850 | | | | |
| CIQC, MRAS, | A_33_P3289296 | | | | |
| TMEM37) | A_23_P137366 | | | | |

As demonstrated by the AUC and ACC values reported in the above table, the following biomarkers were observed to specifically and sensitively distinguish between patients having abdominal sepsis and patients having pulmonary sepsis when used on their own or in combination:
(i) The abdominal sepsis biomarkers: CXCR1, DISC1, HCAR2; and
(ii) The pulmonary sepsis biomarkers: SLC39A8, CIQA, CIQB, CIQC, MRAS, TMEM37.

These biomarkers may therefore be preferably used in the methods of the invention to diagnose and distinguish between abdominal sepsis and pulmonary sepsis.

### ROC Analysis of Prognosis Biomarkers for Monitoring Disease

When investigating gene expression patterns in patients that survived a systemic inflammatory condition and were deemed suitable for discharge from a high dependency unit (e.g. into a low dependency unit), the present inventors identified various biomarkers (summarised in Table 1) that altered in abundance in disease patients compared to healthy controls, but which returned towards their normal healthy levels as the patients recovered from disease. The inventors identified that these biomarkers could be used to determine the prognosis of a patient with a systemic inflammatory condition, and may be used to monitor the effectiveness of treatment in a patient or determine whether a patient is suitable for discharge.

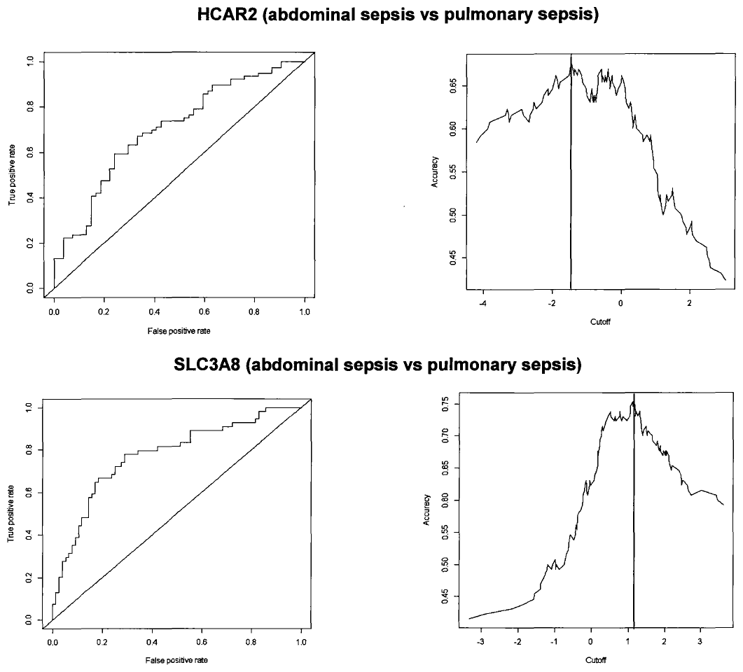

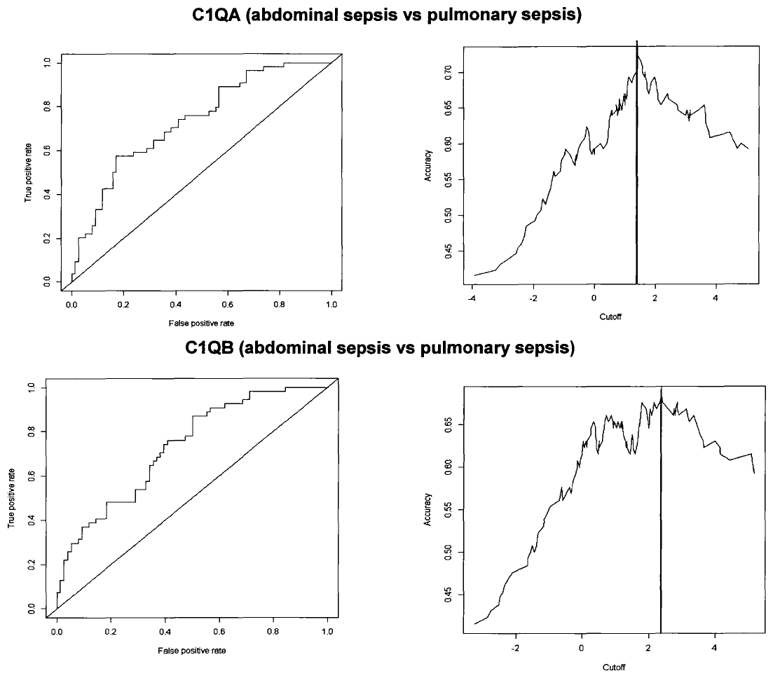

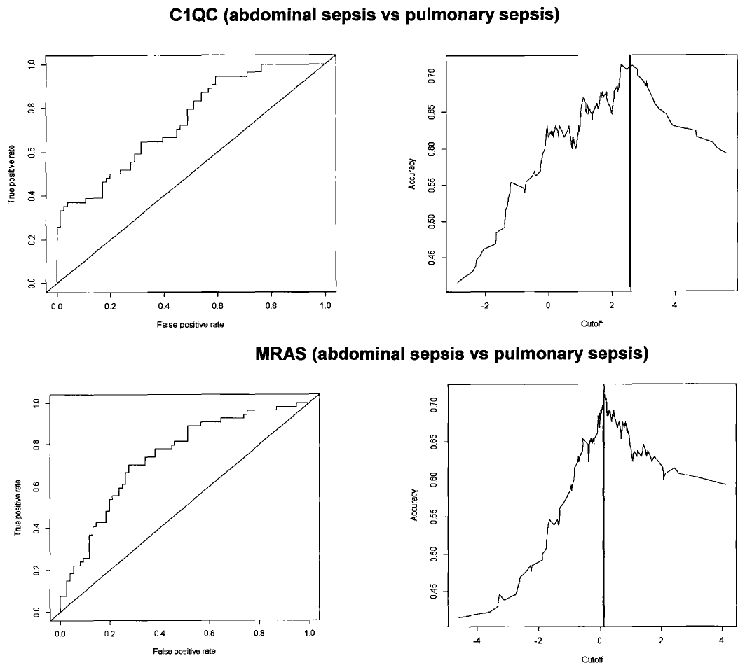

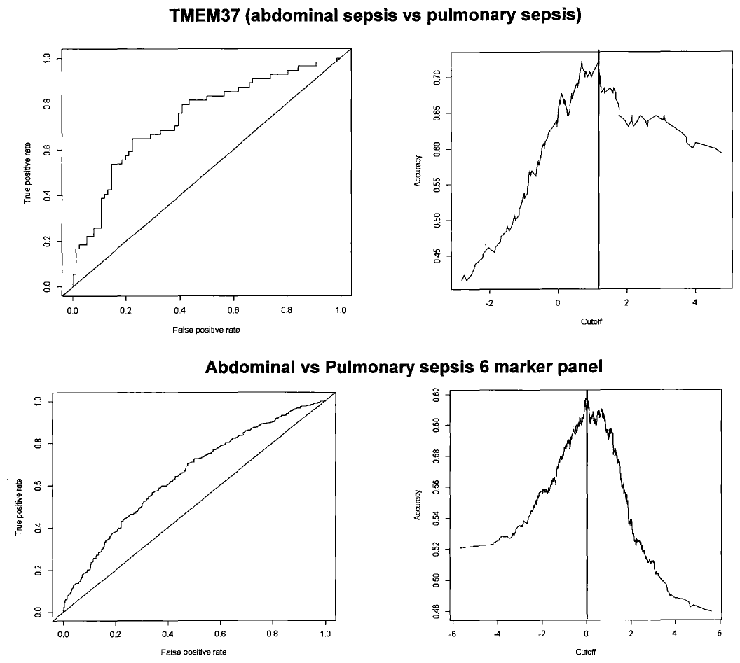

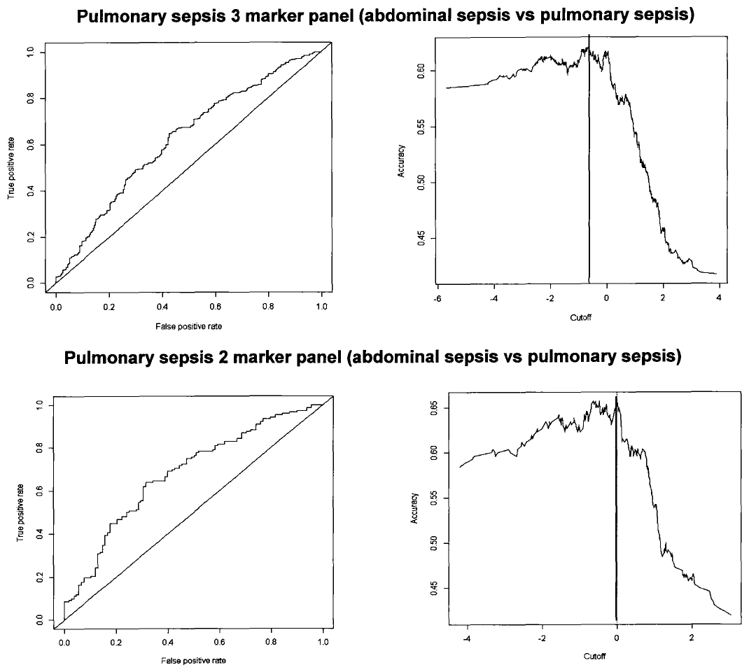

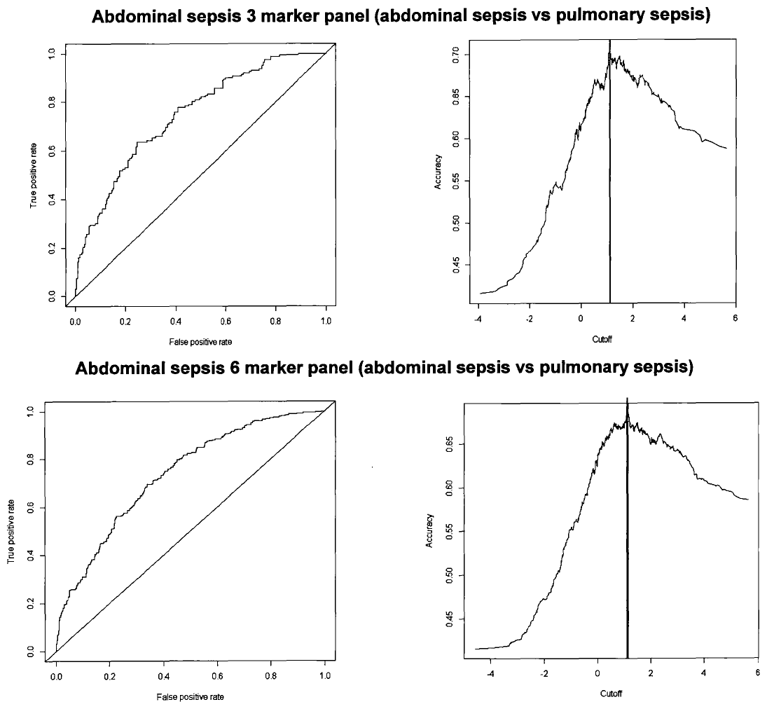

To demonstrate that these ‘prognosis’ biomarkers can be used to monitor the recovery of a patient from a systemic inflammatory condition, ROC analysis was performed to investigate over time the ability of the biomarkers to discriminate between healthy controls and patients that survived a systemic inflammatory condition. AUC values were calculated using the “Day 1”, “Day 5” and “discharge” gene expression data, and are summarised below. An AUC value close to 1 indicates that the biomarkers discriminate well between the healthy controls and disease patient populations, whilst an AUC value close to 0.5 indicates that the two populations cannot be reliably distinguished. Representative ROC curves are plotted for PKHD1 biomarker in FIG. 8.

TABLE 9

| BIO- | SURVIVAL vs | Accuracy | | |
|---|---|---|---|---|
| MARKER | DEATH | (AUC) | (ACC) | Cut-off |
| FCER1A | Control (1) vs | 0.8681818 | 0.8269231 | 3.0441122 |
| Probe: A23 | OOHCA | | | |
| P103765 | survived day 0 | | | |
| | Control (1) vs | 0.92 | 0.950000 | 2.322179 |
| | OOHCA | | | |
| | SURVIVED day 5 | | | |
| | Control vs OOHCA | 0.9857143 | 0.972973 | 2.322179 |
| | SURVIVED | | | |
| | DISCHARGE | | | |
| | Control (1) vs | 0.999187 | 0.9859155 | 2.3221793 |
| | Abdominal sepsis | | | |
| | survived day 0 | | | |
| | Control (1) vs | 0.9964912 | 0.9795918 | 2.3221793 |
| | Abdominal sepsis | | | |
| | survived day 5 | | | |
| | Control vs | 0.9461538 | 0.8837209 | 2.7308435 |
| | Abdominal | | | |
| | survived discharge | | | |
| | Control (1) vs | 0.9798851 | 0.9545455 | 2.7308435 |
| | pulmonary sepsis | | | |
| | survived day 0 | | | |
| | Control (1) vs | 0.9825 | 0.9571429 | 2.7308435 |
| | pulmonary sepsis | | | |
| | survived day 5 | | | |
| | Control vs | 0.8946667 | 0.8545455 | 2.8394332 |
| | pulmonary | | | |
| | sepsis survived | | | |
| | discharge | | | |
| BCL11B | Control vs OOHCA | 0.9409091 | 0.9423077 | 1.3860502 |
| Probe A23 | SURVIVAL DAY 0 | | | |
| P205738 | Control vs OOHCA | 1 | 1 | 1.38605 |
| | SURVIVAL DAY 5 | | | |
| | Control vs OOHCA | 0.9904762 | 0.972973 | 1.585580 |
| | SURVIVAL | | | |
| | DISCHARGE | | | |
| | Control vs | 1 | 1 | 1.38605 |
| | Abdominal | | | |
| | survived day 0 | | | |
| | Control vs | 0.9964912 | 0.9795918 | 1.5855803 |
| | Abdominal | | | |
| | survived day 5 | | | |
| | Control vs | 0.9923077 | 0.9534884 | 1.6249652 |
| | Abdominal | | | |
| | survived discharge | | | |
| | Control vs | 0.9936782 | 0.9772727 | 1.5855803 |
| | pulmonary | | | |
| | survived day 0 | | | |
| | Control vs | 0.9983333 | 0.9857143 | 1.5855803 |
| | pulmonary | | | |
| | survived day 5 | | | |
| | Control vs | 0.9613333 | 0.8909091 | 1.6249652 |
| | pulmonary | | | |
| | survived discharge | | | |
| PKHD1 | Control (1) vs | 0.8621212 | 0.8269231 | 0.2411327 |
| Probe A33 | OOHCA | | | |
| P3387420 | survived day 0 | | | |
| | Control (1) vs | 0.7266667 | 0.8250000 | 0.8070955 |
| | OOHCA | | | |
| | survived day 5 | | | |
| | Control (1) vs | 0.5 | 0.8108108 | inf |
| | OOHCA | | | |
| | survived day 5 | | | |
| | Control (1) vs | 0.7845528 | 0.73239437 | 0.05443954 |
| | Abdominal | | | |
| | survived day 0 | | | |
| | Control (1) vs | 0.5350877 | 0.6938776 | 0.5116310 |
| | Abdominal | | | |
| | survived day 5 | | | |
| | Control (1) vs | 0.6871795 | 0.8139535 | 0.4272528 |
| | Abdominal | | | |
| | survived discharge | | | |
| | Control vs | 0.7781609 | 0.7613636 | −0.4943862 |
| | Pulmonary | | | |
| | survived day 0 | | | |
| | Control vs | 0.6725 | 0.6714286 | −0.3561888 |
| | Pulmonary | | | |
| | survived day 5 | | | |
| | Control vs | 0.6013333 | 0.6727273 | 0.5358357 |
| | Pulmonary | | | |
| | survived discharge | | | |
| KLRB1 | Control vs OOHCA | 0.9818182 | 0.9615385 | 1.0817766 |
| Probe A23 | SURVIVED DAY 0 | | | |
| P99275 | Control vs OOHCA | 0.99 | 0.975000 | 1.081777 |
| | SURVIVED DAY 5 | | | |
| | Control vs OOHCA | 0.9285714 | 0.9189189 | 0.4236860 |
| | SURVIVED | | | |
| | discharge | | | |
| | Control vs | 0.9608333 | 0.9142857 | 1.4716887 |
| | abdominal | | | |
| | survived day 0 | | | |
| | Control vs | 0.98 | 0.960000 | 1.081777 |
| | abdominal | | | |
| | survived day 5 | | | |
| | Control vs | 0.8769231 | 0.8604651 | 1.0817766 |
| | abdominal | | | |
| | survived discharge | | | |
| | Control vs | 0.9816092 | 0.9431818 | 1.0817766 |
| | pulmonary | | | |
| | survived day 0 | | | |
| | Control vs | 0.9725 | 0.9285714 | 1.4716887 |
| | pulmonary | | | |
| | survived day 5 | | | |
| | Control vs | 0.8826667 | 0.8727273 | 1.1410170 |
| | pulmonary | | | |
| | survived discharge | | | |
| LILRB5 | Control vs OOHCA | 0.8166667 | 0.7884615 | −0.7073317 |
| Probe A23 | SURVIVED DAY 0 | | | |
| P4773 | Control vs OOHCA | 0.9266667 | 0.9250000 | 0.2927966 |
| | SURVIVED DAY 5 | | | |
| | Control vs OOHCA | 0.8190476 | 0.8378378 | 0.7142091 |
| | SURVIVED | | | |
| | discharge | | | |
| | Control vs | 0.935 | 0.9142857 | −1.1843534 |
| | abdominal | | | |
| | survived day 0 | | | |
| | Control vs | 0.9035088 | 0.8367347 | −0.2738576 |
| | abdominal | | | |
| | survived day 5 | | | |
| | Control vs | 0.8435897 | 0.8604651 | −0.2342336 |
| | abdominal | | | |
| | survived discharge | | | |
| | Control vs | 0.904023 | 0.8522727 | −1.7467065 |
| | pulmonary | | | |
| | survived day 0 | | | |
| | Control vs | 0.8891667 | 0.8714286 | −1.1859131 |
| | pulmonary | | | |
| | survived day 5 | | | |
| | Control vs | 0.8026667 | 0.7636364 | −1.0275340 |
| | pulmonary | | | |
| | survived discharge | | | |

As demonstrated in the table above, the AUC values calculated for the biomarkers are observed to change over time as the patient recovers from the systemic inflammatory condition, shifting from ‘1’ or close to ‘1’ (for the samples taken at an early stage of the disease, such as at day 0) towards ‘0.5’ (for the samples taken at day 5 or on discharge). This indicates that the biomarkers become less able to discriminate between healthy controls and disease patients as the patient recovers (indicating that the biomarker profile of the patient becomes more representative of a healthy control). For example, when investigating the gene expression data obtained for PKDH1 in the patients with SIRS, the AUC value for the ‘day 0’ sample is 0.86 indicating that the patient with SIRS can be readily distinguished from a healthy control. By ‘day 5’ the AUC value has dropped to 0.73, indicating that there is less distinction between the patient and a healthy control. Upon discharge, the AUC value has dropped to 0.5 indicating that the biomarker profile of the patient cannot be distinguished from that observed for a healthy control. By investigating the levels of these biomarkers in patients having a systemic inflammatory condition, the disease status of the patient may be monitored to determine whether the disease is progressing towards a more severe form of the disease or regressing towards normalcy.

Surprisingly, the inventors observed that for many of the prognosis biomarkers tested, the AUC values calculated for the “discharge” samples did not drop all the way to ‘0.5’ but remained somewhere between 1 and 0.5. This indicates that patients are being discharged before they are fully immunologically recovered (ie. before their biomarker profiles are representative of a healthy control). By monitoring a patient using the ‘prognosis’ biomarkers of the invention, it is possible to determine more accurately when a patient has recovered fully from a systemic inflammatory condition, and can be safely discharged.

### ROC Analysis of Survival Biomarkers

Table 4 summarises the genes identified as survival biomarkers for determining whether a patient with a systemic inflammatory condition is suitable for discharge from medical care. These markers can be used to predict whether a patient undergoing treatment for a systemic inflammatory condition is likely to survive.

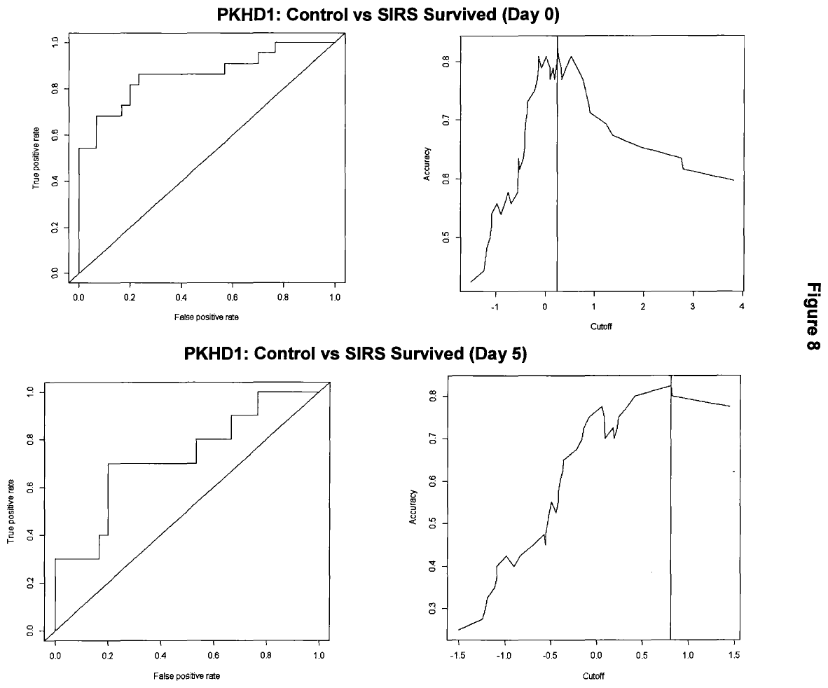

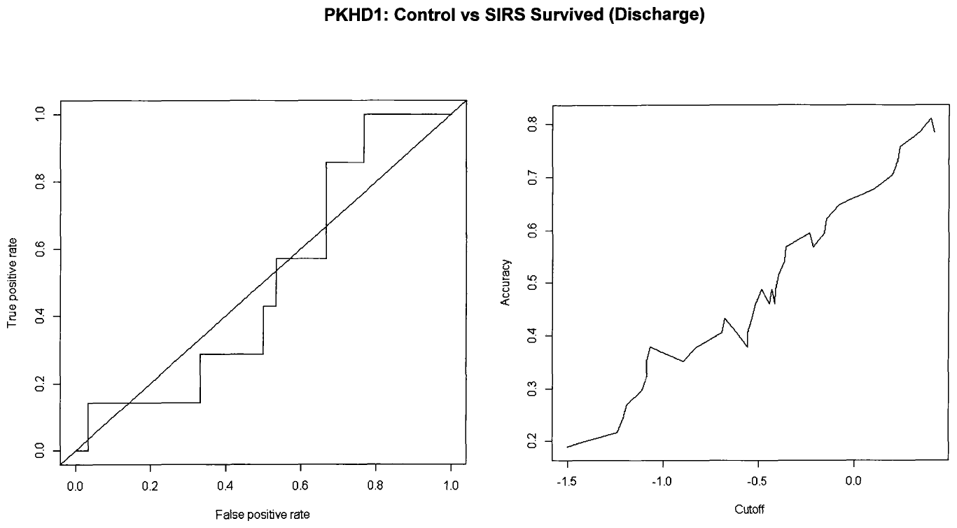

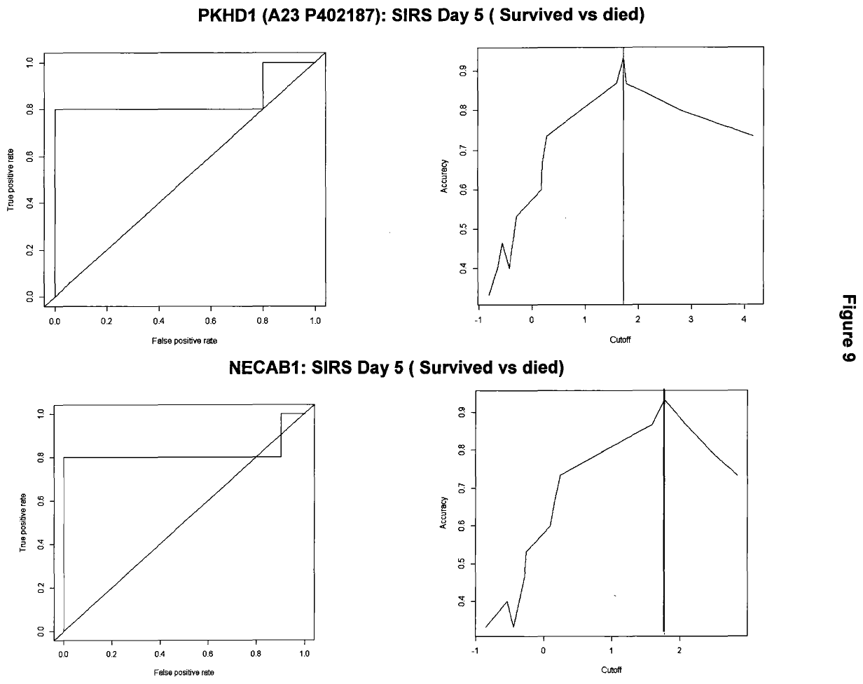

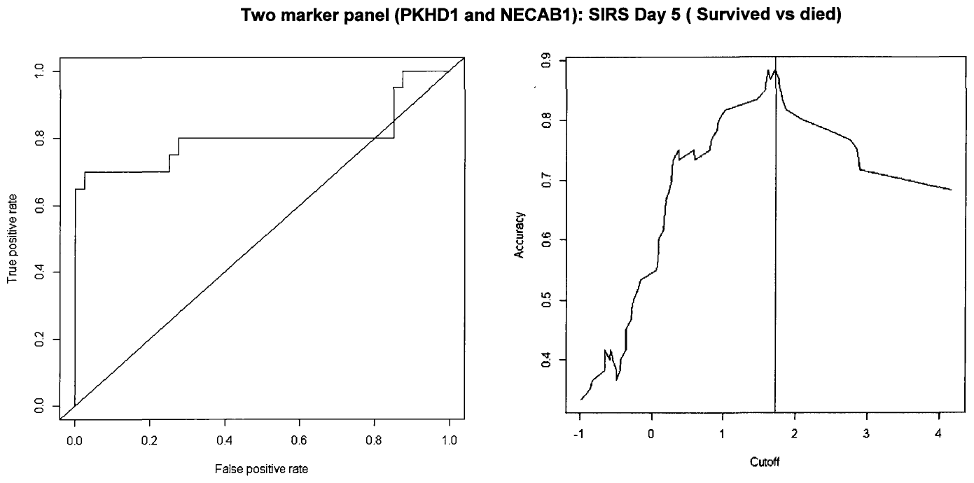

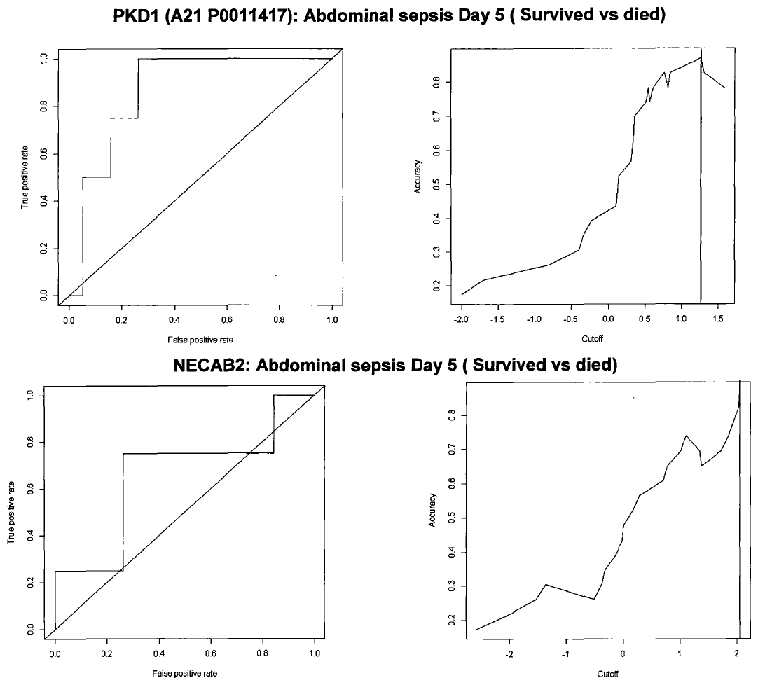

To demonstrate that these biomarkers accurately predict survival, the “Day 5” gene expression data obtained for all patients was analysed by ROC analysis to determine how well the biomarkers discriminate between patients having a systemic inflammatory condition that survive and those do not survive. ROC analysis of the Day 5 fold change gene expression data was performed as described above. The ROC curves obtained for the best performing biomarkers are shown in FIG. 9. AUC and ACC values calculated for the survival biomarkers are summarised in the table below.

TABLE 10
Biomarkers for predicting survival from SIRS

| BIO- | PROBE | Area under | Accuracy | | SURVIVAL |
|---|---|---|---|---|---|
| MARKER | NUMBER | Curve (AUC) | (ACC) | Cut-off | vs DEATH |
| PKHD1 | A_23_P402187 | 0.84 | 0.9333333 | 1.7156901 | +/− |
| PKHD1 | A_33_P3387420 | 0.76 | 0.8666667 | 1.6200123 | +/− |
| PKHD1 | A_23_P424617 | 0.76 | 0.8666667 | 1.8112721 | +/− |
| NECAB1 | A_24_P944756 | 0.82 | 0.9333333 | 1.7683950 | +/− |
| 2 MARKER | A_23_P402187 | 0.80125 | 0.8833333 | 1.7156901 | +/− |
| PANEL | A_33_P3387420 | | | | |
| | A_23_P424617 | | | | |
| | A_24_P944756 | | | | |

TABLE 11
Biomarkers for predicting survival from abdominal sepsis

| | | Area | | | SUR- |
|---|---|---|---|---|---|
| | | under | | | VIVAL |
| BIO- | PROBE | Curve | Accuracy | | vs |
| MARKER | NUMBER | (AUC) | (ACC) | Cut-off | DEATH |
| PKD1 | A_21_P0011417 | 0.8684211 | 0.8695652 | 1.2567754 | +/− |
| PKD1 | A_21_P0011418 | 0.7105263 | 0.8695652 | 2.0439925 | +/− |
| PKD1 | A_21_P0011419 | 0.7368421 | 0.8695652 | 2.4574770 | +/− |
| NECAB2 | A_23_P66011 | 0.6578947 | 0.8695652 | 2.0517770 | +/− |
| 2 | A_21_P0011417 | 0.6533333 | 0.8086957 | 2.0439925 | +/− |
| MARKER | A_21_P0011418 | | | | |
| PANEL | A_21_P0011419 | | | | |
| | A_23_P66011 | | | | |

TABLE 12
Biomarkers for predicting survival from pulmonary sepsis

| BIO- | PROBE | Area under | Accuracy | | SURVIVAL |
|---|---|---|---|---|---|
| MARKER | NUMBER | Curve (AUC) | (ACC) | Cut-off | vs DEATH |
| PKHD1 | A_33_P3387420 | 0.7 | 0.8695652 | INF | −/+ |
| LILRB5 | A_23_P4773 | 0.7291667 | 0.8695652 | INF | −/+ |
| 2 MARKER | A_33_P3387420 | 0.7208333 | 0.8695652 | INF | −/+ |
| PANEL (PKHD1 | A_23_P4773 | | | | |
| and LILRB5) | | | | | |

As demonstrated by the AUC and ACC values reported in the above tables, the following biomarkers (used alone or in combination) were observed to specifically and sensitively distinguish between patients having a systemic inflammatory condition that made a full recovery and those that did not:
(i) Survival markers for predicting recovery from SIRS: PKHD1 and NECAB1
(ii) Survival markers for predicting recovery from abdominal sepsis: PKD1 and NECAB2; and
(iii) Survival markers for predicting recovery from pulmonary sepsis: PKHD1 and LILRB5.

These biomarkers may therefore be preferably used alone or in combination in the methods of the invention to determine whether a patient is suitable for discharge from medical care.

### EXAMPLE 3: QUANTIFICATION OF PROTEIN BIOMARKERS

To further investigate the biomarkers of systemic inflammation identified by gene expression analysis, a subset of the biomarkers was selected for further analysis by ELISA. Protein quantification by ELISA was performed to investigate the abundance of specific biomarkers in whole lysed blood obtained from patients at day 1 and day 5 post admittance to an intensive care unit (as described above for Example 1).

The biomarkers chosen for further analysis were: (i) the pulmonary sepsis biomarker DISC1; (ii) the abdominal sepsis biomarker SLC39A8; (iii) the SIRS biomarker GPR124; and (iv) the survival marker NECAB1 which is used to predict survival from SIRS.

### ELISA Protein Quantification:

Blood samples were collected from patients and processed as described in Example 1. 2 ml of blood sample was mixed with 8 ml of cell lysis buffer at a 1:5 dilution. The cell lysis buffer was purchased from Invitrogen (NP40 cell lysis buffer: 50 mM Tris, pH 7.4, 250 mM NaCl, 5 mM EDTA, 50 mM NaF, 1 mM Na3VO4, 1% Nonidet P40 (NP40), and 0.02% NaN3, supplemented with 1 mM PMSF and protease inhibitor cocktail). The samples were incubated on ice for 60 minutes. The samples were then centrifuged at 13000 rpm for 30 minutes to pellet debris. The supernatant was removed and passed through a 0.22 μm syringe filter. The cell free supernatant was transferred to a fresh tube and stored at −80° C.

ELISA assays were performed on neat or diluted lysed blood samples using commercial ELISA kits.

DISC1 was quantified using ELISA kit MBS9343138, NECAB1 was quantified using ELISA kit MBS9338711, and SLC39A8 was quantified using ELISA kit MBS9381303. Briefly, 50 μl of prepared blood sample was added to the sample well of a microelisa stripplate plate. In addition, 50 μl of each standard was added to the standard wells and 50 μl of sample diluent was added to each blank/control well of the plate. 100 μl of HRP-conjugate reagent was added to each well and incubated for 60 minutes at 37° C. The plate was washed 4 times, and developed by adding 50 μl Chromagen Solution A and 50 μl Chromagen solution B and incubating for 15 minutes at 37° C. 50 μl stop solution was added to the wells, and the optical density at 450 nm was read.

GPR124 was quantified using ELISA kit MBS909585. Briefly, 100 μl of prepared blood sample was added to the sample well of an ELISA plate. In addition, 100 μl of each standard was added to the standard wells and 100 μl of sample diluent was added to each blank/control well of the plate. The plate was incubated for 2 hours at 37° C. 100 μl of biotin-antibody was added to each well and incubated for 60 minutes at 37° C. The plate was washed 3 times. 100 μl of HRP-avidin was added to each well and incubated for 1 hour at 37° C. The plate washed 5 times. 90 μl TMB substrate was added and the plate was incubated for 15-30 minutes at 37° C. 50 μl stop solution was added to the wells, and the optical density at 450 nm was read.

### ROC Analysis of Protein Quantification Data:

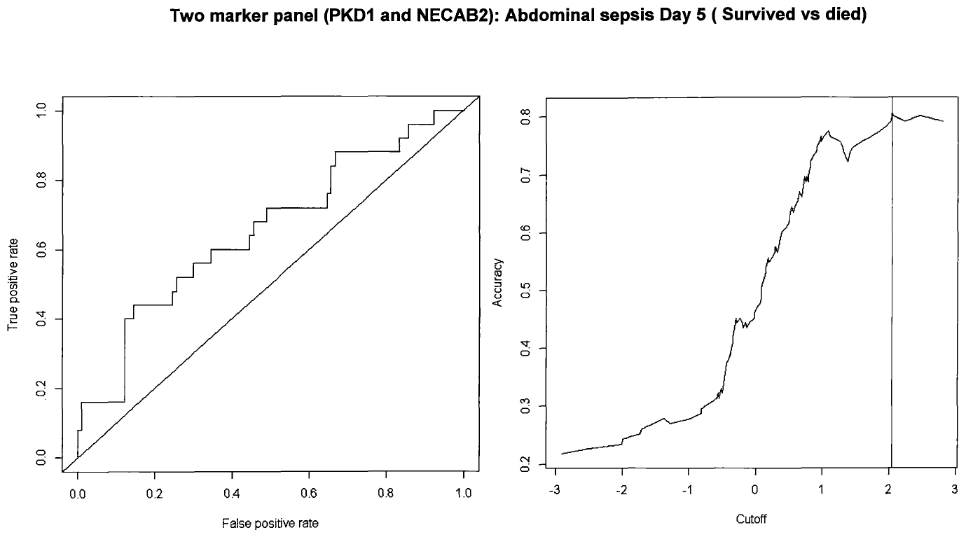

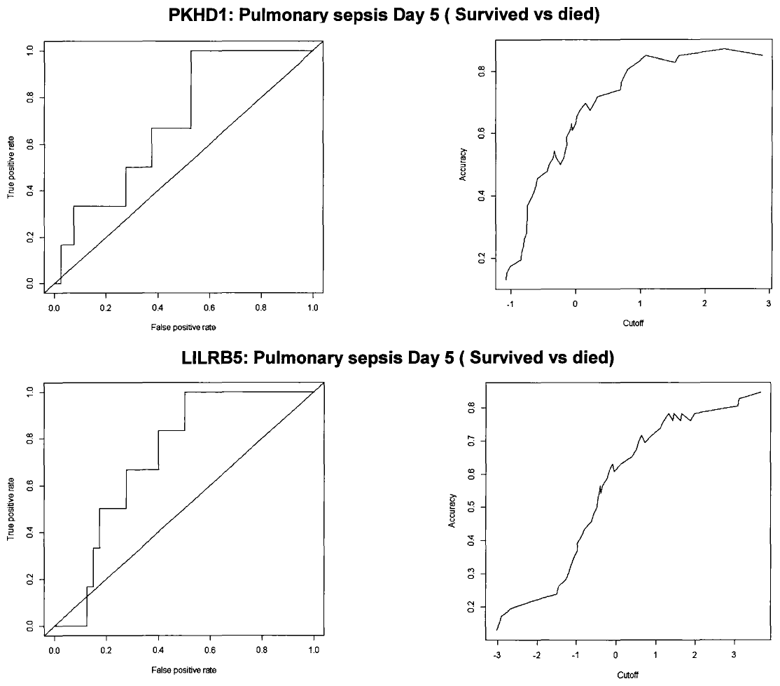

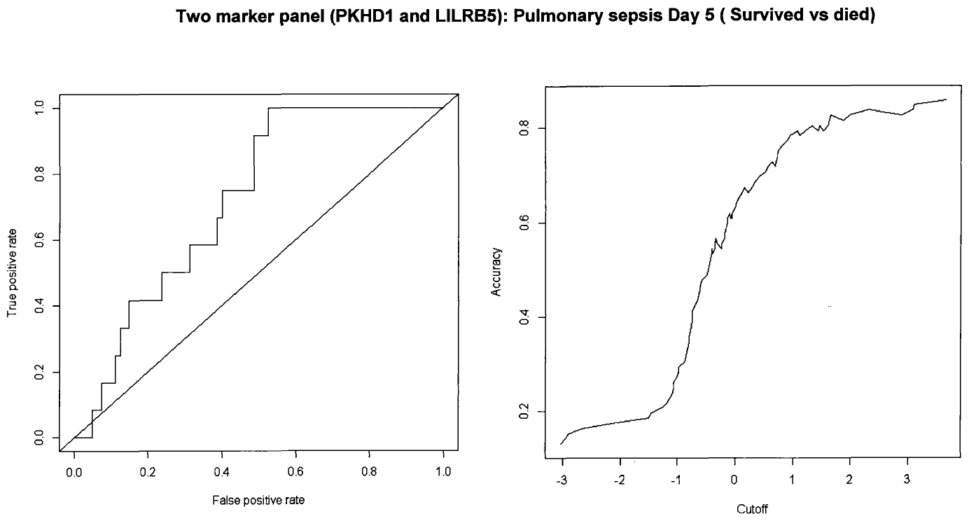

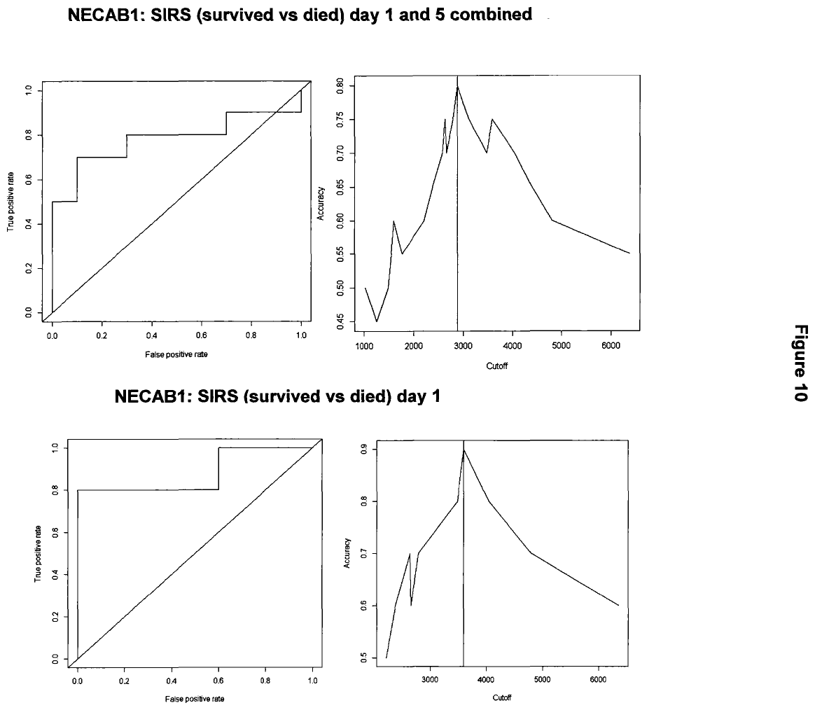

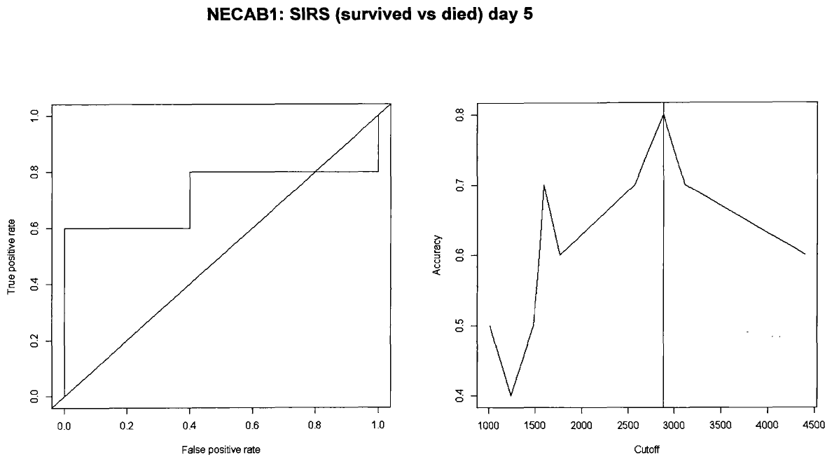

The protein quantification data obtained for the patients was analysed by ROC analysis to determine how well the markers could distinguish between different types of systemic inflammatory condition and/or between patients that survived and patients that died. The results of the analysis are presented in the table below. ROC Curves are shown in FIG. 10.

TABLE 13

| | Area under | Accuracy | |
|---|---|---|---|
| BIOMARKER/TEST | Curve (AUC) | (ACC) | Cut-off |
| DISC1: Pulmonary vs | 0.6935897 | 0.7959184 | ND |
| ALL Samples (day 0) | | | |
| DISC1: Pulmonary | 0.86 | 0.8333333 | 361.5500000 |
| vs controls | | | ng/ml |
| SLC39A8 (ZIP8): | 0.6538462 | 0.8723404 | 82.0000000 |
| Abdominal sepsis vs All | | | ng/ml |
| SLC39A8 (ZIP8): | 0.76875 | 0.8928571 | 57.2375000 |
| Abdominal vs Controls | | | ng/ml |
| GPR124: SIRS(OOHCA) | 0.6608187 | 0.7272727 | 1858.6600000 |
| vs all sepsis | | | pg/ml |
| GPR124: SIRS (OOHCA) | 0.6398892 | 0.6842105 | 1971.0400000 |
| VS Pulmonary sepsis | | | pg/ml |
| GPR124: SIRS (OHCA) | 0.6988304 | 0.7027027 | 2109.4800000 |
| vs Abdominal sepsis | | | pg/ml |
| NECAB1: SIRS: DIED | 0.78 | 0.80 | 2876.15 ng/ml |
| VS SURVIVED | | | |
| NECAB1: SIRS: DIED VS | 0.88 | 0.9 | 3591.25 ng/ml |
| SURVIVED (DAY 0) | | | |
| NECAB1: SIRS: DIED VS | 0.72 | 0.8 | 2876.15 ng/ml |
| SURVIVED (DAY 5) | | | |

The pulmonary sepsis marker DISC1 was tested for its ability to distinguish between patients having pulmonary sepsis and patients having another systemic inflammatory condition (such as abdominal sepsis or SIRS). DISC1 was also tested for its ability to distinguish between patients having pulmonary sepsis and healthy controls. ROC analysis revealed that this marker performed well to identify patients having pulmonary sepsis.

The abdominal sepsis biomarker SLC39A8 was tested for its ability to distinguish between patients having abdominal sepsis and patients having another systemic inflammatory condition (such as pulmonary sepsis or SIRS). DISC1 was also tested for its ability to distinguish between patients having abdominal sepsis and healthy controls. ROC analysis revealed that this marker performed well to identify patients having abdominal sepsis.

The SIRS biomarker GPR124 was tested for its ability to distinguish between patients having SIRS and patients having sepsis (including those having abdominal sepsis and pulmonary sepsis). ROC analysis revealed that this marker performed well to distinguish patients having SIRS and patients having any type of sepsis.

The survival biomarker NECAB1 was tested for its ability to distinguish between patients having SIRS that survived and patients having SIRS that died. ROC analysis revealed that this marker performed well as a survival marker for SIRS.

### EXAMPLE 4: DIAGNOSIS OF SYSTEMIC INFLAMMATORY DISEASE IN INTENSIVE CARE

A patient presents at an intensive care unit (ICU) or is admitted to lower dependency hospital ward with unspecified illness. Within 6 hours of admission, a blood sample is obtained from the patient, and is tested with inflammation (as described in Table 1), SIRS (as described in Table 2) and sepsis (as described in Table 3) biomarker panels using qPCR. Raised expression of inflammation markers, as determined by a lower threshold Cₜ value compared with controls, indicates an ongoing systemic inflammatory condition. Raised expression of SIRS biomarkers indicates that the systemic inflammatory condition is SIRS. Raised expression of sepsis biomarkers indicates that the systemic inflammatory condition is sepsis.

### EXAMPLE 5: DIAGNOSIS OF SYSTEMIC INFLAMMATORY DISEASE AT GP SURGERY, OUT-OF-HOURS CLINIC OR EMERGENCY DEPARTMENT

A patient presents at a GP surgery, an out-of-hours clinic, or an accident and emergency department. A blood sample is obtained from the patient and tested using a rapid point of care diagnostic test for inflammation markers (as described in Table 1). The test reveals that the inflammation biomarkers are elevated in the patient. The patient is referred for further detailed investigation using a full panel of inflammation, sepsis, and SIRS biomarkers in a hospital/diagnostic laboratory setting.

### EXAMPLE 6: MONITORING OF SYSTEMIC INFLAMMATORY CONDITION

A patient is undergoing treatment for a systemic inflammatory condition in an ICU or hospital. To monitor how the patient is responding to treatment, and whether continued treatment or a change in treatment is needed, a blood sample is taken and tested using a panel of prognostic recovery biomarkers (as described in Table 1).

If the level of prognosis biomarkers detected shows regression of the systemic inflammatory condition, treatment may be continued for a short while until the patient is deemed suitable for discharge. Prior to discharge, a blood sample from the patient may be taken and tested using a panel of survival biomarkers (as described in Table 4). If the levels of survival biomarkers show that the patient has a good prognosis of recovery, the patient is discharged. If the levels of survival biomarkers show that the patient has a poor prognosis of recovery, the patient is not discharged and treatment is continued.

If the levels of prognosis biomarkers show progression or no change in the systemic inflammatory condition, the patient continues to undergo treatment and may be switched to a different treatment strategy. The patient may be monitored by testing further blood sample(s) using the panel of prognosis biomarkers to determine whether the systemic inflammatory disease is progressing or regressing towards normalcy.

After discharge, the patient may be monitored at home by community nursing staff using a prognostic marker point of care diagnostic test, to monitor ongoing response to therapy and to provide rapid indication of any relapse.

### EXAMPLE 7: PREDICTION OF DISEASE SEVERITY AND SURVIVAL IN PATIENTS

A patient presents at an intensive care unit (ICU) or is admitted to lower dependency hospital ward with unspecified illness, and is diagnosed as having a systemic inflammatory condition. To assess the severity of the disease, a blood sample is taken and tested using a panel of prognostic biomarkers (as described in Table 1). The blood sample may also be tested using a panel of survival biomarkers (as described in Table 4) to determine the degree of organ damage/failure.

The levels of prognosis biomarkers and/or survival biomarkers may be used by clinicians to inform treatment choices/tailor treatment packages and manage survival expectation in the next of kin.

TABLE 14
Oligonucleotide probes

| Accession | Gene | | |
|---|---|---|---|
| No. | Symbol | Probes 1a & 1b | Probes 2a & 2b |
| NM_001124 | ADM | GTGTAAAGTTGTTCGCCGCGTGGA | CTGATTTCTCACGGCGTGTCACCCC |
| | | ATGTGAGTGTGTTTGTGTGCATGA | ACCAGGGCGCAAGCCTCACTATTAC |
| | | AAGAGAAAGACT | TTGAACTTTC |
| | | (SEQ ID NO: 86) | (SEQ ID NO: 88) |
| | | ATTTAGGCGCCCATGGTACAAGGA | TTACATAAAATGGGTGATATGCGAA |
| | | ATAGTCGCGCAAGCATCCCGCTGG | CAGCAAACCAATAAACTGTCTCAAT |
| | | TGCCTCCCGGGA | GCTGATTCAT |
| | | (SEQ ID NO: 87) | (SEQ ID NO: 89) |
| NM_020406 | CD177 | CTTGGACACCAGATTCTTTCCCAT | ATACTGCAGGCAATCTTAACACCAC |
| | | TCTGTCCATGAATCATCTTCCCCA | GGCAAGTATTTGTGCATCTACACAC |
| | | CACACAATCATT | ATCTAAACAT |
| | | (SEQ ID NO: 90) | (SEQ ID NO: 92) |
| | | GAGGAGAGGAGCCTAATGAGAAAA | GTAGCGTGCACTTACACCAACCCAG |
| | | TGACCATCTAAAGCCTGCCCTTCA | ATGGTACAGCCCAATACACACCCAG |
| | | TTGGTCTGGTTC | GATGGACGCT |
| | | (SEQ ID NO: 91) | (SEQ ID NO: 93) |
| NM_017565 | FAM20A | GTGGGAGGTCAATCCCCTTTACTG | TCCCAGCACTTTGGGCCTAAACAGG |
| | | TGACACAGTGAAACAGATCTACCC | CAGATCGCTTGGTCTCAGGAGCTCG |
| | | GTACAACAACAG | AGACCAGCCT |
| | | (SEQ ID NO: 94) | (SEQ ID NO: 96) |
| | | TCGTGCGTTGCCTTGCTCCGTTTT | CAAGGTCAGGATGGCATGGGAACAG |
| | | TCCCAAAAAGCACTGGCTTCATCA | GCCTAGCAGGGACACAAGCCTGGA |
| | | AGGCCACCGACG | GTAAGGCAGGA |
| | | (SEQ ID NO: 95) | (SEQ ID NO: 97) |
| NM_000572 | IL10 | CCTAACCTCATTCCCCAACCACTT | CCTGACCACGCTTTCTAGCTGTTGA |
| | | CATTCTTGAAAGCTGTGGCCAGCT | GCTGTTTTCCCTGACCTCCCTCTAA |
| | | TGTTATTTATAA | TTTATCTTGT |
| | | (SEQ ID NO: 98) | (SEQ ID NO: 100) |
| | | CATCAACTACATAGAAGCCTACAT | TGAGGCTACGGCGCTGTCATCGATT |
| | | GACAATGAAGATACGAAACTGAGA | TCTTCCCTGTGAAAACAAGAGCAAG |
| | | CATCAGGGTGGC | GCCGTGGAGC |
| | | (SEQ ID NO: 99) | (SEQ ID NO: 101) |
| NM_152637 | METTL7B | ATGGAAGCTGGGCCTTCATGTGGC | CTGCAAGTTTCTGGACTAGTCTCCC |
| | | AGCAAGTTTTCGAGCCCACCTGGA | AACGTTTGCCTCCCAATGTTGTCCC |
| | | AACACATTGGGG | TTTCCTTCGT |
| | | (SEQ ID NO: 102) | (SEQ ID NO: 104) |
| | | AGGCACTCATTTGCTCCTTCCCCA | CCCTCTCTCCCCAACCTCTGCCAGG |
| | | GCCTCCAATTAGAACAAGCCACCC | GCAATCTCTAACTTCAATCCCGCCT |
| | | ACCAGCCTATCT | TCGACAGTGA |
| | | (SEQ ID NO: 103) | (SEQ ID NO: 105) |
| NM_004994 | MMP9 | TGGAGGTGGGCTGGGCCCTCTCTT | GTTCGACGTGAAGGCGCAGATGGTG |
| | | CTCACCTTTGTTTTTTGTTGGAGT | GATCCCCGGAGCGCCAGCGAGGTG |
| | | GTTTCTAATAAA | GACCGGATGTT |
| | | (SEQ ID NO: 106) | (SEQ ID NO: 108) |
| | | GAGTTCCCGGAGTGAGTTGAACCA | CCTTCCTTATCGCCGACAAGTGGCC |
| | | GGTGGACCAAGTGGGCTACGTGAC | CGCGCTGCCCCGCAAGCTGGACTC |
| | | CTATGACATCCT | GGTCTTTGAGG |
| | | (SEQ ID NO: 107) | (SEQ ID NO: 109) |
| NM_020415 | RETN | CTCCAGGTCCGGAGGGGTTGCGGG | GAAGAAGCCATCAATGAGAGGATCC |
| | | GGAGCTGGAAATAAACCTGGAGA | AGGAGGTCGCCGGCTCCCTAATATT |
| | | TGATGATGATGAT | TAGGGCAATA |
| | | (SEQ ID NO: 110) | (SEQ ID NO: 112) |
| | | TCGTGGGATGTGCGCGCCGAGACC | GACCTGGCTACTTGCCCCCGAGGCT |
| | | ACATGTCACTGCCAGTGCGCGGGC | TCGCCGTCACCGGCTGCACTTGTG |
| | | ATGGACTGGACC | GCTCCGCCTGT |
| | | (SEQ ID NO: 111) | (SEQ ID NO: 113) |
| NM_153046 | TDRD9 | CTCTTTGGGTGATAGTCAGAGAGT | TACTGCCCGAGCACGACATGGAGCT |
| | | GGTGTTTTTGTTCAGGTGGGAAGG | TGCGTTTGACGTTCAATTCAGCGTG |
| | | ATTGGAAACTCT | GAGGATGTCG |
| | | (SEQ ID NO: 114) | (SEQ ID NO: 116) |
| | | GCTGCTATTAACAAGCTAGTCTGT | AGCCCTACGAGTGGAATCAGGTTGA |
| | | GATGGACCAAATGGATGCAAGTGT | TCCAAAGCTGGTCATGGAGCAGGCC |
| | | CTTGGGCCAGAG | GACCGTGAGA |
| | | (SEQ ID NO: 115) | (SEQ ID NO: 117) |
| NM_002206 | ITGA7 | AGGAGGTTGTGTCACTGACTCAGG | CTGTACTGGCTGGGCTGCTGGTGCT |
| | | CTGCTCCTTCTCTAGTTTCCCCTC | AGCACTGCTGGTGCTGCTCCTGTGG |
| | | TCATCTGACCTT | AAGATGGGAT |
| | | (SEQ ID NO: 118) | (SEQ ID NO: 120) |
| | | CAGAGATGGCTCCTTGGGATGAAG | GGTAGGGTGAGAAGGGCAGGGGTGT |
| | | AGGGTAGAGTGGGCTGCTGGTGTC | CCTGATGCAAAGGTGGGGAGAAGG |
| | | GCATCAAGATTT | GATCCTAATCC |
| | | (SEQ ID NO: 119) | (SEQ ID NO: 121) |
| NM_001721 | BMX | TTATGCTGCTCCTGATATAACACT | ACAGCAGCAAGTCAGACGTATGGGC |
| | | TTCCAGCCTATAGCAGAAGCACAT | ATTTGGGATCCTGATGTGGGAGGTG |
| | | TTTCAGACTGCA | TTCAGCCTGG |
| | | (SEQ ID NO: 122) | (SEQ ID NO: 124) |
| | | CCAGTATGTCAGTTCAGTCGGAAC | AGACAAGCATTGAAGAAGAAATTAG |
| | | AAAGTTTCCAGTCAAGTGGTCAGC | GAGTGCTGATAAGAATGAATATAGA |
| | | TCCAGAGGTGTT | TGCTGGCCAG |
| | | (SEQ ID NO: 123) | (SEQ ID NO: 125) |
| NM_005143 | HP | GATAAGATGTGGTTTGAAGCTGAT | GCAGTGCCTTTGCCGTTCACGACCT |
| | | GGGTGCCAGCCCTGCATTGCTGAG | GGAGGAGGACACCTGGTATGCGACT |
| | | TCAATCAATAAA | GGGATCTTAA |
| | | (SEQ ID NO: 126) | (SEQ ID NO: 128) |
| | | GAAGACCATAGCTGAGAACTAATG | GGCGAAATGCCAATTTTAAATTTAC |
| | | CAAGGCTGGCCGGAAGCCCTTGCC | TGACCATCTGAAGTATGTCATGCTG |
| | | TGAAAGCAAGAT | CCTGTGGCTG |
| | | (SEQ ID NO: 127) | (SEQ ID NO: 129) |
| NM_000597 | IGFBP2 | GGAGGGGGAAGAGAAATTTTTATT | CCAACTGTGACAAGCATGGCCTGTA |
| | | TTTGAACCCCTGTGTCCCTTTTGC | CAACCTCAAACAGTGCAAGATGTCT |
| | | ATAAGATTAAAG | CTGAACGGGC |
| | | (SEQ ID NO: 130) | (SEQ ID NO: 132) |
| | | TTCCAGTTCTGACACACGTATTTA | CCTCAAGTCGGGTATGAAGGAGCTG |
| | | TATTTGGAAAGAGACCAGCACCGA | GCCGTGTTCCGGGAGAAGGTCACT |
| | | GCTCGGCACCTC | GAGCAGCACCG |
| | | (SEQ ID NO: 131) | (SEQ ID NO: 133) |
| NM_000478 | ALPL | GCTACAAGGTGGTGGGCGGTGAAC | TTCTGGATCTGACCCTCCCAGTCTC |
| | | GAGAGAATGTCTCCATGGTGGAC | ATCTCCTGACCCTCCCACTCCCATC |
| | | TATGCTCACAACA | TCCTTACCTC |
| | | (SEQ ID NO: 134) | (SEQ ID NO: 136) |
| | | TGGGCTCTGAACACACACGCCAGC | CCTCAGCCTCTGCAACTGCAAGAAA |
| | | TCCTCTCTGAAGCGACTCTCCTGT | GGGGACCCAAGAAACCAAAGTCTGC |
| | | TTGGAACGGCAA | CGCCCACCTC |
| | | (SEQ ID NO: 135) | (SEQ ID NO: 137) |
| NM_005143 | DACH1 | GAACAAGCAGAACAGACGCTAAAA | CATTATAGATACTCTGGCATTACGC |
| | | CAGGCAGCTTCAACAGATAGTCT | TTCTATACCTTTTAGGTCTTCCTTG |
| | | CAGGGTCTTAAAT | CAATACTGGA |
| | | (SEQ ID NO: 138) | (SEQ ID NO: 140) |
| | | AATATTAATGTCTAGTTGTTCTAT | CTTGATGTACCAGTCCAATACCATG |
| | | ATTATAACCACATTTGCGCTCTAT | TAGCGCTGAGTGATAAAGTTAAAAT |
| | | GCAAGCCCTTGG | GTGCTGTGCT |
| | | (SEQ ID NO: 139) | (SEQ ID NO: 141) |
| NM_000877 | IL1R1 | ATAATTTTCCTCCTAAACAAAAAC | AAAGCCAAATTTATATGCCACCGAT |
| | | ACATTGAGTTTAAGTCTCTGACTC | TGCAGGACACAAGCACAGTTTTAAG |
| | | TTGCCTTTCCAC | AGTTGTATGA |
| | | (SEQ ID NO: 142) | (SEQ ID NO: 144) |
| | | TGGAAAAACAGTGTGGATATAAGC | ACCATCCTTCCCATGATGCCGCTCT |
| | | TGTTCATTTATGGAAGGGATGACT | TCTGTCATCCCGCTCCTGCTGAAAC |
| | | ACGTTGGGGAAG | ACCTCCCAGG |
| | | (SEQ ID NO: 143) | (SEQ ID NO: 145) |
| NM_00103970 | OLAH | GGATCTGAAGACATAGCAAAGGAC | GTAGTCCCATCATAAGGGCAGATCT |
| 2 | | ATGGAAGCCTGGAAAGATGTAAC | GAACATTGTTAGAAGTTGCACCTCT |
| | | CAGTGGAAATGCT | AACGTACCAT |
| | | (SEQ ID NO: 146) | (SEQ ID NO: 148) |
| | | GCTGAGGCAGGAGAATGGTGTGAA | AATTACACATTTTCTACTGTCAGGG |
| | | CCTGGGAGGTGGAGCTTGCAGTGA | AGATTCGTTACATAAATATATTTAC |
| | | ACCGAGATCGCT | GTATCTGGGG |
| | | (SEQ ID NO: 147) | (SEQ ID NO: 149) |
| NM_004633 | IL1R2 | TGGTACAAGGATTCTCTTCTTTTG | CATTTGCCCATGAAGGCCAGCAATA |
| | | GATAAAGACAATGAGAAATTTCTA | CAACATCACTAGGAGTATTGAGCTA |
| | | AGTGTGAGGGGG | CGCATCAAGA |
| | | (SEQ ID NO: 150) | (SEQ ID NO: 152) |
| | | TCAAGGAAGCCTCCTCCACGTTCT | CCGAGGGGCCACGCCAGGAATATTC |
| | | CCTGGGGCATTGTGCTGGCCCCAC | AGAAAATAATGAGAACTACATTGAA |
| | | TTTCACTGGCCT | GTGCCATTGA |
| | | (SEQ ID NO: 151) | (SEQ ID NO: 153) |
| NM_031226 | CYP19A1 | GACATTGATTTGCTCTTACTACAG | ATTCGGCAGCAAACTTGGGCTGCAG |
| | | CTTCAGTGATTGGGGGAGGAAAAG | TGCATCGGTATGCATGAGAAAGGCA |
| | | TCCCAACCCAAT | TCATATTTAA |
| | | (SEQ ID NO: 154) | (SEQ ID NO: 156) |
| | | GACTGAATCTCTCACCTATTCTTG | GCTCAGAGATACTCCCAACTGATGC |
| | | CAGAAAGACATACTAATTAAACCT | AGAAACCAAATAAAGAGGTAGGTAT |
| | | TGTCAAAGTAGT | TCCAAGAATT |
| | | (SEQ ID NO: 155) | (SEQ ID NO: 157) |
| NM_002424 | MMP8 | ATCTGACTTCTAGGATTTATTGTT | GAGGCTTATTCAGTTCTTACACATT |
| | | ATATTACTTGCCTATCTGACTTCA | CCATCTTACATTAGTGATTCCATCA |
| | | TACATCCCTCAG | AAGAGAAGGA |
| | | (SEQ ID NO: 158) | (SEQ ID NO: 160) |
| | | GGCATAGTCACCTAGGGGAGGAGG | GATGGGTTTTTTTGTTAAGAACTAT |
| | | CCGTATGAAGACAGAGGCAGAGAT | AGGATTTATGGGACCAAGTCTAGCG |
| | | TGGAGTGACGCA | AGTCCAGATA |
| | | (SEQ ID NO: 159) | (SEQ ID NO: 161) |
| NM_003236 | TGFα | TTTTTTAAGCATCCTGACAGGAAA | GCCCAGTCACAGAAGGAGGAATGAC |
| | | TGTTTTCTTCTACATGGAAAGATA | TCAAATGCCCAAAACCAAGAACACA |
| | | GACAGCAGCCAA | TTGCAGAAGT |
| | | (SEQ ID NO: 162) | (SEQ ID NO: 164) |
| | | TTAGAACTCCTTACTCTGATGTCT | ATGAGGCTCTAACACTGCTCAGGAG |
| | | GTATATGTTGCACTGAAAAGGTTA | ACCCCTGCCCTCTAGTTGGTTCTGG |
| | | ATATTTAATGTT | GCTTTGATCT |
| | | (SEQ ID NO: 163) | (SEQ ID NO: 165) |
| NM_198481 | VSTM1 | TGGCCAAGGTTATCGGAAATCTGG | ATGAAAACAGACACCAGAACCATCT |
| | | AGATGCAGATACTGTGTTTCCTTG | TTGTCGCCATCTTCAGCTGCATCTC |
| | | CTCTTCGTCCAT | CATCCTTCTC |
| | | (SEQ ID NO: 166) | (SEQ ID NO: 168) |
| | | CCCAGGAGCCCCCAGGATCTCATG | AGGTGAACGACTCTGGGTACAAGCA |
| | | AATATGCGGCACTGAAAGTGTAGC | GGAACAGAGCTCGGCAGAAAACGA |
| | | AAGAAGACAGCC | AGCTGAATTCC |
| | | (SEQ ID NO: 167) | (SEQ ID NO: 169) |
| NM_002001 | FCER1A | AGCTCCGCGTGAGAAGTACTGGCT | GTCAGTTCCACCAAATGGTTCCACA |
| | | ACAATTTTTTATCCCATTGTTGGT | ATGGCAGCCTTTCAGAAGAGACAAA |
| | | GGTGATTCTGTT | TTCAAGTTTG |
| | | (SEQ ID NO: 170) | (SEQ ID NO: 172) |
| | | GCCATGGTTGGAGGAACTGGGATG | AGTGGCATGTAATAGTAAGTGCTCA |
| | | TGTACAAGGTGATCTATTATAAGG | ATTAACATTGGTTGAATAAATGAGA |
| | | TAGGTGAAGCTC | GAATGAATAG |
| | | (SEQ ID NO: 171) | (SEQ ID NO: 173) |
| NM_007360 | KLRK1 | CCCATGTCCTAAAAACTGGATATG | TGTTTGTCCCACTATTGTATTTTGG |
| | | TTACAAAAATAACTGCTACCAATT | AAGCACATAACTTGTTTGGTTTCAC |
| | | TTTTGATGAGAG | AGGTTCACAG |
| | | (SEQ ID NO: 174) | (SEQ ID NO: 176) |
| | | ATCTCAATAAAAGCCAGGAACAGA | GCTGTTTTAATTTCTAAAGGTAGGA |
| | | GAAGAGATTACACCAGCGGTAACA | CCAACACCCAGGGGATCAGTGAAGG |
| | | CTGCCAACTGAG | AAGAGAAGGC |
| | | (SEQ ID NO: 175) | (SEQ ID NO: 177) |
| NM_002258 | KLRB1 | TCAACCCTTGGAATAACAGTCTAG | CCCTGAAACTTAGCTGTGCTGGGAT |
| | | CTGATTGTTCCACCAAAGAATCCA | TATTCTCCTTGTCTTGGTTGTTACT |
| | | GCCTGCTGCTTA | GGGTTGAGTG |
| | | (SEQ ID NO: 178) | (SEQ ID NO: 180) |
| | | CAGAAATCATCAATAGAAAAATGC | AGATGGATCTGCCAAAAAGAACTAA |
| | | AGTGTGGACATTCAACAGAGCAGG | CACCTGTGAGAAATAAAGTGTATCC |
| | | AATAAAACAACA | TGACTCTTGA |
| | | (SEQ ID NO: 179) | (SEQ ID NO: 181) |
| NM_015345 | DAAM2 | GTCATCAACCTAACAAACACAACC | GAACTGTGACTATCTATCTCCCCCG |
| | transcript | TTCTCAGCAGCATTTCTCCCCTGT | ACTTCTACCAGGGATGCCTTCACGC |
| | variants | GATGGAAATAAA | CAAGGCTGTT |
| | 1 & 2 | (SEQ ID NO: 182) | (SEQ ID NO: 184) |
| | | CTGGTGCCCAGTCGGGGTGGCTGA | TGGTTTGAGGGGGCGGGCGGTGTGT |
| | | GCTGGTCCTTAATAGGTTGTTTCT | GTGTGTTCTGGTGGGAGGGATCTG |
| | | TGGTCTTGCTTT | AGCAAGTGCAA |
| | | (SEQ ID NO: 183) | (SEQ ID NO: 185) |
| NM_019111 | HLA-DRA | AGAAGATCACTGAAGAAACTTCTG | GGATATGCCTCTTCGATTGCTCCGT |
| | | CTTTAATGGCTTTACAAAGCTGGC | ACTCTAACATCTAGCTGGCTTCCCT |
| | | AATATTACAATC | GTCTATTGCC |
| | | (SEQ ID NO: 186) | (SEQ ID NO: 188) |
| | | CCTCTGGAATAAAACATACAGGAG | CGTTTACGACTGCAGGGTGGAGCAC |
| | | TCTGTCTCTGCTATGGAATGCCCC | TGGGGCTTGGATGAGCCTCTTCTCA |
| | | ATGGGGCATCTC | AGCACTGGGA |
| | | (SEQ ID NO: 187) | (SEQ ID NO: 189) |
| NM_138576 | BCL11B | AAACCCGTGATTTTGGTGCTCCTT | ATTGGAACCTGCCACTTGGCATTAG |
| | | GTAACTCAGCCCTGCAAAGCAAAG | AGGGTCTTTCATGGGGAGAGAAGGA |
| | | TCCCATTGATTT | GACTGAATTA |
| | | (SEQ ID NO: 190) | (SEQ ID NO: 192) |
| | | CTCCAACCTAACCTGTGTCTGCGA | GCCTGGGATGAATTTGGTGCCTTTC |
| | | AGTCCTATGGAAACCCGAGGGTTG | CATATCTCGTTCTCTCTCCTTCCCC |
| | | ATTAAGGCAGTA | TGCGTTTCCT |
| | | (SEQ ID NO: 191) | (SEQ ID NO: 193) |
| NM_004867 | ITM2A | CTAGTTGCTGTGGAGGAAATTCGT | AATGACTGCTTACCTGGACTTGTTG |
| | | GATGTTAGTAACCTTGGCATCTTT | CTGGGGAACTGCTATCTGATGCCCC |
| | | ATTTACCAACTT | TCAATACTTC |
| | | (SEQ ID NO: 194) | (SEQ ID NO: 196) |
| | | ATTAAGGTTTATGGGATACTCAAG | TGTTGGTGGAGCCTGCATTTACAAG |
| | | ATATTTACTCATGCATTTACTCTA | TACTTCATGCCCAAGAGCACCATTT |
| | | TTGCTTATGCTT | ACCGTGGAGA |
| | | (SEQ ID NO: 195) | (SEQ ID NO: 197) |
| NM_052931 | SLAMF6 | TGCCTAACCTTTTGGAGCCTTAGT | AGCATTACCCTTCTGACACTCTCTA |
| | | CTCCCAGACTGAAAAAGGAAGAGG | TGTAGCCTCCCTGATCTTCTTTCAG |
| | | ATGGTATTACAT | CTCCTCTATT |
| | | (SEQ ID NO: 198) | (SEQ ID NO: 200) |
| | | TAAAATCCCAGCTACTTGAGAGAC | TCAGAAATATTTCTTGGACCTTCCA |
| | | TGAGGCAGGAGAATCGCTTGAACC | CTTCTCCTCCAACTCCTTGACCACC |
| | | CAGGAGGTGGAG | ATCCTGTATC |
| | | (SEQ ID NO: 199) | (SEQ ID NO: 201) |
| NM_002121 | HLA-DPB1 | CTGGATAGTCCTGTCACCGTGGAG | CATTTGCTGTGTTTCGTTAGCATCT |
| | | TGGAAGGCACAGTCTGATTCTGCC | GGCTCCAGGACAGACCTTCAACTTC |
| | | CGGAGTAAGACA | CAAATTGGAT |
| | | (SEQ ID NO: 202) | (SEQ ID NO: 204) |
| | | TGCTTGTCTGCCACGTGACGGATT | TAATGGGACACAGCGCTTCCTGGAG |
| | | TCTACCCAGGCAGCATTCAAGTCC | AGATACATCTACAACCGGGAGGAGC |
| | | GATGGTTCCTGA | TCGTGCGCTT |
| | | (SEQ ID NO: 203) | (SEQ ID NO: 205) |
| NM_007053 | CD160 | TACCAAGAATCTGTGGAAATATAA | TCCTCCCTCTTCATCAACATAGTAA |
| | | GCTGGGGCAAATCAGTGTAATCCT | AATAAGTCAAACAAAATGAGAACAC |
| | | TGACTTTGCTCC | CAAATTTTGG |
| | | (SEQ ID NO: 206) | (SEQ ID NO: 208) |
| | | ATGCAGACAGACCTCAACATTCAA | AAACAAGCAAAGATAGGTAGGACAG |
| | | CAACATCCATACAGCACTGCTGGA | AAAGGAAGACAGCCAGATCCAGTGA |
| | | GGAAGAGGAAGA | TTGACTTGGC |
| | | (SEQ ID NO: 207) | (SEQ ID NO: 209) |
| NM_016523 | KLRF1 | TACGTGATAGTATAAACCAATGTG | TTCCAGGCTTTTGCTACTCTTCACT |
| | | ACTTCATGTGATCATATCCAGGAT | CAGCTACAATAAACATCCTGAATGT |
| | | TTTTATTCGTCG | TTTCTTAAAA |
| | | (SEQ ID NO: 210) | (SEQ ID NO: 212) |
| | | ATCTAAAAGTGAATAATGGCACAA | CAAATACCAAGGGAAGTGTTATTGG |
| | | GAAGAAATATAAGTAATAAGG | TTCTCTAATGAGATGAAAAGCTGGA |
| | | ACCTTTGTGCTTCGA | GTGACAGTTA |
| | | (SEQ ID NO: 211) | (SEQ ID NO: 213) |
| NM_001767 | CD2 | GAGTTTCTTATGTGCCCTGGTGGA | CTCTGAAAATTAAGCATCTGAAGAC |
| | | CACTTGCCCACCATCCTGTGAGTA | CGATGATCAGGATATCTACAAGGTA |
| | | AAAGTGAAATAA | TCAATATATG |
| | | (SEQ ID NO: 214) | (SEQ ID NO: 216) |
| | | GACACCGTGTTCAGCACCAGCCTC | ACACAACCCTGACCTGTGAGGTAAT |
| | | AGAAGAGGCCTCCTGCTCCGTCGG | GAATGGAACTGACCCCGAATTAAAC |
| | | GCACACAAGTTC | CTGTATCAAG |
| | | (SEQ ID NO: 215) | (SEQ ID NO: 217) |
| NM_006498 | LGALS2 | CTGAGCTACCTGAGCGTAAGGGGC | GAGAGTGACAAATTCAAGGTGAAGC |
| | | GGGTTCAACATGTCCTCTTTCAAG | TGCCAGATGGGCACGAGCTGACTTT |
| | | TTAAAAGAATAA | TCCCAACAGG |
| | | (SEQ ID NO: 218) | (SEQ ID NO: 220) |
| | | CCTGCATTTCAACCCTCGCTTCAG | CAGGCAGCATCGCCGATGGCACTGA |
| | | CGAATCCACCATTGTCTGCAACTC | TGGCTTTGTAATTAATCTGGGCCAG |
| | | ATTGGACGGCAG | GGGACAGACA |
| | | (SEQ ID NO: 219) | (SEQ ID NO: 221) |
| NM_024409 | NPPC | TGCAAGAGCACCCCAACGCGCGCA | ATCGGGAACTGGCTCCGTTGTGCTG |
| | | AATACAAAGGAGCCAACAAGAAG | AGGTCATCTTTGGTCATCAGCCTCC |
| | | GGCTTGTCCAAGG | AGCATCTGGA |
| | | (SEQ ID NO: 222) | (SEQ ID NO: 224) |
| | | CTCAAGCTGGACCGAATCGGCTCC | CGGGGGCGCCAATCTCAAGGGCGAC |
| | | ATGAGCGGCCTGGGATGTTAGTGC | CGGTCGCGACTGCTCCGGGACCTG |
| | | GGCGCCCCCTGG | CGCGTGGACAC |
| | | (SEQ ID NO: 223) | (SEQ ID NO: 225) |
| NM_005376 | MYCL | TGGGAGCCAGCCTCCCTTTGATGA | CCTAGGGGGAGAAGAAGCCGAGAGC |
| | Transcript | TTATTGGAGCCCCAGGGGACAAGG | CTTTTGTGCAAAGCCAAAACCTTCG |
| | variant 3 | GATTTGAGGTGA | TCCTTTTAAA |
| | | (SEQ ID NO: 226) | (SEQ ID NO: 228) |
| | | GTGTCCTTCTTGCTCCCCCTCAAT | GGTCCACTGTGTTAAGGTCATTTTT |
| | | AGATCTCCAGCGTCAGCTGCTCCC | AACCAGCTTGCTTTCTACACCAAGA |
| | | TGGCATTCAACA | GTTTATGTTT |
| | | (SEQ ID NO: 227) | (SEQ ID NO: 229) |
| NM_00103308 | MYCL | GTATTTAGTTGTGATTACTGATTG | ACTCGGCAGCTCCAAGTGGAATCCA |
| 1 | Transcript | CCTGATTTTAAAATGTTGCCTTCT | CGTGCAGCTTCTAGTCTGGGAAAGT |
| | variant 1 | GGGACATCTTCT | CACCCAACCT |
| | | (SEQ ID NO: 230) | (SEQ ID NO: 232) |
| | | CTTCTGGAGCATGGTTTACAAAAG | CATAATGGTTTCTTTCTGAGGTTGC |
| | | CCAGCTGACTTCTGGAATTGTCTA | TTCTTGGCCTCAGAGGACCCCAGGG |
| | | TGGAGGACAGTT | GATGTTTGGA |
| | | (SEQ ID NO: 231) | (SEQ ID NO: 233) |
| NM_0011449 | MX1 | CAGCTTATTTCCTCATTTTTATAA | TAGCGAGGAGGTGCTGAAGAGCGCA |
| 25.2 | | TGTCCCTTCACAAACCCAGTGTTT | GGTTTGGAGAATGATCACCTGGATT |
| | | TAGGAGCATGAG | GGAACCATAG |
| | | (SEQ ID NO: 234) | (SEQ ID NO: 236) |
| | | AACCTCCACAGAACCGCCAAGTCC | AGCCTGCTGACATTGGGTATAAGAT |
| | | AAAATTGAAGACATTAGAGCAGAA | CAAGACACTCATCAAGAAGTACATC |
| | | CAAGAGAGAGAA | CAGAGGCAGG |
| | | (SEQ ID NO: 235) | (SEQ ID NO: 237) |
| NM_002985 | CCL5 | AGATGAGCTAGGATGGAGAGTCCT | TATCCTACCCCACCCGCTCCTTGAA |
| | | TGAACCTGAACTTACACAAATTTG | GGGCCCAGATTCTACCACACAGCAG |
| | | CCTGTTTCTGCT | CAGTTACAAA |
| | | (SEQ ID NO: 238) | (SEQ ID NO: 240) |
| | | AGGAGCTTACTGGCAAACATGAAA | GGTGACAAAGTGAGACTCCGTCACA |
| | | AATCGGCTTACCATTAAAGTTCTC | ACAACAACAACAAAAAGCTTCCCCA |
| | | AATGCAACCATA | ACTAAAGCCT |
| | | (SEQ ID NO: 239) | (SEQ ID NO: 241) |
| NM_000358 | TGFBI | AGCTATGAGTTGAAATGTTCTGTC | CACTACAGGAGGAATGCACCACGGC |
| | | AAATGTGTCTCACATCTACACGTG | AGCTCTCCGCCAATTTCTCTCAGAT |
| | | GCTTGGAGGCTT | TTCCACAGAG |
| | | (SEQ ID NO: 242) | (SEQ ID NO: 244) |
| | | TCAAGCAATCCAGCCTCATGGGAA | AAGAAATGGTATGTAGAGCTTAGAT |
| | | GTCCTGGCACAGTTTTTGTAAAGC | TTCCCTATTGTGACAGAGCCATGGT |
| | | CCTTGCACAGCT | GTGTTTGTAA |
| | | (SEQ ID NO: 243) | (SEQ ID NO: 245) |
| NM_005084 | PLA2G7 | AAAGCATTTAGGACTTCATAAAGA | AATGCTACTCACCTGATAAAGAAAG |
| | | TTTTGATCAGTGGGACTGCTTGAT | AAAGATGATTACAATCAGGGGTTCA |
| | | TGAAGGAGATGA | GTCCACCAGA |
| | | (SEQ ID NO: 246) | (SEQ ID NO: 248) |
| | | ATGTAGGCTATACTGTAATCGTGA | AAATAGCAGTAATTGGACATTCTTT |
| | | TTGAAGCTTGGACTAAGAATTTTT | TGGTGGAGCAACGGTTATTCAGACT |
| | | TCCCTTTAGATG | CTTAGTGAAG |
| | | (SEQ ID NO: 247) | (SEQ ID NO: 249) |
| NM_018125 | ARHGEF1 | ATAAAGTCTGTACATATTGGAGCT | GGACTTGTGGATGGGCCTGGACTCT |
| | 0L | CTGGGAGATGCTGGAATAAAAGAC | CCAGAAACTACTTGGGCAGAGCAAA |
| | | AAGAGTTACATC | GGAAAACCTC |
| | | (SEQ ID NO: 250) | (SEQ ID NO: 252) |
| | | CATCTGGAGGAAATGGCCTTCTTT | GATAGCCACGTGGGCCGAGAGCTGA |
| | | TTAAAAGCAAAAAACACAAAACCT | CCCGCAAGAAGGGCATCCTCTTGC |
| | | CACAACTGCCTG | AGTACCGCCTG |
| | | (SEQ ID NO: 251) | (SEQ ID NO: 253) |
| NM_032777 | ADGRA2/ | AAAGCTTTGTATTATTCTTCCACA | GTGAACTGAGGGGAGTAGAGGGAGA |
| | GPR124 | TATGCTGGCTGCTGTTTACACACC | GGGCAGGTGGAACTGGGGCAGAAT |
| | | CTGCCAATGCCT | CTAGTCATGCC |
| | | (SEQ ID NO: 254) | (SEQ ID NO: 256) |
| | | AGTAGAGAGAAACCTACAAAATGT | GGAAAACCCACACACACTCCTTGGA |
| | | CAAACCAGCTTCCCGACTCCCAGG | ATGGGTCCTGTTATTTATGCTTGCT |
| | | AGCTCAAGCCAA | GCACAGACAT |
| | | (SEQ ID NO: 255) | (SEQ ID NO: 257) |
| NM_173843 | IL1RN | CTCCAGAATGGTCTTTCTAATGTG | CAGCCTCACCAATATGCCTGACGAA |
| | | TGAATCAGAGCACAGCAGCCCCTG | GGCGTCATGGTCACCAAATTCTACT |
| | | CACAAAGCCCTT | TCCAGGAGGA |
| | | (SEQ ID NO: 258) | (SEQ ID NO: 260) |
| | | TGCAAAGTTCCCTACTTCCTGTGA | TTGCTCCTTGACATTGTAGAGCTTC |
| | | CTTCAGCTCTGTTTTACAATAAAA | TGGCACTTGGAGACTTGTATGAAAG |
| | | TCTTGAAAATGC | ATGGCTGTGCCTCTGCCTGT |
| | | (SEQ ID NO: 259) | (SEQ ID NO: 261) |
| NM_004895 | NLRP3 | TTAGAAACACTTCAAGAAGAAAAG | CGACACCTTGATATGGTGCAGTGTG |
| 4 | | CCTGAGCTGACCGTCGTCTTTGAG | TCCTCCCAAGCTCCTCTCATGCTGC |
| | | CCTTCTTGGTAG | CTGTTCTCAT |
| | | (SEQ ID NO: 262) | (SEQ ID NO: 264) |
| NM_0010798 | | AGCATCGGGTGTTGTTGTCATCAC | GGAGTCCGACCTCAGGAATCATGGA |
| 21 | | AGCGCCTCAGTTAGAGGATGTTCC | CTGCAGAAGGCGGATGTGTCTGCTT |
| | | TCTTGGTGACCTCATGTAATTA | TCCTGAGGAT |
| | | (SEQ ID NO: 263) | (SEQ ID NO: 265) |
| NM_0013235 | RBP4 | TCAGTTCCCATAAAACCTTCATTA | TGCAGTACTCCTGCCGCCTCCTGAA |
| 17.1 | | CACATAAAGATACACGTGGGGGTC | CCTCGATGGCACCTGTGCTGACAGC |
| | | AGTGAATCTGCT | TACTCCTTCG |
| | | (SEQ ID NO: 266) | (SEQ ID NO: 268) |
| | | GTGCCTGGCCAGGCAGTACAGGCT | GTGCGCAGACATGGTGGGCACCTTC |
| | | GATCGTCCACAACGGTTACTGCGA | ACAGACACCGAGGACCCTGCCAAG |
| | | TGGCAGATCAGA | TTCAAGATGAA |
| | | (SEQ ID NO: 267) | (SEQ ID NO: 269) |
| NM_001932 | MPP3 | TAAGTGGGAAGTCTTGTTTGTTGT | GACTGTGAGGGCTACCTCAAAGGGC |
| | | TGGTTTTTGTCTGTTGTTTTTCAC | ACTATGTGGCTGGTCTTCGGAGGAG |
| | | TGCACCTCTTTG | CTTCCGGCTG |
| | | (SEQ ID NO: 270) | (SEQ ID NO: 272) |
| | | AGCCATGAGAAGGAAGGAGTGGAA | GCCTGACAATATCGATGAGGATTTT |
| | | TATCACTTTGTGTCTAAGCAAGCA | GATGAGGAATCGGTGAAGATCGTCC |
| | | TTTGAGGCCGAC | GCTTGGTGAA |
| | | (SEQ ID NO: 271) | (SEQ ID NO: 273) |
| NM_006845 | KIF2C | AGAGGAAGGAGCTCTTAGTTACCC | GGAGAGCAGTTGATTCAAATGGAAA |
| | | TTTTGTGTTGCCCTTCTTTCCATC | CAGAAGAGATGGAAGCCTGCTCTAA |
| | | AAGGGGAA | CGGGGCGCTG |
| | | (SEQ ID NO: 274) | (SEQ ID NO: 276) |
| | | CTGGAGACCTTTGTGAACAAAGCG | GTATCTGGAGAACCAAGCATTCTGC |
| | | GAATCTGCTCTGGCCCAGCAAGCC | TTTGACTTTGCATTTGATGAAACAG |
| | | AAGCATTTCTCA | CTTCGAATGA |
| | | (SEQ ID NO: 275) | (SEQ ID NO: 277) |
| NM_002373 | MAP1A | AATGCCAGAATTCTTCCAAACTCC | CACGCTTCCCTGCTATAGTTCCCAG |
| | | CTGACTCTTTGAAGTTTTTACTCA | CTGCTGTAACGGAGCCACCTCCAAC |
| | | CCCCATTTCAAT | TCTAACAATA |
| | | (SEQ ID NO: 278) | (SEQ ID NO: 280) |
| | | GCTTTATACCATTCACATCCCAGG | CCCCTGCTTGGCTTCTCTGCATGTG |
| | | GCTGTGTCCAGACAGCACAAAACG | GTCATCTGCTGTGGCTTGGTGTTTA |
| | | GCAAGGAGAGCC | ATGGGTTAAA |
| | | (SEQ ID NO: 279) | (SEQ ID NO: 281) |
| NM_003005 | SELP | ATAGGTCTGATAATGGGTGGGACG | GTCAGCTACTCCACCAACCTGCAAA |
| | | CTCCTGGCTTTGCTAAGAAAGCGT | GGCATAGCATCACTTCCTACTCCAG |
| | | TTCAGACAAAAA | GGGTGCAATG |
| | | (SEQ ID NO: 282) | (SEQ ID NO: 284) |
| | | AAATACCTCTTTATTTTTTGATTG | AATTCTCAACCTACCACCCCTTCCT |
| | | AAGGAAGGTTTTCTCCACTTTGTT | GTCCCACCTCTTCTCTTCCTGTAAC |
| | | GGAAAGCAGGTG | ACAAGCCACA |
| | | (SEQ ID NO: 283) | (SEQ ID NO: 285) |
| NM_144573 | NEXN | TGAGCAGGATATGTTAGAAAAGAG | AGAGAAGATGAAAAAAGGAAAGCAG |
| | | GAAAATACAGCGTGAATTAGCAAA | AAGAAGAAGCCAGAAGGAGAATAGA |
| | | AAGGG | GGAAGAAAAG |
| | | (SEQ ID NO: 286) | (SEQ ID NO: 288) |
| | | GCGGGGCCAAAAAAGGAAACCAGG | GAAGGTAGCATCATGAATGGCTCCA |
| | | AGTGCCACTATGCTGACTTCTTAT | CTGCTGAAGATGAAGAGCAAACCAG |
| | | TCCTTTT | ATCAGGAGCT |
| | | (SEQ ID NO: 287) | (SEQ ID NO: 289) |
| NM_000419 | ITGA2B | ACAACAATGGCCCTGGGACTGTGA | GTGAGCTGGGCAACCCCATGAAGAA |
| | | ATGGTCTTCACCTCAGCATCCACC | GAACGCCCAGATAGGAATCGCGAT |
| | | TTCCGGGACAGT | GTTGGTGAGCG |
| | | (SEQ ID NO: 290) | (SEQ ID NO: 292) |
| | | TCGTAAGCTGCGACTCGGCGCCCT | GGGCCTTGGAGGAGAGGGCCATTCC |
| | | GTACTGTGGTGCAGTGTGACCTGC | AATCTGGTGGGTGCTGGTGGGTGT |
| | | AGGAGATGGCGC | GCTGGGTGGCC |
| | | (SEQ ID NO: 291) | (SEQ ID NO: 293) |
| NM_181526 | MYL9 | TGAGGAAGTGGACGAGATGTACCG | CTTCACGTGTATCCCCACACAAATG |
| | | GGAGGCACCCATTGATAAGAAAG | CAAGCTCACCAAGGTCCCCTCTCAG |
| | | GCAACTTCAACTA | TCCCCTTCCC |
| | | (SEQ ID NO: 294) | (SEQ ID NO: 296) |
| | | CCTCCCCGCACACACCCGTCCATA | TCAAACATGGCGCCAAGGATAAAGA |
| | | CCAGCTCCCTGCCCATGACCCTCG | CGACTAGGCCACCCCAGCCCCCTGA |
| | | CTCAGGGATCCC | CACCCCAGCC |
| | | (SEQ ID NO: 295) | (SEQ ID NO: 297) |
| NM_0002122 | ITGB3 | ATGTGTGGACACATTGGACCTTTC | TCCCATGAGTTGGCTGGGAATAAGT |
| | | CTGAGGAAGAGGGACTGTTCTTTT | GCCAGGATGGAATGATGGGTCAGTT |
| | | GTCCCAGAAAAG | GTATCAGCAC |
| | | (SEQ ID NO: 298) | (SEQ ID NO: 300) |
| | | ATAAGTAGCTGAAATATCTATTCT | CCCTCAACCCAGCTATGGTTCTCTC |
| | | GTATTATTGTGTTAACATTGAGAA | GCAAGGGAAGTCCTTGCAAGCTAAT |
| | | TAAGCCTTGGAA | TCTTTGACCT |
| | | (SEQ ID NO: 299) | (SEQ ID NO: 301) |
| NM_00103728 | CMTM5 | TGAGACGTCACTGGGGACTTATCT | GCAGTAGGGGGCTGTGTTGGTGGGC |
| 8 | | GTGGAGCCTGGTGCTCCAGGATGT | CCTACGAAGATGCTCAGTGCTCGA |
| | | GGCTTCTCATGA | GATCGCCGGGA |
| | | (SEQ ID NO: 302) | (SEQ ID NO: 304) |
| | | GGCATCCTGCTGGAAACCGAGCTG | CCTAGGCCCAGCCAGCCAGAGAGGA |
| | | GCCCTGACCCTCATCATCTTCATC | CAGTGGAGCCCAGACACGTCTCCT |
| | | TGCTTCACGGCC | TGGGATTCACT |
| | | (SEQ ID NO: 303) | (SEQ ID NO: 305) |
| NM_005564 | LCN2 | GCTATGGTGTTCTTCAAGAAAGTT | CAAAAGATGTATGCCACCATCTATG |
| | | TCTCAAAACAGGGAGTACTTCAAG | AGCTGAAAGAAGACAAGAGCTACAA |
| | | ATCACCCTCTAC | TGTCACCTCC |
| | | (SEQ ID NO: 306) | (SEQ ID NO: 308) |
| | | CTGAAAACCACATCGTCTTCCCTG | TCTGCATGCCCAGGCCCAGGACTCC |
| | | TCCCAATCGACCAGTGTATCGACG | ACCTCAGACCTGATCCCAGCCCCAC |
| | | GCTGAGTGCACA | CTCTGAGCAA ( |
| | | (SEQ ID NO: 307) | SEQ ID NO: 309) |
| XM_0115330 | NLRC4 | TTCTTAAATCCCTTAAGGAGTGGA | CCTCTGTGATCAACTCCTGGATATA |
| 08.1 | | ACTATCCTCTATTTCAGGACTTGA | CCTGGCACAATCAGGAAGCAGACAT |
| | | ATGGACAAAGTC | TCATGGCCAT |
| | | (SEQ ID NO: 310) | (SEQ ID NO: 312) |
| | | AACTTGAAAAGCACCTTCACAGAA | GGAATCTCATGAAGACCCCTCTCTT |
| | | CCTGTCCTGTGGAGGAAGGACCAA | TGTGGTCATCACTTGTGCAATCCAG |
| | | CACCATCACCGC | ATGGGTGAAA |
| | | (SEQ ID NO: 311) | (SEQ ID NO: 313) |
| NM_002704 | PPBP | GACAGTGACTTGTATGCTGAACTC | GTCGAAGTGATAGCCACACTGAAGG |
| | | CGCTGCATGTGTATAAAGACAACC | ATGGGAGGAAAATCTGCCTGGACCC |
| | | TCTGGAATTCAT | AGATGCTCCC |
| | | (SEQ ID NO: 314) | (SEQ ID NO: 316) |
| | | AGATGAAAATAAATAAGCCTGGTT | CTCAGTGTCTTAGTCCTAGGATGTC |
| | | TCAACCCTCTAATTCTTGCCTAAA | TTATTTAAAATACTCCCTGAAAGTT |
| | | CATTGGACTGTA | TATTCTGATG |
| | | (SEQ ID NO: 315) | (SEQ ID NO: 317) |
| NM_178174 | TREML1 | TCTGCTCCAAGCCTGTGACATATG | TCAGACTCCATCCAGCTAAGCTGCT |
| | | CCACAGTAATCTTCCCGGGAGGGA | CATCACACTTTAAACTCATGAGGAC |
| | | ACAAGGGTGGAG | CATCCCTAGG |
| | | (SEQ ID NO: 318) | (SEQ ID NO: 320) |
| | | TCTTAATGGCTGAATGGGAAAGGA | GAATTGCCTTTGGATGTACCACACA |
| | | AACTGCCCAAGTTTGACTAATTGC | TTAGGCTTGACTCACCACCTTCATT |
| | | TTGGCCTGTGAA | TGACAATACC |
| | | (SEQ ID NO: 319) | (SEQ ID NO: 321) |
| NM_002619 | PF4 | GACCTGCAAGCCCCGCTGTACAAG | GATTGCAGTACTTTATAGCTACATA |
| | | AAAATAATTAAGAAACTTTTGGAG | TTTACCTTGACCATTATTATTACCT |
| | | AGTTAGCTACTA | TTGCCAATAA |
| | | (SEQ ID NO: 322) | (SEQ ID NO: 324) |
| | | TTTGAAGGAAGGTGTGAATACTGG | CAATCTAACTGTGAAAGAACTTCTG |
| | | TTATGCTTGGTGTTACATGTTGGC | ATATTTGTGTTATCCTTATGATTTT |
| | | TGATACATATTC | AAATAAACAA |
| | | (SEQ ID NO: 323) | (SEQ ID NO: 325) |
| NM_016509 | CLEC1B | AAACATTATTTAATGTGTGAGAGG | GGTCTGTCATGCAGCGCAATTACCT |
| | | AAGGCTGGCATGACCAAGGTGGAC | ACAAGGTGAGAATGAAAATCGCACA |
| | | CAACTACCTTAA | GGAACTCTGC |
| | | (SEQ ID NO: 326) | (SEQ ID NO: 328) |
| | | GGTGGACAGGATAACACAGATAAG | TGTGACACAAACTGGAGATATTATG |
| | | GGCTTTATTGTACAATAAAAGATA | GAGATAGCTGCTATGGGTTCTTCAG |
| | | TGTATGAATGCA | GCACAACTTA |
| | | (SEQ ID NO: 327) | (SEQ ID NO: 329) |
| NM_203347 | LCN15 | GCCTGTGGGATGCCTTGTGGGACG | CAGCACCATGGTGCAGCTCTACAGC |
| | | TCTCTTTCTATTCAATAAACAGAT | CGGACCCAGGATGTGAGTCCCCAG |
| | | GCTGCAGCCTCA | GCTCTGAAGTC |
| | | (SEQ ID NO: 330) | (SEQ ID NO: 332) |
| | | TGCGCATCGTGGACACAGACTACA | CGGCTGTAACCAGGTGGATGCCGAG |
| | | GCTCCTTCGCCGTCCTTTACATCT | TACCTGAAGGTGGGCTCCGAGGGA |
| | | ACAAGGAGCTGG | CACTTCAGAGT |
| | | (SEQ ID NO: 331) | (SEQ ID NO: 333) |
| NM_172369 | C1QC | GCAGCGGCGTCAAAGTGGTCACCT | AGTCACTGCTTGTGTGGTTCCTGGG |
| | | TCTGTGGCCACACGTCCAAAACCA | ACACTTAACCAATGCCTTCTGGTAC |
| | | ATCAGGTCAACT | TGCCATTCTT |
| | | (SEQ ID NO: 334) | (SEQ ID NO: 336) |
| | | CATGGTGGGCATCCAGGGCTCTGA | CCTTCCACCTCCCTCAGCTTCCTGC |
| | | CAGCGTCTTCTCCGGCTTCCTGCT | ATGGACCCACCTTACTGGCCAGTCT |
| | | CTTCCCCGACTA | GCATCCTTGC |
| | | (SEQ ID NO: 335) | (SEQ ID NO: 337) |
| NM_000491 | C1QB | CACCGACAAGAACTCACTACTGGG | GACCAGACCATCCGCTTCGACCACG |
| | | CATGGAGGGTGCCAACAGCATCTT | TGATCACCAACATGAACAACAATTA |
| | | TTCCGGGTTCCT | TGAGCCCCGC |
| | | (SEQ ID NO: 338) | (SEQ ID NO: 340) |
| | | GCCAGCAACGCTCACTCTACCCCC | CCTCATGCGTGGCCGGGAGCGTGCA |
| | | AACACCACCCCTTGCCCAACCAAT | CAGAAGGTGGTCACCTTCTGTGACT |
| | | GCACACAGTAGG | ATGCCTACAA |
| | | (SEQ ID NO: 339) | (SEQ ID NO: 341) |
| NM_013363 | PCOLCE2 | TATCTGATTGGAAACCTGCCGACT | CATCAACATCTACAAAGAGGGAAAT |
| | | TAGTGCGGTGATAGGAAGCTAAAA | TTGGCGATTCAGCAGGCGGGCAAGA |
| | | GTGTCAAGCGTT | ACATGAGTGC |
| | | (SEQ ID NO: 342) | (SEQ ID NO: 344) |
| | | AACTGTGTCCATTTAAGCTGTATT | AGTGTAGACGGACGGGGACTCTGGA |
| | | CTGCCATTGCCTTTGAAAGATCTA | GGGCAATTATTGTTCAAGTGACTTT |
| | | TGTTCTCTCAGT | GTATTAGCCG |
| | | (SEQ ID NO: 343) | (SEQ ID NO: 345) |
| NM_015991 | C1QA | GGGTGACCAGGTCTGGGTTGAAAA | TCCCCCACCCACCTCTCTGGCTTCC |
| | | AGACCCCAAAAAGGGTCACATTTA | ATGCTCCGCCTGTAAAATGGGGGCG |
| | | CCAGGGCTCTGA | CTATTGCTTC |
| | | (SEQ ID NO: 346) | (SEQ ID NO: 348) |
| | | CTGCACTGTACCCGGCTACTACTA | GCAGCCCAGGAAACATCAAGGACCA |
| | | CTTCACCTTCCAGGTGCTGTCCCA | GCCGAGGCCAGCCTTCTCCGCCATT |
| | | GTGGGAAATCTG | CGGCGGAACC |
| | | (SEQ ID NO: 347) | (SEQ ID NO: 349) |
| NM_183240 | TMEM37 | AGCACGTCTGTACTTCTGTTCATT | ATCCCTGGGGCTCCCAGGGTTGTTA |
| | | AAAGTGCTCCCTTTCTAGTCCTTT | AGAATGGATCATTCTTCCAGCTAAG |
| | | TTCTGCCCAGAA | GGTCCAATCA |
| | | (SEQ ID NO: 350) | (SEQ ID NO: 352) |
| | | TCTAGACCTGCTGGACTCTGCAGG | TTCCTGAACGCCATCAGCGGCCTTC |
| | | GGGTGAGGGGGAACAGCGAGAGCT | ACATCAACAGCATCACCCATCCCTG |
| | | TGGGTAATGATT | GGAATGACCG |
| | | (SEQ ID NO: 351) | (SEQ ID NO: 353) |
| NM_000594 | TNF | GGGGTATCCTGGGGGACCCAATGT | CAGCCCTCCCCATGGAGCCAGCTCC |
| | | AGGAGCTGCCTTGGCTCAGACAT | CTCTATTTATGTTTGCACTTGTGAT |
| | | GTTTTCCGTGAAA | TATTTATTAT |
| | | (SEQ ID NO: 354) | (SEQ ID NO: 356) |
| | | CCAGAACTCACTGGGGCCTACAGC | AGGGGAGTTGTGTCTGTAATCGCCC |
| | | TTTGATCCCTGACATCTGGAATCT | TACTATTCAGTGGCGAGAAATAAAG |
| | | GGAGACCAGGGA | TTTGCTTAGA |
| | | (SEQ ID NO: 355) | (SEQ ID NO: 357) |
| NM_022154 | SLC39A8 | GGAAAATGATTGACAAAGCCCAAC | TCAAGAGCAAGTACATCAAAATGTA |
| | transcript | AATGATCTCAGGAATTACATTTTC | GAAGGTAAAATGTATGCAACACTAA |
| | variant 1 | CAACAGACCAAA | TATAAATTAT |
| | | (SEQ ID NO: 358) | (SEQ ID NO: 360) |
| | | ACCATGTGTTTGCTTTGTGAAGGT | AATTAGCACACCATGGTTATTTTTC |
| | | GAAGAATATGTTGGTTTAGAGAAA | TACCTTTTATAAAAGACAGAGCCTG |
| | | GAAATTGGATGT | TTTACTCATT |
| | | (SEQ ID NO: 359) | (SEQ ID NO: 361) |
| NM_0011351 | SLC39A8 | GGACATTAAAGGAATTTCCCTTTT | TCTGAACCTGGCCCTCTGAAACTTC |
| 47 | transcript | GTTGACATAACAGTAGGATGGCTA | CTGTTCCTGGACATCCCAGCTGCTA |
| | variant 3 | TAGCTAACAATA | ATGTGAGTTG |
| | | (SEQ ID NO: 362) | (SEQ ID NO: 364) |
| | | CTACTGTCCTTTGTGCAACCAGTA | GCCATTTTTATACTTTACTTATGGT |
| | | TAGAAGCGATTGACTCTCACTGCA | AGAGTTAAACTTCGTACTCTTAGGA |
| | | TCCTTGGGTCTT | GGCTACATGC |
| | | (SEQ ID NO: 363) | (SEQ ID NO: 365) |
| NM_012219 | MRAS | TTGTGTTTGAAATTCCATGTCGGG | AGTGGGCCCTCATGACTGAGGTAGC |
| | | TTTACTTGGAATGAAAGATACTTG | TTCCAGATAGGCCAGAGTAGAGTGT |
| | | AATTATTGTGCG | AGAGTGTGCC |
| | | (SEQ ID NO: 366) | (SEQ ID NO: 368) |
| | | TAGTGCTACCCTCACATCCCCTGG | GCCACTCGGGACACCTGGATGGTTT |
| | | AGCACAGCCTTCCTGAAATGCCCT | CTCTTAGGACTTTGCCCACCTCCTT |
| | | CACCCCATGCCT | CTCATGGCAC |
| | | (SEQ ID NO: 367) | (SEQ ID NO: 369) |
| NM_001548 | IFIT1 | ACTTTGAGAACTCTGTGAGACAAG | CATTATTCTTACCACAGAGCACCCC |
| | | GTCCTTAGGCACCCAGATATCAGC | AAGAAAATCTCCAAATTTTGGGCTT |
| | | CACTTTCACATT | CCAATCCATT |
| | | (SEQ ID NO: 370) | (SEQ ID NO: 372) |
| | | GAGCCAAAGTCAAGGTATTGATGA | CATATTCTTCCCAAACCTCATGCAG |
| | | CGCTACACTCCTCCGGAGGCTCTA | TTTACAATCTAGTGAGAGACACAGA |
| | | GGCAGATAGCCT | TAGCAGTACA |
| | | (SEQ ID NO: 371) | (SEQ ID NO: 373) |
| NM_006417 | IFI44 | AAGGATGTTCTAATTCTTTCTGCT | ATTAAAATTCAGAAAGGAGAAAACA |
| | | CTGAGACGAATGCTATGGGCTGCA | CAGACCAAAGAGAAGTATCTAAGAC |
| | | GATGACTTCTTA | CAAAGGGATG |
| | | (SEQ ID NO: 374) | (SEQ ID NO: 376) |
| | | AAGGTGACCTTATAGAAATAGAGA | AATTCGAAGGGAGTTGGTAAACGCT |
| | | GATGTGAGCCTGTGAGGTCCAAGC | GGTGTGGTACATGTGGCTTTGCTCA |
| | | TAGAGGAAGTCC | CTCATGTGGA |
| | | (SEQ ID NO: 375) | (SEQ ID NO: 377) |
| NM_020366 | RPGRIP1 | CCGAAGGCGGTTTCTGTTCGACAT | GCTCGATTCCCAGTGCTTGTGACCT |
| | | GCTGAATGGACAAGATCCTGATCA | CTGACCTGGACCATTATCTGAGACG |
| | | AGGACATTTAAA | GGAGGCCTTG |
| | | (SEQ ID NO: 378) | (SEQ ID NO: 380) |
| | | GGCAGATCCTGGAGTCAGGAAGAG | TCCCTACATACCCCCTGAGAGCTTC |
| | | ATATTCTAGAGCAAGAGCTAGACA | CTGAAACCAGAAGCTCAGACTAAGG |
| | | TTGTTAGCCCTG | GGAAGGATAC |
| | | (SEQ ID NO: 379) | (SEQ ID NO: 381) |
| NM_0011645 | DISC1 | GTGAATTTGAGAAATGTCAGACTG | CCACCTGTCTGCCCATGGTGTGATG |
| 54 | | TGCCAAAGGGGGTCACATGCATCA | TATCTATAATCCACTTGTTCATAAA |
| | | AATTTCCACATA | AAATATTCGT |
| | | (SEQ ID NO: 382) | (SEQ ID NO: 384) |
| | | TGATATGCTAGGGAATATGGGAGA | GTCATCATGTCCCAATTTTCTTTCC |
| | | CAATAGGAAAGTAAGACAGACATG | ATCTTTACTCATATCTACCTTTTGA |
| | | GTATCTGCCCTT | ATCCCAAAAG |
| | | (SEQ ID NO: 383) | (SEQ ID NO: 385) |
| NM_000634 | CXCR1 | CCGAAGGCGGTTTCTGTTCGACAT | CCTGCCTCACACCTTTGGCTTCATC |
| | | GCTGAATGGACAAGATCCTGATCA | GTGCCGCTGTTTGTCATGCTGTTCT |
| | | AGGACAT | GCTATGGATT |
| | | (SEQ ID NO: 386) | (SEQ ID NO: 388) |
| | | GGGGACTGTCTATGAATCTGTCCC | GTCCATTGGGCAGGCAGATGTTCCT |
| | | TGCCCTTCTTCCTTTTCCGCCAGG | AATAAAGCTTCTGTTCCGTGCTTGT |
| | | CTTACCATCCAA | CCCTGTGGAA |
| | | (SEQ ID NO: 387) | (SEQ ID NO: 389) |
| NM_177551 | HCAR2 | ATTGTCAAGGTGTTGCCGCCGGTG | AGATGACAGGTGAGCCAGATAATAA |
| | | TTGGGGCTGGAGTTTATCTTCGGG | CCGCAGCACGAGCGTCGAGCTCACA |
| | | CTTCTGGGCAAT | GGGGACCCCA |
| | | (SEQ ID NO: 390) | (SEQ ID NO: 392) |
| | | GTGGGAGACTGGTTGCAAGGTGTG | AGCAGAAATGGACTCAGGGAAGAGA |
| | | ACCGCAGGAATCCTGGAGGAATAG | CTCACACGCTTTGGTTAATATCTGT |
| | | AGAGTAAAGCTT | GTTTCCGGTG |
| | | (SEQ ID NO: 391) | (SEQ ID NO: 393) |
| NM_033255 | EPSTI1 | AGAAGAGAAGCATTTAGAGAGCAT | GGCCACATTGGTGGGTGGGGGGTAA |
| | | CAGCAATACAAAACCGCTGAGTTC | CAGAGAACAGATGGTGTCAGGAAG |
| | | TTGAGCAAACTG | TTTCTCTGGAG |
| | | (SEQ ID NO: 394) | (SEQ ID NO: 396) |
| | | CTACCTTACAGTTGTGTTCCGATT | TAACCAGGAGAGAAGCAAGACTTGC |
| | | TTTGCGTCTATGCTTGGTGTGCCT | TGCCTAAAGGAGCCCACCATTTTAC |
| | | CACTTGCTGCAT | TTTTCACATT |
| | | (SEQ ID NO: 395) | (SEQ ID NO: 397) |
| NM_0001278 | LILRB4 | TGAAGCCTTCTTCCTCCCAGGAAA | ATGGAGACTCAGGACCCCAGAAGGC |
| 26 | | GGGGACGTTCAGCTGAGCCGAGTG | ATGGAAGCTGCCTCCAGTAGACATC |
| | | TGTATACTGCTC | ACTGAACCCC |
| | | (SEQ ID NO: 398) | (SEQ ID NO: 400) |
| | | AAATATTACACATCAAACCAATGA | AAGAACACACAGCCTGAGGACGGGG |
| | | CATGGGAAAATGGGAGCTTCTAAT | TGGAAATGGACACTCGGCAGAGCC |
| | | GAGGACAAACAA | CACACGATGAA |
| | | (SEQ ID NO: 399) | (SEQ ID NO: 401) |
| NM_006840 | LILRB5 | CTAGATTCTGCAGTCAAAGATGAC | TCAGGAGGAAATGTGACCCTCCAGT |
| | | TAATATCCTTGCATTTTTGAAATG | GTGATACACTGGACGGACTTCTCAC |
| | | AAGCCACAGACT | GTTTGTTCTT |
| | | (SEQ ID NO: 402) | (SEQ ID NO: 404) |
| | | CCAAGGCCAAGTTCCACATTCCAT | TGTCTAAAGTCAAAGTACCAGTCTT |
| | | CCACGGTGTATGACAGTGCAGGGC | ATAGACACCAGGCTGAATTCTCCAT |
| | | GATACCGCTGCT | GAGTCCTGTG |
| | | (SEQ ID NO: 403) | (SEQ ID NO: 405) |
| NM_022351 | NECAB1 | ACGGCTCATGTTTTTAAAATATAT | ACTTCCCAATTTATGCAGGAAACCT |
| | | GTAACTCATTTTAAAATATATTAA | CCTGCAAAGCTGAAACTGATTAGAA |
| | | ATTGTATTCCAA | AATTCTTTAT |
| | | (SEQ ID NO: 406) | (SEQ ID NO: 408) |
| | | TCCATTACAGTTATTGTTGCTAGA | GTAGCAAATATTTTATATTTGAGAG |
| | | TCCACCTCATTTGCAGATGTCCAA | TCTTTACAGCTTACATTTCCATTCC |
| | | ACTTAAATTCAT | ATTATTACAA |
| | | (SEQ ID NO: 407) | (SEQ ID NO: 409) |
| NM_019065 | NECAB2 | TTTTTTCTAGACAGACACTTTGGT | TGCAGCTGGTCCGGCAGGAGATGGC |
| | | GCAGAAGCTTCTTTTCAATCCATC | CGTGTGCCCCGAGCAACTGAGCGA |
| | | CTCCACAAGAAG | GTTTCTGGACT |
| | | (SEQ ID NO: 410) | (SEQ ID NO: 412) |
| | | AGCACCCCTGCCTCCTGGTCCTGG | CTGGGAGACAGAGGAGGCGTGGAAG |
| | | CCTCTCCCCTACCCCTCACATGGC | AGGCACCTGCAGAGCCCCCTGTGT |
| | | CACGCATGACCC | AAGGCGTTCCG |
| | | (SEQ ID NO: 411) | (SEQ ID NO: 413) |
| NM_138694 | PKHD1 | GTACGTGTCAATAACAGTAAAGAA | TTCCATCTATCAGTCTTACTCCAAA |
| | | TTTTAATTAATTCTGTCATTTAAA | TATATGAAATTACTATACATTGAAA |
| | | ATTCATGCATTC | TGCCTGCAAA |
| | | (SEQ ID NO: 414) | (SEQ ID NO: 416) |
| | | ACCAGTTTCTGTATTTCCTAAAAC | AATTAGGGAAACACCAAAAGCAAAG |
| | | AGAGGCAGAATGGACTGCATCCTT | AGAGCAGCTCTATGTTCATTCATGT |
| | | CTTCAACGCAGG | CTGGAGATGA |
| | | (SEQ ID NO: 415) | (SEQ ID NO: 417) |
| NM_000296 | PKD1 | CTATGCGCTATGGAGAGAGTTCCT | AAGACACTGGTGCTGGATGAGACCA |
| | | CTTCTCCGTTCCCGCGGGGCCCCC | CCACATCCACGGGCAGCGCAGGCA |
| | | CGCGCAGTACTC | TGTGACTGGTG |
| | | (SEQ ID NO: 418) | (SEQ ID NO: 420) |
| | | ACGTGAGCAACGTCACCGTGAACT | TGGCTGTGCCTGCTTCTGTGTACCA |
| | | ACAACATCACCGTGGAGCGGATGA | CTTCTGTGGGCATGGCCGCTTCTAG |
| | | ACAGGATGCAGG | AGCCTCGACA |
| | | (SEQ ID NO: 419) | (SEQ ID NO: 421) |

Table 14 lists various probes used to detect the various biomarkers of the invention. Preferred probe sequences are shown in bold.

TABLE 15

| Biomarker | Forward primer | Reverse primer | Probe |
|---|---|---|---|
| FAM20A | GCCATCTTCGACTTCTTGATAGG | CCCGAACTTGGTGAACATCTC | AATATGGACCGGCACCAT |
| | (SEQ ID NO: 422) | (SEQ ID NO: 423) | (SEQ ID NO: 424) |
| | AGCGGCTCCTCAATGTCATC | CCGGTCCATATTCCCTATCAAG | CATGGCCATCTTCGACT |
| | (SEQ ID NO: 425) | (SEQ ID NO: 426) | (SEQ ID NO: 427) |
| OLAH | ACATGGAAGCCTGGAAAGATGT | CCCCTGGAAGCTGGTAAATTT | ACCAGTGGAAATGC |
| | (SEQ ID NO: 428) | (SEQ ID NO: 429) | (SEQ ID NO: 430) |
| | TTTGTCAAGTGCAACTCCTGTACA | GACAATTCATCATCTTTGGGAATG | TCAAAGGCCTGGCATC |
| | (SEQ ID NO: 431) | (SEQ ID NO: 432) | (SEQ ID NO: 433) |
| LCN2 | TGGGCCTCCCTGAAAACC | CCGTCGATACACTGGTCGATT | CATCGTCTTCCCTGTCC |
| | (SEQ ID NO: 434) | (SEQ ID NO: 435) | (SEQ ID NO: 436) |
| | GTTTCTCAAAACAGGGAGTACTTCAA | CTTTAGTTCCGAAGTCAGCTCCTT | ATCACCCTCTACGGGAGA |
| | (SEQ ID NO: 437) | (SEQ ID NO: 438) | (SEQ ID NO: 439) |
| ITGB3 | GTCCTCCAGCTCATTGTTGATG | GGTCACGCACTTCCAGCTCTA | TTATGGGAAAATCCGTTCTAA |
| | (SEQ ID NO: 440) | (SEQ ID NO: 441) | (SEQ ID NO: 442) |
| | ACGAAAATACCTGCAACCGTTAC | TTGCCAGTGTCCTTAAGCTCTTT | CGTGACGAGATTGAGTC |
| | (SEQ ID NO: 443) | (SEQ ID NO: 444) | (SEQ ID NO: 445) |
| MYL9 | GAGCCCAAGCGCCTTCTC | CCCGCTTGCTGGACATCTT | ACCAGGGAAGCCCCA |
| | (SEQ ID NO: 446) | (SEQ ID NO: 447) | (SEQ ID NO: 448) |
| | GCCGGCCCAGTTCCA | CTTCCCTGGTGCGGAGAA | ACCCAGCGAGCCCA |
| | (SEQ ID NO: 449) | (SEQ ID NO: 450) | (SEQ ID NO: 451) |
| TREML1 | ACCCAGCCAGGATGAGAAGA | GCCACCAGCAGACCTACCA | ATCCCCTTGATCTGGG |
| | (SEQ ID NO: 452) | (SEQ ID NO: 453) | (SEQ ID NO: 454) |
| | CAGTGCCAACCCTTTGGAA | GGAGCACAGCACCCCAGAT | CCAGCCAGGATGAG |
| | (SEQ ID NO: 455) | (SEQ ID NO: 456) | (SEQ ID NO: 457) |
| ITGA2B | CTGGGCAACCCCATGAAG | CCCCACGCTCACCAACAT | AACGCCCAGATAGGA |
| | (SEQ ID NO: 458) | (SEQ ID NO: 459) | (SEQ ID NO: 460) |
| | CCCTTCGAGGTGCCGTAGA | CCGTAAGCTCCCACGATCAG | TCGATGACAACGGATACC |
| | (SEQ ID NO: 461) | (SEQ ID NO: 462) | (SEQ ID NO: 463) |
| PLA2G7 | TGTTGCCCATATGAAATCATCAG | AAGCTTGCAGCAGCCATCA | ATGGGTCAACAAAATACAAGT |
| | (SEQ ID NO: 464) | (SEQ ID NO: 465) | (SEQ ID NO 466) |
| | ACACTGGCTTATGGGCAACAT | ATTCCAGTTTGCAGGAGTTGTCA | TTGAGGTTACTCTTTGGTTCA |
| | (SEQ ID NO: 467) | (SEQ ID NO: 468) | (SEQ ID NO: 469) |
| ARHGEF10L | ATCCACTCGGCCAACAAGTG | GCCGGACTTGTCGGGTTT | CGTCTCAGGCTCCTG |
| | (SEQ ID NO: 470) | (SEQ ID NO: 471) | (SEQ ID NO: 472) |
| TGFBI | GGTCCATGTCATCACCAATGTT | CCCCTCTTTCCTGAGGTCTGT | TGCAGCCTCCAGCC |
| | (SEQ ID NO: 473) | (SEQ ID NO: 474) | (SEQ ID NO: 475) |
| | GGCGTGGTCCATGTCATCA | CCCTCTTTCCTGAGGTCTGTTG | TGTTCTGCAGCCTCC |
| | (SEQ ID NO: 476) | (SEQ ID NO: 477) | (SEQ ID NO: 478) |
| MYCL | GCTGGGCGAACCCAAGA | TCTTCTCTACTGTCACAACATCAATTTC | CCAGGCCTGCTCC |
| | (SEQ ID NO: 479) | (SEQ ID NO: 480) | (SEQ ID NO: 481) |
| | GCTGGGCGAACCCAAGA | TCTTCTCTACTGTCACAACATCAATTTC | CGACTCGGAGAATGA |
| | (SEQ ID NO: 482) | (SEQ ID NO: 483) | (SEQ ID NO: 484) |
| CXCR1 | GACTGCAGCTCCTACTGTTGGA | TTCAGCAATGGTTTGATCTAACTGA | ACACCTGGCCGGTGC |
| | (SEQ ID NO: 485) | (SEQ ID NO: 486) | (SEQ ID NO: 487) |
| | CCTGGCCGGTGCTTCAG | TCTGTAATATTTGACATGTCCTCTTCAG | AGATCAAACCATTGCTG |
| | (SEQ ID NO: 488) | (SEQ ID NO: 489) | (SEQ ID NO: 490) |
| HCAR2 | TTAGGGAAACGGTGGCAGAT | GATTCCTGCGGTCACACCTT | AGTGGGAGACTGGTTG |
| | (SEQ ID NO: 491) | (SEQ ID NO: 492) | (SEQ ID NO: 493) |
| | TTCACCTACATGAACAGCATGCT | GGAAAGGATGGGCTGGAGAA | ACCCCGTGGTGTACTA |
| | (SEQ ID NO: 494) | (SEQ ID NO: 495) | (SEQ ID NO:496) |
| C1QA | GCGGCCAGGCCTCAA | CCCCCTGGTCTCCTTTAAGG | TCCGGACAGGCATC |
| | (SEQ ID NO: 497) | (SEQ ID NO: 498) | (SEQ ID NO: 499) |
| C1QB | GCCTCACAGGACACCAGCTT | CCCATGGGATCTTCATCATCA | CCAGGAGGCGTCTGA |
| | (SEQ ID NO: 500) | (SEQ ID NO: 501) | (SEQ ID NO: 502) |
| | | TGCCCCATGGGATCTTCAT | |
| | | (SEQ ID NO: 503) | |
| C1QC | TGTGCCAGGCCAGAAACC | AGCAGCTTCAGCCCAAGGT | CCTTCTCCGGGATGGA |
| | (SEQ ID NO: 504) | (SEQ ID NO: 505) | (SEQ ID NO: 506) |
| | GAAGCAGATCTGAGGACATCTCTGT | CGGGAATGGCTGGGATTC | CCGCCCACCTGCA |
| | (SEQ ID NO: 507) | (SEQ ID NO: 508) | (SEQ ID NO: 509) |
| MRAS | CACCAGGGAGCAAGGAAAAG | GGCACTGGTTTCTATGTACGGAAT | TGGCGACCAAACACAA |
| | (SEQ ID NO: 510) | (SEQ ID NO: 511) | (SEQ ID NO: 512) |
| | CAATGTCGACAAAGCCTTCCA | GGCTTTTTTCCGGAATCTGTT | ACCTCGTTAGAGTAATTAG |
| | (SEQ ID NO: 513) | (SEQ ID NO: 514) | (SEQ ID NO: 515) |
| SLC39A8 | GCACTTGCTGGAGGCATGT | TCAGCATATCATTCATCTCTGGAAA | CCTCTATATTTCTCTGGCAGATA |
| | (SEQ ID NO: 516) | (SEQ ID NO: 517) | (SEQ ID NO: 518) |
| | GGGACTCAGTACTTCCATAGCAATC | TGCATTGAGTAGGATCACAAAGTCT | TGAGGAGTTTCCCCACGAG |
| | (SEQ ID NO: 519) | (SEQ ID NO: 520) | (SEQ ID NO: 521) |
| TMEM37 | CGGGCGCAGCATGACT | CCGGATGAAGGATTCAAAGAAG | CCGTCGGCGTGCAG |
| | (SEQ ID NO: 522) | (SEQ ID NO: 523) | (SEQ ID NO: 524) |
| | | | CCCAGAGGCCTTTG |
| | | | (SEQ ID NO: 525) |
| FCER1A | GGCAGCTGGACTATGAGTCTGA | CTTCTCACGCGGAGCTTTTATT | CCCCTCAACATTACTG |
| | (SEQ ID NO: 526) | (SEQ ID NO: 527) | (SEQ ID NO: 528) |
| | ACCGAGCATGGGCCTATATTT | CATGGACTCCTGGTGCTTACTG | AAGCCTTAGATCTCTCC |
| | (SEQ ID NO: 529) | (SEQ ID NO: 530) | (SEQ ID NO: 531) |
| BCL11B | GCAACCCGCAGCACTTGT | TGGCGGCCTCCACATG | CCAGAGGGAGCTCAT |
| | (SEQ ID NO: 532) | (SEQ ID NO: 533) | (SEQ ID NO: 534) |
| | TCCCAGAGGGAGCTCATCAC | TCTCCAGACCCTCGTCTTCTTC | AGGCTGACCATGTGGAG |
| | (SEQ ID NO: 535) | (SEQ ID NO: 536) | (SEQ ID NO: 537) |
| PKHD1 | TGAGGATCTATGAACGGCTCAA | CCATCCTCCGTGACATGTACAC | CACCGGCATATTGG |
| | (SEQ ID NO: 538) | (SEQ ID NO: 539) | (SEQ ID NO: 540) |
| | CATTACAACCCAGGAAAGATTTAGG | GAGATATTTTCTTGGACTTGCACAGT | AAAGTAGTCTGTCCTGAATTA |
| | (SEQ ID NO: 541) | (SEQ ID NO: 542) | (SEQ ID NO: 543) |
| KLRB1 | TTGGTTGTTACTGGGTTGAGTGTT | CCACACTGCATTTTTCTATTGATGA | CAGTGACATCCTTAATACAG |
| | (SEQ ID NO: 544) | (SEQ ID NO: 545) | (SEQ ID NO: 546) |
| | CAACAGAGCAGGAATAAAACAACAG | TCTCGGAGTTGCTGCCAATA | CCGGGTCTCTTAAAC |
| | (SEQ ID NO: 547) | (SEQ ID NO: 548) | (SEQ ID NO: 549) |
| LILRB5 | CCAGCCCAGTTGCTGACAT | TGTCCTTCACGGCAGCATT | CAGGAGGAAATTCT |
| | (SEQ ID NO: 550) | (SEQ ID NO: 551) | (SEQ ID NO: 552) |
| | CCAGGGCCTGCAGAAGAG | CGGCAGCATTGAGAATTTCC | CCAGCCCAGTTGCTGA |
| | (SEQ ID NO: 553) | (SEQ ID NO: 554) | (SEQ ID NO: 555) |
| NECAB1 | TCTACAATGCTAGTTCCTGCTTCGT | GCACTTGGAACCATAAAGAAAATGT | TCCTGAACAACTAGATGTT |
| | (SEQ ID NO: 556) | (SEQ ID NO: 557) | (SEQ ID NO: 558) |
| | ACAACAGCTTCTCCCCAAACA | CTGGGTCATCCACTGGTTGTC | ATGTCAGCGGTCCAGG |
| | (SEQ ID NO: 559) | (SEQ ID NO: 560) | (SEQ ID NO: 561) |
| NECAB2 | GTGGAGGCCATCGAGGAA | GCTGTGGCTGGGTTTGATGT | CAGCTCCGACAGAAC |
| | (SEQ ID NO: 562) | (SEQ ID NO: 563) | (SEQ ID NO: 564) |
| | GCCGTGCGGACAAAAATG | TCTGCAAAGAAGAGCTGGAATTC | TGATGGGAAGCTGTCC |
| | (SEQ ID NO: 565) | (SEQ ID NO: 566) | (SEQ ID NO: 567) |
| ALAS1 | GATGATGCCAGGCTGTGAGA | CGAATCCCTTGGATCATGGA | CTGATTCTGGGAACCAT |
| (control | (SEQ ID NO: 568) | (SEQ ID NO: 569) | (SEQ ID NO: 570) |
| gene) | AACCCTCTTCACCCTGGCTAA | GGCATGGTTCCCAGAATCAG | ATGATGCCAGGCTGTG |
| | (SEQ ID NO: 571) | (SEQ ID NO: 572) | (SEQ ID NO: 573) |
| HMBS | CCTGCCCACTGTGCTTCCT | GGTTTTCCCGCTTGCAGAT | CTGGCTTCACCATCG |
| (control | (SEQ ID NO: 574) | (SEQ ID NO: 575) | (SEQ ID NO: 576) |
| gene) | GCCTGTTTACCAAGGAGCTTGA | TGAACAACCAGGTCCACTTCAT | CATGCCCTGGAGAAG |
| | (SEQ ID NO: 577) | (SEQ ID NO: 578) | (SEQ ID NO: 579) |
| GTF2D1 | GCACTTCGTGCCCGAAAC | CCTCATGATTACCGCAGCAA | CGAATATAATCCCAAGCGG |
| (TBP) | (SEQ ID NO: 580) | (SEQ ID NO: 581) | (SEQ ID NO: 582) |
| (control | GCCCGAAACGCCGAATAT | CGTGGCTCTCTTATCCTCATGA | AGCGGTTTGCTGCGGT |
| gene) | (SEQ ID NO: 583) | (SEQ ID NO: 584) | (SEQ ID NO: 585) |

Table 15 lists various oligonucleotide primers and corresponding probes used to detect the biomarkers of the invention

### SEQUENCE INFORMATION

Set out below are the nucleotide sequences of the biomarkers described herein. Exemplary target regions within the biomarker sequences are underlined, and exemplary probe sequences are underlined and shown in bold text.

ADM (SEQ ID NO: 1) - Homo sapiens adrenomedullin (ADM),
mRNA - NM_001124

| | |
|---|---|
| 1 | gaggaaagaa agggaaggca accgggcagc ccaggccccg ccccgccgct cccccacccg |
| 61 | tgcgcttata aagcacagga accagagctg gccactcagt ggtttcttgg tgacactgga |
| 121 | tagaacagct caagccttgc cacttcgggc ttctcactgc agctgggctt ggacttcgga |
| 181 | gttttgccat tgccagtggg acgtctgaga ctttctcctt caagtacttg gcagatcact |
| 241 | ctcttagcag ggtctgcgct tcgcagccgg gatgaagctg gtttccgtcg ccctgatgta |
| 301 | cctgggttcg ctcgccttcc taggcgctga caccgctcgg ttggatgtcg cgtcggagtt |
| 361 | tcgaaagaag tggaataagt gggctctgag tcgtgggaag agggaactgc ggatgtccag |
| 421 | cagctacccc accgggctcg ctgacgtgaa ggccgggcct gcccagaccc ttattcggcc |
| 481 | ccaggacatg aagggtgcct ctcgaagccc cgaagacagc agtccggatg ccgcccgcat |
| 541 | ccgagtcaag cgctaccgcc agagcatgaa caacttccag ggcctccgga gctttggctg |
| 601 | ccgcttcggg acgtgcacgg tgcagaagct ggcacaccag atctaccagt tcacagataa |
| 661 | ggacaaggac aacgtcgccc ccaggagcaa gatcagcccc cagggctacg gccgccggcg |
| 721 | ccggcgctcc ctgcccgagg ccggcccggg tcggactctg gtgtcttcta agccacaagc |
| 781 | acacggggct ccagcccccc cgagtggaag tgctccccac tttctttagg atttaggcgc |
| 841 | ccatggtaca aggaatagtc gcgcaagcat cccgctggtg cctcccggga cgaaggactt |
| 901 | cccgagcggt gtggggaccg ggctctgaca gccctgcgga gaccctgagt ccgggaggca |
| 961 | ccgtccggcg gcgagctctg gctttgcaag ggcccctcct tctgggggct tcgcttcctt |
| 1021 | agccttgctc aggtgcaagt gccccagggg gcggggtgca gaagaatccg agtgtttgcc |
| 1081 | aggcttaagg agaggagaaa ctgagaaatg aatgctgaga cccccggagc aggggtctga |
| 1141 | gccacagccg tgctcgccca caaactgatt tctcacggcg tgtcacccca ccagggcgca |
| 1201 | agcctcacta ttacttgaac tttccaaaac ctaaagagga aaagtgcaat gcgtgttgta |
| 1261 | catacagagg taactatcaa tatttaagtt tgttgctgtc aagatttttt ttgtaacttc |
| 1321 | aaatatagag atatttttgt acgttatata ttgtattaag ggcattttaa aagcaattat |
| 1381 | attgtcctcc cctattttaa gacgtgaatg tctcagcgag gtgtaaagtt gttcgccgcg |
| 1441 | tggaatgtga gtgtgtttgt gtgcatgaaa gagaaagact gattacctcc tgtgtggaag |
| 1501 | aaggaaacac cgagtctctg tataatctat ttacataaaa tgggtgatat gcgaacagca |
| 1561 | aaccaataaa ctgtctcaat gctgattcat aaaaaaaaaa aaaaaa |

CD177 (SEQ ID NO: 2) - Homo sapiens CD117 molecule (CD177),
mRNA - NM_020406

| | |
|---|---|
| 1 | aaaggacttg tttcctgctg aaaaagcaga aagagattac cagccacaga cgggtcatga |
| 61 | gcgcggtatt actgctggcc ctcctggggt tcatcctccc actgccagga gtgcaggcgc |
| 121 | tgctctgcca gtttgggaca gttcagcatg tgtggaaggt gtccgacctg ccccggcaat |
| 181 | ggacccctaa gaacaccagc tgcgacagcg gcttggggtg ccaggacacg ttgatgctca |
| 241 | ttgagagcgg accccaagtg agcctggtgc tctccaaggg ctgcacggag gccaaggacc |
| 301 | aggagccccg cgtcactgag caccggatgg gccccggcct ctccctgatc tcctacacct |
| 361 | tcgtgtgccg ccaggaggac ttctgcaaca acctcgttaa ctccctcccg ctttgggccc |
| 421 | cacagccccc agcagaccca ggatccttga ggtgcccagt ctgcttgtct atggaaggct |
| 481 | gtctggaggg gacaacagaa gagatctgcc ccaaggggac cacacactgt tatgatggcc |
| 541 | tcctcaggct caggggagga ggcatcttct ccaatctgag agtccaggga tgcatgcccc |
| 601 | agccagtttg caacctgctc aatgggacac aggaaattgg gcccgtgggt atgactgaga |
| 661 | actgcgatat gaaagatttt ctgacctgtc atcgggggac caccattatg acacacggaa |
| 721 | acttggctca agaacccact gattggacca catcgaatac cgagatgtgc gaggtggggc |
| 781 | aggtgtgtca ggagacgctg ctgctcctag atgtaggact cacatcaacc ctggtgggga |
| 841 | caaaaggctg cagcactgtt ggggctcaaa attcccagaa gaccaccatc cactcagccc |
| 901 | ctcctggggt gcttgtggcc tcctataccc acttctgctc ctcggacctg tgcaatagtg |
| 961 | ccagcagcag cagcgttctg ctgaactccc tccctcctca agctgcccct gtcccaggag |
| 1021 | accggcagtg tcctacctgt gtgcagcccc ttggaacctg ttcaagtggc tccccccgaa |
| 1081 | tgacctgccc caggggcgcc actcattgtt atgatgggta cattcatctc tcaggaggtg |
| 1141 | ggctgtccac caaaatgagc attcagggct gcgtggccca accttccagc ttcttgttga |
| 1201 | accacaccag acaaatcggg atcttctctg cgcgtgagaa gcgtgatgtg cagcctcctg |
| 1261 | cctctcagca tgagggaggt ggggctgagg gcctggagtc tctcacttgg ggggtggggc |
| 1321 | tggcactggc cccagcgctg tggtggggag tggtttgccc ttcctgctaa ctctattacc |
| 1381 | cccacgattc ttcaccgctg ctgaccaccc acactcaacc tccctctgac ctcataacct |
| 1441 | aatggccttg gacaccagat tctttcccat tctgtccatg aatcatcttc cccacacaca |
| 1501 | atcattcata tctactcacc taacagcaac actggggaga gcctggagca tccggacttg |
| 1561 | ccctatggga gaggggacgc tggaggagtg gctgcatgta tctgataata cagaccctgt |
| 1621 | cctttctccc agtgctggga tttctccatg tgagggggca gcaggacacc cagggatcta |
| 1681 | gcgtggggga ggagaggagc ctaatgagaa aatgaccatc taaagcctgc ccttcattgg |
| 1741 | tctggttcac gtctccaaac cagcttggat ggtagcagag acttcagggt gctccagcca |
| 1801 | aacgtatttg ggcatcacca tgacctggga ggggaagatg cactgagacg tatgaggctt |
| 1861 | ccagcctagc agccagggcc ctagcacaaa caggaggctc gccccatctg agcaactgca |
| 1921 | ggagaggtta gtacagtcat gcattgctta acgacaggga cgtgtcgtta gaaatgtgtc |
| 1981 | gttaggtgat tttatgacca taggaacatt gtagcgtgca cttacaccaa cccagatggt |
| 2041 | acagcccaat acacacccag gatggacgct agagtcgact gctcctaggc tacaagcctg |
| 2101 | cagtgcatgt tatggtgtga atactgcagg caatcttaac accacggcaa gtatttgtgc |
| 2161 | atctacacac atctaaacat agaaaaggta cagcataaat acactattgt catctcagca |
| 2221 | gaaaaaaaaa aaaaaaaa |

FAM20A (SEQ ID NO: 3) - Homo sapiens family with sequence similarity
20 member A (FAM20A), transcript variant 1, mRNA - NM_017565

| | |
|---|---|
| 1 | aaaggcgcaa gaagcgggca ccccgggaac cccattccct cggctcactc ggcgcggaga |
| 61 | agcgacgccc gctgactccg agagccccgg tgctccgtgc acctggtccc caagttgagg |
| 121 | agcgacaccc ctccacaggg gactagcccg cgcggggagc attccggtct cactgacccc |
| 181 | ggcccacccg cgggactcca ggcacctctt ctgcccgcac cccgcgaccc ctcccgggac |
| 241 | cccggagaca gccggcctgc ccccggcgtc ccccttggcc agcacgccat gccggggctg |
| 301 | cgccgggacc gcctactgac tctgctgctg ctgggcgcgc tgctctccgc cgacctctac |
| 361 | ttccacctct ggccccaagt acagcgccag ctgcggcctc gggagcgccc gcgggggtgc |
| 421 | ccgtgcaccg gccgcgcctc ctccctggcg cgggactcgg ccgcagctgc ctcggacccc |
| 481 | ggcacgatcg tgcacaactt ttcccgaacc gagccccgga ctgaaccggc tggcggcagc |
| 541 | cacagcgggt cgagctccaa gttgcaggcc ctcttcgccc acccgctgta caacgtcccg |
| 601 | gaggagccgc ctctcctggg agccgaggac tcgctcctgg ccagccagga ggcgctgcgg |
| 661 | tattaccgga ggaaggtggc ccgctggaac aggcgacaca agatgtacag agagcagatg |
| 721 | aaccttacct ccctggaccc cccactgcag ctccgactcg aggccagctg ggtccagttc |
| 781 | cacctgggta ttaaccgcca tgggctctac tcccggtcca gccctgttgt cagcaaactt |
| 841 | ctgcaagaca tgaggcactt tcccaccatc agtgctgatt acagtcaaga tgagaaagcc |
| 901 | ttgctggggg catgtgactg cacccagatt gtgaaaccca gtggggtcca cctcaagctg |
| 961 | gtgctgaggt tctcggattt cgggaaggcc atgttcaaac ccatgagaca gcagcgagat |
| 1021 | gaggagacac cagtggactt cttctacttc attgactttc agagacacaa tgctgagatc |
| 1081 | gcagctttcc atctggacag gattctggac ttccgacggg tgccgccaac agtggggagg |
| 1141 | atagtaaatg tcaccaagga aatcctagag gtcaccaaga atgaaatcct gcagagtgtt |
| 1201 | ttctttgtct ctccagcgag caacgtgtgc ttcttcgcca agtgtccata catgtgcaag |
| 1261 | acggagtatg ctgtctgtgg caacccacac ctgctggagg gttccctctc tgccttcctg |
| 1321 | ccgtccctca acctggcccc caggctgtct gtgcccaacc cctggatccg ctcctacaca |
| 1381 | ctggcaggaa aagaggagtg ggaggtcaat cccctttact gtgacacagt gaaacagatc |
| 1441 | tacccgtaca acaacagcca gcggctcctc aatgtcatcg acatggccat cttcgacttc |
| 1501 | ttgataggga atatggaccg gcaccattat gagatgttca ccaagttcgg ggatgatggg |
| 1561 | ttccttattc accttgacaa cgccagaggg ttcggacgac actcccatga tgaaatctcc |
| 1621 | atcctctcgc ctctctccca gtgctgcatg ataaaaaaga aaacactttt gcacctgcag |
| 1681 | ctgctggccc aagctgacta cagactcagc gatgtgatgc gagaatcact gctggaagac |
| 1741 | cagctcagcc ctgtcctcac tgaaccccac ctccttgccc tggatcgaag gctccaaacc |
| 1801 | atcctaagga cagtggaggg gtgcatagtg gcccatggac agcagagtgt catagtcgac |
| 1861 | ggcccagtgg aacagttggc cccagactct ggccaggcta acttgacaag ctaagggctg |
| 1921 | gcagagtcca gtttcagaaa atacgcctgg agccagagca gtcgactcga gtgccgaccc |
| 1981 | tgcgtcctca ctcccacctg ttactgctgg gagtcaagtc agctaggaag gaagcaggac |
| 2041 | attttctcaa acagcaagtg gggcccatgg aactgaatct ttactccttg gtgcaccgct |
| 2101 | tctgtcgtgc gttgccttgc tccgtttttc ccaaaaagca ctggcttcat caaggccacc |
| 2161 | gacgatctcc tgagtgcact gggaaatctg ggtataggtc aggcttggca gccttgatcc |
| 2221 | caggagagta ctaatggtaa caagtcaaat aaaaggacat caagtggata cctgacttct |
| 2281 | caggatcctt attctagcta caagtcaaag ataactcctg gtccagacaa aacacctggc |
| 2341 | ctatcacaag ctgactaaaa atctgcactt tgggccagcg caggcaacag taactctgac |
| 2401 | aggttcaaat tagacctcac actttctact catattctag tcactggacc catctgaatc |
| 2461 | agtaatccct actgcccggt cctggagtaa cttcttagag atattataac aagtggcaaa |
| 2521 | aataaaagag ggatttgcta agaatatcag aaaaggagtg ttccaatttg aagagtatta |
| 2581 | caattgaaat aacatcaaat atgtcacact aagcagccag taacagaata aataattaca |
| 2641 | acgaaggaaa aaaaaaggaa agtcctccaa ggtcaggatg gcatgggaac aggcctagca |
| 2701 | gggacacaag cctggagtaa ggcaggaaaa gagccaaggc tgactacagc ccaccaacca |
| 2761 | caatcttctt ccctaagacc ccaggattgt ccccggccca tccccaaaag aagaaagggg |
| 2821 | ctatgtggaa aggtgaggcc ctctagatgc ccctccctgt gacggctggc tcaaaaaaga |
| 2881 | ctaagtcagg ttttttattg aagcctccct tagaagaaag tgtagtaggg attccttgtc |
| 2941 | tcctctacct tctccctgac ttgttgatat ctgaaactcc ttttaatagg aggctttgtt |
| 3001 | tcttattcca taattaatga ccgaaaacac aaaggacagt aaggtcttca ttccatggag |
| 3061 | tgactagtca gtcaacaaaa gtaaaaatgc ctcatttcat caaatcaact tttccagtaa |
| 3121 | aggccagagt tcaaatactg taagcatgag aatagaatgg agctgtcact aaagagagta |
| 3181 | caaggtcaag aaccagaaat gtctccaagc tttctgcagt gtgagcaaag atagcctcga |
| 3241 | ctggacaaat aacacttctg accccgtggt acccccacat gactggtatg ctgcccctgg |
| 3301 | actttgtgtc tctctaaaat cactaccatt cactgagatc tgaccaggtg aggcactcac |
| 3361 | caacgctaca gaagcaacac acaggtttcc aatcctctca aacacagata caacccccat |
| 3421 | cctccaatgt gcatggaaga actttaaatc tcagagagag taagcaaaat cacacaattt |
| 3481 | agtggtttag acctattcca aagtcttctt ttcactgtaa cacaaaaccc aacagtatcc |
| 3541 | cacccaattt atgaagtcaa atttgtttgc ttaaaaaaaa aatcagctgg gcatgggctc |
| 3601 | acacctgtaa tcccagcact ttgggcctaa acaggcagat cgcttggtct caggagctcg |
| 3661 | agaccagcct gggtaacatg acaaaaccct gtctctacaa aaaaatacaa aaaaaaaaaa |
| 3721 | attttttttt aaatcaactc tttaacagag aaaccttaat cccaaagatt tccttaactg |
| 3781 | ttgtggccct actgcagcat agggctcccc acttcattca ctgtctctcc tgcttcaaga |
| 3841 | tgtttcttcc ctgtggatga agaaccagga gatcacacac agaaatggga aaatatctgg |
| 3901 | gtaaaaacat tcaaagaata gcacaaaatg gtttgggact tccaaataag actctcagga |
| 3961 | aagcaggcta cagctctacc tcaaaacccg ccacctccta taacgaagga ctggttttcc |
| 4021 | aatggcctat atacacattt tgctgcactc tacaaacaga aagcaatagg cttgaatttc |
| 4081 | accatgatat gtggtacacc taccttatta ttcacttatc tttcaacctt ccattacttt |
| 4141 | ttgattagta acaaaattaa gactcatttc tcaaaaatat cagaaaacca accgtttcat |
| 4201 | tcttactcga gttcttccag catgttcatt ttgaactgtt tacctcccat gaaataaaaa |
| 4261 | gtctcttgac tctgaaaaaa aaaaa |

IL10 (SEQ ID NO: 4) - Homo sapiens interleukin 10 (IL10),
mRNA - NM_000572

| | |
|---|---|
| 1 | acacatcagg ggcttgctct tgcaaaacca aaccacaaga cagacttgca aaagaaggca |
| 61 | tgcacagctc agcactgctc tgttgcctgg tcctcctgac tggggtgagg gccagcccag |
| 121 | gccagggcac ccagtctgag aacagctgca cccacttccc aggcaacctg cctaacatgc |
| 181 | ttcgagatct ccgagatgcc ttcagcagag tgaagacttt ctttcaaatg aaggatcagc |
| 241 | tggacaactt gttgttaaag gagtccttgc tggaggactt taagggttac ctgggttgcc |
| 301 | aagccttgtc tgagatgatc cagttttacc tggaggaggt gatgccccaa gctgagaacc |
| 361 | aagacccaga catcaaggcg catgtgaact ccctggggga gaacctgaag accctcaggc |
| 421 | tgaggctacg gcgctgtcat cgatttcttc cctgtgaaaa caagagcaag gccgtggagc |
| 481 | aggtgaagaa tgcctttaat aagctccaag agaaaggcat ctacaaagcc atgagtgagt |
| 541 | ttgacatctt catcaactac atagaagcct acatgacaat gaagatacga aactgagaca |
| 601 | tcagggtggc gactctatag actctaggac ataaattaga ggtctccaaa atcggatctg |
| 661 | gggctctggg atagctgacc cagccccttg agaaacctta ttgtacctct cttatagaat |
| 721 | atttattacc tctgatacct caacccccat ttctatttat ttactgagct tctctgtgaa |
| 781 | cgatttagaa agaagcccaa tattataatt tttttcaata tttattattt tcacctgttt |
| 841 | ttaagctgtt tccatagggt gacacactat ggtatttgag tgttttaaga taaattataa |
| 901 | gttacataag ggaggaaaaa aaatgttctt tggggagcca acagaagctt ccattccaag |
| 961 | cctgaccacg ctttctagct gttgagctgt tttccctgac ctccctctaa tttatcttgt |
| 1021 | ctctgggctt ggggcttcct aactgctaca aatactctta ggaagagaaa ccagggagcc |
| 1081 | cctttgatga ttaattcacc ttccagtgtc tcggagggat tcccctaacc tcattcccca |
| 1141 | accacttcat tcttgaaagc tgtggccagc ttgttattta taacaaccta aatttggttc |
| 1201 | taggccgggc gcggtggctc acgcctgtaa tcccagcact ttgggaggct gaggcgggtg |
| 1261 | gatcacttga ggtcaggagt tcctaaccag cctggtcaac atggtgaaac cccgtctcta |
| 1321 | ctaaaaatac aaaaattagc cgggcatggt ggcgcgcacc tgtaatccca gctacttggg |
| 1381 | aggctgaggc aagagaattg cttgaaccca ggagatggaa gttgcagtga gctgatatca |
| 1441 | tgcccctgta ctccagcctg ggtgacagag caagactctg tctcaaaaaa taaaaataaa |
| 1501 | aataaatttg gttctaatag aactcagttt taactagaat ttattcaatt cctctgggaa |
| 1561 | tgttacattg tttgtctgtc ttcatagcag attttaattt tgaataaata aatgtatctt |
| 1621 | attcacatc |

METTL7B (SEQ ID NO: 5) - Homo sapiens methyltransferase like 7B
(METTL7B), mRNA - NM_152637

| | |
|---|---|
| 1 | aaaagtcatt gaagagcttg tggggctgtg ggacctgcgc cctctggagg aattccatac |
| 61 | acccactcaa ctctggcaaa taggaaattg tcaagtagga gacaaggagc aaagtcctat |
| 121 | cacagcggga ggggacgcca gcgcctgcag aggctgagca gggaaaaagc cagtgcccca |
| 181 | gcggaagcac agctcagagc tggtctgcca tggacatcct ggtcccactc ctgcagctgc |
| 241 | tggtgctgct tcttaccctg cccctgcacc tcatggctct gctgggctgc tggcagcccc |
| 301 | tgtgcaaaag ctacttcccc tacctgatgg ccgtgctgac tcccaagagc aaccgcaaga |
| 361 | tggagagcaa gaaacgggag ctcttcagcc agataaaggg gcttacagga gcctccggga |
| 421 | aagtggccct actggagctg ggctgcggaa ccggagccaa ctttcagttc tacccaccgg |
| 481 | gctgcagggt cacctgccta gacccaaatc cccactttga gaagttcctg acaaagagca |
| 541 | tggctgagaa caggcacctc caatatgagc ggtttgtggt ggctcctgga gaggacatga |
| 601 | gacagctggc tgatggctcc atggatgtgg tggtctgcac tctggtgctg tgctctgtgc |
| 661 | agagcccaag gaaggtcctg caggaggtcc ggagagtact gagaccggga ggtgtgctct |
| 721 | ttttctggga gcatgtggca gaaccatatg gaagctgggc cttcatgtgg cagcaagttt |
| 781 | tcgagcccac ctggaaacac attggggatg gctgctgcct caccagagag acctggaagg |
| 841 | atcttgagaa cgcccagttc tccgaaatcc aaatggaacg acagccccct cccttgaagt |
| 901 | ggctacctgt tgggccccac atcatgggaa aggctgtcaa ataatctttc ccaagctcca |
| 961 | aggcactcat ttgctccttc cccagcctcc aattagaaca agccacccac cagcctatct |
| 1021 | atcttccact gagagggacc tagcagaatg agagaagaca ttcatgtacc acctactagt |
| 1081 | ccctctctcc ccaacctctg ccagggcaat ctctaacttc aatcccgcct tcgacagtga |
| 1141 | aaaagctcta cttctacgct gacccaggga ggaaacacta ggaccctgtt gtatcctcaa |
| 1201 | ctgcaagttt ctggactagt ctcccaacgt ttgcctccca atgttgtccc tttccttcgt |
| 1261 | tcccatggta aagctcctct cgctttcctc ctgaggctac acccatgcgt ctctaggaac |
| 1321 | tggtcacaaa agtcatggtg cctgcatccc tgccaagccc ccctgaccct ctctccccac |
| 1381 | taccaccttc ttcctgagct gggggcacca gggagaatca gagatgctgg ggatgccaga |
| 1441 | gcaagactca aagaggcaga ggttttgttc tcaaatattt tttaataaat agacgaaacc |
| 1501 | acgaaaaaaa aaaaaaaaaa aaaaaaaaaa a |

MMP9 (SEQ ID NO: 6) - Homo sapiens matrix metallopeptidase 9
(MMP9), mRNA - NM_004994

| | |
|---|---|
| 1 | agacacctct gccctcacca tgagcctctg gcagcccctg gtcctggtgc tcctggtgct |
| 61 | gggctgctgc tttgctgccc ccagacagcg ccagtccacc cttgtgctct tccctggaga |
| 121 | cctgagaacc aatctcaccg acaggcagct ggcagaggaa tacctgtacc gctatggtta |
| 181 | cactcgggtg gcagagatgc gtggagagtc gaaatctctg gggcctgcgc tgctgcttct |
| 241 | ccagaagcaa ctgtccctgc ccgagaccgg tgagctggat agcgccacgc tgaaggccat |
| 301 | gcgaacccca cggtgcgggg tcccagacct gggcagattc caaacctttg agggcgacct |
| 361 | caagtggcac caccacaaca tcacctattg gatccaaaac tactcggaag acttgccgcg |
| 421 | ggcggtgatt gacgacgcct ttgcccgcgc cttcgcactg tggagcgcgg tgacgccgct |
| 481 | caccttcact cgcgtgtaca gccgggacgc agacatcgtc atccagtttg gtgtcgcgga |
| 541 | gcacggagac gggtatccct tcgacgggaa ggacgggctc ctggcacacg cctttcctcc |
| 601 | tggccccggc attcagggag acgcccattt cgacgatgac gagttgtggt ccctgggcaa |
| 661 | gggcgtcgtg gttccaactc ggtttggaaa cgcagatggc gcggcctgcc acttcccctt |
| 721 | catcttcgag ggccgctcct actctgcctg caccaccgac ggtcgctccg acggcttgcc |
| 781 | ctggtgcagt accacggcca actacgacac cgacgaccgg tttggcttct gccccagcga |
| 841 | gagactctac acccaggacg gcaatgctga tgggaaaccc tgccagtttc cattcatctt |
| 901 | ccaaggccaa tcctactccg cctgcaccac ggacggtcgc tccgacggct accgctggtg |
| 961 | cgccaccacc gccaactacg accgggacaa gctcttcggc ttctgcccga cccgagctga |
| 1021 | ctcgacggtg atggggggca actcggcggg ggagctgtgc gtcttcccct tcactttcct |
| 1081 | gggtaaggag tactcgacct gtaccagcga gggccgcgga gatgggcgcc tctggtgcgc |
| 1141 | taccacctcg aactttgaca gcgacaagaa gtggggcttc tgcccggacc aaggatacag |
| 1201 | tttgttcctc gtggcggcgc atgagttcgg ccacgcgctg ggcttagatc attcctcagt |
| 1261 | gccggaggcg ctcatgtacc ctatgtaccg cttcactgag gggcccccct tgcataagga |
| 1321 | cgacgtgaat ggcatccggc acctctatgg tcctcgccct gaacctgagc cacggcctcc |
| 1381 | aaccaccacc acaccgcagc ccacggctcc cccgacggtc tgccccaccg gaccccccac |
| 1441 | tgtccacccc tcagagcgcc ccacagctgg ccccacaggt cccccctcag ctggccccac |
| 1501 | aggtcccccc actgctggcc cttctacggc cactactgtg cctttgagtc cggtggacga |
| 1561 | tgcctgcaac gtgaacatct tcgacgccat cgcggagatt gggaaccagc tgtatttgtt |
| 1621 | caaggatggg aagtactggc gattctctga gggcaggggg agccggccgc agggcccctt |
| 1681 | ccttatcgcc gacaagtggc ccgcgctgcc ccgcaagctg gactcggtct ttgaggagcg |
| 1741 | gctctccaag aagcttttct tcttctctgg gcgccaggtg tgggtgtaca caggcgcgtc |
| 1801 | ggtgctgggc ccgaggcgtc tggacaagct gggcctggga gccgacgtgg cccaggtgac |
| 1861 | cggggccctc cggagtggca gggggaagat gctgctgttc agcgggcggc gcctctggag |
| 1921 | gttcgacgtg aaggcgcaga tggtggatcc ccggagcgcc agcgaggtgg accggatgtt |
| 1981 | ccccggggtg cctttggaca cgcacgacgt cttccagtac cgagagaaag cctatttctg |
| 2041 | ccaggaccgc ttctactggc gcgtgagttc ccggagtgag ttgaaccagg tggaccaagt |
| 2101 | gggctacgtg acctatgaca tcctgcagtg ccctgaggac tagggctccc gtcctgcttt |
| 2161 | ggcagtgcca tgtaaatccc cactgggacc aaccctgggg aaggagccag tttgccggat |
| 2221 | acaaactggt attctgttct ggaggaaagg gaggagtgga ggtgggctgg gccctctctt |
| 2281 | ctcacctttg ttttttgttg gagtgtttct aataaacttg gattctctaa cctttaaaaa |
| 2341 | aaaaaaaaaa aaaaaaaaaa aaaaaaaaaa aaaaaaaaaa aaaaaaa |

RETN (SEQ ID NO: 7) - Homo sapiens resistin, transcript variant 1
(RENT), mRNA - NM_020415

| | |
|---|---|
| 1 | gtgtgccgga tttggttagc tgagcccacc gagaggcgcc tgcaggatga aagctctctg |
| 61 | tctcctcctc ctccctgtcc tggggctgtt ggtgtctagc aagaccctgt gctccatgga |
| 121 | agaagccatc aatgagagga tccaggaggt cgccggctcc ctaatattta gggcaataag |
| 181 | cagcattggc ctggagtgcc agagcgtcac ctccaggggg gacctggcta cttgcccccg |
| 241 | aggcttcgcc gtcaccggct gcacttgtgg ctccgcctgt ggctcgtggg atgtgcgcgc |
| 301 | cgagaccaca tgtcactgcc agtgcgcggg catggactgg accggagcgc gctgctgtcg |
| 361 | tgtgcagccc tgaggtcgcg cgcagcgcgt gcacagcgcg ggcggaggcg gctccaggtc |
| 421 | cggaggggtt gcgggggagc tggaaataaa cctggagatg atgatgatga tgatgatg |

TDRD9 (SEQ ID NO: 8) - Homo sapiens tudor domain containing 9
(TDRD9), mRNA - NM_153046

| | |
|---|---|
| 1 | ttgggggatg ccgacgcctg ggccttgagg atgctgcgga agctcaccat cgagcagatc |
| 61 | aacgactggt tcaccatcgg caagacggtg accaatgtgg agctgctggg cgcgccgccc |
| 121 | gccttcccgg caggggcggc cagggaggag gtgcagcgcc aggacgtggc ccccggcgct |
| 181 | ggtcccgcgg cccaggctcc ggctctggcc caagctccgg cccggccggc cgctgcgttc |
| 241 | gaaaggtcac tcagccaaag gagctcagaa gtagagtata ttaacaaata cagacagctc |
| 301 | gaagcacaag agcttgatgt gtgtcgcagt gtccaaccaa ccagtgggcc aggtccaagg |
| 361 | ccatctttgg ctaaattaag cagtgtgacg tgcatcccag ggacaactta taaatatcct |
| 421 | gatttgccta taagtcgata caaggaagag gttgtgtctt tgatagaaag taattccgtg |
| 481 | gtgattatcc atggggccac gggaagcggt aaaagcactc agctcccgca gtatatcttg |
| 541 | gaccactacg ttcagcgctc cgcctactgc agcattgtgg tcacccagcc ccggaagata |
| 601 | ggggcaagca gcatcgccag gtggatcagt aaagagcgtg cctggaccct gggaggtgtg |
| 661 | gtgggctacc aggtagggct agagaaaata gcaacagagg acaccaggct aatttatatg |
| 721 | acaactggag tcctgcttca gaaaatagtt agtgccaaga gtttgatgga attcacacat |
| 781 | atcatcattg atgaagtaca cgaacgaaca gaagaaatgg atttcctgct attggtagtc |
| 841 | cgcaaactct taagaacaaa ttcacgtttt gtgaaggtgg tcctgatgtc ggctaccatc |
| 901 | agctgtaaag agtttgcaga ctactttgct gttcctgttc aaaacaagat gaatcctgca |
| 961 | tatatttttg aagtggaagg caagccccat tcagttgaag agtattatct taatgatttg |
| 1021 | gagcacattc atcatagcaa gctctctcct catctcctgg aggaaccggt gataactaag |
| 1081 | gatatatatg aagttgctgt ctctctcatt cagatgtttg atgacttgga tatgaaggag |
| 1141 | agtgggaaca aggcttggtc gggggcccag tttgtgttgg agcgaagcag tgtgttggtg |
| 1201 | tttttgccag gtctgggtga aataaattat atgcatgaac ttctcacaag cctggttcat |
| 1261 | aaaaggttgc aggtctatcc actccattca agtgtggctt tagaagaaca gaataatgtc |
| 1321 | tttttaagtc cagtccctgg gtacagaaag attattctgt ccaccaatat tgcagagagt |
| 1381 | tctgtcacag ttccagatgt caaatatgtt atagattttt gtttgactag aactttggtc |
| 1441 | tgtgatgaag atacaaatta tcagagtctg cgattgagtt gggcttctaa aaccagctgt |
| 1501 | aatcagagaa aaggccgtgc tggacgagtg tctagagggt actgttaccg gctggtacac |
| 1561 | aaggatttct gggacaactc catccctgat catgttgttc ctgagatgtt gcgttgtcca |
| 1621 | ttaggaagca cgatcttgaa agtgaaatta cttgacatgg gtgagccgag agctctgctg |
| 1681 | gccactgccc tttccccgcc tggtctgagt gacattgagc gcaccatcct tctactaaag |
| 1741 | gaggttggag cacttgcagt gagtgggcag agagaagatg aaaaccccca tgatggtgaa |
| 1801 | ttgaccttct taggaagagt tttagcccaa cttcctgtaa atcagcaact tggtaaactc |
| 1861 | atagtccttg gacatgtatt tggatgtcta gatgaatgtc ttattatagc ggcagctctt |
| 1921 | tctttgaaga atttttttgc aatgcctttc cggcagcatc tcgatggata taggaacaaa |
| 1981 | gtgaatttct ctggcagtag caagagtgac tgtattgcac ttgttgaggc atttaaaaca |
| 2041 | tggaaggctt gcagacagac aggggagctg cggtacccga aggatgaact taattgggga |
| 2101 | cggttaaatt acattcaaat caagagaatt agagaggtgg ctgaattata tgaagaattg |
| 2161 | aagactagaa tctcacagtt caacatgcat gttgattctc ggcgacctgt catggaccaa |
| 2221 | gagtatatat ataagcagcg attcatccta caggttgtat tggcaggtgc tttctatcca |
| 2281 | aattacttta cttttggaca gccggatgag gagatggcgg tgagggagct ggctggcaag |
| 2341 | gaccccaaga caactgtcgt gttgaaacac attcctccct atggatttct ttactataaa |
| 2401 | caactacagt ctctctttag acagtgtggt caagtcaaat ccattgtatt tgatggtgca |
| 2461 | aaagcctttg tggaattctc acgaaatcca acagagagat ttaaaaccct tcctgcagta |
| 2521 | tatatggcaa ttaagatgtc tcaactaaaa gtttcacttg aactcagcgt tcattctgca |
| 2581 | gaggaaattg aagggaaggt gcaaggcatg aacgtctcaa agctcaggaa cacaagggtg |
| 2641 | aatgtggact tccagaagca gacggtagat cctatgcaag tctcctttaa cacatcagac |
| 2701 | aggtcccaga cagttacaga tctccttcta actattgatg tcacagaggt ggttgaagtg |
| 2761 | ggacactttt ggggatacag gattgatgaa aacaactcag agattctgaa aaagcttact |
| 2821 | gctgaaatca accaactgac gctggtgccc ttgcccactc acccacatcc agacttggtc |
| 2881 | tgtctggcac cttttgctga ttttgataaa caacgctact ttagagctca agtcctttat |
| 2941 | gtttctggaa attctgctga ggtattcttt gtagattatg gcaataagtc tcatgtagat |
| 3001 | ctacatcttt tgatggagat tccctgtcaa tttcttgaac ttcctttcca ggctttggaa |
| 3061 | tttaagattt gcaaaatgag accatcagca aagtctcttg tttgtggcaa gcactggagt |
| 3121 | gacggggcca gccagtggtt cgcctctctg gtgagcggct gcaccctcct tgtgaaggtc |
| 3181 | ttctctgtgg tgcacagcgt cctgcacgtg gatgtgtacc agtactcagg ggtccaggat |
| 3241 | gccatcaaca taagagacgt cctcatccag cagggctatg ccgagctcac ggaggagtcc |
| 3301 | tacgagtcca agcaaagcca tgaagttctc aagggcctct tttccaagtc agtagaaaac |
| 3361 | atgacagatg gctctgtgcc ctttcccatg aaagacgacg agaaatatct catccggatt |
| 3421 | ttgttagaga gcttttctac caataaactg ggtactccaa actgtaaggc agaacttcac |
| 3481 | gggcctttta acccttatga actaaagtgc catagtttga ccagaatatc caaattcagg |
| 3541 | tgtgtttgga ttgagaagga gagcatcaac tctgtcatta tcagtgacgc ccctgaagac |
| 3601 | cttcaccaga gaatgctggt tgcagcttcc ctttccatca atgcgactgg atctacgatg |
| 3661 | ctgctgagag aaacctctct gatgcctcat atccctggcc tcccggctct cctcagcatg |
| 3721 | ttattcgcac cggtgataga gttaaggatt gatcagaatg gcaagtacta tactggagtc |
| 3781 | ctttgtggtt tggggtggaa tccagctaca ggggcttcca tactgcccga gcacgacatg |
| 3841 | gagcttgcgt ttgacgttca attcagcgtg gaggatgtcg tcgaggttaa tattctcagg |
| 3901 | gctgctatta acaagctagt ctgtgatgga ccaaatggat gcaagtgtct tgggccagag |
| 3961 | agagttgcgc agcttcaaga cattgcccgt cagaagcttt taggtttgtt ctgtcagtca |
| 4021 | aaaccaaggg agaagattgt tcccaagtgg catgaaaagc cctacgagtg gaatcaggtt |
| 4081 | gatccaaagc tggtcatgga gcaggccgac cgtgagagca gcagagggaa gaacaccttt |
| 4141 | ctctaccagc tccacaaact ggttgtgctc ggcacctgag catgtccaca ggtggcctcc |
| 4201 | agcacacccc tcaggaagct gtggaggctg gattccaggc tccctccgca gactgacttt |
| 4261 | cctctgtgtc tgggtgttac agtctgtgcc cactgcatcc taaaggcctt ttctttcttc |
| 4321 | ttttctcttt gggtgatagt cagagagtgg tgtttttgtt caggtgggaa ggattggaaa |
| 4381 | ctctagtctt ttctagaaac agaaaatcac tgtattaaat attttggaaa gattgttctg |
| 4441 | aaagaagtct gtttggataa agagctgtat tttgctttaa atttattaag gtaaatataa |
| 4501 | gtagttaatc ttagatgtaa ggttccagaa tgtgcttaca tattctgttc tgttacagtg |
| 4561 | atttaaacca gtagtatagg aaaaaactta aaaaacaaaa aaaccatgta gtattttctg |
| 4621 | attttttttt ccatgaggga aaatatctaa tttttataag actaagttga gttatacttc |
| 4681 | ttggttcaca ttttggaaat cagagattac agattacatg gccatagctt atctgtgtta |
| 4741 | aaacaataaa agcattaaat gaaaaaaaaa aaaaaaaaaa aa |

ITGA7 (SEQ ID NO: 9) - Homo sapiens integrin subunit alpha 7
(ITGA7), transcript variant 2, mRNA - NM_002206

| | |
|---|---|
| 1 | gggcgccgga gctgcggctg ctgtagttgt cctagccggt gctggggcgg cggggtggcg |
| 61 | gagcggcggg cgggcgggag ggctggcggg gcgaacgtct gggagacgtc tgaaagacca |
| 121 | acgagacttt ggagaccaga gacgcgcctg gggggacctg gggcttgggg cgtgcgagat |
| 181 | ttcccttgca ttcgctggga gctcgcgcag ggatcgtccc atggccgggg ctcggagccg |
| 241 | cgacccttgg ggggcctccg ggatttgcta cctttttggc tccctgctcg tcgaactgct |
| 301 | cttctcacgg gctgtcgcct tcaatctgga cgtgatgggt gccttgcgca aggagggcga |
| 361 | gccaggcagc ctcttcggct tctctgtggc cctgcaccgg cagttgcagc cccgacccca |
| 421 | gagctggctg ctggtgggtg ctccccaggc cctggctctt cctgggcagc aggcgaatcg |
| 481 | cactggaggc ctcttcgctt gcccgttgag cctggaggag actgactgct acagagtgga |
| 541 | catcgaccag ggagctgata tgcaaaagga aagcaaggag aaccagtggt tgggagtcag |
| 601 | tgttcggagc caggggcctg ggggcaagat tgttacctgt gcacaccgat atgaggcaag |
| 661 | gcagcgagtg gaccagatcc tggagacgcg ggatatgatt ggtcgctgct ttgtgctcag |
| 721 | ccaggacctg gccatccggg atgagttgga tggtggggaa tggaagttct gtgagggacg |
| 781 | cccccaaggc catgaacaat ttgggttctg ccagcagggc acagctgccg ccttctcccc |
| 841 | tgatagccac tacctcctct ttggggcccc aggaacctat aattggaagg ggttgctttt |
| 901 | tgtgaccaac attgatagct cagaccccga ccagctggtg tataaaactt tggaccctgc |
| 961 | tgaccggctc ccaggaccag ccggagactt ggccctcaat agctacttag gcttctctat |
| 1021 | tgactcgggg aaaggtctgg tgcgtgcaga agagctgagc tttgtggctg gagccccccg |
| 1081 | cgccaaccac aagggtgctg tggtcatcct gcgcaaggac agcgccagtc gcctggtgcc |
| 1141 | cgaggttatg ctgtctgggg agcgcctgac ctccggcttt ggctactcac tggctgtggc |
| 1201 | tgacctcaac agtgatggct ggccagacct gatagtgggt gccccctact tctttgagcg |
| 1261 | ccaagaagag ctggggggtg ctgtgtatgt gtacttgaac caggggggtc actgggctgg |
| 1321 | gatctcccct ctccggctct gcggctcccc tgactccatg ttcgggatca gcctggctgt |
| 1381 | cctgggggac ctcaaccaag atggctttcc agatattgca gtgggtgccc cctttgatgg |
| 1441 | tgatgggaaa gtcttcatct accatgggag cagcctgggg gttgtcgcca aaccttcaca |
| 1501 | ggtgctggag ggcgaggctg tgggcatcaa gagcttcggc tactccctgt caggcagctt |
| 1561 | ggatatggat gggaaccaat accctgacct gctggtgggc tccctggctg acaccgcagt |
| 1621 | gctcttcagg gccagaccca tcctccatgt ctcccatgag gtctctattg ctccacgaag |
| 1681 | catcgacctg gagcagccca actgtgctgg cggccactcg gtctgtgtgg acctaagggt |
| 1741 | ctgtttcagc tacattgcag tccccagcag ctatagccct actgtggccc tggactatgt |
| 1801 | gttagatgcg gacacagacc ggaggctccg gggccaggtt ccccgtgtga cgttcctgag |
| 1861 | ccgtaacctg gaagaaccca agcaccaggc ctcgggcacc gtgtggctga agcaccagca |
| 1921 | tgaccgagtc tgtggagacg ccatgttcca gctccaggaa aatgtcaaag acaagcttcg |
| 1981 | ggccattgta gtgaccttgt cctacagtct ccagacccct cggctccggc gacaggctcc |
| 2041 | tggccagggg ctgcctccag tggcccccat cctcaatgcc caccagccca gcacccagcg |
| 2101 | ggcagagatc cacttcctga agcaaggctg tggtgaagac aagatctgcc agagcaatct |
| 2161 | gcagctggtc cgcgcccgct tctgtacccg ggtcagcgac acggaattcc aacctctgcc |
| 2221 | catggatgtg gatggaacaa cagccctgtt tgcactgagt gggcagccag tcattggcct |
| 2281 | ggagctgatg gtcaccaacc tgccatcgga cccagcccag ccccaggctg atggggatga |
| 2341 | tgcccatgaa gcccagctcc tggtcatgct tcctgactca ctgcactact caggggtccg |
| 2401 | ggccctggac cctgcggaga agccactctg cctgtccaat gagaatgcct cccatgttga |
| 2461 | gtgtgagctg gggaacccca tgaagagagg tgcccaggtc accttctacc tcatccttag |
| 2521 | cacctccggg atcagcattg agaccacgga actggaggta gagctgctgt tggccacgat |
| 2581 | cagtgagcag gagctgcatc cagtctctgc acgagcccgt gtcttcattg agctgccact |
| 2641 | gtccattgca ggaatggcca ttccccagca actcttcttc tctggtgtgg tgaggggcga |
| 2701 | gagagccatg cagtctgagc gggatgtggg cagcaaggtc aagtatgagg tcacggtttc |
| 2761 | caaccaaggc cagtcgctca gaaccctggg ctctgccttc ctcaacatca tgtggcctca |
| 2821 | tgagattgcc aatgggaagt ggttgctgta cccaatgcag gttgagctgg agggcgggca |
| 2881 | ggggcctggg cagaaagggc tttgctctcc caggcccaac atcctccacc tggatgtgga |
| 2941 | cagtagggat aggaggcggc gggagctgga gccacctgag cagcaggagc ctggtgagcg |
| 3001 | gcaggagccc agcatgtcct ggtggccagt gtcctctgct gagaagaaga aaaacatcac |
| 3061 | cctggactgc gcccggggca cggccaactg tgtggtgttc agctgcccac tctacagctt |
| 3121 | tgaccgcgcg gctgtgctgc atgtctgggg ccgtctctgg aacagcacct ttctggagga |
| 3181 | gtactcagct gtgaagtccc tggaagtgat tgtccgggcc aacatcacag tgaagtcctc |
| 3241 | cataaagaac ttgatgctcc gagatgcctc cacagtgatc ccagtgatgg tatacttgga |
| 3301 | ccccatggct gtggtggcag aaggagtgcc ctggtgggtc atcctcctgg ctgtactggc |
| 3361 | tgggctgctg gtgctagcac tgctggtgct gctcctgtgg aagatgggat tcttcaaacg |
| 3421 | ggcgaagcac cccgaggcca ccgtgcccca gtaccatgcg gtgaagattc ctcgggaaga |
| 3481 | ccgacagcag ttcaaggagg agaagacggg caccatcctg aggaacaact ggggcagccc |
| 3541 | ccggcgggag ggcccggatg cacaccccat cctggctgct gacgggcatc ccgagctggg |
| 3601 | ccccgatggg catccagggc caggcaccgc ctaggttccc atgtcccagc ctggcctgtg |
| 3661 | gctgccctcc atcccttccc cagagatggc tccttgggat gaagagggta gagtgggctg |
| 3721 | ctggtgtcgc atcaagattt ggcaggatcg gcttcctcag gggcacagac ctctcccacc |
| 3781 | cacaagaact cctcccaccc aacttcccct tagagtgctg tgagatgaga gtgggtaaat |
| 3841 | cagggacagg gccatggggt agggtgagaa gggcaggggt gtcctgatgc aaaggtgggg |
| 3901 | agaagggatc ctaatccctt cctctcccat tcaccctgtg taacaggacc ccaaggacct |
| 3961 | gcctccccgg aagtgcctta acctagaggg tcggggagga ggttgtgtca ctgactcagg |
| 4021 | ctgctccttc tctagtttcc cctctcatct gaccttagtt tgctgccatc agtctagtgg |
| 4081 | tttcgtggtt tcgtctattt attaaaaaat atttgagaac aaaaaaaaaa aaaaaaaa |

BMX (SEQ ID NO: 10) - Homo sapiens BMX non-receptor tyrosine
kinase (BMX), transcript variant 2, mRNA - NM_001721

| | |
|---|---|
| 1 | gggaatatga gtgatggtgc ctcaaagcag taactttttg cttagagctt gagagtcaaa |
| 61 | gttaaggacc cacatgtata cttcggctct agcgagtcta aggatgataa tatggataca |
| 121 | aaatctattc tagaagaact tcttctcaaa agatcacagc aaaagaagaa aatgtcacca |
| 181 | aataattaca aagaacggct ttttgttttg accaaaacaa acctttccta ctatgaatat |
| 241 | gacaaaatga aaaggggcag cagaaaagga tccattgaaa ttaagaaaat cagatgtgtg |
| 301 | gagaaagtaa atctcgagga gcagacgcct gtagagagac agtacccatt tcagattgtc |
| 361 | tataaagatg ggcttctcta tgtctatgca tcaaatgaag agagccgaag tcagtggttg |
| 421 | aaagcattac aaaaagagat aaggggtaac ccccacctgc tggtcaagta ccatagtggg |
| 481 | ttcttcgtgg acgggaagtt cctgtgttgc cagcagagct gtaaagcagc cccaggatgt |
| 541 | accctctggg aagcatatgc taatctgcat actgcagtca atgaagagaa acacagagtt |
| 601 | cccaccttcc cagacagagt gctgaagata cctcgggcag ttcctgttct caaaatggat |
| 661 | gcaccatctt caagtaccac tctagcccaa tatgacaacg aatcaaagaa aaactatggc |
| 721 | tcccagccac catcttcaag taccagtcta gcgcaatatg acagcaactc aaagaaaatc |
| 781 | tatggctccc agccaaactt caacatgcag tatattccaa gggaagactt ccctgactgg |
| 841 | tggcaagtaa gaaaactgaa aagtagcagc agcagtgaag atgttgcaag cagtaaccaa |
| 901 | aaagaaagaa atgtgaatca caccacctca aagatttcat gggaattccc tgagtcaagt |
| 961 | tcatctgaag aagaggaaaa cctggatgat tatgactggt ttgctggtaa catctccaga |
| 1021 | tcacaatctg aacagttact cagacaaaag ggaaaagaag gagcatttat ggttagaaat |
| 1081 | tcgagccaag tgggaatgta cacagtgtcc ttatttagta aggctgtgaa tgataaaaaa |
| 1141 | ggaactgtca aacattacca cgtgcataca aatgctgaga acaaattata cctggcagaa |
| 1201 | aactactgtt ttgattccat tccaaagctt attcattatc atcaacacaa ttcagcaggc |
| 1261 | atgatcacac ggctccgcca ccctgtgtca acaaaggcca acaaggtccc cgactctgtg |
| 1321 | tccctgggaa atggaatctg ggaactgaaa agagaagaga ttaccttgtt gaaggagctg |
| 1381 | ggaagtggcc agtttggagt ggtccagctg ggcaagtgga aggggcagta tgatgttgct |
| 1441 | gttaagatga tcaaggaggg ctccatgtca gaagatgaat tctttcagga ggcccagact |
| 1501 | atgatgaaac tcagccatcc caagctggtt aaattctatg gagtgtgttc aaaggaatac |
| 1561 | cccatataca tagtgactga atatataagc aatggctgct tgctgaatta cctgaggagt |
| 1621 | cacggaaaag gacttgaacc ttcccagctc ttagaaatgt gctacgatgt ctgtgaaggc |
| 1681 | atggccttct tggagagtca ccaattcata caccgggact tggctgctcg taactgcttg |
| 1741 | gtggacagag atctctgtgt gaaagtatct gactttggaa tgacaaggta tgttcttgat |
| 1801 | gaccagtatg tcagttcagt cggaacaaag tttccagtca agtggtcagc tccagaggtg |
| 1861 | tttcattact tcaaatacag cagcaagtca gacgtatggg catttgggat cctgatgtgg |
| 1921 | gaggtgttca gcctggggaa gcagccctat gacttgtatg acaactccca ggtggttctg |
| 1981 | aaggtctccc agggccacag gctttaccgg ccccacctgg catcggacac catctaccag |
| 2041 | atcatgtaca gctgctggca cgagcttcca gaaaagcgtc ccacatttca gcaactcctg |
| 2101 | tcttccattg aaccacttcg ggaaaaagac aagcattgaa gaagaaatta ggagtgctga |
| 2161 | taagaatgaa tatagatgct ggccagcatt ttcattcatt ttaaggaaag tagcaaggca |
| 2221 | taatgtaatt tagctagttt ttaatagtgt tctctgtatt gtctattatt tagaaatgaa |
| 2281 | caaggcagga aacaaaagat tcccttgaaa tttagatcaa attagtaatt ttgtttatgc |
| 2341 | tgctcctgat ataacacttt ccagcctata gcagaagcac attttcagac tgcaatatag |
| 2401 | agactgtgtt catgtgtaaa gactgagcag aactgaaaaa ttacttattg gatattcatt |
| 2461 | cttttcttta tattgtcatt gtcacaacaa ttaaatatac taccaagtac agaaatgtgg |
| 2521 | aaaaaaaaaa |

HP (SEQ ID NO: 11) - Homo sapiens haptoglobin (HP), transcript
variant 1, mRNA - NM_005143

| | |
|---|---|
| 1 | agcataaaaa gaccagcaga tgccccacag cactgctctt ccagaggcaa gaccaaccaa |
| 61 | gatgagtgcc ctgggagctg tcattgccct cctgctctgg ggacagcttt ttgcagtgga |
| 121 | ctcaggcaat gatgtcacgg atatcgcaga tgacggctgc ccgaagcccc ccgagattgc |
| 181 | acatggctat gtggagcact cggttcgcta ccagtgtaag aactactaca aactgcgcac |
| 241 | agaaggagat ggagtataca ccttaaatga taagaagcag tggataaata aggctgttgg |
| 301 | agataaactt cctgaatgtg aagcagatga cggctgcccg aagccccccg agattgcaca |
| 361 | tggctatgtg gagcactcgg ttcgctacca gtgtaagaac tactacaaac tgcgcacaga |
| 421 | aggagatgga gtgtacacct taaacaatga gaagcagtgg ataaataagg ctgttggaga |
| 481 | taaacttcct gaatgtgaag cagtatgtgg gaagcccaag aatccggcaa acccagtgca |
| 541 | gcggatcctg ggtggacacc tggatgccaa aggcagcttt ccctggcagg ctaagatggt |
| 601 | ttcccaccat aatctcacca caggtgccac gctgatcaat gaacaatggc tgctgaccac |
| 661 | ggctaaaaat ctcttcctga accattcaga aaatgcaaca gcgaaagaca ttgcccctac |
| 721 | tttaacactc tatgtgggga aaaagcagct tgtagagatt gagaaggttg ttctacaccc |
| 781 | taactactcc caggtagata ttgggctcat caaactcaaa cagaaggtgt ctgttaatga |
| 841 | gagagtgatg cccatctgcc taccttcaaa ggattatgca gaagtagggc gtgtgggtta |
| 901 | tgtttctggc tgggggcgaa atgccaattt taaatttact gaccatctga agtatgtcat |
| 961 | gctgcctgtg gctgaccaag accaatgcat aaggcattat gaaggcagca cagtccccga |
| 1021 | aaagaagaca ccgaagagcc ctgtaggggt gcagcccata ctgaatgaac acaccttctg |
| 1081 | tgctggcatg tctaagtacc aagaagacac ctgctatggc gatgcgggca gtgcctttgc |
| 1141 | cgttcacgac ctggaggagg acacctggta tgcgactggg atcttaagct ttgataagag |
| 1201 | ctgtgctgtg gctgagtatg gtgtgtatgt gaaggtgact tccatccagg actgggttca |
| 1261 | gaagaccata gctgagaact aatgcaaggc tggccggaag cccttgcctg aaagcaagat |
| 1321 | ttcagcctgg aagagggcaa agtggacggg agtggacagg agtggatgcg ataagatgtg |
| 1381 | gtttgaagct gatgggtgcc agccctgcat tgctgagtca atcaataaag agctttcttt |
| 1441 | tgacccataa aaaaaaaaaa aaaaaaaaaa aaaaaaaa |

IGFBP2 (SEQ ID NO: 12) - Homo sapiens insulin like growth factor
binding protein 2, 36 kDa (IGFBP2), transcript variant 1, mRNA -
NM_000597

| | |
|---|---|
| 1 | tgcggcggcg agggaggagg aagaagcgga ggaggcggct cccgcgctcg cagggccgtg |
| 61 | ccacctgccc gcccgcccgc tcgctcgctc gcccgccgcg ccgcgctgcc gaccgccagc |
| 121 | atgctgccga gagtgggctg ccccgcgctg ccgctgccgc cgccgccgct gctgccgctg |
| 181 | ctgccgctgc tgctgctgct actgggcgcg agtggcggcg gcggcggggc gcgcgcggag |
| 241 | gtgctgttcc gctgcccgcc ctgcacaccc gagcgcctgg ccgcctgcgg gcccccgccg |
| 301 | gttgcgccgc ccgccgcggt ggccgcagtg gccggaggcg cccgcatgcc atgcgcggag |
| 361 | ctcgtccggg agccgggctg cggctgctgc tcggtgtgcg cccggctgga gggcgaggcg |
| 421 | tgcggcgtct acaccccgcg ctgcggccag gggctgcgct gctatcccca cccgggctcc |
| 481 | gagctgcccc tgcaggcgct ggtcatgggc gagggcactt gtgagaagcg ccgggacgcc |
| 541 | gagtatggcg ccagcccgga gcaggttgca gacaatggcg atgaccactc agaaggaggc |
| 601 | ctggtggaga accacgtgga cagcaccatg aacatgttgg gcgggggagg cagtgctggc |
| 661 | cggaagcccc tcaagtcggg tatgaaggag ctggccgtgt tccgggagaa ggtcactgag |
| 721 | cagcaccggc agatgggcaa gggtggcaag catcaccttg gcctggagga gcccaagaag |
| 781 | ctgcgaccac cccctgccag gactccctgc caacaggaac tggaccaggt cctggagcgg |
| 841 | atctccacca tgcgccttcc ggatgagcgg ggccctctgg agcacctcta ctccctgcac |
| 901 | atccccaact gtgacaagca tggcctgtac aacctcaaac agtgcaagat gtctctgaac |
| 961 | gggcagcgtg gggagtgctg gtgtgtgaac cccaacaccg ggaagctgat ccagggagcc |
| 1021 | cccaccatcc ggggggaccc cgagtgtcat ctcttctaca atgagcagca ggaggctcgc |
| 1081 | ggggtgcaca cccagcggat gcagtagacc gcagccagcc ggtgcctggc gcccctgccc |
| 1141 | cccgcccctc tccaaacacc ggcagaaaac ggagagtgct tgggtggtgg gtgctggagg |
| 1201 | attttccagt tctgacacac gtatttatat ttggaaagag accagcaccg agctcggcac |
| 1261 | ctccccggcc tctctcttcc cagctgcaga tgccacacct gctccttctt gctttccccg |
| 1321 | ggggaggaag ggggttgtgg tcggggagct ggggtacagg tttggggagg gggaagagaa |
| 1381 | atttttattt ttgaacccct gtgtcccttt tgcataagat taaaggaagg aaaagtaaa |

ALPL (SEQ ID NO: 13) - Homo sapiens alkaline phosphatase,
liver/bone/kidney (ALPL), transcript variant 1, mRNA - NM_000478

| | |
|---|---|
| 1 | gggctgcccg ggcctcactc gggccccgcg gccgccttta taaggcggcg ggggtggtgg |
| 61 | cccgggccgc gttgcgctcc cgccactccg cgcccgctat cctggctccg tgctcccacg |
| 121 | cgcttgtgcc tggacggacc ctcgccagtg ctctgcgcag gattggaaca tcagttaaca |
| 181 | tctgaccact gccagcccac cccctcccac ccacgtcgat tgcatctctg ggctccaggg |
| 241 | ataaagcagg tcttggggtg caccatgatt tcaccattct tagtactggc cattggcacc |
| 301 | tgccttacta actccttagt gccagagaaa gagaaagacc ccaagtactg gcgagaccaa |
| 361 | gcgcaagaga cactgaaata tgccctggag cttcagaagc tcaacaccaa cgtggctaag |
| 421 | aatgtcatca tgttcctggg agatgggatg ggtgtctcca cagtgacggc tgcccgcatc |
| 481 | ctcaagggtc agctccacca caaccctggg gaggagacca ggctggagat ggacaagttc |
| 541 | cccttcgtgg ccctctccaa gacgtacaac accaatgccc aggtccctga cagcgccggc |
| 601 | accgccaccg cctacctgtg tggggtgaag gccaatgagg gcaccgtggg ggtaagcgca |
| 661 | gccactgagc gttcccggtg caacaccacc caggggaacg aggtcacctc catcctgcgc |
| 721 | tgggccaagg acgctgggaa atctgtgggc attgtgacca ccacgagagt gaaccatgcc |
| 781 | acccccagcg ccgcctacgc ccactcggct gaccgggact ggtactcaga caacgagatg |
| 841 | ccccctgagg ccttgagcca gggctgtaag gacatcgcct accagctcat gcataacatc |
| 901 | agggacattg acgtgatcat ggggggtggc cggaaataca tgtaccccaa gaataaaact |
| 961 | gatgtggagt atgagagtga cgagaaagcc aggggcacga ggctggacgg cctggacctc |
| 1021 | gttgacacct ggaagagctt caaaccgaga tacaagcact cccacttcat ctggaaccgc |
| 1081 | acggaactcc tgacccttga cccccacaat gtggactacc tattgggtct cttcgagcca |
| 1141 | ggggacatgc agtacgagct gaacaggaac aacgtgacgg acccgtcact ctccgagatg |
| 1201 | gtggtggtgg ccatccagat cctgcggaag aaccccaaag gcttcttctt gctggtggaa |
| 1261 | ggaggcagaa ttgaccacgg gcaccatgaa ggaaaagcca agcaggccct gcatgaggcg |
| 1321 | gtggagatgg accgggccat cgggcaggca ggcagcttga cctcctcgga agacactctg |
| 1381 | accgtggtca ctgcggacca ttcccacgtc ttcacatttg gtggatacac cccccgtggc |
| 1441 | aactctatct ttggtctggc ccccatgctg agtgacacag acaagaagcc cttcactgcc |
| 1501 | atcctgtatg gcaatgggcc tggctacaag gtggtgggcg gtgaacgaga gaatgtctcc |
| 1561 | atggtggact atgctcacaa caactaccag gcgcagtctg ctgtgcccct gcgccacgag |
| 1621 | acccacggcg gggaggacgt ggccgtcttc tccaagggcc ccatggcgca cctgctgcac |
| 1681 | ggcgtccacg agcagaacta cgtcccccac gtgatggcgt atgcagcctg catcggggcc |
| 1741 | aacctcggcc actgtgctcc tgccagctcg gcaggcagcc ttgctgcagg ccccctgctg |
| 1801 | ctcgcgctgg ccctctaccc cctgagcgtc ctgttctgag ggcccagggc ccgggcaccc |
| 1861 | acaagcccgt gacagatgcc aacttcccac acggcagccc ccccctcaag gggcagggag |
| 1921 | gtgggggcct cctcagcctc tgcaactgca agaaagggga cccaagaaac caaagtctgc |
| 1981 | cgcccacctc gctcccctct ggaatcttcc ccaagggcca aacccacttc tggcctccag |
| 2041 | cctttgctcc ctccccgctg ccctttggcc aacagggtag atttctcttg ggcaggcaga |
| 2101 | gagtacagac tgcagacatt ctcaaagcct cttatttttc tagcgaacgt atttctccag |
| 2161 | acccagaggc cctgaagcct ccgtggaaca ttctggatct gaccctccca gtctcatctc |
| 2221 | ctgaccctcc cactcccatc tccttacctc tggaaccccc caggccctac aatgctcatg |
| 2281 | tccctgtccc caggcccagc cctccttcag gggagttgag gtctttctcc tcaggacaag |
| 2341 | gccttgctca ctcactcact ccaagaccac cagggtccca ggaagccggt gcctgggtgg |
| 2401 | ccatcctacc cagcgtggcc caggccggga agagccacct ggcagggctc acactcctgg |
| 2461 | gctctgaaca cacacgccag ctcctctctg aagcgactct cctgtttgga acggcaaaaa |
| 2521 | aaaatttttt tttctctttt tggtggtggt taaaagggaa cacaaaacat ttaaataaaa |
| 2581 | ctttccaaat atttccgagg acaaaaaaaa aaa |

DACH1 (SEQ ID NO: 14) - Homo sapiens dachshund family transcription
factor 1 (DACH1), transcript variant 1, mRNA - NM_005143

| | |
|---|---|
| 1 | atctttgatc aatgtacttg ccagggagag cccaagtcct tcaaacctcc tccttttcac |
| 61 | cttcatcctt aactttgtgc tagagcgaga cccacacaac aacagccgac cctccccgcc |
| 121 | ccacccccac ccccaaacca gccctcgatc ccagcccccg gagaggactc gcatttcgac |
| 181 | ttgcgggaca cttttgtgcg ttcctctcca gagcgcctct cgtgctcgcc cctcttgcgc |
| 241 | tcgctcttta ttaccttcac ctccttttct cccccttctc tccctttctc cttctcgttc |
| 301 | tctcccggag ttgttgttgc ccccctcgct ccttctcccc ccttttttcc ccttcccctc |
| 361 | ccgggggtgt gtggcaactt ttcctctcgc ttctcctccg tctgtttccc cttatatgtg |
| 421 | accatggcag tgccggcggc tttgatccct ccgacccagc tggtcccccc tcaaccccca |
| 481 | atctccacgt ctgcttcctc ctctggcacc accacctcca cctcttcggc gacttcgtct |
| 541 | ccggctcctt ccatcggacc cccggcgtcc tctgggccaa ctctgttccg cccggagccc |
| 601 | atcgcttcgg cggcggcggc ggcggccaca gtcacctcta ccggcggcgg cggcggcggc |
| 661 | ggcggcagcg gaggcggcgg cggcagcagc ggcaacggag gcggcggtgg cggcggcggc |
| 721 | ggtggcagca actgcaaccc caacctggcg gccgcgagca acggcagcgg cggcggcggc |
| 781 | ggcggcatca gcgctggcgg cggcgtcgct tccagcaccc ccatcaacgc cagcaccggc |
| 841 | agcagcagca gcagcagtag cagcagcagc agcagcagca gtagtagcag cagcagcagt |
| 901 | agcagcagca gctgcggccc cctccccggg aaacccgtgt actcaacccc gtccccagtg |
| 961 | gaaaacaccc ctcagaataa tgagtgcaaa atggtggatc tgaggggggc caaagtggct |
| 1021 | tccttcacgg tggagggctg cgagctgatc tgcctgcccc aggctttcga cctgttcctg |
| 1081 | aagcacttgg tggggggctt gcatacggtc tacaccaagc tgaagcggct ggagatcacg |
| 1141 | ccggtggtgt gcaatgtgga acaagttcgc atcctgaggg gactgggcgc catccagcca |
| 1201 | ggagtgaacc gctgcaaact catctccagg aaggacttcg agaccctcta caatgactgc |
| 1261 | accaacgcaa gttctagacc tggaaggcct cctaagagga ctcaaagtgt cacctcccca |
| 1321 | gagaactctc acatcatgcc gcattctgtc cctggtctca tgtctcctgg gataattcca |
| 1381 | ccaacaggtc tgacagcagc cgctgcagca gctgctgctg ctaccaatgc agctattgct |
| 1441 | gaagcaatga aggtgaaaaa aatcaaatta gaagccatga gcaactatca tgccagtaat |
| 1501 | aaccaacatg gagcagactc tgaaaacggg gacatgaatt caagtgtcgg actggaactt |
| 1561 | ccttttatga tgatgcccca ccctctaatt cctgtcagcc tacctccagc atctgtcacc |
| 1621 | atggcaatga gccagatgaa ccacctcagc accattgcaa atatggcagc agcagcacaa |
| 1681 | gttcagagtc ccccatccag agttgagaca tcagttatta aggagcgtgt tcctgatagc |
| 1741 | ccctcacctg ccccctctct ggaggagggg agaaggcctg gcagtcaccc atcatcacat |
| 1801 | cgcagcagca gcgtgtccag ctcccctgct cggactgaga gctcttctga cagaatcccg |
| 1861 | gtccatcaga atgggttgtc catgaaccag atgctgatgg gcttatcacc aaatgtactt |
| 1921 | cctgggccca aagagggaga tttggccggt catgacatgg gacatgagtc aaaaaggatg |
| 1981 | catattgaaa aagatgagac cccgctttct acaccaaccg caagagacag ccttgacaaa |
| 2041 | ctctctctaa ctgggcatgg acaaccactg cctccaggtt ttccatctcc ttttctgttt |
| 2101 | cctgatggac tgtcttccat cgagactctt ctgactaaca tacaggggct gttgaaagtt |
| 2161 | gccatagata atgccagagc tcaagagaaa caggtccaac tggaaaaaac tgagctgaag |
| 2221 | atggattttt taagggaaag agaactaagg gaaacacttg agaagcagtt ggctatggaa |
| 2281 | caaaagaata gagccatagt tcaaaagagg ctaaagaagg agaagaaggc aaagagaaaa |
| 2341 | ttgcaggaag cacttgagtt tgagacgaaa cggcgtgaac aagcagaaca gacgctaaaa |
| 2401 | caggcagctt caacagatag tctcagggtc ttaaatgact ctctgacccc agagatagag |
| 2461 | gctgaccgca gtggcggcag aacagatgct gaaaggacaa tacaagatgg aagactgtat |
| 2521 | ttgaaaacta ctgtcatgta ctgaatcttt cctgttgaag aaatccatgt tatagaaaag |
| 2581 | aactttgcag tcagacattc gtcatgggaa agttcagaaa aaaataaagt ccttttaagg |
| 2641 | gaacttcctg aattttgtgt attaatgttc tttaaaagtt taagtattct acaaaaaaaa |
| 2701 | aaaaagtttt ctccattgat tttcacctgt ggttcatacc agagacctga gaatgtttgt |
| 2761 | aaatgtacaa gtatcaaagt tcttacagtt aattactgca acttgctgct ggacaattgt |
| 2821 | atacagagtt aaaggcaggt ctgaataaga cctagctttg tttttttcta atggaatgaa |
| 2881 | ccattttcct cttctgaaaa ttctgtatct gagcacatca agagactctt gtagcagtgg |
| 2941 | ttacccagac ttacagaatt atgtcctcca gaaaccagca agaacacttg gaatgaacga |
| 3001 | atgaacttgt agggggcata gaggattctt gaaaaaaaaa aatgcaagag tgattttctg |
| 3061 | ttacattcaa tttcaaactc tctaattgtg ggttttctcc tgaagaattt tttttcacat |
| 3121 | actttccaaa agaccaacaa atggatgttg acaacaaccc aatgaaataa cattttgcat |
| 3181 | atctgaaaag aagcattgaa tataagccaa aagctttcac tgaaggtttt tttttcttaa |
| 3241 | aaataaaaaa aaatatataa gtgtaacatg ttttcattcc aaactggtag tggtatatag |
| 3301 | aattaaagat aataatgttg cttcttattc aaactgttgg tcatatgtac agtatataaa |
| 3361 | cataaaacac acaaggaagg tattatgtat gcagtagtat actagagttt aggaaaatga |
| 3421 | aaattttaga aaatatgttt tgtcaccctg ttggtcagaa agatgtcttt ctggttttaa |
| 3481 | cgcatgcagg catgtaaata tttgtctgga gtcacagtat taatgaatga gatcttaagc |
| 3541 | atctggtgac atcagaactc tgtgtcagcc acttttattt gtatattgaa ccctagctag |
| 3601 | tgccccaagc tgcactattg ggaatggatt gtggctgaac agcaaatcaa aacaccagaa |
| 3661 | atatttttat atgttaacgt catattatgt taatgttgct gaaaacaaaa cctaacaaac |
| 3721 | cttgatgtac cagtccaata ccatgtagcg ctgagtgata aagttaaaat gtgctgtgct |
| 3781 | tcccaccctt gtcagaggga agggtggcta tgtgttattt tcactgtctt tttgaaagtt |
| 3841 | acagtatgtg ttttcacttt cgtgcagata actggaagta aagcggcaaa cagtgcttat |
| 3901 | tacatgctaa agttaccttc tctttgtttt ttgcatatct ggaattacac ctttaaagac |
| 3961 | tgatatgaat cagtacggtc actatacatt ttatgatttt tctgtcatct taaaattgta |
| 4021 | tgatcgtaac attatttatt accacaaaac agcaaaatct tcaatgtcta agaaaactag |
| 4081 | cttaaaatgt ttaaatatag ttctgattgg gtattaatta cttgattaag aaaaaattaa |
| 4141 | cattatagat actctggcat tacgcttcta taccttttag gtcttccttg caatactgga |
| 4201 | acataattct tttgtgtagc tcactattag ccagctaagt tcatcttttt aataccataa |
| 4261 | aaaggttata tgtacagttc ctattttagc ttgcttacaa agggagcatt atttttattt |
| 4321 | aaagtattgc tagtaaatga tttgtagaaa cttggttttc taagcatagt tcttccataa |
| 4381 | ccaccttttg ttgtttgagc acaagggatt cttttcctag ttctatgtgt ttgtttccct |
| 4441 | atatgcagtc tttaaaggat tacaacactt aaaattgaat ggacttgtgt caagcttttt |
| 4501 | gcatcataca ttttttgaaa gatttttaaa aaagcctaca acttacatat gtagtagaat |
| 4561 | cagccattgc tctgctcctg gcatagagtc acctgtttgt tatgtggatt aaatagtttt |
| 4621 | aaaatacata tttgaagacc tttgagaatg ctttagtgtt tgatttgaaa taaaaggaaa |
| 4681 | ttttagcaag gattaaagaa aaaagctatc agctgtatgt taagagagac tcttactaac |
| 4741 | atgttgtaaa tattacaatt catgaaatgt tattgtaagt ctgtaactta attttttccc |
| 4801 | tgttttagtt atacaggttg gtttggaaat ttgtgttttg gcataaacaa gtaaaatgtg |
| 4861 | cccattttat ggtttccatg cttttgtaat cctaaaaata ttaatgtcta gttgttctat |
| 4921 | attataacca catttgcgct ctatgcaagc ccttggaaca gaacatactc atcttcatgt |
| 4981 | aggacctatg aaaattgtct atttttatct atatatttaa agttttctaa aaatgataaa |
| 5041 | aggttattac gaattttgtt gtacaaaatc tgtacaaaaa tctgttttta catcataatg |
| 5101 | caagaattgg aaatttttct atggtagcct agttatttga gcctggtttc aatgtgagaa |
| 5161 | ccacgtttac tgttattgta tttaattttc ttttcctttt caacaatctc ctaataaaac |
| 5221 | tgtctgaaat ctcaaaaaaa |

IL1R1 (SEQ ID NO: 15) - Homo sapiens interleukin 1 receptor type
1 (IL1R1), transcript variant 1, mRNA - NM_000877

| | |
|---|---|
| 1 | gtggccggcg gccggagccg actcggagcg cgcggcgccg gccgggagga gccggagagc |
| 61 | ggccgggccg ggcggtgggg gcgccggcct gccccgcgcg ccccagggag cggcaggaat |
| 121 | gtgacaatcg cgcgcccgcg caccgaagca ctcctcgctc ggctcctagg gctctcgccc |
| 181 | ctctgagctg agccgggttc cgcccggggc tgggatccca tcaccctcca cggccgtccg |
| 241 | tccaggtaga cgcaccctct gaagatggtg actccctcct gagaagctgg accccttggt |
| 301 | aaaagacaag gccttctcca agaagaatat gaaagtgtta ctcagactta tttgtttcat |
| 361 | agctctactg atttcttctc tggaggctga taaatgcaag gaacgtgaag aaaaaataat |
| 421 | tttagtgtca tctgcaaatg aaattgatgt tcgtccctgt cctcttaacc caaatgaaca |
| 481 | caaaggcact ataacttggt ataaagatga cagcaagaca cctgtatcta cagaacaagc |
| 541 | ctccaggatt catcaacaca aagagaaact ttggtttgtt cctgctaagg tggaggattc |
| 601 | aggacattac tattgcgtgg taagaaattc atcttactgc ctcagaatta aaataagtgc |
| 661 | aaaatttgtg gagaatgagc ctaacttatg ttataatgca caagccatat ttaagcagaa |
| 721 | actacccgtt gcaggagacg gaggacttgt gtgcccttat atggagtttt ttaaaaatga |
| 781 | aaataatgag ttacctaaat tacagtggta taaggattgc aaacctctac ttcttgacaa |
| 841 | tatacacttt agtggagtca aagataggct catcgtgatg aatgtggctg aaaagcatag |
| 901 | agggaactat acttgtcatg catcctacac atacttgggc aagcaatatc ctattacccg |
| 961 | ggtaatagaa tttattactc tagaggaaaa caaacccaca aggcctgtga ttgtgagccc |
| 1021 | agctaatgag acaatggaag tagacttggg atcccagata caattgatct gtaatgtcac |
| 1081 | cggccagttg agtgacattg cttactggaa gtggaatggg tcagtaattg atgaagatga |
| 1141 | cccagtgcta ggggaagact attacagtgt ggaaaatcct gcaaacaaaa gaaggagtac |
| 1201 | cctcatcaca gtgcttaata tatcggaaat tgaaagtaga ttttataaac atccatttac |
| 1261 | ctgttttgcc aagaatacac atggtataga tgcagcatat atccagttaa tatatccagt |
| 1321 | cactaatttc cagaagcaca tgattggtat atgtgtcacg ttgacagtca taattgtgtg |
| 1381 | ttctgttttc atctataaaa tcttcaagat tgacattgtg ctttggtaca gggattcctg |
| 1441 | ctatgatttt ctcccaataa aagcttcaga tggaaagacc tatgacgcat atatactgta |
| 1501 | tccaaagact gttggggaag ggtctacctc tgactgtgat atttttgtgt ttaaagtctt |
| 1561 | gcctgaggtc ttggaaaaac agtgtggata taagctgttc atttatggaa gggatgacta |
| 1621 | cgttggggaa gacattgttg aggtcattaa tgaaaacgta aagaaaagca gaagactgat |
| 1681 | tatcatttta gtcagagaaa catcaggctt cagctggctg ggtggttcat ctgaagagca |
| 1741 | aatagccatg tataatgctc ttgttcagga tggaattaaa gttgtcctgc ttgagctgga |
| 1801 | gaaaatccaa gactatgaga aaatgccaga atcgattaaa ttcattaagc agaaacatgg |
| 1861 | ggctatccgc tggtcagggg actttacaca gggaccacag tctgcaaaga caaggttctg |
| 1921 | gaagaatgtc aggtaccaca tgccagtcca gcgacggtca ccttcatcta aacaccagtt |
| 1981 | actgtcacca gccactaagg agaaactgca aagagaggct cacgtgcctc tcgggtagca |
| 2041 | tggagaagtt gccaagagtt ctttaggtgc ctcctgtctt atggcgttgc aggccaggtt |
| 2101 | atgcctcatg ctgacttgca gagttcatgg aatgtaacta tatcatcctt tatccctgag |
| 2161 | gtcacctgga atcagattat taagggaata agccatgacg tcaatagcag cccagggcac |
| 2221 | ttcagagtag agggcttggg aagatctttt aaaaaggcag taggcccggt gtggtggctc |
| 2281 | acgcctataa tcccagcact ttgggaggct gaagtgggtg gatcaccaga ggtcaggagt |
| 2341 | tcgagaccag cccagccaac atggcaaaac cccatctcta ctaaaaatac aaaaatgagc |
| 2401 | taggcatggt ggcacacgcc tgtaatccca gctacacctg aggctgaggc aggagaattg |
| 2461 | cttgaaccgg ggagacggag gttgcagtga gccgagtttg ggccactgca ctctagcctg |
| 2521 | gcaacagagc aagactccgt ctcaaaaaaa gggcaataaa tgccctctct gaatgtttga |
| 2581 | actgccaaga aaaggcatgg agacagcgaa ctagaagaaa gggcaagaag gaaatagcca |
| 2641 | ccgtctacag atggcttagt taagtcatcc acagcccaag ggcggggcta tgccttgtct |
| 2701 | ggggaccctg tagagtcact gaccctggag cggctctcct gagaggtgct gcaggcaaag |
| 2761 | tgagactgac acctcactga ggaagggaga catattcttg gagaactttc catctgcttg |
| 2821 | tattttccat acacatcccc agccagaagt tagtgtccga agaccgaatt ttattttaca |
| 2881 | gagcttgaaa actcacttca atgaacaaag ggattctcca ggattccaaa gttttgaagt |
| 2941 | catcttagct ttccacagga gggagagaac ttaaaaaagc aacagtagca gggaattgat |
| 3001 | ccacttctta atgctttcct ccctggcatg accatcctgt cctttgttat tatcctgcat |
| 3061 | tttacgtctt tggaggaaca gctccctagt ggcttcctcc gtctgcaatg tcccttgcac |
| 3121 | agcccacaca tgaaccatcc ttcccatgat gccgctcttc tgtcatcccg ctcctgctga |
| 3181 | aacacctccc aggggctcca cctgttcagg agctgaagcc catgctttcc caccagcatg |
| 3241 | tcactcccag accacctccc tgccctgtcc tccagcttcc cctcgctgtc ctgctgtgtg |
| 3301 | aattcccagg ttggcctggt ggccatgtcg cctgccccca gcactcctct gtctctgctc |
| 3361 | ttgcctgcac ccttcctcct cctttgccta ggaggccttc tcgcattttc tctagctgat |
| 3421 | cagaatttta ccaaaattca gaacatcctc caattccaca gtctctggga gactttccct |
| 3481 | aagaggcgac ttcctctcca gccttctctc tctggtcagg cccactgcag agatggtggt |
| 3541 | gagcacatct gggaggctgg tctccctcca gctggaattg ctgctctctg agggagaggc |
| 3601 | tgtggtggct gtctctgtcc ctcactgcct tccaggagca atttgcacat gtaacataga |
| 3661 | tttatgtaat gctttatgtt taaaaacatt ccccaattat cttatttaat ttttgcaatt |
| 3721 | attctaattt tatatataga gaaagtgacc tattttttaa aaaaatcaca ctctaagttc |
| 3781 | tattgaacct aggacttgag cctccatttc tggcttctag tctggtgttc tgagtacttg |
| 3841 | atttcaggtc aataacggtc ccccctcact ccacactggc acgtttgtga gaagaaatga |
| 3901 | cattttgcta ggaagtgacc gagtctagga atgcttttat tcaagacacc aaattccaaa |
| 3961 | cttctaaatg ttggaatttt caaaaattgt gtttagattt tatgaaaaac tcttctactt |
| 4021 | tcatctattc tttccctaga ggcaaacatt tcttaaaatg tttcattttc attaaaaatg |
| 4081 | aaagccaaat ttatatgcca ccgattgcag gacacaagca cagttttaag agttgtatga |
| 4141 | acatggagag gacttttggt ttttatattt ctcgtattta atatgggtga acaccaactt |
| 4201 | ttatttggaa taataatttt cctcctaaac aaaaacacat tgagtttaag tctctgactc |
| 4261 | ttgcctttcc acctgctttc tcctgggccc gctttgcctg cttgaaggaa cagtgctgtt |
| 4321 | ctggagctgc tgttccaaca gacagggcct agctttcatt tgacacacag actacagcca |
| 4381 | gaagcccatg gagcagggat gtcacgtctt gaaaagccta ttagatgttt tacaaattta |
| 4441 | attttgcaga ttattttagt ctgtcatcca gaaaatgtgt cagcatgcat agtgctaaga |
| 4501 | aagcaagcca atttggaaac ttaggttagt gacaaaattg gccagagagt gggggtgatg |
| 4561 | atgaccaaga attacaagta gaatggcagc tggaatttaa ggagggacaa gaatcaatgg |
| 4621 | ataagcgtgg gtggaggaag atccaaacag aaaagtgcaa agttattccc catcttccaa |
| 4681 | gggttgaatt ctggaggaag aagacacatt cctagttccc cgtgaacttc ctttgactta |
| 4741 | ttgtccccac taaaacaaaa caaaaaactt ttaatgcctt ccacattaat tagattttct |
| 4801 | tgcagttttt ttatggcatt tttttaaaga tgccctaagt gttgaagaag agtttgcaaa |
| 4861 | tgcaacaaaa tatttaatta ccggttgtta aaactggttt agcacaattt atattttccc |
| 4921 | tctcttgcct ttcttatttg caataaaagg tattgagcca ttttttaaat gacatttttg |
| 4981 | ataaattatg tttgtactag ttgatgaagg agtttttttt aacctgttta tataattttg |
| 5041 | cagcagaagc caaatttttt gtatattaaa gcaccaaatt catgtacagc atgcatcacg |
| 5101 | gatcaataga ctgtacttat tttccaataa aattttcaaa ctttgtactg ttaaaaaaaa |
| 5161 | aaaaaaaaaa |

OLAH (SEQ ID NO: 16) - Homo sapiens oleoyl-ACP hydrolase (OLAH),
transcript variant 2, mRNA - NM_001039702

| | |
|---|---|
| 1 | acctcatttc ctgtgtcctc tctttctttg gcaatccaaa gaaagtcatc tttcagattg |
| 61 | tctgctcaga gttcatctca aagcctggca aggattggag aggtcaataa gagtcagcgc |
| 121 | ctttaaaaag aaatctactc actcttctgt gtgcataagg ccgagcagag gttcttcgtc |
| 181 | tcaagaggaa ctgacttctg ttgagcactc aacacgccac agagaccagc catcttgcaa |
| 241 | cctcacctca cagcatggag agaggagacc aacctaagag aaccaggaat gaaaacattt |
| 301 | tcaactgctt atacaaaaac cctgaggcaa cttttaagct gatttgcttt ccctggatgg |
| 361 | gaggtggctc cactcatttt gccaaatggg gccaagatac tcatgatttg ctggaagtgc |
| 421 | actccttaag gcttcctgga agagaaagca gagttgaaga acctcttgaa aatgacatct |
| 481 | cccagttagt tgatgaagtt gtttgtgctc tgcagccagt catccaggat aaaccatttg |
| 541 | cattttttgg ccacagtatg ggatcctaca ttgcttttag gactgcacta ggtctaaaag |
| 601 | aaaacaatca accagaacca ttgcatttat ttttgtcaag tgcaactcct gtacattcaa |
| 661 | aggcctggca tcgcattccc aaagatgatg aattgtcaga agaacaaata agtcattacc |
| 721 | ttatggaatt tggaggcacc cccaagcatt ttgctgaagc caaggaattt gtgaaacaat |
| 781 | gtagtcccat cataagggca gatctgaaca ttgttagaag ttgcacctct aacgtaccat |
| 841 | ctaaggctgt tctttcctgt gacttgacat gttttgttgg atctgaagac atagcaaagg |
| 901 | acatggaagc ctggaaagat gtaaccagtg gaaatgctaa aatttaccag cttccagggg |
| 961 | gtcactttta tcttctggat cctgcgaacg agaaattaat caagaactac ataatcaagt |
| 1021 | gtctagaagt atcatcgata tccaattttt agatattttc cctttcactt ttaaaataat |
| 1081 | caaagtaata tcatactctt ctcagttatt cagatatagc tcagttttat tcagattgga |
| 1141 | aattacacat tttctactgt cagggagatt cgttacataa atatatttac gtatctgggg |
| 1201 | acaaaggtca agccagtaaa gaatacttct ggcagcactt tgggaggcca aggcgggcgg |
| 1261 | atcacgaggt caggagatcg agaccgtcct ggctaacacc gtgaaacccc atctctacta |
| 1321 | aaaatacaca aaattagccg ggcgtggtgg tgggcacctg tagtcccagc tactcgggag |
| 1381 | gctgaggcag gagaatggtg tgaacctggg aggtggagct tgcagtgaac cgagatcgct |
| 1441 | ccactgcact ccagcctggg tgacagatcc agactctgtc tcaaaaaaaa aaaaaaaaaa |
| 1501 | aatacttctg gcagagtctt ttatcttcct attaaaatct cacttgattc tcctttatgg |
| 1561 | gaagtttgtc gacaaaattc atgattagta aattatccat tttttccttc agttagttta |
| 1621 | atggtgaaga tgattaacag gggaaatgct tgaagtaaat gattgtttca atggc |

IL1R2 (SEQ ID NO: 17) - Homo sapiens interleukin 1 receptor type 2
(IRL1R2), transcript variant 1, mRNA- NM_004633

| | |
|---|---|
| 1 | cccgtgagga ggaaaaggtg tgtccgctgc cacccagtgt gagcgggtga caccacccgg |
| 61 | ttaggaaatc ccagctccca agagggtata aatccctgct ttactgctga gctcctgctg |
| 121 | gaggtgaaag tctggcctgg cagccttccc caggtgagca gcaacaaggc cacgtgctgc |
| 181 | tgggtctcag tcctccactt cccgtgtcct ctggaagttg tcaggagcaa tgttgcgctt |
| 241 | gtacgtgttg gtaatgggag tttctgcctt cacccttcag cctgcggcac acacaggggc |
| 301 | tgccagaagc tgccggtttc gtgggaggca ttacaagcgg gagttcaggc tggaagggga |
| 361 | gcctgtagcc ctgaggtgcc cccaggtgcc ctactggttg tgggcctctg tcagcccccg |
| 421 | catcaacctg acatggcata aaaatgactc tgctaggacg gtcccaggag aagaagagac |
| 481 | acggatgtgg gcccaggacg gtgctctgtg gcttctgcca gccttgcagg aggactctgg |
| 541 | cacctacgtc tgcactacta gaaatgcttc ttactgtgac aaaatgtcca ttgagctcag |
| 601 | agtttttgag aatacagatg ctttcctgcc gttcatctca tacccgcaaa ttttaacctt |
| 661 | gtcaacctct ggggtattag tatgccctga cctgagtgaa ttcacccgtg acaaaactga |
| 721 | cgtgaagatt caatggtaca aggattctct tcttttggat aaagacaatg agaaatttct |
| 781 | aagtgtgagg gggaccactc acttactcgt acacgatgtg gccctggaag atgctggcta |
| 841 | ttaccgctgt gtcctgacat ttgcccatga aggccagcaa tacaacatca ctaggagtat |
| 901 | tgagctacgc atcaagaaaa aaaaagaaga gaccattcct gtgatcattt cccccctcaa |
| 961 | gaccatatca gcttctctgg ggtcaagact gacaatcccg tgtaaggtgt ttctgggaac |
| 1021 | cggcacaccc ttaaccacca tgctgtggtg gacggccaat gacacccaca tagagagcgc |
| 1081 | ctacccggga ggccgcgtga ccgaggggcc acgccaggaa tattcagaaa ataatgagaa |
| 1141 | ctacattgaa gtgccattga tttttgatcc tgtcacaaga gaggatttgc acatggattt |
| 1201 | taaatgtgtt gtccataata ccctgagttt tcagacacta cgcaccacag tcaaggaagc |
| 1261 | ctcctccacg ttctcctggg gcattgtgct ggccccactt tcactggcct tcttggtttt |
| 1321 | ggggggaata tggatgcaca gacggtgcaa acacagaact ggaaaagcag atggtctgac |
| 1381 | tgtgctatgg cctcatcatc aagactttca atcctatccc aagtgaaata aatggaatga |
| 1441 | aataattcaa acacaaaaaa aaaaaaaaaa aaaaaaaaaa aaaa |

CYP19A1 (SEQ ID NO: 18) - Homo sapiens cytochrome P450 family 19
subfamily A member 1 (CYP19A1), transcript variant 2, mRNA -
NM_031226

| | |
|---|---|
| 1 | gggagtttct ggagggctga acacgtggag gcaaacagga aggtgaagaa gaacttatcc |
| 61 | tatcaggacg gaaggtcctg tgctcgggat cttccagacg tcgcgcggtg tcagaaaccc |
| 121 | tgtggtgaaa ttcagcctgt ggattccaga aatttggagt gttcttgggg ggaaaaatcc |
| 181 | gcacacacaa agcaacattt ggaaatccct gtggactcta aattgccccc tctgaggtca |
| 241 | aggaacacaa gatggttttg gaaatgctga acccgataca ttataacatc accagcatcg |
| 301 | tgcctgaagc catgcctgct gccaccatgc cagtcctgct cctcactggc ctttttctct |
| 361 | tggtgtggaa ttatgagggc acatcctcaa taccaggtcc tggctactgc atgggaattg |
| 421 | gacccctcat ctcccacggc agattcctgt ggatggggat cggcagtgcc tgcaactact |
| 481 | acaaccgggt atatggagaa ttcatgcgag tctggatctc tggagaggaa acactcatta |
| 541 | tcagcaagtc ctcaagtatg ttccacataa tgaagcacaa tcattacagc tctcgattcg |
| 601 | gcagcaaact tgggctgcag tgcatcggta tgcatgagaa aggcatcata tttaacaaca |
| 661 | atccagagct ctggaaaaca actcgaccct tctttatgaa agctctgtca ggccccggcc |
| 721 | ttgttcgtat ggtcacagtc tgtgctgaat ccctcaaaac acatctggac aggttggagg |
| 781 | aggtgaccaa tgaatcgggc tatgtggacg tgttgaccct tctgcgtcgt gtcatgctgg |
| 841 | acacctctaa cacgctcttc ttgaggatcc ctttggacga aagtgctatc gtggttaaaa |
| 901 | tccaaggtta ttttgatgca tggcaagctc tcctcatcaa accagacatc ttctttaaga |
| 961 | tttcttggct atacaaaaag tatgagaagt ctgtcaagga tttgaaagat gccatagaag |
| 1021 | ttctgatagc agaaaaaaga cgcaggattt ccacagaaga gaaactggaa gaatgtatgg |
| 1081 | actttgccac tgagttgatt ttagcagaga aacgtggtga cctgacaaga gagaatgtga |
| 1141 | accagtgcat attggaaatg ctgatcgcag ctcctgacac catgtctgtc tctttgttct |
| 1201 | tcatgctatt tctcattgca aagcacccta atgttgaaga ggcaataata aaggaaatcc |
| 1261 | agactgttat tggtgagaga gacataaaga ttgatgatat acaaaaatta aaagtgatgg |
| 1321 | aaaacttcat ttatgagagc atgcggtacc agcctgtcgt ggacttggtc atgcgcaaag |
| 1381 | ccttagaaga tgatgtaatc gatggctacc cagtgaaaaa ggggacaaac attatcctga |
| 1441 | atattggaag gatgcacaga ctcgagtttt tccccaaacc caatgaattt actcttgaaa |
| 1501 | attttgcaaa gaatgttcct tataggtact ttcagccatt tggctttggg ccccgtggct |
| 1561 | gtgcaggaaa gtacatcgcc atggtgatga tgaaagccat cctcgttaca cttctgagac |
| 1621 | gattccacgt gaagacattg caaggacagt gtgttgagag catacagaag atacacgact |
| 1681 | tgtccttgca cccagatgag actaaaaaca tgctggaaat gatctttacc ccaagaaact |
| 1741 | cagacaggtg tctggaacac tagagaaggc tggtcagtac ccactctgga gcatttctca |
| 1801 | tcagtagttc acatacaaat catccatcct tgccaatagt gtcatcctca cagtgaacac |
| 1861 | tcagtggccc atggcatttt ataggcatac ctcctatggg ttgtcaccaa gctaggtgct |
| 1921 | atttgtcatc tgctcctgtt cacaccagag aaccaggcta caagagaaaa agcagaggcc |
| 1981 | aagagtttga gggagaaata gtcggtgaag aaaccgtatc cataaagacc cgattccacc |
| 2041 | aaatgtgctt tgagaaggat aggccttcat taacaaaatg tatgtctggt tccccagtag |
| 2101 | agctctactg cctcaaccca aggggatttt tatgtctggg gcagaaacac tcaagttgat |
| 2161 | tagaaagacc aggccaatgt cagggtacct ggggccaaac ccacctgcta gtgtgaatta |
| 2221 | aagtacttta attttgtttt ctgtggaggt ggaaaagcaa cattcatagt ctttggagaa |
| 2281 | atgcttagaa attcagcatt tgacccttgc tgtgaattaa gcccaattaa ttcctgtttg |
| 2341 | tctacatatg atctgtctgt ggcaaaagtt taatcagagg aaattctttc ccagtctgtc |
| 2401 | gatttatgcc tcagccactt gcctgtgcta caattcattg tgttacctgt agattcaggt |
| 2461 | aatacaaact atatataatc atcaagtaat acaaactaat ttagtaatag cctgggttaa |
| 2521 | gtattattag ggccctgtgt ctgctgtaga aaaaaaaatt cacatgatgc acttcaaatt |
| 2581 | caaataaaaa tccttttggc atgttcccat ttttgcttag ctcaattagt gtggctaacc |
| 2641 | aagagataac tgtaaatgtg acattgattt gctcttacta cagcttcagt gattggggga |
| 2701 | ggaaaagtcc caacccaatg ggctcaaact tctaaggggt actcctctca tccccttatc |
| 2761 | cttctccctc gacattttct ccctctttct tcccatgacc ccaaagccaa gggcaacaga |
| 2821 | tcagtaaaga acgtggtcag agtagaaccc ctgaagtatt ttttaatcct acctcaaaat |
| 2881 | ttaacagtta cctgagagat ttaacattat ctagttcatt gaatcattgt atgtggtcat |
| 2941 | ggataaattg cacaccttgg aattcgcttt ctaaaggaaa tcaaatgaat ggaggaactt |
| 3001 | tccaaacacc actttacttg tgttatatag ccaatataac tatctctact gaatgtcatt |
| 3061 | gaaaaactaa aaaattaaac ttatttacaa ataggtaaat atttgtcatt gaatccattg |
| 3121 | ccatcccatt tgactgttct tttcatccta ctgtctagta ataagctgag tataagatga |
| 3181 | cagtgtaatc tccctgaaag caggagctac tttctttctt ttgtaatcta tttccatccc |
| 3241 | catttccctg tcctgtctcc ctgtattcac tcccaagctc agttctgaat agacattcct |
| 3301 | gctcagagat actcccaact gatgcagaaa ccaaataaag aggtaggtat tccaagaatt |
| 3361 | caagaatgga cattagtaaa gaataaaaca tttatttgag cttggaatta tttggatcat |
| 3421 | ctatatggcc taaaaatata tggactatgc ctgtgtacct gaatacgtat gtagtcaggt |
| 3481 | caagacaatc atccaaataa cttagacccc taaaagcaag gccaggattt gcaatttaat |
| 3541 | gtgtcccaat taattcactt gaaaattagt aacactctgt ttacgttgcc tctggctgga |
| 3601 | gctgcatggt ggaagaagcc caactttgga tccatgtact tcacccatcc aatactcttg |
| 3661 | ggacatttat gtgtatttta tctgtatata tgaagccaat gtctatgtct acacagtcaa |
| 3721 | agtgaaatgc atgtttgata tagctgtaca tagatatcta ttttgcaggt acaaaaatat |
| 3781 | cctgggggaa aactgggagt ggaagggtgg ggggtgggag tgagggacat gggggaggga |
| 3841 | caggaagagg agaagtgttg gtttgaacga tccaagcaaa ctctcccaga atcaaattac |
| 3901 | ctgggtagtt gttcaacttt tcactctgct tagcctgtat agacaaaccc catatatttg |
| 3961 | tagaggcttg gccttggaat tctggaatac cattggcttt tcagtaggct gatgaacaca |
| 4021 | ttttgaaaat tctattatct tcagaatttt gccccattgt taagtgctta accgtcactc |
| 4081 | ttgaatgtgc aatgtgctgt ggattccatt ttcatcagtt ctgaaagaac tgcaatgtgt |
| 4141 | aaattatcag tgaaatgcat gcatataagg gctctatcat tatcaaattg taaggacaat |
| 4201 | tgtacccttc tatatctttg ggcatgctag acacccccat gccttcattg agatcccatt |
| 4261 | ttccccctct caagtggaaa ataatcacat ccagcaagct ctctcattat tgagaaatac |
| 4321 | catttggaaa ttgccacttt ttattcctaa gcagcacctt tcactgttca tgatgctaat |
| 4381 | gttccacaaa agcatgtgcc attggcccac tgaaggatag agggaccctt ttcaatctat |
| 4441 | atcagctggg ctctgggact gaatctctca cctattcttg cagaaagaca tactaattaa |
| 4501 | accttgtcaa agtaaaaaaa aaaaaaaaaa a |

MMP8 (SEQ ID NO: 19) - Homo sapiens matrix metallopeptidase 8 (MMP8),
transcript variant 1, mRNA - NM_002424

| | |
|---|---|
| 1 | gacacatgat gctgtgaacg tcagggtgct cgccagggaa gggccctacc cagagggaca |
| 61 | gaaagaaagc caggaggggt agagtttgaa gagaagatca tgttctccct gaagacgctt |
| 121 | ccatttctgc tcttactcca tgtgcagatt tccaaggcct ttcctgtatc ttctaaagag |
| 181 | aaaaatacaa aaactgttca ggactacctg gaaaagttct accaattacc aagcaaccag |
| 241 | tatcagtcta caaggaagaa tggcactaat gtgatcgttg aaaagcttaa agaaatgcag |
| 301 | cgattttttg ggttgaatgt gacggggaag ccaaatgagg aaactctgga catgatgaaa |
| 361 | aagcctcgct gtggagtgcc tgacagtggt ggttttatgt taaccccagg aaaccccaag |
| 421 | tgggaacgca ctaacttgac ctacaggatt cgaaactata ccccacagct gtcagaggct |
| 481 | gaggtagaaa gagctatcaa ggatgccttt gaactctgga gtgttgcatc acctctcatc |
| 541 | ttcaccagga tctcacaggg agaggcagat atcaacattg ctttttacca aagagatcac |
| 601 | ggtgacaatt ctccatttga tggacccaat ggaatccttg ctcatgcctt tcagccaggc |
| 661 | caaggtattg gaggagatgc tcattttgat gccgaagaaa catggaccaa cacctccgca |
| 721 | aattacaact tgtttcttgt tgctgctcat gaatttggcc attctttggg gctcgctcac |
| 781 | tcctctgacc ctggtgcctt gatgtatccc aactatgctt tcagggaaac cagcaactac |
| 841 | tcactccctc aagatgacat cgatggcatt caggccatct atggactttc aagcaaccct |
| 901 | atccaaccta ctggaccaag cacacccaaa ccctgtgacc ccagtttgac atttgatgct |
| 961 | atcaccacac tccgtggaga aatacttttc tttaaagaca ggtacttctg gagaaggcat |
| 1021 | cctcagctac aaagagtcga aatgaatttt atttctctat tctggccatc ccttccaact |
| 1081 | ggtatacagg ctgcttatga agattttgac agagacctca ttttcctatt taaaggcaac |
| 1141 | caatactggg ctctgagtgg ctatgatatt ctgcaaggtt atcccaagga tatatcaaac |
| 1201 | tatggcttcc ccagcagcgt ccaagcaatt gacgcagctg ttttctacag aagtaaaaca |
| 1261 | tacttctttg taaatgacca attctggaga tatgataacc aaagacaatt catggagcca |
| 1321 | ggttatccca aaagcatatc aggtgccttt ccaggaatag agagtaaagt tgatgcagtt |
| 1381 | ttccagcaag aacatttctt ccatgtcttc agtggaccaa gatattacgc atttgatctt |
| 1441 | attgctcaga gagttaccag agttgcaaga ggcaataaat ggcttaactg tagatatggc |
| 1501 | tgaagcaaaa tcaaatgtgg ctgtatccac tttcagaatg ttgaagggaa gttcagcaag |
| 1561 | cattttcgtt acattgtgtc ctgcttatac ttttctcaat attaagtcat tgtttcccat |
| 1621 | cactgtatcc attctacctg tcctccgtga aaatatgttt ggaatattcc actatttgca |
| 1681 | gaggcttatt cagttcttac acattccatc ttacattagt gattccatca aagagaagga |
| 1741 | aagtaagcct ttttgtcacc tcaatattta ctatttcaat acttacatat ctgacttcta |
| 1801 | ggatttattg ttatattact tgcctatctg acttcataca tccctcagtt tcttaaaatg |
| 1861 | tcctatgtat atcttctaca tgcaatttag aactagattt tggttagaag taaggattat |
| 1921 | aaacaaccta gacagtaccc ttggccttta cagaaaatat ggtgctgttt tctacccttg |
| 1981 | gaaagaaatg tagatgatat gtttcgtggg ttgaattgtg tcccccataa aagatatgtt |
| 2041 | gaagttctaa ccccaggtac ccatgaatgt gagcttacca gggtctttgc agatgtaatt |
| 2101 | agttaagtta aggtgagatc acactgaatt agggtgggct ctaaatccat tatgactgtt |
| 2161 | gttcttataa gaagaagaga ggcatagtca cctaggggag gaggccgtat gaagacagag |
| 2221 | gcagagattg gagtgacgca tctccaagcc aaggaattcc aaggactgta agccaccagt |
| 2281 | agaagctttg aagaggcaag gaaggattcc ctccaatagc cttcaagtgt gaccctgctg |
| 2341 | acacctgcag aattcggact tctatcctcc aaaaccgtga gggaataaat ttcctttgtt |
| 2401 | ttaagccacc aactttgcaa tactttgtta cagcaaccct agacatgagg tactagacac |
| 2461 | agtacatcta cacatatgaa aatgaatcaa cacagaatgc agaagtagaa cccttgctaa |
| 2521 | ggactactgg gcatcttccc aggacagcag ccaaaagaga accaccactt cctctcctgc |
| 2581 | ctcctccttg ctctctccta gagtccaaac ccaaatgggc cagttggatc tgatgttcgt |
| 2641 | cagttcttta cttctatttc ctggggtact caggagggca cacactatag ataacttggg |
| 2701 | ttagctgcat aaaattcaat gtctcattaa gttgcattaa actgagctta gatgtgtaag |
| 2761 | tttgctaacg gatgggtttt tttgttaaga actataggat ttatgggacc aagtctagcg |
| 2821 | agtccagata tcaaaatcat tataatgtta tatttgctgt tattagaata taatatagct |
| 2881 | tattatacaa taaatatgta gactgtaaaa tatatttctc actagtacct cctattttct |
| 2941 | ttctctgttg aagtttttaa atcccacaga taattaaatt ggcaccttta tgcttgttca |
| 3001 | aaaattaaaa taatctatta aataagttca aattaaagat ttttacttca aatgac |

TGFA (SEQ ID NO: 20) - Homo sapiens transforming growth factor
alpha (TGFA), transcript variant 1, mRNA - NM_003236

| | |
|---|---|
| 1 | gtcagctgtg ccccggtcgc cgagtggcga ggaggtgacg gtagccgcct tcctatttcc |
| 61 | gcccggcggg cagcgctgcg gggcgagtgc cagcagagag gcgctcggtc ctccctccgc |
| 121 | cctcccgcgc cgggggcagg ccctgcctag tctgcgtctt tttcccccgc accgcggcgc |
| 181 | cgctccgcca ctcgggcacc gcaggtaggg caggaggctg gagagcctgc tgcccgcccg |
| 241 | cccgtaaaat ggtcccctcg gctggacagc tcgccctgtt cgctctgggt attgtgttgg |
| 301 | ctgcgtgcca ggccttggag aacagcacgt ccccgctgag tgcagacccg cccgtggctg |
| 361 | cagcagtggt gtcccatttt aatgactgcc cagattccca cactcagttc tgcttccatg |
| 421 | gaacctgcag gtttttggtg caggaggaca agccagcatg tgtctgccat tctgggtacg |
| 481 | ttggtgcacg ctgtgagcat gcggacctcc tggccgtggt ggctgccagc cagaagaagc |
| 541 | aggccatcac cgccttggtg gtggtctcca tcgtggccct ggctgtcctt atcatcacat |
| 601 | gtgtgctgat acactgctgc caggtccgaa aacactgtga gtggtgccgg gccctcatct |
| 661 | gccggcacga gaagcccagc gccctcctga agggaagaac cgcttgctgc cactcagaaa |
| 721 | cagtggtctg aagagcccag aggaggagtt tggccaggtg gactgtggca gatcaataaa |
| 781 | gaaaggcttc ttcaggacag cactgccaga gatgcctggg tgtgccacag accttcctac |
| 841 | ttggcctgta atcacctgtg cagccttttg tgggccttca aaactctgtc aagaactccg |
| 901 | tctgcttggg gttattcagt gtgacctaga gaagaaatca gcggaccacg atttcaagac |
| 961 | ttgttaaaaa agaactgcaa agagacggac tcctgttcac ctaggtgagg tgtgtgcagc |
| 1021 | agttggtgtc tgagtccaca tgtgtgcagt tgtcttctgc cagccatgga ttccaggcta |
| 1081 | tatatttctt tttaatgggc cacctcccca caacagaatt ctgcccaaca caggagattt |
| 1141 | ctatagttat tgttttctgt catttgccta ctggggaaga aagtgaagga ggggaaactg |
| 1201 | tttaatatca catgaagacc ctagctttaa gagaagctgt atcctctaac cacgagaccc |
| 1261 | tcaaccagcc caacatcttc catggacaca tgacattgaa gaccatccca agctatcgcc |
| 1321 | acccttggag atgatgtctt atttattaga tggataatgg ttttattttt aatctcttaa |
| 1381 | gtcaatgtaa aaagtataaa accccttcag acttctacat taatgatgta tgtgttgctg |
| 1441 | actgaaaagc tatactgatt agaaatgtct ggcctcttca agacagctaa ggcttgggaa |
| 1501 | aagtcttcca gggtgcggag atggaaccag aggctgggtt actggtagga ataaaggtag |
| 1561 | gggttcagaa atggtgccat tgaagccaca aagccggtaa atgcctcaat acgttctggg |
| 1621 | agaaaactta gcaaatccat cagcagggat ctgtcccctc tgttggggag agaggaagag |
| 1681 | tgtgtgtgtc tacacaggat aaacccaata catattgtac tgctcagtga ttaaatgggt |
| 1741 | tcacttcctc gtgagccctc ggtaagtatg tttagaaata gaacattagc cacgagccat |
| 1801 | aggcatttca ggccaaatcc atgaaagggg gaccagtcat ttattttcca ttttgttgct |
| 1861 | tggttggttt gttgctttat ttttaaaagg agaagtttaa ctttgctatt tattttcgag |
| 1921 | cactaggaaa actattccag taattttttt ttcctcattt ccattcagga tgccggcttt |
| 1981 | attaacaaaa actctaacaa gtcacctcca ctatgtgggt cttcctttcc cctcaagaga |
| 2041 | aggagcaatt gttcccctga gcatctgggt ccatctgacc catggggcct gcctgtgaga |
| 2101 | aacagtgggt cccttcaaat acatagtgga tagctcatcc ctaggaattt tcattaaaat |
| 2161 | ttggaaacag agtaatgaag aaataatata taaactcctt atgtgaggaa atgctactaa |
| 2221 | tatctgaaaa gtgaaagatt tctatgtatt aactcttaag tgcacctagc ttattacatc |
| 2281 | gtgaaaggta catttaaaat atgttaaatt ggcttgaaat tttcagagaa ttttgtcttc |
| 2341 | ccctaattct tcttccttgg tctggaagaa caatttctat gaattttctc tttatttttt |
| 2401 | tttataattc agacaattct atgacccgtg tcttcatttt tggcactctt atttaacaat |
| 2461 | gccacacctg aagcacttgg atctgttcag agctgacccc ctagcaacgt agttgacaca |
| 2521 | gctccaggtt tttaaattac taaaataagt tcaagtttac atcccttggg ccagatatgt |
| 2581 | gggttgaggc ttgactgtag catcctgctt agagaccaat caacggacac tggtttttag |
| 2641 | acctctatca atcagtagtt agcatccaag agactttgca gaggcgtagg aatgaggctg |
| 2701 | gacagatggc ggaagcagag gttccctgcg aagacttgag atttagtgtc tgtgaatgtt |
| 2761 | ctagttccta ggtccagcaa gtcacacctg ccagtgccct catccttatg cctgtaacac |
| 2821 | acatgcagtg agaggcctca catatacgcc tccctagaag tgccttccaa gtcagtcctt |
| 2881 | tggaaaccag caggtctgaa aaagaggctg catcaatgca agcctggttg gaccattgtc |
| 2941 | catgcctcag gatagaacag cctggcttat ttggggattt ttcttctaga aatcaaatga |
| 3001 | ctgataagca ttggatccct ctgccattta atggcaatgg tagtctttgg ttagctgcaa |
| 3061 | aaatactcca tttcaagtta aaaatgcatc ttctaatcca tctctgcaag ctccctgtgt |
| 3121 | ttccttgccc tttagaaaat gaattgttca ctacaattag agaatcattt aacatcctga |
| 3181 | cctggtaagc tgccacacac ctggcagtgg ggagcatcgc tgtttccaat ggctcaggag |
| 3241 | acaatgaaaa gcccccattt aaaaaaataa caaacatttt ttaaaaggcc tccaatactc |
| 3301 | ttatggagcc tggatttttc ccactgctct acaggctgtg acttttttta agcatcctga |
| 3361 | caggaaatgt tttcttctac atggaaagat agacagcagc caaccctgat ctggaagaca |
| 3421 | gggccccggc tggacacacg tggaaccaag ccagggatgg gctggccatt gtgtccccgc |
| 3481 | aggagagatg ggcagaatgg ccctagagtt cttttccctg agaaaggaga aaaagatggg |
| 3541 | attgccactc acccacccac actggtaagg gaggagaatt tgtgcttctg gagcttctca |
| 3601 | agggattgtg ttttgcaggt acagaaaact gcctgttatc ttcaagccag gttttcgagg |
| 3661 | gcacatgggt caccagttgc tttttcagtc aatttggccg ggatggacta atgaggctct |
| 3721 | aacactgctc aggagacccc tgccctctag ttggttctgg gctttgatct cttccaacct |
| 3781 | gcccagtcac agaaggagga atgactcaaa tgcccaaaac caagaacaca ttgcagaagt |
| 3841 | aagacaaaca tgtatatttt taaatgttct aacataagac ctgttctctc tagccattga |
| 3901 | tttaccaggc tttctgaaag atctagtggt tcacacagag agagagagag tactgaaaaa |
| 3961 | gcaactcctc ttcttagtct taataattta ctaaaatggt caacttttca ttatctttat |
| 4021 | tataataaac ctgatgcttt tttttagaac tccttactct gatgtctgta tatgttgcac |
| 4081 | tgaaaaggtt aatatttaat gttttaattt attttgtgtg gtaagttaat tttgatttct |
| 4141 | gtaatgtgtt aatgtgatta gcagttattt tccttaatat ctgaattata cttaaagagt |
| 4201 | agtgagcaat ataagacgca attgtgtttt tcagtaatgt gcattgttat tgagttgtac |
| 4261 | tgtaccttat ttggaaggat gaaggaatga atcttttttt cctaaatcaa aaaaaaaaaa |
| 4321 | aaaaaa |

VSTM1 (SEQ ID NO: 21) - Homo sapiens V-set and transmembrane domain
containing 1 (VSTM1), transcript variant 1, mRNA - NM_198481

| | |
|---|---|
| 1 | gtgagaagga aactgcaaga gtggggcaga gaaccagagt gtcagagcaa aacctcctct |
| 61 | atctgcacat cctggggacg aaccgggcag ccggagagct gcggccggcc cagtcccgct |
| 121 | ccgcctttga agggtaaaac ccaaggcggg gccttggttc tggcagaagg gacgctatga |
| 181 | ccgcagaatt cctctccctg ctttgcctcg ggctgtgtct gggctacgaa gatgagaaaa |
| 241 | agaatgagaa accgcccaag ccctccctcc acgcctggcc cagctcggtg gttgaagccg |
| 301 | agagcaatgt gaccctgaag tgtcaggctc attcccagaa tgtgacattt gtgctgcgca |
| 361 | aggtgaacga ctctgggtac aagcaggaac agagctcggc agaaaacgaa gctgaattcc |
| 421 | ccttcacgga cctgaagcct aaggatgctg ggaggtactt ttgtgcctac aagacaacag |
| 481 | cctcccatga gtggtcagaa agcagtgaac acttgcagct ggtggtcaca gataaacacg |
| 541 | atgaacttga agctccctca atgaaaacag acaccagaac catctttgtc gccatcttca |
| 601 | gctgcatctc catccttctc ctcttcctct cagtcttcat catctacaga tgcagccagc |
| 661 | acagttcatc atctgaggaa tccaccaaga gaaccagcca ttccaaactt ccggagcagg |
| 721 | aggctgccga ggcagattta tccaatatgg aaagggtatc tctctcgacg gcagaccccc |
| 781 | aaggagtgac ctatgctgag ctaagcacca gcgccctgtc tgaggcagct tcagacacca |
| 841 | cccaggagcc cccaggatct catgaatatg cggcactgaa agtgtagcaa gaagacagcc |
| 901 | ctggccacta aaggaggggg gatcgtgctg gccaaggtta tcggaaatct ggagatgcag |
| 961 | atactgtgtt tccttgctct tcgtccatat caataaaatt aagtttctcg tcttaaaaag |
| 1021 | aaaaaaaaaa aaaa |

FCER1A (SEQ ID NO: 22) Fc fragment of IgE receptor Ia, NM_002001.3

| | |
|---|---|
| 1 | tactaagagt ctccagcatc ctccacctgt ctaccaccga gcatgggcct atatttgaag |
| 61 | ccttagatct ctccagcaca gtaagcacca ggagtccatg aagaagatgg ctcctgccat |
| 121 | ggaatcccct actctactgt gtgtagcctt actgttcttc gctccagatg gcgtgttagc |
| 181 | agtccctcag aaacctaagg tctccttgaa ccctccatgg aatagaatat ttaaaggaga |
| 241 | gaatgtgact cttacatgta atgggaacaa tttctttgaa gtcagttcca ccaaatggtt |
| 301 | ccacaatggc agcctttcag aagagacaaa ttcaagtttg aatattgtga atgccaaatt |
| 361 | tgaagacagt ggagaataca aatgtcagca ccaacaagtt aatgagagtg aacctgtgta |
| 421 | cctggaagtc ttcagtgact ggctgctcct tcaggcctct gctgaggtgg tgatggaggg |
| 481 | ccagcccctc ttcctcaggt gccatggttg gaggaactgg gatgtgtaca aggtgatcta |
| 541 | ttataaggat ggtgaagctc tcaagtactg gtatgagaac cacaacatct ccattacaaa |
| 601 | tgccacagtt gaagacagtg gaacctacta ctgtacgggc aaagtgtggc agctggacta |
| 661 | tgagtctgag cccctcaaca ttactgtaat aaaagctccg cgtgagaagt actggctaca |
| 721 | attttttatc ccattgttgg tggtgattct gtttgctgtg gacacaggat tatttatctc |
| 781 | aactcagcag caggtcacat ttctcttgaa gattaagaga accaggaaag gcttcagact |
| 841 | tctgaaccca catcctaagc caaaccccaa aaacaactga tataattact caagaaatat |
| 901 | ttgcaacatt agtttttttc cagcatcagc aattgctact caattgtcaa acacagcttg |
| 961 | caatatacat agaaacgtct gtgctcaagg atttatagaa atgcttcatt aaactgagtg |
| 1021 | aaactggtta agtggcatgt aatagtaagt gctcaattaa cattggttga ataaatgaga |
| 1081 | gaatgaatag attcatttat tagcatttgt aaaagagatg ttcaatttca ataaaataaa |
| 1141 | tataaaacca tgtaacagaa tgcttctgag taaaaaaaaa aaaaaaaaaa aaaaaaaa |

KLRK1 (SEQ ID NO: 23) - Homo sapiens killer cell lectin like receptor
K1 (KLRK1), mRNA - NM_007360

| | |
|---|---|
| 1 | actaagtatc tccactttca attctagatc aggaactgag gacatatcta aattttctag |
| 61 | ttttatagaa ggcttttatc cacaagaatc aagatcttcc ctctctgagc aggaatcctt |
| 121 | tgtgcattga agactttaga ttcctctctg cggtagacgt gcacttataa gtatttgatg |
| 181 | gggtggattc gtggtcggag gtctcgacac agctgggaga tgagtgaatt tcataattat |
| 241 | aacttggatc tgaagaagag tgatttttca acacgatggc aaaagcaaag atgtccagta |
| 301 | gtcaaaagca aatgtagaga aaatgcatct ccattttttt tctgctgctt catcgctgta |
| 361 | gccatgggaa tccgtttcat tattatggta acaatatgga gtgctgtatt cctaaactca |
| 421 | ttattcaacc aagaagttca aattcccttg accgaaagtt actgtggccc atgtcctaaa |
| 481 | aactggatat gttacaaaaa taactgctac caattttttg atgagagtaa aaactggtat |
| 541 | gagagccagg cttcttgtat gtctcaaaat gccagccttc tgaaagtata cagcaaagag |
| 601 | gaccaggatt tacttaaact ggtgaagtca tatcattgga tgggactagt acacattcca |
| 661 | acaaatggat cttggcagtg ggaagatggc tccattctct cacccaacct actaacaata |
| 721 | attgaaatgc agaagggaga ctgtgcactc tatgcctcga gctttaaagg ctatatagaa |
| 781 | aactgttcaa ctccaaatac gtacatctgc atgcaaagga ctgtgtaaag atgatcaacc |
| 841 | atctcaataa aagccaggaa cagagaagag attacaccag cggtaacact gccaactgag |
| 901 | actaaaggaa acaaacaaaa acaggacaaa atgaccaaag actgtcagat ttcttagact |
| 961 | ccacaggacc aaaccataga acaatttcac tgcaaacatg catgattctc caagacaaaa |
| 1021 | gaagagagat cctaaaggca attcagatat ccccaaggct gcctctccca ccacaagccc |
| 1081 | agagtggatg ggctggggga ggggtgctgt tttaatttct aaaggtagga ccaacaccca |
| 1141 | ggggatcagt gaaggaagag aaggccagca gatcactgag agtgcaaccc caccctccac |
| 1201 | aggaaattgc ctcatgggca gggccacagc agagagacac agcatgggca gtgccttccc |
| 1261 | tgcctgtggg ggtcatgctg ccacttttaa tgggtcctcc acccaacggg gtcagggagg |
| 1321 | tggtgctgcc ccagtgggcc atgattatct taaaggcatt attctccagc cttaagtaag |
| 1381 | atcttaggac gtttcctttg ctatgatttg tacttgcttg agtcccatga ctgtttctct |
| 1441 | tcctctcttt cttccttttg gaatagtaat atccatccta tgtttgtccc actattgtat |
| 1501 | tttggaagca cataacttgt ttggtttcac aggttcacag ttaagaagga attttgcctc |
| 1561 | tgaataaata gaatcttgag tctcatgcaa aaaaaaaaaa aaaaaa |

KLRB1 (SEQ ID NO: 24) - Homo sapiens killer cell lectin like receptor
B1 (KLRB1), mRNA - NM_002258

| | |
|---|---|
| 1 | gcctcacaga attgagagtt tgttcttaca cacaagttta atgccacctt cctctgtctg |
| 61 | ccatggacca acaagcaata tatgctgagt taaacttacc cacagactca ggcccagaaa |
| 121 | gttcttcacc ttcatctctt cctcgggatg tctgtcaggg ttcaccttgg catcaatttg |
| 181 | ccctgaaact tagctgtgct gggattattc tccttgtctt ggttgttact gggttgagtg |
| 241 | tttcagtgac atccttaata cagaaatcat caatagaaaa atgcagtgtg gacattcaac |
| 301 | agagcaggaa taaaacaaca gagagaccgg gtctcttaaa ctgcccaata tattggcagc |
| 361 | aactccgaga gaaatgcttg ttattttctc acactgtcaa cccttggaat aacagtctag |
| 421 | ctgattgttc caccaaagaa tccagcctgc tgcttattcg agataaggat gaattgatac |
| 481 | acacacagaa cctgatacgt gacaaagcaa ttctgttttg gattggatta aatttttcat |
| 541 | tatcagaaaa gaactggaag tggataaacg gctctttttt aaattctaat gacttagaaa |
| 601 | ttagaggtga tgctaaagaa aacagctgta tttccatctc acagacatct gtgtattctg |
| 661 | agtactgtag tacagaaatc agatggatct gccaaaaaga actaacacct gtgagaaata |
| 721 | aagtgtatcc tgactcttga |

DAAM2 (SEQ ID NO: 25) - Homo sapiens dishevelled associated activator
of morphogenesis 2 (DAAM2), transcript variant 2, mRNA - NM_015345

| | |
|---|---|
| 1 | gggcggggga aggagcatct caggaaaggg gtccccggac tctggggctc tcagcacctg |
| 61 | cggtcgcaaa ccaacctcat gccctgactt taccaggcgt cgggactctg acttaaccgg |
| 121 | ggaatgaggg acttggtctg gcggcagatc acaatgagga cctagggcat ctgtctgctg |
| 181 | acgccccctg gcctgcagtg accatggccc cccgcaagag gagccaccat ggcctgggct |
| 241 | tcctgtgctg cttcgggggc agtgacatcc ccgaaatcaa cctccgggac aaccaccctc |
| 301 | tgcagttcat ggagttctcc agccccatcc cgaacgcaga ggagctcaac atccgctttg |
| 361 | cagagctggt ggatgaattg gatctcactg acaaaaaccg agaggctatg tttgcactgc |
| 421 | cccctgagaa gaaatggcag atctactgca gcaagaagaa ggagcaggag gaccccaaca |
| 481 | agctggcaac cagctggcct gactattaca tcgaccgcat caattccatg gctgcgatgc |
| 541 | agagtctgta cgcgtttgat gaggaggaga cggagatgag gaaccaagtc gtggaagacc |
| 601 | tgaagacagc cctccggaca cagcctatga ggtttgtgac ccgcttcatt gagctggagg |
| 661 | gcttgacctg tctgctaaat ttcctccgga gcatggacca cgccacctgt gagagccgca |
| 721 | tccacacctc actcattggc tgcatcaaag cattgatgaa caactcccag gggcgggcac |
| 781 | atgtgctggc acagcctgag gccattagta ccatagccca gagcctacgc acagagaaca |
| 841 | gcaagaccaa ggtggctgtg ctggagatcc tgggtgctgt gtgcctcgtg cctggtggcc |
| 901 | acaagaaggt gctgcaggcc atgctgcact accaggtgta tgcagcagag cgaacccgct |
| 961 | tccagaccct gctgaacgag ctagaccgaa gtctgggccg gtaccgggat gaagtgaatc |
| 1021 | tgaaaacagc catcatgtcc ttcatcaatg ctgtcctcaa tgctggagct ggagaggata |
| 1081 | atctggagtt ccgcctacat ctacggtatg aattcctgat gctgggtata cagcctgtga |
| 1141 | ttgacaagct ccggcaacat gaaaatgcca tcctggacaa acatttagac ttcttcgaga |
| 1201 | tggtgcggaa tgaggatgac ctggagctag ccaggaggtt tgacatggtc cacatcgaca |
| 1261 | ccaagagtgc ttcccagatg tttgagttga tccacaagaa gctgaagtac acggaggcct |
| 1321 | acccctgcct gctctctgtg ctgcaccact gcctgcagat gccctacaaa cggaacggtg |
| 1381 | gctacttcca gcagtggcag ctcctggacc gcatcctcca gcagattgtc ctccaggatg |
| 1441 | agcggggtgt ggaccctgac ctggctccct tggagaactt caatgtcaag aacatcgtca |
| 1501 | acatgctcat caacgagaat gaagtgaaac agtggcgaga ccaggcagag aagttccgga |
| 1561 | aagaacacat ggagcttgtg agccgtctgg agaggaagga gcgggaatgc gagacaaaga |
| 1621 | cattggagaa ggaagagatg atgcggacgc tgaacaaaat gaaggacaag ctggcccggg |
| 1681 | agtcccagga gctgcgccag gctcggggac aagtggcaga gctggtagcc cagctcagtg |
| 1741 | aactctcaac aggccctgta tcttccccac caccccctgg gggcccactc accttgtctt |
| 1801 | cctcaatgac aaccaatgac ctgcctccac cccctcctcc tctgcccttt gcctgttgtc |
| 1861 | cccctccccc accaccaccc cttcctcccg ggggaccccc gactccccca ggtgccccac |
| 1921 | cttgcctcgg catgggcctg cccctccctc aggaccccta ccccagcagt gacgtcccac |
| 1981 | tcaggaaaaa gcgtgtcccc cagccttctc acccactgaa gtccttcaac tgggtgaagc |
| 2041 | tgaatgagga gcgtgtccct ggcaccgtat ggaatgagat tgatgacatg caggtatttc |
| 2101 | ggatcctgga cctagaggat tttgaaaaga tgttttcagc ctaccagagg caccagaaag |
| 2161 | agctgggctc cactgaagac atctacctgg cttcccgcaa ggtcaaagag ctgtcggtca |
| 2221 | ttgatggccg gagggcccaa aactgcatca tccttctttc caagttgaag ctttctaacg |
| 2281 | aggagatccg gcaggccatc ttgaagatgg atgagcagga ggaccttgct aaggacatgc |
| 2341 | tggagcagct cctcaagttc atcccagaga agagtgacat tgacctcctg gaggagcaca |
| 2401 | agcatgaaat tgagcggatg gcccgtgctg accgcttcct ctatgaaatg agcaggattg |
| 2461 | accactacca gcagcgactg caagccctct tcttcaagaa gaaattccag gagcggctgg |
| 2521 | ctgaggcaaa gcccaaagtg gaagccatcc tgttggcctc ccgggagctg gtccgcagca |
| 2581 | agcgtcttag acagatgcta gaggtcatcc tagccatagg caacttcatg aacaaagggc |
| 2641 | agcgtggggg cgcctacggg ttccgggtgg ccagcctcaa caagatcgct gacaccaagt |
| 2701 | ccagcatcga cagaaacatc tctctgctcc attacctgat catgatcctg gagaagcatt |
| 2761 | ttcctgatat tctaaacatg ccttcagagc tgcaacatct tccagaagct gccaaagtca |
| 2821 | acctagcaga actggagaag gaggtgggca acctcaggag gggcctgaga gcggtggagg |
| 2881 | tgctggagta tcagaggcgc caggtacggg agcccagtga caagtttgtc cctgtcatga |
| 2941 | gcgacttcat cacggtgtcc agcttcagct tctccgagct ggaggaccag ctaaatgagg |
| 3001 | ccagggacaa gttcgccaag gccttgatgc acttcgggga gcatgacagc aagatgcagc |
| 3061 | cagacgaatt ctttggcatc tttgatacct tcttgcaggc cttctcagag gcccggcagg |
| 3121 | atctagaggc catgaggagg aggaaggagg aggaggagcg gcgggcgcgc atggaagcca |
| 3181 | tgctgaagga gcagagggaa cgtgagcggt ggcagcggca gcggaaggtc ctggctgcag |
| 3241 | gcagctcgct ggaggaggga ggagagttcg atgacctggt gtcggccctg cgctctgggg |
| 3301 | aggtcttcga caaggactta tgcaagctca agcgcagccg caagcgatca gggagccagg |
| 3361 | ccctggaagt tacccgggag cgggcaataa accggctaaa ttattgacct ggggaactag |
| 3421 | ccacacagga ggccgggaga cagggactgg tgagaatggg gctgagtgga ggaggtggtg |
| 3481 | atatttaaac catttggtgc ttggtttaga gccttgggct gggtcctggg atggggggct |
| 3541 | gtgtgtggct ggaccaggtg tctccccacg cttaccttaa ggggctcctc ttatctcccc |
| 3601 | ttcacatgat tccttctgtg ccctggcccc aggtattatt ctgaggctgc cttggatggc |
| 3661 | ctcaggccag gtaaccccag gctgaagggg ccctgctccc catcccctac catgggcacc |
| 3721 | catgtgctgg cacagaacag ttccagatct agactggaga ggtccacagc cttgtccaga |
| 3781 | gttcctgtgt agcacgggga gcaatgatgg agggagcccc tgagagggaa tctggtgagg |
| 3841 | gaatccagac tcccttctct caaggggagg ctcaacagaa cattgacctg ggggcaaact |
| 3901 | ttcctcttga atgggaacag aggaggcatt atatattcta gttagatcag ctctggtagg |
| 3961 | ttccagagaa cagtcaatgt tggaaggatg atgcagggac caaagccatc aggacagagt |
| 4021 | agcagtgtct gtttcccatg tcacaagtcc tctggcctct ccctgcatgt cttaagtatc |
| 4081 | tttcccttcc ttctctaccc tcacctccat cctgtctact aatccacagt cctagaagac |
| 4141 | tcaccttggg tttccacagc tatggctcac taccaggtgc ttgatgaatc tggcgagggg |
| 4201 | ctcaagacag acctcatgca tcaccacacc tcatgccttt tgggcatctc ccatgtcccc |
| 4261 | atctcctgga cacctggcca ttgttgtgaa gccagacagt gacctcaaat gttgccttgg |
| 4321 | agtcccctac agcccctcag cagagggcag cacttgaatg cttagctcca tcccatagtt |
| 4381 | ctctacttca tataaattgc tcaggccctc ccaccccttc tctaacacta gcttcaaggc |
| 4441 | agaagccaca gcagcctctg tccagcctgc aggtggccac ttggaaccat gtgtccactg |
| 4501 | gcgttgggga gttggttcct gagaggtctg agggccagag ctgccctcta cattaacatg |
| 4561 | ctgtctctaa gggtggcccc tcctctcagg cgttcagatg gtgcgaacag cagagcaggc |
| 4621 | aagggaaact ggggagatgg ggatggagga ggaaggctga tatcctctgg ggagcacatc |
| 4681 | acctgaaggt gccaaggagg aaggctgaga ggggggccac cccatttctg gtacccaatt |
| 4741 | tggttcttca gcccaacttg caaggggttc cttctggtcc tcccatccac tgccaccttc |
| 4801 | cattttgtcc atctcatgct ggccttggtg gatgggatgg ctgtatctag acaaaatttt |
| 4861 | tctaaaactc catcaaggct cttattcaat accacgttcc gagttggcct ttcatcttct |
| 4921 | ttgagactgg ccctgcctaa cctctaccat caatgagctc ttggcccttc tgcccttccc |
| 4981 | tgtgtttctc actttccaac ctaatccctg gctcagggtt attgccagtg gagactggtg |
| 5041 | agctgggcct actctcagct gcctatcttc tgcctttcac ttgcatccaa ctcctggggc |
| 5101 | tgggaccgta gtagctgcgg gggggaagaa acacagggtc ggtgagccca gcatgtgcgt |
| 5161 | tggtttgagg gggcgggcgg tgtgtgtgtg ttctggtggg agggatctga gcaagtgcaa |
| 5221 | gcctggctga cacaggtgtg aagaggccat cctggaaccc aggtgagggc aagatgaagg |
| 5281 | cttccaggca gaacagctgc agagagtttg gctatatgca tctgcagccc caagagctcc |
| 5341 | cactgcaaga caagtgttgg ggaagatggg aggttgtggg tgaggcctct aaaggtcctc |
| 5401 | tcccaaactg accaggctga tgtcaaccta accccctcag gggcagggaa caggggaggg |
| 5461 | ctccacaagc gtgtctggca ttcccaccca ccatggaaga ctggatacgc acctggaaac |
| 5521 | aaaaggacta tggaagctgt tcaagataca tttgatcttc agaaaagcag aatttggttc |
| 5581 | aactgttgac agaggacaca aatacgttgt tccagagctc agccttctca ctctaaaaga |
| 5641 | aagatatttt tctatttatt ttctacatct ggccagtggc tctggtgcta gatgccactg |
| 5701 | tagccagatc tccaacagtg ccttggacca tggactcata ctcaactgag taagaagggg |
| 5761 | ctggtgccca gtcggggtgg ctgagctggt ccttaatagg ttgtttcttg gtcttgcttt |
| 5821 | cttcatgccc tccccactgc tcctgccacc tttagataag tttctctagc taattttgtg |
| 5881 | gccaatgtaa aattcgtcat caacctaaca aacacaacct tctcagcagc atttctcccc |
| 5941 | tgtgatggaa ataaagtgtt tagggcagtg ggaggagaaa attctccagg tgaatgggga |
| 6001 | agggtctgtt ccagcctctc cctactccca tcccatttcc accaactggg gaactgtgac |
| 6061 | tatctatctc ccccgacttc taccagggat gccttcacgc caaggctgtt ctcaccagct |
| 6121 | gcctcagatg acaaatgagg ctaatggaca taatctacag tgtccttttt cacttgcacc |
| 6181 | ttttttataa gaatatattg taatactaaa aaatattaaa ttcataccat ccctacccag |
| 6241 | tctgcctaaa aa |

HLA-DRA (SEQ ID NO: 26) - Homo sapiens major histocompatibility
complex, class II, DR alpha (HLA-DRA), mRNA - NM_019111

| | |
|---|---|
| 1 | ttttaatggt cagactctat tacaccccac attctctttt cttttattct tgtctgttct |
| 61 | gcctcactcc cgagctctac tgactcccaa cagagcgccc aagaagaaaa tggccataag |
| 121 | tggagtccct gtgctaggat ttttcatcat agctgtgctg atgagcgctc aggaatcatg |
| 181 | ggctatcaaa gaagaacatg tgatcatcca ggccgagttc tatctgaatc ctgaccaatc |
| 241 | aggcgagttt atgtttgact ttgatggtga tgagattttc catgtggata tggcaaagaa |
| 301 | ggagacggtc tggcggcttg aagaatttgg acgatttgcc agctttgagg ctcaaggtgc |
| 361 | attggccaac atagctgtgg acaaagccaa cctggaaatc atgacaaagc gctccaacta |
| 421 | tactccgatc accaatgtac ctccagaggt aactgtgctc acaaacagcc ctgtggaact |
| 481 | gagagagccc aacgtcctca tctgtttcat agacaagttc accccaccag tggtcaatgt |
| 541 | cacgtggctt cgaaatggaa aacctgtcac cacaggagtg tcagagacag tcttcctgcc |
| 601 | cagggaagac caccttttcc gcaagttcca ctatctcccc ttcctgccct caactgagga |
| 661 | cgtttacgac tgcagggtgg agcactgggg cttggatgag cctcttctca agcactggga |
| 721 | gtttgatgct ccaagccctc tcccagagac tacagagaac gtggtgtgtg ccctgggcct |
| 781 | gactgtgggt ctggtgggca tcattattgg gaccatcttc atcatcaagg gattgcgcaa |
| 841 | aagcaatgca gcagaacgca gggggcctct gtaaggcaca tggaggtgat ggtgtttctt |
| 901 | agagagaaga tcactgaaga aacttctgct ttaatggctt tacaaagctg gcaatattac |
| 961 | aatccttgac ctcagtgaaa gcagtcatct tcagcatttt ccagccctat agccacccca |
| 1021 | agtgtggata tgcctcttcg attgctccgt actctaacat ctagctggct tccctgtcta |
| 1081 | ttgccttttc ctgtatctat tttcctctat ttcctatcat tttattatca ccatgcaatg |
| 1141 | cctctggaat aaaacataca ggagtctgtc tctgctatgg aatgccccat ggggcatctc |
| 1201 | ttgtgtactt attgtttaag gtttcctcaa actgtgattt ttctgaacac aataaactat |
| 1261 | tttgatgatc ttgggtggaa aaaaaaaaaa aaaaaaaaaa aaaaaaaaaa aa |

BCL11B (SEQ ID NO: 27) - Homo sapiens B-cell CLL/lymphoma 11B
(BCL11B), transcript variant 1, mRNA - NM_138576

| | |
|---|---|
| 1 | tgcgctttcc acctaccaga ccctgaaaga aagtgtcagg agccggtgca aaacccagtt |
| 61 | taagttcaag aagacatttg caagtgcaag aggccaagca gtttgaagaa gtgtaagaga |
| 121 | ttttttttcc ttcgaaagaa tatattttta aagaaaccag ccagtccgcg gaaagcaaca |
| 181 | gcagtttttt ttttttttgc ctctttttct tattttagat cgagaggttt ttcttgcttt |
| 241 | tcttcccttt tttttctttt tgcaaacaaa acaaaaaaca gcatagaaga aagagcaaaa |
| 301 | taaagaagaa gaagaggagg aagagaggga aagagaggaa gggaaaaaaa acaccaaccc |
| 361 | gggcagagga ggaggtgcgg cggcggcggc ggcggcggca gcggcggcag cggcgcggcg |
| 421 | gcggctcgga ccccctcccc cggctccccc catcagtgca gctctccggg cgatgccaga |
| 481 | atagatgccg gggcaatgtc ccgccgcaaa cagggcaacc cgcagcactt gtcccagagg |
| 541 | gagctcatca ccccagaggc tgaccatgtg gaggccgcca tcctcgaaga agacgagggt |
| 601 | ctggagatag aggagccaag tggcctgggg ctgatggtgg gtggccccga ccctgacctg |
| 661 | ctcacctgtg gccagtgtca aatgaacttc cccttggggg acatcctggt ttttatagag |
| 721 | cacaaaagga agcagtgtgg cggcagcttg ggtgcctgct atgacaaggc cctggacaag |
| 781 | gacagcccgc caccctcctc acgctccgag ctcaggaaag tgtccgagcc ggtggagatc |
| 841 | gggatccaag tcacccccga cgaagatgac cacctgctct cacccacgaa aggcatctgt |
| 901 | cccaagcagg agaacattgc agggccgtgc aggcctgccc agctgccagc ggtggccccc |
| 961 | atagctgcct cctcccaccc tcactcatcc gtgatcactt cacctctgcg tgccctgggc |
| 1021 | gctctcccgc cctgcctccc cctgccgtgc tgcagcgcgc gcccggtctc gggtgacggg |
| 1081 | actcagggtg agggtcagac ggaggctccc tttggatgcc agtgtcagtt gtcaggtaaa |
| 1141 | gatgagcctt ccagctacat ttgcacaaca tgcaagcagc ccttcaacag cgcgtggttc |
| 1201 | ctgctgcagc acgcgcagaa cacgcacggc ttccgcatct acctggagcc cgggccggcc |
| 1261 | agcagctcgc tcacgccgcg gctcaccatc ccgccgccgc tcgggccgga ggccgtggcg |
| 1321 | cagtccccgc tcatgaattt cctgggcgac agcaacccct tcaacctgct gcgcatgacg |
| 1381 | ggccccatcc tgcgggacca cccgggcttc ggcgagggcc gcctgccggg cacgccgcct |
| 1441 | ctcttcagtc ccccgccgcg ccaccacctg gacccgcacc gcctcagtgc cgaggagatg |
| 1501 | gggctcgtcg cccagcaccc cagtgccttc gaccgagtca tgcgcctgaa ccccatggcc |
| 1561 | atcgactcgc ccgccatgga cttctcgcgg cggctccgcg agctggcggg caacagctcc |
| 1621 | acgccgccgc ccgtgtcccc gggccgcggc aaccctatgc accggctcct gaaccccttc |
| 1681 | cagcccagcc ccaagtcccc gttcctgagc acgccgccgc tgccgcccat gccccctggc |
| 1741 | ggcacgccgc ccccgcagcc gccagccaag agcaagtcgt gcgagttctg cggcaagacc |
| 1801 | ttcaagttcc agagcaatct catcgtgcac cggcgcagtc acacgggcga gaagccctac |
| 1861 | aagtgccagc tgtgcgacca cgcgtgctcg caggccagca agctcaagcg ccacatgaag |
| 1921 | acgcacatgc acaaggccgg ctcgctggcc ggccgctccg acgacgggct ctcggccgcc |
| 1981 | agctcccccg agcccggcac cagcgagctg gcgggcgagg gcctcaaggc ggccgacggt |
| 2041 | gacttccgcc accacgagag cgacccgtcg ctgggccacg agccggagga ggaggacgag |
| 2101 | gaggaggagg aggaggagga ggagctgcta ctggagaacg agagccggcc cgagtcgagc |
| 2161 | ttcagcatgg actcggagct gagccgcaac cgcgagaacg gcggtggtgg ggtgcccggg |
| 2221 | gtcccgggcg cggggggcgg cgcggccaag gcgctggctg acgagaaggc gctggtgctg |
| 2281 | ggcaaggtca tggagaacgt gggcctaggc gcactgccgc agtacggcga gctcctggcc |
| 2341 | gacaagcaga agcgcggcgc cttcctgaag cgtgcggcgg gcggcgggga cgcgggcgac |
| 2401 | gacgacgacg cgggcggctg cggggacgcg ggcgcgggcg gcgcggtcaa cgggcgcggg |
| 2461 | ggcggcttcg cgccaggcac cgagcccttc cccgggctct tcccgcgcaa gcccgcgccg |
| 2521 | ctgcccagcc ccgggctcaa cagcgccgcc aagcgcatca aggtggagaa ggacctggag |
| 2581 | ctgccgcccg ccgcgctcat cccgtccgag aacgtgtact cgcagtggct ggtgggctac |
| 2641 | gcggcgtcgc ggcacttcat gaaggacccc ttcctgggct tcacggacgc acgacagtcg |
| 2701 | cccttcgcca cgtcgtccga gcactcgtcc gagaacggca gcctgcgctt ctccacgccg |
| 2761 | cccggggacc tgctggacgg cggcctctcg ggccgcagcg gcacggccag cggaggcagc |
| 2821 | accccgcacc tgggcggccc gggccccggg cggcccagct ccaaggaggg ccgccgcagc |
| 2881 | gacacgtgcg agtactgcgg caaggtgttc aagaactgca gcaacttgac ggtgcaccgg |
| 2941 | cggagccaca ccggcgagcg gccttacaag tgcgagctgt gcaactacgc gtgcgcgcag |
| 3001 | agcagcaagc tcacgcgcca catgaagacg cacgggcaga tcggcaagga ggtgtaccgc |
| 3061 | tgcgacatct gccagatgcc cttcagcgtc tacagcaccc tggagaaaca catgaaaaag |
| 3121 | tggcacggcg agcacttgct gactaacgac gtcaaaatcg agcaggccga gaggagctaa |
| 3181 | gcgcgcgggc cccggcgccc cgcacctgta cagtggaacc gttgccaacc gagagaatgc |
| 3241 | tgacctgact tgcctccgtg tcaccgccac cccgcacccc gcgtgtcccc ggggcccagg |
| 3301 | ggaggcggca ctccaaccta acctgtgtct gcgaagtcct atggaaaccc gagggttgat |
| 3361 | taaggcagta caaattgtgg agccttttaa ctgtgcaata atttctgtat ttattgggtt |
| 3421 | ttgtaatttt tttggcatgt gcaggtactt tttattatta ttttttctgt ttgaattcct |
| 3481 | ttaagagatt ttgttgggta tccatccctt ctttgttttt tttttaaccc ggtagtagcc |
| 3541 | tgagcaatga ctcgcaagca atgttagagg ggaagcatat cttttaaatt ataatttggg |
| 3601 | gggaggggtg gtgctgcttt tttgaaattt aagctaagca tgtgtaattt cttgtgaaga |
| 3661 | agccaacact caaatgactt ttaaagttgt ttactttttc attccttcct tttttttgtc |
| 3721 | ctgaaataaa aagtggcatg cagttttttt tttaattatt ttttaatttt ttttttggtt |
| 3781 | tttgtttttg gggtgggggg tgtggatgta cagcggataa caatctttca agtcgtagca |
| 3841 | ctttgtttca gaactggaat ggagatgtag cactcatgtc gtcccgagtc aagcggcctt |
| 3901 | ttctgtgttg atttcggctt tcatattaca taagggaaac cttgagtggt ggtgctgggg |
| 3961 | gaggcacccc acagactcag cgccgccaga gatagggttt ttggagggct cctctgggaa |
| 4021 | atggcccgac agcattctga ggttgtgcat gaccagcaga tactatcctg ttggtgtgcc |
| 4081 | ctggggtgcc atggctgcta ttcgctgtag attaggctac ataaaatggg ctgagggtac |
| 4141 | ctttttgggg agatggggtg gcctgcagtg acacagaaag gaagaaacta gcggtgttct |
| 4201 | tttaggcgtt ttctggcttg acggcttctc tcttttttta aatcaccccc accacataaa |
| 4261 | tctcaaatcc tatgttgcta caaggggtca tccatcattt cccaagcaga cgaatgccct |
| 4321 | aattaattga agttagtgtt ctctcattta atgcacactg atgatattgt agggatgggt |
| 4381 | ggggtgggga tcttgcaaat ttctattctc ttttactgaa aaagcagggg atgagttcca |
| 4441 | tcagaaggtg cccagcgcta cttcccaggt ttttattttt tttttcctat ctcattaggt |
| 4501 | tggaaggtac taaatattga actgttaaga ttagacattt gaattctgtt gacccgcact |
| 4561 | ttaaagcttt tgtttgcatt taaattaaat ggcttctaaa caagaaattg cagcatattc |
| 4621 | ttctctttgg cccagaggtg ggttaaactg taagggacag ctgagattga gtgtcagtat |
| 4681 | tgctaagcgt ggcattcaca atactggcac tataaagaac aaaataaaat aataatttat |
| 4741 | aggacagttt ttctactgcc attcaatttg atgtgagtgc cttgaaaact gatcttccta |
| 4801 | tttgagtctc ttgagacaaa tgcaaaactt tttttttgaa atgaaaagac tttttaaaaa |
| 4861 | agtaaaacaa gaaaagtaca ttctttagaa actaacaaag ccacatttac tttaagtaaa |
| 4921 | aaaaaaaaaa attctggttg aagatagagg atatgaaatg ccataagacc caatcaaatg |
| 4981 | aagaaataaa cccagcacaa ccttggacat ccattagctg aattatcctc agcccctttt |
| 5041 | gtttttggga caacgctgct tagatatgga gtggaggtga tttactgctg aattaaaact |
| 5101 | caagtgacac aagttacaag ttgatatcgt tgaatgaaaa gcaaaacaaa aacaattcag |
| 5161 | gaacaacggc taattttttc taaagttaaa tttagtgcac tctgtcttaa aaatacgttt |
| 5221 | acagtattgg gtacatacaa gggtaaaaaa aaaattgtgt gtatgtgtgt tggagcgatc |
| 5281 | tttttttttc aaagtttgct taataggtta tacaaaaatg ccacagtggc cgcgtgtata |
| 5341 | ttgttttctt ttggtgacgg ggttttagta tatattatat atattaaaat ttcttgatta |
| 5401 | ctgtaaaagt ggaccagtat ttgtaataat cgagaatgcc tgggcatttt acaaaacaag |
| 5461 | aaaaaaaata cccttttctt ttccttgaaa atgttgcagt aaaatttaaa tggtgggtct |
| 5521 | ataaatttgt tcttgttaca gtaactgtaa agtcggagtt ttagtaaatt tttttctgcc |
| 5581 | ttgggtgttg aatttttatt tcaaaaaaaa tgtatagaaa cttgtatttg gggattcaaa |
| 5641 | ggggattgct acaccatgta gaaaaagtat gtagaaaaaa agtgcttaat attgttattg |
| 5701 | ctttgcagaa aaaaaaaaaa tcacatttct gacctgtact tatttttctc ttcccgcctc |
| 5761 | cctctggaat ggatatattg gttggttcat atgatgtagg cacttgctgt atttttactg |
| 5821 | gagctcgtaa ttttttaact gtaagcttgt ccttttaaag ggatttaatg tacctttttg |
| 5881 | ttagtgaatt tggaaataaa aagaaaaaaa aaacaaaaac aaacaggctg ccataatata |
| 5941 | tttttttaat ttggcaggat aaaatattgc aaaaaaaaca catttgtatg ttaagtccta |
| 6001 | ttgtacagga gaaaaagggt tgtttgacaa cctttgagaa aaagaaacaa aaggaagtag |
| 6061 | ttaaatgctt tggttcacaa atcatttagt tgtatatatt ttttgtcgga attggcctac |
| 6121 | acagagaacc gttcgtgttg ggcttctctc tgaacgcccc gaaccttgca tcaaggctcc |
| 6181 | ttggtgtggc cacagcagac cagatgggaa attatttgtg ttgagtggaa aaaaatcagt |
| 6241 | ttttgtaaag atgtcagtaa cattccacat cgtcctccct ttctctaaga ggccatctct |
| 6301 | aagatgtcag atgtagagga gagagagcga gagaacatct tccttctcta ccatcactcc |
| 6361 | tgtggcggtc accaccacca cctctcccgc ccttaccagc agaaagcaat gcaaactgag |
| 6421 | ctgctttagt ccttgagaaa ttgtgaaaca aacacaaata tcataaaagg agctggtgat |
| 6481 | tcagctgggt ccaggtgaag tgacctgctg ttgagaccgg tacaaattgg atttcaggaa |
| 6541 | ggagactcca tcacagccag gacctttcgt gccatggaga gtgttggcct cttgtctttc |
| 6601 | ttccctgctt tgctgctttg ctctctgaaa cctacattcc gtcagtttcc gaatgcgagg |
| 6661 | gcctgggatg aatttggtgc ctttccatat ctcgttctct ctccttcccc tgcgtttcct |
| 6721 | ctccatcctt catcctccat tggtcctttt tttttctttc attttttatt taatttcttt |
| 6781 | tcttcctgtc tgttcctccc ctaatcctct attttatttt tattttttgt aaagccaagt |
| 6841 | agctttaaga taaagtggtg gtcttttgga tgagggaata atgcattttt aaataaaata |
| 6901 | ccaatatcag gaagccattt tttatttcag gaaatgtaag aaaccattat ttcaggttat |
| 6961 | gaaagtataa ccaagcatcc ttttgggcaa ttccttacca aatgcagaag cttttctgtt |
| 7021 | cgatgcactc tttcctcctt gccacttacc tttgcaaagt taaaaaaaag gggggaggga |
| 7081 | atgggagaga aagctgagat ttcagtttcc tactgcagtt tcctacctgc agatccaggg |
| 7141 | gctgctgttg cctttggatg ccccactgag gtcctagagt gcctccaggg tggtcttcct |
| 7201 | gtagtcataa cagctagcca gtgctcacca gcttaccaga ttgccaggac taagccatcc |
| 7261 | caaagcacaa gcattgtgtg tctctgtgac tgcagagaag agagaatttt gcttctgttt |
| 7321 | tgtgtttaaa aaaccaacac ggaagcagat gatcccgaga gagaggcctc tagcatgggt |
| 7381 | gacccagccg acctcaggcc ggtttccgca ctgccacaac tttgttcaaa gttgccccca |
| 7441 | attggaacct gccacttggc attagagggt ctttcatggg gagagaagga gactgaatta |
| 7501 | ctctaagcaa aatgtgaaaa gtaaggaaat cagcctttca tcccggtcct aagtaaccgt |
| 7561 | cagccgaagg tctcgtggaa cacaggcaaa cccgtgattt tggtgctcct tgtaactcag |
| 7621 | ccctgcaaag caaagtccca ttgatttaag ttgtttgcat ttgtactggc aaggcaaaat |
| 7681 | atttttatta ccttttctat tacttattgt atgagctttt gttgtttact tggaggtttt |
| 7741 | gtcttttact acaagtttgg aactatttat tattgcttgg tatttgtgct ctgtttaaga |
| 7801 | aacaggcact tttttttatt atggataaaa tgttgagatg acaggaggtc atttcaatat |
| 7861 | ggcttagtaa aatatttatt gttcctttat tctctgtaca agattttggg cctctttttt |
| 7921 | tccttaatgt cacaatgttg agttcagcat gtgtctgcca tttcatttgt acgcttgttc |
| 7981 | aaaaccaagt ttgttctggt ttcaagttat aaaaataaat tggacattta acttgatctc |
| 8041 | caaa |

ITM2A (SEQ ID NO: 28) - Homo sapiens integral membrane protein 2A
(ITM2A), transcript variant 1, mRNA - NM_004867

| | |
|---|---|
| 1 | gtgcaggtga ggcacgttta gcctgagccg gccacggact ccacttcccc tgctcttccc |
| 61 | ccagtggcaa atccgcgcca cctcgcaaac ccccaactca ggcacttggg ccccttttgg |
| 121 | gccccctctc gctcctccct ttaggcacct ccctgggccc gcccacggtc tccccccagt |
| 181 | ttgggactgc gtcataagta tcccagacct cggcttgcag tagtgttaga ctgaagataa |
| 241 | agtaagtgct gtttgggcta acaggatctc ctcttgcagt ctgcagccca ggacgctgat |
| 301 | tccagcagcg ccttaccgcg cagcccgaag attcactatg gtgaaaatcg ccttcaatac |
| 361 | ccctaccgcc gtgcaaaagg aggaggcgcg gcaagacgtg gaggccctcc tgagccgcac |
| 421 | ggtcagaact cagatactga ccggcaagga gctccgagtt gccacccagg aaaaagaggg |
| 481 | ctcctctggg agatgtatgc ttactctctt aggcctttca ttcatcttgg caggacttat |
| 541 | tgttggtgga gcctgcattt acaagtactt catgcccaag agcaccattt accgtggaga |
| 601 | gatgtgcttt tttgattctg aggatcctgc aaattccctt cgtggaggag agcctaactt |
| 661 | cctgcctgtg actgaggagg ctgacattcg tgaggatgac aacattgcaa tcattgatgt |
| 721 | gcctgtcccc agtttctctg atagtgaccc tgcagcaatt attcatgact ttgaaaaggg |
| 781 | aatgactgct tacctggact tgttgctggg gaactgctat ctgatgcccc tcaatacttc |
| 841 | tattgttatg cctccaaaaa atctggtaga gctctttggc aaactggcga gtggcagata |
| 901 | tctgcctcaa acttatgtgg ttcgagaaga cctagttgct gtggaggaaa ttcgtgatgt |
| 961 | tagtaacctt ggcatcttta tttaccaact ttgcaataac agaaagtcct tccgccttcg |
| 1021 | tcgcagagac ctcttgctgg gtttcaacaa acgtgccatt gataaatgct ggaagattag |
| 1081 | acacttcccc aacgaattta ttgttgagac caagatctgt caagagtaag aggcaacaga |
| 1141 | tagagtgtcc ttggtaataa gaagtcagag atttacaata tgactttaac attaaggttt |
| 1201 | atgggatact caagatattt actcatgcat ttactctatt gcttatgctt taaaaaaagg |
| 1261 | aaaaaaaaaa actactaacc actgcaagct cttgtcaaat tttagtttaa ttggcattgc |
| 1321 | ttgttttttg aaactgaaat tacatgagtt tcattttttc tttgaattta tagggtttag |
| 1381 | atttctgaaa gcagcatgaa tatatcacct aacatcctga caataaattc catccgttgt |
| 1441 | tttttttgtt tgtttgtttt ttcttttcct ttaagtaagc tctttattca tcttatggtg |
| 1501 | gagcaatttt aaaatttgaa atattttaaa ttgtttttga actttttgtg taaaatatat |
| 1561 | cagatctcaa cattgttggt ttcttttgtt tttcattttg tacaactttc ttgaatttag |
| 1621 | aaattacatc tttgcagttc tgttaggtgc tctgtaatta acctgactta tatgtgaaca |
| 1681 | attttcatga gacagtcatt tttaactaat gcagtgattc tttctcacta ctatctgtat |
| 1741 | tgtggaatgc acaaaattgt gtaggtgctg aatgctgtaa ggagtttagg ttgtatgaat |
| 1801 | tctacaaccc tataataaat tttactctat acaaaaaaaa aaaaaaaaaa a |

SLAMF6 (SEQ ID NO: 29) - Homo sapiens SLAM family member 6 (SLAMF6),
transcript variant 2, mRNA - NM_052931

| | |
|---|---|
| 1 | agtttatgac agaagggcaa aaacattgac tgcctcaagg tctcaagcac cagtcttcac |
| 61 | cgcggaaagc atgttgtggc tgttccaatc gctcctgttt gtcttctgct ttggcccagg |
| 121 | gaatgtagtt tcacaaagca gcttaacccc attgatggtg aacgggattc tgggggagtc |
| 181 | agtaactctt cccctggagt ttcctgcagg agagaaggtc aacttcatca cttggctttt |
| 241 | caatgaaaca tctcttgcct tcatagtacc ccatgaaacc aaaagtccag aaatccacgt |
| 301 | gactaatccg aaacagggaa agcgactgaa cttcacccag tcctactccc tgcaactcag |
| 361 | caacctgaag atggaagaca caggctctta cagagcccag atatccacaa agacctctgc |
| 421 | aaagctgtcc agttacactc tgaggatatt aagacaactg aggaacatac aagttaccaa |
| 481 | tcacagtcag ctatttcaga atatgacctg tgagctccat ctgacttgct ctgtggagga |
| 541 | tgcagatgac aatgtctcat tcagatggga ggccttggga aacacacttt caagtcagcc |
| 601 | aaacctcact gtctcctggg accccaggat ttccagtgaa caggactaca cctgcatagc |
| 661 | agagaatgct gtcagtaatt tatccttctc tgtctctgcc cagaagcttt gcgaagatgt |
| 721 | taaaattcaa tatacagata ccaaaatgat tctgtttatg gtttctggga tatgcatagt |
| 781 | cttcggtttc atcatactgc tgttacttgt tttgaggaaa agaagagatt ccctatcttt |
| 841 | gtctactcag cgaacacagg gccccgagtc cgcaaggaac ctagagtatg tttcagtgtc |
| 901 | tccaacgaac aacactgtgt atgcttcagt cactcattca aacagggaaa cagaaatctg |
| 961 | gacacctaga gaaaatgata ctatcacaat ttactccaca attaatcatt ccaaagagag |
| 1021 | taaacccact ttttccaggg caactgccct tgacaatgtc gtgtaagttg ctgaaaggcc |
| 1081 | tcagaggaat tcgggaatga cacgtcttct gatcccatga gacagaacaa agaacaggaa |
| 1141 | gcttggttcc tgttgttcct ggcaacagaa tttgaatatc taggatagga tgatcacctc |
| 1201 | cagtccttcg gacttaaacc tgcctacctg agtcaaacac ctaaggataa catcatttcc |
| 1261 | agcatgtggt tcaaataata ttttccaatc cacttcaggc caaaacatgc taaagataac |
| 1321 | acaccagcac attgactctc tctttgataa ctaagcaaat ggaattatgg ttgacagaga |
| 1381 | gtttatgatc cagaagacaa ccacttctct ccttttagaa agcagcagga ttgacttatt |
| 1441 | gagaaataat gcagtgtgtt ggttacatgt gtagtctctg gagttggatg ggcccatcct |
| 1501 | gatacaagtt gagcatccct tgtctgaaat gcttgggatt agaaatgttt cagatttcaa |
| 1561 | ttttttttca gattttggaa tatttgcatt atatttagcg gttgagtatc caaatccaaa |
| 1621 | aatccaaaat tcaaaatgct ccaataagca tttcccttga gtttcattga tgtcgatgca |
| 1681 | gtgctcaaaa tctcagattt tggagcattt tggatattgg atttttggat ttgggatgct |
| 1741 | caacttgtac aatgtttatt agacacatct cctgggacat actgcctaac cttttggagc |
| 1801 | cttagtctcc cagactgaaa aaggaagagg atggtattac atcagctcca ttgtttgagc |
| 1861 | caagaatcta agtcatccct gactccagtg tctttgtcac caggcccttt ggactctacc |
| 1921 | tcagaaatat ttcttggacc ttccacttct cctccaactc cttgaccacc atcctgtatc |
| 1981 | caaccatcac cacctctaac ctgaatccta ccttaagatc agaacagttg tcctcacttt |
| 2041 | tgttcttgtc cctctccaac ccactctcca caagatggcc agagtaatgt ttttaatata |
| 2101 | aattggatcc ttcagtttcc tgcttaaaac cctgcaggtt tcccaatgca ctcagaaaga |
| 2161 | aatccagttt ccatggccct ggatggtctg gcccacctcc agcctcagct agcattaccc |
| 2221 | ttctgacact ctctatgtag cctccctgat cttctttcag ctcctctatt aaaggaaaag |
| 2281 | ttctttatgt taattattta catcttcctg caggcccttc ctctgcctgc tggggtcctc |
| 2341 | ctattcttta ggtttaattt taaatatgtc acctcctaag agaaaccttc ccagaccact |
| 2401 | ctttctaaaa tgaatcttct aggctgggca tggtggctca cacctgtaat ccctgtactt |
| 2461 | tgggaggcca aggggggaga tcacttgagg tcaggagttc aagaccagcc tggccaactt |
| 2521 | ggtgaaaccc cgtctttact aaaaatacaa aaaaattagc caggcgtggt ggtgcacccc |
| 2581 | taaaatccca gctacttgag agactgaggc aggagaatcg cttgaaccca ggaggtggag |
| 2641 | gttccagtga gccaaaatca tgccaatgta ttccagtctg ggtgacagag tgagactctg |
| 2701 | tctcaaaaaa taaataaata aaataaaatg aaatagatct tataaaaaaa a |

HLA-DPB1 (SEQ ID NO: 30) - Homo sapiens major histocompatibility
complex, class II, DP beta 1, mRNA - NM_002121

| | |
|---|---|
| 1 | gtcacagaag actacttggg ttcatggtct ctaatatttc aaacaggagc tccctttagc |
| 61 | gagtccttct tttcctgact gcagctcttt tcattttgcc atccttttcc agctccatga |
| 121 | tggttctgca ggtttctgcg gccccccgga cagtggctct gacggcgtta ctgatggtgc |
| 181 | tgctcacatc tgtggtccag ggcagggcca ctccagagaa ttaccttttc cagggacggc |
| 241 | aggaatgcta cgcgtttaat gggacacagc gcttcctgga gagatacatc tacaaccggg |
| 301 | aggagttcgc gcgcttcgac agcgacgtgg gggagttccg ggcggtgacg gagctggggc |
| 361 | ggcctgctgc ggagtactgg aacagccaga aggacatcct ggaggagaag cgggcagtgc |
| 421 | cggacaggat gtgcagacac aactacgagc tgggcgggcc catgaccctg cagcgccgag |
| 481 | tccagcctag ggtgaatgtt tccccctcca agaaggggcc cttgcagcac cacaacctgc |
| 541 | ttgtctgcca cgtgacggat ttctacccag gcagcattca agtccgatgg ttcctgaatg |
| 601 | gacaggagga aacagctggg gtcgtgtcca ccaacctgat ccgtaatgga gactggacct |
| 661 | tccagatcct ggtgatgctg gaaatgaccc cccagcaggg agatgtctac acctgccaag |
| 721 | tggagcacac cagcctggat agtcctgtca ccgtggagtg gaaggcacag tctgattctg |
| 781 | cccggagtaa gacattgacg ggagctgggg gcttcgtgct ggggctcatc atctgtggag |
| 841 | tgggcatctt catgcacagg aggagcaaga aagttcaacg aggatctgca taaacagggt |
| 901 | tcctgagctc actgaaaaga ctattgtgcc ttaggaaaag catttgctgt gtttcgttag |
| 961 | catctggctc caggacagac cttcaacttc caaattggat actgctgcca agaagttgct |
| 1021 | ctgaagtcag tttctatcat tctgctcttt gattcaaagc actgtttctc tcactgggcc |
| 1081 | tccaaccatg ttcccttctt cttagcacca caaataatca aaacccaaca tgactgtttg |
| 1141 | ttttccttta aaaatatgca ccaaatcatc tctcatcact tttctctgag ggttttagta |
| 1201 | gacagtagga gttaataaag aagttcattt tggtttaaac ataggaaaga agagaaccat |
| 1261 | gaaaatgggg atatgttaac tattgtataa tggggcctgt tacacatgac actcttctga |
| 1321 | attgactgta tttcagtgag ctgcccccaa atcaagttta gtgccctcat ccatttatgt |
| 1381 | ctcagaccac tattcttaac tattcaatgg tgagcagact gcaaatctgc ctgataggac |
| 1441 | ccatattccc acagcactaa ttcaacatat accttactga gagcatgttt tatcattacc |
| 1501 | attaagaagt taaatgaaca tcagaattta aaatcataaa tataatctaa tacactttaa |
| 1561 | ccattttctt tgtgtgccat cacaaatact ccttaaccaa atacggcttg gacttttgaa |
| 1621 | tgcatccaat agacgtcatt tgtcgtctaa gtctgcattc atccaccagc ctaggcctcc |
| 1681 | tgtcttaatt ttcatacaga cagaaatgac tccccactgg ggaaagagca aagcaataca |
| 1741 | tgtagcactc tttttcaaac actggtcttt ttttttttct taacaatcca acattgttat |
| 1801 | gtgttttgcg tctcatattg acaccttttg gtcaaggtag aggacatgtt tgttgtaagc |
| 1861 | tttctttttc gtgtagagga tggattcttc actcctgata cacacaatca gtgcacagca |
| 1921 | gctctcttat acatccagtt gatgccttca gtctccctgg cttcttacaa gcatcttctg |
| 1981 | ggccttgtgt gtccctgggc acctgtccct ggtcaattcc cgaaagctac tgtgctcctc |
| 2041 | ttgcccatct ccccttgcaa ataatatctt ccatcggggg accggcttcc tccaatttca |
| 2101 | ggagaggtgg ggctgaaggc acagacttgg gcgtcactgg cacagatata agtaaataca |
| 2161 | gctggagtct gcagagaggc tggactgagt cagggagtca ggaaagagaa gccacacaca |
| 2221 | aggacaacca atcatgtttc tcataatctt cttaacctag ggaataggac acaatcattt |
| 2281 | tttcttttta aaacatcttt atccctgatc agcctcattt cctcaaaaac tataaaggaa |
| 2341 | aatgctgctg acttgttttt gcgtagtaat ttcagctgtc acataataag ctaaggaaga |
| 2401 | cagtatatag taaataagga ccctttatct gtcttatttt cccttttggc ttcacaggaa |
| 2461 | acttgtgaga aacctatgca gcataaaatt aatatgattt caatccaggg attcaacgat |
| 2521 | ggaaggaggt catgagaata gcagaaagtc ttcaaatcga gatcattatg aaatcctcag |
| 2581 | acccagagca cataaatcct accctcagag tcactgagca gttaacatta caaattacaa |
| 2641 | accatatcca gtcagagtca ttctctttcc tgcttgtctc ctgtactcat gttacaggtt |
| 2701 | agggcagtac cccgagtgga gtgaacaatc tctggactaa cacttgtcag gatcagaagc |
| 2761 | tgaggtatct gcacccacat tacaggaaca ggatatgtgc tcctagggaa ctgagggtgt |
| 2821 | caggagatga ggaatgtccc tggagtcaca gaaagaaggt atcagatgtg tctcactctg |
| 2881 | acatatgcag gtgtttatga aactctggga tttctaagga aggatgcagt gcagagacag |
| 2941 | gtcccagagg agacaagagc tgagagacca tccaaactgg gaccaccttg tcactagact |
| 3001 | tcaaattttc aatattgata gagtgttttc taagagtcag gccctttgct gagtgctatg |
| 3061 | tgcagcagga tcaaaggcag ccaggaggta gaggagtctt gaggtacatc agtcattgga |
| 3121 | gttgaagagc agagattcaa aggaaagttg gaactggagc tttaaaggag atgtgaagtg |
| 3181 | ggtgactcaa cctctgactc agaaaaattg atacctgcag aagaaaaaac ccggcgggct |
| 3241 | taggactccc agctgagtgt tgtatcctcc atccctttcc acctggtccc ttcattttct |
| 3301 | acccctcaca gttccctaac gagaaggtgg tccacccaac agacaacact gcctcagatg |
| 3361 | gttatcaagg ggtaccctaa gaagaaatca tctcaccctc tctttgtccc catttgtcaa |
| 3421 | gtagcagtga ggccgagcca ggggatggtg aaagtggaag gaggtgggag ttgggcatcg |
| 3481 | ggtgtgaaga tgctcttgaa aggggtttta ataaccactt gctaccaggc cagtgaacac |
| 3541 | ttaccatagt tgatgccttt tgagcatgtt gcattgtaaa ctgtccctga aattactgtg |
| 3601 | cacttggctt atgggatgaa acatcctcct agttcttttg tctctcagct tctctgaagt |
| 3661 | ctcattgagc accttctctt caatttcttt tacacagtaa gaataggatc agctgtgcta |
| 3721 | aactaacaaa tacccagata tccaggtttg gctcatgtta cacgtccaaa gtaagtcatg |
| 3781 | caggaagctc tgctcatcat cgtactcagg aagtcaggct gacagtcttt ctcctgcaca |
| 3841 | tctgctccca gaacctcccc agcagaatga agggaaccta agaatttatt cactggcttt |
| 3901 | taatgatccc tcctagaaag aacacacttc tcgcatttca ttttccaatg taaatcatat |
| 3961 | ggctgcaact aacttcaaat aagtgggaat acttgaaggt ggaaaacatt taagaagtac |
| 4021 | acactaaata aataataaaa tacttctaca agaga |

HLA-DPB1 (SEQ ID NO: 31) - Homo sapiens major histocompatibility
complex, class II, DP beta 1 (HLA-DPB1), transcript variant X1,
mRNA - XM_006725998

| | |
|---|---|
| 1 | atcactcagt gcccctgagc tcattctttt cagtaaattc tctctctgcg tggtgagaaa |
| 61 | acaggcctgg agaggctctg cgacccgctt aggaccacag aactcgagaa ttaccttttc |
| 121 | cagggacggc aggaatgcta cgcgtttaat gggacacagc gcttcctgga gagatacatc |
| 181 | tacaaccggg aggagctcgt gcgcttcgac agcgacgtgg gggagttccg ggcggtgacg |
| 241 | gagctggggc ggcctgaggc ggagtactgg aacagccaga aggacatcct ggaggaggag |
| 301 | cgggcagtgc cggacaggat gtgcagacac aactacgagc tgggcgggcc catgaccctg |
| 361 | cagcgccgag tccagcctag ggtgaatgtt tccccctcca agaaggggcc cttgcagcac |
| 421 | cacaacctgc ttgtctgcca cgtgacggat ttctacccag gcagcattca agtccgatgg |
| 481 | ttcctgaatg gacaggagga aacagctggg gtcgtgtcca ccaacctgat ccgtaatgga |
| 541 | gactggacct tccagatcct ggtgatgctg gaaatgaccc cccagcaggg agatgtctac |
| 601 | acctgccaag tggagcacac cagcctggat agtcctgtca ccgtggagtg gaaggcacag |
| 661 | tctgattctg cccggagtaa gacattgacg ggagctgggg gcttcgtgct ggggctcatc |
| 721 | atctgtggag tgggcatctt catgcacagg aggagcaaga aagttcaacg aggatctgca |
| 781 | taaacagggt tcctgagctc actgaaaaga ctattgtgcc ttaggaaaag catttgctgt |
| 841 | gtttcgttag catctggctc caggacagac cttcaacttc caaattggat actgctgcca |
| 901 | agaagttgct ctgaagtcag tttctatcat tctgctcttt gattcaaagc actgtttctc |
| 961 | tcactgggcc tccaaccatg ttcccttctt cttagcacca caaataatca aaacccaaca |
| 1021 | tgactgtttg ttttccttta aaaatatgca ccaaatcatc tctca |

CD160 (SEQ ID NO: 32) - CD160 molecule, transcript variant 1, mRNA -
NM_007053

| | |
|---|---|
| 1 | gacgaaactg gagagatagg gttttaacaa gatgcaagga caatctgagg actgagagcc |
| 61 | atttcaacgt gagcccccag tctgagaaca agaaagaaga acttctgtct cgagggtctc |
| 121 | actgtcaacc aggccagagt gcagtgaaga tcatacctca ctacatccgt gaactcccgg |
| 181 | gctcctccca cctaagtctc ttgagtagct gggacttcag gagactgaag ccaaggatac |
| 241 | cagcagagcc aacatttgct tcaagttcct gggcctgctg acagcgtgca ggatgctgtt |
| 301 | ggaacccggc agaggctgct gtgccctggc catcctgctg gcaattgtgg acatccagtc |
| 361 | tggtggatgc attaacatca ccagctcagc ttcccaggaa ggaacgcgac taaacttaat |
| 421 | ctgtactgta tggcataaga aagaagaggc tgaggggttt gtagtgtttt tgtgcaagga |
| 481 | caggtctgga gactgttctc ctgagaccag tttaaaacag ctgagactta aaagggatcc |
| 541 | tgggatagat ggtgttggtg aaatatcatc tcagttgatg ttcaccataa gccaagtcac |
| 601 | accgttgcac agtgggacct accagtgttg tgccagaagc cagaagtcag gtatccgcct |
| 661 | tcagggccat tttttctcca ttctattcac agagacaggg aactacacag tgacgggatt |
| 721 | gaaacaaaga caacaccttg agttcagcca taatgaaggc actctcagtt caggcttcct |
| 781 | acaagaaaag gtctgggtaa tgctggtcac cagccttgtg gcccttcaag ctttgtaagc |
| 841 | cttgtgccaa aagaaacttt taaaacagct acagcaagat gagtctgact atggcttagt |
| 901 | atctttctca ttacaatagg cacagagaag aatgcaacag ggcacagggg aagagatgct |
| 961 | aaatatacca agaatctgtg gaaatataag ctggggcaaa tcagtgtaat ccttgacttt |
| 1021 | gctcctcacc atcagggcaa acttgccttc ttccctccta agctccagta aataaacaga |
| 1081 | acagctttca ccaaagtggg tagtatagtc ctcaaatatc ggataaatat atgcgttttt |
| 1141 | gtaccccaga aaaacttttc ctccctcttc atcaacatag taaaataagt caaacaaaat |
| 1201 | gagaacacca aattttgggg gaataaattt ttatttaaca ctgcaaagga aagagagaga |
| 1261 | aaacaagcaa agataggtag gacagaaagg aagacagcca gatccagtga ttgacttggc |
| 1321 | atgaaaatga gaaaatgcag acagacctca acattcaaca acatccatac agcactgctg |
| 1381 | gaggaagagg aagatttgtg cagaccaaga gcaccacaga ctacaactgc ccagcttcat |
| 1441 | ctaaatactt gttaacctct ttggtcattt ctctttttaa ataaatgccc atagcagtat |
| 1501 | ttggagtctt ttcttttctc ctaaatccac aaactctctt ctttctcttt ggacagatga |
| 1561 | cctcttgtca tagttaagca gagagtgggc aggatattcc tgataggagg aactacatga |
| 1621 | ataaaggggt aag |

KLRF1 (SEQ ID NO: 33) - Homo sapiens killer cell lectin like receptor
F1 (KLRF1), transcript variant 1, mRNA - NM_016523

| | |
|---|---|
| 1 | atttcatgtt atacttaata aaacaaaaca tacctgtata cacacacatt cactcacatt |
| 61 | gaagatgcaa gatgaagaaa gatacatgac attgaatgta cagtcaaaga aaaggagttc |
| 121 | tgcccaaaca tctcaactta catttaaaga ttattcagtg acgttgcact ggtataaaat |
| 181 | cttactggga atatctggaa ccgtgaatgg tattctcact ttgactttga tctccttgat |
| 241 | cctgttggtt tctcagggag tattgctaaa atgccaaaaa ggaagttgtt caaatgccac |
| 301 | tcagtatgag gacactggag atctaaaagt gaataatggc acaagaagaa atataagtaa |
| 361 | taaggacctt tgtgcttcga gatctgcaga ccagacagta ctatgccaat cagaatggct |
| 421 | caaataccaa gggaagtgtt attggttctc taatgagatg aaaagctgga gtgacagtta |
| 481 | tgtgtattgt ttggaaagaa aatctcatct actaatcata catgaccaac ttgaaatggc |
| 541 | ttttatacag aaaaacctaa gacaattaaa ctacgtatgg attgggctta actttacctc |
| 601 | cttgaaaatg acatggactt gggtggatgg ttctccaata gattcaaaga tattcttcat |
| 661 | aaagggacca gctaaagaaa acagctgtgc tgccattaag gaaagcaaaa ttttctctga |
| 721 | aacctgcagc agtgttttca aatggatttg tcagtattag agtttgacaa aattcacagt |
| 781 | gaaataatca atgatcacta tttttggcct attagtttct aatattaatc tccaggtgta |
| 841 | agattttaaa gtgcaattaa atgccaaaat ctcttctccc ttctccctcc atcatcgaca |
| 901 | ctggtctagc ctcagagtaa cccctgttaa caaactaaaa tgtacacttc aaaattttta |
| 961 | cgtgatagta taaaccaatg tgacttcatg tgatcatatc caggattttt attcgtcgct |
| 1021 | tattttatgc caaatgtgat caaattatgc ctgtttttct gtatcttgcg ttttaaattc |
| 1081 | ttaataaggt cctaaacaaa atttcttata tttctaatgg ttgaattata atgtgggttt |
| 1141 | atacattttt tacccttttg tcaaagagaa ttaactttgt ttccaggctt ttgctactct |
| 1201 | tcactcagct acaataaaca tcctgaatgt tttcttaaaa aaaaaaaaaa aaaaaaaaaa |
| 1261 | aaaaaa |

CD2 (SEQ ID NO: 34) CD2 molecule, NM_001767.3

| | |
|---|---|
| 1 | agaatcaaaa gaggaaacca acccctaaga tgagctttcc atgtaaattt gtagccagct |
| 61 | tccttctgat tttcaatgtt tcttccaaag gtgcagtctc caaagagatt acgaatgcct |
| 121 | tggaaacctg gggtgccttg ggtcaggaca tcaacttgga cattcctagt tttcaaatga |
| 181 | gtgatgatat tgacgatata aaatgggaaa aaacttcaga caagaaaaag attgcacaat |
| 241 | tcagaaaaga gaaagagact ttcaaggaaa aagatacata taagctattt aaaaatggaa |
| 301 | ctctgaaaat taagcatctg aagaccgatg atcaggatat ctacaaggta tcaatatatg |
| 361 | atacaaaagg aaaaaatgtg ttggaaaaaa tatttgattt gaagattcaa gagagggtct |
| 421 | caaaaccaaa gatctcctgg acttgtatca acacaaccct gacctgtgag gtaatgaatg |
| 481 | gaactgaccc cgaattaaac ctgtatcaag atgggaaaca tctaaaactt tctcagaggg |
| 541 | tcatcacaca caagtggacc accagcctga gtgcaaaatt caagtgcaca gcagggaaca |
| 601 | aagtcagcaa ggaatccagt gtcgagcctg tcagctgtcc agagaaaggt ctggacatct |
| 661 | atctcatcat tggcatatgt ggaggaggca gcctcttgat ggtctttgtg gcactgctcg |
| 721 | ttttctatat caccaaaagg aaaaaacaga ggagtcggag aaatgatgag gagctggaga |
| 781 | caagagccca cagagtagct actgaagaaa ggggccggaa gccccaccaa attccagctt |
| 841 | caacccctca gaatccagca acttcccaac atcctcctcc accacctggt catcgttccc |
| 901 | aggcacctag tcatcgtccc ccgcctcctg gacaccgtgt tcagcaccag cctcagaaga |
| 961 | ggcctcctgc tccgtcgggc acacaagttc accagcagaa aggcccgccc ctccccagac |
| 1021 | ctcgagttca gccaaaacct ccccatgggg cagcagaaaa ctcattgtcc ccttcctcta |
| 1081 | attaaaaaag atagaaactg tctttttcaa taaaaagcac tgtggatttc tgccctcctg |
| 1141 | atgtgcatat ccgtacttcc atgaggtgtt ttctgtgtgc agaacattgt cacctcctga |
| 1201 | ggctgtgggc cacagccacc tctgcatctt cgaactcagc catgtggtca acatctggag |
| 1261 | tttttggtct cctcagagag ctccatcaca ccagtaagga gaagcaatat aagtgtgatt |
| 1321 | gcaagaatgg tagaggaccg agcacagaaa tcttagagat ttcttgtccc ctctcaggtc |
| 1381 | atgtgtagat gcgataaatc aagtgattgg tgtgcctggg tctcactaca agcagcctat |
| 1441 | ctgcttaaga gactctggag tttcttatgt gccctggtgg acacttgccc accatcctgt |
| 1501 | gagtaaaagt gaaataaaag ctttgactag aaaaaaaaaa aaaaaaaaaa aaaaaaaaaa |
| 1561 | aaaaaaaaaa aaaaaaaaaa aaaaaaaaaa aaaaa |

LGALS2 (SEQ ID NO: 35) - Homo sapiens lectin, galactoside-binding,
soluble, 2 (LGALS2), mRNA - NM_006498

| | |
|---|---|
| 1 | gaccttgagg gagttaatgt gtaatattct aggatataag cttgaccacg agttgagacc |
| 61 | ctgagcacag gcctccagga gccgctggga gctgccgcca ggagctgtca ccatgacggg |
| 121 | ggaacttgag gttaagaaca tggacatgaa gccggggtca accctgaaga tcacaggcag |
| 181 | catcgccgat ggcactgatg gctttgtaat taatctgggc caggggacag acaagctgaa |
| 241 | cctgcatttc aaccctcgct tcagcgaatc caccattgtc tgcaactcat tggacggcag |
| 301 | caactggggg caagaacaac gggaagatca cctgtgcttc agcccagggt cagaggtcaa |
| 361 | gttcacagtg acctttgaga gtgacaaatt caaggtgaag ctgccagatg ggcacgagct |
| 421 | gacttttccc aacaggctgg gtcacagcca cctgagctac ctgagcgtaa ggggcgggtt |
| 481 | caacatgtcc tctttcaagt taaaagaata aaagacttcc agccgagaaa aaaaaaaaaa |
| 541 | aaa |

NPPC (SEQ ID NO: 36) - Homo sapiens natriuretic peptide C, mRNA -
NM_024409

| | |
|---|---|
| 1 | cgggctcaga gcgcacccag ccggcgccgc gcagcactgg gaccctgctc gccctgcagc |
| 61 | ccagccagcc tgctccgcat ccccctgctg gtctgcccgc cgacctgcgc gccctcgctg |
| 121 | ccgcccgtgt gcgcccctcg accccagcgg caccatgcat ctctcccagc tgctggcctg |
| 181 | cgccctgctg ctcacgctgc tctccctccg gccctccgaa gccaagcccg gggcgccgcc |
| 241 | gaaggtcccg cgaaccccgc cggcagagga gctggccgag ccgcaggctg cgggcggcgg |
| 301 | tcagaagaag ggcgacaagg ctcccggggg cgggggcgcc aatctcaagg gcgaccggtc |
| 361 | gcgactgctc cgggacctgc gcgtggacac caagtcgcgg gcagcgtggg ctcgccttct |
| 421 | gcaagagcac cccaacgcgc gcaaatacaa aggagccaac aagaagggct tgtccaaggg |
| 481 | ctgcttcggc ctcaagctgg accgaatcgg ctccatgagc ggcctgggat gttagtgcgg |
| 541 | cgccccctgg cggcggatcg ggaactggct ccgttgtgct gaggtcatct ttggtcatca |
| 601 | gcctccagca tctggaaaca cctccaacgc aatgtggctt ttacatttct ttctttcttt |
| 661 | cttttttttt cctggtactg ggaatacaca acaccagctg ttttattatt atttggggag |
| 721 | ggggttgtga ttttattatt tgttttttta aaatgaaaaa taaaaagtta tatatta |

MYCL (SEQ ID NO: 37) - Homo sapiens v-myc avian myelocytomatosis
viral oncogene lung carcinoma derived homolog (MYCL), transcript
variant 1, mRNA - NM_001033081

| | |
|---|---|
| 1 | aatgcgcctg cagctcgcgc tcccgcgccg atcccgagag cgtccgggcc gccgtgcgcg |
| 61 | agcgagggag ggcgcgcgcg cggggggggc gcgcttgtga gtgcgggccg cgctctcggc |
| 121 | ggcgcgcatg tgcgtgtgtg ctggctgccg ggctgccccg agccggcggg gagccggtcc |
| 181 | gctccaggtg gcgggcggct ggagcgaggt gaggctgcgg gtggccaggg cacgggcgcg |
| 241 | ggtcccgcgg tgcgggctgg ctgcaggctg ccttctgggc acggcgcgcc cccgcccggc |
| 301 | cccgccgggc cctgggagct gcgctccggg cggcgctggc aaagtttgct ttgaactcgc |
| 361 | tgcccacagt cgggtccgcg cgctgcgatt ggcttcccct accactctga cccggggccc |
| 421 | ggcttcccgg gacgcgagga ctgggcgcag gctgcaagct ggtggggttg gggaggaacg |
| 481 | agagcccggc agccgactgt gccgagggac ccggggacac ctccttcgcc cggccggcac |
| 541 | ccggtcagca cgtcccccct tccctcccgc agggagcgga catggactac gactcgtacc |
| 601 | agcactattt ctacgactat gactgcgggg aggatttcta ccgctccacg gcgcccagcg |
| 661 | aggacatctg gaagaaattc gagctggtgc catcgccccc cacgtcgccg ccctggggct |
| 721 | tgggtcccgg cgcaggggac ccggcccccg ggattggtcc cccggagccg tggcccggag |
| 781 | ggtgcaccgg agacgaagcg gaatcccggg gccactcgaa aggctggggc aggaactacg |
| 841 | cctccatcat acgccgtgac tgcatgtgga gcggcttctc ggcccgggaa cggctggaga |
| 901 | gagctgtgag cgaccggctc gctcctggcg cgccccgggg gaacccgccc aaggcgtccg |
| 961 | ccgccccgga ctgcactccc agcctcgaag ccggcaaccc ggcgcccgcc gccccctgtc |
| 1021 | cgctgggcga acccaagacc caggcctgct ccgggtccga gagcccaagc gactcggaga |
| 1081 | atgaagaaat tgatgttgtg acagtagaga agaggcagtc tctgggtatt cggaagccgg |
| 1141 | tcaccatcac ggtgcgagca gaccccctgg atccctgcat gaagcatttc cacatctcca |
| 1201 | tccatcagca acagcacaac tatgctgccc gttttcctcc agaaagctgc tcccaagaag |
| 1261 | aggcttcaga gaggggtccc caagaagagg ttctggagag agatgctgca ggggaaaagg |
| 1321 | aagatgagga ggatgaagag attgtgagtc ccccacctgt agaaagtgag gctgcccagt |
| 1381 | cctgccaccc caaacctgtc agttctgata ctgaggatgt gaccaagagg aagaatcaca |
| 1441 | acttcctgga gcgcaagagg cggaatgacc tgcgttcgcg attcttggcg ctgagggacc |
| 1501 | aggtgcccac cctggccagc tgctccaagg cccccaaagt agtgatccta agcaaggcct |
| 1561 | tggaatactt gcaagccctg gtgggggctg agaagaggat ggctacagag aaaagacagc |
| 1621 | tccgatgccg gcagcagcag ttgcagaaaa gaattgcata cctcactggc tactaactga |
| 1681 | ccaaaaagcc tgacagttct gtcttacgaa gacacaagtt tattttttaa cctccctctc |
| 1741 | ccctttagta atttgcacat tttggttatg gtgggacagt ctggacagta gatcccagaa |
| 1801 | tgcattgcag ccggtgcaca cacaataaag gcttgcattc ttggaaacct tgaaacccag |
| 1861 | ctctccctct tccctgactc atgggagtgc tgtatgttct ctggcgcctt tggcttccca |
| 1921 | gcaggcagct gactgaggag ccttggggtc tgcctagctc actagctctg aagaaaaggc |
| 1981 | tgacagatgc tatgcaacag gtggtggatg ttgtcagggg ctccagcctg catgaaatct |
| 2041 | cacactctgc atgagcttta ggctaggaaa ggatgctccc aactggtgtc tctggggtga |
| 2101 | tgcaaggaca gctgggcctg gatgctctcc ctgaggctcc tttttccaga agacacacga |
| 2161 | gctgtcttgg gtgaagacaa gcttgcagac ttgatcaaca ttgaccatta cctcactgtc |
| 2221 | agacacttta cagtagccaa ggagttggaa acctttatat attatgatgt tagctgaccc |
| 2281 | ccttcctccc actcccaatg ctgcgaccct gggaacactt aaaaagcttg gcctctagat |
| 2341 | tctttgtctc agagccctct gggctctctc ctctgaggga gggacctttc tttcctcaca |
| 2401 | agggactttt ttgttccatt atgccttgtt atgcaatggg ctctacagca ccctttccca |
| 2461 | caggtcagaa atatttcccc aagacacagg gaaatcggtc ctagcctggg gcctggggat |
| 2521 | agcttggagt cctggcccat gaacttgatc cctgcccagg tgttttccga ggggcacttg |
| 2581 | aggcccagtc ttttctcaag gcaggtgtaa gacacctcag agggagaact gtactgctgc |
| 2641 | ctctttccca cctgcctcat ctcaatcctt gagcggcaag tttgaagttc ttctggaacc |
| 2701 | atgcaaatct gtcctcctca tgcaattcca aggagcttgc tggctctgca gccacccttg |
| 2761 | ggccccttcc agcctgccat gaatcagata tctttcccag aatctgggcg tttctgaagt |
| 2821 | tttggggaga gctgttggga ctcatccagt gctccagaag gtggacttgc ttctggtggg |
| 2881 | ttttaaagga gcctccagga gatatgctta gccaaccatg atggatttta ccccagctgg |
| 2941 | actcggcagc tccaagtgga atccacgtgc agcttctagt ctgggaaagt cacccaacct |
| 3001 | agcagttgtc atgtgggtaa cctcaggcac ctctaagcct gtcctggaag aaggaccagc |
| 3061 | agcccctcca gaactctgcc caggacagca ggtgcctgct ggctctgggt ttggaagttg |
| 3121 | gggtgggtag ggggtggtaa gtactatata tggctctgga aaaccagctg ctacttccaa |
| 3181 | atctattgtc cataatggtt tctttctgag gttgcttctt ggcctcagag gaccccaggg |
| 3241 | gatgtttgga aatagcctct ctacccttct ggagcatggt ttacaaaagc cagctgactt |
| 3301 | ctggaattgt ctatggagga cagtttgggt gtaggttact gatgtctcaa ctgaatagct |
| 3361 | tgtgttttat aagctgctgt tggctattat gctgggggag tctttttttt ttatattgta |
| 3421 | tttttgtatg ccttttgcaa agtggtgtta actgtttttg tacaaggaaa aaaactcttg |
| 3481 | gggcaatttc ctgttgcaag ggtctgattt attttgaaag gcaagttcac ctgaaatttt |
| 3541 | gtatttagtt gtgattactg attgcctgat tttaaaatgt tgccttctgg gacatcttct |
| 3601 | aataaaagat ttctcaaaca tgtc |

MYCL (SEQ ID NO: 38) - Homo sapiens v-myc avian myelocytomatosis
viral oncogene lung carcinoma derived homolog (MYCL), transcript
variant 3, mRNA - NM_005476

| | |
|---|---|
| 1 | aatgcgcctg cagctcgcgc tcccgcgccg atcccgagag cgtccgggcc gccgtgcgcg |
| 61 | agcgagggag ggcgcgcgcg cggggggggc gcgcttgtga gtgcgggccg cgctctcggc |
| 121 | ggcgcgcatg tgcgtgtgtg ctggctgccg ggctgccccg agccggcggg gagccggtcc |
| 181 | gctccaggtg gcgggcggct ggagcgaggg agcggacatg gactacgact cgtaccagca |
| 241 | ctatttctac gactatgact gcggggagga tttctaccgc tccacggcgc ccagcgagga |
| 301 | catctggaag aaattcgagc tggtgccatc gccccccacg tcgccgccct ggggcttggg |
| 361 | tcccggcgca ggggacccgg cccccgggat tggtcccccg gagccgtggc ccggagggtg |
| 421 | caccggagac gaagcggaat cccggggcca ctcgaaaggc tggggcagga actacgcctc |
| 481 | catcatacgc cgtgactgca tgtggagcgg cttctcggcc cgggaacggc tggagagagc |
| 541 | tgtgagcgac cggctcgctc ctggcgcgcc ccgggggaac ccgcccaagg cgtccgccgc |
| 601 | cccggactgc actcccagcc tcgaagccgg caacccggcg cccgccgccc cctgtccgct |
| 661 | gggcgaaccc aagacccagg cctgctccgg gtccgagagc ccaagcgact cgggtaagga |
| 721 | cctccccgag ccatccaaga gggggccacc ccatgggtgg ccaaagctct gcccctgcct |
| 781 | gaggtcaggc attggctctt ctcaagctct tgggccatct ccgcctctct ttggctgaag |
| 841 | ctgcccgtgt agtccccaac cgtgtctgtc tggcacgtgg gtgtgttggt aaacagtttg |
| 901 | gaaaagtggc gtgggagcca gcctcccttt gatgattatt ggagccccag gggacaaggg |
| 961 | atttgaggtg agggttggcg cttagagagg acaatactgg ggttggactg taagggattg |
| 1021 | aagggggtac cttaagagac actccaaacc tgaagttttt ttgctgctgc ctctttccct |
| 1081 | aggaaactca cactccccta gggggagaag aagccgagag ccttttgtgc aaagccaaaa |
| 1141 | ccttcgtcct tttaaaaacc taggtctcca gttggcttta ctttaaaatg ccaataataa |
| 1201 | atgccctctt ctcgtgcctc cccaccacca cttaccactc gtgcatccct gagacaggga |
| 1261 | gggaagaatg aacactcccc attaacagat ggaaaaactg aggcttagag atagacaatc |
| 1321 | actacaagtc agctccagct ttctgccatc tagccagccc ctcttcccca atgctccatc |
| 1381 | ccaaccaggc acctcttcct tgatgtttgg ggtctttgtg gtagcttatc ttagaagcac |
| 1441 | tacaccttgc cttgctgttt gtcctgagat ggaaaagtgt ccttcttgct ccccctcaat |
| 1501 | agatctccag cgtcagctgc tccctggcat tcaacaaata ttcactggcc cctactttgt |
| 1561 | ggcaatctgt gggctacatg ctggggtcaa ggcagtagaa ctccaggccc tcctctccca |
| 1621 | tccttgatgc aagtgcaacc tcgctgaggg cagactgggg catcctgtgc cactaaacta |
| 1681 | cattgttctt attctggcat cttagacctc cacacccgtg agaaatcctg gagagggtat |
| 1741 | ttttgtagag tgtagactgt ggctagtgac aaataaatta ggaccaagaa agctcactgt |
| 1801 | agcttttagg aataactttt acacgaccat ttgataggga actggggaat ggggtatgga |
| 1861 | agttttccta cacttgagag aaaaaatagg ataacaaaaa ttaaaagtct tttttttcct |
| 1921 | ggtccactgt gttaaggtca tttttaacca gcttgctttc tacaccaaga gtttatgttt |
| 1981 | gtttaatggc tggaaagaga atcttgagat caaaaaacca ataaagatgt atctctacaa |
| 2041 | aaaaaaaaaa |

MX1 (SEQ ID NO: 39) MX dynamin like G7Pase 1, transcript variant 1,
NM_001144925.2

| | |
|---|---|
| 1 | cgagcagaaa tgaaaccgaa actgaattgt ccgggaaatt cgcggtgggg gcggagagcg |
| 61 | cagggagaag taagcccagt gcaggatcct gaggcccgtg tttgcaggac cagggccggc |
| 121 | cttccgattc cccattcatt ccagaagcac cgaaccacgc tgtgcccgga tcccaagtgc |
| 181 | agcggcaccc agcgtgggcc tggggttgcc ggttgacccg gtcctcagcc tggtagcaga |
| 241 | ggccaggcca gtgccacaag gcacctaagt ccacctgggc ctggagcagg acaggttgca |
| 301 | aaagaaaata tctcgggacc cccaaactcc ttatgctaag ggaaacatcg agcctgggaa |
| 361 | ctgagccatc aacgctgcca ttctttttcc caaacagaac cctgttgtca gaggtacacc |
| 421 | cagagcaact ccacaccggg tgcatgccac agcaactcca tcttaaatag gagctggtaa |
| 481 | aacgaggctg atacctactg ggctgcattc ccagacggca tagcgaggag gtgctgaaga |
| 541 | gcgcaggttt ggagaatgat cacctggatt ggaaccatag ctctaccaat atggaaccca |
| 601 | gctccttagg cctcggtctt ctcatggaga acatggtgtg ataatcctac tcctctggga |
| 661 | gggtggctgt taagccttgg accgcagttg ccggccagga atcccagtgt cacggtggac |
| 721 | acgcctccct cgcgcccttg ccgcccacct gctcacccag ctcaggggct ttggaattct |
| 781 | gtggccacac tgcgaggaga tcggttctgg gtcggaggct acaggaagac tcccactccc |
| 841 | tgaaatctgg agtgaagaac gccgccatcc agccaccatt ccaaggaggt gcaggagaac |
| 901 | agctctgtga taccatttaa cttgttgaca ttacttttat ttgaaggaac gtatattaga |
| 961 | gcttactttg caaagaagga agatggttgt ttccgaagtg gacatcgcaa aagctgatcc |
| 1021 | agctgctgca tcccaccctc tattactgaa tggagatgct actgtggccc agaaaaatcc |
| 1081 | aggctcggtg gctgagaaca acctgtgcag ccagtatgag gagaaggtgc gcccctgcat |
| 1141 | cgacctcatt gactccctgc gggctctagg tgtggagcag gacctggccc tgccagccat |
| 1201 | cgccgtcatc ggggaccaga gctcgggcaa gagctccgtg ttggaggcac tgtcaggagt |
| 1261 | tgcccttccc agaggcagcg ggatcgtgac cagatgcccg ctggtgctga aactgaagaa |
| 1321 | acttgtgaac gaagataagt ggagaggcaa ggtcagttac caggactacg agattgagat |
| 1381 | ttcggatgct tcagaggtag aaaaggaaat taataaagcc cagaatgcca tcgccgggga |
| 1441 | aggaatggga atcagtcatg agctaatcac cctggagatc agctcccgag atgtcccgga |
| 1501 | tctgactcta atagaccttc ctggcataac cagagtggct gtgggcaatc agcctgctga |
| 1561 | cattgggtat aagatcaaga cactcatcaa gaagtacatc cagaggcagg agacaatcag |
| 1621 | cctggtggtg gtccccagta atgtggacat cgccaccaca gaggctctca gcatggccca |
| 1681 | ggaggtggac cccgagggag acaggaccat cggaatcttg acgaagcctg atctggtgga |
| 1741 | caaaggaact gaagacaagg ttgtggacgt ggtgcggaac ctcgtgttcc acctgaagaa |
| 1801 | gggttacatg attgtcaagt gccggggcca gcaggagatc caggaccagc tgagcctgtc |
| 1861 | cgaagccctg cagagagaga agatcttctt tgagaaccac ccatatttca gggatctgct |
| 1921 | ggaggaagga aaggccacgg ttccctgcct ggcagaaaaa cttaccagcg agctcatcac |
| 1981 | acatatctgt aaatctctgc ccctgttaga aaatcaaatc aaggagactc accagagaat |
| 2041 | aacagaggag ctacaaaagt atggtgtcga cataccggaa gacgaaaatg aaaaaatgtt |
| 2101 | cttcctgata gataaagtta atgcctttaa tcaggacatc actgctctca tgcaaggaga |
| 2161 | ggaaactgta ggggaggaag acattcggct gtttaccaga ctccgacacg agttccacaa |
| 2221 | atggagtaca ataattgaaa acaattttca agaaggccat aaaattttga gtagaaaaat |
| 2281 | ccagaaattt gaaaatcagt atcgtggtag agagctgcca ggctttgtga attacaggac |
| 2341 | atttgagaca atcgtgaaac agcaaatcaa ggcactggaa gagccggctg tggatatgct |
| 2401 | acacaccgtg acggatatgg tccggcttgc tttcacagat gtttcgataa aaaattttga |
| 2461 | agagtttttt aacctccaca gaaccgccaa gtccaaaatt gaagacatta gagcagaaca |
| 2521 | agagagagaa ggtgagaagc tgatccgcct ccacttccag atggaacaga ttgtctactg |
| 2581 | ccaggaccag gtatacaggg gtgcattgca gaaggtcaga gagaaggagc tggaagaaga |
| 2641 | aaagaagaag aaatcctggg attttggggc tttccagtcc agctcggcaa cagactcttc |
| 2701 | catggaggag atctttcagc acctgatggc ctatcaccag gaggccagca agcgcatctc |
| 2761 | cagccacatc cctttgatca tccagttctt catgctccag acgtacggcc agcagcttca |
| 2821 | gaaggccatg ctgcagctcc tgcaggacaa ggacacctac agctggctcc tgaaggagcg |
| 2881 | gagcgacacc agcgacaagc ggaagttcct gaaggagcgg cttgcacggc tgacgcaggc |
| 2941 | tcggcgccgg cttgcccagt tccccggtta accacactct gtccagcccc gtagacgtgc |
| 3001 | acgcacactg tctgcccccg ttcccgggta gccactggac tgacgacttg agtgctcagt |
| 3061 | agtcagactg gatagtccgt ctctgcttat ccgttagccg tggtgattta gcaggaagct |
| 3121 | gtgagagcag tttggtttct agcatgaaga cagagcccca ccctcagatg cacatgagct |
| 3181 | ggcgggattg aaggatgctg tcttcgtact gggaaaggga ttttcagccc tcagaatcgc |
| 3241 | tccaccttgc agctctcccc ttctctgtat tcctagaaac tgacacatgc tgaacatcac |
| 3301 | agcttatttc ctcattttta taatgtccct tcacaaaccc agtgttttag gagcatgagt |
| 3361 | gccgtgtgtg tgcgtcctgt cggagccctg tctcctctct ctgtaataaa ctcatttcta |
| 3421 | gcagacaaaa aaaaaaaaaa aaaa |

CCL5 (SEQ ID NO: 40) - Homo sapiens C-C motif chemokine ligand 5
(CCL5), transcript variant 1, mRNA - NM_002985

| | |
|---|---|
| 1 | gctgcagagg attcctgcag aggatcaaga cagcacgtgg acctcgcaca gcctctccca |
| 61 | caggtaccat gaaggtctcc gcggcagccc tcgctgtcat cctcattgct actgccctct |
| 121 | gcgctcctgc atctgcctcc ccatattcct cggacaccac accctgctgc tttgcctaca |
| 181 | ttgcccgccc actgccccgt gcccacatca aggagtattt ctacaccagt ggcaagtgct |
| 241 | ccaacccagc agtcgtcttt gtcacccgaa agaaccgcca agtgtgtgcc aacccagaga |
| 301 | agaaatgggt tcgggagtac atcaactctt tggagatgag ctaggatgga gagtccttga |
| 361 | acctgaactt acacaaattt gcctgtttct gcttgctctt gtcctagctt gggaggcttc |
| 421 | ccctcactat cctaccccac ccgctccttg aagggcccag attctaccac acagcagcag |
| 481 | ttacaaaaac cttccccagg ctggacgtgg tggctcacgc ctgtaatccc agcactttgg |
| 541 | gaggccaagg tgggtggatc acttgaggtc aggagttcga gaccagcctg gccaacatga |
| 601 | tgaaacccca tctctactaa aaatacaaaa aattagccgg gcgtggtagc gggcgcctgt |
| 661 | agtcccagct actcgggagg ctgaggcagg agaatggcgt gaacccggga ggcggagctt |
| 721 | gcagtgagcc gagatcgcgc cactgcactc cagcctgggc gacagagcga gactccgtct |
| 781 | caaaaaaaaa aaaaaaaaaa aaaatacaaa aattagccgg gcgtggtggc ccacgcctgt |
| 841 | aatcccagct actcgggagg ctaaggcagg aaaattgttt gaacccagga ggtggaggct |
| 901 | gcagtgagct gagattgtgc cacttcactc cagcctgggt gacaaagtga gactccgtca |
| 961 | caacaacaac aacaaaaagc ttccccaact aaagcctaga agagcttctg aggcgctgct |
| 1021 | ttgtcaaaag gaagtctcta ggttctgagc tctggctttg ccttggcttt gccagggctc |
| 1081 | tgtgaccagg aaggaagtca gcatgcctct agaggcaagg aggggaggaa cactgcactc |
| 1141 | ttaagcttcc gccgtctcaa cccctcacag gagcttactg gcaaacatga aaaatcggct |
| 1201 | taccattaaa gttctcaatg caaccataaa aaaaaaa |

TGFBI (SEQ ID NO: 41) - Homo sapiens transforming growth factor beta
induced (TGFBI), mRNA - NM_000358

| | |
|---|---|
| 1 | ctccttgcac gggccggccc agcttccccg cccctggcgt ccgctccctc ccgctcgcag |
| 61 | cttacttaac ctggcccggg cggcggaggc gctctcactt ccctggagcc gcccgcttgc |
| 121 | ccgtcggtcg ctagctcgct cggtgcgcgt cgtcccgctc catggcgctc ttcgtgcggc |
| 181 | tgctggctct cgccctggct ctggccctgg gccccgccgc gaccctggcg ggtcccgcca |
| 241 | agtcgcccta ccagctggtg ctgcagcaca gcaggctccg gggccgccag cacggcccca |
| 301 | acgtgtgtgc tgtgcagaag gttattggca ctaataggaa gtacttcacc aactgcaagc |
| 361 | agtggtacca aaggaaaatc tgtggcaaat caacagtcat cagctacgag tgctgtcctg |
| 421 | gatatgaaaa ggtccctggg gagaagggct gtccagcagc cctaccactc tcaaaccttt |
| 481 | acgagaccct gggagtcgtt ggatccacca ccactcagct gtacacggac cgcacggaga |
| 541 | agctgaggcc tgagatggag gggcccggca gcttcaccat cttcgcccct agcaacgagg |
| 601 | cctgggcctc cttgccagct gaagtgctgg actccctggt cagcaatgtc aacattgagc |
| 661 | tgctcaatgc cctccgctac catatggtgg gcaggcgagt cctgactgat gagctgaaac |
| 721 | acggcatgac cctcacctct atgtaccaga attccaacat ccagatccac cactatccta |
| 781 | atgggattgt aactgtgaac tgtgcccggc tgctgaaagc cgaccaccat gcaaccaacg |
| 841 | gggtggtgca cctcatcgat aaggtcatct ccaccatcac caacaacatc cagcagatca |
| 901 | ttgagatcga ggacaccttt gagacccttc gggctgctgt ggctgcatca gggctcaaca |
| 961 | cgatgcttga aggtaacggc cagtacacgc ttttggcccc gaccaatgag gccttcgaga |
| 1021 | agatccctag tgagactttg aaccgtatcc tgggcgaccc agaagccctg agagacctgc |
| 1081 | tgaacaacca catcttgaag tcagctatgt gtgctgaagc catcgttgcg gggctgtctg |
| 1141 | tagagaccct ggagggcacg acactggagg tgggctgcag cggggacatg ctcactatca |
| 1201 | acgggaaggc gatcatctcc aataaagaca tcctagccac caacggggtg atccactaca |
| 1261 | ttgatgagct actcatccca gactcagcca agacactatt tgaattggct gcagagtctg |
| 1321 | atgtgtccac agccattgac cttttcagac aagccggcct cggcaatcat ctctctggaa |
| 1381 | gtgagcggtt gaccctcctg gctcccctga attctgtatt caaagatgga acccctccaa |
| 1441 | ttgatgccca tacaaggaat ttgcttcgga accacataat taaagaccag ctggcctcta |
| 1501 | agtatctgta ccatggacag accctggaaa ctctgggcgg caaaaaactg agagtttttg |
| 1561 | tttatcgtaa tagcctctgc attgagaaca gctgcatcgc ggcccacgac aagaggggga |
| 1621 | ggtacgggac cctgttcacg atggaccggg tgctgacccc cccaatgggg actgtcatgg |
| 1681 | atgtcctgaa gggagacaat cgctttagca tgctggtagc tgccatccag tctgcaggac |
| 1741 | tgacggagac cctcaaccgg gaaggagtct acacagtctt tgctcccaca aatgaagcct |
| 1801 | tccgagccct gccaccaaga gaacggagca gactcttggg agatgccaag gaacttgcca |
| 1861 | acatcctgaa ataccacatt ggtgatgaaa tcctggttag cggaggcatc ggggccctgg |
| 1921 | tgcggctaaa gtctctccaa ggtgacaagc tggaagtcag cttgaaaaac aatgtggtga |
| 1981 | gtgtcaacaa ggagcctgtt gccgagcctg acatcatggc cacaaatggc gtggtccatg |
| 2041 | tcatcaccaa tgttctgcag cctccagcca acagacctca ggaaagaggg gatgaacttg |
| 2101 | cagactctgc gcttgagatc ttcaaacaag catcagcgtt ttccagggct tcccagaggt |
| 2161 | ctgtgcgact agcccctgtc tatcaaaagt tattagagag gatgaagcat tagcttgaag |
| 2221 | cactacagga ggaatgcacc acggcagctc tccgccaatt tctctcagat ttccacagag |
| 2281 | actgtttgaa tgttttcaaa accaagtatc acactttaat gtacatgggc cgcaccataa |
| 2341 | tgagatgtga gccttgtgca tgtgggggag gagggagaga gatgtacttt ttaaatcatg |
| 2401 | ttccccctaa acatggctgt taacccactg catgcagaaa cttggatgtc actgcctgac |
| 2461 | attcacttcc agagaggacc tatcccaaat gtggaattga ctgcctatgc caagtccctg |
| 2521 | gaaaaggagc ttcagtattg tggggctcat aaaacatgaa tcaagcaatc cagcctcatg |
| 2581 | ggaagtcctg gcacagtttt tgtaaagccc ttgcacagct ggagaaatgg catcattata |
| 2641 | agctatgagt tgaaatgttc tgtcaaatgt gtctcacatc tacacgtggc ttggaggctt |
| 2701 | ttatggggcc ctgtccaggt agaaaagaaa tggtatgtag agcttagatt tccctattgt |
| 2761 | gacagagcca tggtgtgttt gtaataataa aaccaaagaa acata |

PLA2G7 (SEQ ID NO: 42) - Homo sapiens phospholipase A2 group VII
(PLA2G7), transcript variant 1, mRNA - NM_005084

| | |
|---|---|
| 1 | gggtcggggc cacaaggccg cgctaggcgg acccaggaca cagcccgcgc gcagcccacc |
| 61 | cgcccgccgc ctgccagagc tgctcggccc gcagccaggg ggacagcggc tggtcggagg |
| 121 | ctcgcagtgc tgtcggcgag aagcagtcgg gtttggagcg cttgggtcgc gttggtgcgc |
| 181 | ggtggaacgc gcccagggac cccagttccc gcgagcagct ccgcgccgcg cctgagagac |
| 241 | taagctgaaa ctgctgctca gctcccaaga tggtgccacc caaattgcat gtgcttttct |
| 301 | gcctctgcgg ctgcctggct gtggtttatc cttttgactg gcaatacata aatcctgttg |
| 361 | cccatatgaa atcatcagca tgggtcaaca aaatacaagt actgatggct gctgcaagct |
| 421 | ttggccaaac taaaatcccc cggggaaatg ggccttattc cgttggttgt acagacttaa |
| 481 | tgtttgatca cactaataag ggcaccttct tgcgtttata ttatccatcc caagataatg |
| 541 | atcgccttga caccctttgg atcccaaata aagaatattt ttggggtctt agcaaatttc |
| 601 | ttggaacaca ctggcttatg ggcaacattt tgaggttact ctttggttca atgacaactc |
| 661 | ctgcaaactg gaattcccct ctgaggcctg gtgaaaaata tccacttgtt gttttttctc |
| 721 | atggtcttgg ggcattcagg acactttatt ctgctattgg cattgacctg gcatctcatg |
| 781 | ggtttatagt tgctgctgta gaacacagag atagatctgc atctgcaact tactatttca |
| 841 | aggaccaatc tgctgcagaa ataggggaca agtcttggct ctaccttaga accctgaaac |
| 901 | aagaggagga gacacatata cgaaatgagc aggtacggca aagagcaaaa gaatgttccc |
| 961 | aagctctcag tctgattctt gacattgatc atggaaagcc agtgaagaat gcattagatt |
| 1021 | taaagtttga tatggaacaa ctgaaggact ctattgatag ggaaaaaata gcagtaattg |
| 1081 | gacattcttt tggtggagca acggttattc agactcttag tgaagatcag agattcagat |
| 1141 | gtggtattgc cctggatgca tggatgtttc cactgggtga tgaagtatat tccagaattc |
| 1201 | ctcagcccct cttttttatc aactctgaat atttccaata tcctgctaat atcataaaaa |
| 1261 | tgaaaaaatg ctactcacct gataaagaaa gaaagatgat tacaatcagg ggttcagtcc |
| 1321 | accagaattt tgctgacttc acttttgcaa ctggcaaaat aattggacac atgctcaaat |
| 1381 | taaagggaga catagattca aatgtagcta ttgatcttag caacaaagct tcattagcat |
| 1441 | tcttacaaaa gcatttagga cttcataaag attttgatca gtgggactgc ttgattgaag |
| 1501 | gagatgatga gaatcttatt ccagggacca acattaacac aaccaatcaa cacatcatgt |
| 1561 | tacagaactc ttcaggaata gagaaataca attaggatta aaataggttt tttaaaagtc |
| 1621 | ttgtttcaaa actgtctaaa attatgtgtg tgtgtgtgtg tgtgtgtgtg tgtgtgagag |
| 1681 | agagagagag agagagagag agagagagag agaattttaa tgtattttcc caaaggactc |
| 1741 | atattttaaa atgtaggcta tactgtaatc gtgattgaag cttggactaa gaattttttc |
| 1801 | cctttagatg taaagaaaga atacagtata caatattcaa aaaaaaaaaa aaaaaaaaaa |
| 1861 | aaaaaaaaaa aaaaaaaaaa |

ARHGEF10L (SEQ ID NO: 43) - Homo sapiens Rho guanine nucleotide
exchange factor 10 like (ARHGEF10L), transcript variant 1, mRNA -
NM_018125

| | |
|---|---|
| 1 | gcgccgtccc ggccatgggc gcccgcggcg gcctgcggag ctggaggcgc ggcgccggcc |
| 61 | gccaggcgcc tttgtgagcg gcgcggacga caaaggcgcg ggcccgggca gccgaggtgt |
| 121 | gtagctggga cggtgctggt ctgagctgga ccttgtctga tggcttcctc caaccctcct |
| 181 | ccacagcctg ccataggaga tcagctggtt ccaggagtcc caggcccctc ctctgaggca |
| 241 | gaggacgacc caggagaggc gtttgagttt gatgacagtg atgatgaaga ggacaccagc |
| 301 | gcagccctgg gcgtccccag ccttgctcct gagagggaca cagacccccc actgatccac |
| 361 | ttggactcca tccctgtcac tgacccagac ccagcagctg ctccacccgg cacaggggtg |
| 421 | ccagcctggg tgagcaatgg ggatgcagcg gacgcagcct tctccggggc ccggcactcc |
| 481 | agctggaagc ggaagagttc ccgtcgcatt gaccggttca ctttccccgc cctggaagag |
| 541 | gatgtgattt atgacgacgt cccctgcgag agcccagatg cgcatcagcc cggggcagag |
| 601 | aggaacctgc tctacgagga tgcgcaccgg gctggggccc ctcggcaggc ggaggaccta |
| 661 | ggctggagct ccagtgagtt cgagagctac agcgaggact cgggggagga ggccaagccg |
| 721 | gaggtcgagg tcgagcccgc caagcaccga gtgtccttcc agcccaagct ttctccagac |
| 781 | ctgactaggc taaaggagag atacgccagg actaagagag acatcttggc tttgagagtt |
| 841 | ggggggagag acatgcagga gctgaagcac aagtacgatt gtaagatgac ccagctcatg |
| 901 | aaggccgcca agagcgggac caaggatggg ctggagaaga cacggatggc cgtgatgcgc |
| 961 | aaagtctcct tcctgcacag gaaggacgtc ctcggtgact cggaggagga ggacatgggg |
| 1021 | ctcctggagg tcagcgtttc ggacatcaag cccccagccc cagagctggg ccccatgcca |
| 1081 | gagggcctga gccctcagca ggtggtccgg aggcatatcc tgggctccat cgtgcagagc |
| 1141 | gaaggcagct acgtggagtc tctgaagcgg atactccagg actaccgcaa ccccctgatg |
| 1201 | gagatggagc ccaaggcgct gagcgcccgc aagtgccagg tggtgttctt ccgcgtgaag |
| 1261 | gagatcctgc actgccactc catgttccag atcgccctgt cctcccgcgt ggctgagtgg |
| 1321 | gattccaccg agaagatcgg ggacctcttc gtggcctcgt tttccaagtc catggtgcta |
| 1381 | gatgtgtaca gtgactacgt gaacaacttc accagtgcca tgtccatcat caagaaggcc |
| 1441 | tgcctcacca agcctgcctt cctcgagttc ctcaagcgac ggcaggtgtg cagcccagac |
| 1501 | cgtgtcaccc tctacgggct gatggtcaag cccatccaga ggttcccaca gttcatactc |
| 1561 | ctgcttcagg acatgctgaa gaacaccccc aggggccatc cggacaggct gtcgctgcag |
| 1621 | ctggccctca cagagctgga gacgctggct gagaagctga acgagcagaa gcggctggct |
| 1681 | gaccaggtgg ctgagatcca gcagctgacc aagagcgtca gtgaccgcag cagcctcaac |
| 1741 | aagctgttga cctcaggcca gcggcagctg ctcctgtgtg agacgttgac ggagaccgtg |
| 1801 | tacggtgacc gcgggcagct aattaagtcc aaggagcgtc gggtcttcct gctcaacgac |
| 1861 | atgcttgtct gtgccaacat caacttcaag cctgccaacc acaggggcca gctggagatc |
| 1921 | agcagcctgg tgcccctggg gcccaagtat gtggtgaagt ggaacacggc gctgccccag |
| 1981 | gtgcaggtgg tggaggtggg ccaggacggt ggcacctatg acaaggacaa tgtgctcatc |
| 2041 | cagcactcag gcgccaagaa ggcctctgcc tcagggcagg ctcagaataa ggtgtacctc |
| 2101 | ggccccccac gcctcttcca ggagctgcag gacctgcaga aggacctggc cgtggtggag |
| 2161 | cagatcacgc ttctcatcag cacgctgcac ggcacctacc agaacctgaa catgactgtg |
| 2221 | gctcaagact ggtgcctggc cctgcagagg ctgatgcggg tgaaggagga agagatccac |
| 2281 | tcggccaaca agtgccgtct caggctcctg cttcctggga aacccgacaa gtccggccgc |
| 2341 | cccattagct tcatggtggt tttcatcacc cccaaccccc tgagcaagat ttcctgggtc |
| 2401 | aacaggttac atttggccaa aatcggactc cgggaggaga accagccagg ctggctatgc |
| 2461 | ccggatgagg acaagaagag caaagcccca ttctggtgcc cgatcctggc ctgctgcatc |
| 2521 | cctgccttct cctcccgggc actcagcctg cagcttgggg ccctggtcca cagtcctgtc |
| 2581 | aactgtcccc tgctgggttt ctcagcagtc agcacctccc ttccacaggg ctacctctgg |
| 2641 | gtcgggggcg gacaggaagg cgcagggggc caggtggaaa tcttttcctt gaaccggccc |
| 2701 | tcgccccgca ccgtcaagtc cttcccactg gcagcccctg tgctctgcat ggagtatatc |
| 2761 | ccggagctgg aggaggaggc ggagagcaga gacgagagcc cgacagttgc tgacccctcg |
| 2821 | gccacggtgc atccaaccat ctgcctcggg ctccaggatg gcagcatcct cctctacagc |
| 2881 | agtgtggaca ctggcaccca gtgcctggtg agctgcagga gcccaggtct gcagcctgtg |
| 2941 | ctctgcctgc gacacagccc cttccacctg ctcgctggcc tgcaggatgg gacccttgct |
| 3001 | gcttaccctc ggaccagcgg aggtgtcctg tgggacctgg agagccctcc cgtgtgcctg |
| 3061 | actgtggggc ccgggcctgt ccgcaccctg ttgagcctgg aggatgccgt gtgggccagc |
| 3121 | tgtgggcccc gggtcactgt cctggaagcc accaccctgc agcctcagca aagcttcgag |
| 3181 | gcgcaccagg acgaggcagt gagcgtgaca cacatggtga aggcgggcag cggcgtctgg |
| 3241 | atggccttct cctccggcac ctccatccgc ctcttccaca ctgagaccct ggagcatctg |
| 3301 | caagagatca acatcgccac caggaccacc ttcctcctgc caggccagaa gcacttgtgt |
| 3361 | gtcaccagcc tcctgatctg ccagggtctg ctctgggtgg gcactgacca gggtgtcatc |
| 3421 | gtcctgctgc ccgtgcctcg gctggaaggc atccccaaga tcacagggaa aggcatggtc |
| 3481 | tcactcaacg ggcactgtgg gcctgtggcc ttcctggctg tggctaccag catcctggcc |
| 3541 | cctgacatcc tgcggagtga ccaggaggag gctgaggggc cccgggctga ggaggacaag |
| 3601 | ccagacgggc aggcacacga gcccatgccc gatagccacg tgggccgaga gctgacccgc |
| 3661 | aagaagggca tcctcttgca gtaccgcctg cgctccaccg cacacctccc gggcccgctg |
| 3721 | ctctccatgc gggagccggc gcctgctgat ggcgcagctt tggagcacag cgaggaggac |
| 3781 | ggctccattt acgagatggc cgacgacccc gacatctggg tgcgcagccg gccctgcgcc |
| 3841 | cgcgacgccc accgcaagga gatttgctct gtggccatca tctccggcgg gcagggctac |
| 3901 | cgcaactttg gcagcgctct gggcagcagt gggaggcagg ccccgtgtgg ggagacggac |
| 3961 | agcaccctcc tcatctggca ggtgcccttg atgctatagc gcctcccctc tcccctcaga |
| 4021 | gggcacagct gcaggcctga ccaaggccac gcccggctct cgtgctctag gacctgcacg |
| 4081 | ggacttgtgg atgggcctgg actctccaga aactacttgg gcagagcaaa ggaaaacctc |
| 4141 | ttgttttaaa aaaatttttt tcagagtgtt ttggggagga gttttagggc ttggggagag |
| 4201 | ggaggacaca tctggaggaa atggccttct ttttaaaagc aaaaaacaca aaacctcaca |
| 4261 | actgcctggc aagcccagta tcacttgttt gggccctagc gggactccaa ggcagccaca |
| 4321 | cgcccctcct ggaagggtgt gtgcgtgtga gtgtgtgcga gtgtgtgggc tggtgtgtga |
| 4381 | atatctataa ataagtatat atggtgtata ttatatgtgt ataaataaag tctgtacata |
| 4441 | ttggagctct gggagatgct ggaataaaag acaagagtta catctggact tggaaaaaaa |
| 4501 | aa |

ADGRA2(GPR124) (SEQ ID NO: 44) - Homo sapiens Adhesion G protein-
coupled receptor A2, mRNA - NM_032777

| | |
|---|---|
| 1 | atccatggca cggagcggcg gcggcggcgg cagcaggagc ccggcgcgat ccgctaggtc |
| 61 | ccagcccagc gcccagcgag caggcgacgc ggaggggccg ggcctccagt gtcccgaggg |
| 121 | ccgggcgctg agactccggc cgcgcagctg ggagctgccc gcgctgcgct gacagccgcg |
| 181 | ccgacgtcct ccccgccggg gcgctcgcag gacatgcccc cggggcgcgg cggcggggac |
| 241 | cccggggctc gcctccgccc agggcccccc tccacgccct cgggagcccc gggcccccgc |
| 301 | tgagcactcc tcccgcacgc ctgggtccct ccggccggcg cgcagcccgg ccccagcgct |
| 361 | gtgggtcccc gcggggcgat gggttgatgg gcgccggggg acgcaggatg cggggggcgc |
| 421 | ccgcgcgcct gctgctgccg ctgctgccgt ggctcctgct gctcctggcg cccgaggctc |
| 481 | ggggcgcgcc cggctgcccg ctatccatcc gcagctgcaa gtgctcgggg gagcggccca |
| 541 | aggggctgag cggcggcgtc cctggcccgg ctcggcggag ggtggtgtgc agcggcgggg |
| 601 | acctcccgga gcctcccgag cccggccttc tgcctaacgg caccgttacc ctgctcttga |
| 661 | gcaataacaa gatcacgggg ctccgcaatg gctccttcct gggactgtca ctgctggaga |
| 721 | agctggacct gaggaacaac atcatcagca cagtgcagcc gggcgccttc ctgggcctgg |
| 781 | gggagctgaa gcgtttagat ctctccaaca accggattgg ctgtctcacc tccgagacct |
| 841 | tccagggcct ccccaggctt ctccgactaa acatatctgg aaacatcttc tccagtctgc |
| 901 | aacctggggt ctttgatgag ctgccagccc ttaaggttgt ggacttgggc accgagttcc |
| 961 | tgacctgtga ctgccacctg cgctggctgc tgccctgggc ccagaatcgc tccctgcagc |
| 1021 | tgtcggaaca cacgctctgt gcttacccca gtgccctgca tgctcaggcc ctgggcagcc |
| 1081 | tccaggaggc ccagctctgc tgcgaggggg ccctggagct gcacacacac cacctcatcc |
| 1141 | cgtccctacg ccaagtggtg ttccaggggg atcggctgcc cttccagtgc tctgccagct |
| 1201 | acctgggcaa cgacacccgc atccgctggt accacaaccg agcccctgtg gagggtgatg |
| 1261 | agcaggcggg catcctcctg gccgagagcc tcatccacga ctgcaccttc atcaccagtg |
| 1321 | agctgacgct gtctcacatc ggcgtgtggg cctcaggcga gtgggagtgc accgtgtcca |
| 1381 | tggcccaagg caacgccagc aagaaggtgg agatcgtggt gctggagacc tctgcctcct |
| 1441 | actgccccgc cgagcgtgtt gccaacaacc gcggggactt caggtggccc cgaactctgg |
| 1501 | ctggcatcac agcctaccag tcctgcctgc agtatccctt cacctcagtg cccctgggcg |
| 1561 | ggggtgcccc gggcacccga gcctcccgcc ggtgtgaccg tgccggccgc tgggagccag |
| 1621 | gggactactc ccactgtctc tacaccaacg acatcaccag ggtgctgtac accttcgtgc |
| 1681 | tgatgcccat caatgcctcc aatgcgctga ccctggctca ccagctgcgc gtgtacacag |
| 1741 | ccgaggccgc tagcttttca gacatgatgg atgtagtcta tgtggctcag atgatccaga |
| 1801 | aatttttggg ttatgtcgac cagatcaaag agctggtaga ggtgatggtg gacatggcca |
| 1861 | gcaacctgat gctggtggac gagcacctgc tgtggctggc ccagcgcgag gacaaggcct |
| 1921 | gcagccgcat cgtgggtgcc ctggagcgca ttgggggggc cgccctcagc ccccatgccc |
| 1981 | agcacatctc agtgaatgcg aggaacgtgg cattggaggc ctacctcatc aagccgcaca |
| 2041 | gctacgtggg cctgacctgc acagccttcc agaggaggga gggaggggtg ccgggcacac |
| 2101 | ggccaggaag ccctggccag aaccccccac ctgagcccga gcccccagct gaccagcagc |
| 2161 | tccgcttccg ctgcaccacc gggaggccca atgtttctct gtcgtccttc cacatcaaga |
| 2221 | acagcgtggc cctggcctcc atccagctgc ccccgagtct attctcatcc cttccggctg |
| 2281 | ccctggctcc cccggtgccc ccagactgca ccctgcaact gctcgtcttc cgaaatggcc |
| 2341 | gcctcttcca cagccacagc aacacctccc gccctggagc tgctgggcct ggcaagaggc |
| 2401 | gtggcgtggc cacccccgtc atcttcgcag gaaccagtgg ctgtggcgtg ggaaacctga |
| 2461 | cagagccagt ggccgtttcg ctgcggcact gggctgaggg agccgaacct gtggccgctt |
| 2521 | ggtggagcca ggaggggccc ggggaggctg ggggctggac ctcggagggc tgccagctcc |
| 2581 | gctccagcca gcccaatgtc agcgccctgc actgccagca cttgggcaat gtggccgtgc |
| 2641 | tcatggagct gagcgccttt cccagggagg tggggggcgc cggggcaggg ctgcaccccg |
| 2701 | tggtataccc ctgcacggcc ttgctgctgc tctgcctctt cgccaccatc atcacctaca |
| 2761 | tcctcaacca cagctccatc cgtgtgtccc ggaaaggctg gcacatgctg ctgaacttgt |
| 2821 | gcttccacat agccatgacc tctgctgtct ttgcgggggg catcacactc accaactacc |
| 2881 | agatggtctg ccaggcggtg ggcatcaccc tgcactactc ctccctatcc acgctgctct |
| 2941 | ggatgggcgt gaaggcgcga gtgctccata aggagctcac ctggagggca ccccctccgc |
| 3001 | aagaagggga ccccgctctg cctactccca gtcctatgct ccggttctat ttgatcgctg |
| 3061 | gagggattcc actcattatc tgtggcatca cagctgcagt caacatccac aactaccggg |
| 3121 | accacagccc ctactgctgg ctggtgtggc gtccaagcct tggcgccttc tacatccctg |
| 3181 | tggctttgat tctgctcatc acctggatct atttcctgtg cgccgggcta cgcttacggg |
| 3241 | gtcctctggc acagaacccc aaggcgggca acagcagggc ctccctggag gcaggggagg |
| 3301 | agctgagggg ttccaccagg ctcaggggca gcggccccct cctgagtgac tcaggttccc |
| 3361 | ttcttgctac tgggagcgcg cgagtgggga cgcccgggcc cccggaggat ggtgacagcc |
| 3421 | tctattctcc gggagtccag ctaggggcgc tggtgaccac gcacttcctg tacttggcca |
| 3481 | tgtgggcctg cggggctctg gcagtgtccc agcgctggct gccccgggtg gtgtgcagct |
| 3541 | gcttgtacgg ggtggcagcc tccgccctgg gcctcttcgt cttcactcac cactgtgcca |
| 3601 | ggcggaggga cgtgagagcc tcgtggcgcg cctgctgccc ccctgcctct cccgcggccc |
| 3661 | cccatgcccc gccccgggcc ctgcccgccg ccgcagagga cggttccccg gtgttcgggg |
| 3721 | agggcccccc ctccctcaag tcctccccaa gcggcagcag cggccatccg ctggctctgg |
| 3781 | gcccctgcaa gctcaccaac ctgcagctgg cccagagtca ggtgtgcgag gcgggggcgg |
| 3841 | cggccggcgg ggaaggagag ccggagccgg cgggcacccg gggaaacctc gcccaccgcc |
| 3901 | accccaacaa cgtgcaccac gggcgtcggg cgcacaagag ccgggccaag ggacaccgcg |
| 3961 | cgggggaggc ctgcggcaag aaccggctca aggccctgcg cgggggcgcg gcgggggcgc |
| 4021 | tggagctgct gtccagcgag agcggcagtc tgcacaacag ccccaccgac agctacctgg |
| 4081 | gcagcagccg caacagcccg ggcgccggcc tgcagctgga aggcgagccc atgctcacgc |
| 4141 | cgtccgaggg cagcgacacc agcgccgcgc cgctttctga ggcgggccgg gcaggccagc |
| 4201 | gccgcagcgc cagccgcgac agtctcaagg gcggcggcgc gctggagaag gagagccatc |
| 4261 | gccgctcgta cccgctcaac gccgccagcc taaacggcgc ccccaagggg ggcaagtacg |
| 4321 | acgacgtcac cctgatgggc gcggaggtag ccagcggcgg ctgcatgaag accggactct |
| 4381 | ggaagagcga aactaccgtc taaggtgggg cgggcgacgc ggtagacggg ctggccacgc |
| 4441 | ggctcgttcc cccgctcctc ggggccctcc aaggtgtctc cgtagtcagc aggttggagg |
| 4501 | cagaggagcc gatggctgga ggaagcccac aggcggatgt tccccacttg cctagagggc |
| 4561 | atccctctgg ggtagcgaca gacaatccca gaaacacgca taatacattt ccgtccagcc |
| 4621 | cggggcagtc tgactgtcgg tgccctccca ggaacgggga aggcctccgt ctgtgtgaaa |
| 4681 | gggcacagca catcccaggt gcaccctccc caagtactcc caccccgcct actgtccatg |
| 4741 | cggcctcact gggggccatc agcctcacca gcaaagcaga gatgagagcg tgggaactgt |
| 4801 | gttctttcct ccctgccctc tactgatttc agcccagccc ctgcctagat cctaggtccc |
| 4861 | ttttcctccc gagtttggct ggcacgagag ctagcccagc acatgaagca ggtgatgtta |
| 4921 | agtcacaagg tgctgctttt cagatccact atgcaagagg ggagggtggg gccacgtgaa |
| 4981 | aggcagctct agacatcaac cagtcctggg ggaggggagt gggaaccggg cacaactagg |
| 5041 | aacaatgcca ccattcccac aggagtggta cttaaaccag acagcagggt tcagaggtgg |
| 5101 | cacaccggga caaagctgag gccctgcacc tcaacagctg actgccaggt gcctgtgggt |
| 5161 | gaactgaggg gagtagaggg agagggcagg tggaactggg gcagaatcta gtcatgccct |
| 5221 | aaagctagtc ctgtaaacaa tggtgcccca gaaagctgca ggtggtgttt ggagaagcag |
| 5281 | ttacttttca gttacaagac ccatctccct agtctcagcc ttacaacacc acgggactaa |
| 5341 | ggaagagcac ttccttgcct ccgtaaggcc agaggaagaa ccatcccaat catttgatct |
| 5401 | ccagctccac agtagagaga aacctacaaa atgtcaaacc agcttcccga ctcccaggag |
| 5461 | ctcaagccaa gcccagaggc agtggctggg gtccctgcag gtcatgaggg gcctatgcct |
| 5521 | ttactccttt taaacaccag cacccgtctt ttccccaacc taaaaccaac caccagcatt |
| 5581 | tcactacagg accaaatgga aaccgaggga accctgggtc ttgggaagaa caacaggaaa |
| 5641 | ccaaggtctg acctagggtt ccctcccagt cttcacatca ctctggcctc atcaccaagg |
| 5701 | tgacagagga cacaggggag ggggaaaacc cacacacact ccttggaatg ggtcctgtta |
| 5761 | tttatgcttg ctgcacagac atattagaag aaaaaaaaaa gctttgtatt attcttccac |
| 5821 | atatgctggc tgctgtttac acaccctgcc aatgccttag cactggagag ctttttgcaa |
| 5881 | tatgctgggg aaaggggagg gagggaatga aagtgccaaa gaaaacatgt ttttaagaac |
| 5941 | tcgggtttta tacaatagaa tgttttctag cagatgcctc ttgttttaat atattaaaat |
| 6001 | tttgcaaagc cctttgagct actgccttag tctaaaaaaa aaaaaaaaaa |

IL1RN (SEQ ID NO: 45) interleukin-1 receptor antagonist protein
isoform 4 NM_173843.2

| | |
|---|---|
| 1 | gggcagctcc accctgggag ggactgtggc ccaggtactg cccgggtgct actttatggg |
| 61 | cagcagctca gttgagttag agtctggaag acctcagaag acctcctgtc ctatgaggcc |
| 121 | ctccccatgg ctttaggggg attataaaac taatcatcaa agccaagaag gcaagagcaa |
| 181 | gcatgtaccg ctgaaaacac aagataactg cataagtaat gactttcagt gcagattcat |
| 241 | agctaaccca taaactgctg gggcaaaaat catcttggaa ggctctgaac ctcagaaagg |
| 301 | attcacaaga cgatctgccg accctctggg agaaaatcca gcaagatgca agccttcaga |
| 361 | atctgggatg ttaaccagaa gaccttctat ctgaggaaca accaactagt tgctggatac |
| 421 | ttgcaaggac caaatgtcaa tttagaagaa aagatagatg tggtacccat tgagcctcat |
| 481 | gctctgttct tgggaatcca tggagggaag atgtgcctgt cctgtgtcaa gtctggtgat |
| 541 | gagaccagac tccagctgga ggcagttaac atcactgacc tgagcgagaa cagaaagcag |
| 601 | gacaagcgct tcgccttcat ccgctcagac agtggcccca ccaccagttt tgagtctgcc |
| 661 | gcctgccccg gttggttcct ctgcacagcg atggaagctg accagcccgt cagcctcacc |
| 721 | aatatgcctg acgaaggcgt catggtcacc aaattctact tccaggagga cgagtagtac |
| 781 | tgcccaggcc tgcctgttcc cattcttgca tggcaaggac tgcagggact gccagtcccc |
| 841 | ctgccccagg gctcccggct atgggggcac tgaggaccag ccattgaggg gtggaccctc |
| 901 | agaaggcgtc acaacaacct ggtcacagga ctctgcctcc tcttcaactg accagcctcc |
| 961 | atgctgcctc cagaatggtc tttctaatgt gtgaatcaga gcacagcagc ccctgcacaa |
| 1021 | agcccttcca tgtcgcctct gcattcagga tcaaaccccg accacctgcc caacctgctc |
| 1081 | tcctcttgcc actgcctctt cctccctcat tccaccttcc catgccctgg atccatcagg |
| 1141 | ccacttgatg acccccaacc aagtggctcc cacaccctgt tttacaaaaa agaaaagacc |
| 1201 | agtccatgag ggaggttttt aagggtttgt ggaaaatgaa aattaggatt tcatgatttt |
| 1261 | tttttttcag tccccgtgaa ggagagccct tcatttggag attatgttct ttcggggaga |
| 1321 | ggctgaggac ttaaaatatt cctgcatttg tgaaatgatg gtgaaagtaa gtggtagctt |
| 1381 | ttcccttctt tttcttcttt ttttgtgatg tcccaacttg taaaaattaa aagttatggt |
| 1441 | actatgttag ccccataatt ttttttttcc ttttaaaaca cttccataat ctggactcct |
| 1501 | ctgtccaggc actgctgccc agcctccaag ctccatctcc actccagatt ttttacagct |
| 1561 | gcctgcagta ctttacctcc tatcagaagt ttctcagctc ccaaggctct gagcaaatgt |
| 1621 | ggctcctggg ggttctttct tcctctgctg aaggaataaa ttgctccttg acattgtaga |
| 1681 | gcttctggca cttggagact tgtatgaaag atggctgtgc ctctgcctgt ctcccccacc |
| 1741 | gggctgggag ctctgcagag caggaaacat gactcgtata tgtctcaggt ccctgcaggg |
| 1801 | ccaagcacct agcctcgctc ttggcaggta ctcagcgaat gaatgctgta tatgttgggt |
| 1861 | gcaaagttcc ctacttcctg tgacttcagc tctgttttac aataaaatct tgaaaatgcc |
| 1921 | taaaaaaaaa aaaaaaaaaa aaaaaaaaaa aaaaaaaaaa aaaaaaaaaa aaa |

NLRP3 (SEQ ID NO: 46) NACHT, LRR and PYD domains-containing protein
3 isoform a, transcript variant 1 NM_004895.4

| | |
|---|---|
| 1 | gtagatgagg aaactgaagt tgaggaatag tgaagagttt gtccaatgtc atagccccgt |
| 61 | aatcaacggg acaaaaattt tcttgctgat gggtcaagat ggcatcgtga agtggttgtt |
| 121 | caccgtaaac tgtaatacaa tcctgtttat ggatttgttt gcatattttt ccctccatag |
| 181 | ggaaaccttt cttccatggc tcaggacaca ctcctggatc gagccaacag gagaactttc |
| 241 | tggtaagcat ttggctaact tttttttttt tgagatggag tcttgctgtg tcgcctaggc |
| 301 | tggagtgcag tggcgtgatc ttggctcact gcagcctcca cttcccgggt tcaatcaatt |
| 361 | ctcctacctc aacttcctga gtagctggga ttacaggcgc ccgccaccac acccggctca |
| 421 | tttttgtact tttagtagag acacagtttt gccatgttgg ccaggctggt cttgaattcc |
| 481 | tcagctcagg tgatctgcct gccttggcct ctcaaagtgc tgggattaca ggcgtgagcc |
| 541 | actgtgcccg gccttggcta acttttcaaa attaaagatt ttgacttgtt acagtcatgt |
| 601 | gacatttttt tctttctgtt tgctgagttt ttgataattt atatctctca aagtggagac |
| 661 | tttaaaaaag actcatccgt gtgccgtgtt cactgcctgg tatcttagtg tggaccgaag |
| 721 | cctaaggacc ctgaaaacag ctgcagatga agatggcaag cacccgctgc aagctggcca |
| 781 | ggtacctgga ggacctggag gatgtggact tgaagaaatt taagatgcac ttagaggact |
| 841 | atcctcccca gaagggctgc atccccctcc cgaggggtca gacagagaag gcagaccatg |
| 901 | tggatctagc cacgctaatg atcgacttca atggggagga gaaggcgtgg gccatggccg |
| 961 | tgtggatctt cgctgcgatc aacaggagag acctttatga gaaagcaaaa agagatgagc |
| 1021 | cgaagtgggg ttcagataat gcacgtgttt cgaatcccac tgtgatatgc caggaagaca |
| 1081 | gcattgaaga ggagtggatg ggtttactgg agtacctttc gagaatctct atttgtaaaa |
| 1141 | tgaagaaaga ttaccgtaag aagtacagaa agtacgtgag aagcagattc cagtgcattg |
| 1201 | aagacaggaa tgcccgtctg ggtgagagtg tgagcctcaa caaacgctac acacgactgc |
| 1261 | gtctcatcaa ggagcaccgg agccagcagg agagggagca ggagcttctg gccatcggca |
| 1321 | agaccaagac gtgtgagagc cccgtgagtc ccattaagat ggagttgctg tttgaccccg |
| 1381 | atgatgagca ttctgagcct gtgcacaccg tggtgttcca gggggcggca gggattggga |
| 1441 | aaacaatcct ggccaggaag atgatgttgg actgggcgtc ggggacactc taccaagaca |
| 1501 | ggtttgacta tctgttctat atccactgtc gggaggtgag ccttgtgaca cagaggagcc |
| 1561 | tgggggacct gatcatgagc tgctgccccg acccaaaccc acccatccac aagatcgtga |
| 1621 | gaaaaccctc cagaatcctc ttcctcatgg acggcttcga tgagctgcaa ggtgcctttg |
| 1681 | acgagcacat aggaccgctc tgcactgact ggcagaaggc cgagcgggga gacattctcc |
| 1741 | tgagcagcct catcagaaag aagctgcttc ccgaggcctc tctgctcatc accacgagac |
| 1801 | ctgtggccct ggagaaactg cagcacttgc tggaccatcc tcggcatgtg gagatcctgg |
| 1861 | gtttctccga ggccaaaagg aaagagtact tcttcaagta cttctctgat gaggcccaag |
| 1921 | ccagggcagc cttcagtctg attcaggaga acgaggtcct cttcaccatg tgcttcatcc |
| 1981 | ccctggtctg ctggatcgtg tgcactggac tgaaacagca gatggagagt ggcaagagcc |
| 2041 | ttgcccagac atccaagacc accaccgcgg tgtacgtctt cttcctttcc agtttgctgc |
| 2101 | agccccgggg agggagccag gagcacggcc tctgcgccca cctctggggg ctctgctctt |
| 2161 | tggctgcaga tggaatctgg aaccagaaaa tcctgtttga ggagtccgac ctcaggaatc |
| 2221 | atggactgca gaaggcggat gtgtctgctt tcctgaggat gaacctgttc caaaaggaag |
| 2281 | tggactgcga gaagttctac agcttcatcc acatgacttt ccaggagttc tttgccgcca |
| 2341 | tgtactacct gctggaagag gaaaaggaag gaaggacgaa cgttccaggg agtcgtttga |
| 2401 | agcttcccag ccgagacgtg acagtccttc tggaaaacta tggcaaattc gaaaaggggt |
| 2461 | atttgatttt tgttgtacgt ttcctctttg gcctggtaaa ccaggagagg acctcctact |
| 2521 | tggagaagaa attaagttgc aagatctctc agcaaatcag gctggagctg ctgaaatgga |
| 2581 | ttgaagtgaa agccaaagct aaaaagctgc agatccagcc cagccagctg gaattgttct |
| 2641 | actgtttgta cgagatgcag gaggaggact tcgtgcaaag ggccatggac tatttcccca |
| 2701 | agattgagat caatctctcc accagaatgg accacatggt ttcttccttt tgcattgaga |
| 2761 | actgtcatcg ggtggagtca ctgtccctgg ggtttctcca taacatgccc aaggaggaag |
| 2821 | aggaggagga aaaggaaggc cgacaccttg atatggtgca gtgtgtcctc ccaagctcct |
| 2881 | ctcatgctgc ctgttctcat ggattggtga acagccacct cacttccagt ttttgccggg |
| 2941 | gcctcttttc agttctgagc accagccaga gtctaactga attggacctc agtgacaatt |
| 3001 | ctctggggga cccagggatg agagtgttgt gtgaaacgct ccagcatcct ggctgtaaca |
| 3061 | ttcggagatt gtggttgggg cgctgtggcc tctcgcatga gtgctgcttc gacatctcct |
| 3121 | tggtcctcag cagcaaccag aagctggtgg agctggacct gagtgacaac gccctcggtg |
| 3181 | acttcggaat cagacttctg tgtgtgggac tgaagcacct gttgtgcaat ctgaagaagc |
| 3241 | tctggttggt cagctgctgc ctcacatcag catgttgtca ggatcttgca tcagtattga |
| 3301 | gcaccagcca ttccctgacc agactctatg tgggggagaa tgccttggga gactcaggag |
| 3361 | tcgcaatttt atgtgaaaaa gccaagaatc cacagtgtaa cctgcagaaa ctggggttgg |
| 3421 | tgaattctgg ccttacgtca gtctgttgtt cagctttgtc ctcggtactc agcactaatc |
| 3481 | agaatctcac gcacctttac ctgcgaggca acactctcgg agacaagggg atcaaactac |
| 3541 | tctgtgaggg actcttgcac cccgactgca agcttcaggt gttggaatta gacaactgca |
| 3601 | acctcacgtc acactgctgc tgggatcttt ccacacttct gacctccagc cagagcctgc |
| 3661 | gaaagctgag cctgggcaac aatgacctgg gcgacctggg ggtcatgatg ttctgtgaag |
| 3721 | tgctgaaaca gcagagctgc ctcctgcaga acctggggtt gtctgaaatg tatttcaatt |
| 3781 | atgagacaaa aagtgcgtta gaaacacttc aagaagaaaa gcctgagctg accgtcgtct |
| 3841 | ttgagccttc ttggtaggag tggaaacggg gctgccagac gccagtgttc tccggtccct |
| 3901 | ccagctgggg gccctcaggt ggagagagct gcgatccatc caggccaaga ccacagctct |
| 3961 | gtgatccttc cggtggagtg tcggagaaga gagcttgccg acgatgcctt cctgtgcaga |
| 4021 | gcttgggcat ctcctttacg ccagggtgag gaagacacca ggacaatgac agcatcgggt |
| 4081 | gttgttgtca tcacagcgcc tcagttagag gatgttcctc ttggtgacct catgtaatta |
| 4141 | gctcattcaa taaagcactt tctttatttt tctcttctct gtctaacttt ctttttccta |
| 4201 | tcttttttct tctttgttct gtttactttt gctcatatca tcattcccgc tatctttcta |
| 4261 | ttaactgacc ataacacaga actagttgac tatatattat gttgaaattt tatggcagct |
| 4321 | atttatttat ttaaattttt tgtaacagtt ttgttttcta ataagaaaaa tccatgcttt |
| 4381 | ttgtagctgg ttgaaaattc aggaatatgt aaaacttttt ggtatttaat taaattgatt |
| 4441 | ccttttctta attttaaaaa aaaaaaaaaa |

RBP4 (SEQ ID NO: 47) retinol binding protein 4 NM_001322517.1

| | |
|---|---|
| 1 | ggcggccgct ggcacgagtg cagggtaact gagccagggc cgctggcgca tttggcctgg |
| 61 | ccgaggccac cccgcgcggc cgctccactg tgcccgaggc tgtcctggag gtgaggccgg |
| 121 | cccacaggga ccctgcccgt gcccgggctc cgggcggatt cctgggcaag atgaagtggg |
| 181 | tgtgggcgct cttgctgttg gcggcgctgg gcagcggccg cgcggagcgc gactgccgag |
| 241 | tgagcagctt ccgagtcaag gagaacttcg acaaggctcg cttctctggg acctggtacg |
| 301 | ccatggccaa gaaggacccc gagggcctct ttctgcagga caacatcgtc gcggagttct |
| 361 | ccgtggacga gaccggccag atgagcgcca cagccaaggg ccgagtccgt cttttgaata |
| 421 | actgggacgt gtgcgcagac atggtgggca ccttcacaga caccgaggac cctgccaagt |
| 481 | tcaagatgaa gtactggggc gtagcctcct ttctccagaa aggaaatgat gaccactgga |
| 541 | tcgtcgacac agactacgac acgtatgccg tgcagtactc ctgccgcctc ctgaacctcg |
| 601 | atggcacctg tgctgacagc tactccttcg tgttttcccg ggaccccaac ggcctgcccc |
| 661 | cagaagcgca gaagattgta aggcagcggc aggaggagct gtgcctggcc aggcagtaca |
| 721 | ggctgatcgt ccacaacggt tactgcgatg gcagatcaga aagaaacctt ttgtagcaat |
| 781 | atcaagaatc tagtttcatc tgagaacttc tgattagctc tcagtcttca gctctattta |
| 841 | tcttaggagt ttaatttgcc cttctctccc catcttccct cagttcccat aaaaccttca |
| 901 | ttacacataa agatacacgt gggggtcagt gaatctgctt gcctttcctg aaagtttctg |
| 961 | gggcttaaga ttccagactc tgattcatta aactatagtc acccgtgtcc tgtga |

MPP3 (SEQ ID NO: 48) membrane palmitoylated protein 3, transcript
variant 1, mRNA NM_001932.4

| | |
|---|---|
| 1 | aaggcggagc cagcggcggc ccaggctctg ggcactcgcg cggctcccgt tcgcggagca |
| 61 | cacatcccgg cgcgacgagg ttccttcgga gagcgagcgg gagtcggtgc tcggcctcct |
| 121 | gcggggagca gcccgaggaa tctgcaggga gaggtcggga ggtgacaacg ccagcatgcc |
| 181 | agtgctatcg gaggactctg gtttgcatga aaccctggcc ctgctgacct cccagctcag |
| 241 | acctgactcc aaccacaagg aggagatggg cttcctgagg gatgttttca gtgaaaaaag |
| 301 | cctcagttac ttaatgaaga ttcatgagaa gcttcgctat tatgaaaggc aaagtccaac |
| 361 | cccagttctg cacagcgctg tggccctcgc tgaggacgtg atggaggagt tgcaggccgc |
| 421 | ctccgtgcac agtgatgaga gggagctgct ccagctgctg tccaccccgc acctgagggc |
| 481 | tgtgctcatg gtacatgaca cggttgccca gaagaatttt gaccccgttc tcccgcctct |
| 541 | gcctgacaat atcgatgagg attttgatga ggaatcggtg aagatcgtcc gcttggtgaa |
| 601 | gaacaaggaa cccctgggtg ccaccatccg gcgggacgag cactcagggg ctgttgtggt |
| 661 | ggccaggatc atgcgaggag gcgcagcaga caggagcggc ctggtccacg ttggagatga |
| 721 | gctccgagaa gtgaacggga tcgcagtcct gcacaagcgg cccgacgaga tcagccagat |
| 781 | tctggcccag tcccagggat ccatcaccct aaaaatcatc ccagccaccc aggaggaaga |
| 841 | tcgcttaaag gagagcaagg tgttcatgcg cgccctcttc cactacaacc ctcgggagga |
| 901 | ccgggccatc ccttgccagg aggcgggcct gcccttccag cgcaggcagg tcctggaggt |
| 961 | ggtgagccag gacgacccca cgtggtggca ggccaagcga gtcggggaca ccaaccttcg |
| 1021 | agccggcctc atcccctcca aggggttcca ggagagacga ctaagctacc ggagagccgc |
| 1081 | gggcaccctg ccgagccccc agagcctcag gaagcccccc tatgatcagc cttgtgacaa |
| 1141 | agagacctgt gactgtgagg gctacctcaa agggcactat gtggctggtc ttcggaggag |
| 1201 | cttccggctg ggctgtaggg agagactggg tggctcgcag gaaggaaaga tgtcctccgg |
| 1261 | agctgagtct ccggagctgc tgacttacga agaggtggcc aggtaccaac accagcccgg |
| 1321 | agagcggccc cgcctggtgg ttctgatcgg gtctctggga gcccgactgc acgagctgaa |
| 1381 | gcaaaaggtg gtggctgaga acccacagca ctttggcgtc gctgttccac ataccaccag |
| 1441 | gccccgaaag agccatgaga aggaaggagt ggaatatcac tttgtgtcta agcaagcatt |
| 1501 | tgaggccgac ttacatcaca acaagttcct ggaacatggt gaatataagg aaaatctgta |
| 1561 | tggaaccagc ctggaggcca ttcaggctgt tatggccaaa aacaaagttt gtttggtgga |
| 1621 | tgtggagcca gaagcactga aacaactgag gacctcagaa tttaaaccct atattatatt |
| 1681 | tgtaaagcct gcaattcagg aaaaaagaaa aacgccacct atgtccccag cttgtgagga |
| 1741 | cacagcagcc ccatttgatg agcagcagca agagatggcc gcttctgccg ccttcataga |
| 1801 | ccggcattac gggcacctgg tagacgccgt gctggtgaag gaggatctcc agggtgccta |
| 1861 | cagccagctc aaagtggtct tagagaagct gagcaaggac actcactggg tacctgttag |
| 1921 | ttgggtcagg taactttatc ccagaacatc caagctggac gggaccttga agatcatcta |
| 1981 | gtccagactc cctcatttta ccatcaagga atctcaagcg cagagaggga gagaattctc |
| 2041 | cacaaattcc atcatcgaga agagtataag tgggaagtct tgtttgttgt tggtttttgt |
| 2101 | ctgttgtttt tcactgcacc tctttggatc atgatttgaa aggggcatat cagaaaacaa |
| 2161 | cacatttcat ttattaaagt atcacaggca agctgaccct gattctttgt accaaagtta |
| 2221 | agtagccact gtcttttgtg ggtggtagtg gttaatttat acagtactga ttcgcagaat |
| 2281 | gtttaagctt tttaaacata gtgacgctta gtagtttttt tggaagctaa cttgttttat |
| 2341 | ccaaggggat tttacatgta actgaagttc ccctgtcttc aagcactaaa acgttgatct |
| 2401 | taaccttttt tttgaagtgc ttgcctggta atagaaaacg ggttctctgc ctattttaaa |
| 2461 | atagtgaata tacgtaaatt ttctctggaa ggctgaggca cacttcacca tcaacatgaa |
| 2521 | ttactgtact atcctgtact gcagtggtgc cttcagggac tcgaggaatg taaggttgcc |
| 2581 | tttccccttt ctaaataccc tcagattcct aacatcgagc ccatgctttg tttgatttgt |
| 2641 | tctattccat ccattgtccc ttttgttact gacagttgcc ttggtcctag ccagtccctg |
| 2701 | ccatgagatc ataggggttc ccattgtgct agatcttggg aaaccagatg actctccctg |
| 2761 | tcaaaactat ggctacgtca ctgtaaacca tttctgtcaa gaataaaagt atgtagaccc |
| 2821 | agagtgtggg cctaaaaaaa aaaaaaaaaa a |

KIF2C (SEQ ID NO: 49) kinesin-like protein KIF2C isoform 1
NM_006845.3

| | |
|---|---|
| 1 | acgcttgcgc gcgggattta aactgcggcg gtttacgcgg cgttaagact tcgtagggtt |
| 61 | agcgaaattg aggtttcttg gtattgcgcg tttctcttcc ttgctgactc tccgaatggc |
| 121 | catggactcg tcgcttcagg cccgcctgtt tcccggtctc gctatcaaga tccaacgcag |
| 181 | taatggttta attcacagtg ccaatgtaag gactgtgaac ttggagaaat cctgtgtttc |
| 241 | agtggaatgg gcagaaggag gtgccacaaa gggcaaagag attgattttg atgatgtggc |
| 301 | tgcaataaac ccagaactct tacagcttct tcccttacat ccgaaggaca atctgccctt |
| 361 | gcaggaaaat gtaacaatcc agaaacaaaa acggagatcc gtcaactcca aaattcctgc |
| 421 | tccaaaagaa agtcttcgaa gccgctccac tcgcatgtcc actgtctcag agcttcgcat |
| 481 | cacggctcag gagaatgaca tggaggtgga gctgcctgca gctgcaaact cccgcaagca |
| 541 | gttttcagtt cctcctgccc ccactaggcc ttcctgccct gcagtggctg aaataccatt |
| 601 | gaggatggtc agcgaggaga tggaagagca agtccattcc atccgaggca gctcttctgc |
| 661 | aaaccctgtg aactcagttc ggaggaaatc atgtcttgtg aaggaagtgg aaaaaatgaa |
| 721 | gaacaagcga gaagagaaga aggcccagaa ctctgaaatg agaatgaaga gagctcagga |
| 781 | gtatgacagt agttttccaa actgggaatt tgcccgaatg attaaagaat ttcgggctac |
| 841 | tttggaatgt catccactta ctatgactga tcctatcgaa gagcacagaa tatgtgtctg |
| 901 | tgttaggaaa cgcccactga ataagcaaga attggccaag aaagaaattg atgtgatttc |
| 961 | cattcctagc aagtgtctcc tcttggtaca tgaacccaag ttgaaagtgg acttaacaaa |
| 1021 | gtatctggag aaccaagcat tctgctttga ctttgcattt gatgaaacag cttcgaatga |
| 1081 | agttgtctac aggttcacag caaggccact ggtacagaca atctttgaag gtggaaaagc |
| 1141 | aacttgtttt gcatatggcc agacaggaag tggcaagaca catactatgg gcggagacct |
| 1201 | ctctgggaaa gcccagaatg catccaaagg gatctatgcc atggcctccc gggacgtctt |
| 1261 | cctcctgaag aatcaaccct gctaccggaa gttgggcctg gaagtctatg tgacattctt |
| 1321 | cgagatctac aatgggaagc tgtttgacct gctcaacaag aaggccaagc tgcgcgtgct |
| 1381 | ggaggacggc aagcaacagg tgcaagtggt ggggctgcag gagcatctgg ttaactctgc |
| 1441 | tgatgatgtc atcaagatga tcgacatggg cagcgcctgc agaacctctg ggcagacatt |
| 1501 | tgccaactcc aattcctccc gctcccacgc gtgcttccaa attattcttc gagctaaagg |
| 1561 | gagaatgcat ggcaagttct ctttggtaga tctggcaggg aatgagcgag gcgcggacac |
| 1621 | ttccagtgct gaccggcaga cccgcatgga gggcgcagaa atcaacaaga gtctcttagc |
| 1681 | cctgaaggag tgcatcaggg ccctgggaca gaacaaggct cacaccccgt tccgtgagag |
| 1741 | caagctgaca caggtgctga gggactcctt cattggggag aactctagga cttgcatgat |
| 1801 | tgccacgatc tcaccaggca taagctcctg tgaatatact ttaaacaccc tgagatatgc |
| 1861 | agacagggtc aaggagctga gcccccacag tgggcccagt ggagagcagt tgattcaaat |
| 1921 | ggaaacagaa gagatggaag cctgctctaa cggggcgctg attccaggca atttatccaa |
| 1981 | ggaagaggag gaactgtctt cccagatgtc cagctttaac gaagccatga ctcagatcag |
| 2041 | ggagctggag gagaaggcta tggaagagct caaggagatc atacagcaag gaccagactg |
| 2101 | gcttgagctc tctgagatga ccgagcagcc agactatgac ctggagacct ttgtgaacaa |
| 2161 | agcggaatct gctctggccc agcaagccaa gcatttctca gccctgcgag atgtcatcaa |
| 2221 | ggccttgcgc ctggccatgc agctggaaga gcaggctagc agacaaataa gcagcaagaa |
| 2281 | acggccccag tgacgactgc aaataaaaat ctgtttggtt tgacacccag cctcttccct |
| 2341 | ggccctcccc agagaacttt gggtacctgg tgggtctagg cagggtctga gctgggacag |
| 2401 | gttctggtaa atgccaagta tgggggcatc tgggcccagg gcagctgggg agggggtcag |
| 2461 | agtgacatgg gacactcctt ttctgttcct cagttgtcgc cctcacgaga ggaaggagct |
| 2521 | cttagttacc cttttgtgtt gcccttcttt ccatcaaggg gaatgttctc agcatagagc |
| 2581 | tttctccgca gcatcctgcc tgcgtggact ggctgctaat ggagagctcc ctggggttgt |
| 2641 | cctggctctg gggagagaga cggagccttt agtacagcta tctgctggct ctaaaccttc |
| 2701 | tacgcctttg ggccgagcac tgaatgtctt gtactttaaa aaaatgtttc tgagacctct |
| 2761 | ttctacttta ctgtctccct agagatccta gaggatccct actgttttct gttttatgtg |
| 2821 | tttatacatt gtatgtaaca ataaagagaa aaaataaatc agctgtttaa gtgtgtggaa |
| 2881 | aaaaaaaaaa aaaaaa |

MAP1A (SEQ ID NO: 50) - Homo sapiens microtubule associated protein
1A (MAP1A), mRNA - NM_002373

| | |
|---|---|
| 1 | actcccaccc taagtgctgc agactcttcc ctgaagctgc cggctgaggc cggagctgcc |
| 61 | gcctccatga gaggcttcct cctacacccc agggccagag gaccctttgc caccagagtg |
| 121 | agatcctaga gaccatcatc ctggtaaatc ccagtgcaga cagcatcagc tctgaggttc |
| 181 | atcatcttct tagcagctca tcagcttata aactactaat cttgagtggg caaagtttag |
| 241 | agcctggggg agacctcatc ctacagagtg gcacctactc atatgaaaac tttgcccagg |
| 301 | tccttcacaa ccccgagatt tcccaattgc tcagcaatag agaccctggg atacaggcct |
| 361 | tccttaccgt gtcctgctta ggggaaggtg attggagcca cctgggatta tccagttccc |
| 421 | aagagaccct gcacctccgg ctaaaccctg agcccactct gcccaccatg gacggcgtgg |
| 481 | ctgagttctc cgagtatgtc tctgagactg tggacgtgcc atccccattt gacctactag |
| 541 | agccccccac ctcagggggc ttcctcaagc tctccaagcc ttgttgctac atcttcccag |
| 601 | gtggtcgtgg ggactctgcc ctctttgctg tcaatggttt caacatcctg gtggatggtg |
| 661 | gctctgatcg caagtcctgt ttttggaagc tggtacggca cttggaccgc attgactcgg |
| 721 | tgctactcac acacattggg gcagacaacc tgccaggcat caatggacta ctgcagcgca |
| 781 | aagtggcaga gctagaggag gagcagtccc agggctctag cagttacagc gactgggtga |
| 841 | agaaccttat ctctcctgag cttggagttg tctttttcaa cgtgcctgag aagctgcggc |
| 901 | ttcctgatgc ctcccggaaa gccaagcgta gcattgagga ggcctgcctc actctgcagc |
| 961 | acttaaaccg cctgggcatc caggctgagc ctctatatcg tgtggtcagc aataccattg |
| 1021 | agccactgac cctcttccac aaaatgggtg tgggccggct ggacatgtat gtcctcaacc |
| 1081 | ctgtcaagga cagcaaggag atgcagttcc tcatgcaaaa gtgggcaggc aatagtaaag |
| 1141 | ccaagacagg catcgtgctg cccaatggga aggaggctga gatctccgtg ccctacctta |
| 1201 | cctctatcac tgctctggtg gtctggctac cagccaatcc cactgagaag attgtgcgtg |
| 1261 | tgctttttcc aggaaatgct ccccaaaaca agatcttgga gggcctagaa aagcttcggc |
| 1321 | atctggactt cctgcgttac cctgtggcca cgcagaagga cctggcttct ggggctgtgc |
| 1381 | ctaccaacct caagcccagc aaaatcaaac agcgggctga tagcaaggag agcctcaaag |
| 1441 | ccactaccaa gacggccgtg agcaagttgg ccaaacggga ggaggtggta gaagagggag |
| 1501 | ccaaggaggc acgttcagag ctggccaagg agttagccaa gacagagaag aaggcaaaag |
| 1561 | agtcatctga gaagccccca gagaagcctg ccaagcctga gagggtgaag acagagtcaa |
| 1621 | gtgaggcact gaaggcagag aagcgaaagc tgatcaaaga caaggtaggg aaaaagcacc |
| 1681 | ttaaagaaaa gatatcaaag ctggaagaaa aaaaagacaa ggagaaaaaa gagatcaaaa |
| 1741 | aggagaggaa agagctcaag aaggatgaag gaaggaagga ggagaagaag gatgccaaga |
| 1801 | aggaggagaa gaggaaagat accaaacctg agctcaagaa gatttccaag ccagacctaa |
| 1861 | agccctttac tcctgaggta cgtaagaccc tctataaagc caaggtccct ggaagagtca |
| 1921 | aaatagacag gagccgtgct atccgtgggg agaaggagct gtcttctgag ccccagacac |
| 1981 | ccccagccca gaagggaact gtaccactcc caaccatcag tgggcacagg gagctggtcc |
| 2041 | tatcctcacc agaggacctc acacaggact ttgaggagat gaagcgtgag gagagggctt |
| 2101 | tgctggctga acaaagggac acaggactag gagataagcc attccctcta gacactgcag |
| 2161 | aggagggacc cccaagtaca gctatccagg gaacaccacc ctctgttcca gggctgggac |
| 2221 | aagaagaaca tgtgatgaag gagaaagagc ttgtcccaga ggtccctgag gaacaaggca |
| 2281 | gcaaggacag aggcctagac tctggggctg aaacagagga agagaaagat acctgggagg |
| 2341 | aaaagaagca gagggaagca gagaggctcc cagacagaac agaagccaga gaggaaagtg |
| 2401 | aacctgaagt aaaggaggat gtgatagaaa aggctgagtt agaagaaatg gaggaggtac |
| 2461 | acccttcaga tgaggaggaa gaggacgcga caaaagctga gggtttttac caaaaacata |
| 2521 | tgcaggaacc cttgaaggta actccaagga gccgggaggc ttttgggggt cgggaattgg |
| 2581 | gactccaggg caaggcccct gagaaggaga cctcgttatt cctaagcagc ctgaccacac |
| 2641 | ctgcaggagc cactgagcat gtctcttaca tccaggatga gacaatccct ggctactcag |
| 2701 | agactgagca gaccatctca gatgaggaga tccatgatga gccggaggag cgcccagctc |
| 2761 | cacccagatt tcatacaagt acatatgacc tgcccgggcc tgaaggtgct ggcccattcg |
| 2821 | aagccagcca acctgccgat agtgctgttc ctgctacctc tggcaaagtc tatggaacgc |
| 2881 | cagagactga actcacctac cccactaaca tagtggctgc ccctttggct gaagaggaac |
| 2941 | atgtgtcctc ggccacttca atcactgagt gtgacaaact ttcttccttt gccacatcag |
| 3001 | tggctgagga ccaatctgtg gcctcactta cagctcccca gacagaggag acaggcaaga |
| 3061 | gctccctgct gcttgacaca gtcacaagca tcccttcctc ccgtactgaa gctacgcagg |
| 3121 | gcttggacta tgtgccatca gctggtacca tctcacccac ctcctcactg gaagaagaca |
| 3181 | agggcttcaa atcaccaccc tgtgaggact tctctgtgac tggggagtca gagaagagag |
| 3241 | gagagatcat agggaaaggc ttgtctggag agagagctgt ggaagaggaa gaggaggaga |
| 3301 | cagcaaacgt agagatgtct gagaaacttt gcagtcaata tggaactcca gtgtttagtg |
| 3361 | cccctgggca tgccctacat ccaggagaac cagcccttgg agaagcagag gagcggtgcc |
| 3421 | ttagcccaga tgacagcaca gtgaagatgg cttctcctcc accatctggc ccacccagtg |
| 3481 | ccacccacac accctttcat cagtccccag tggaagaaaa gtctgagccc caagactttc |
| 3541 | aggaggcaga ctcctgggga gacactaagc gcacaccagg tgtgggcaaa gaagatgctg |
| 3601 | ctgaggagac agtcaagcca gggcctgaag agggcacact agagaaggaa gagaaagttc |
| 3661 | ctcctcccag gagcccccag gcccaggaag cacctgtcaa cattgatgag gggcttacag |
| 3721 | gctgtaccat tcaactgttg ccagcacagg ataaagcaat agtctttgag attatggagg |
| 3781 | caggagagcc cacaggccca attctgggag cagaagccct tcccggaggt ttgaggactt |
| 3841 | taccccaaga acctggcaaa cctcagaaag atgaggtgct cagatatcct gaccgaagcc |
| 3901 | tctctcctga agatgcagaa tccctctctg tcctcagcgt gccctcccca gacactgcca |
| 3961 | accaagagcc tacccccaag tctccctgtg gcctgacaga acagtaccta cacaaagacc |
| 4021 | gttggccaga ggtatctcca gaagacaccc agtcactttc tctgtcagaa gagagtccca |
| 4081 | gcaaggagac ctccctggat gtctcttcta agcagctctc tccagaaagc cttggcaccc |
| 4141 | tccagtttgg ggaactaaac cttgggaagg aagaaatggg gcatctgatg caggccgagg |
| 4201 | atacctctca ccacacagct cccatgtctg ttccagagcc ccatgcagcc acagcgtcac |
| 4261 | ctcccacaga tgggacaact cgatactctg cacagacaga catcacagat gacagccttg |
| 4321 | acaggaagtc acctgccagc tcattctctc actctacacc ttcaggaaat gggaagtact |
| 4381 | tacctggggc gatcacaagc cctgatgaac acattctgac acctgatagc tccttctcca |
| 4441 | agagtcctga gtctttgcca ggccctgcct tggaggacat tgccataaag tgggaagata |
| 4501 | aagttccagg gttgaaagac agaacctcag aacagaagaa ggaacctgag ccaaaggatg |
| 4561 | aagttttaca gcagaaagac aaaactctgg agcacaagga ggtggtagag ccgaaggata |
| 4621 | cagccatcta tcagaaagat gaggctctgc atgtaaagaa tgaggctgtg aaacagcagg |
| 4681 | ataaggcttt agaacaaaag ggcagagact tagagcaaaa agacacagcc ctagaacaga |
| 4741 | aggacaaggc cctggaacca aaagacaaag acttagaaga aaaagacaag gccctggaac |
| 4801 | agaaggataa gattccagaa gagaaagaca aagccttaga acaaaaggat acagccctgg |
| 4861 | aacagaagga caaggccctg gaaccaaaag ataaagactt ggaacaaaag gacagggtcc |
| 4921 | tagaacagaa ggagaagatc ccagaagaga aagacaaagc cttagatcaa aaagtcagaa |
| 4981 | gtgttgaaca taaggctccg gaggacacgg tcgctgaaat gaaggacaga gacctagaac |
| 5041 | agacagacaa agcccctgaa cagaaacacc aggcccagga acaaaaggat aaagtctcag |
| 5101 | aaaagaagga tcaggcctta gaacaaaaat actgggcttt gggacagaag gatgaagccc |
| 5161 | tggaacaaaa cattcaggct ctggaagaga accaccaaac tcaggagcag gagagcctag |
| 5221 | tgcaggagga taaaaccagg aaaccaaaga tgctagagga aaaatcccca gaaaaggtca |
| 5281 | aggccatgga agagaagtta gaagctcttc tggagaagac caaagctctg ggcctggaag |
| 5341 | agagcctagt gcaggagggc agggccagag agcaggaaga aaagtactgg agggggcagg |
| 5401 | atgtggtcca ggagtggcaa gaaacatctc ctaccagaga ggagccggct ggagaacaga |
| 5461 | aagagcttgc cccggcatgg gaggacacat ctcctgagca ggacaatagg tattggaggg |
| 5521 | gcagagagga tgtggccttg gaacaggaca catactggag ggagctaagc tgtgagcgga |
| 5581 | aggtctggtt ccctcacgag ctggatggcc agggggcccg cccacactac actgaggaac |
| 5641 | gggaaagcac tttcctagat gagggcccag atgatgagca agaagtaccc ctgcgggaac |
| 5701 | acgcaacccg gagcccctgg gcctcagact tcaaggattt ccaggaatcc tcaccacaga |
| 5761 | aggggctaga ggtggagcgc tggcttgctg aatcaccagt tgggttgcca ccagaggaag |
| 5821 | aggacaaact gacccgctct ccctttgaga tcatctcccc tccagcttcc ccacctgaga |
| 5881 | tggttggaca aagggttcct tcagccccag gacaagagag tcctatccca gaccctaagc |
| 5941 | tcatgccaca catgaagaat gaacccacta ctccctcatg gctggctgac atcccaccct |
| 6001 | gggtgcccaa ggacagaccc ctcccccctg cacccctctc cccagctcct ggtcccccca |
| 6061 | cacctgcccc ggaatcccat actcctgcac ccttctcttg gggcacagcc gagtatgaca |
| 6121 | gtgtggtggc tgcagtgcag gagggggcag ctgagttgga aggtgggcca tactcccccc |
| 6181 | tggggaagga ctaccgcaag gctgaagggg aaagggaaga agaaggtagg gctgaggctc |
| 6241 | ctgacaaaag ctcacacagc tcaaaggtac cagaggccag caaaagccat gccaccacgg |
| 6301 | agcctgagca gactgagccg gagcagagag agcccacacc ctatcctgat gagagaagct |
| 6361 | ttcagtatgc agacatctat gagcagatga tgcttactgg gcttggccct gcatgcccca |
| 6421 | ctagagagcc tccacttgga gcagctgggg attggccccc atgcctctca accaaggagg |
| 6481 | cagctgccgg ccgaaacaca tctgcagaga aggagctttc atctcctatc tcacccaaga |
| 6541 | gcctccagtc tgacactcca accttcagct atgcagccct ggcaggaccc actgtacccc |
| 6601 | caaggccaga gccagggcca agtatggagc ccagcctcac cccacctgca gttccccccc |
| 6661 | gtgctcctat cctgagcaaa ggcccaagcc cccctcttaa tggtaacatc ctgagctgca |
| 6721 | gcccagatag gaggtcccca tcccccaagg aatcaggccg gagtcactgg gatgacagca |
| 6781 | ctagtgactc agaactggag aagggggctc gggaacagcc agaaaaagag gcccaatccc |
| 6841 | caagtcctcc tcaccccatt cctatggggt cccccacatt atggccagaa actgaggcac |
| 6901 | atgttagccc tcccttggac tcacacctgg ggcctgcccg acccagtctg gacttccctg |
| 6961 | cttcagcctt tggcttctcc tcattgcagc cagctccccc acagctgccc tctccagctg |
| 7021 | aaccccgctc ggcaccctgt ggctcccttg ccttctctgg ggatcgagct ctggctctgg |
| 7081 | ctccaggacc ccccaccaga acccggcatg atgaatacct ggaagtgacc aaggccccca |
| 7141 | gcctggattc ctcactgccc cagctcccat cacccagttc tcctggggcc cctctcctct |
| 7201 | ccaatctgcc acgacctgcc tcaccagccc tgtctgaggg ctcctcctct gaggctacca |
| 7261 | cgcctgtgat ttcaagtgtg gcggagcgct tctctccaag ccttgaggct gcagaacagg |
| 7321 | agtctggaga gctggaccca ggaatggaac cagctgccca cagcctctgg gacctcactc |
| 7381 | ctctgagccc agcaccccca gcttcactgg acttggccct agctccagct ccaagcctgc |
| 7441 | ctggagacat gggtgatggc atcctgccgt gccacctgga gtgctcagag gcagccacgg |
| 7501 | agaagccaag ccccttccag gttccctctg aggattgtgc agccaatggc ccaactgaaa |
| 7561 | ccagccctaa ccccccaggc cctgccccag ccaaggctga aaatgaagag gctgcggctt |
| 7621 | gccctgcctg ggaacgtggg gcctggcctg aaggagctga gaggagctcc cggcctgaca |
| 7681 | cattgctctc ccctgagcag ccagtgtgtc ctgcaggggg ctccgggggc ccacccagca |
| 7741 | gtgcctctcc tgaggtcgaa gctgggcccc agggatgtgc cactgagcct cggccccatc |
| 7801 | gtggggagct ctccccatcc ttcctgaacc cacctctgcc cccatccata gatgataggg |
| 7861 | acctctcaac tgaggaagtt cggctagtag gaagaggggg gcggcgccgg gtaggggggc |
| 7921 | cagggaccac tgggggccca tgccctgtga ctgatgagac accccctaca tcagccagtg |
| 7981 | actcaggctc ctcacagtca gattctgatg tcccgccaga aactgaggag tgtccgtcca |
| 8041 | tcacagctga ggcagccctc gactcagatg aagatggaga cttcctacct gtggacaaag |
| 8101 | ctgggggtgt cagtggtact caccacccca ggcctggcca tgacccacct cctctcccac |
| 8161 | agccagaccc ccgcccatcc cctccccgcc ctgatgtgtg catggctgac cccgaggggc |
| 8221 | tcagctcaga gtctgggaga gtagagaggc tacgggagaa ggaaaaggtt caggggcgag |
| 8281 | tagggcgcag ggccccaggc aaggccaagc cagcgtcccc tgcacggcgt ctggatcttc |
| 8341 | ggggaaaacg ctcacccacc cctggtaaag ggcctgcaga tcgagcatcc cgggccccac |
| 8401 | ctcgaccacg cagcaccaca agccaggtca ccccagcaga ggaaaaggat ggacacagcc |
| 8461 | ccatgtccaa aggcctagtc aatggactca aggcaggacc aatggccttg agttccaagg |
| 8521 | gcagctctgg tgcccctgta tatgtggatc tcgcctacat cccgaatcat tgcagtggca |
| 8581 | agactgctga ccttgacttc ttccgtcgag tgcgtgcatc ctactatgtg gtcagtggga |
| 8641 | atgaccctgc caatggcgag ccaagccggg ctgtgctgga tgccctgctg gagggcaagg |
| 8701 | cccagtgggg ggagaatctt caggtgactc tgatccctac tcatgacacg gaggtgactc |
| 8761 | gtgagtggta ccaacaaact catgagcagc agcaacaact gaatgtcctg gtcctggcta |
| 8821 | gcagcagcac cgtggtgatg caggatgagt ccttccctgc ctgcaagatt gagttctgaa |
| 8881 | agagccgccc tcccttcccc aaggatccac tcccccagct cctttagaga atggctactg |
| 8941 | ctgagtcctt tggggttgag ggagatggga gctaggggga ggggagggag atgtcttgtt |
| 9001 | gtggggactt gggctgggct aaatgggagg ggttgtccct ccccatcatc cattcctgtg |
| 9061 | aggtgtctca aaccaaagtt aacagggaga ggatggggga ggggacaaat tagaatagga |
| 9121 | tagcatctga tgcctgagaa ccctctccta gcactgtcaa atgctggtat tgaatgggga |
| 9181 | ctgaggatgg gtctcagaga gcaacctcct ccctcgtaga gggagattat atccccaact |
| 9241 | ccagggacct ctttatctca atctatttat ttggcatcct gggagggatt tccaatagta |
| 9301 | atttatgtga cctggggcag gataccgtca gtgaggtgcc cagagctgca ccctttcctc |
| 9361 | catttcccat cccccatctc ctcaaccacc agggtctgag ttctagcagg gtcctggggg |
| 9421 | tatcccactg ctatactgtt ctactgcttc cctcagtatc tgaatgtctc aatttaaaac |
| 9481 | ttgaagctct ttagaccaat agactggtga gaggagaaag gagcttatcc cccagaccct |
| 9541 | gctttatacc attcacatcc cagggctgtg tccagacagc acaaaacggc aaggagagcc |
| 9601 | caagccccaa tgccagaatt cttccaaact ccctgactct ttgaagtttt tactcacccc |
| 9661 | atttcaatta tcctgatccc ttctcatccc ctgcttggct tctctgcatg tggtcatctg |
| 9721 | ctgtggcttg gtgtttaatg ggttaaaaat aagccactgc ctgacatccc aacatttgac |
| 9781 | accccagcaa tgtgtgactc ccccaacatt ccactatgcc atcctgcagc tgaaatggga |
| 9841 | acactggctg cctctccaaa cccgctcttg gacagaggat ctgggaggtg gaagccaggc |
| 9901 | cagaggactt ggggaaaatg agatggagga aggaaaaagg gagaagctga gccacagctt |
| 9961 | aactcctaca gagtgaaatg aaaacgggct gaaaatacca ccccaggaga ggacctcgcc |
| 10021 | ccaagcaagc cagtgagcag ccctgccaga ctactgccag actgagaaac ccagaagctg |
| 10081 | gtagtcatgt gggcttgcct tctctgccaa acgactggga aaccaaaatg agcccacctt |
| 10141 | gtgttcttcc tagctccacc ctccccgtgc tgctgtgttc tgctcctccc cacgcttccc |
| 10201 | tgctatagtt cccagctgct gtaacggagc cacctccaac tctaacaata aaccaagttc |
| 10261 | attgcagata gtgta |

SELP (SEQ ID NO: 51) - Homo sapiens selectin P (SELP), mRNA -
NM_003005

| | |
|---|---|
| 1 | ggcagtgaga ctgtaagcag tctgggttgg gcagaaggca gaaaaccagc agagtcacag |
| 61 | aggagatggc caactgccaa atagccatct tgtaccagag attccagaga gtggtctttg |
| 121 | gaatttccca actcctttgc ttcagtgccc tgatctctga actaacaaac cagaaagaag |
| 181 | tggcagcatg gacttatcat tacagcacaa aagcatactc atggaatatt tcccgtaaat |
| 241 | actgccagaa tcgctacaca gacttagtgg ccatccagaa taaaaatgaa attgattacc |
| 301 | tcaataaggt cctaccctac tacagctcct actactggat tgggatccga aagaacaata |
| 361 | agacatggac atgggtggga accaaaaagg ctctcaccaa cgaggctgag aactgggctg |
| 421 | ataatgaacc taacaacaaa aggaacaacg aggactgcgt ggagatatac atcaagagtc |
| 481 | cgtcagcccc tggcaagtgg aatgatgagc actgcttgaa gaaaaagcac gcattgtgtt |
| 541 | acacagcctc ctgccaggac atgtcctgca gcaaacaagg agagtgcctc gagaccatcg |
| 601 | ggaactacac ctgctcctgt taccctggat tctatgggcc agaatgtgaa tacgtgagag |
| 661 | agtgtggaga acttgagctc cctcaacacg tgctcatgaa ctgcagccac cctctgggaa |
| 721 | acttctcttt taactcgcag tgcagcttcc actgcactga cgggtaccaa gtaaatgggc |
| 781 | ccagcaagct ggaatgcttg gcttctggaa tctggacaaa taagcctcca cagtgtttag |
| 841 | ctgcccagtg cccacccctg aagattcctg aacgaggaaa catgacctgc cttcattctg |
| 901 | caaaagcatt ccagcatcag tctagctgca gcttcagttg tgaagaggga tttgcattag |
| 961 | ttggaccgga agtggtgcaa tgcacagcct cgggggtatg gacagcccca gccccagtgt |
| 1021 | gtaaagctgt gcagtgtcag cacctggaag cccccagtga aggaaccatg gactgtgttc |
| 1081 | atccgctcac tgcttttgcc tatggctcca gctgtaaatt tgagtgccag cccggctaca |
| 1141 | gagtgagggg cttggacatg ctccgctgca ttgactctgg acactggtct gcacccttgc |
| 1201 | caacctgtga ggctatttcg tgtgagccgc tggagagtcc tgtccacgga agcatggatt |
| 1261 | gctctccatc cttgagagcg tttcagtatg acaccaactg tagcttccgc tgtgctgaag |
| 1321 | gtttcatgct gagaggagcc gatatagttc ggtgtgataa cttgggacag tggacagcac |
| 1381 | cagccccagt ctgtcaagct ttgcagtgcc aggatctccc agttccaaat gaggcccggg |
| 1441 | tgaactgctc ccaccccttc ggtgccttta ggtaccagtc agtctgcagc ttcacctgca |
| 1501 | atgaaggctt gctcctggtg ggagcaagtg tgctacagtg cttggctact ggaaactgga |
| 1561 | attctgttcc tccagaatgc caagccattc cctgcacacc tttgctaagc cctcagaatg |
| 1621 | gaacaatgac ctgtgttcaa cctcttggaa gttccagtta taaatccaca tgtcaattca |
| 1681 | tctgtgacga gggatattct ttgtctggac cagaaagatt ggattgtact cgatcgggac |
| 1741 | gctggacaga ctccccacca atgtgtgaag ccatcaagtg cccagaactc tttgccccag |
| 1801 | agcagggcag cctggattgt tctgacactc gtggagaatt caatgttggc tccacctgcc |
| 1861 | atttctcttg tgacaacggc tttaagctgg aggggcccaa taatgtggaa tgcacaactt |
| 1921 | ctggaagatg gtcagctact ccaccaacct gcaaaggcat agcatcactt cctactccag |
| 1981 | gggtgcaatg tccagccctc accactcctg ggcagggaac catgtactgt aggcatcatc |
| 2041 | cgggaacctt tggttttaat accacttgtt actttggctg caacgctgga ttcacactca |
| 2101 | taggagacag cactctcagc tgcagacctt caggacaatg gacagcagta actccagcat |
| 2161 | gcagagctgt gaaatgctca gaactacatg ttaataagcc aatagcgatg aactgctcca |
| 2221 | acctctgggg aaacttcagt tatggatcaa tctgctcttt ccattgtcta gagggccagt |
| 2281 | tacttaatgg ctctgcacaa acagcatgcc aagagaatgg ccactggtca actaccgtgc |
| 2341 | caacctgcca agcaggacca ttgactatcc aggaagccct gacttacttt ggtggagcgg |
| 2401 | tggcttctac gataggtctg ataatgggtg ggacgctcct ggctttgcta agaaagcgtt |
| 2461 | tcagacaaaa agatgatggg aaatgcccct tgaatcctca cagccaccta ggaacatatg |
| 2521 | gagtttttac aaacgctgca tttgacccga gtccttaagg tttccataaa cacccatgaa |
| 2581 | tcaaagacat ggaattacct tagattagct ctggaccagc ctgttggacc cgctctggac |
| 2641 | caaccctgtt tcctgagttt gggattgtgg tacaatctca aattctcaac ctaccacccc |
| 2701 | ttcctgtccc acctcttctc ttcctgtaac acaagccaca gaagccagga gcaaatgttt |
| 2761 | ctgcagtagt ctctgtgctt tgactcacct gttacttgaa ataccagtga accaaagaga |
| 2821 | ctggagcatc tgactcacaa gaagaccaga ctgtggagaa ataaaaatac ctctttattt |
| 2881 | tttgattgaa ggaaggtttt ctccactttg ttggaaagca ggtggcatct ctaattggaa |
| 2941 | gaaattcctg tagcatcttc tggagtctcc agtggttgct gttgatgagg cctcttggac |
| 3001 | ctctgctctg aggcttccag agagtcctct ggatggcacc agaggctgca gaaggccaag |
| 3061 | aatcaagcta gaaggccaca tgtcaccgtg gaccttcctg ccaccagtca ctgtccctca |
| 3121 | aatgacccaa agaccaatat tcaaatgcgt aattaaaaga attttcccca aaaaaaaaaa |
| 3181 | aaaaa |

NEXN (SEQ ID NO: 52) nexilin F-actin binding protein, transcript
variant 1, mRNA NM_144573.3

| | |
|---|---|
| 1 | aacagctgca gccggcgctg ggcccgcctg gaatgcggga acaggctgca caccaggact |
| 61 | tttatggaaa cttgctgctg gagacggcgg cggcggcggc ggcagcggca gccagaggac |
| 121 | tcccagcggc tggagcagaa gtgttagcgg ccagagctcc cagaccccta cccacagcca |
| 181 | ggcgggacgc gcacagtccc tccacgcgga aagaagtacc ttcgccggtc accggctcct |
| 241 | gcagggtgca aatatataca gagcttcata atcagcccaa gaccacatag agcaaacatg |
| 301 | aatgatattt cccaaaaggc tgagattctg ctttcttcat ctaaacctgt cccaaaaacc |
| 361 | tatgtaccaa aacttggcaa gggtgatgta aaggataagt ttgaagccat gcagagagcc |
| 421 | agggaagaaa gaaatcaaag gagatctaga gacgaaaaac aaagaagaaa agaacaatat |
| 481 | attagagaga gagaatggaa caggagaaag caggagatta aagaaatgct tgcttctgat |
| 541 | gatgaggaag atgtatcttc taaagtagaa aaggcttatg ttccaaaatt aacaggaact |
| 601 | gtgaagggta gatttgctga aatggagaaa caaagacaag aggaacaaag gaagagaacg |
| 661 | gaggaggaac gaaaacgcag aattgagcag gatatgttag aaaagaggaa aatacagcgt |
| 721 | gaattagcaa aaagggctga acagattgag gacataaaca atacgggaac tgaatcagca |
| 781 | tcagaggaag gagatgattc actacttata actgtggtac ctgtcaaatc atataaaaca |
| 841 | tctggaaaaa tgaaaaagaa ttttgaggat ctagaaaaag aacgtgaaga gaaagaaagg |
| 901 | atcaagtacg aggaagataa aagaataaga tatgaagaac aacgaccatc tctcaaggaa |
| 961 | gcaaagtgtc tttcattagt tatggatgat gaaatagaaa gtgaagcaaa aaaagaatca |
| 1021 | ctttctcccg gaaaattgaa actaactttt gaagaactgg agcgacaaag acaagaaaac |
| 1081 | cgaaagaagc aagctgaaga ggaagcaaga aaacgtttag aagaagagaa gcgtgctttt |
| 1141 | gaagaagcaa ggcggcaaat ggtaaatgaa gatgaggaaa accaagacac agcaaaaatt |
| 1201 | tttaaagggt accgccctgg taaactcaaa ctcagttttg aagaaatgga aaggcaaaga |
| 1261 | agagaagatg aaaaaaggaa agcagaagaa gaagccagaa ggagaataga ggaagaaaag |
| 1321 | aaggcgtttg ctgaagcaag gagaaatatg gtagtagatg atgactcccc agagatgtat |
| 1381 | aagacaatct ctcaagaatt tcttacaccg ggaaaactgg aaattaattt tgaagaatta |
| 1441 | ttaaaacaaa aaatggaaga agaaaaacga cgaacagagg aggaacggaa gcataagcta |
| 1501 | gaaatggaga aacaagaatt tgaacaactg agacaggaaa tgggagagga agaggaagaa |
| 1561 | aatgaaacct ttggattgag cagagaatat gaagaactga tcaaattaaa aaggagtggc |
| 1621 | tctattcaag ctaaaaacct aaaaagcaag tttgaaaaaa ttggacagtt gtctgaaaaa |
| 1681 | gaaatacaga aaaaaataga agaagagcga gcaagaagga gagcaattga ccttgaaatt |
| 1741 | aaagagcgag aagctgaaaa ttttcatgag gaagatgatg ttgatgttag gcctgcaaga |
| 1801 | aaaagcgagg ctccatttac tcacaaagtg aatatgaaag ctagatttga acaaatggct |
| 1861 | aaggcaagag aagaagaaga acaaagaaga attgaagaac aaaagttact acgcatgcag |
| 1921 | tttgaacaaa gggaaattga tgcagcacta caaaagaaaa gagaagagga ggaggaggaa |
| 1981 | gaaggtagca tcatgaatgg ctccactgct gaagatgaag agcaaaccag atcaggagct |
| 2041 | ccatggttca agaagcctct taaaaacaca tcagttgtag acagtgagcc agtcagattt |
| 2101 | acggttaaag taacaggaga acccaaacca gaaattacat ggtggtttga aggagaaata |
| 2161 | ctgcaggatg gagaagacta tcaatatatt gaaaggggag aaacttactg cctttactta |
| 2221 | ccagaaactt tcccagaaga tggaggagag tatatgtgta aagcagtcaa caataaagga |
| 2281 | tctgcagcta gtacctgtat tcttaccatt gaaagtaaga attaatcact ctttttatct |
| 2341 | tttattctat taattttttt ttccttaaaa tcacttttct tcttctcttt tttagctgat |
| 2401 | gactactagc tcccctcccc tctccctgga actttctctt tcactccaac tttcttacta |
| 2461 | catccatctt ttctgtggcg gggccaaaaa aggaaaccag gagtgccact atgctgactt |
| 2521 | cttattcctt ttcataacag tcttcaaagc acagctcatc taaagaatgc ctacttcttt |
| 2581 | tccaaataag catcagattt atcgcctatt atgcagtaac agtcaataaa atgtacttat |
| 2641 | gggggggaat tactcaatta ttctatcaga acctattata aagactgtat ttcccataga |
| 2701 | cgtttacagc aactatgttt aaaaaacaaa aacaaaaaaa aaacacacaa acctaagtag |
| 2761 | aatacattat tttgcatgaa ggaatgtcat ttctgagctt tttacaccta aaattaggct |
| 2821 | gaaatagctg agataattaa tttggaacct atcaatttga gtggactttt tctttagtag |
| 2881 | tacaccattt tggttgttgt agtttcaaag tctttctgaa gcagatatat tgggattgga |
| 2941 | gcggggtggg gaaaactgtc actcctttca gaggaaaagg ggaggagcat ggagaaaaac |
| 3001 | aaaaattaaa ggacttaaag aatggctata cagtgttgag tgttgaggat attaaacatg |
| 3061 | ttatttttca aacgtatgta atatatatta aatttataaa gcaaatttat gttgtgatct |
| 3121 | tgcctgaaca aattatattt taatgaaaaa actttctatt aatagttcac gcaagagaaa |
| 3181 | acactttcaa catagtcgaa ggcttcaaga tctaagtgta tcagacttag ggaaaaagtg |
| 3241 | gcacaacctt cgatttaaaa ttctagtctt taaaatgagt ttgtaaataa ttagctatta |
| 3301 | cgttctatta agttgtttta tattttaatt ttctggaaga caattttatt ttacaacgtg |
| 3361 | aacccaaata aagtaacttc tgtatttaaa aaaaaaaaaa aaaaa |

ITGA2B (SEQ ID NO: 53) - Homo sapiens integrin subunit alpha 2b
(ITGA2B), mRNA - NM_000419

| | |
|---|---|
| 1 | gctctgcccg ttgctcagca agttacttgg ggttccagtt tgataagaaa agacttcctg |
| 61 | tggaggaatc tgaagggaag gaggaggagc tggcccattc ctgcctggga ggttgtggaa |
| 121 | gaaggaagat ggccagagct ttgtgtccac tgcaagccct ctggcttctg gagtgggtgc |
| 181 | tgctgctctt gggaccttgt gctgcccctc cagcctgggc cttgaacctg gacccagtgc |
| 241 | agctcacctt ctatgcaggc cccaatggca gccagtttgg attttcactg gacttccaca |
| 301 | aggacagcca tgggagagtg gccatcgtgg tgggcgcccc gcggaccctg ggccccagcc |
| 361 | aggaggagac gggcggcgtg ttcctgtgcc cctggagggc cgagggcggc cagtgcccct |
| 421 | cgctgctctt tgacctccgt gatgagaccc gaaatgtagg ctcccaaact ttacaaacct |
| 481 | tcaaggcccg ccaaggactg ggggcgtcgg tcgtcagctg gagcgacgtc attgtggcct |
| 541 | gcgccccctg gcagcactgg aacgtcctag aaaagactga ggaggctgag aagacgcccg |
| 601 | taggtagctg ctttttggct cagccagaga gcggccgccg cgccgagtac tccccctgtc |
| 661 | gcgggaacac cctgagccgc atttacgtgg aaaatgattt tagctgggac aagcgttact |
| 721 | gtgaagcggg cttcagctcc gtggtcactc aggccggaga gctggtgctt ggggctcctg |
| 781 | gcggctatta tttcttaggt ctcctggccc aggctccagt tgcggatatt ttctcgagtt |
| 841 | accgcccagg catccttttg tggcacgtgt cctcccagag cctctccttt gactccagca |
| 901 | acccagagta cttcgacggc tactgggggt actcggtggc cgtgggcgag ttcgacgggg |
| 961 | atctcaacac tacagaatat gtcgtcggtg cccccacttg gagctggacc ctgggagcgg |
| 1021 | tggaaatttt ggattcctac taccagaggc tgcatcggct gcgcggagag cagatggcgt |
| 1081 | cgtattttgg gcattcagtg gctgtcactg acgtcaacgg ggatgggagg catgatctgc |
| 1141 | tggtgggcgc tccactgtat atggagagcc gggcagaccg aaaactggcc gaagtggggc |
| 1201 | gtgtgtattt gttcctgcag ccgcgaggcc cccacgcgct gggtgccccc agcctcctgc |
| 1261 | tgactggcac acagctctat gggcgattcg gctctgccat cgcacccctg ggcgacctcg |
| 1321 | accgggatgg ctacaatgac attgcagtgg ctgcccccta cgggggtccc agtggccggg |
| 1381 | gccaagtgct ggtgttcctg ggtcagagtg aggggctgag gtcacgtccc tcccaggtcc |
| 1441 | tggacagccc cttccccaca ggctctgcct ttggcttctc ccttcgaggt gccgtagaca |
| 1501 | tcgatgacaa cggataccca gacctgatcg tgggagctta cggggccaac caggtggctg |
| 1561 | tgtacagagc tcagccagtg gtgaaggcct ctgtccagct actggtgcaa gattcactga |
| 1621 | atcctgctgt gaagagctgt gtcctacctc agaccaagac acccgtgagc tgcttcaaca |
| 1681 | tccagatgtg tgttggagcc actgggcaca acattcctca gaagctatcc ctaaatgccg |
| 1741 | agctgcagct ggaccggcag aagccccgcc agggccggcg ggtgctgctg ctgggctctc |
| 1801 | aacaggcagg caccaccctg aacctggatc tgggcggaaa gcacagcccc atctgccaca |
| 1861 | ccaccatggc cttccttcga gatgaggcag acttccggga caagctgagc cccattgtgc |
| 1921 | tcagcctcaa tgtgtcccta ccgcccacgg aggctggaat ggcccctgct gtcgtgctgc |
| 1981 | atggagacac ccatgtgcag gagcagacac gaatcgtcct ggactgtggg gaagatgacg |
| 2041 | tatgtgtgcc ccagcttcag ctcactgcca gcgtgacggg ctccccgctc ctagttgggg |
| 2101 | cagataatgt cctggagctg cagatggacg cagccaacga gggcgagggg gcctatgaag |
| 2161 | cagagctggc cgtgcacctg ccccagggcg cccactacat gcgggcccta agcaatgtcg |
| 2221 | agggctttga gagactcatc tgtaatcaga agaaggagaa tgagaccagg gtggtgctgt |
| 2281 | gtgagctggg caaccccatg aagaagaacg cccagatagg aatcgcgatg ttggtgagcg |
| 2341 | tggggaatct ggaagaggct ggggagtctg tgtccttcca gctgcagata cggagcaaga |
| 2401 | acagccagaa tccaaacagc aagattgtgc tgctggacgt gccggtccgg gcagaggccc |
| 2461 | aagtggagct gcgagggaac tcctttccag cctccctggt ggtggcagca gaagaaggtg |
| 2521 | agagggagca gaacagcttg gacagctggg gacccaaagt ggagcacacc tatgagctcc |
| 2581 | acaacaatgg ccctgggact gtgaatggtc ttcacctcag catccacctt ccgggacagt |
| 2641 | cccagccctc cgacctgctc tacatcctgg atatacagcc ccaggggggc cttcagtgct |
| 2701 | tcccacagcc tcctgtcaac cctctcaagg tggactgggg gctgcccatc cccagcccct |
| 2761 | cccccattca cccggcccat cacaagcggg atcgcagaca gatcttcctg ccagagcccg |
| 2821 | agcagccctc gaggcttcag gatccagttc tcgtaagctg cgactcggcg ccctgtactg |
| 2881 | tggtgcagtg tgacctgcag gagatggcgc gcgggcagcg ggccatggtc acggtgctgg |
| 2941 | ccttcctgtg gctgcccagc ctctaccaga ggcctctgga tcagtttgtg ctgcagtcgc |
| 3001 | acgcatggtt caacgtgtcc tccctcccct atgcggtgcc cccgctcagc ctgccccgag |
| 3061 | gggaagctca ggtgtggaca cagctgctcc gggccttgga ggagagggcc attccaatct |
| 3121 | ggtgggtgct ggtgggtgtg ctgggtggcc tgctgctgct caccatcctg gtcctggcca |
| 3181 | tgtggaaggt cggcttcttc aagcggaacc ggccacccct ggaagaagat gatgaagagg |
| 3241 | gggagtgatg gtgcagccta cactattcta gcaggagggt tgggcgtgct acctgcaccg |
| 3301 | ccccttctcc aacaagttgc ctccaagctt tgggttggag ctgttccatt gggtcctctt |
| 3361 | ggtgtcgttt ccctcccaac agagctgggc taccccccct cctgctgcct aataaagaga |
| 3421 | ctgagccctg aaaaaaaaaa aaaaaaaaa |

MYL9 (SEQ ID NO: 54) - Homo sapiens myosin light chain 9 (MYL9),
transcript variant 2, mRNA - NM_181526

| | |
|---|---|
| 1 | gcccccgcct ggagtccaga cccgacggcc ggcccagttc cacgcaccca gcgagcccaa |
| 61 | gcgccttctc cgcaccaggg aagccccacc caccagaagc caagatgtcc agcaagcggg |
| 121 | ccaaagccaa gaccaccaag aagcggccac agcgggccac atccaatgtc ttcgcaatgt |
| 181 | ttgaccagtc ccagatccag gagtttaagg aggctttcaa catgattgac cagaaccgtg |
| 241 | atggcttcat tgacaaggag gacctgcacg acatgctggc ctcgctgggt ttcatccatg |
| 301 | aggaccacct ccgggagctg ctcaccacca tgggtgaccg cttcacagat gaggaagtgg |
| 361 | acgagatgta ccgggaggca cccattgata agaaaggcaa cttcaactac gtggagttca |
| 421 | cccgcatcct caaacatggc gccaaggata aagacgacta ggccacccca gccccctgac |
| 481 | accccagccc ccgccagtca cccctccccg cacacacccg tccataccag ctccctgccc |
| 541 | atgaccctcg ctcagggatc cccctttgag gggttagggt cccagttccc agtggaagaa |
| 601 | acaggccagg agaagtgcgt gccgagctga ggcagatgtt cccacagtga ccccagagcc |
| 661 | ctgggctata gtctctgacc cctccaagga aagaccacct tctggggaca tgggctggag |
| 721 | ggcaggacct agaggcacca agggaaggcc ccattccggg gctgttcccc gaggaggaag |
| 781 | ggaaggggct ctgtgtgccc cccaggagga agaggccctg agtcctggga tcagacaccc |
| 841 | cttcacgtgt atccccacac aaatgcaagc tcaccaaggt cccctctcag tccccttccc |
| 901 | tacaccctga ccggccactg ccgcacaccc acccagagca cgccacccgc catgggagtg |
| 961 | tgctcaggag tcgcgggcag cgtggacatc tgtcccagag ggggcagaat ctccaataga |
| 1021 | ggactgagca ctgctaaaaa aaaaaaaaaa aaaa |

ITGB3 (SEQ ID NO: 55) integrin subunit beta 3, NM_000212.2

| | |
|---|---|
| 1 | cgccgcggga ggcggacgag atgcgagcgc ggccgcggcc ccggccgctc tgggcgactg |
| 61 | tgctggcgct gggggcgctg gcgggcgttg gcgtaggagg gcccaacatc tgtaccacgc |
| 121 | gaggtgtgag ctcctgccag cagtgcctgg ctgtgagccc catgtgtgcc tggtgctctg |
| 181 | atgaggccct gcctctgggc tcacctcgct gtgacctgaa ggagaatctg ctgaaggata |
| 241 | actgtgcccc agaatccatc gagttcccag tgagtgaggc ccgagtacta gaggacaggc |
| 301 | ccctcagcga caagggctct ggagacagct cccaggtcac tcaagtcagt ccccagagga |
| 361 | ttgcactccg gctccggcca gatgattcga agaatttctc catccaagtg cggcaggtgg |
| 421 | aggattaccc tgtggacatc tactacttga tggacctgtc ttactccatg aaggatgatc |
| 481 | tgtggagcat ccagaacctg ggtaccaagc tggccaccca gatgcgaaag ctcaccagta |
| 541 | acctgcggat tggcttcggg gcatttgtgg acaagcctgt gtcaccatac atgtatatct |
| 601 | ccccaccaga ggccctcgaa aacccctgct atgatatgaa gaccacctgc ttgcccatgt |
| 661 | ttggctacaa acacgtgctg acgctaactg accaggtgac ccgcttcaat gaggaagtga |
| 721 | agaagcagag tgtgtcacgg aaccgagatg ccccagaggg tggctttgat gccatcatgc |
| 781 | aggctacagt ctgtgatgaa aagattggct ggaggaatga tgcatcccac ttgctggtgt |
| 841 | ttaccactga tgccaagact catatagcat tggacggaag gctggcaggc attgtccagc |
| 901 | ctaatgacgg gcagtgtcat gttggtagtg acaatcatta ctctgcctcc actaccatgg |
| 961 | attatccctc tttggggctg atgactgaga agctatccca gaaaaacatc aatttgatct |
| 1021 | ttgcagtgac tgaaaatgta gtcaatctct atcagaacta tagtgagctc atcccaggga |
| 1081 | ccacagttgg ggttctgtcc atggattcca gcaatgtcct ccagctcatt gttgatgctt |
| 1141 | atgggaaaat ccgttctaaa gtagagctgg aagtgcgtga cctccctgaa gagttgtctc |
| 1201 | tatccttcaa tgccacctgc ctcaacaatg aggtcatccc tggcctcaag tcttgtatgg |
| 1261 | gactcaagat tggagacacg gtgagcttca gcattgaggc caaggtgcga ggctgtcccc |
| 1321 | aggagaagga gaagtccttt accataaagc ccgtgggctt caaggacagc ctgatcgtcc |
| 1381 | aggtcacctt tgattgtgac tgtgcctgcc aggcccaagc tgaacctaat agccatcgct |
| 1441 | gcaacaatgg caatgggacc tttgagtgtg gggtatgccg ttgtgggcct ggctggctgg |
| 1501 | gatcccagtg tgagtgctca gaggaggact atcgcccttc ccagcaggac gaatgcagcc |
| 1561 | cccgggaggg tcagcccgtc tgcagccagc ggggcgagtg cctctgtggt caatgtgtct |
| 1621 | gccacagcag tgactttggc aagatcacgg gcaagtactg cgagtgtgac gacttctcct |
| 1681 | gtgtccgcta caagggggag atgtgctcag gccatggcca gtgcagctgt ggggactgcc |
| 1741 | tgtgtgactc cgactggacc ggctactact gcaactgtac cacgcgtact gacacctgca |
| 1801 | tgtccagcaa tgggctgctg tgcagcggcc gcggcaagtg tgaatgtggc agctgtgtct |
| 1861 | gtatccagcc gggctcctat ggggacacct gtgagaagtg ccccacctgc ccagatgcct |
| 1921 | gcacctttaa gaaagaatgt gtggagtgta agaagtttga ccggggagcc ctacatgacg |
| 1981 | aaaatacctg caaccgttac tgccgtgacg agattgagtc agtgaaagag cttaaggaca |
| 2041 | ctggcaagga tgcagtgaat tgtacctata agaatgagga tgactgtgtc gtcagattcc |
| 2101 | agtactatga agattctagt ggaaagtcca tcctgtatgt ggtagaagag ccagagtgtc |
| 2161 | ccaagggccc tgacatcctg gtggtcctgc tctcagtgat gggggccatt ctgctcattg |
| 2221 | gccttgccgc cctgctcatc tggaaactcc tcatcaccat ccacgaccga aaagaattcg |
| 2281 | ctaaatttga ggaagaacgc gccagagcaa aatgggacac agccaacaac ccactgtata |
| 2341 | aagaggccac gtctaccttc accaatatca cgtaccgggg cacttaatga taagcagtca |
| 2401 | tcctcagatc attatcagcc tgtgccacga ttgcaggagt ccctgccatc atgtttacag |
| 2461 | aggacagtat ttgtggggag ggatttgggg ctcagagtgg ggtaggttgg gagaatgtca |
| 2521 | gtatgtggaa gtgtgggtct gtgtgtgtgt atgtgggggt ctgtgtgttt atgtgtgtgt |
| 2581 | gttgtgtgtg ggagtgtgta atttaaaatt gtgatgtgtc ctgataagct gagctcctta |
| 2641 | gcctttgtcc cagaatgcct cctgcaggga ttcttcctgc ttagcttgag ggtgactatg |
| 2701 | gagctgagca ggtgttcttc attacctcag tgagaagcca gctttcctca tcaggccatt |
| 2761 | gtccctgaag agaagggcag ggctgaggcc tctcattcca gaggaaggga caccaagcct |
| 2821 | tggctctacc ctgagttcat aaatttatgg ttctcaggcc tgactctcag cagctatggt |
| 2881 | aggaactgct gggcttggca gcccgggtca tctgtacctc tgcctccttt cccctccctc |
| 2941 | aggccgaagg aggagtcagg gagagctgaa ctattagagc tgcctgtgcc ttttgccatc |
| 3001 | ccctcaaccc agctatggtt ctctcgcaag ggaagtcctt gcaagctaat tctttgacct |
| 3061 | gttgggagtg aggatgtctg ggccactcag gggtcattca tggcctgggg gatgtaccag |
| 3121 | catctcccag ttcataatca caacccttca gatttgcctt attggcagct ctactctgga |
| 3181 | ggtttgttta gaagaagtgt gtcaccctta ggccagcacc atctctttac ctcctaattc |
| 3241 | cacaccctca ctgctgtaga catttgctat gagctgggga tgtctctcat gaccaaatgc |
| 3301 | ttttcctcaa agggagagag tgctattgta gagccagagg tctggcccta tgcttccggc |
| 3361 | ctcctgtccc tcatccatag cacctccaca tacctggccc tgtgccttgg tgtgctgtat |
| 3421 | ccatccatgg ggctgattgt atttaccttc tacctcttgg ctgccttgtg aaggaattat |
| 3481 | tcccatgagt tggctgggaa taagtgccag gatggaatga tgggtcagtt gtatcagcac |
| 3541 | gtgtggcctg ttcttctatg ggttggacaa cctcatttta actcagtctt taatctgaga |
| 3601 | ggccacagtg caattttatt ttatttttct catgatgagg ttttcttaac ttaaaagaac |
| 3661 | atgtatataa acatgcttgc attatatttg taaatttatg tgatggcaaa gaaggagagc |
| 3721 | ataggaaacc acacagactt gggcagggta cagacactcc cacttggcat cattcacagc |
| 3781 | aagtcactgg ccagtggctg gatctgtgag gggctctctc atgatagaag gctatgggga |
| 3841 | tagatgtgtg gacacattgg acctttcctg aggaagaggg actgttcttt tgtcccagaa |
| 3901 | aagcagtggc tccattggtg ttgacataca tccaacatta aaagccaccc ccaaatgccc |
| 3961 | aagaaaaaaa gaaagactta tcaacatttg ttccatgagc agaaaactgg agctctggcc |
| 4021 | tcagtgttac agctaaataa tctttaatta aggcaagtca ctttcttctt cttaaagctg |
| 4081 | ttttctagtt tgagaaatga tgggatttta gcagccagtc ttgaaggtct ctttcagtat |
| 4141 | caacattcta agatgctggg acttactgtg tcatcaaatg tgcggttaag attctctggg |
| 4201 | atattgatac tgtttgtgtt tttagttggg agatctgaga gacctggctt tggcaagagc |
| 4261 | agatgtcatt ccatatcacc tttctcaatg aaagtctcat tctatcctct ctccaaaccc |
| 4321 | gttttccaac atttgttaat agttacgtct ctcctgatgt agcacttaag cttcatttag |
| 4381 | ttattatttc tttcttcact ttgcacacat ttgcatccac atattaggga agaggaatcc |
| 4441 | ataagtagct gaaatatcta ttctgtatta ttgtgttaac attgagaata agccttggaa |
| 4501 | ttagatatgg ggcaatgact gagccctgtc tcacccatgg attactcctt actgtaggga |
| 4561 | atggcagtat ggtagaggga taaatagggg gcggggaggg atagtcatgg atccaagaag |
| 4621 | tccttagaaa tagtggcagg gaacaggtgt ggaagctcat gcctgtaatt ataaccttca |
| 4681 | gctactaaga caggtgtggt ggctcacgcc tgtgattata atcttcagtt actaagacag |
| 4741 | agtccatgag agtgttaatg ggacattttc tttagataag atgttttata tgaagaaact |
| 4801 | gtatcaaagg gggaagaaaa tgtatttaac aggtgaatca aatcaggaat cttgtctgag |
| 4861 | ctactggaat gaagttcaca ggtcttgaag acca |

CMTM5 (SEQ ID NO: 56) - Homo sapiens CKLF like MARVEL transmembrane
domain containing 5 (CMTM5), transcript variant 3, mRNA -
NM_001037288

| | |
|---|---|
| 1 | gttagagaag ggggacacaa atgtcttcag gcttaggtcc ctgggggctc ccagtcctgc |
| 61 | ccctgttcct ctactcacat cccagctcct cccagtttct tttcggacct cctcctctcc |
| 121 | ccttccttct ctattccagg ctgggttggg ctctaagcaa ggggagggat tagagcctcc |
| 181 | ttcctctctg cccctcccca tgggtctcta gggggctggt gcaggcagca gcagaggcac |
| 241 | tctgggcagc tgggtgaggg cccatctggg caaggccccc agcgcctgcc ttctctcccg |
| 301 | gggccctgtg ggcaagcctc ctgcttcact ttcaggtttc tcgaagtgcc ttcttgctcc |
| 361 | tgtctgtttc cccatcctgc cagatttctg tttctcttgc tgggcttttg gcagtagggg |
| 421 | gctgtgttgg tgggccctac gaagatgctc agtgctcgag atcgccggga ccggcaccct |
| 481 | gaggaggggg tagttgcaga gctccagggc ttcgcggtgg acaaggcctt cctcacctcc |
| 541 | cacaagggca tcctgctgga aaccgagctg gccctgaccc tcatcatctt catctgcttc |
| 601 | acggcctcca tctctgccta catggccgcg gcgctactgg agttcttcat cacacttgcc |
| 661 | ttcctcttcc tctatgccac ccagtactac cagcgcttcg accgaattaa ctggccctgt |
| 721 | ctggtttttg gcatcatcct ggtttccatc tttgcctatg atgccttcaa gatctaccgg |
| 781 | actgagatgg cacccggggc cagccagggg gaccagcagt gactctgggg ctacctggct |
| 841 | cctaggccca gccagccaga gaggacagtg gagcccagac acgtctcctt gggattcact |
| 901 | agcccccagc ccgccaaacc ccaccccagc cctacacagc agtctggcct gagacgtcac |
| 961 | tggggactta tctgtggagc ctggtgctcc aggatgtggc ttctcatgaa gctctggcca |
| 1021 | gaggagggga acttattggg ggaggggggg tggaggggag gaatctggac ctctaagtca |
| 1081 | ttcccaaatt aaaatattca aattctaaaa aaaaaaaaaa a |

LCN2 (SEQ ID NO: 57) Homo sapiens lipocalin 2 (LCN2), mRNA
NM_005564.4

| | |
|---|---|
| 1 | agggccaccc aggtgagcct ctcactcgcc acctcctctt ccacccctgc caggcccagc |
| 61 | agccaccaca gcgcctgctt cctcggccct gaaatcatgc ccctaggtct cctgtggctg |
| 121 | ggcctagccc tgttgggggc tctgcatgcc caggcccagg actccacctc agacctgatc |
| 181 | ccagccccac ctctgagcaa ggtccctctg cagcagaact tccaggacaa ccaattccag |
| 241 | gggaagtggt atgtggtagg cctggcaggg aatgcaattc tcagagaaga caaagacccg |
| 301 | caaaagatgt atgccaccat ctatgagctg aaagaagaca agagctacaa tgtcacctcc |
| 361 | gtcctgttta ggaaaaagaa gtgtgactac tggatcagga cttttgttcc aggttgccag |
| 421 | cccggcgagt tcacgctggg caacattaag agttaccctg gattaacgag ttacctcgtc |
| 481 | cgagtggtga gcaccaacta caaccagcat gctatggtgt tcttcaagaa agtttctcaa |
| 541 | aacagggagt acttcaagat caccctctac gggagaacca aggagctgac ttcggaacta |
| 601 | aaggagaact tcatccgctt ctccaaatct ctgggcctcc ctgaaaacca catcgtcttc |
| 661 | cctgtcccaa tcgaccagtg tatcgacggc tgagtgcaca ggtgccgcca gctgccgcac |
| 721 | cagcccgaac accattgagg gagctgggag accctcccca cagtgccacc catgcagctg |
| 781 | ctccccaggc caccccgctg atggagcccc accttgtctg ctaaataaac atgtgccctc |
| 841 | aggccaaaaa aaaaaaaaaa aaa |

NLRC4 (SEQ ID NO: 58) NLR family CARD domain containing 4
XM_011533008.1

| | |
|---|---|
| 1 | agaacaagaa ggtatctggt ctacaagaac tcgaggcctc actgaaacgg aaagcaaata |
| 61 | caaagaaact ttattttaaa aacatgtctt ggtctcccaa gaagagggca attggattgc |
| 121 | tcagccagaa tgaagagtag ttttacagaa aaaagaggac aatattggga tcacctttga |
| 181 | cctttccatt tggaaataat attttctatt gtgttataga aaggtgggaa gctttcatcc |
| 241 | agaacaatga atttcataaa ggacaatagc cgagccctta ttcaaagaat gggaatgact |
| 301 | gttataaagc aaatcacaga tgacctattt gtatggaatg ttctgaatcg cgaagaagta |
| 361 | aacatcattt gctgcgagaa ggtggagcag gatgctgcta gagggatcat tcacatgatt |
| 421 | ttgaaaaagg gttcagagtc ctgtaacctc tttcttaaat cccttaagga gtggaactat |
| 481 | cctctatttc aggacttgaa tggacaaagt ctttttcatc agacatcaga aggagacttg |
| 541 | gacgatttgg ctcaggattt aaaggacttg taccataccc catcttttct gaacttttat |
| 601 | ccccttggtg aagatattga cattattttt aacttgaaaa gcaccttcac agaacctgtc |
| 661 | ctgtggagga aggaccaaca ccatcaccgc gtggagcagc tgaccctgaa tggcctcctg |
| 721 | caggctcttc agagcccctg catcattgaa ggggaatctg gcaaaggcaa gtccactctg |
| 781 | ctgcagcgaa ttgccatgct ctggggctcc ggaaagtgca aggctctgac caagttcaaa |
| 841 | ttcgtcttct tcctccgtct cagcagggcc cagggtggac tttttgaaac cctctgtgat |
| 901 | caactcctgg atatacctgg cacaatcagg aagcagacat tcatggccat gctgctgaag |
| 961 | ctgcggcaga gggttctttt ccttcttgat ggctacaatg aattcaagcc ccagaactgc |
| 1021 | ccagaaatcg aagccctgat aaaggaaaac caccgcttca agaacatggt catcgtcacc |
| 1081 | actaccactg agtgcctgag gcacatacgg cagtttggtg ccctgactgc tgaggtgggg |
| 1141 | gatatgacag aagacagcgc ccaggctctc atccgagaag tgctgatcaa ggagcttgct |
| 1201 | gaaggcttgt tgctccaaat tcagaaatcc aggtgcttga ggaatctcat gaagacccct |
| 1261 | ctctttgtgg tcatcacttg tgcaatccag atgggtgaaa gtgagttcca ctctcacaca |
| 1321 | caaacaacgc tgttccatac cttctatgat ctgttgatac agaaaaacaa acacaaacat |
| 1381 | aaaggtgtgg ctgcaagtga cttcattcgg agcctggacc actgtggaga cctagctctg |
| 1441 | gagggtgtgt tctcccacaa gtttgatttc gaactgcagg atgtgtccag cgtgaatgag |
| 1501 | gatgtcctgc tgacaactgg gctcctctgt aaatatacag ctcaaaggtt caagccaaag |
| 1561 | tataaattct ttcacaagtc attccaggag tacacagcag gacgaagact cagcagttta |
| 1621 | ttgacgtctc atgagccaga ggaggtgacc aaggggaatg gttacttgca gaaaatggtt |
| 1681 | tccatttcgg acattacatc cacttatagc agcctgctcc ggtacacctg tgggtcatct |
| 1741 | gtggaagcca ccagggctgt tatgaagcac ctcgcagcag tgtatcaaca cggctgcctt |
| 1801 | ctcggacttt ccatcgccaa gaggcctctc tggagacagg aatctttgca aagtgtgaaa |
| 1861 | aacaccactg agcaagaaat tctgaaagcc ataaacatca attcctttgt agagtgtggc |
| 1921 | atccatttat atcaagagag tacatccaaa tcagccctga gccaagaatt tgaagctttc |
| 1981 | tttcaaggta aaagcttata tatcaactca gggaacatcc ccgattactt atttgacttc |
| 2041 | tttgaacatt tgcccaattg tgcaagtgcc ctggacttca ttaaactgga cttttatggg |
| 2101 | ggagctatgg cttcatggga aaaggctgca gaagacacag gtggaatcca catggaagag |
| 2161 | gccccagaaa cctacattcc cagcagggct gtatctttgt tcttcaactg gaagcaggaa |
| 2221 | ttcaggactc tggaggtcac actccgggat ttcagcaagt tgaataagca agatatcaga |
| 2281 | tatctgggga aaatattcag ctctgccaca agcctcaggc tgcaaataaa gagatgtgct |
| 2341 | ggtgtggctg gaagcctcag tttggtcctc agcacctgta agaacattta ttctctcatg |
| 2401 | gtggaagcca gtcccctcac catagaagat gagaggcaca tcacatctgt aacaaacctg |
| 2461 | aaaaccttga gtattcatga cctacagaat caacggctgc cgggtattgt ggtctgactg |
| 2521 | acagcttggg taacttgaag aaccttacaa agctcataat ggataacata aagatgaatg |
| 2581 | aagaagatgc tataa |

PPBP (SEQ ID NO: 59) - Homo sapiens pro-platelet basic protein
(PPBP), mRNA - NM_002704

| | |
|---|---|
| 1 | acttatctgc agacttgtag gcagcaactc accctcactc agaggtcttc tggttctgga |
| 61 | aacaactcta gctcagcctt ctccaccatg agcctcagac ttgataccac cccttcctgt |
| 121 | aacagtgcga gaccacttca tgccttgcag gtgctgctgc ttctgtcatt gctgctgact |
| 181 | gctctggctt cctccaccaa aggacaaact aagagaaact tggcgaaagg caaagaggaa |
| 241 | agtctagaca gtgacttgta tgctgaactc cgctgcatgt gtataaagac aacctctgga |
| 301 | attcatccca aaaacatcca aagtttggaa gtgatcggga aaggaaccca ttgcaaccaa |
| 361 | gtcgaagtga tagccacact gaaggatggg aggaaaatct gcctggaccc agatgctccc |
| 421 | agaatcaaga aaattgtaca gaaaaaattg gcaggtgatg aatctgctga ttaatttgtt |
| 481 | ctgtttctgc caaacttctt taactcccag gaagggtaga attttgaaac cttgattttc |
| 541 | tagagttctc atttattcag gatacctatt cttactgtat taaaatttgg atatgtgttt |
| 601 | cattctgtct caaaaatcac attttattct gagaaggttg gttaaaagat ggcagaaaga |
| 661 | agatgaaaat aaataagcct ggtttcaacc ctctaattct tgcctaaaca ttggactgta |
| 721 | ctttgcattt ttttctttaa aaatttctat tctaacacaa cttggttgat ttttcctggt |
| 781 | ctactttatg gttattagac atactcatgg gtattattag atttcataat ggtcaatgat |
| 841 | aataggaatt acatggagcc caacagagaa tatttgctca atacattttt gttaatatat |
| 901 | ttaggaactt aatggagtct ctcagtgtct tagtcctagg atgtcttatt taaaatactc |
| 961 | cctgaaagtt tattctgatg tttattttag ccatcaaaca ctaaaataat aaattggtga |
| 1021 | atatgaatct tataaactgt ggttagctgg tttaaagtga atatatttgc cactagtaga |
| 1081 | acaaaaatag atgatgaaaa tgaattaaca tatctacata gttataattc tatcattaga |
| 1141 | atgagcctta taaataagta caatatagga cttcaacctt actagactcc taattctaaa |
| 1201 | ttctactttt ttcatcaaca gaactttcat tcatttttta aaccctaaaa cttataccca |
| 1261 | cactattctt acaaaaatat tcacatgaaa taaaaatttg ctattga |

TREML1 (SEQ ID NO: 60) - Homo sapiens triggering receptor expressed
on myeloid cells like 1 (TREML1), transcript variant 1, mRNA -
NM_178174.3

| | |
|---|---|
| 1 | tcaggcgaat gctgcatcag tgcccaggca agcccaggag ttgacatttc tctgcccagc |
| 61 | catgggcctc accctgctct tgctgctgct cctgggacta gaaggtcagg gcatagttgg |
| 121 | cagcctccct gaggtgctgc aggcacccgt gggaagctcc attctggtgc agtgccacta |
| 181 | caggctccag gatgtcaaag ctcagaaggt gtggtgccgg ttcttgccgg aggggtgcca |
| 241 | gcccctggtg tcctcagctg tggatcgcag agctccagcg ggcaggcgta cgtttctcac |
| 301 | agacctgggt gggggcctgc tgcaggtgga aatggttacc ctgcaggaag aggatgctgg |
| 361 | cgagtatggc tgcatggtgg atggggccag ggggccccag attttgcaca gagtctctct |
| 421 | gaacatactg cccccagagg aagaagaaga gacccataag attggcagtc tggctgagaa |
| 481 | cgcattctca gaccctgcag gcagtgccaa ccctttggaa cccagccagg atgagaagag |
| 541 | catccccttg atctggggtg ctgtgctcct ggtaggtctg ctggtggcag cggtggtgct |
| 601 | gtttgctgtg atggccaaga ggaaacaagg gaacaggctt ggtgtctgtg gccgattcct |
| 661 | gagcagcaga gtttcaggca tgaatccctc ctcagtggtc caccacgtca gtgactctgg |
| 721 | accggctgct gaattgcctt tggatgtacc acacattagg cttgactcac caccttcatt |
| 781 | tgacaatacc acctacacca gcctacctct tgattcccca tcaggaaaac cttcactccc |
| 841 | agctccatcc tcattgcccc ctctacctcc taaggtcctg gtctgctcca agcctgtgac |
| 901 | atatgccaca gtaatcttcc cgggagggaa caagggtgga gggacctcgt gtgggccagc |
| 961 | ccagaatcca cctaacaatc agactccatc cagctaagct gctcatcaca ctttaaactc |
| 1021 | atgaggacca tccctagggg ttctgtgcat ccatccagcc agctcatgcc ctaggatcct |
| 1081 | taggatatct gagcaaccag ggactttaag atctaatcca atgtcctaac tttactaggg |
| 1141 | aaagtgacgc tcagacatga ctgagatgtc ttggggaaga cctccctgca cccaactccc |
| 1201 | ccactggttc ttctaccatt acacactggg ctaaataaac cctaataatg atgtgcaaac |
| 1261 | tcttaatggc tgaatgggaa aggaaactgc ccaagtttga ctaattgctt ggcctgtgaa |
| 1321 | tggaaaagac tctggtctaa aaaaaaaaaa aaaaaa |

PF4 (SEQ ID NO: 61) - Homo sapiens platelet factor 4 (PF4), mRNA -
NM_002619

| | |
|---|---|
| 1 | atcttagttt ccgcaccgca gttcctcggt gtccacttca ggcttccgga ctggaaggac |
| 61 | agccgggaat aaaacgtgcc ggcgaggctc aggagtcatt ggccacagag acccagcccg |
| 121 | agtttcccat cgcactgagc actgagatcc tgctggaagc tctgccgcag catgagctcc |
| 181 | gcagccgggt tctgcgcctc acgccccggg ctgctgttcc tggggttgct gctcctgcca |
| 241 | cttgtggtcg ccttcgccag cgctgaagct gaagaagatg gggacctgca gtgcctgtgt |
| 301 | gtgaagacca cctcccaggt ccgtcccagg cacatcacca gcctggaggt gatcaaggcc |
| 361 | ggaccccact gccccactgc ccaactgata gccacgctga agaatggaag gaaaatttgc |
| 421 | ttggacctgc aagccccgct gtacaagaaa ataattaaga aacttttgga gagttagcta |
| 481 | ctagctgcct acgtgtgtgc atttgctata tagcatactt cttttttcca gtttcaatct |
| 541 | aactgtgaaa gaacttctga tatttgtgtt atccttatga ttttaaataa acaaaataaa |
| 601 | tcaagttgta gtatagtcaa aatacttctt aataatagtg caaaaattgt gttgacacat |
| 661 | aacaatttca tggaagaaaa aaattccggt attttaagca aaaagtattt tgaaggaagg |
| 721 | tgtgaatact ggttatgctt ggtgttacat gttggctgat acatattcat gcatttacat |
| 781 | gattgcagta ctttatagct acatatttac cttgaccatt attattacct ttgccaataa |
| 841 | atatcagtaa cacagaaaaa aaaaaaaaaa aaaaaaaa |

CLEC1B (SEQ ID NO: 62) C-type lectin domain family 1 member B,
transcript variant 1 NM_016509.3

| | |
|---|---|
| 1 | gtccatgtat ctctgagcag ctatgaagaa gcttcctgga aaacaataag caaaggaaaa |
| 61 | caaatgtgtc ccatctcaca tggttctacc ctactaaaga caggaagatc ataaactgac |
| 121 | agatactgaa attgtaaagt tggaaactac attttgcaaa gtcattgaac tctgagctca |
| 181 | gttgcagtac tcgggaagcc atgcaggatg aagatggata catcacctta aatattaaaa |
| 241 | ctcggaaacc agctctcatc tccgttggct ctgcatcctc ctcctggtgg cgtgtgatgg |
| 301 | ctttgattct gctgatcctg tgcgtgggga tggttgtcgg gctggtggct ctggggattt |
| 361 | ggtctgtcat gcagcgcaat tacctacaag gtgagaatga aaatcgcaca ggaactctgc |
| 421 | aacaattagc aaagcgcttc tgtcaatatg tggtaaaaca atcagaacta aagggcactt |
| 481 | tcaaaggtca taaatgcagc ccctgtgaca caaactggag atattatgga gatagctgct |
| 541 | atgggttctt caggcacaac ttaacatggg aagagagtaa gcagtactgc actgacatga |
| 601 | atgctactct cctgaagatt gacaaccgga acattgtgga gtacatcaaa gccaggactc |
| 661 | atttaattcg ttgggtcgga ttatctcgcc agaagtcgaa tgaggtctgg aagtgggagg |
| 721 | atggctcggt tatctcagaa aatatgtttg agtttttgga agatggaaaa ggaaatatga |
| 781 | attgtgctta ttttcataat gggaaaatgc accctacctt ctgtgagaac aaacattatt |
| 841 | taatgtgtga gaggaaggct ggcatgacca aggtggacca actaccttaa tgcaaagagg |
| 901 | tggacaggat aacacagata agggctttat tgtacaataa aagatatgta tgaatgcatc |
| 961 | agtagctgaa aaaaaaaaaa aa |

LCN15 (SEQ ID NO: 63) - Homo sapiens lipocalin 15 (LCN15), mRNA -
NM_203347

| | |
|---|---|
| 1 | caggggctgg agggcagggg aggggatgat gtcattcctg ctcggcgcaa tcctgaccct |
| 61 | gctctgggcg cccacggctc aggctgaggt tctgctgcag cctgacttca atgctgaaaa |
| 121 | gttctcaggc ctctggtacg tggtctccat ggcatctgac tgcagggtct tcctgggcaa |
| 181 | gaaggaccac ctgtccatgt ccaccagggc catcaggccc acagaggagg gcggcctcca |
| 241 | cgtccacatg gagttcccgg gggcggacgg ctgtaaccag gtggatgccg agtacctgaa |
| 301 | ggtgggctcc gagggacact tcagagtccc ggccttgggc tacctggacg tgcgcatcgt |
| 361 | ggacacagac tacagctcct tcgccgtcct ttacatctac aaggagctgg agggggccct |
| 421 | cagcaccatg gtgcagctct acagccggac ccaggatgtg agtccccagg ctctgaagtc |
| 481 | cttccaggac ttctacccga ccctggggct ccccaaggac atgatggtca tgctgcccca |
| 541 | gtcagatgca tgcaaccctg agagcaagga ggcgccctga cacctccgga gccccacccc |
| 601 | cgcccttccc aggtggagcc aaagcagcag gcgcctttgc ccctggagtc aagacccaca |
| 661 | gccctcgggg accacctgga gtctctccat cctccacccc ccgcctgtgg gatgccttgt |
| 721 | gggacgtctc tttctattca ataaacagat gctgcagcct ca |

C1QC (SEQ ID NO: 64) - Homo sapiens complement component 1, q
subcomponent, C chain (C1QC), transcript variant 2, mRNA -
NM_172369

| | |
|---|---|
| 1 | gaccactcag acaccgtgtc ctcttgcctg ggagagggga agcagatctg aggacatctc |
| 61 | tgtgccaggc cagaaaccgc ccacctgcag ttccttctcc gggatggacg tggggcccag |
| 121 | ctccctgccc caccttgggc tgaagctgct gctgctcctg ctgctgctgc ccctcagggg |
| 181 | ccaagccaac acaggctgct acgggatccc agggatgccc ggcctgcccg gggcaccagg |
| 241 | gaaggatggg tacgacggac tgccggggcc caagggggag ccaggaatcc cagccattcc |
| 301 | cgggatccga ggacccaaag ggcagaaggg agaacccggc ttacccggcc atcctgggaa |
| 361 | aaatggcccc atgggacccc ctgggatgcc aggggtgccc ggccccatgg gcatccctgg |
| 421 | agagccaggt gaggagggca gatacaagca gaaattccag tcagtgttca cggtcactcg |
| 481 | gcagacccac cagccccctg cacccaacag cctgatcaga ttcaacgcgg tcctcaccaa |
| 541 | cccgcaggga gattatgaca cgagcactgg caagttcacc tgcaaagtcc ccggcctcta |
| 601 | ctactttgtc taccacgcgt cgcatacagc caacctgtgc gtgctgctgt accgcagcgg |
| 661 | cgtcaaagtg gtcaccttct gtggccacac gtccaaaacc aatcaggtca actcgggcgg |
| 721 | tgtgctgctg aggttgcagg tgggcgagga ggtgtggctg gctgtcaatg actactacga |
| 781 | catggtgggc atccagggct ctgacagcgt cttctccggc ttcctgctct tccccgacta |
| 841 | gggcgggcag atgcgctcga gccccacggg ccttccacct ccctcagctt cctgcatgga |
| 901 | cccaccttac tggccagtct gcatccttgc ctagaccatt ctccccacca gatggacttc |
| 961 | tcctccaggg agcccaccct gacccacccc cactgcaccc cctccccatg ggttctctcc |
| 1021 | ttcctctgaa cttctttagg agtcactgct tgtgtggttc ctgggacact taaccaatgc |
| 1081 | cttctggtac tgccattctt tttttttttt ttttcaagta ttggaagggg tggggagata |
| 1141 | tataaataaa tcatgaaatc aatacataaa aaaaaaaaaa aa |

C1QB (SEQ ID NO: 65) - Homo sapiens complement component 1, q
subcomponent, B chain (C1QB), mRNA - NM_000491

| | |
|---|---|
| 1 | gcccttcccg cctctgggga agggaacttc cgcttcggac cgagggcagt aggctctcgg |
| 61 | ctcctggtcc cactgctgct cagcccagtg gcctcacagg acaccagctt cccaggaggc |
| 121 | gtctgacaca gtatgatgat gaagatccca tggggcagca tcccagtact gatgttgctc |
| 181 | ctgctcctgg gcctaatcga tatctcccag gcccagctca gctgcaccgg gcccccagcc |
| 241 | atccctggca tcccgggtat ccctgggaca cctggccccg atggccaacc tgggacccca |
| 301 | gggataaaag gagagaaagg gcttccaggg ctggctggag accatggtga gttcggagag |
| 361 | aagggagacc cagggattcc tgggaatcca ggaaaagtcg gccccaaggg ccccatgggc |
| 421 | cctaaaggtg gcccaggggc ccctggagcc ccaggcccca aaggtgaatc gggagactac |
| 481 | aaggccaccc agaaaatcgc cttctctgcc acaagaacca tcaacgtccc cctgcgccgg |
| 541 | gaccagacca tccgcttcga ccacgtgatc accaacatga acaacaatta tgagccccgc |
| 601 | agtggcaagt tcacctgcaa ggtgcccggt ctctactact tcacctacca cgccagctct |
| 661 | cgagggaacc tgtgcgtgaa cctcatgcgt ggccgggagc gtgcacagaa ggtggtcacc |
| 721 | ttctgtgact atgcctacaa caccttccag gtcaccaccg gtggcatggt cctcaagctg |
| 781 | gagcaggggg agaacgtctt cctgcaggcc accgacaaga actcactact gggcatggag |
| 841 | ggtgccaaca gcatcttttc cgggttcctg ctctttccag atatggaggc ctgacctgtg |
| 901 | ggctgcttca catccacccc ggctccccct gccagcaacg ctcactctac ccccaacacc |
| 961 | accccttgcc caaccaatgc acacagtagg gcttggtgaa tgctgctgag tgaatgagta |
| 1021 | aataaactct tcaaggccaa ggga |

PCOLCE2 (SEQ ID NO: 66) - Homo sapiens procollagen C-endopeptidase
enhancer 2 (PCOLCE2), mRNA - NM_013363

| | |
|---|---|
| 1 | gaacaaacgg gatattcagc agtggcctgt ggctgccaga gcagctcctc aggggaaact |
| 61 | aagcgtcgag tcagacggca ccataatcgc ctttaaaagt gcctccgccc tgccggccgc |
| 121 | gtatcccccg gctacctggg ccgccccgcg gcggtgcgcg cgtgagaggg agcgcgcggg |
| 181 | cagccgagcg ccggtgtgag ccagcgctgc tgccagtgtg agcggcggtg tgagcgcggt |
| 241 | gggtgcggag gggcgtgtgt gccggcgcgc gcgccgtggg gtgcaaaccc cgagcgtcta |
| 301 | cgctgccatg aggggcgcga acgcctgggc gccactctgc ctgctgctgg ctgccgccac |
| 361 | ccagctctcg cggcagcagt ccccagagag acctgttttc acatgtggtg gcattcttac |
| 421 | tggagagtct ggatttattg gcagtgaagg ttttcctgga gtgtaccctc caaatagcaa |
| 481 | atgtacttgg aaaatcacag ttcccgaagg aaaagtagtc gttctcaatt tccgattcat |
| 541 | agacctcgag agtgacaacc tgtgccgcta tgactttgtg gatgtgtaca atggccatgc |
| 601 | caatggccag cgcattggcc gcttctgtgg cactttccgg cctggagccc ttgtgtccag |
| 661 | tggcaacaag atgatggtgc agatgatttc tgatgccaac acagctggca atggcttcat |
| 721 | ggccatgttc tccgctgctg aaccaaacga aagaggggat cagtattgtg gaggactcct |
| 781 | tgacagacct tccggctctt ttaaaacccc caactggcca gaccgggatt accctgcagg |
| 841 | agtcacttgt gtgtggcaca ttgtagcccc aaagaatcag cttatagaat taaagtttga |
| 901 | gaagtttgat gtggagcgag ataactactg ccgatatgat tatgtggctg tgtttaatgg |
| 961 | cggggaagtc aacgatgcta gaagaattgg aaagtattgt ggtgatagtc cacctgcgcc |
| 1021 | aattgtgtct gagagaaatg aacttcttat tcagttttta tcagacttaa gtttaactgc |
| 1081 | agatgggttt attggtcact acatattcag gccaaaaaaa ctgcctacaa ctacagaaca |
| 1141 | gcctgtcacc accacattcc ctgtaaccac gggtttaaaa cccaccgtgg ccttgtgtca |
| 1201 | acaaaagtgt agacggacgg ggactctgga gggcaattat tgttcaagtg actttgtatt |
| 1261 | agccggcact gttatcacaa ccatcactcg cgatgggagt ttgcacgcca cagtctcgat |
| 1321 | catcaacatc tacaaagagg gaaatttggc gattcagcag gcgggcaaga acatgagtgc |
| 1381 | caggctgact gtcgtctgca agcagtgccc tctcctcaga agaggtctaa attacattat |
| 1441 | tatgggccaa gtaggtgaag atgggcgagg caaaatcatg ccaaacagct ttatcatgat |
| 1501 | gttcaagacc aagaatcaga agctcctgga tgccttaaaa aataagcaat gttaacagtg |
| 1561 | aactgtgtcc atttaagctg tattctgcca ttgcctttga aagatctatg ttctctcagt |
| 1621 | agaaaaaaaa atacttataa aattacatat tctgaaagag gattccgaaa gatgggactg |
| 1681 | gttgactctt cacatgatgg aggtatgagg cctccgagat agctgaggga agttctttgc |
| 1741 | ctgctgtcag aggagcagct atctgattgg aaacctgccg acttagtgcg gtgataggaa |
| 1801 | gctaaaagtg tcaagcgttg acagcttgga agcgtttatt tatacatctc tgtaaaagga |
| 1861 | tattttagaa ttgagttgtg tgaagatgtc aaaaaaagat tttagaagtg caatatttat |
| 1921 | agtgttattt gtttcacctt caagcctttg ccctgaggtg ttacaatctt gtcttgcgtt |
| 1981 | ttctaaatca atgcttaata aaatattttt aaaggattac agaacaacca aaaaaaaaaa |
| 2041 | aaaaaaa |

C1QA (SEQ ID NO: 67) - Homo sapiens component 1, q subcomponent, A
chain (C1QA), mRNA - NM_015991

| | |
|---|---|
| 1 | gccactcctg ctgggcagcc cacagggtcc ctgggcggag ggcaggagca tccagttgga |
| 61 | gttgacaaca ggaggcagag gcatcatgga gggtccccgg ggatggctgg tgctctgtgt |
| 121 | gctggccata tcgctggcct ctatggtgac cgaggacttg tgccgagcac cagacgggaa |
| 181 | gaaaggggag gcaggaagac ctggcagacg ggggcggcca ggcctcaagg gggagcaagg |
| 241 | ggagccgggg gcccctggca tccggacagg catccaaggc cttaaaggag accaggggga |
| 301 | acctgggccc tctggaaacc ccggcaaggt gggctaccca gggcccagcg gccccctcgg |
| 361 | agcccgtggc atcccgggaa ttaaaggcac caagggcagc ccaggaaaca tcaaggacca |
| 421 | gccgaggcca gccttctccg ccattcggcg gaacccccca atggggggca acgtggtcat |
| 481 | cttcgacacg gtcatcacca accaggaaga accgtaccag aaccactccg gccgattcgt |
| 541 | ctgcactgta cccggctact actacttcac cttccaggtg ctgtcccagt gggaaatctg |
| 601 | cctgtccatc gtctcctcct caaggggcca ggtccgacgc tccctgggct tctgtgacac |
| 661 | caccaacaag gggctcttcc aggtggtgtc agggggcatg gtgcttcagc tgcagcaggg |
| 721 | tgaccaggtc tgggttgaaa aagaccccaa aaagggtcac atttaccagg gctctgaggc |
| 781 | cgacagcgtc ttcagcggct tcctcatctt cccatctgcc tgagccaggg aaggaccccc |
| 841 | tcccccaccc acctctctgg cttccatgct ccgcctgtaa aatgggggcg ctattgcttc |
| 901 | agctgctgaa gggagggggc tggctctgag agccccagga ctggctgccc cgtgacacat |
| 961 | gctctaagaa gctcgtttct tagacctctt cctggaataa acatctgtgt ctgtgtctgc |
| 1021 | tgaacatgag cttcagttgc tactcggagc attgagaggg aggcctaaga ataataacaa |
| 1081 | tccagtgctt aagagtca |

TMEM37 (SEQ ID NO: 68) - Homo sapiens transmembrane protein 37
(TMEM37), mRNA - NM_183240

| | |
|---|---|
| 1 | cgatcgaggc tgcagcgcgg ccgccgggcg cagcatgact gccgtcggcg tgcaggccca |
| 61 | gaggcctttg ggccaaaggc agccccgccg gtccttcttt gaatccttca tccggaccct |
| 121 | catcatcacg tgtgtggccc tggctgtggt cctgtcctcg gtctccattt gtgatgggca |
| 181 | ctggctcctg gctgaggacc gcctcttcgg gctctggcac ttctgcacca ccaccaacca |
| 241 | gacgatctgc ttcagagacc tgggccaggc ccatgtgccc gggctggccg tgggcatggg |
| 301 | cctggtacgc agcgtgggcg ccttggccgt ggtggccgcc atttttggcc tggagttcct |
| 361 | catggtgtcc cagttgtgcg aggacaaaca ctcacagtgc aagtgggtca tgggttccat |
| 421 | cctcctcctg gtgtctttcg tcctctcctc cggcgggctc ctgggttttg tgatcctcct |
| 481 | caggaaccaa gtcacactca tcggcttcac cctaatgttt tggtgcgaat tcactgcctc |
| 541 | cttcctcctc ttcctgaacg ccatcagcgg ccttcacatc aacagcatca cccatccctg |
| 601 | ggaatgaccg tggaaatttt aggccccctc cagggacatc agattccaca agaaaatatg |
| 661 | gtcaaaatgg gacttttcca gcatgtggcc tctggtgggg ctgggttgga caagggcctt |
| 721 | gaaacggctg cctgtttgcc gataacttgt gggtggtcag ccagaaatgg cccgggggcc |
| 781 | tctgcacctg gtctgcaggg ccagaggcca ggagggtgcc tcagtgccac caactgcaca |
| 841 | ggcttagcca gatgttgatt ttagaggaag aaaaaaacat tttaaaactc cttcttgaat |
| 901 | tttcttccct ggactggaat acagttggaa gcacaggggt aactggtacc tgagctagct |
| 961 | gcacagccaa ggatagttca tgcctgtttc attgacacgt gctgggatag gggctgcaga |
| 1021 | atccctgggg ctcccagggt tgttaagaat ggatcattct tccagctaag ggtccaatca |
| 1081 | gtgcctagga ctttcttcca ccagctcaaa gggccttcgt atgtatgtcc ctggcttcag |
| 1141 | ctttggtcat gccaaagagg cagagttcag gattccctca gaatgccctg cacacagtag |
| 1201 | gtttccaaac catttgactc ggtttgcctc cctgcccgtt gtttaaacct tacaaaccct |
| 1261 | ggataacccc atcttctagc agctggctgt gcctctggga gctctgccta tcagaaccct |
| 1321 | accttaaggt gggtttcctt ccgagaagag ttcttgagca agctctccca ggagggccca |
| 1381 | cctgactgct aatacacagc cctccccaag gcccgtgtgt gcatgtgtct gtcttttgtg |
| 1441 | agggttagac agcctcaggg caccattttt aatcccagaa cacatttcaa agagcacgta |
| 1501 | tctagacctg ctggactctg cagggggtga gggggaacag cgagagcttg ggtaatgatt |
| 1561 | aacacccatg ctggggatgc atggaggtga agggggccag gaaccagtgg agatttccat |
| 1621 | ccttgccagc acgtctgtac ttctgttcat taaagtgctc cctttctagt cctttttctg |
| 1681 | cccagaa |

TNF (SEQ ID NO: 69) - Homo sapiens tumor necrosis factor (TNF),
mRNA - NM_000594

| | |
|---|---|
| 1 | cagacgctcc ctcagcaagg acagcagagg accagctaag agggagagaa gcaactacag |
| 61 | accccccctg aaaacaaccc tcagacgcca catcccctga caagctgcca ggcaggttct |
| 121 | cttcctctca catactgacc cacggctcca ccctctctcc cctggaaagg acaccatgag |
| 181 | cactgaaagc atgatccggg acgtggagct ggccgaggag gcgctcccca agaagacagg |
| 241 | ggggccccag ggctccaggc ggtgcttgtt cctcagcctc ttctccttcc tgatcgtggc |
| 301 | aggcgccacc acgctcttct gcctgctgca ctttggagtg atcggccccc agagggaaga |
| 361 | gttccccagg gacctctctc taatcagccc tctggcccag gcagtcagat catcttctcg |
| 421 | aaccccgagt gacaagcctg tagcccatgt tgtagcaaac cctcaagctg aggggcagct |
| 481 | ccagtggctg aaccgccggg ccaatgccct cctggccaat ggcgtggagc tgagagataa |
| 541 | ccagctggtg gtgccatcag agggcctgta cctcatctac tcccaggtcc tcttcaaggg |
| 601 | ccaaggctgc ccctccaccc atgtgctcct cacccacacc atcagccgca tcgccgtctc |
| 661 | ctaccagacc aaggtcaacc tcctctctgc catcaagagc ccctgccaga gggagacccc |
| 721 | agagggggct gaggccaagc cctggtatga gcccatctat ctgggagggg tcttccagct |
| 781 | ggagaagggt gaccgactca gcgctgagat caatcggccc gactatctcg actttgccga |
| 841 | gtctgggcag gtctactttg ggatcattgc cctgtgagga ggacgaacat ccaaccttcc |
| 901 | caaacgcctc ccctgcccca atccctttat taccccctcc ttcagacacc ctcaacctct |
| 961 | tctggctcaa aaagagaatt gggggcttag ggtcggaacc caagcttaga actttaagca |
| 1021 | acaagaccac cacttcgaaa cctgggattc aggaatgtgt ggcctgcaca gtgaagtgct |
| 1081 | ggcaaccact aagaattcaa actggggcct ccagaactca ctggggccta cagctttgat |
| 1141 | ccctgacatc tggaatctgg agaccaggga gcctttggtt ctggccagaa tgctgcagga |
| 1201 | cttgagaaga cctcacctag aaattgacac aagtggacct taggccttcc tctctccaga |
| 1261 | tgtttccaga cttccttgag acacggagcc cagccctccc catggagcca gctccctcta |
| 1321 | tttatgtttg cacttgtgat tatttattat ttatttatta tttatttatt tacagatgaa |
| 1381 | tgtatttatt tgggagaccg gggtatcctg ggggacccaa tgtaggagct gccttggctc |
| 1441 | agacatgttt tccgtgaaaa cggagctgaa caataggctg ttcccatgta gccccctggc |
| 1501 | ctctgtgcct tcttttgatt atgtttttta aaatatttat ctgattaagt tgtctaaaca |
| 1561 | atgctgattt ggtgaccaac tgtcactcat tgctgagcct ctgctcccca ggggagttgt |
| 1621 | gtctgtaatc gccctactat tcagtggcga gaaataaagt ttgcttagaa aagaaaaaaa |
| 1681 | aaaaaa |

SLC39A8 (SEQ ID NO: 70) - Homo sapiens solute carrier family 39
member 8 (SLC39A8), transcript variant 1, mRNA - NM_022154

| | |
|---|---|
| 1 | ggcggagaga ataaagcaga aagacaggaa gaggaggtgg agttctacag ttagtgtggt |
| 61 | tttagttttt cctaagaagt ggcgtggttt ggggctttat atccgggagg agcatatgta |
| 121 | cgcaaatcct ggggcgtttg caaacccgga tccggggcgt ctggccccat gcccggccgg |
| 181 | gcgtttgagg gctactgcca cgcagcgttt ctggagcctg ccggctggtg ccctggtggc |
| 241 | ctttatctct gtcccccttt gtcctcttta tctcaggctc tccaggaggc cggggggccc |
| 301 | actccgccta tcgctcccct cggctacgct gccacttcaa tgccccgcag gtcgcgagct |
| 361 | gctgttcttt cgaaggcgtc ggagaaccag gggcgtcccg cgccacctct gactcggagc |
| 421 | agcgccgagc actgacgctc ccgcccttgg gcaaggacgc cagtgcgccc gcgcgcgtcc |
| 481 | ctctgcgcgg cagcccgtcg cgggccctca aggggaagcc caggccagga tggccccggg |
| 541 | tcgcgcggtg gccgggctcc tgttgctggc ggccgccggc ctcggaggag tggcggaggg |
| 601 | gccagggcta gccttcagcg aggatgtgct gagcgtgttc ggcgcgaatc tgagcctgtc |
| 661 | ggcggcgcag ctccagcact tgctggagca gatgggagcc gcctcccgcg tgggcgtccc |
| 721 | ggagcctggc cagctgcact tcaaccagtg tttaactgct gaagagatct tttcccttca |
| 781 | tggcttttca aatgctaccc aaataaccag ctccaaattc tctgtcatct gtccagcagt |
| 841 | cttacagcaa ttgaactttc acccatgtga ggatcggccc aagcacaaaa caagaccaag |
| 901 | tcattcagaa gtttggggat atggattcct gtcagtgacg attattaatc tggcatctct |
| 961 | cctcggattg attttgactc cactgataaa gaaatcttat ttcccaaaga ttttgacctt |
| 1021 | ttttgtgggg ctggctattg ggactctttt ttcaaatgca attttccaac ttattccaga |
| 1081 | ggcatttgga tttgatccca aagtcgacag ttatgttgag aaggcagttg ctgtgtttgg |
| 1141 | tggattttac ctacttttct tttttgaaag aatgctaaag atgttattaa agacatatgg |
| 1201 | tcagaatggt catacccact ttggaaatga taactttggt cctcaagaaa aaactcatca |
| 1261 | acctaaagca ttacctgcca tcaatggtgt gacatgctat gcaaatcctg ctgtcacaga |
| 1321 | agctaatgga catatccatt ttgataatgt cagtgtggta tctctacagg atggaaaaaa |
| 1381 | agagccaagt tcatgtacct gtttgaaggg gcccaaactg tcagaaatag ggacgattgc |
| 1441 | ctggatgata acgctctgcg atgccctcca caatttcatc gatggcctgg cgattggggc |
| 1501 | ttcctgcacc ttgtctctcc ttcagggact cagtacttcc atagcaatcc tatgtgagga |
| 1561 | gtttccccac gagttaggag actttgtgat cctactcaat gcagggatga gcactcgaca |
| 1621 | agccttgcta ttcaacttcc tttctgcatg ttcctgctat gttgggctag cttttggcat |
| 1681 | tttggtgggc aacaatttcg ctccaaatat tatatttgca cttgctggag gcatgttcct |
| 1741 | ctatatttct ctggcagata tgtttccaga gatgaatgat atgctgagag aaaaggtaac |
| 1801 | tggaagaaaa accgatttca ccttcttcat gattcagaat gctggaatgt taactggatt |
| 1861 | cacagccatt ctactcatta ccttgtatgc aggagaaatc gaattggagt aatagaaaat |
| 1921 | ggaagatggt gttgttaata aaggcattta atagataaaa acatctccaa aaaggatttt |
| 1981 | gaagctgatc ctatttagtt aaaaagataa ttttgctttc aactgtaggt ccagaaaact |
| 2041 | aattattggc atcagtctgt gaaatagtcc attatttgtt gttaaaaatg cttcaaaagg |
| 2101 | ttttcagtgt cagtctgaga tgcctggtat ataggagcct ttgggaaata cctatttttc |
| 2161 | agtattccat gcatattaga tatcaccatg aagcaagaga catgcattct ataatcatgt |
| 2221 | agacactcag actcagggga aaatacaagt tatatcctga aagcctttaa aactctatgg |
| 2281 | taggatcaaa gattcaaatg gtttcagaga ggttttattt caattaattt gttctagtgc |
| 2341 | tttcaagagc aagtacatca aaatgtagaa ggtaaaatgt atgcaacact aatataaatt |
| 2401 | attccaagtc tttaaggagc caaagaaaaa aaagatttct cacagctttt tgttctgttt |
| 2461 | tgtatttcaa ttaggaactt gcagtattat tttgaaaacc attctaaaat aataggagtt |
| 2521 | aggaaataaa taaagttttg ctagccctgc taagttcagg cttagaggct tatcgctaag |
| 2581 | tataaacttc accagattcc acgaaaagct ggatagcttt ttttctgact tatgttgtgg |
| 2641 | ttgcacccct cacaaatggc agaacagtat gtaaagctgg taacacctcg gtttcagtgc |
| 2701 | accatgtgtt tgctttgtga aggtgaagaa tatgttggtt tagagaaaga aattggatgt |
| 2761 | aattttatgc aatttacttt taaagacaaa cataactatt tagcagagaa tattttaata |
| 2821 | aatgcaaaac aacagctgga ctgctgtaca tcaaggacag attaactgga aaacatatgt |
| 2881 | tccttatgtg tgatcgagag ccattcagaa aagacttcct ttgtgttcag cctatacttt |
| 2941 | tccatatggt ataccttgaa aaaaattagc acaccatggt tatttttcta ccttttataa |
| 3001 | aagacagagc ctgtttactc atttagaaga tagagaaaat tggtctaaaa ttgaacatcc |
| 3061 | tagattcaca ctcccaagtc acttaaggtg atttgatggt gaggaaaatg attgacaaag |
| 3121 | cccaacaatg atctcaggaa ttacattttc caacagacca aaaaatgttt tcatgtagca |
| 3181 | gcaatgcaga tttggtgaat atttaatata tattttagta tgtatttcac tttatgactg |
| 3241 | acaattaaaa aatattgttt ggccaaatag taaacaccct tttgaaacca tgaaaaaaaa |
| 3301 | aaaaaaaaa |

SLC39A8 (SEQ ID NO: 71) - Homo sapiens solute carrier family 39
member 8 (SLC39A8), transcript variant 3, mRNA - NM_001135147

| | |
|---|---|
| 1 | gactccggaa gagccctttt ctgcagctcc ttggggactg cacgtctcac ttctaagttt |
| 61 | gcggcggaga gaataaagca gaaagacagg aagaggaggt ggagttctac agctctccag |
| 121 | gaggccgggg ggcccactcc gcctatcgct cccctcggct acgctgccac ttcaatgccc |
| 181 | cgcaggtcgc gagctgctgt tctttcgaag gcgtcggaga accaggggcg tcccgcgcca |
| 241 | cctctgactc ggagcagcgc cgagcactga cgctcccgcc cttgggcaag gacgccagtg |
| 301 | cgcccgcgcg cgtccctctg cgcggcagcc cgtcgcgggc cctcaagggg aagcccaggc |
| 361 | caggatggcc ccgggtcgcg cggtggccgg gctcctgttg ctggcggccg ccggcctcgg |
| 421 | aggagtggcg gaggggccag ggctagcctt cagcgaggat gtgctgagcg tgttcggcgc |
| 481 | gaatctgagc ctgtcggcgg cgcagctcca gcacttgctg gagcagatgg gagccgcctc |
| 541 | ccgcgtgggc gtcccggagc ctggccagct gcacttcaac cagtgtttaa ctgctgaaga |
| 601 | gatcttttcc cttcatggct tttcaaatgc tacccaaata accagctcca aattctctgt |
| 661 | catctgtcca gcagtcttac agcaattgaa ctttcaccca tgtgaggatc ggcccaagca |
| 721 | caaaacaaga ccaagtcatt cagaagtttg gggatatgga ttcctgtcag tgacgattat |
| 781 | taatctggca tctctcctcg gattgatttt gactccactg ataaagaaat cttatttccc |
| 841 | aaagattttg accttttttg tggggctggc tattgggact cttttttcaa atgcaatttt |
| 901 | ccaacttatt ccagaggcat ttggatttga tcccaaagtc gacagttatg ttgagaaggc |
| 961 | agttgctgtg tttggtggat tttacctact tttctttttt gaaagaatgc taaagatgtt |
| 1021 | attaaagaca tatggtcaga atggtcatac ccactttgga aatgataact ttggtcctca |
| 1081 | agaaaaaact catcaaccta aagcattacc tgccatcaat ggtgtgacat gctatgcaaa |
| 1141 | tcctgctgtc acagaagcta atggacatat ccattttgat aatgtcagtg tggtatctct |
| 1201 | acaggatgga aaaaaagagc caagttcatg tacctgtttg aaggggccca aactgtcaga |
| 1261 | aatagggacg attgcctgga tgataacgct ctgcgatgcc ctccacaatt tcatcgatgg |
| 1321 | cctggcgatt ggggcttcct gcaccttgtc tctccttcag ggactcagta cttccatagc |
| 1381 | aatcctatgt gaggagtttc cccacgagtt aggagacttt gtgatcctac tcaatgcagg |
| 1441 | gatgagcact cgacaagcct tgctattcaa cttcctttct gcatgttcct gctatgttgg |
| 1501 | gctagctttt ggcattttgg tgggcaacaa tttcgctcca aatattatat ttgcacttgc |
| 1561 | tggaggcatg ttcctctata tttctctggc agatatgttt ccagagatga atgatatgct |
| 1621 | gagagaaaag ataatcaaat gggctactga tgacatcaaa tctcaacttc atttactttg |
| 1681 | gatatatacg gcaaggtagt aatttcatta tctggagtat ttcacctcca catctctgca |
| 1741 | ataccagcac cttcaacagt gattggttag aggttggcat atgacatagt tatgaccatt |
| 1801 | gagacgtgat gctgtgtgtg ctgtgaagtt tctcaaaagg agtaaggaag atacagcctc |
| 1861 | ttctttctag ggactttgtt tgtaggtgtg acacctggag cagccatttt gctactgttt |
| 1921 | tgtttctaat tgtgttcccc gccgccctcc ccgctcgccc cgaaaaacga catgtttgag |
| 1981 | ttctaatccc cagtatttca gaatgtgacc ttatttggaa atagagtcat tgcaaatgta |
| 2041 | atttgttaag atgagctcat actggagtag agtgggttcc tgatctagta tgactggtat |
| 2101 | tcttattaaa aagagaaatt tgaatactag ctacaccatg taaagatgaa ggcagagatc |
| 2161 | gtggtgatgc atctacaatg tcaacaattg ccagcaaatc atcagaatct gggagagagg |
| 2221 | tatggaatga attctctctc gcagccctaa gaaggaacca atcctgtgca cacctgaatc |
| 2281 | ttggacttcc agcctccaca actgtgagac aatacatttt tgttatttaa gctacccagt |
| 2341 | tgtggcactt tattactgca gttctagccg actaatacag ctaccttgag aggaaatagc |
| 2401 | atggggatga aggtgatgtg ctgaagaagc acagcagaaa gtggaaagat ggtccatgag |
| 2461 | gacatcctta gccactgaat tgacaagcct cttccctgga gtccctcttc cttgggattt |
| 2521 | tttttggtgt gtgtaataat cacttttcct aatggtgcaa accattgtga gttgctcttt |
| 2581 | gactgctagt aattaaagca aactaaccaa gtcagccctc cccagtgttc ctttttgcaa |
| 2641 | tctaaattct gatgtccaac tcctccctgg gccactccct gtccccagat gtctctttgt |
| 2701 | atatctaaag aagaaaaaga aggataaatc ctttttctcc caagagatct ccctgctttt |
| 2761 | gtgaaaagca caaagaaggg cattatctaa cttttctcat cattagaagt caagcctggc |
| 2821 | cgtgactccc gttatgacat tttgatggcc acattttatg agaaaagccc aaacagacag |
| 2881 | acctgtctgt aaattctttc atcctttgca ttctttcttt gactgtacct tagagagtgt |
| 2941 | ttgattattg caggggagat ggaaaagaag caggttctgg cctatgctct agttcctcac |
| 3001 | tttctggagt tatcttttgg gcctggagtc tcttggagtg aggaaaggaa gccattttta |
| 3061 | tactttactt atggtagagt taaacttcgt actcttagga ggctacatgc aatctccaag |
| 3121 | aggaagagag tgaagaggtt aagaattatg ccatcaaaat gtgctgctct ggcatattga |
| 3181 | ctatttgaat tacagatatt ttaaaaacat cagatatgaa gaggtcactc tgatcttaat |
| 3241 | tctgtgtctt aacagcagga gatgaaattc ccacatggga gatggcctcc ctgtcacaga |
| 3301 | aggaatacca ttctccctat caatgatgag aaattggaac tgagagaatt ctgtatagac |
| 3361 | cttgttcaag taattcttct attttaaacc tccccacata atttagctgc cttttcaaaa |
| 3421 | ctactgtcct ttgtgcaacc agtatagaag cgattgactc tcactgcatc cttgggtctt |
| 3481 | cttttcccta tatgggctct tttcccatat gggctcttat gtcctgtaaa acatgtatta |
| 3541 | aataaatttg tatgcttttc tcccgttaac ctgtcttaca tcaatttaat tcttgggccc |
| 3601 | aggtaggacc ctaagaggac agaggtcgag ttttgccacc tctgcaagag aaaagagaga |
| 3661 | tgcccagacc accaggcctg ggaaatcttt gatttcccag gctttgaagt gggcactgtg |
| 3721 | tctgaacctg gccctctgaa acttcctgtt cctggacatc ccagctgcta atgtgagttg |
| 3781 | cccttgcact ggctcagggc tgggacatta aaggaatttc ccttttgttg acataacagt |
| 3841 | aggatggcta tagctaacaa taatatatta tttagtttca agtagctaga aggaacatat |
| 3901 | gaatgttccc aacacaaaga aatgacaact atttgagatg atggatatgc taataacctt |
| 3961 | aatctgattc ctatacatta tatgcatcac atatacatac attatatgta taacatcact |
| 4021 | atgtacccca taaatatgtg caattattgt caattaaaca aataaattta aacaaacatt |
| 4081 | aaaaaaaaaa aaaaaaaa |

MRAS (SEQ ID NO: 72) - Homo sapiens muscle RAS oncogene homolog
(MRAS), transcript variant 1, mRNA - NM_012219

| | |
|---|---|
| 1 | agtagcgcaa tctcggctca ctacaacctc tgcctcccgg gttcacgtga ttctcctgcc |
| 61 | tcagcctccc gagtagatga gatcacaggc acgtgccgcc atgccgggtt gacttttgta |
| 121 | tttttagtag agacggggtt tcaccatgtt gcctaggctg gtcttgaact cctgacctca |
| 181 | ggcgattcac ccgcctcggc ctcccaaagt gttaggatta caggcgtgag ccaccgcgcc |
| 241 | cggcttgaat tgtacacttc aaaaggtgga attttatggt gttgaattat atctttattt |
| 301 | ttttaacggg gggaaaatga cgccgctgga gaggagttag cggaactgaa acaatgaaat |
| 361 | ggtgcgcgag tgtcgcctgt ccccgtcgca tccatcccaa cgaagtttgg gccctggaac |
| 421 | ggtgcaccca gaaggcctgc ggggagagac gctggggcat gatctggaag aaagacgtct |
| 481 | caggattcga agggaatgca gctaaggtgg cggcggaggt tcgcctagga ctggggaggc |
| 541 | gtccctaggc tcagaagttg gcccggccgg agcggagatt taaaggttgg agcgcagagg |
| 601 | ctcttaaaga ggccgagtcg aattcccact cggcgtccac cttaaagcca gctccccggc |
| 661 | accacggatc tgacccgggt ctgacctacg agaaacatgg caaccagcgc cgtccccagt |
| 721 | gacaacctcc ccacatacaa gctggtggtg gtgggggatg ggggtgtggg caaaagtgcc |
| 781 | ctcaccatcc agtttttcca gaagatcttt gtgcctgact atgaccccac cattgaagac |
| 841 | tcctacctga aacatacgga gattgacaat caatgggcca tcttggacgt tctggacaca |
| 901 | gctgggcagg aggaattcag cgccatgcgg gagcaataca tgcgcacggg ggatggcttc |
| 961 | ctcatcgtct actccgtcac tgacaaggcc agctttgagc acgtggaccg cttccaccag |
| 1021 | cttatcctgc gcgtcaaaga cagggagtca ttcccgatga tcctcgtggc caacaaggtc |
| 1081 | gatttgatgc acttgaggaa gatcaccagg gagcaaggaa aagaaatggc gaccaaacac |
| 1141 | aatattccgt acatagaaac cagtgccaag gacccacctc tcaatgtcga caaagccttc |
| 1201 | catgacctcg ttagagtaat taggcaacag attccggaaa aaagccagaa gaagaagaag |
| 1261 | aaaaccaaat ggcggggaga ccgggccaca ggcacccaca aactgcaatg tgtgatcttg |
| 1321 | tgacaggcct gaggccctgg gcacagtgac ggtggcctgg ccagccctcg ggacccctcc |
| 1381 | ccacctaact gcactgaaac catttctaac cacaaccctt ggcccaagga cttggtacag |
| 1441 | gaagggagaa gggcaggtgg gcagggagca gacagggtct ggctttgccc agagggcacg |
| 1501 | ggctttccca cctctcaaag agacaaggaa gccacctgta agcagaagca gcatccaagt |
| 1561 | gcccctggcc cccccatgtg ttgattcaac ccggttcctc cccctctctc ggtgggtgtg |
| 1621 | ttgtttattg taactacata gtgttggttt gatgtggaag tgtttatcca catacaaagt |
| 1681 | acaaaacaag ccatgaacaa gcttctttcc cttacccccc atccacaatg tctgagcttg |
| 1741 | gatgtctttt atagattttt aaattatttt agtgattatt attttattaa aggggtctgg |
| 1801 | gctcactgcc tggtgaagtt tcaagtgttc agcagacctc tctggtaaca tatctggaat |
| 1861 | attgttgttg ttttttaacc gagttttccc atcagtgcca aaactcaact caatctgaaa |
| 1921 | gtagagtgtc tgagaggaca gaaggtaatg ggaactgtag ctggaggcct caggccatgg |
| 1981 | gtcaaacctg ggagggaaag agaccctaca catggcctag aaatgagaga agagagaggt |
| 2041 | atttacccag aggattttcc tatggttggg gatgcaaata ttagaaaaca gattgtattt |
| 2101 | tgctgagggg agtggctgtc atgagcatgt cagttctaaa aggggttttc attatcctgg |
| 2161 | aaatgtataa actaaagtaa gctgattggc tttgcaaaca tgttcatttg tttttcagac |
| 2221 | agtatgggtt aagttctctg ccctccccag gggtctgagg aggctctggg tttctcagat |
| 2281 | ctgtctcttg ctgcgttttc acatcagctg tgctgcttgg tgcctctctg atacgaatac |
| 2341 | actgacacgt caaagtaacc taatgtggac accatccaga aaactccagt tcatgctgga |
| 2401 | tcttaaccaa aaatgattca atactgttat cactaaaaca gcaccaagac ctgaagccat |
| 2461 | cttcccttgg agtcaactga ctaccacctc tataagccta gtcaatgagc agaccccttc |
| 2521 | cagtatttgt aaaagtagta ctaggttgcc tttttggcaa tttttattga cctgttgaat |
| 2581 | cttgactata aaatgatctg agaagtaagg aaggctgggc tgatgtgtgg ctctcatata |
| 2641 | ccttctgcaa gggggcagtc tccccagctc cctgatgatg ctcacccccg cccccccacc |
| 2701 | tcaggtgctg ctggtgtgag ccaaagactg gagtttttcc agctggggtg ggagtggaga |
| 2761 | gacaacagga acaacgctgc accaaagaaa aggtcagaat aaaaggcagc acagctggtg |
| 2821 | accttatttt ctagatgtta caaatcaggt cactatgcaa actagaatat cctcagcagg |
| 2881 | tggcctggcc actctggaga aagaaaccca aggaaagtga gcacccaact ggatgccaag |
| 2941 | acacccgggt tctgaaaatg tgctgtgttc ctacctcggc aagatcacca gcactgaggg |
| 3001 | gcccagctgg agaatgattc tgctacaaaa ggagacagtt gagacttttg cttgttggaa |
| 3061 | atcaaacttc ttatttgtct aaattgcccc tttttctgtt cctaaaagga aggataagag |
| 3121 | agaacattcc aggtgaggca cttcaaagtt tccttagacc ctatagtgtt aagaggtatt |
| 3181 | ttaaacacta aaaggacaaa gctcttccca atccttatgc ttccctaagt ggtatctgca |
| 3241 | gcagtttgtt gtgtgcagtt tgatggcagc tgcaaactgg aggtgaggcg gaggaaaggc |
| 3301 | aggtaggaag gagtaaggat ggagatgctc agaatcaaga gcatggcgga gtaggagaag |
| 3361 | aagccctgca cacagggcag tgtccacagc cagaaaactc ctgctgggca ccaaccacta |
| 3421 | cgagcatacc ccatgcccac cgtggagctg caactcctcg acagcactga gtttgatagt |
| 3481 | ctcactggaa gcagatcagc tgatgtagaa cagagacctc ggccataaag gtgagaagac |
| 3541 | atagggattt caaccacaca gttgggacag aagggacagt gcatctgttc atccatcctg |
| 3601 | cacttggccc acgttgaact ccatggtgcc tgagagagac tagttaaggg ttggtcttct |
| 3661 | gtatcctctg ctgttgagcc tctggtaagc tttcatctcc catgaactca tttccccata |
| 3721 | aatgaaatgg gtaaataatg ccccatttgt agaagtgggc cctcatgact gaggtagctt |
| 3781 | ccagataggc cagagtagag tgtagagtgt gccccgtgac atccctccat cttctcctcc |
| 3841 | attatcatct agcagggtca gactgggaaa cctggttggc cacgccacac catgaccgag |
| 3901 | gagccaactg ggacttctgg ctgtttgaca tcctcatgtt cccgttggtc ttccggagaa |
| 3961 | tagtgctacc ctcacatccc ctggagcaca gccttcctga aatgccctca ccccatgcct |
| 4021 | ttgccattgt gtgctctcag atttcttcca ctgtttgaca ccctccttag agggctgctc |
| 4081 | ttttttttcc agagataatc ctagccatcc tctccactcc cacggctggg gacaatggcc |
| 4141 | acttactacc tgtgcacttt gccactcggg acacctggat ggtttctctt aggactttgc |
| 4201 | ccacctcctt ctcatggcac ttgctgtgga aaatgcctgg ctggcctcgt ggggcctgtc |
| 4261 | tcacttttcc aggagacatg acccactaac gtggcaactt taacccaaag gcccctcaga |
| 4321 | catgttacag caaatctgga gccacagaca ggttccctcc attggcagcc cattgtgttt |
| 4381 | gaaattccat gtcgggttta cttggaatga aagatacttg aattattgtg cgcctgtgag |
| 4441 | cgcccagctt ctgtttcata gtcttaacag gtggccattg tcgtgaaacg agtgatgcct |
| 4501 | gaagatctca gtgatgtttg aaccttctgt gtaacttttt attaagtctt tgtatctctc |
| 4561 | gactgattaa taaagaagag aaacacgtaa aaaaaaaaaa aaaaaaaaa |

IFIT1 (SEQ ID NO: 73) - Homo sapiens interferon induced protein
with tetratricopeptide repeats 1 (IFIT1), transcript variant 1,
mRNA - NM_001548

| | |
|---|---|
| 1 | gcaaggacac acccacagct tacaccattg gctgctgttt agctccctta tataacactg |
| 61 | tcttggggtt taaacgtaac tgaaaatcca caagacagaa tagccagatc tcagaggagc |
| 121 | ctggctaagc aaaaccctgc agaacggctg cctaatttac agcaaccatg agtacaaatg |
| 181 | gtgatgatca tcaggtcaag gatagtctgg agcaattgag atgtcacttt acatgggagt |
| 241 | tatccattga tgacgatgaa atgcctgatt tagaaaacag agtcttggat cagattgaat |
| 301 | tcctagacac caaatacagt gtgggaatac acaacctact agcctatgtg aaacacctga |
| 361 | aaggccagaa tgaggaagcc ctgaagagct taaaagaagc tgaaaactta atgcaggaag |
| 421 | aacatgacaa ccaagcaaat gtgaggagtc tggtgacctg gggcaacttt gcctggatgt |
| 481 | attaccacat gggcagactg gcagaagccc agacttacct ggacaaggtg gagaacattt |
| 541 | gcaagaagct ttcaaatccc ttccgctata gaatggagtg tccagaaata gactgtgagg |
| 601 | aaggatgggc cttgctgaag tgtggaggaa aaaattatga acgggccaag gcctgctttg |
| 661 | aaaaggtgct tgaagtggac cctgaaaacc ctgaatccag cgctgggtat gcgatctctg |
| 721 | cctatcgcct ggatggcttt aaattagcca caaaaaatca caagccattt tctttgcttc |
| 781 | ccctaaggca ggctgtccgc ttaaatccag acaatggata tattaaggtt ctccttgccc |
| 841 | tgaagcttca ggatgaagga caggaagctg aaggagaaaa gtacattgaa gaagctctag |
| 901 | ccaacatgtc ctcacagacc tatgtctttc gatatgcagc caagttttac cgaagaaaag |
| 961 | gctctgtgga taaagctctt gagttattaa aaaaggcctt gcaggaaaca cccacttctg |
| 1021 | tcttactgca tcaccagata gggctttgct acaaggcaca aatgatccaa atcaaggagg |
| 1081 | ctacaaaagg gcagcctaga gggcagaaca gagaaaagct agacaaaatg ataagatcag |
| 1141 | ccatatttca ttttgaatct gcagtggaaa aaaagcccac atttgaggtg gctcatctag |
| 1201 | acctggcaag aatgtatata gaagcaggca atcacagaaa agctgaagag aattttcaaa |
| 1261 | aattgttatg catgaaacca gtggtagaag aaacaatgca agacatacat ttccactatg |
| 1321 | gtcggtttca ggaatttcaa aagaaatctg acgtcaatgc aattatccat tatttaaaag |
| 1381 | ctataaaaat agaacaggca tcattaacaa gggataaaag tatcaattct ttgaagaaat |
| 1441 | tggttttaag gaaacttcgg agaaaggcat tagatctgga aagcttgagc ctccttgggt |
| 1501 | tcgtctacaa attggaagga aatatgaatg aagccctgga gtactatgag cgggccctga |
| 1561 | gactggctgc tgactttgag aactctgtga gacaaggtcc ttaggcaccc agatatcagc |
| 1621 | cactttcaca tttcatttca ttttatgcta acatttacta atcatctttt ctgcttactg |
| 1681 | ttttcagaaa cattataatt cactgtaatg atgtaattct tgaataataa atctgacaaa |
| 1741 | atattagttg tgttcaacaa ttagtgaaac agaatgtgtg tatgcatgta agaaagagaa |
| 1801 | atcatttgta tgagtgctat gtagtagaga aaaaatgtta gttaactttg taggaaataa |
| 1861 | aacattggac ttacactaaa tgtttaattc attcatttta ttgtgaaata aaaataaaat |
| 1921 | ccttagctcc tccaccaact gaacagaccc tcttggccaa ggagacccca gaaaccttaa |
| 1981 | aaactaagtt tcccaaccat gacaagatga gagatcattc acacctcatt atattccctc |
| 2041 | ccttgctaac tgccattgga ctttttccac tgagttaaac agaaacccat ggaaaacaaa |
| 2101 | gaacagaaga ctcactcctt ggctgacttc acctagctca ctccacgtag cgccacagcc |
| 2161 | agactcccct cccctcttgc ggtttccaca tgacaactga tcagccttcc ctcctgataa |
| 2221 | gtgaccactg cccacagact ggttctggcc agtccatgga ggctgcacac agggtgcctc |
| 2281 | tatgtccttt gtttcacctt ttgatataga aaggctaatt ttgctgtatt ttaatgttaa |
| 2341 | gtctccacca cagagtgaac acagaatgca tgtgacatac atgtttacat accactattg |
| 2401 | tgtgactgcc cctcatgaat attcatagcc ccccataacc tgttaactat gtgtgtctag |
| 2461 | ccaatccacc aaccataaaa cttctgtaat accctccctt cctccaagag cctgcttttg |
| 2521 | gttgctgtgg taggctctgc ttcccaggct gcaggttgca ggagaggagg ctgcagtggc |
| 2581 | tcacgcctgt aatctcagca cttcgatggg acgaggcagg cagatcacct gaacccagga |
| 2641 | gttcgagagc agccttggca atggcaaaac caaccgtctc tacaaaaaat gcaaaaactt |
| 2701 | agctgggtgt ggtggcatgc acctgtagct tcagttccag ctactcagga ggctgaggtg |
| 2761 | agtggactgc tggagccagg gagttcgagg ctgcagtgtc gagatcttgc cactgcactc |
| 2821 | cattctggat gatagaacga gaccccatct caaaaaaaaa aaaagttctc tccaattgta |
| 2881 | tatagcttgt gattttatgt caacactatc aataaatagc tttcagtgca agaaaccaaa |
| 2941 | aatactgtaa taaacaggca catattcttc ccaaacctca tgcagtttac aatctagtga |
| 3001 | gagacacaga tagcagtaca gagtcaatta aaggttagtt ttcttcatga agatgtttta |
| 3061 | attttaattc aatgtgaaag ggttccaagg agtttatctt gttttatgcc attttatttg |
| 3121 | aagcactact tactaagtca tttgctgata ttaatctagt taaatcaaga aatattacat |
| 3181 | gaaaatgttg ctaaatcaga gatcatgggt aacaatcacc tttgattatg aataatcata |
| 3241 | ttttattgaa aggcaaggca caacaaataa taagaaggaa aaaataaata agcaatgtta |
| 3301 | ttgatctttc attctgtata tgttttgggg ggaatatact agtttctttt agtggctgta |
| 3361 | acaaattacc acaaacttgg tgacttaaaa tttcacagat ttactctttc ttacagttct |
| 3421 | ggaggtcaga agtctgaaat gggtttcaat gagccaaagt caaggtattg atgacgctac |
| 3481 | actcctccgg aggctctagg cagatagcct tttccagctt ccagaggctg cctgaattct |
| 3541 | ttcatccatc ttaaaaacca acagtgtagt agcctcaaat ctctctctct gcttccttct |
| 3601 | tcacatctcc ttctctcctc tgactctttt gcctctttct tctaaggacg caccaggtcc |
| 3661 | acctgcataa tccagaataa ttgccccatc cgcaaatcct taatttaata acatctgcaa |
| 3721 | agtccctttt gctatgtaaa gtagcatgtt cacaggttct ggagacttgg ccatggatac |
| 3781 | gattgcgggg ggggcattat tcttaccaca gagcacccca agaaaatctc caaattttgg |
| 3841 | gcttccaatc cattttgctt caattattta atatttttac tccttccagt agatactgat |
| 3901 | ttcatccatt gcccttaaga aggtaggaca gagattatgg cacatctcac attaaatgct |
| 3961 | atattttcgt tggaaataca ttttttgctt caacttttat tttaaattca agggtacatg |
| 4021 | tgcaggatgt tcaggtttgt tacacaggta aacgtgtgcc atggcggttt gctgaacaga |
| 4081 | tcatcccatc accaacagat catcccattg agaggtgaag ccggctgggc ttctgggttg |
| 4141 | ggtggggact tggagaactt ttctgtctag ctaaagtatt gtaaaatgga ccagtcaaca |
| 4201 | ctctgtaaaa tggaccaatc agctctctgt aaaatggacc aatcagcagg atgtgggtgg |
| 4261 | ggccaagtaa gggaataaaa gcaggccacc cgagctggca gcggcaaccc gctcgggtcc |
| 4321 | ccttccatgc tgtggaagtt ttgttctttc gctctttcaa taaatcttgc tgctgctcaa |
| 4381 | aaaaaaaaaa aaaaaa |

IFI44 (SEQ ID NO: 74) - Homo sapiens interferon induced protein
44 (IFI44), mRNA - NM_006417

| | |
|---|---|
| 1 | tctttgaagc ttcaaggctg ctgaataatt tccttctccc attttgtgcc tgcctagcta |
| 61 | tccagacaga gcagctaccc tcagctctag ctgatactac agacagtaca acagatcaag |
| 121 | aagtatggca gtgacaactc gtttgacatg gttgcacgaa aagatcctgc aaaatcattt |
| 181 | tggagggaag cggcttagcc ttctctataa gggtagtgtc catggattcc gtaatggagt |
| 241 | tttgcttgac agatgttgta atcaagggcc tactctaaca gtgatttata gtgaagatca |
| 301 | tattattgga gcatatgcag aagagagtta ccaggaagga aagtatgctt ccatcatcct |
| 361 | ttttgcactt caagatacta aaatttcaga atggaaacta ggactatgta caccagaaac |
| 421 | actgttttgt tgtgatgtta caaaatataa ctccccaact aatttccaga tagatggaag |
| 481 | aaatagaaaa gtgattatgg acttaaagac aatggaaaat cttggacttg ctcaaaattg |
| 541 | tactatctct attcaggatt atgaagtttt tcgatgcgaa gattcactgg atgaaagaaa |
| 601 | gataaaaggg gtcattgagc tcaggaagag cttactgtct gccttgagaa cttatgaacc |
| 661 | atatggatcc ctggttcaac aaatacgaat tctgctgctg ggtccaattg gagctgggaa |
| 721 | gtccagcttt ttcaactcag tgaggtctgt tttccaaggg catgtaacgc atcaggcttt |
| 781 | ggtgggcact aatacaactg ggatatctga gaagtatagg acatactcta ttagagacgg |
| 841 | gaaagatggc aaatacctgc cgtttattct gtgtgactca ctggggctga gtgagaaaga |
| 901 | aggcggcctg tgcagggatg acatattcta tatcttgaac ggtaacattc gtgatagata |
| 961 | ccagtttaat cccatggaat caatcaaatt aaatcatcat gactacattg attccccatc |
| 1021 | gctgaaggac agaattcatt gtgtggcatt tgtatttgat gccagctcta ttcaatactt |
| 1081 | ctcctctcag atgatagtaa agatcaaaag aattcgaagg gagttggtaa acgctggtgt |
| 1141 | ggtacatgtg gctttgctca ctcatgtgga tagcatggat ttgattacaa aaggtgacct |
| 1201 | tatagaaata gagagatgtg agcctgtgag gtccaagcta gaggaagtcc aaagaaaact |
| 1261 | tggatttgct ctttctgaca tctcggtggt tagcaattat tcctctgagt gggagctgga |
| 1321 | ccctgtaaag gatgttctaa ttctttctgc tctgagacga atgctatggg ctgcagatga |
| 1381 | cttcttagag gatttgcctt ttgagcaaat agggaatcta agggaggaaa ttatcaactg |
| 1441 | tgcacaagga aaaaaataga tatgtgaaag gttcacgtaa atttcctcac atcacagaag |
| 1501 | attaaaattc agaaaggaga aaacacagac caaagagaag tatctaagac caaagggatg |
| 1561 | tgttttatta atgtctagga tgaagaaatg catagaacat tgtagtactt gtaaataact |
| 1621 | agaaataaca tgatttagtc ataattgtga aaaataataa taatttttct tggatttatg |
| 1681 | ttctgtatct gtgaaaaaat aaatttctta taaaactcgg gtctaaaaaa aaaaaaaaaa |
| 1741 | aa |

RPGRIP1 (SEQ ID NO: 75) (retinitis pigmentosa G7Pase regulator
interacting protein 1, NM_020366.3

| | |
|---|---|
| 1 | atgtcacatc tggtggaccc tacatcagga gacttgccag ttagagacat agatgctata |
| 61 | cctctggtgc taccagcctc aaaaggtaag aatatgaaaa ctcaaccacc cttgagcagg |
| 121 | atgaaccggg aggaattgga ggacagtttc tttcgacttc gcgaagatca catgttggtg |
| 181 | aaggagcttt cttggaagca acaggatgag atcaaaaggc tgaggaccac cttgctgcgg |
| 241 | ttgaccgctg ctggccggga cctgcgggtc gcggaggagg cggcgccgct ctcggagacc |
| 301 | gcaaggcgcg ggcagaaggc gggatggcgg cagcgcctct ccatgcacca gcgcccccag |
| 361 | atgcaccgac tgcaagggca tttccactgc gtcggccctg ccagcccccg ccgcgcccag |
| 421 | cctcgcgtcc aagtgggaca cagacagctc cacacagccg gtgcaccggt gccggagaaa |
| 481 | cccaagaggg ggccaaggga caggctgagc tacacagccc ctccatcgtt taaggagcat |
| 541 | gcgacaaatg aaaacagagg tgaagtagcc agtaaaccca gtgaacttgt ttctggttct |
| 601 | aacagcataa tttctttcag cagtgtcata agtatggcta aacccattgg tctatgcatg |
| 661 | cctaacagtg cccacatcat ggccagcaat accatgcaag tggaagagcc acccaagtct |
| 721 | cctgagaaaa tgtggcctaa agatgaaaat tttgaacaga gaagctcatt ggagtgtgct |
| 781 | cagaaggctg cagagcttcg agcttccatt aaagagaagg tagagctgat tcgacttaag |
| 841 | aagctcttac atgaaagaaa tgcttcattg gttatgacaa aagcacaatt aacagaagtt |
| 901 | caagaggcat acgaaacctt gctccagaag aatcagggaa tcctgagtgc agcccatgag |
| 961 | gccctcctca agcaagtgaa tgagctcagg gcagagctga aggaagaaag caagaaggct |
| 1021 | gtgagcttga agagccaact ggaagatgtg tctatcttgc agatgactct gaaggagttt |
| 1081 | caggagagag ttgaagattt ggaaaaagaa cgaaaattgc tgaatgacaa ttatgacaaa |
| 1141 | ctcttagaaa gcatgctgga cagcagtgac agctccagtc agccccactg gagcaacgag |
| 1201 | ctcatagcgg aacagctaca gcagcaagtc tctcagctgc aggatcagct ggatgctgag |
| 1261 | ctggaggaca agagaaaagt tttacttgag ctgtccaggg agaaagccca aaatgaggat |
| 1321 | ctgaagcttg aagtcaccaa catacttcag aagcataaac aggaagtaga gctcctccaa |
| 1381 | aatgcagcca caatttccca acctcctgac aggcaatctg aaccagccac tcacccagct |
| 1441 | gtattgcaag agaacactca gatcgagcca agtgaaccca aaaaccaaga agaaaagaaa |
| 1501 | ctgtcccagg tgctaaatga gttgcaagta tcacacgcag agaccacatt ggaactagaa |
| 1561 | aagaccaggg acatgcttat tctgcagcgc aaaatcaacg tgtgttatca ggaggaactg |
| 1621 | gaggcaatga tgacaaaagc tgacaatgat aatagagatc acaaagaaaa gctggagagg |
| 1681 | ttgactcgac tactagacct caagaataac cgtatcaagc agctggaagg tattttaaga |
| 1741 | agccatgacc ttccaacatc tgaacagctc aaagatgttg cttatggcac ccgaccgttg |
| 1801 | tcgttatgtt tggaaacact gccagcccat ggagatgagg ataaagtgga tatttctctg |
| 1861 | ctgcatcagg gtgagaatct ttttgaactg cacatccacc aggccttcct gacatctgcc |
| 1921 | gccctagctc aggctggaga tacccaacct accactttct gcacctattc cttctatgac |
| 1981 | tttgaaaccc actgtacccc attatctgtg gggccacagc ccctctatga cttcacctcc |
| 2041 | cagtatgtga tggagacaga ttcgcttttc ttacactacc ttcaagaggc ttcagcccgg |
| 2101 | cttgacatac accaggccat ggccagtgaa cacagcactc ttgctgcagg atggatttgc |
| 2161 | tttgacaggg tgctagagac tgtggagaaa gtccatggct tggccacact gattggagct |
| 2221 | ggtggagaag agttcggggt tctagagtac tggatgaggc tgcgtttccc cataaaaccc |
| 2281 | agcctacagg cgtgcaataa acgaaagaaa gcccaggtct acctgtcaac cgatgtgctt |
| 2341 | ggaggccgga aggcccagga agaggagttc agatcggagt cttgggaacc tcagaacgag |
| 2401 | ctgtggattg aaatcaccaa gtgctgtggc ctccggagtc gatggctggg aactcaaccc |
| 2461 | agtccatatg ctgtgtaccg cttcttcacc ttttctgacc atgacactgc catcattcca |
| 2521 | gccagtaaca acccctactt tagagaccag gctcgattcc cagtgcttgt gacctctgac |
| 2581 | ctggaccatt atctgagacg ggaggccttg tctatacatg tttttgatga tgaagactta |
| 2641 | gagcctggct cgtatcttgg ccgagcccga gtgcctttac tgcctcttgc aaaaaatgaa |
| 2701 | tctatcaaag gtgattttaa cctcactgac cctgcagaga aacccaacgg atctattcaa |
| 2761 | gtgcaactgg attggaagtt tccctacata ccccctgaga gcttcctgaa accagaagct |
| 2821 | cagactaagg ggaaggatac caaggacagt tcaaagatct catctgaaga ggaaaaggct |
| 2881 | tcatttcctt cccaggatca gatggcatct cctgaggttc ccattgaagc tggccagtat |
| 2941 | cgatctaaga gaaaacctcc tcatggggga gaaagaaagg agaaggagca ccaggttgtg |
| 3001 | agctactcaa gaagaaaaca tggcaaaaga ataggtgttc aaggaaagaa tagaatggag |
| 3061 | tatcttagcc ttaacatctt aaatggaaat acaccagagc aggtgaatta cactgagtgg |
| 3121 | aagttctcag agactaacag cttcataggt gatggcttta aaaatcagca cgaggaagag |
| 3181 | gaaatgacat tatcccattc agcactgaaa cagaaggaac ctctacatcc tgtaaatgac |
| 3241 | aaagaatcct ctgaacaagg ttctgaagtc agtgaagcac aaactaccga cagtgatgat |
| 3301 | gtcatagtgc cacccatgtc tcagaaatat cctaaggcag attcagagaa gatgtgcatt |
| 3361 | gaaattgtct ccctggcctt ctacccagag gcagaagtga tgtctgatga gaacataaaa |
| 3421 | caggtgtatg tggagtacaa attctacgac ctacccttgt cggagacaga gactccagtg |
| 3481 | tccctaagga agcctagggc aggagaagaa atccactttc actttagcaa ggtaatagac |
| 3541 | ctggacccac aggagcagca aggccgaagg cggtttctgt tcgacatgct gaatggacaa |
| 3601 | gatcctgatc aaggacattt aaagtttaca gtggtaagtg atcctctgga tgaagaaaag |
| 3661 | aaagaatgtg aagaagtggg atatgcatat cttcaactgt ggcagatcct ggagtcagga |
| 3721 | agagatattc tagagcaaga gctagacatt gttagccctg aagatctggc taccccaata |
| 3781 | ggaaggctga aggtttccct tcaagcagct gctgtcctcc atgctattta caaggagatg |
| 3841 | actgaagatt tgttttcatg aaggaacaag tgctattcca atctaaaagt ctctgaggga |
| 3901 | accatagtaa aaagtctctt ataaagttag cttgctataa catgaaaaaa |

DISC1 (SEQ ID NO: 76) - Homo sapiens disrupted in schizophrenia 1
(DISC1), transcript variant q, mRNA - NM_001164554

| | |
|---|---|
| 1 | ggaaggagca ggaggcagcc caggcggagc gggaggagct ggcagcgggg cgcatgccag |
| 61 | gcgggggtcc tcagggcgcc ccagccgccg ccggcggcgg cggcgtgagc caccgcgcag |
| 121 | gcagccggga ttgcttacca cctgcagcgt gctttcggag gcggcggctg gcacggaggc |
| 181 | cgggctacat gagaagctcg acagggcctg ggatcgggtt cctttcccca gcagtgggca |
| 241 | cactgttccg gttcccagga ggggtgtctg gcgaggagtc ccaccactcg gagtccaggg |
| 301 | ccagacagtg tggccttgac tcgagaggcc tcttggtccg gagccctgtt tccaagagtg |
| 361 | cagcagcccc tactgtgacc tctgtgagag gaacctcggc gcactttggg attcagctca |
| 421 | gaggtggcac cagattgcct gacaggctta gctggccgtg tggccctggg agtgctgggt |
| 481 | ggcagcaaga gtttgcagcc atggatagtt ctgagaccct ggacgccagc tgggaggcag |
| 541 | cctgcagcga tggagcaagg cgtgtccggg cagcaggctc tctgccatca gcagagttga |
| 601 | gtagcaacag ctgcagccct ggctgtggcc ctgaggtccc cccaacccct cctggctctc |
| 661 | acagtgcctt tacctcaagc tttagcttta ttcggctctc gcttggctct gccggggaac |
| 721 | gtggagaagc agaaggctgc ccaccatcca gagaggctga gtcccattgc cagagccccc |
| 781 | aggagatggg agccaaagct gccagcttgg acgggcctca cgaggacccg cgatgtctct |
| 841 | ctcggccctt cagtctcttg gctacacggg tctctgcaga cttggcccag gccgcaagga |
| 901 | acagctccag gccagagcgt gacatgcatt ctttaccaga catggaccct ggctcctcca |
| 961 | gttctctgga tccctcactg gctggctgtg gtggtgatgg gagcagcggc tcaggggatg |
| 1021 | cccactcttg ggacaccctg ctcaggaaat gggagccagt gctgcgggac tgcctgctga |
| 1081 | gaaaccggag gcagatggag gtaatatcct taagattaaa acttcagaaa cttcaggaag |
| 1141 | atgcagttga gaatgatgat tatgataaag gtgagtttta atttgtttat tgattgtttt |
| 1201 | gtcatcatgt cccaattttc tttccatctt tactcatatc taccttttga atcccaaaag |
| 1261 | aattgtacaa tctgttcctc tgatcatctc taccagggaa tagttgactc ttttacagca |
| 1321 | ttattgtttt gtagatttca aagtacttca tgaacattaa tcctttgggt tataaatata |
| 1381 | accttatcaa ttgtccagaa acacttagca tactcacaca ataaaaatta tattagcttg |
| 1441 | ccacctgtct gcccatggtg tgatgtatct ataatccact tgttcataaa aaatattcgt |
| 1501 | ctgacaccta tgatatgcta gggaatatgg gagacaatag gaaagtaaga cagacatggt |
| 1561 | atctgccctt gtgatgccag taaacttaag ccttcatggg gctctctgac ttcataattt |
| 1621 | ccagaaccag agataggaaa tgaggtgaat ttgagaaatg tcagactgtg ccaaaggggg |
| 1681 | tcacatgcat caaatttcca catacat |

CXCR1 (SEQ ID NO: 77) C-X-C motif chemokine receptor 1, NM_000634.2

| | |
|---|---|
| 1 | tattcatcaa gtgccctcta gctgttaagt cactctgatc tctgactgca gctcctactg |
| 61 | ttggacacac ctggccggtg cttcagttag atcaaaccat tgctgaaact gaagaggaca |
| 121 | tgtcaaatat tacagatcca cagatgtggg attttgatga tctaaatttc actggcatgc |
| 181 | cacctgcaga tgaagattac agcccctgta tgctagaaac tgagacactc aacaagtatg |
| 241 | ttgtgatcat cgcctatgcc ctagtgttcc tgctgagcct gctgggaaac tccctggtga |
| 301 | tgctggtcat cttatacagc agggtcggcc gctccgtcac tgatgtctac ctgctgaacc |
| 361 | tggccttggc cgacctactc tttgccctga ccttgcccat ctgggccgcc tccaaggtga |
| 421 | atggctggat ttttggcaca ttcctgtgca aggtggtctc actcctgaag gaagtcaact |
| 481 | tctacagtgg catcctgctg ttggcctgca tcagtgtgga ccgttacctg gccattgtcc |
| 541 | atgccacacg cacactgacc cagaagcgtc acttggtcaa gtttgtttgt cttggctgct |
| 601 | ggggactgtc tatgaatctg tccctgccct tcttcctttt ccgccaggct taccatccaa |
| 661 | acaattccag tccagtttgc tatgaggtcc tgggaaatga cacagcaaaa tggcggatgg |
| 721 | tgttgcggat cctgcctcac acctttggct tcatcgtgcc gctgtttgtc atgctgttct |
| 781 | gctatggatt caccctgcgt acactgttta aggcccacat ggggcagaag caccgagcca |
| 841 | tgagggtcat ctttgctgtc gtcctcatct tcctgctttg ctggctgccc tacaacctgg |
| 901 | tcctgctggc agacaccctc atgaggaccc aggtgatcca ggagagctgt gagcgccgca |
| 961 | acaacatcgg ccgggccctg gatgccactg agattctggg atttctccat agctgcctca |
| 1021 | accccatcat ctacgccttc atcggccaaa attttcgcca tggattcctc aagatcctgg |
| 1081 | ctatgcatgg cctggtcagc aaggagttct tggcacgtca tcgtgttacc tcctacactt |
| 1141 | cttcgtctgt caatgtctct tccaacctct gaaaaccatc gatgaaggaa tatctcttct |
| 1201 | cagaaggaaa gaataaccaa caccctgagg ttgtgtgtgg aaggtgatct ggctctggac |
| 1261 | aggcactatc tgggttttgg ggggacgcta taggatgtgg ggaagttagg aactggtgtc |
| 1321 | ttcaggggcc acaccaacct tctgaggagc tgttgaggta cctccaagga ccggcctttg |
| 1381 | cacctccatg gaaacgaagc accatcattc ccgttgaacg tcacatcttt aacccactaa |
| 1441 | ctggctaatt agcatggcca catctgagcc ccgaatctga cattagatga gagaacaggg |
| 1501 | ctgaagctgt gtcctcatga gggctggatg ctctcgttga ccctcacagg agcatctcct |
| 1561 | caactctgag tgttaagcgt tgagccacca agctggtggc tctgtgtgct ctgatccgag |
| 1621 | ctcagggggg tggttttccc atctcaggtg tgttgcagtg tctgctggag acattgaggc |
| 1681 | aggcactgcc aaaacatcaa cctgccagct ggccttgtga ggagctggaa acacatgttc |
| 1741 | cccttggggg tggtggatga acaaagagaa agagggtttg gaagccagat ctatgccaca |
| 1801 | agaaccccct ttacccccat gaccaacatc gcagacacat gtgctggcca cctgctgagc |
| 1861 | cccaagtgga acgagacaag cagcccttag cccttcccct ctgcagcttc caggctggcg |
| 1921 | tgcagcatca gcatccctag aaagccatgt gcagccacca gtccattggg caggcagatg |
| 1981 | ttcctaataa agcttctgtt ccgtgcttgt ccctgtggaa gtatcttggt tgtgacagag |
| 2041 | tcaagggtgt gtgcagcatt gttggctgtt cctgcagtag aatgggggca gcacctccta |
| 2101 | agaaggcacc tctctgggtt gaagggcagt gttccctggg gctttaactc ctgctagaac |
| 2161 | agtctcttga ggcacagaaa ctcctgttca tgcccatacc cctggccaag gaagatccct |
| 2221 | ttgtccacaa gtaaaaggaa atgctcctcc agggagtctc agcttcaccc tgaggtgagc |
| 2281 | atcatcttct gggttaggcc ttgcctaggc atagccctgc ctcaagctat gtgagctcac |
| 2341 | cagtccctcc ccaaatgctt tccatgagtt gcagtttttt cctagtctgt tttccctcct |
| 2401 | tggagacagg gccctgtcgg tttattcact gtatgtcctt ggtgcctgga gcctactaaa |
| 2461 | tgctcaataa ataatgatca caggaaaaaa aaaaaaaaaa aa |

HCAR2 (SEQ ID NO: 78) - Homo sapiens hydroxycarboxylic acid receptor
2 (HCAR2), mRNA - NM_177551

| | |
|---|---|
| 1 | accacacaga cacacacctc cttgctggag cattcactag gcgaggcgct ccatcggact |
| 61 | cactagccgc actcatgaat cggcaccatc tgcaggatca ctttctggaa atagacaaga |
| 121 | agaactgctg tgtgttccga gatgacttca ttgtcaaggt gttgccgccg gtgttggggc |
| 181 | tggagtttat cttcgggctt ctgggcaatg gccttgccct gtggattttc tgtttccacc |
| 241 | tcaagtcctg gaaatccagc cggattttcc tgttcaacct ggcagtggct gactttctac |
| 301 | tgatcatctg cctgcccttc ctgatggaca actatgtgag gcgttgggac tggaagtttg |
| 361 | gggacatccc ttgccggctg atgctcttca tgttggctat gaaccgccag ggcagcatca |
| 421 | tcttcctcac ggtggtggcg gtagacaggt atttccgggt ggtccatccc caccacgccc |
| 481 | tgaacaagat ctccaatcgg acagcagcca tcatctcttg ccttctgtgg ggcatcacta |
| 541 | ttggcctgac agtccacctc ctgaagaaga agatgccgat ccagaatggc ggtgcaaatt |
| 601 | tgtgcagcag cttcagcatc tgccatacct tccagtggca cgaagccatg ttcctcctgg |
| 661 | agttcttcct gcccctgggc atcatcctgt tctgctcagc cagaattatc tggagcctgc |
| 721 | ggcagagaca aatggaccgg catgccaaga tcaagagagc catcaccttc atcatggtgg |
| 781 | tggccatcgt ctttgtcatc tgcttccttc ccagcgtggt tgtgcggatc cgcatcttct |
| 841 | ggctcctgca cacttcgggc acgcagaatt gtgaagtgta ccgctcggtg gacctggcgt |
| 901 | tctttatcac tctcagcttc acctacatga acagcatgct ggaccccgtg gtgtactact |
| 961 | tctccagccc atcctttccc aacttcttct ccactttgat caaccgctgc ctccagagga |
| 1021 | agatgacagg tgagccagat aataaccgca gcacgagcgt cgagctcaca ggggacccca |
| 1081 | acaaaaccag aggcgctcca gaggcgttaa tggccaactc cggtgagcca tggagcccct |
| 1141 | cttatctggg cccaacctct ccttaaataa ccatgccaag aagggacatt gtcaccaaga |
| 1201 | accaggatct ctggagaaac agttgggctg ttgcatcgag taatgtcact ggactcggcc |
| 1261 | taaggtttcc tggaacttcc agattcagag aatgcgattt agggaaacgg tggcagatga |
| 1321 | gtgggagact ggttgcaagg tgtgaccgca ggaatcctgg aggaatagag agtaaagctt |
| 1381 | ctaggcatct gaaacttttg cttcatctct gacgctcgca ggactgaaga tgggcaaatt |
| 1441 | gtaggcattt ctgctgagca gagttggagc cagagatcta cttgtgactt gttggccttc |
| 1501 | ttcccacatc tgcctcagac tggagggggc tcagctcctg gggtgatatc tagcctgctt |
| 1561 | gtgagctcta gcagggataa ggagagctga gattggaggg aattgtgttg ctcctggagg |
| 1621 | gagcccaggc atcattaaac aagccagtag gtcacctggc ttccgtggac caattcatct |
| 1681 | ttcagacaag ctttagcaga aatggactca gggaagagac tcacacgctt tggttaatat |
| 1741 | ctgtgtttcc ggtgggtgta ataggggatt agccccagaa gggactgagc taaacagtgt |
| 1801 | tattatggga aaggaaatgg cattgctgct ttcaaccagc gactaatgca atccattcct |
| 1861 | ctcttgttta tagtaatcta agggttgggc agttaaaacg gcttcaggat agaaagctgt |
| 1921 | ttcccacctc tgtttgcttt taacattaaa agggaaatgt gcctctgccc cacagttaga |
| 1981 | ggggtgcacg ttcctcctgg ttccttcgct tgtgtttctg tacttaccaa aaatctacca |
| 2041 | tttcaataaa ttttgatagg agacaaaaaa aaaaaaaaaa aa |

EPSTI1 (SEQ ID NO: 79) - Homo sapiens epithelial stromal interaction
1 (breast)(EPSTIL1), transcript variant 2, mRNA - NM_033255

| | |
|---|---|
| 1 | aaaaccgctg cctctgcact ttggaatccc atcttgagac tcgctaagcg tcccagccgc |
| 61 | atccctcccg cagcgacggc ggcccgggac ccgcgggctg tgaaccatga acacccgcaa |
| 121 | tagagtggtg aactccgggc tcggcgcctc ccctgcctcc cgcccgaccc gggatcccca |
| 181 | ggacccttct gggcggcaag gggagctgag ccccgtggaa gaccagagag agggtttgga |
| 241 | ggcagcccct aagggccctt cgcgggagag cgtcgtgcac gcgggccaga ggcgcacaag |
| 301 | tgcatacacc ttgatagcac caaatataaa ccggagaaat gagatacaaa gaattgcgga |
| 361 | gcaggagctg gccaacctgg agaagtggaa ggagcagaac agagctaaac cggttcacct |
| 421 | ggtgcccaga cggctaggtg gaagccagtc agaaactgaa gtcagacaga aacaacaact |
| 481 | ccagctgatg caatctaaat acaagcaaaa gctaaaaaga gaagaatctg taagaatcaa |
| 541 | gaaggaagct gaagaagctg aactccaaaa aatgaaggca attcagagag agaagagcaa |
| 601 | taaactggag gagaaaaaaa gacttcaaga aaaccttaga agagaagcat ttagagagca |
| 661 | tcagcaatac aaaaccgctg agttcttgag caaactgaac acagaatcgc cagacagaag |
| 721 | tgcctgtcaa agtgctgttt gtggcccaca atcctcaaca tgggccagaa gctgggctta |
| 781 | cagagattct ctaaaggcag aagaaaacag aaaattgcaa aagatgaagg atgaacaaca |
| 841 | tcaaaagagt gaattactgg aactgaaacg gcagcagcaa gagcaagaaa gagccaaaat |
| 901 | ccaccagact gaacacagga gggtaaataa tgcttttctg gaccgactcc aaggcaaaag |
| 961 | tcaaccaggt ggcctcgagc aatctggagg ctgttggaat atgaatagcg gtaacagctg |
| 1021 | gggtatatga gaaaatattg actcctatct ggccttcatc aactgacctc gaaaagcctc |
| 1081 | atgagatgct ttttcttaat gtgattttgt tcagcctcac tgtttttacc ttaatttcaa |
| 1141 | ctgcccacac acttgaccgt gcagtcagga gtgactggct tctccttgtc ctcatttatg |
| 1201 | catgtttgga ggagctgatt cctgaactca tatttaatct ctactgccag ggaaatgcta |
| 1261 | cattattttt ctaattggaa gtataattag agtgatgttg gtagggtaga aaaagaggga |
| 1321 | gtcacttgat gctttcaggt taatcagagc tatgggtgct acaggcttgt ctttctaagt |
| 1381 | gacatattct tatctaattc tcagatcagg ttttgaaagc tttgggggtc tttttagatt |
| 1441 | ttaatcccta ctttctttat ggtacaaata tgtacaaaag aaaaaggtct tatattcttt |
| 1501 | tacacaaatt tataaataaa ttttgaactc cttctgtata aatgggtcat ttttattttt |
| 1561 | aatgaaaagt tattggggtt ttctctcttg aagggtctca ttttaattcc cttttccagg |
| 1621 | ccgtatagat caaatatagt actgtcatta ctgttggctc ttgttttggt cttgacttac |
| 1681 | taatagtgtt accctgattt tcagaggggg acagtttatc tccagaaagg ccaatgtttg |
| 1741 | tatacacatc agctagacac aaatatagac atcatatgta gtttgtacat gtttcagaaa |
| 1801 | cttgtttttt ctttgctctg tgtaacctat ttcctattgc tagttcagtt ggctttctta |
| 1861 | ttcacttctg tgaccctgaa ccagttctca gaccctagag tgtaagagca ttgattttct |
| 1921 | acgctgtgta atctagctca atccctctgt cccctccgcc tcaccgtccc ccagccacca |
| 1981 | cattgtatag caaaagcatt acattcaatc ctagaataaa ggtaaataca acaaatcatc |
| 2041 | tttgcagctg gacaactaat aatactttgc agcattaaga gatcttctgt gttaccagtc |
| 2101 | actctgttga aatgaacttt ccgaatctct ttattcagga aaacatgggg ttttgaaatt |
| 2161 | cttgggccaa gagacataac tgaggggttc gcagagctag gcaagggtgc actaggaaag |
| 2221 | ggccacattg gtgggtgggg ggtaacagag aacagatggt gtcaggaagt ttctctggag |
| 2281 | taaataatgt ggatattctt ggtttccctc tcctccgcca gctgaagctg tgttagtgct |
| 2341 | gttgacacta atataaaatg tttggtccat ttgaaatcct tgtcattgcc ttatatgggg |
| 2401 | gaaactcaat cccccagcct gtgttggaaa tatcaccaaa ctgattgtaa atgtgcggct |
| 2461 | gtagcagaca ttttagtgtg gtggtgtgca gccatttcgg ccctacacct gccagcctgg |
| 2521 | ctaccttaca gttgtgttcc gatttttgcg tctatgcttg gtgtgcctca cttgctgcat |
| 2581 | tttccagcat gcaaccagga gttgacgtag gaaaaaggga tgctttctta ctttggaagc |
| 2641 | tctcagggaa gttggtgtca atttctcctc cactgcctgg cctaccctgc actcccaaag |
| 2701 | attttgtgca gatgggtagt tccatttttt aaaaattgtg cagatatgga aaattgtgac |
| 2761 | ttacttcatg accagaacta tctagaatat gtgtgggggt ataaacatct tgcttaacca |
| 2821 | aatatctatg taggcagagg taaccaggag agaagcaaga cttgctgcct aaaggagccc |
| 2881 | accattttac ttttcacatt taatctgcca cgttgaatca attggaataa aacctgactc |
| 2941 | gcaggtgact ggacaggaaa tcccaaagtt ccaccatttc tatgcttaat tttaatgtcc |
| 3001 | ccccgctttt ttttttgtag aaaataaaaa caagaaaatc gttccaatgt aagatgtttg |
| 3061 | ttatagaaac tttaggcaat acaggtgtgt aataaaatgt ttaataaact tctaaacact |
| 3121 | tttgtatttg gattta |

LILRB4 (SEQ ID NO: 80) Homo sapiens leukocyte immunoglobulin like
receptor B4 (LILRB4), transcript variant 1, mRNA NM_001278426.3

| | |
|---|---|
| 1 | agaacctggt gcctgcctca gccctagctc tggggaaatg aaagccaggc tggggttcaa |
| 61 | atgagggcag tttcccttcc tgtgggctgc tgatggaaca accccatgac gagaaggacc |
| 121 | cagcctccaa gcggccacac cctgtgtgtc tctttgtcct gccggcactg aggactcatc |
| 181 | catctgcaca gctggggccc ctgggaggag acgccatgat ccccaccttc acggctctgc |
| 241 | tctgcctcgg gctgagtctg ggccccagga cccacatgca ggcagggccc ctccccaaac |
| 301 | ccaccctctg ggctgagcca ggctctgtga tcagctgggg gaactctgtg accatctggt |
| 361 | gtcaggggac cctggaggct cgggagtacc gtctggataa agaggaaagc ccagcaccct |
| 421 | gggacagaca gaacccactg gagcccaaga acaaggccag attctccatc ccatccatga |
| 481 | cagaggacta tgcagggaga taccgctgtt actatcgcag ccctgtaggc tggtcacagc |
| 541 | ccagtgaccc cctggagctg gtgatgacag gagcctacag taaacccacc ctttcagccc |
| 601 | tgccgagtcc tcttgtgacc tcaggaaaga gcgtgaccct gctgtgtcag tcacggagcc |
| 661 | caatggacac ttttcttctg atcaaggagc gggcagccca tcccctactg catctgagat |
| 721 | cagagcacgg agctcagcag caccaggctg aattccccat gagtcctgtg acctcagtgc |
| 781 | acggggggac ctacaggtgc ttcagctcac acggcttctc ccactacctg ctgtcacacc |
| 841 | ccagtgaccc cctggagctc atagtctcag gatccttgga gggtcccagg ccctcaccca |
| 901 | caaggtccgt ctcaacagct gcaggccctg aggaccagcc cctcatgcct acagggtcag |
| 961 | tcccccacag tggtctgaga aggcactggg aggtactgat cggggtcttg gtggtctcca |
| 1021 | tcctgcttct ctccctcctc ctcttcctcc tcctccaaca ctggcgtcag ggaaaacaca |
| 1081 | ggacattggc ccagagacag gctgatttcc aacgtcctcc aggggctgcc gagccagagc |
| 1141 | ccaaggacgg gggcctacag aggaggtcca gcccagctgc tgacgtccag ggagaaaact |
| 1201 | tctgtgctgc cgtgaagaac acacagcctg aggacggggt ggaaatggac actcggcaga |
| 1261 | gcccacacga tgaagacccc caggcagtga cgtatgccaa ggtgaaacac tccagaccta |
| 1321 | ggagagaaat ggcctctcct ccctccccac tgtctgggga attcctggac acaaaggaca |
| 1381 | gacaggcaga agaggacaga cagatggaca ctgaggctgc tgcatctgaa gccccccagg |
| 1441 | atgtgaccta cgcccggctg cacagcttta ccctcagaca gaaggcaact gagcctcctc |
| 1501 | catcccagga aggggcctct ccagctgagc ccagtgtcta tgccactctg gccatccact |
| 1561 | aatccagggg ggacccagac cccacaagcc atggagactc aggaccccag aaggcatgga |
| 1621 | agctgcctcc agtagacatc actgaacccc agccagccca gacccctgac acagaccact |
| 1681 | agaagattcc gggaacgttg ggagtcacct gattctgcaa agataaataa tatccctgca |
| 1741 | ttatcaaaat aaagtagcag acctctcaat tcacaatgag ttaactgata aaacaaaaca |
| 1801 | gaagtcagac aatgttttaa attgaatgat catgtaaata ttacacatca aaccaatgac |
| 1861 | atgggaaaat gggagcttct aatgaggaca aacaaaaaat agagaaaaat taataaagtc |
| 1921 | aaaatgttta ttcttgaaaa cattaatgat acatgaatct tggccacaat gagaaaaata |
| 1981 | aaaatgaaaa aagagcaggc atccatttcc atacaggaac aaaataggag gcagcactac |
| 2041 | agaccctaca cacagcttta cagaggtgaa agaaaactgt cagcaattct atgctgacat |
| 2101 | aacagaaaat gtagatgaga tagatgaaat acgaaaaatt acagtttact taatgaacat |
| 2161 | aaggataaat agaaaaactg aatcatcata cataaacata tataaaatgc attgatcctg |
| 2221 | taatcaaaaa tgttcccaca aagtaaatgc cacttcagca aggtttgttg gtggtttttt |
| 2281 | caaactctta tgcactcatg aaacacacag acacacacac acacaaactt gcataaattt |
| 2341 | tccctgagaa tattttgtat atatttacac aaatacattt gatcagacta ggaacaagtt |
| 2401 | gataccaaaa cctgaaaagg aaactacaga atgggaaagt catagaagat ctctcacaga |
| 2461 | aatataaatc ccttaacaaa tattaacaag taagattcat gtctctataa aatagacagt |
| 2521 | atatcatgac cacactggtt ttttgttatc ctttgatttt gtttatgaaa agcaaggata |
| 2581 | gcttaatttt caaaaactca atcaatgtaa ttcagtattt taacaaaagg aatgaaaaat |
| 2641 | tatcatctca atagacaaag cttttgtctg agcacctttt catatagctg ctgaccattt |
| 2701 | gtatgtcttc ttttgagaaa tgcctgttca gctactttgc ccatgtttca agtagttttt |
| 2761 | ggtttcttgc tgttgctttg ttttagttcc ttacatattt ttgcatatta accctttatc |
| 2821 | aggtatacag cttgcaacta ttttctccca tttctgagtt gtctcttcat tctgtttgca |
| 2881 | gaagctgttt agaagccaca ccttttgtct atttttgctt ttgttgcttg tgttttcagg |
| 2941 | gccatatcca aaaaaacctt gcccggacca acgtcttgaa gcttttctcc cacccatttt |
| 3001 | tgtatatggg ataagggttc aatttcattc ttcttcatat gaatatcccc aggatgtgtc |
| 3061 | ctatgcccag ctgcacagct taccctcaaa cagaaaataa tgaagccttc ttcctcccag |
| 3121 | gaaaggggac gttcagctga gccgagtgtg tatactgctc tggccatcca ctagcccagg |
| 3181 | gaggacccag acctccacac tccatggaga ctcagttctc ctaggaccat ttattcaaaa |
| 3241 | ggactgccct ctcttgttct tggaaacttt gttgaggatc aattcaccat aaatatgtgt |
| 3301 | gtttccttct ttgctttcat ccctgttgca ctgatcactg tacctgtttc tattccagtt |
| 3361 | ccatgatgtc ttcctggctg tagctttgta ggatatttgg ggattccata gtgtgatatc |
| 3421 | cccttcttcc ctttgctcaa gattgttttg gctatttggg gtccttttgt agtcccattc |
| 3481 | aaattttagg attgtttttc tatttctgtg gaaaacgacc ttggaatttt gttaggaatt |
| 3541 | gcattgagtc tgcaggtatg aacttttttt taaagttcca gggcacatgt acaggacctg |
| 3601 | cagctttgtt acataggtag gcttgtgcca tggtggtttg ctgcacctat caacccatta |
| 3661 | cctagttatt aagcccagca tgcattagct ctttttcctg atgctctccc tcccttcatc |
| 3721 | atccgccctc ccactacaag ccccagtgtg tgttgttccc ctccctgtgt ccatgtgttc |
| 3781 | tcattgttat acgaacattt taacaatgtt aattcttgca gaccatgaac ataagctacc |
| 3841 | ttcccattta tatgcgtctt gttcaatttc attcatcaat gttataaaga ttttagtgca |
| 3901 | ga |

LILRB5 (SEQ ID NO: 81) Homo sapiens leukocyte immunoglobulin like
receptor B5 (LILRB5), transcript variant 2, mRNA NM_006840.4

| | |
|---|---|
| 1 | cagtcttgtg acagggagaa cccagcctcc agtccacact ctgcgtgttt ttgtgtcctg |
| 61 | ccaggcaccg tggtctcatc cgcctgcaca gctgagtcca gtgggagctg acgccatgac |
| 121 | cctcaccctc tcagtcctga tttgcctcgg gctgagtgtg ggccccagga cctgcgtgca |
| 181 | ggcaggcacc ctccccaaac ccaccctctg ggctgagcca gcctctgtga tagctcgggg |
| 241 | gaagcccgtg accctctggt gtcaggggcc cctggagact gaggagtacc gtctggataa |
| 301 | ggagggactc ccatgggccc ggaagagaca gaacccactg gagcctggag ccaaggccaa |
| 361 | gttccacatt ccatccacgg tgtatgacag tgcagggcga taccgctgct actatgagac |
| 421 | ccctgcaggc tggtcagagc ccagtgaccc cctggagctg gtggcgacag gattctatgc |
| 481 | agaacccact cttttagccc tgccgagtcc tgtggtggcc tcaggaggaa atgtgaccct |
| 541 | ccagtgtgat acactggacg gacttctcac gtttgttctt gttgaggaag aacagaagct |
| 601 | ccccaggacc ctgtactcac agaagctccc caaagggcca tcccaggccc tgttccctgt |
| 661 | gggtcccgtg acccccagct gcaggtggag gttcagatgc tattactatt acaggaaaaa |
| 721 | ccctcaggtg tggtcgaacc ccagtgacct cctggagatt ctggtcccag gcgtgtctag |
| 781 | gaagccctcc ctcctgatcc cgcagggctc tgtcgtggcc cgcggaggca gcctgaccct |
| 841 | gcagtgtcgc tctgatgtcg gctatgacat attcgttctg tacaaggagg gggaacatga |
| 901 | cctcgtccag ggctctggcc agcagcccca ggctgggctc tcccaggcca acttcaccct |
| 961 | gggccctgtg agccgctccc acgggggcca gtacagatgc tacggtgcac acaacctctc |
| 1021 | ccctaggtgg tcggccccca gcgaccccct ggacatcctg atcgcaggac tgatccctga |
| 1081 | catacccgcc ctctcggtgc agccgggccc caaggtggcc tcaggagaga acgtgaccct |
| 1141 | gctgtgtcag tcatggcatc agatagacac tttctttttg accaaggagg gggcagccca |
| 1201 | tcccccgctg tgtctaaagt caaagtacca gtcttataga caccaggctg aattctccat |
| 1261 | gagtcctgtg acctcagccc agggtggaac ctaccgatgc tacagcgcaa tcaggtccta |
| 1321 | cccctacctg ctgtccagcc ctagttaccc ccaggagctc gtggtctcag gaccctctgg |
| 1381 | ggatcccagc ctctcaccta caggctccac ccccacacct ggccctgagg accagcccct |
| 1441 | cacccccacg gggttggatc cccagagtgg tctgggaagg cacctggggg ttgtgactgg |
| 1501 | ggtctcagtg gccttcgtcc tgctgctgtt cctcctcctc ttcctcctcc tccgacatcg |
| 1561 | gcatcagagc aaacacagga catcggccca tttctaccgt cctgcagggg ctgcggggcc |
| 1621 | agagcccaag gaccagggcc tgcagaagag ggccagccca gttgctgaca tccaggagga |
| 1681 | aattctcaat gctgccgtga aggacacaca gcccaaggac ggggtggaga tggatgctcg |
| 1741 | ggctgctgca tctgaagccc cccaggatgt gacctacgcc cagctgcaca gcttgaccct |
| 1801 | cagacgggag gcaactgagc ctcctccatc ccaggaaagg gaacctccag ctgaacccag |
| 1861 | catctacgcc cccctggcca tccactagcc cacgggggac ccagatctca tactcaacag |
| 1921 | aaggagactc agagactcca gaaggcacag gagctgcccc cagtggacac caatgaaccc |
| 1981 | cagccagcct ggacccctaa caaagaccac caggacatcc tgggaactct gggactcact |
| 2041 | agattctgca gtcaaagatg actaatatcc ttgcattttt gaaatgaagc cacagacttc |
| 2101 | tcaataaatc aatgagctga gaaaactgaa acagaaatta gagcatggta taaatttgga |
| 2161 | atgataatgt aaatattaca cattaaatga tgaaatcgga aaactacaaa tgagcgaatg |
| 2221 | aattagaaaa gaataaaacc tacgtaatta atgaccttgg caatgacag |

NECAB1 (SEQ ID NO: 82) - Homo sapiens N-terminal EF-hand calcium
binding protein 1 (NECAB1), mRNA - NM_022351

| | |
|---|---|
| 1 | agaggccgga ggagggaggg ggggacaccg agcgcggaga gcgcggagag cgcggaggga |
| 61 | ggcgcgcgcg ggagcgaaca ccctcccgga tccagagccc ggcggcggcg aagcagcagc |
| 121 | tgcggccgcg cccttgccag agccggtgcg tccgcctagc cccgctccgc ctgaggccgt |
| 181 | cagggctccc gaggatggaa gattcccagg agacatcgcc gtcctccaac aactcctcgg |
| 241 | aggagctcag ctctgctctg cacctgtcca agggcatgtc gatcttcctc gacatactga |
| 301 | ggagagcaga caaaaatgat gatggaaaat tatcctttga agaattcaaa gcatattttg |
| 361 | cagatggtgt tctcagtgga gaagaattac acgagctttt ccataccatt gatacacata |
| 421 | atactaataa tcttgacaca gaagagctat gtgaatattt ttctcagcac ttgggcgagt |
| 481 | atgagaatgt actagcagca cttgaagacc tgaatctttc catcctgaag gcaatgggca |
| 541 | aaacaaagaa agactaccaa gaagcctcca atttggaaca attcgtaact agatttttat |
| 601 | tgaaggaaac cctgaatcag ctgcagtctc tccagaattc cctggaatgt gccatggaaa |
| 661 | ctactgagga gcaaacccgt caagaaaggc aagggccagc caagccagaa gtcctgtcga |
| 721 | ttcaatggcc tggaaaacga tcaagccgcc gagtccagag acacaacagc ttctccccaa |
| 781 | acagccctca gtttaatgtc agcggtccag gcttattaga agaagacaac cagtggatga |
| 841 | cccagataaa tagactccag aaattaattg atagactgga aaagaaggat ctcaaactcg |
| 901 | aaccaccaga agaagaaatt attgaaggga atactaaatc tcacatcatg cttgtgcagc |
| 961 | ggcagatgtc tgtgatagaa gaggacctgg aagaattcca gctcgctctg aaacactacg |
| 1021 | tggagagtgc ttcctcccaa agtggatgct tgcgtatttc tatacagaag ctttcaaatg |
| 1081 | aatctcgcta catgatctat gagttctggg agaatagtag tgtatggaat agccaccttc |
| 1141 | agacaaatta tagcaagaca ttccaaagaa gtaatgtgga tttcttggaa actccagaac |
| 1201 | tcacatctac aatgctagtt cctgcttcgt ggtggatcct gaacaactag atgttcctag |
| 1261 | acattttctt tatggttcca agtgcaaaac aggtgttctt atctaaaacg tcaattagaa |
| 1321 | aattatctgc ggttgttaat ctactgtata tttttgtttg gtatatttac taagtgcact |
| 1381 | ctttcaaaac ttattctata actttatcaa ttcatgtgaa ttttagctca attttcaaag |
| 1441 | ttcactaata ttctcaatat ttaatgctaa atgctttgct acattgtaac tcacctaaaa |
| 1501 | ccttttagtg acaaaatcct aatatgtgga aaaaagcata tgcataaagg aataatattg |
| 1561 | tgaaaatgaa tctgttatga taaagaaaaa ataaagtgga aacttttaga gtattacttc |
| 1621 | atagggcaga ttttgtaaac tgtcgtatac tgtaaagggt taaatcagcg ttttgtgatt |
| 1681 | tttaagtaac tgtgagtgaa gtttattctt caacaatgtc tactccatcc ccaacccaac |
| 1741 | tcacagccct atgactacta tctttgcatt agttaaaaag ttagtatata ggcatcaaac |
| 1801 | aaccttggct gtaacctata gaatctctat ccacgtatca ggttatagac tggtttttca |
| 1861 | aaagtgaaca atcctgtgat aagttggagt accatttagt aatacagcaa cattgtgtca |
| 1921 | tttattagca tcataattct ttgttatgta agttaaatat atcaagaaag aagagactgt |
| 1981 | ttggaaaaat gtggttcaag ttttatgcta tatagttttg gtatgcgata cagacagcta |
| 2041 | acttttctta tgaaaaatac atatttgcat gtaaacaatg atttcaaaat acttgaaaaa |
| 2101 | taaaatttta acccaaatga ataactaaga aatataaaac aagcacaaaa tcttagggaa |
| 2161 | gtcataaaat agtagtgaaa gtattagaca gaagacatct gttttcgaat ttcaacacta |
| 2221 | gaatgactaa aactatctac ctatagaact atctgtagat agtatactat ctacactctg |
| 2281 | ctcaacaagc tcagaaatta aatattttta ataataaaaa tctgttctgg ttataaacct |
| 2341 | tgctaatgaa aatacaatac atataaaaat gtatagccat gttattttct agtataaatt |
| 2401 | cctttgaaac tataagtctt tgaggaaaat tataaggtaa aattttcctg tttttccccc |
| 2461 | tttgaaaaac tcaggaaaaa aggaagattg aactaataaa attttatttc ttaaatataa |
| 2521 | atttgaccta aaatattttc tcaaactaat tcatgaaaca gcaactttta ccaatacctt |
| 2581 | tgtatactct cagttctcat tcagtataaa taaaatttta aaatcctttc atagttctat |
| 2641 | tagaaataag tagtaaattt tgatatattg tacatacaca cgtgtgtgtg tgtgtgtgtg |
| 2701 | tgtgtgtgtg tgtgtgtgtg tgtatttgtg tgcctctggt caactctaag gatgacagac |
| 2761 | actgtgtaac aacacctggg tcaactcttt taatttatat acaaagcaaa gaacaacatt |
| 2821 | aatggagatg cacaatgatt attcaaacaa gctatatata tgtacaaagg caaacagaca |
| 2881 | cataacagtc tctgcagact gattgtatat agtaagaaaa gatcaaaaga ctttaaaacc |
| 2941 | taaatgactt ttgacataca aactcttctt gagaatgttt gttgtaaatg gtttcaaaaa |
| 3001 | tacaaattat agccaatcaa aacattgctt tggttggtgc atttaagtat ccaactcaaa |
| 3061 | aagcatatca aatattttgg gtactaggca gtttccaaag tagcatggta gtattacttg |
| 3121 | ttaaaagggt tctgttttca ttaacagtac taagtggaag ggatctgcag attccaaact |
| 3181 | ggaataagct ctatcatatt ctgaaacaag aattagaatg acttgagaac gggcaaataa |
| 3241 | caaagcaaac caatataatt atatggtcat tctgacccca gctcttatac aaattataca |
| 3301 | tgtatttttg tgtatgtttg tgagagttgt atgtatgtga atgtgtgtga gtgtgtattc |
| 3361 | acatacacat atatactgga acctatagta gaaaaggaaa ctagtagggc caaaaaaaaa |
| 3421 | aagaaaaaga aaaagaaaaa agaaaaaaaa agaaaaaact gggacctaag tataaatatc |
| 3481 | tcatcctaaa gtaaacaata agtttatagt taacgaagat ttttttctat ttaaaacccc |
| 3541 | attttcctaa agaacaaagt agtaaaataa aaaaaaattt aaaaaattaa aaaataaaaa |
| 3601 | aaagaggtca ctaaaagacc aagatgggaa acgtaacatg gaatacaagc ttgaatctgg |
| 3661 | gaaacaagct caaaaaacag tgaaaaaaaa agtactggtt taggagttca aaattcagtg |
| 3721 | aaacaaactt tttgccaata gacctaggga tctaagaaat agaattaggg agagtttaca |
| 3781 | cttacttacc tatatatcag cagatttttc tggaagaaac cctttttttt ttttttttta |
| 3841 | aacagtaaca tccagagtga caaattgcag gagatttaat gtataaaatt tctaggaatt |
| 3901 | aaaagcttaa acccaaaaat tgttcatcca tatataagaa attatcgaat taaaacttaa |
| 3961 | tgtatgtcaa ttattttcaa atgtctaaat tcctttggaa ataaaaatat cagttttact |
| 4021 | ttgaaattgc tcaacttctg ctattcatat ggtctgatta gtgacataag tagcagccgt |
| 4081 | ttttcaactt cagtttcatt cactaccatt gtttccaaat tatcaatctc tgctagtaat |
| 4141 | tatgaattta agaccatatt attatcaaaa acctagagac caatcatata tctgaaaaga |
| 4201 | aatacccatt aaaaattctg cctcctgttt attgagaact atgcattgaa cattctgaat |
| 4261 | cattctaagc ctctggagag actgtaatga tccattcatg agctggtatg aaatcagtgg |
| 4321 | gaagcagaac aacagaagga actttaaaag caacaagcaa ttatgtacca tatatacact |
| 4381 | gtagcaaata ttttatattt gagagtcttt acagcttaca tttccattcc attattacaa |
| 4441 | gtgatgaaaa acaaaaaatt gcaagtagta tgcaaattat aaatatacag tcttcccact |
| 4501 | tcactaacca aattcctact ttccagtgtt acttcccaat ttatgcagga aacctcctgc |
| 4561 | aaagctgaaa ctgattagaa aattctttat attttaaaat agctctttct catttttaga |
| 4621 | gaagtcaaat agccaaccat caaaattaag aataaattga attgtcacag tccattacag |
| 4681 | ttattgttgc tagatccacc tcatttgcag atgtccaaac ttaaattcat ctgttcttaa |
| 4741 | aatgctactt aaaactttgg ttgttttcct gtaatataaa agaaaaagtt aatttatcaa |
| 4801 | ttgattgaat acagttttta ctaattagtt tatcaaacca aatactgtga acgtaccagg |
| 4861 | tgtttacaga tttaaatgca tgttaccata gaaactatta aagtaactag aactgtcaaa |
| 4921 | taacaaaacg gctcatgttt ttaaaatata tgtaactcat tttaaaatat attaaattgt |
| 4981 | attccaaacc tgttcttctg tttctgtggc acctaggttt aaaatatgta ttaatgtgta |
| 5041 | aatcacaagt aaaatgaatt ctaatgtaca agtttgtttt aaaaagtgta tgtcaagctt |
| 5101 | ttatttacac aataaaatgt tattaaagat ggaaagtcta |

NECAB2 (SEQ ID NO: 83) - Homo sapiens N-terminal EF-hand calcium
binding protein 2 (NECAB2), mRNA - NM_019065

| | |
|---|---|
| 1 | gcggcgggcg cggcgcgatg tgcgagcggg cggcgcgcct gtgcagggcc ggcgcgcaca |
| 61 | ggctgctccg ggagccgccg cagcagggcc gggcgctggg cgggctgctg cgctgggtgg |
| 121 | gcgccaggat gggcgagccc cgggagtcgc tggcccccgc cgcccccgcg gaccccggcc |
| 181 | cagcctcgcc gcgcgggggc accgccgtca tcctggacat tttccgccgt gcggacaaaa |
| 241 | atgatgatgg gaagctgtcc ttggaggaat tccagctctt ctttgcagat ggcgtcctta |
| 301 | atgagaaaga actggaggat ctctttcaca cgattgactc tgacaacacc aaccatgtgg |
| 361 | acaccaagga gctgtgtgat tactttgtgg accacatggg tgactatgag gatgtcctgg |
| 421 | cctccctgga gaccttgaat cactctgtcc tgaaggccat gggttatacc aagaaggtat |
| 481 | atgagggtgg gagcaacgtg gaccagtttg tgacccgctt cctcctgaag gagacggcca |
| 541 | atcagatcca gtcgctgctg agctcagtgg agagtgcggt ggaggccatc gaggaacaga |
| 601 | ccagccagct ccgacagaac cacatcaaac ccagccacag cgcggcacag acctggtgtg |
| 661 | gaagccccac tcccgcctct gcccccaacc acaagctcat ggctatggaa caaggcaaga |
| 721 | cccttccatc tgccacggag gatgcaaagg aagagggtct ggaagcccag atcagccgct |
| 781 | tggcagagct gattgggagg ctggagagca aagcactgtg gttcgacctg cagcagcgcc |
| 841 | tgtcagatga agatggcacc aacatgcacc tgcagctggt ccggcaggag atggccgtgt |
| 901 | gccccgagca actgagcgag tttctggact ctctgcgcca gtatctgcgg gggaccactg |
| 961 | gcgtgaggaa ctgcttccac atcactgccg tgaggctctc agatggcttc acctttgtca |
| 1021 | tctatgagtt ctgggagaca gaggaggcgt ggaagaggca cctgcagagc cccctgtgta |
| 1081 | aggcgttccg gcacgtcaag gtggacacac tgagccagcc tgaggccctc tccaggatct |
| 1141 | tggtgccagc tgcttggtgc acggtgggac gggactgaca gcctcccaga ggcccgtgga |
| 1201 | ggagcccacc agccccttct tcttgtgaag gaaatcccgt ttttttctag acagacactt |
| 1261 | tggtgcagaa gcttcttttc aatccatcct ccacaagaag gtgtttccct gttgttaagt |
| 1321 | gaaggaggcc gcccctgccc ccacctgaga aggcagagca gtgtctgtgc tgccaggtcc |
| 1381 | tggtgaagcc caaggttgaa gggggcggct tcctggagcc agcacccctg cctcctggtc |
| 1441 | ctggcctctc ccctacccct cacatggcca cgcatgaccc acactgacca caccctgccc |
| 1501 | tcttcggtga cattcttcta cctagtagga gtcatgcccc tgtagtgccc aacctagcca |
| 1561 | ggtagccacc actgtgccca ggcgccaaat aaaccctggt tgggaaaaaa aaaaaaaaaa |
| 1621 | aaaaaaaaaa aaaaaa |

PKHD1 (SEQ ID NO: 84) - Homo sapiens polycystic kidney and hepatic
disease 1 (autosomal Recessive)(PKHD1), transcript variant 1, mRNA -
NM_138694

| | |
|---|---|
| 1 | acatttggct gggacacaaa cgagttaggt gagttatagt ctaacgtgag tgagttacct |
| 61 | gcgtggtggc tgcaggctga gctctaacca gataacatgt ccacggattc ttgacagaga |
| 121 | gaaataagat cacttagaaa gaaaggatca tttctccctt gagtcacaag gagacagaaa |
| 181 | cagaaaaaaa agcacaaaag ctatgctgct ccaatcaaaa ctgaaaatgc ttttaatgtc |
| 241 | tgagcaatct taagagtatt gatttaagtg gacagaatga ctgcctggct gatctctctg |
| 301 | atgagtattg aagtactact tttggcagta cgtcacctga gtttacatat tgaacctgaa |
| 361 | gaaggtagcc ttgcaggggg aacgtggatc acagtcattt ttgatggttt ggagttgggt |
| 421 | gttctttacc ccaacaatgg ctctcaattg gagatacacc tggtgaacgt gaacatggtg |
| 481 | gtgcccgcac tgcggagtgt tccctgtgac gtctttcctg ttttcttgga tttgcctgtg |
| 541 | gtgacatgcc ggaccagatc tgtgctgtct gaagcacatg agggtctgta cttcctggaa |
| 601 | gcatacttcg ggggacagct ggtaagcagt ccaaatccag gaccacgaga tagctgtact |
| 661 | ttcaagtttt ccaaggcgca gacacccatc gttcaccaag tttatccacc aagtggtgtt |
| 721 | ccaggaaaac taatacatgt atatggctgg attatcactg gaagattgga aacttttgat |
| 781 | tttgatgctg agtacattga tagcccagtg atcttggaag ctcaaggaga caaatgggtt |
| 841 | actccttgct ctcttataaa taggcagatg ggaagctgtt atcctattca ggaggaccat |
| 901 | ggtcttggga ctctgcagtg ccatgtggaa ggcgactaca tcggctccca gaatgttagc |
| 961 | ttctcagtat ttaacaaagg aaagtcaatg gtccacaaga aggcatggct gatcagtgct |
| 1021 | aaacaggatc ttttcctata ccagacacac tcagaaatat tatctgtgtt tccagaaact |
| 1081 | gggagccttg ggggaagaac aaacatcaca attacaggag acttttttga caattctgcc |
| 1141 | caggttacca ttgcaggcat tccatgtgat attagacacg tgtctcccag gaagattgag |
| 1201 | tgcaccactc gggctccagg aaaagatgtg aggctcacca cccctcagcc aggcaatcga |
| 1261 | gggcttcttt ttgaagttgg agatgctgtt gagggactgg aactgactga agccacccca |
| 1321 | gggtacaggt ggcagattgt ccctaatgcc agttctccat ttgggttttg gtcacaggaa |
| 1381 | ggacaacctt tcagagcacg gctcagtggg ttctttgtgg ctccagagac aaataattac |
| 1441 | actttctgga ttcaggcaga tagccaagct tccttgcatt tcagttggtc agaggaacca |
| 1501 | aggactaagg tgaaagtggc ctccatcagc gtcggcactg ctgactggtt tgactcctgg |
| 1561 | gagcagaata gggatgaagg gacctggcag cagaagactc ccaagttgga gctgttgggt |
| 1621 | ggagccatgt actacctgga agcagagcat catgggatag ccccaagcag ggggatgagg |
| 1681 | attggtgtcc agattcacaa cacctggctg aatcctgatg tggtcaccac ttacctacgg |
| 1741 | gagaagcacc agatccgagt ccgagcccag aggcttccag aagtacaggt gctgaatgta |
| 1801 | tcaggcagag gaaacttctt ccttacttgg gacaatgtct ctagtcagcc aatccctgca |
| 1861 | aatgccacag cccatctgat tcaaacaacc attgaggagt tacttgcagt aaaatgcaaa |
| 1921 | ctggaacccc tttggtctaa catccttctc cggcttggat ttgaacgagg cccagaagtt |
| 1981 | tccaactctg atggggacct caccagtggg acggagccct tctgtggcag gttcagcctc |
| 2041 | cgtcagcctc gacaccttgt ccttactccc ccggctgccc agaagggcta tcggctagat |
| 2101 | cagtatacac acctgtgtct tgcatacaaa ggccacatga acaagatcct gaagatgatt |
| 2161 | gtgtccttca caatcggctt tcaaaacatg gtaaagaata ccacctgtga ctggagtctc |
| 2221 | acgaggacca gccccgagag ctggcagttc gattgcactg acctctggga gacttgtgtg |
| 2281 | cgttgcttcg gggatctcca gccccctccg gcaaactccc cagtgctggt tcatcagatc |
| 2341 | aaccttctcc ctctggccca ggagacgggc ctgttctatg tggatgaaat tattattgca |
| 2401 | gacacaaacg taacagtttc tcaagctgat tctggaacgg ctcgcccagg gggcaatctg |
| 2461 | gtggaatcag tctctgtggt gggatcccct ccggtctaca gtgtcacctc ctggctggcg |
| 2521 | gggtgtggca cggagctccc gctcatcact gcacgctctg tgcccactga aggaacagaa |
| 2581 | gagggatctg gactggtcct ggtgacgaca cagagacgac agcggacaag tccacctcta |
| 2641 | ggaggacact ttcgcatcca gcttcctaat acagtgattt ctgatgtccc tgtacaaatt |
| 2701 | tctgctcatc accttcacca gctcttacag aataatgccg atgacttcac atccaggtac |
| 2761 | ctcaatgcca gtgacttcac tgtgaaggag gatctataca cttgctacga acacgtgtgg |
| 2821 | accttgtcct ggtccactca gattggggat ttgcccaatt ttatcagggt ctctgatgaa |
| 2881 | aaccttactg gagtgaatcc tgctgcagcc acgcgtgtgg tatatgatgg tggagttttt |
| 2941 | cttggaccca tatttggaga catgttggct actgccaacc agcatactca ggtggttgtg |
| 3001 | cgagtgaatg atgtaccagc tcattgccca ggttcctgct ctttccagta cctccaaggg |
| 3061 | tcaactccct gtgtccattc tgtgtggtac tccattgatg gtgacatcaa cctaatgatt |
| 3121 | tacattaccg gaactggttt ctctggtgac tcccagttct tgcaggttac agtgaacaaa |
| 3181 | acgagttgca aagttatttt ctcaaaccag accaatgtag tctgtcagac agatttgcta |
| 3241 | cctgttggaa tgcatcggat cttgatgttg gtgagaccct ctggtcttgc catcagtgcc |
| 3301 | actggagaag acctcttcct aaatgtgaaa cctagactgg atatggtgga gccttccaga |
| 3361 | gctgcggata ttggagggct ctgggccacc atccgaggct ctagtttgga aggtgttagc |
| 3421 | ctgatattat ttggatctta ctcgtgtgcc atcaatgtcg ctacaagcaa ttcaagcaga |
| 3481 | attcagtgca aagttccacc cagggggaaa gatggacgca ttgtgaatgt gactgtgatc |
| 3541 | agaggggact attctgcagt tcttcccaga gcatttacat atgtctcttc cttaaatcca |
| 3601 | gttattgtga ctctgagcag aaacataagc aatatagcag gcggtgagac cctggtcatt |
| 3661 | ggagtggcga ggctgatgaa ctatacggat ttggatgtgg aagtccacgt ccaggatgcc |
| 3721 | ttggctccgg ttcacacaca gtcggcttgg ggcctggagg tggcactgcc cccactgcca |
| 3781 | gctggtctcc acagaatttc cgtctctatc aatggggtca gcattcactc acaaggggtt |
| 3841 | gatctccaca tccagtacct cacagaagtt ttcagcatcg agccttgctg tgggtccctg |
| 3901 | ctgggaggga ccatcctcag catctcagga ataggcttca gcagggaccc agctttggtt |
| 3961 | tgggtacttg tgggcaatcg gtcctgtgac attgtgaact taacggaggc gagcatctgg |
| 4021 | tgtgaaaccc tgccagcccc ccagataccc gatgcgggcg ctcccactgt tccagctgcc |
| 4081 | gtggaggtct gggctggcaa caggttcttc gcccgtggtc cttcaccaag cttggtgggg |
| 4141 | aaaggcttca ccttcatgta tgaagcggca gcaacaccag tagtcactgc catgcaagga |
| 4201 | gaaatcacaa atagcagcct gagcctgcat gtgggaggaa gtaacctctc caactcagtc |
| 4261 | atccttctgg ggaacctgaa ctgtgatgtt gagacacagt ccttccaggg caacgtgagc |
| 4321 | ctgtctggat gctccatccc tcttcacagt ctggaggctg gcatctatcc tctccaagta |
| 4381 | cgtcagaagc agatgggatt tgctaatatg tctgtggtgc tccagcaatt tgcagtgatg |
| 4441 | cctcggataa tggccatctt cccatcgcag ggttcggcat gtggtgggac catacttact |
| 4501 | gtgagggggt tgcttcttaa ctctagaagg aggtcagttc gggttgacct ctcgggtcct |
| 4561 | tttacttgtg tgattttgag tttgggagac cacaccattc tctgccaggt tagcctggag |
| 4621 | ggtgacccct tgcctggagc ttccttctcc ctgaacgtca cagtcctggt caatgggcta |
| 4681 | accagcgagt gtcaggggaa ttgcactctt ttcataaggg aagaggcaag tcctgtcatg |
| 4741 | gatgccttgt ccacaaacac cagtgggtct ctgaccactg tgctgattag gggtcagagg |
| 4801 | ttagccacca cagctgatga gccgatggta tttgtggatg atcaacttcc ttgcaatgta |
| 4861 | acttttttta atgcaagcca cgttgtgtgc cagacaagag acttggcccc aggaccccac |
| 4921 | tacctgtcag ttttttatac aagaaatggg tatgcttgtt ctggtaatgt ttccagacac |
| 4981 | ttctacatta tgccccaagt gtttcattat tttcctaaga atttcagctt acatggtgga |
| 5041 | agcctcttga ccatagaggg cacaggcctg agaggacaga acaccacgtc agtctatatt |
| 5101 | gaccagcaga cctgcctgac ggtgaacatc ggtgctgagc tcatccggtg cattgttccc |
| 5161 | acagggaatg gctctgttgc cctggaaata gaggtagatg gactttggta tcacatagga |
| 5221 | gtcattggtt ataacaaggc ctttacccca gaattgatct ctatttctca gagcgatgac |
| 5281 | atcttaacct ttgcagtggc ccagatctca ggagctgcaa acattgacat ttttatagga |
| 5341 | atgtcaccct gtgtgggtgt ctctggtaac cacaccgttc ttcagtgcgt ggtcccttcc |
| 5401 | cttccggccg gggagtacca cgtcagaggc tatgactgca tcagagggtg ggcctcatct |
| 5461 | gccctggtgt tcacctcaag agttattatt acagcagtga cggagaactt cggctgcctg |
| 5521 | ggtggaaggc tggtgcatgt gtttggagcg ggattttctc cagggaatgt ctcagctgct |
| 5581 | gtgtgtggtg ctccctgccg agtcctggct aatgctacag tgtctgcctt cagctgcttg |
| 5641 | gttctgcccc tggatgtgtc cttggccttc ctgtgtggcc tgaagcgtga ggaggacagc |
| 5701 | tgtgaggctg ccagacacac ctatgtgcag tgtgatttga cagttgccat ggcgacagag |
| 5761 | caactgcttg aatcgtggcc ttacctctac atttgcgagg aaagttccca atgcctcttt |
| 5821 | gtgccagatc attgggcaga gtcaatgttt ccatcattct cgggcctctt tatcagccct |
| 5881 | aaattggaaa gagatgaagt tctcatctat aatagctcct gtaacattac catggaaact |
| 5941 | gaggcagaga tggagtgtga gacgcccaat cagccaatta ccgtcaagat tactgagata |
| 6001 | cggaaacgct ggggccagaa cactcagggc aacttttctt tacagttctg ccggagatgg |
| 6061 | tccaggactc acagctggtt tcctgaaagg ctgccacaag atggcgacaa cgtcacagtg |
| 6121 | gagaatggcc aattgcttct gctggacact aacacaagca tcctcaactt actgcacatt |
| 6181 | aaagggggca agctgatttt catggcccca ggacccatcg agctcagggc acacgccatc |
| 6241 | cttgtttctg atggtggaga gctccggatt ggatccgaag acaagccctt ccaaggcaga |
| 6301 | gctcagatca cactctacgg gagttcctac tcaactccct tctttcccta tggagtcaag |
| 6361 | ttcctggctg tgaggaatgg aactctttct ctgcacggtt cactaccaga agtaattgtc |
| 6421 | acctgtctta gagcaactgc ccatgcccta gacacagtgc tggctttaga agatgctgtg |
| 6481 | gactggaacc ctggggatga agttgtcatc atcagtggaa caggtgttaa aggtgccaaa |
| 6541 | ccgatggaag agattgtcac tgtggaaact gtgcaggata cagacctcta tcttaagtca |
| 6601 | cctttgagat attctcacaa ctttacagag aattgggtgg ctggagagca ccatatttta |
| 6661 | aaggccactg tggctctgct cagcaggagt attaccatac aaggaaatct cactaatgag |
| 6721 | agggagaagc tgcttgtttc atgccaggag gccaatgctc cagaaggtaa tctgcagcac |
| 6781 | tgtttgtatt ccatgagtga gaagatgcta ggatccaggg atatgggagc cagagtgatc |
| 6841 | gttcagtcct tcccagaaga gcccagccag gtccagttga agggagtgca gtttcaagtc |
| 6901 | ttggggcaag ccttccataa gcatctgagc tcactcactc tggtgggagc tatgagagag |
| 6961 | tctttcatac agggctgcac agtgaggaac tccttcagta gaggcctcag catgtgcggg |
| 7021 | accttgggcc tgaaggtgga cagtaatgta ttctacaata ttttaggtca tgcgctgcta |
| 7081 | gttgggacat gcacggagat gagatatatc tcctgggagg caattcatgg aaggaaagat |
| 7141 | gactggtcag gacatggaaa tataataaga aacaacgtga tcatccaggt ttctggtgcc |
| 7201 | gagggactct ccaatcctga aatgttgaca ccatctggca tctatatctg cagtcccacc |
| 7261 | aatgttatag aggggaacag agtgtgtggt gctggctatg gctacttttt ccatctcatg |
| 7321 | accaaccaaa catcacaagc tccgcttctt tccttcactc agaacattgc acattcttgt |
| 7381 | accaggtatg gtctctttgt ataccctaaa tttcagccac cttgggataa tgtcactggc |
| 7441 | accactctgt tccagagctt cacagtttgg gaaagtgcag gtggtgccca gatttttaga |
| 7501 | agtagcaatc ttcgcctgaa aaacttcaaa gtttattcat gcagagattt tggaattgac |
| 7561 | gtcttggaaa gtgatgcaaa tacttcagtt actgacagct tattacttgg tcattttgcc |
| 7621 | cacaagggaa gtctgtgtat gtcatctggg attaaaactc ctaaaagatg ggaactgatg |
| 7681 | gtgtctaaca caacctttgt taattttgat ctcatcaact gtgtggccat tagaacctgt |
| 7741 | tcagactgtt cccaaggaca aggtggattt actgtgaaga ccagccagtt gaagtttaca |
| 7801 | aactcttcaa acttagtggc atttccattt cctcatgcag caattttgga agacttggat |
| 7861 | gggtctctgt ctgggaaaaa cagaagtcac attcttgctt ctatggaaac cctttcagct |
| 7921 | tcttgtttgg tcaattcaag ctttggtcgg gttgtccatg gcagtgcctg tggaggaggt |
| 7981 | gttctttttc atcgtatgtc tattggttta gcgaatactc ctgaagtttc ttatgattta |
| 8041 | accatgactg acagcagaaa taaaacaacc actgtcaatt atgtacgtga tacattgtct |
| 8101 | aaccctcgtg gctggatggc tctgctcttg gaccaagaga cctactcatt gcaatctgag |
| 8161 | aacctttgga tcaacagatc tctgcagtac tcagcaacct ttgacaactt tgctcctggt |
| 8221 | aattacctac tgctggtgca cacagatttg ccgccttacc ctgacatcct cctaagatgt |
| 8281 | gggagtcgag tgggtctgtc ttttccattt cttccatcac caggtcagaa ccaaggctgt |
| 8341 | gactggttct tcaatagcca gctgaggcaa ctcacctatc tggtttcagg tgaaggccaa |
| 8401 | gttcaagtca ttctccgggt gaaggaaggt atgcccccaa ctatttcagc ttctacctct |
| 8461 | gcccctgaat cagctttaaa atggtccctc cctgaaacat ggcaaggtgt tgaagaaggc |
| 8521 | tggggaggat acaacaatac cattccaggc cctggggatg acgttctcat tttacccaac |
| 8581 | agaactgtcc ttgtggatac agatcttcca ttcttcaaag ggctgtatgt gatggggacc |
| 8641 | ttagacttcc ctgtggacag aagcaatgtt ctgagtgtgg catgcatggt cattgcaggc |
| 8701 | ggggagctga aagttggtac tttagaaaat cccttagaaa aggaacaaaa gcttctgatt |
| 8761 | ctccttagag cctcagaggg agtcttttgt gaccgtatga atggaattca tattgaccca |
| 8821 | ggaacaattg gggtttatgg gaaagttcat ctttacagtg cttatcctaa gaactcctgg |
| 8881 | acacatcttg gagctgatat tgcctcagga aatgagagaa ttatagtaga agatgcagtg |
| 8941 | gattggcgcc cccatgacaa aatagtcctt agctcctctt cttatgagcc tcatgaagca |
| 9001 | gaggtcctca ctgtgaaaga agtcaagggc caccatgtga ggatctatga acggctcaaa |
| 9061 | caccggcata ttggaagtgt acatgtcacg gaggatggcc gacacattcg tttggctgct |
| 9121 | gaggttggac tgttgacccg aaatatacaa attcagcctg acgtatcatg tagggggaga |
| 9181 | ctgtttgtgg ggtccttcag gaagtccagc cgagaagaat tttcaggtgt ccttcaactt |
| 9241 | cttaatgtgg aaattcagaa cttcgggtca ccattgtact catctgttga attcagtaat |
| 9301 | gtgtcagcag gatcctggat catatcatct actctgcacc agagctgtgg cgggggcatt |
| 9361 | catgcagctg ccagtcatgg agtactttta aatgacaata ttgtgtttgg cacagctggc |
| 9421 | catggcatag atttagaggg tcaggcctat actgtcacta ataaccttgt ggttctgatg |
| 9481 | acacagccag cgtggtccac catttgggtg gcgggaatca aagtgaacca ggtaaaggac |
| 9541 | atcaacctcc atggcaacgt tgtggcagga tcagagagac ttggctttca catccgaggc |
| 9601 | cacaagtgct cctcttgtga actgctttgg tctgacaatg tggcgcattc aagtcttcat |
| 9661 | ggccttcatc tctataagga aagtggactt gacaactgta ccagaatctc tggcttcttg |
| 9721 | gctttcaaga actttgacta tggtgccatg ttacatgtag agaacagcgt ggagatagag |
| 9781 | aacattactc tggtagacaa tactattggt cttttggcag tagtgtatgt attttctgct |
| 9841 | ccacaaaatt ccgtcaaaaa agtgcagatt gtgcttagga attcagtcat tgtggccacc |
| 9901 | agctcttctt ttgactgcat tcaggacaaa gtgaagccgc actcagccaa cttgacatca |
| 9961 | acagatagag ctccctccaa tccaagagga ggtcgaattg gtattctgtg gcctgtattc |
| 10021 | acctcagaac caaatcagtg gcctcaggag ccatggcaca aagtgaggaa tgatcattca |
| 10081 | atttcaggaa tcatgaaact tcaagatgtt accttttcta gttttgtgaa gagttgctat |
| 10141 | agcgatgacc tggatgtctg cattctacca aatgcagaga acagtggaat tatgcaccca |
| 10201 | ataacagcag agaggaccag gatgctaaag ataaaagata aaaacaagtt ctactttcct |
| 10261 | tcattacaac ccaggaaaga tttaggaaaa gtagtctgtc ctgaattaga ctgtgcaagt |
| 10321 | ccaagaaaat atctcttcaa ggatctggat gggagagccc tgggtctgcc tccaccagtt |
| 10381 | tctgtatttc ctaaaacaga ggcagaatgg actgcatcct tcttcaacgc aggtacattt |
| 10441 | agagaagaac agaaatgtac ataccaattt ctgatgcaag gattcatctg caaacagact |
| 10501 | gaccaagtgg tcctaattct tgatagcgct gatgccattt gggcaattca gaagttatat |
| 10561 | ccagttgtat ctgtgactag tggttttgtt gatgtcttta gcagtgtaaa tgccaatatt |
| 10621 | ccctgctcta cttctgggtc agtgtctact ttctattcta tcttacccat caggcaaatc |
| 10681 | accaaagtct gcttcatgga tcaaactcct caagttttgc gcttttttct attggggaac |
| 10741 | aaaagtacct ccaagcttct cttggctgta ttctaccatg agctccagag cccccacgtc |
| 10801 | ttcttagggg aaagttttat tccacccact ctggttcagt cagcttcctt attgctgaat |
| 10861 | gaatctattg gtgccaacta tttcaacatc atggataacc tcttgtatgt tgtcctacaa |
| 10921 | ggagaggagc ccattgaaat acgctcaggt gtttccattc acttggccct cactgtgatg |
| 10981 | gtttcagtct tagaaaaagg ctgggaaata gtaatactcg aaagactaac taacttctta |
| 11041 | cagattggcc aaaaccaaat caggtttatt cacgagatgc ctggccatga agagacctta |
| 11101 | aaggccattg ctgacagtag agcaaaaaga aagcgcaatt gccctactgt gacttgcact |
| 11161 | agtcattata gaagagttgg tcaacgtagg cctctcatga tggaaatgaa ctcacatagg |
| 11221 | gcttcacccc caatgactgt ggaaactatc tcaaaagtga ttgtcattga aattggtgat |
| 11281 | tcgccaacag taaggagcac tggaatgatt tcatccttat caagtaacaa attacagaat |
| 11341 | ttggctcatc gagtcatcac tgctcaacag actggggtac tagagaatgt tctgaatatg |
| 11401 | actatcgggg ccttactagt tactcagtca aagggagtca ttggctatgg aaatacaagc |
| 11461 | agttttaaaa ctgggaactt gatatatatt cggccctatg cactttccat cctagtccag |
| 11521 | ccttcagatg gagaagtggg aaatgagctt ccagtgcagc cacaattggt atttttggat |
| 11581 | gagcagaatc gaagagtaga gtccctggga cctccttcag agccatggac aatttcagct |
| 11641 | tccctggaag gagcatcaga ctcagtgcta aaagggtgca cccaggcaga aactcaagat |
| 11701 | ggttatgtta gcttctacaa cttggcagtc ttgatctctg ggtcaaactg gcactttatt |
| 11761 | tttactgtca cttctcctcc aggagtcaat tttacagctc gatccaagcc atttgctgtc |
| 11821 | ttgcctgtga ctaggaagga gaagtcgacc atcatcctgg ctgcttccct gtcctctgtg |
| 11881 | gcctcatggc tggctctgag ctgtctggtg tgctgttggc ttaaaagaag caaaagcaga |
| 11941 | aaaacaaaac ctgaagagat tcctgaatcc cagactaata atcaaaatat tcatatccac |
| 12001 | atctcatcca aacgccgaga atcacaaggg cccaaaaaag aagacactgt ggtgggagaa |
| 12061 | gatatgagaa tgaaggtcat gctgggcaag gtgaaccagt gcccccacca gttgatgaat |
| 12121 | ggagtgtcca gaaggaaagt tagccgccac attgtccgag aggaagaggc tgctgtgcct |
| 12181 | gctcctggta ctactggcat cacatcccat gggcacatct gtgctccagg tgctcctgct |
| 12241 | cagcaggtgt acctgcaaga gactgggaac tggaaggagg gccaagagca gttgctcaga |
| 12301 | taccagctgg caggccaaaa tcagctgctg ctgctatgcc cagacttcag acaagagagg |
| 12361 | cagcagttgc cagggcaaag tcggctgagt aagcaaagtg gcagcttggg gctttcccaa |
| 12421 | gagaagaaag cctcctgcgg ggccactgag gcattctgcc ttcattcagt acacccggaa |
| 12481 | actattcagg agcaactgtg atcagggaag ttgggggcat ttggcctgaa aggcagaatg |
| 12541 | ttcccagtat ttctggataa taagctgggg aagtgaggac tgtcctgctg ggacaactaa |
| 12601 | gaagagagaa tgtggactct gaatcccttt ttcaacttta aaatggaaaa cagtcatata |
| 12661 | aatgcttaca gactgaaaaa tgtctcatac atattttgca gctatcatga tcctagttca |
| 12721 | atgctaggca aataatggca cttgttaatt atttaccagt ttttaactta agcctgattt |
| 12781 | taacaacttg ataatggtgt aatactgact tataccagca ggggttatta ttaagcattc |
| 12841 | tctggattga ccacccacaa tgctttgagc ttcttttata gaagggatca tgaaaaggtc |
| 12901 | tggtcagcta cagtttaatt ccacactgtt acagaaaagc atttctttat cctgtagtag |
| 12961 | catttgaagg gacagttcaa caccctgctc cctgatctgt cagagcttac cttccaacca |
| 13021 | caaggcaact tcctggcttt ttacaagtgg attttatttc atggtttaaa ttcaatcatt |
| 13081 | attgagaacc tacagtagtg tgagcgtatc aaatttgcac ttcatgagga tttacacatg |
| 13141 | aaaattgata ttctcatggg ctttgctgag agtttagtta aggacaaaga tgtaaaaatg |
| 13201 | ccaaatttgg gaaaggaaat tttgtatgac cactgttcta tttttcagtg attctctctg |
| 13261 | ttagtagctg ggtcacattt acaactggaa aagaactaca ttgggagatc agagaaagcc |
| 13321 | aagtggccaa gtcctttatc aaaaagctgc tttttggaga agatgatgca taaagctgtg |
| 13381 | atactaagga gaggagagat tatgtcatat gcaactgctc taggctgtgc agggggttta |
| 13441 | aatggagatc agaacaaagg ctcacctggt attgatacat ttggtaaggt tgttaaaggc |
| 13501 | aatttcacca aattcacaca tttgtacggg gcccatttct tttcaaactg aagaatactt |
| 13561 | taaacagcct gactgtgtcc ttggctctgg atccagaagg ccttatgctc caagcatgta |
| 13621 | aagaaaagat cgcaggaatg tggagagctt ccaaaaagag attgctctca gattttcctg |
| 13681 | aaaatcctga aaaccaggat tttccaatct ctacaatgtt aaggggttgt tgtgtagttg |
| 13741 | cattggaaga gagaaaggaa acacactctt tcattgctga attctaaata atgatgccct |
| 13801 | gaattagttc tatgagaagt actggcagac agctgttatg tctgcagggc atcaaggggc |
| 13861 | aggtgtgtct caccctacta agaattggca gaaatgtttt aacctcttct ttaggcatct |
| 13921 | ggtgagtatt gcagatcttc aggctgtccc taaaaggagg taggtagcct tctctgtggt |
| 13981 | cactgtgttg gatttggaag aaaatataag tagatgttaa ggtcagtgct ctccaggaga |
| 14041 | tttcaatcca ggctatctgt gttcagggaa tgatttgcaa gaagttggtt tatagacgtt |
| 14101 | tactctggag gcacaagctg gctgccatgc tgagtaagtc ctctttgtag atgatgtatt |
| 14161 | ttgcttgggc accaacagaa ttaaaaacga aaaacaaaaa aaaactcgaa tgacttaggc |
| 14221 | agaacatgcc ttccctagtt taattagtcc ctgttataac cagttattta tctttttttg |
| 14281 | tagattctcg gtcttgaagt catttgcctt tgcaatctct actttagtat actcattgga |
| 14341 | tcctcaaaga gccgtaagct ttttggaaaa aaacatttca gaagtgaagg cttttgaggt |
| 14401 | ttataaaatg gtagcagtct tagtccttgc cctgcctcag tttacctgca catgctttct |
| 14461 | ttcatttcct cggattcttt caccactttt taagtttttt ttccacaggt atttttcctg |
| 14521 | agctctatac catgcattta gctgcctagt gagaatatcc agtaggttat cctatagtta |
| 14581 | ctatgaagtt aacacagccc aaaaacctac ttctccttgt gtttttttcc aactcaatga |
| 14641 | atgacatcac tgcactcaga ccagaaatct agaagtcagt catgactctt cattctattt |
| 14701 | cgcccttcac aaagagttag ctgccagctc tgttatttct gccccttctt tacccttctt |
| 14761 | ccaattttta ttatcttctt atttatcctc tatgtgtcac aagaccagtc tttctgggag |
| 14821 | gcatgtttta tcctatccct ctgcgtagga ataaccataa ttatcttact aattccatcc |
| 14881 | cagaaaagcc tgtgatccct gaggccagaa ttgactcttt gggcctaaat agcagctata |
| 14941 | aacatcttca gtaacttgcc aggctctagc atgtggcatg gaattttggc atctagctaa |
| 15001 | aattcctcaa cccatagtta agctactcct ctttctccta aaaaaaaaaa aaaatgagag |
| 15061 | aattagggaa acaccaaaag caaagagagc agctctatgt tcattcatgt ctggagatga |
| 15121 | gccaatttga gcaccactgt aggccaactc taaatttatt cagatcgtta acatgggtaa |
| 15181 | cccagcaaat atttcccagc agtgacaatc tacacccttt atctaaataa atattgagat |
| 15241 | gtgctttttt cctgtgttag ttcaaagaat ctcttcttgc ctcccattgc tgattttctg |
| 15301 | tattgatgga ctcatttctc tcaagagatt ggacaagcta aaattctaga gagttcaaaa |
| 15361 | gtgtttgaaa gtacatttta agtgttctca ccacaaaaaa tgctagtgtg tgaggtgata |
| 15421 | catatgttaa ttagttcaac tgatccattc cacaatgtat acatatttca aactatcgag |
| 15481 | ttgtacatga taaatatata caatttttat ttgtcaaata aagaacatct ataaatattt |
| 15541 | atagatttca aaattgagaa agaagtatat atggagccac ggttgttttg accatttctc |
| 15601 | catgcttttt gtttggggag ggggacattt tttggggggc agggactgtg ttcagggaaa |
| 15661 | gtacctgacc aacagaactt caagtctctt ccagagtgcg agactcagct tatctgatca |
| 15721 | aggtgaactt gcccttcaat agacttgccc ttgttactgc ccagtgaggc caccaaggac |
| 15781 | caagaacagt tgagctctct cagggccttc ccctttcaac aatttagttg aatgactccc |
| 15841 | ttccatctat cagtcttact ccaaatatat gaaattacta tacattgaaa tgcctgcaaa |
| 15901 | ctatatgcaa aaatgagtat gaggaaatca aatgtaaatc atatgcaaat attattcaac |
| 15961 | caagcttcta taaaattgta cgtgtcaata acagtaaaga attttaatta attctgtcat |
| 16021 | ttaaaattca tgcattccat attttgcttt cagaacttat agtctatgcc aattttgaaa |
| 16081 | aaactataag cacaaggaat taaatactat tgagtataga actgttgatt tcaacataca |
| 16141 | ctatagcact ttgactaatt acagatatat ttgatagaaa agtcacattt ctacaaggta |
| 16201 | attttcaaaa agaaataaaa catttctgta aactt |

PKD1 (SEQ ID NO: 85) - Homo sapiens polycystin kidney disease 1,
transient receptor potential channel interacting (PKD1), transcript
variant 2, mRNA - NM_000296

| | |
|---|---|
| 1 | gcactgcagc gccagcgtcc gagcgggcgg ccgagctccc ggagcggcct ggccccgagc |
| 61 | cccgagcggg cgtcgctcag cagcaggtcg cggccgcagc cccatccagc cccgcgcccg |
| 121 | ccatgccgtc cgcgggcccc gcctgagctg cggcctccgc gcgcgggcgg gcctggggac |
| 181 | ggcggggcca tgcgcgcgct gccctaacga tgccgcccgc cgcgcccgcc cgcctggcgc |
| 241 | tggccctggg cctgggcctg tggctcgggg cgctggcggg gggccccggg cgcggctgcg |
| 301 | ggccctgcga gcccccctgc ctctgcggcc cagcgcccgg cgccgcctgc cgcgtcaact |
| 361 | gctcgggccg cgggctgcgg acgctcggtc ccgcgctgcg catccccgcg gacgccacag |
| 421 | cgctagacgt ctcccacaac ctgctccggg cgctggacgt tgggctcctg gcgaacctct |
| 481 | cggcgctggc agagctggat ataagcaaca acaagatttc tacgttagaa gaaggaatat |
| 541 | ttgctaattt atttaattta agtgaaataa acctgagtgg gaacccgttt gagtgtgact |
| 601 | gtggcctggc gtggctgccg cgatgggcgg aggagcagca ggtgcgggtg gtgcagcccg |
| 661 | aggcagccac gtgtgctggg cctggctccc tggctggcca gcctctgctt ggcatcccct |
| 721 | tgctggacag tggctgtggt gaggagtatg tcgcctgcct ccctgacaac agctcaggca |
| 781 | ccgtggcagc agtgtccttt tcagctgccc acgaaggcct gcttcagcca gaggcctgca |
| 841 | gcgccttctg cttctccacc ggccagggcc tcgcagccct ctcggagcag ggctggtgcc |
| 901 | tgtgtggggc ggcccagccc tccagtgcct cctttgcctg cctgtccctc tgctccggcc |
| 961 | ccccgccacc tcctgccccc acctgtaggg gccccaccct cctccagcac gtcttccctg |
| 1021 | cctccccagg ggccaccctg gtggggcccc acggacctct ggcctctggc cagctagcag |
| 1081 | ccttccacat cgctgccccg ctccctgtca ctgccacacg ctgggacttc ggagacggct |
| 1141 | ccgccgaggt ggatgccgct gggccggctg cctcgcatcg ctatgtgctg cctgggcgct |
| 1201 | atcacgtgac ggccgtgctg gccctggggg ccggctcagc cctgctgggg acagacgtgc |
| 1261 | aggtggaagc ggcacctgcc gccctggagc tcgtgtgccc gtcctcggtg cagagtgacg |
| 1321 | agagccttga cctcagcatc cagaaccgcg gtggttcagg cctggaggcc gcctacagca |
| 1381 | tcgtggccct gggcgaggag ccggcccgag cggtgcaccc gctctgcccc tcggacacgg |
| 1441 | agatcttccc tggcaacggg cactgctacc gcctggtggt ggagaaggcg gcctggctgc |
| 1501 | aggcgcagga gcagtgtcag gcctgggccg gggccgccct ggcaatggtg gacagtcccg |
| 1561 | ccgtgcagcg cttcctggtc tcccgggtca ccaggagcct agacgtgtgg atcggcttct |
| 1621 | cgactgtgca gggggtggag gtgggcccag cgccgcaggg cgaggccttc agcctggaga |
| 1681 | gctgccagaa ctggctgccc ggggagccac acccagccac agccgagcac tgcgtccggc |
| 1741 | tcgggcccac cgggtggtgt aacaccgacc tgtgctcagc gccgcacagc tacgtctgcg |
| 1801 | agctgcagcc cggaggccca gtgcaggatg ccgagaacct cctcgtggga gcgcccagtg |
| 1861 | gggacctgca gggacccctg acgcctctgg cacagcagga cggcctctca gccccgcacg |
| 1921 | agcccgtgga ggtcatggta ttcccgggcc tgcgtctgag ccgtgaagcc ttcctcacca |
| 1981 | cggccgaatt tgggacccag gagctccggc ggcccgccca gctgcggctg caggtgtacc |
| 2041 | ggctcctcag cacagcaggg accccggaga acggcagcga gcctgagagc aggtccccgg |
| 2101 | acaacaggac ccagctggcc cccgcgtgca tgccaggggg acgctggtgc cctggagcca |
| 2161 | acatctgctt gccgctggac gcctcctgcc acccccaggc ctgcgccaat ggctgcacgt |
| 2221 | cagggccagg gctacccggg gccccctatg cgctatggag agagttcctc ttctccgttc |
| 2281 | ccgcggggcc ccccgcgcag tactcggtca ccctccacgg ccaggatgtc ctcatgctcc |
| 2341 | ctggtgacct cgttggcttg cagcacgacg ctggccctgg cgccctcctg cactgctcgc |
| 2401 | cggctcccgg ccaccctggt ccccaggccc cgtacctctc cgccaacgcc tcgtcatggc |
| 2461 | tgccccactt gccagcccag ctggagggca cttgggcctg ccctgcctgt gccctgcggc |
| 2521 | tgcttgcagc cacggaacag ctcaccgtgc tgctgggctt gaggcccaac cctggactgc |
| 2581 | ggctgcctgg gcgctatgag gtccgggcag aggtgggcaa tggcgtgtcc aggcacaacc |
| 2641 | tctcctgcag ctttgacgtg gtctccccag tggctgggct gcgggtcatc taccctgccc |
| 2701 | cccgcgacgg ccgcctctac gtgcccacca acggctcagc cttggtgctc caggtggact |
| 2761 | ctggtgccaa cgccacggcc acggctcgct ggcctggggg cagtgtcagc gcccgctttg |
| 2821 | agaatgtctg ccctgccctg gtggccacct tcgtgcccgg ctgcccctgg gagaccaacg |
| 2881 | ataccctgtt ctcagtggta gcactgccgt ggctcagtga gggggagcac gtggtggacg |
| 2941 | tggtggtgga aaacagcgcc agccgggcca acctcagcct gcgggtgacg gcggaggagc |
| 3001 | ccatctgtgg cctccgcgcc acgcccagcc ccgaggcccg tgtactgcag ggagtcctag |
| 3061 | tgaggtacag ccccgtggtg gaggccggct cggacatggt cttccggtgg accatcaacg |
| 3121 | acaagcagtc cctgaccttc cagaacgtgg tcttcaatgt catttatcag agcgcggcgg |
| 3181 | tcttcaagct ctcactgacg gcctccaacc acgtgagcaa cgtcaccgtg aactacaacg |
| 3241 | taaccgtgga gcggatgaac aggatgcagg gtctgcaggt ctccacagtg ccggccgtgc |
| 3301 | tgtcccccaa tgccacgcta gcactgacgg cgggcgtgct ggtggactcg gccgtggagg |
| 3361 | tggccttcct gtggaccttt ggggatgggg agcaggccct ccaccagttc cagcctccgt |
| 3421 | acaacgagtc cttcccggtt ccagacccct cggtggccca ggtgctggtg gagcacaatg |
| 3481 | tcatgcacac ctacgctgcc ccaggtgagt acctcctgac cgtgctggca tctaatgcct |
| 3541 | tcgagaacct gacgcagcag gtgcctgtga gcgtgcgcgc ctccctgccc tccgtggctg |
| 3601 | tgggtgtgag tgacggcgtc ctggtggccg gccggcccgt caccttctac ccgcacccgc |
| 3661 | tgccctcgcc tgggggtgtt ctttacacgt gggacttcgg ggacggctcc cctgtcctga |
| 3721 | cccagagcca gccggctgcc aaccacacct atgcctcgag gggcacctac cacgtgcgcc |
| 3781 | tggaggtcaa caacacggtg agcggtgcgg cggcccaggc ggatgtgcgc gtctttgagg |
| 3841 | agctccgcgg actcagcgtg gacatgagcc tggccgtgga gcagggcgcc cccgtggtgg |
| 3901 | tcagcgccgc ggtgcagacg ggcgacaaca tcacgtggac cttcgacatg ggggacggca |
| 3961 | ccgtgctgtc gggcccggag gcaacagtgg agcatgtgta cctgcgggca cagaactgca |
| 4021 | cagtgaccgt gggtgcggcc agccccgccg gccacctggc ccggagcctg cacgtgctgg |
| 4081 | tcttcgtcct ggaggtgctg cgcgttgaac ccgccgcctg catccccacg cagcctgacg |
| 4141 | cgcggctcac ggcctacgtc accgggaacc cggcccacta cctcttcgac tggaccttcg |
| 4201 | gggatggctc ctccaacacg accgtgcggg ggtgcccgac ggtgacacac aacttcacgc |
| 4261 | ggagcggcac gttccccctg gcgctggtgc tgtccagccg cgtgaacagg gcgcattact |
| 4321 | tcaccagcat ctgcgtggag ccagaggtgg gcaacgtcac cctgcagcca gagaggcagt |
| 4381 | ttgtgcagct cggggacgag gcctggctgg tggcatgtgc ctggcccccg ttcccctacc |
| 4441 | gctacacctg ggactttggc accgaggaag ccgcccccac ccgtgccagg ggccctgagg |
| 4501 | tgacgttcat ctaccgagac ccaggctcct atcttgtgac agtcaccgcg tccaacaaca |
| 4561 | tctctgctgc caatgactca gccctggtgg aggtgcagga gcccgtgctg gtcaccagca |
| 4621 | tcaaggtcaa tggctccctt gggctggagc tgcagcagcc gtacctgttc tctgctgtgg |
| 4681 | gccgtgggcg ccccgccagc tacctgtggg atctggggga cggtgggtgg ctcgagggtc |
| 4741 | cggaggtcac ccacgcttac aacagcacag gtgacttcac cgttagggtg gccggctgga |
| 4801 | atgaggtgag ccgcagcgag gcctggctca atgtgacggt gaagcggcgc gtgcgggggc |
| 4861 | tcgtcgtcaa tgcaagccgc acggtggtgc ccctgaatgg gagcgtgagc ttcagcacgt |
| 4921 | cgctggaggc cggcagtgat gtgcgctatt cctgggtgct ctgtgaccgc tgcacgccca |
| 4981 | tccctggggg tcctaccatc tcttacacct tccgctccgt gggcaccttc aatatcatcg |
| 5041 | tcacggctga gaacgaggtg ggctccgccc aggacagcat cttcgtctat gtcctgcagc |
| 5101 | tcatagaggg gctgcaggtg gtgggcggtg gccgctactt ccccaccaac cacacggtac |
| 5161 | agctgcaggc cgtggttagg gatggcacca acgtctccta cagctggact gcctggaggg |
| 5221 | acaggggccc ggccctggcc ggcagcggca aaggcttctc gctcaccgtg ctcgaggccg |
| 5281 | gcacctacca tgtgcagctg cgggccacca acatgctggg cagcgcctgg gccgactgca |
| 5341 | ccatggactt cgtggagcct gtggggtggc tgatggtggc cgcctccccg aacccagctg |
| 5401 | ccgtcaacac aagcgtcacc ctcagtgccg agctggctgg tggcagtggt gtcgtataca |
| 5461 | cttggtcctt ggaggagggg ctgagctggg agacctccga gccatttacc acccatagct |
| 5521 | tccccacacc cggcctgcac ttggtcacca tgacggcagg gaacccgctg ggctcagcca |
| 5581 | acgccaccgt ggaagtggat gtgcaggtgc ctgtgagtgg cctcagcatc agggccagcg |
| 5641 | agcccggagg cagcttcgtg gcggccgggt cctctgtgcc cttttggggg cagctggcca |
| 5701 | cgggcaccaa tgtgagctgg tgctgggctg tgcccggcgg cagcagcaag cgtggccctc |
| 5761 | atgtcaccat ggtcttcccg gatgctggca ccttctccat ccggctcaat gcctccaacg |
| 5821 | cagtcagctg ggtctcagcc acgtacaacc tcacggcgga ggagcccatc gtgggcctgg |
| 5881 | tgctgtgggc cagcagcaag gtggtggcgc ccgggcagct ggtccatttt cagatcctgc |
| 5941 | tggctgccgg ctcagctgtc accttccgcc tgcaggtcgg cggggccaac cccgaggtgc |
| 6001 | tccccgggcc ccgtttctcc cacagcttcc cccgcgtcgg agaccacgtg gtgagcgtgc |
| 6061 | ggggcaaaaa ccacgtgagc tgggcccagg cgcaggtgcg catcgtggtg ctggaggccg |
| 6121 | tgagtgggct gcaggtgccc aactgctgcg agcctggcat cgccacgggc actgagagga |
| 6181 | acttcacagc ccgcgtgcag cgcggctctc gggtcgccta cgcctggtac ttctcgctgc |
| 6241 | agaaggtcca gggcgactcg ctggtcatcc tgtcgggccg cgacgtcacc tacacgcccg |
| 6301 | tggccgcggg gctgttggag atccaggtgc gcgccttcaa cgccctgggc agtgagaacc |
| 6361 | gcacgctggt gctggaggtt caggacgccg tccagtatgt ggccctgcag agcggcccct |
| 6421 | gcttcaccaa ccgctcggcg cagtttgagg ccgccaccag ccccagcccc cggcgtgtgg |
| 6481 | cctaccactg ggactttggg gatgggtcgc cagggcagga cacagatgag cccagggccg |
| 6541 | agcactccta cctgaggcct ggggactacc gcgtgcaggt gaacgcctcc aacctggtga |
| 6601 | gcttcttcgt ggcgcaggcc acggtgaccg tccaggtgct ggcctgccgg gagccggagg |
| 6661 | tggacgtggt cctgcccctg caggtgctga tgcggcgatc acagcgcaac tacttggagg |
| 6721 | cccacgttga cctgcgcgac tgcgtcacct accagactga gtaccgctgg gaggtgtatc |
| 6781 | gcaccgccag ctgccagcgg ccggggcgcc cagcgcgtgt ggccctgccc ggcgtggacg |
| 6841 | tgagccggcc tcggctggtg ctgccgcggc tggcgctgcc tgtggggcac tactgctttg |
| 6901 | tgtttgtcgt gtcatttggg gacacgccac tgacacagag catccaggcc aatgtgacgg |
| 6961 | tggcccccga gcgcctggtg cccatcattg agggtggctc ataccgcgtg tggtcagaca |
| 7021 | cacgggacct ggtgctggat gggagcgagt cctacgaccc caacctggag gacggcgacc |
| 7081 | agacgccgct cagtttccac tgggcctgtg tggcttcgac acagagggag gctggcgggt |
| 7141 | gtgcgctgaa ctttgggccc cgcgggagca gcacggtcac cattccacgg gagcggctgg |
| 7201 | cggctggcgt ggagtacacc ttcagcctga ccgtgtggaa ggccggccgc aaggaggagg |
| 7261 | ccaccaacca gacggtgctg atccggagtg gccgggtgcc cattgtgtcc ttggagtgtg |
| 7321 | tgtcctgcaa ggcacaggcc gtgtacgaag tgagccgcag ctcctacgtg tacttggagg |
| 7381 | gccgctgcct caattgcagc agcggctcca agcgagggcg gtgggctgca cgtacgttca |
| 7441 | gcaacaagac gctggtgctg gatgagacca ccacatccac gggcagtgca ggcatgcgac |
| 7501 | tggtgctgcg gcggggcgtg ctgcgggacg gcgagggata caccttcacg ctcacggtgc |
| 7561 | tgggccgctc tggcgaggag gagggctgcg cctccatccg cctgtccccc aaccgcccgc |
| 7621 | cgctgggggg ctcttgccgc ctcttcccac tgggcgctgt gcacgccctc accaccaagg |
| 7681 | tgcacttcga atgcacgggc tggcatgacg cggaggatgc tggcgccccg ctggtgtacg |
| 7741 | ccctgctgct gcggcgctgt cgccagggcc actgcgagga gttctgtgtc tacaagggca |
| 7801 | gcctctccag ctacggagcc gtgctgcccc cgggtttcag gccacacttc gaggtgggcc |
| 7861 | tggccgtggt ggtgcaggac cagctgggag ccgctgtggt cgccctcaac aggtctttgg |
| 7921 | ccatcaccct cccagagccc aacggcagcg caacggggct cacagtctgg ctgcacgggc |
| 7981 | tcaccgctag tgtgctccca gggctgctgc ggcaggccga tccccagcac gtcatcgagt |
| 8041 | actcgttggc cctggtcacc gtgctgaacg agtacgagcg ggccctggac gtggcggcag |
| 8101 | agcccaagca cgagcggcag caccgagccc agatacgcaa gaacatcacg gagactctgg |
| 8161 | tgtccctgag ggtccacact gtggatgaca tccagcagat cgctgctgcg ctggcccagt |
| 8221 | gcatggggcc cagcagggag ctcgtatgcc gctcgtgcct gaagcagacg ctgcacaagc |
| 8281 | tggaggccat gatgctcatc ctgcaggcag agaccaccgc gggcaccgtg acgcccaccg |
| 8341 | ccatcggaga cagcatcctc aacatcacag gagacctcat ccacctggcc agctcggacg |
| 8401 | tgcgggcacc acagccctca gagctgggag ccgagtcacc atctcggatg gtggcgtccc |
| 8461 | aggcctacaa cctgacctct gccctcatgc gcatcctcat gcgctcccgc gtgctcaacg |
| 8521 | aggagcccct gacgctggcg ggcgaggaga tcgtggccca gggcaagcgc tcggacccgc |
| 8581 | ggagcctgct gtgctatggc ggcgccccag ggcctggctg ccacttctcc atccccgagg |
| 8641 | ctttcagcgg ggccctggcc aacctcagtg acgtggtgca gctcatcttt ctggtggact |
| 8701 | ccaatccctt tccctttggc tatatcagca actacaccgt ctccaccaag gtggcctcga |
| 8761 | tggcattcca gacacaggcc ggcgcccaga tccccatcga gcggctggcc tcagagcgcg |
| 8821 | ccatcaccgt gaaggtgccc aacaactcgg actgggctgc ccggggccac cgcagctccg |
| 8881 | ccaactccgc caactccgtt gtggtccagc cccaggcctc cgtcggtgct gtggtcaccc |
| 8941 | tggacagcag caaccctgcg gccgggctgc atctgcagct caactatacg ctgctggacg |
| 9001 | gccactacct gtctgaggaa cctgagccct acctggcagt ctacctacac tcggagcccc |
| 9061 | ggcccaatga gcacaactgc tcggctagca ggaggatccg cccagagtca ctccagggtg |
| 9121 | ctgaccaccg gccctacacc ttcttcattt ccccggggag cagagaccca gcggggagtt |
| 9181 | accatctgaa cctctccagc cacttccgct ggtcggcgct gcaggtgtcc gtgggcctgt |
| 9241 | acacgtccct gtgccagtac ttcagcgagg aggacatggt gtggcggaca gaggggctgc |
| 9301 | tgcccctgga ggagacctcg ccccgccagg ccgtctgcct cacccgccac ctcaccgcct |
| 9361 | tcggcgccag cctcttcgtg cccccaagcc atgtccgctt tgtgtttcct gagccgacag |
| 9421 | cggatgtaaa ctacatcgtc atgctgacat gtgctgtgtg cctggtgacc tacatggtca |
| 9481 | tggccgccat cctgcacaag ctggaccagt tggatgccag ccggggccgc gccatccctt |
| 9541 | tctgtgggca gcggggccgc ttcaagtacg agatcctcgt caagacaggc tggggccggg |
| 9601 | gctcaggtac cacggcccac gtgggcatca tgctgtatgg ggtggacagc cggagcggcc |
| 9661 | accggcacct ggacggcgac agagccttcc accgcaacag cctggacatc ttccggatcg |
| 9721 | ccaccccgca cagcctgggt agcgtgtgga agatccgagt gtggcacgac aacaaagggc |
| 9781 | tcagccctgc ctggttcctg cagcacgtca tcgtcaggga cctgcagacg gcacgcagcg |
| 9841 | ccttcttcct ggtcaatgac tggctttcgg tggagacgga ggccaacggg ggcctggtgg |
| 9901 | agaaggaggt gctggccgcg agcgacgcag cccttttgcg cttccggcgc ctgctggtgg |
| 9961 | ctgagctgca gcgtggcttc tttgacaagc acatctggct ctccatatgg gaccggccgc |
| 10021 | ctcgtagccg tttcactcgc atccagaggg ccacctgctg cgttctcctc atctgcctct |
| 10081 | tcctgggcgc caacgccgtg tggtacgggg ctgttggcga ctctgcctac agcacggggc |
| 10141 | atgtgtccag gctgagcccg ctgagcgtcg acacagtcgc tgttggcctg gtgtccagcg |
| 10201 | tggttgtcta tcccgtctac ctggccatcc tttttctctt ccggatgtcc cggagcaagg |
| 10261 | tggctgggag cccgagcccc acacctgccg ggcagcaggt gctggacatc gacagctgcc |
| 10321 | tggactcgtc cgtgctggac agctccttcc tcacgttctc aggcctccac gctgaggcct |
| 10381 | ttgttggaca gatgaagagt gacttgtttc tggatgattc taagagtctg gtgtgctggc |
| 10441 | cctccggcga gggaacgctc agttggccgg acctgctcag tgacccgtcc attgtgggta |
| 10501 | gcaatctgcg gcagctggca cggggccagg cgggccatgg gctgggccca gaggaggacg |
| 10561 | gcttctccct ggccagcccc tactcgcctg ccaaatcctt ctcagcatca gatgaagacc |
| 10621 | tgatccagca ggtccttgcc gagggggtca gcagcccagc ccctacccaa gacacccaca |
| 10681 | tggaaacgga cctgctcagc agcctgtcca gcactcctgg ggagaagaca gagacgctgg |
| 10741 | cgctgcagag gctgggggag ctggggccac ccagcccagg cctgaactgg gaacagcccc |
| 10801 | aggcagcgag gctgtccagg acaggactgg tggagggtct gcggaagcgc ctgctgccgg |
| 10861 | cctggtgtgc ctccctggcc cacgggctca gcctgctcct ggtggctgtg gctgtggctg |
| 10921 | tctcagggtg ggtgggtgcg agcttccccc cgggcgtgag tgttgcgtgg ctcctgtcca |
| 10981 | gcagcgccag cttcctggcc tcattcctcg gctgggagcc actgaaggtc ttgctggaag |
| 11041 | ccctgtactt ctcactggtg gccaagcggc tgcacccgga tgaagatgac accctggtag |
| 11101 | agagcccggc tgtgacgcct gtgagcgcac gtgtgccccg cgtacggcca ccccacggct |
| 11161 | ttgcactctt cctggccaag gaagaagccc gcaaggtcaa gaggctacat ggcatgctgc |
| 11221 | ggagcctcct ggtgtacatg ctttttctgc tggtgaccct gctggccagc tatggggatg |
| 11281 | cctcatgcca tgggcacgcc taccgtctgc aaagcgccat caagcaggag ctgcacagcc |
| 11341 | gggccttcct ggccatcacg cggtctgagg agctctggcc atggatggcc cacgtgctgc |
| 11401 | tgccctacgt ccacgggaac cagtccagcc cagagctggg gcccccacgg ctgcggcagg |
| 11461 | tgcggctgca ggaagcactc tacccagacc ctcccggccc cagggtccac acgtgctcgg |
| 11521 | ccgcaggagg cttcagcacc agcgattacg acgttggctg ggagagtcct cacaatggct |
| 11581 | cggggacgtg ggcctattca gcgccggatc tgctgggggc atggtcctgg ggctcctgtg |
| 11641 | ccgtgtatga cagcgggggc tacgtgcagg agctgggcct gagcctggag gagagccgcg |
| 11701 | accggctgcg cttcctgcag ctgcacaact ggctggacaa caggagccgc gctgtgttcc |
| 11761 | tggagctcac gcgctacagc ccggccgtgg ggctgcacgc cgccgtcacg ctgcgcctcg |
| 11821 | agttcccggc ggccggccgc gccctggccg ccctcagcgt ccgccccttt gcgctgcgcc |
| 11881 | gcctcagcgc gggcctctcg ctgcctctgc tcacctcggt gtgcctgctg ctgttcgccg |
| 11941 | tgcacttcgc cgtggccgag gcccgtactt ggcacaggga agggcgctgg cgcgtgctgc |
| 12001 | ggctcggagc ctgggcgcgg tggctgctgg tggcgctgac ggcggccacg gcactggtac |
| 12061 | gcctcgccca gctgggtgcc gctgaccgcc agtggacccg tttcgtgcgc ggccgcccgc |
| 12121 | gccgcttcac tagcttcgac caggtggcgc agctgagctc cgcagcccgt ggcctggcgg |
| 12181 | cctcgctgct cttcctgctt ttggtcaagg ctgcccagca gctacgcttc gtgcgccagt |
| 12241 | ggtccgtctt tggcaagaca ttatgccgag ctctgccaga gctcctgggg gtcaccttgg |
| 12301 | gcctggtggt gctcggggta gcctacgccc agctggccat cctgctcgtg tcttcctgtg |
| 12361 | tggactccct ctggagcgtg gcccaggccc tgttggtgct gtgccctggg actgggctct |
| 12421 | ctaccctgtg tcctgccgag tcctggcacc tgtcacccct gctgtgtgtg gggctctggg |
| 12481 | cactgcggct gtggggcgcc ctacggctgg gggctgttat tctccgctgg cgctaccacg |
| 12541 | ccttgcgtgg agagctgtac cggccggcct gggagcccca ggactacgag atggtggagt |
| 12601 | tgttcctgcg caggctgcgc ctctggatgg gcctcagcaa ggtcaaggag ttccgccaca |
| 12661 | aagtccgctt tgaagggatg gagccgctgc cctctcgctc ctccaggggc tccaaggtat |
| 12721 | ccccggatgt gcccccaccc agcgctggct ccgatgcctc gcacccctcc acctcctcca |
| 12781 | gccagctgga tgggctgagc gtgagcctgg gccggctggg gacaaggtgt gagcctgagc |
| 12841 | cctcccgcct ccaagccgtg ttcgaggccc tgctcaccca gtttgaccga ctcaaccagg |
| 12901 | ccacagagga cgtctaccag ctggagcagc agctgcacag cctgcaaggc cgcaggagca |
| 12961 | gccgggcgcc cgccggatct tcccgtggcc catccccggg cctgcggcca gcactgccca |
| 13021 | gccgccttgc ccgggccagt cggggtgtgg acctggccac tggccccagc aggacacccc |
| 13081 | ttcgggccaa gaacaaggtc caccccagca gcacttagtc ctccttcctg gcgggggtgg |
| 13141 | gccgtggagt cggagtggac accgctcagt attactttct gccgctgtca aggccgaggg |
| 13201 | ccaggcagaa tggctgcacg taggttcccc agagagcagg caggggcatc tgtctgtctg |
| 13261 | tgggcttcag cactttaaag aggctgtgtg gccaaccagg acccagggtc ccctccccag |
| 13321 | ctcccttggg aaggacacag cagtattgga cggtttctag cctctgagat gctaatttat |
| 13381 | ttccccgagt cctcaggtac agcgggctgt gcccggcccc accccctggg cagatgtccc |
| 13441 | ccactgctaa ggctgctggc ttcagggagg gttagcctgc accgccgcca ccctgcccct |
| 13501 | aagttattac ctctccagtt cctaccgtac tccctgcacc gtctcactgt gtgtctcgtg |
| 13561 | tcagtaattt atatggtgtt aaaatgtgta tatttttgta tgtcactatt ttcactaggg |
| 13621 | ctgaggggcc tgcgcccaga gctggcctcc cccaacacct gctgcgcttg gtaggtgtgg |
| 13681 | tggcgttatg gcagcccggc tgctgcttgg atgcgagctt ggccttgggc cggtgctggg |
| 13741 | ggcacagctg tctgccaggc actctcatca ccccagaggc cttgtcatcc tcccttgccc |
| 13801 | caggccaggt agcaagagag cagcgcccag gcctgctggc atcaggtctg ggcaagtagc |
| 13861 | aggactaggc atgtcagagg accccagggt ggttagagga aaagactcct cctgggggct |
| 13921 | ggctcccagg gtggaggaag gtgactgtgt gtgtgtgtgt gtgcgcgcgc gcacgcgcga |
| 13981 | gtgtgctgta tggcccaggc agcctcaagg ccctcggagc tggctgtgcc tgcttctgtg |
| 14041 | taccacttct gtgggcatgg ccgcttctag agcctcgaca cccccccaac ccccgcacca |
| 14101 | agcagacaaa gtcaataaaa gagctgtctg actgc |

ALAS1 (SEQ ID NO: 586) Homo sapiens 5′-aminolevulinate synthase 1
(ALAS1), transcript variant 1, mRNA.NM_000688

| | |
|---|---|
| 1 | cagaagaagg cagcgcccaa ggcgcatgcg cagcggtcac tcccgctgta tattaaggcg |
| 61 | ccggcgatcg cggcctgagg ctgctcccgg acaagggcaa cgagcgtttc gtttggactt |
| 121 | ctcgacttga gtgcccgcct ccttcgccgc cgcctctgca gtcctcagcg cagttatgcc |
| 181 | cagttcttcc cgctgtgggg acacgaccac ggaggaatcc ttgcttcagg gactcgggac |
| 241 | cctgctggac cccttcctcg ggtttagggg atgtggggac caggagaaag tcaggatccc |
| 301 | taagagtctt ccctgcctgg atggatgagt ggcttcttct ccacctagat tctttccaca |
| 361 | ggagccagca tacttcctga acatggagag tgttgttcgc cgctgcccat tcttatcccg |
| 421 | agtcccccag gcctttctgc agaaagcagg caaatctctg ttgttctatg cccaaaactg |
| 481 | ccccaagatg atggaagttg gggccaagcc agcccctcgg gcattgtcca ctgcagcagt |
| 541 | acactaccaa cagatcaaag aaacccctcc ggccagtgag aaagacaaaa ctgctaaggc |
| 601 | caaggtccaa cagactcctg atggatccca gcagagtcca gatggcacac agcttccgtc |
| 661 | tggacacccc ttgcctgcca caagccaggg cactgcaagc aaatgccctt tcctggcagc |
| 721 | acagatgaat cagagaggca gcagtgtctt ctgcaaagcc agtcttgagc ttcaggagga |
| 781 | tgtgcaggaa atgaatgccg tgaggaaaga ggttgctgaa acctcagcag gccccagtgt |
| 841 | ggttagtgtg aaaaccgatg gaggggatcc cagtggactg ctgaagaact tccaggacat |
| 901 | catgcaaaag caaagaccag aaagagtgtc tcatcttctt caagataact tgccaaaatc |
| 961 | tgtttccact tttcagtatg atcgtttctt tgagaaaaaa attgatgaga aaaagaatga |
| 1021 | ccacacctat cgagttttta aaactgtgaa ccggcgagca cacatcttcc ccatggcaga |
| 1081 | tgactattca gactccctca tcaccaaaaa gcaagtgtca gtctggtgca gtaatgacta |
| 1141 | cctaggaatg agtcgccacc cacgggtgtg tggggcagtt atggacactt tgaaacaaca |
| 1201 | tggtgctggg gcaggtggta ctagaaatat ttctggaact agtaaattcc atgtggactt |
| 1261 | agagcgggag ctggcagacc tccatgggaa agatgccgca ctcttgtttt cctcgtgctt |
| 1321 | tgtggccaat gactcaaccc tcttcaccct ggctaagatg atgccaggct gtgagattta |
| 1381 | ctctgattct gggaaccatg cctccatgat ccaagggatt cgaaacagcc gagtgccaaa |
| 1441 | gtacatcttc cgccacaatg atgtcagcca cctcagagaa ctgctgcaaa gatctgaccc |
| 1501 | ctcagtcccc aagattgtgg catttgaaac tgtccattca atggatgggg cggtgtgccc |
| 1561 | actggaagag ctgtgtgatg tggcccatga gtttggagca atcaccttcg tggatgaggt |
| 1621 | ccacgcagtg gggctttatg gggctcgagg cggagggatt ggggatcggg atggagtcat |
| 1681 | gccaaaaatg gacatcattt ctggaacact tggcaaagcc tttggttgtg ttggagggta |
| 1741 | catcgccagc acgagttctc tgattgacac cgtacggtcc tatgctgctg gcttcatctt |
| 1801 | caccacctct ctgccaccca tgctgctggc tggagccctg gagtctgtgc ggatcctgaa |
| 1861 | gagcgctgag ggacgggtgc ttcgccgcca gcaccagcgc aacgtcaaac tcatgagaca |
| 1921 | gatgctaatg gatgccggcc tccctgttgt ccactgcccc agccacatca tccctgtgcg |
| 1981 | ggttgcagat gctgctaaaa acacagaagt ctgtgatgaa ctaatgagca gacataacat |
| 2041 | ctacgtgcaa gcaatcaatt accctacggt gccccgggga gaagagctcc tacggattgc |
| 2101 | ccccacccct caccacacac cccagatgat gaactacttc cttgagaatc tgctagtcac |
| 2161 | atggaagcaa gtggggctgg aactgaagcc tcattcctca gctgagtgca acttctgcag |
| 2221 | gaggccactg cattttgaag tgatgagtga aagagagaag tcctatttct caggcttgag |
| 2281 | caagttggta tctgctcagg cctgagcatg acctcaatta tttcacttaa ccccaggcca |
| 2341 | ttatcatatc cagatggtct tcagagttgt ctttatatgt gaattaagtt atattaaatt |
| 2401 | ttaatctata gtaaaaacat agtcctggaa ataaattctt gcttaaatgg tgaaaaaa |

ALAS1 (SEQ ID NO: 587) Homo sapiens 5′-aminolevulinate synthase 1
(ALAS1), transcript Variant 2, mRNA. NM_199166

| | |
|---|---|
| 1 | cagaagaagg cagcgcccaa ggcgcatgcg cagcggtcac tcccgctgta tattaaggcg |
| 61 | ccggcgatcg cggcctgagg ctgctcccgg acaagggcaa cgagcgtttc gtttggactt |
| 121 | ctcgacttga gtgcccgcct ccttcgccgc cgcctctgca gtcctcagcg cagtctttcc |
| 181 | acaggagcca gcatacttcc tgaacatgga gagtgttgtt cgccgctgcc cattcttatc |
| 241 | ccgagtcccc caggcctttc tgcagaaagc aggcaaatct ctgttgttct atgcccaaaa |
| 301 | ctgccccaag atgatggaag ttggggccaa gccagcccct cgggcattgt ccactgcagc |
| 361 | agtacactac caacagatca aagaaacccc tccggccagt gagaaagaca aaactgctaa |
| 421 | ggccaaggtc caacagactc ctgatggatc ccagcagagt ccagatggca cacagcttcc |
| 481 | gtctggacac cccttgcctg ccacaagcca gggcactgca agcaaatgcc ctttcctggc |
| 541 | agcacagatg aatcagagag gcagcagtgt cttctgcaaa gccagtcttg agcttcagga |
| 601 | ggatgtgcag gaaatgaatg ccgtgaggaa agaggttgct gaaacctcag caggccccag |
| 661 | tgtggttagt gtgaaaaccg atggagggga tcccagtgga ctgctgaaga acttccagga |
| 721 | catcatgcaa aagcaaagac cagaaagagt gtctcatctt cttcaagata acttgccaaa |
| 781 | atctgtttcc acttttcagt atgatcgttt ctttgagaaa aaaattgatg agaaaaagaa |
| 841 | tgaccacacc tatcgagttt ttaaaactgt gaaccggcga gcacacatct tccccatggc |
| 901 | agatgactat tcagactccc tcatcaccaa aaagcaagtg tcagtctggt gcagtaatga |
| 961 | ctacctagga atgagtcgcc acccacgggt gtgtggggca gttatggaca ctttgaaaca |
| 1021 | acatggtgct ggggcaggtg gtactagaaa tatttctgga actagtaaat tccatgtgga |
| 1081 | cttagagcgg gagctggcag acctccatgg gaaagatgcc gcactcttgt tttcctcgtg |
| 1141 | ctttgtggcc aatgactcaa ccctcttcac cctggctaag atgatgccag gctgtgagat |
| 1201 | ttactctgat tctgggaacc atgcctccat gatccaaggg attcgaaaca gccgagtgcc |
| 1261 | aaagtacatc ttccgccaca atgatgtcag ccacctcaga gaactgctgc aaagatctga |
| 1321 | cccctcagtc cccaagattg tggcatttga aactgtccat tcaatggatg gggcggtgtg |
| 1381 | cccactggaa gagctgtgtg atgtggccca tgagtttgga gcaatcacct tcgtggatga |
| 1441 | ggtccacgca gtggggcttt atggggctcg aggcggaggg attggggatc gggatggagt |
| 1501 | catgccaaaa atggacatca tttctggaac acttggcaaa gcctttggtt gtgttggagg |
| 1561 | gtacatcgcc agcacgagtt ctctgattga caccgtacgg tcctatgctg ctggcttcat |
| 1621 | cttcaccacc tctctgccac ccatgctgct ggctggagcc ctggagtctg tgcggatcct |
| 1681 | gaagagcgct gagggacggg tgcttcgccg ccagcaccag cgcaacgtca aactcatgag |
| 1741 | acagatgcta atggatgccg gcctccctgt tgtccactgc cccagccaca tcatccctgt |
| 1801 | gcgggttgca gatgctgcta aaaacacaga agtctgtgat gaactaatga gcagacataa |
| 1861 | catctacgtg caagcaatca attaccctac ggtgccccgg ggagaagagc tcctacggat |
| 1921 | tgcccccacc cctcaccaca caccccagat gatgaactac ttccttgaga atctgctagt |
| 1981 | cacatggaag caagtggggc tggaactgaa gcctcattcc tcagctgagt gcaacttctg |
| 2041 | caggaggcca ctgcattttg aagtgatgag tgaaagagag aagtcctatt tctcaggctt |
| 2101 | gagcaagttg gtatctgctc aggcctgagc atgacctcaa ttatttcact taaccccagg |
| 2161 | ccattatcat atccagatgg tcttcagagt tgtctttata tgtgaattaa gttatattaa |
| 2221 | attttaatct atagtaaaaa catagtcctg gaaataaatt cttgcttaaa tggtgaaaaa |
| 2281 | a |

GTF2D1 (SEQ ID NO: 588) Homo sapiens TATA-box binding protein
(TBP), transcript variant 1, NM_003194

| | |
|---|---|
| 1 | ggcggaagtg acattatcaa cgcgcgccag gggttcagtg aggtcgggca ggttcgctgt |
| 61 | ggcgggcgcc tgggccgccg gctgtttaac ttcgcttccg ctggcccata gtgatctttg |
| 121 | cagtgaccca gcatcactgt ttcttggcgt gtgaagataa cccaaggaat tgaggaagtt |
| 181 | gctgagaaga gtgtgctgga gatgctctag gaaaaaattg aatagtgaga cgagttccag |
| 241 | cgcaagggtt tctggtttgc caagaagaaa gtgaacatca tggatcagaa caacagcctg |
| 301 | ccaccttacg ctcagggctt ggcctcccct cagggtgcca tgactcccgg aatccctatc |
| 361 | tttagtccaa tgatgcctta tggcactgga ctgaccccac agcctattca gaacaccaat |
| 421 | agtctgtcta ttttggaaga gcaacaaagg cagcagcagc aacaacaaca gcagcagcag |
| 481 | cagcagcagc agcaacagca acagcagcag cagcagcagc agcagcagca gcagcagcag |
| 541 | cagcagcagc agcagcagca acaggcagtg gcagctgcag ccgttcagca gtcaacgtcc |
| 601 | cagcaggcaa cacagggaac ctcaggccag gcaccacagc tcttccactc acagactctc |
| 661 | acaactgcac ccttgccggg caccactcca ctgtatccct cccccatgac tcccatgacc |
| 721 | cccatcactc ctgccacgcc agcttcggag agttctggga ttgtaccgca gctgcaaaat |
| 781 | attgtatcca cagtgaatct tggttgtaaa cttgacctaa agaccattgc acttcgtgcc |
| 841 | cgaaacgccg aatataatcc caagcggttt gctgcggtaa tcatgaggat aagagagcca |
| 901 | cgaaccacgg cactgatttt cagttctggg aaaatggtgt gcacaggagc caagagtgaa |
| 961 | gaacagtcca gactggcagc aagaaaatat gctagagttg tacagaagtt gggttttcca |
| 1021 | gctaagttct tggacttcaa gattcagaat atggtgggga gctgtgatgt gaagtttcct |
| 1081 | ataaggttag aaggccttgt gctcacccac caacaattta gtagttatga gccagagtta |
| 1141 | tttcctggtt taatctacag aatgatcaaa cccagaattg ttctccttat ttttgtttct |
| 1201 | ggaaaagttg tattaacagg tgctaaagtc agagcagaaa tttatgaagc atttgaaaac |
| 1261 | atctacccta ttctaaaggg attcaggaag acgacgtaat ggctctcatg tacccttgcc |
| 1321 | tcccccaccc ccttcttttt ttttttttaa acaaatcagt ttgttttggt acctttaaat |
| 1381 | ggtggtgttg tgagaagatg gatgttgagt tgcagggtgt ggcaccaggt gatgcccttc |
| 1441 | tgtaagtgcc caccgcggga tgccgggaag gggcattatt tgtgcactga gaacaccgcg |
| 1501 | cagcgtgact gtgagttgct cataccgtgc tgctatctgg gcagcgctgc ccatttattt |
| 1561 | atatgtagat tttaaacact gctgttgaca agttggtttg agggagaaaa ctttaagtgt |
| 1621 | taaagccacc tctataattg attggacttt ttaattttaa tgtttttccc catgaaccac |
| 1681 | agtttttata tttctaccag aaaagtaaaa atctttttta aaagtgttgt ttttctaatt |
| 1741 | tataactcct aggggttatt tctgtgccag acacattcca cctctccagt attgcaggac |
| 1801 | agaatatatg tgttaatgaa aatgaatggc tgtacatatt tttttctttc ttcagagtac |
| 1861 | tctgtacaat aaatgcagtt tataaaagtg ttagattgtt gttaaaaaaa aaaaaaaaaa |
| 1921 | a |

HMBS (SEQ ID NO: 589) Homo sapiens hydroxymethylbilane synthase
(HMBS), transcript variant 1. NM_000190

| | |
|---|---|
| 1 | ccggaagtga cgcgaggctc tgcggagacc aggagtcaga ctgtaggacg acctcgggtc |
| 61 | ccacgtgtcc ccggtactcg ccggccggag cccccggctt cccggggccg ggggacctta |
| 121 | gcggcaccca cacacagcct actttccaag cggagccatg tctggtaacg gcaatgcggc |
| 181 | tgcaacggcg gaagaaaaca gcccaaagat gagagtgatt cgcgtgggta cccgcaagag |
| 241 | ccagcttgct cgcatacaga cggacagtgt ggtggcaaca ttgaaagcct cgtaccctgg |
| 301 | cctgcagttt gaaatcattg ctatgtccac cacaggggac aagattcttg atactgcact |
| 361 | ctctaagatt ggagagaaaa gcctgtttac caaggagctt gaacatgccc tggagaagaa |
| 421 | tgaagtggac ctggttgttc actccttgaa ggacctgccc actgtgcttc ctcctggctt |
| 481 | caccatcgga gccatctgca agcgggaaaa ccctcatgat gctgttgtct ttcacccaaa |
| 541 | atttgttggg aagaccctag aaaccctgcc agagaagagt gtggtgggaa ccagctccct |
| 601 | gcgaagagca gcccagctgc agagaaagtt cccgcatctg gagttcagga gtattcgggg |
| 661 | aaacctcaac acccggcttc ggaagctgga cgagcagcag gagttcagtg ccatcatcct |
| 721 | ggcaacagct ggcctgcagc gcatgggctg gcacaaccgg gtggggcaga tcctgcaccc |
| 781 | tgaggaatgc atgtatgctg tgggccaggg ggccttgggc gtggaagtgc gagccaagga |
| 841 | ccaggacatc ttggatctgg tgggtgtgct gcacgatccc gagactctgc ttcgctgcat |
| 901 | cgctgaaagg gccttcctga ggcacctgga aggaggctgc agtgtgccag tagccgtgca |
| 961 | tacagctatg aaggatgggc aactgtacct gactggagga gtctggagtc tagacggctc |
| 1021 | agatagcata caagagacca tgcaggctac catccatgtc cctgcccagc atgaagatgg |
| 1081 | ccctgaggat gacccacagt tggtaggcat cactgctcgt aacattccac gagggcccca |
| 1141 | gttggctgcc cagaacttgg gcatcagcct ggccaacttg ttgctgagca aaggagccaa |
| 1201 | aaacatcctg gatgttgcac ggcagcttaa cgatgcccat taactggttt gtggggcaca |
| 1261 | gatgcctggg ttgctgctgt ccagtgccta catcccgggc ctcagtgccc cattctcact |
| 1321 | gctatctggg gagtgattac cccgggagac tgaactgcag ggttcaagcc ttccagggat |
| 1381 | ttgcctcacc ttggggcctt gatgactgcc ttgcctcctc agtatgtggg ggcttcatct |
| 1441 | ctttagagaa gtccaagcaa cagcctttga atgtaaccaa tcctactaat aaaccagttc |
| 1501 | tgaaggtgta aaaaaaaaaa aaaaaa |

1-13. (canceled)

14. A method for diagnosing whether a patient has a systemic inflammatory condition, comprising:
(i) determining the amount of FAM20A and OLAH in a sample obtained from a patient,
(ii) comparing the amount of FAM20A determined in said sample in (i) to a corresponding reference value representative of a healthy individual,
(iii) comparing the amount of OLAH determined in said sample in (i) to a corresponding reference value representative of a healthy individual;
wherein the patient is diagnosed as having a systemic inflammatory condition, when an increase is observed in FAM20A and/or OLAH in the sample obtained from the patient relative to the corresponding reference value; and
wherein the patient is diagnosed as not having a systemic inflammatory condition, when no increase is observed in FAM20A and OLAH, in the sample obtained from the patient relative to the corresponding reference value.

15-38. (canceled)

39. The method according to claim 14,
wherein the sample is a sample of blood, cerebral spinal fluid, cells, a cellular extract, a tissue specimen, or a tissue biopsy, or a combination thereof.

40. The method according to claim 39, wherein the blood sample is a sample of whole blood, purified peripheral blood leukocytes or cell type sorted leukocytes.

41. The method according to claim 14, wherein the method comprises determining the amount of FAM20A and OLAH at the protein level.

42. The method according to claim 41, wherein the amount of FAM20A is determined using an antibody specific for FAM20A, and the amount of OLAH is determined using an antibody specific for OLAH.

43. The method according to claim 14, wherein the method comprises determining the amount of FAM20A and OLAH at the nucleic acid level.

44. The method according to claim 43, wherein the amount of FAM20A is determined using an oligonucleotide specific for FAM20A, and the amount of OLAH is determined using an oligonucleotide specific for OLAH.

45-48. (canceled)

49. The method according to claim 44, wherein an oligonucleotide specific for FAM20A comprises or is complementary to a nucleic acid sequence having at least 80% sequence identity to the nucleic acid sequence of SEQ ID NO:94, 95, 96 or 97, preferably SEQ ID NO:94 or 95; or
wherein an oligonucleotide specific for FAM20A comprises or is complementary to a nucleic acid sequence having at least 80% sequence identity to the nucleic acid sequence of SEQ ID NO:424 or 427.

50. The method according to claim 43, wherein an oligonucleotide specific for OLAH comprises or is complementary to a nucleic acid sequence having at least 80% sequence identity to the nucleic acid sequence of SEQ ID NO:146, 147, 148, or 149, preferably SEQ ID NO:146; or
wherein an oligonucleotide specific for OLAH comprises or is complementary to a nucleic acid sequence having at least 80% sequence identity to the nucleic acid sequence of SEQ ID NO:430 or 433.

51. The method according to claim 44, wherein an oligonucleotide specific for OLAH comprises or is complementary to a nucleic acid sequence having at least 80% sequence identity to the nucleic acid sequence of SEQ ID NO:146, 147, 148, or 149, preferably SEQ ID NO:146; or
wherein an oligonucleotide specific for OLAH comprises or is complementary to a nucleic acid sequence having at least 80% sequence identity to the nucleic acid sequence of SEQ ID NO:430 or 433.
